(19) United States
(12) Patent Application Publication
(10) Pub. No.: US 2023/0265103 A1
(43) Pub. Date: Aug. 24, 2023

(54) NOVEL INHIBITORS OF PIKFYVE AND METHODS USING SAME

(71) Applicant: TME Therapeutics LLC, Acton, MA (US)

(72) Inventor: Vinod F. PATEL, Acton, MA (US)

(21) Appl. No.: 18/299,657

(22) Filed: Apr. 12, 2023

Related U.S. Application Data
(63) Continuation-in-part of application No. PCT/IB2022/059660, filed on Oct. 8, 2022.
(60) Provisional application No. 63/253,407, filed on Oct. 7, 2021.

(51) Int. Cl. C07D 495/04; C07D 513/04; C07D 473/32; C07D 471/04; C07D 519/00; C07D 413/14; C07D 498/04; A61P 31/14; A61P 31/16

(52) U.S. Cl. CPC C07D 495/04; C07D 513/04; C07D 473/32; C07D 471/04; C07D 519/00; C07D 413/14; C07D 498/04; A61P 31/14; A61P 31/16

(57) ABSTRACT

The invention relates to novel inhibitors of the PIKFYVE, a phosphoinositide kinase, useful for the treatment of diseases or disorders characterized by dysregulation of phosphoinositide-mediated signal transduction pathways, including hyperproliferative diseases (such as MET or RAS dependent cancers), autoimmune diseases, Crohn's disease, psoriasis, neurological diseases, diabetes, corneal fleck dystrophy, and viral infection (including HIV, Ebola, and coronavirus infections). The invention further relates to pharmaceutical compositions comprising PIKFYVE inhibitors and methods of treatment of such diseases and disorders.

### CROSS-REFERENCE TO RELATED APPLICATIONS

This application is continuation-in-part of international PCT application No. PCT/IB2022/059660, filed on Oct. 8, 2022, which claims priority to, and the benefit of, U.S. Provisional Application Ser. No. 63/253,407, filed on Oct. 7, 2021, the contents of which are hereby incorporated by reference in its entirety.

### FIELD OF THE INVENTION

The present disclosure relates to novel inhibitors of the PIKFYVE, a phosphoinositide kinase, useful for the treatment of diseases or disorders characterized by dysregulation of phosphoinositide-mediated signal transduction pathways, including hyperproliferative diseases (such as MET or RAS dependent cancers, including prostate cancer), autoimmune diseases, Crohn's disease, psoriasis, neurological diseases, diabetes, corneal fleck dystrophy, and viral infection (including HIV, Ebola, and coronavirus infections).

The invention further relates to pharmaceutical compositions comprising PIKFYVE inhibitors and methods of treatment of such diseases and disorders.

### BACKGROUND OF THE INVENTION

Protein kinases represent a large family of proteins which play a variety of crucial roles in the regulation of a wide range of cellular processes. Such kinases include lipid kinases, serine-threonine protein kinases, tyrosine protein kinases, and other kinases. Inhibition of various protein kinases, especially selective inhibition, has become an important strategy in treating many diseases and disorders.

PIKFYVE (or PIKfyve) is a phosphoinositide kinase whose primary function is the phosphorylation of phosphoinositide-3-phosphate (PtdIns3P or PI3P) to form phosphoinositide-3,5-diphosphate (PtdIns(3,5)P2 or PI(3,5P)2). PIKFYVE also phosphorylates phosphoinositide to form phosphoinositide-5-phosphate (PtdIns5P or PI5P). PIKFYVE includes an FYVE-finger domain, a zinc-finger domain, which is responsible for binding of the protein to PI3P. PI3P is a membrane bound lipid, and binding of PIKFYVE to PI3P can result in insertion of the kinase into cellular membranes, such as endosomes, vacuoles and other intracellular vesicles.

PI(3,5)P2 is one of seven phosphoinositides found in eukaryotic cell membranes, along with the more abundant PI3P, PI4P (phosphoinositide-4-phosphate), PI5P, PI(4,5)P2 (phosphoinositide-4,5-diphosphate), and PIP3 (phosphoinositide-3,4,5-triphosphate).

Phosphoinositides are membrane-bound regulatory lipids, and they participate in signaling events that control cytoskeletal dynamics, intracellular membrane trafficking, cell proliferation, and many other cellular functions. Like other phosphoinositides, PI(3,5)P2 acts as a signaling molecule in various cellular signaling pathways, as well as being a precursor for the synthesis of PI5P.

PI(3,5)P2 is present at the lowest concentration of the phosphoinositides and after formation is it is rapidly dephosphorylated back to PI3P by the phosphatase Sac3.

PIKFYVE is the only kinase which forms PI(3,5)P2 and unusually, PIKFYVE exists in a large multi-protein complex, the PAS complex, also comprising Sac3. The presence of a kinase and a phosphatase with opposite activities in the same complex suggests the critical importance of the concentration of PI(3,5)P2 to normal cell functioning. In addition to PIKFYVE and Sac1, the PAS complex also contains ArPIKFYVE, a regulatory protein which scaffolds the complex. Studies show that in most eukaryotic organisms, silencing or knockout of PIKFYVE function (or its equivalent) is lethal during embryonic development, further suggesting the critical importance of this protein and its product PI(3,5)P2.

PI(3,5)P2 helps regulate endosomal operations, such as membrane fission and fusion, that maintain endosomal homeostasis and support trafficking pathways throughout cells. Inhibition of PIKFYVE function in in-vitro cell studies shows the formation of numerous cytosolic vacuoles which grow larger over time, but such defects are shown to be reversible upon resupply of PI(3,5)P2 or functioning PIKFYVE. While homozygous knockout models of PIKFYVE are lethal, heterozygous knockout it not. This as well as other studies suggest that PIKFYVE activity, and consequently PI(3,5)P2 cellular concentration, is normally in excess of that required for normal cell functioning.

Under the sustained activation of glutamate receptors, PIKFYVE has also been shown to facilitate the lysosomal degradation of type 1.2 voltage-dependent calcium channels in neurons. This helps protect neurons from excitotoxicity, and suggests a role in treating or preventing central nervous system dysfunction. In neuroendocrine cells, PIKFYVE also negatively regulates calcium-dependent exocytosis. In addition, PIKFYVE has also been shown to phosphorylate Transcription Factor EB (TFEB), which may be related to the activity of PIKFYVE inhibitors in treating multiple myeloma.

PIKFYVE and PI(3,5)P2 have been linked to the pathogenesis of several diseases and disorders. PIKFYVE mutations are found in 8 out of 10 families with Francois-Neetens corneal fleck dystrophy. Interference with PIKFYVE function is associated with impaired glucose uptake. Studies in mice show that selective PIKFYVE disruption in skeletal muscle cells results in systemic insulin resistance, glucose intolerance, hyperinsulinemia and increased adiposity, all of which are signs of prediabetes in humans. This is further supported by studies showing that acute insulin treatment results in increases in PI(3,5)P2 concentration in adipocytes, and this promotes increased GLUT4 translocation and surface expression, increasing glucose transport into cells. In various other cell and animal models, PI(3,5)P2 has been shown to be elevated by hyperosmotic shock in adipocytes, mitogenic signals (such as IL-2 and UV light in lymphocytes), protein kinase C activation in platelets, and epidermal growth factor stimulation of COS cells.

Several small molecule inhibitors of PIKFYVE have recently been reported, including Apilimod. Apilimod, studied as an autoimmune disease treatment (Crohn's disease, rheumatoid arthritis) was originally identified as an inhibitor of IL-12 and IL-23 synthesis, but was later found to also have potent PIKFYVE inhibitory activity. It is suspected that Apilimod's activity may have been due to PIKFYVE inhibition rather than interreference with IL-12 and IL-23. PIKFYVE inhibitors have also shown promise as cancer therapies, in particular, for the treatment of non-Hodgkin lymphoma, multiple myeloma, melanoma, liver cancer, and glioblastoma. PIKFYVE inhibition has also shown promise as a therapy for amyotrophic lateral sclerosis (ALS) and fronto-temporal dementia (FTD), in particular ALS and FTD marked by repeat expansions of the C9ORF72 gene (C9FTD/ALS). PIKFYVE inhibition has also been suggested to be useful in the downstream inhibition of RANK signaling (receptor activator of nuclear factor kappa N), which plays an important role in bone remodeling and may be useful in treating bone resorption in multiple myeloma, prostate cancer and breast cancer patients.

Because of the critical role of intracellular vesicle trafficking in the life cycle of some eukaryotic viruses, PIKFYVE inhibitors also have been found effective in inhibiting viral infection. Enveloped viruses have a life cycle that begins with binding of a viral surface protein to a specific extracellular membrane protein on the target cell. For some viruses, such as HIV, receptor binding triggers fusion of the viral envelope with the cell membrane, resulting in deposition of the viral nucleoprotein complex into the cytoplasm. However, for other viruses, including Ebola, influenza A, vesicular stomatitis virus, Lassa fever virus, lymphocytic choriomeningitis virus, and coronaviruses (including MERS-CoV, SARS-CoV and SARS-CoV-2), receptor binding triggers endocytosis of the entire viral particle. The resulting endosome is transported within the cell until something triggers fusion of the viral envelope with the endosome membrane, resulting in deposition of the viral nucleoprotein complex into the cytoplasm. The triggering event can be acidification of the endosome or proteolysis of viral surface proteins.

Apilimod and other PIKFYVE inhibitors have been found to prevent infection by some of these enveloped viruses, either by interfering with endosome formation or by blocking endosome trafficking or otherwise preventing the triggering of endosome-viral envelope fusion.

There continues to be a need for new, selective inhibitors of PIKFYVE. The present disclosure provides novel, highly effective small-molecule inhibitors of PIKFYVE.

### SUMMARY OF THE INVENTION

Therefore, first aspect, the invention provides a compound of Formula I:

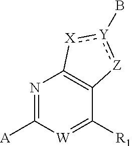

in free or pharmaceutically acceptable salt form, wherein
(i) X is selected from —CH—, —CR₃—, —S—, —O—, —N—, —NH—, and —NR₃—;
(ii) Y is selected from —C— and —N—;
(iii) Z is selected from —CH—, —CR₃—, —S—, —O—, —N—, —NH—, and —NR₃—;
(iv) W is —CH—, —CR₄—, or —N—;
(v) A is an optionally substituted heteroaryl (e.g., 5-membered heteroaryl) or heterocycloalkyl (e.g., 3- to 6-membered heterocycloalkyl);
(vi) B is halo, an optionally substituted aryl, optionally substituted heteroaryl, optionally substituted C₃₋₆cycloalkyl, optionally substituted 3- to 6-membered heterocycloalkyl, optionally substituted 3- to 6-membered heterocycloalkenyl, optionally substituted C₁₋₆alkyl, optionally substituted C₂₋₆alkenyl (e.g., vinyl), —N(Rₐ)—R₂, —O—R₂, —(CO)—R₂, —(CO)—O—R₂, —(CO)—N(Rₐ)—R₂, —O—(CO)—R₂, —N(Rₐ)—(CO)—R₂, —(CO)—N(Rₐ)—(CO)—R₂, N(Rₐ)—(CO)—N(Rₐ)—R₂, optionally substituted —(C₁₋₆alkyl)-(3- to 6-membered heterocycloalkyl), optionally substituted —(C₁₋₆alkyl)-(C₃₋₆cycloalkyl), optionally substituted —(C₂₋₆alkenyl)-(3- to 6-membered heterocycloalkyl), optionally substituted —(C₂₋₆alkenyl)-(C₃₋₆cycloalkyl), optionally substituted —(C₂₋₆alkynyl)-(3- to 6-membered heterocycloalkyl), optionally substituted —(C₂₋₆alkynyl)-(C₃₋₆cycloalkyl), optionally substituted —(C₁₋₆alkyl)-N(Rₐ)—R₂, optionally substituted —(C₁₋₆alkyl)-O—R₂, optionally substituted —(C₂₋₆alkenyl)-N(Rₐ)—R₂, optionally substituted —(C₂₋₆alkenyl)-O—R₂, optionally substituted —(C₂₋₆alkenyl)-N(Rₐ)(CO)—R₂, optionally substituted —(C₂₋₆alkenyl)-O(CO)—R₂, optionally substituted —(C₂₋₆alkynyl)-N(Rₐ)—R₂, optionally substituted —(C₂₋₆alkynyl)-O—R₂, optionally substituted —(C₂₋₆alkynyl)-N(Rₐ)(CO)—R₂, optionally substituted —(C₂₋₆alkynyl)-O(CO)—R₂, optionally substituted —(C₁₋₆alkyl)-(CO)—N(Rₐ)—R₂, optionally substituted —O—(C₁₋₆alkyl)-R₂, optionally substituted —N(Rₐ)—(C₁₋₆alkyl)-R₂, optionally substituted —CH₂-(3- to 6-membered heterocycloalkyl), optionally substituted —CH₂—(C₃₋₆cycloalkyl), optionally substituted —CH₂—N(Rₐ)—R₂, optionally substituted —CH₂—O—R₂, or optionally substituted —CH₂—(CO)—N(Rₐ)—R₂; optionally substituted —(CO)-(3- to 6-membered heterocycloalkyl), optionally substituted —(CO)—(C₃₋₆cycloalkyl), optionally substituted (6- to 12-membered bicyclic heterocycloalkyl), optionally substituted —CH₂—(6- to 12-membered bicyclic heterocycloalkyl), or optionally substituted —(CO)-(6- to 12-membered bicyclic heterocycloalkyl);
(vii) R₁ is an optionally substituted C₁₋₆alkyl, optionally substituted C₃₋₆cycloalkyl, optionally substituted C₁₋₆alkoxy, optionally substituted 3- to 7-membered heterocycloalkyl, optionally substituted 6- to 12-membered bicyclic heterocycloalkyl), —C(O)—R₂, —C(O)O—R₂, —OC(O)—R₂, —C(O)N(Rₐ)—R₂, —N(Rₐ)C(O)—R₂, —N(Rₐ)—R₂, or —O—R₂;
(viii) Rₐ is H, optionally substituted C₁₋₆alkyl, or optionally substituted C₃₋₆cycloalkyl; and
(ix) R₂ is optionally substituted C₁₋₆alkyl, optionally substituted C₃₋₆cycloalkyl, optionally substituted C₁₋₆alkoxy, optionally substituted aryl, optionally substituted heteroaryl, or optionally substituted 3- to 7-membered heterocycloalkyl;
(x) R₃ is H, optionally substituted C₁₋₆alkyl (e.g., methyl), optionally substituted C₃₋₆ cycloalkyl, optionally substituted haloC₁₋₆alkyl (e.g., CF₃), or optionally substituted 3- to 6-membered heterocycloalkyl; and
(xi) R₄ is halogen (e.g., fluoro), —OH, —NH₂, C₁₋₆alkyl (e.g., methyl), C₃₋₆cycloalkyl (e.g., isopropyl), haloC₁₋₆alkyl (e.g., CF₃), C₁₋₆alkoxy (e.g., methoxy), —NH(C₁₋₆alkyl) (e.g., methylamino), or —N(C₁₋₆alkyl)(C₁₋₆alkyl) (e.g., dimethylamino); provided that
   (a) when A is an optionally substituted pyrazole, said pyrazole is substituted by at least one optionally substituted aryl (e.g., phenyl) ring;
   (b) when A is optionally substituted pyrazol-1-yl, X is not O when Y is —CH— and Z is —CH—;
   (c) when A is optionally substituted pyrazol-1-yl, and X is —CH—, Y is —C—, and Z is —O—, B is not pyridyl, pyrimidinyl, any 5-membered heteroaromatic ring (e.g., thiazolyl, oxazolyl, pyrazolyl, isoxazolyl, isothiazolyl or imidazolyl), or —(CO)—N(Rₐ)—R₂;
   (d) when A is 3-(m-tolyl)-pyrazol-1-yl, and X is —CH—, Y is —C—, and Z is —O—, and B is —(CO)-(6-12 membered heterocycloalkyl), said heterocycloalkyl in group B is not unsubstituted morpholine, unsubstituted piperidine, unsubstituted pyrrolidine, unsubstituted piperazine, or 4-methylpiperazine;
   (e) when B is W is —N—, X is —N—, —NH—, or —NCH₃—, or Z is —N—, —NH—, or —NCH₃—, and Y is —C—, then B is not pyridyl, unsubstituted pyrrolidinyl, unsubstituted piperidinyl, 1-methyl-4-piperidinyl, 2-methyl-4-piperidinyl, tetrahydropyranyl, dihydropyranyl, 1-tert-butoxycarbonyl-4-azetidinyl, 1-methyl-4-azetidinyl, 4-azetidinyl, 1,2-dihydroxy-1-ethyl;
   (f) when W is —CH—, X is —CH— and Y is —N—, B is not 2-(1-methyl-pyrazol-3-yl)ethyl;
   (g) when B is pyrid-4-yl, R₁ is morpholin-4-yl, X is —CH—, Y is —C—, and Z is —S—, A is not 3-(3-methylphenyl)-5-hydroxy-pyrazol-1-yl, 3-(3-methoxyphenyl)-5-hydroxy-pyrazol-1-yl, or 3-(3-isopropoxyphenyl)-5-hydroxy-pyrazol-1-yl; and
   (h) when B is phenyl, R₁ is N-cyclohexylamino, X is —O—, Y is —C—, and Z is —CH—, A is not 3,5-diphenyl-pyrazol-1-yl, 3-methyl-5-phenyl-pyrazol-1-yl, 3-trifluoromethyl-5-phenyl-pyrazol-1-yl, or 3,5-dimethyl-4-phenyl-pyrazol-1-yl;
   (i) when W is —N—, X is —CH— or —C(Me)-, Y is —C—, Z is —S— or —O—, and R₁ is 4-morpholinyl, then A is not 1H-indazol-4-yl, 1H-indol-4-yl, 1H-pyrrolo[2,3-b]pyridin-5-yl, or 2-methyl-3H-imidazo[4,5]pyridin-6-yl;
   (j) when W is —N—, X is —S— or —O—, Y is —C—, Z is —CH— or —C(Me)-, and R₁ is 4-morpholinyl, then A is not 1H-indazol-4-yl, 1H-indol-4-yl, 1H-pyrrolo[2,3-b]pyridin-5-yl, or 2-methyl-3H-imidazo[4,5]pyridin-6-yl;
   (k) when W is —N—, X is —CH— or —C(Me)-, Y is —C—, Z is —S— or —O—, B is H or optionally substituted C₁₋₆alkyl, and R₁ is 4-morpholinyl, then A is not 2-oxoindolin-4-yl, 1-acetylindolin-4-yl, 1H-indazol-6-yl, 1H-indol-5-yl, 1H-indol-6-yl, or quinolin-3-yl;
   (l) when W is —N—, X is —S— or —O—, Y is —C—, Z is —CH— or —C(Me)-, B is H or optionally substituted C₁₋₆alkyl, and R₁ is 4-morpholinyl, then A is not 2-oxoindolin-4-yl, 1-acetylindolin-4-yl, 1H-indazol-6-yl, 1H-indol-5-yl, 1H-indol-6-yl, or quinolin-3-yl;
   (m) when W is —N—, X is —S—, Z is —N—, Y is —C—, and R₁ is 4-morpholinyl, then A is not optionally substituted pyrid-3-yl, optionally substituted pyrimidin-5-yl, unsubstituted quinolin-3-yl, unsubstituted 1H-indazol-4-yl, unsubstituted 1H-indol-4-yl, or unsubstituted 1H-pyrrolo[2,3-b]pyridin-5-yl;
   (n) when W is —N—, X is —N—, Z is —S—, Y is —C—, and R₁ is 4-morpholinyl, then A is not optionally substituted pyrid-3-yl, optionally substituted pyrimidin-5-yl, unsubstituted quinolin-3-yl, unsubstituted 1H-indazol-4-yl, unsubstituted 1H-indol-4-yl, or unsubstituted 1H-pyrrolo[2,3-b]pyridin-5-yl,
   (o) when W is —N—, X is —S—, Z is —N—, Y is —C—, and A is unsubstituted indolyl, pyrazolyl, imidazolyl, or triazolyl, then R₁ is not 3-methylmorpholin-4-yl
   (p) when W is —N—, X is —NR₃—, R₃ is selected from methyl, ethyl, isopropyl, and cyclopentyl, Z is —N—, Y is —C—, and R₁ is 4-morpholinyl, then A is not benzo[d][1,3]dioxol-5-yl, pyrimidin-5-yl, 2-aminopyrimidin-5-yl, pyridin-3-yl, 6-aminopyridin-3-yl, 6-methoxypyridin-3-yl, quinolin-3-yl, or pyridin-4-yl;
   (q) when W is —N—, X is —NR₃—, R₃ is 1-benzylpiperidin-4-yl, Z is —CH—, Y is —N—, and R₁ is 4-morpholinyl, then A is not pyridin-3-yl, pyridin-4-yl, quinolin-3-yl, quinolin-6-yl, 1H-indol-2-yl, 1H-indol-5-yl, 1H-indol-6-yl, 1H-indol-7-yl, 1-methyl-indol-5-yl, or 2-oxo-2,3-dihydro-1H-benzo[d]imidazol-5-yl;
   (r) when W is —N—, X is —CH— or —C(Me)-, Y is —C—, Z is —S— or —O—, and R₁ is 4-morpholinyl, then A is not 2-aminopyrimidin-5-yl;
   (s) when W is —N—, Y is —C—, and R₁ is 4-morpholinyl, A is not 2-methyl-1H-benzo[d]imidazole-1-yl or 2-ethyl-1H-benzo[d]imidazol-1-yl;
   (t) when W is —N—, X is —NR₃—, R₃ is methyl, Z is —N—, Y is —C—, and B is optionally substituted —CH₂-piperidine, optionally substituted —CH₂-piperazine, —CH₂-azetidine, or 2,5-diazabicyclo[2.2.1]heptan-2-yl, then A is not 2-methyl-1H-benzo[d]imidazol-1-yl or 2-ethyl-1H-benzo[d]imidazol-1-yl;
   (u) when W is —N—, X is —CH—, Y is —C—, Z is —S—, and R₁ is 4-morpholinyl, then A is not 5-fluoro-1H-indol-4-yl, 6-fluoro-1H-indol-4-yl, 6-cyano-1H-indol-4-yl, 5,7-difluoro-1H-indol-4-yl, 2-methyl-1H-indol-4-yl, 1-methyl-1H-indol-4-yl, 1H-pyrrolo[2,3-c]pyridin-4-yl, 1H-pyrrolo[2,3-b]pyridin-4-yl, 2-oxo-1H-indolin-4-yl, imidazo[1,2-a]pyridin-5-yl, 2-methyl-quinolin-5-yl, 2-methylbenzo[b]thiophen-3-yl, 1H-indazol-3-yl, 1H-indol-3-yl, 1H-benzo[d]imidazole-1-yl, 1H-indazol-1-yl, 1H-indol-1-yl, 2-methyl-benzo[d]imidazole-1-yl, or 3-methylbenzo[d]imidazole-1-yl;
   (v) when W is —N—, X is —CH— or —C(Me)-, Y is —C—, Z is —S— or —O—, and R₁ is 4-morpholinyl, then A is not optionally substituted 1H-benzo[d]imidazol-1-yl, optionally substituted quinolin-5-yl, optionally substituted 1H-pyrrolo[2,3-c]pyridin-4-yl, optionally substituted 1H-pyrrolo[2,3-c]pyridin-3-yl, optionally substituted 1H-pyrrolo[3,2-c]pyridin-4-yl, optionally substituted isoquinolin-8-yl, optionally substituted isoquinolin-4-yl, optionally substituted isoquinolin-5-yl, optionally substituted 1H-indazol-1-yl, optionally substituted 1H-indazol-3-yl, optionally substituted benzofuran-3-yl, optionally substituted 1H-pyrrolo[2,3-b]pyridin-3-yl, optionally substituted 1H-pyrrolo[2,3-b]pyridin-5-yl, optionally substituted 1H-pyrrolo[2,3-b]pyridin-4-yl, optionally substituted benzo[b]thiophen-3-yl, optionally substituted 1H-pyrolo[3,2-c]-pyridin-1-yl, 2-oxo-1H-benzo[d]imidazol-1-yl, optionally substituted 1H-benzo[d]imidazol-1-yl, optionally substituted 2H-indazol-3-yl, 2-oxo-1-indolin-1-yl, optionally substituted 2H-indazol-3-yl, optionally substituted imidazo[3,2-d]pyrimidin-6-yl, optionally substituted imidazo[1,5-a]pyridin-8-yl, optionally substituted imidazo[1,2-a]pyridin-5-yl, optionally substituted imidazo[4,5-c]pyridin-1-yl, optionally substituted imidazo[4,5-c]pyridin-3-yl, 1-oxo-1,2-dihydroisoquinolin-4-yl, 2-methyl-3-oxo-2,3-dihydro-1H-indazol-1-yl, [1,2,4]-triazolo[4,3-a]pyridin-5-yl, cinnolin-4-yl, or benzo[d]isothiazol-3-yl;
   (w) when W is —N—, X is —S—, —NH— or —N(Me)-, Y is —C—, Z is —N—, and R₁ is 4-morpholinyl, then A is not optionally substituted 1H-benzo[d]imidazol-1-yl, optionally substituted quinolin-5-yl, optionally substituted 1H-pyrrolo[2,3-c]pyridin-4-yl, optionally substituted 1H-pyrrolo[2,3-c]pyridin-3-yl, optionally substituted 1H-pyrrolo[3,2-c]pyridin-4-yl, optionally substituted isoquinolin-8-yl, optionally substituted isoquinolin-4-yl, optionally substituted isoquinolin-5-yl, optionally substituted 1H-indazol-1-yl, optionally substituted 1H-indazol-3-yl, optionally substituted benzofuran-3-yl, optionally substituted 1H-pyrrolo[2,3-b]pyridin-3-yl, optionally substituted 1H-pyrrolo[2,3-b]pyridin-5-yl, optionally substituted 1H-pyrrolo[2,3-b]pyridin-4-yl, optionally substituted benzo[b]thiophen-3-yl, optionally substituted 1H-pyrolo[3,2-c]-pyridin-1-yl, 2-oxo-1H-benzo[d]imidazol-1-yl, optionally substituted 1H-benzo[d]imidazol-1-yl, optionally substituted 2H-indazol-3-yl, 2-oxo-1-indolin-1-yl, optionally substituted 2H-indazol-3-yl, optionally substituted imidazo[3,2-d]pyrimidin-6-yl, optionally substituted imidazo[1,5-a]pyridin-8-yl, optionally substituted imidazo[1,2-a]pyridin-5-yl, optionally substituted imidazo[4,5-c]pyridin-1-yl, optionally substituted imidazo[4,5-c]pyridin-3-yl, 1-oxo-1,2-dihydroisoquinolin-4-yl, 2-methyl-3-oxo-2,3-dihydro-1H-indazol-1-yl, [1,2,4]-triazolo[4,3-a]pyridin-5-yl, cinnolin-4-yl, or benzo[d]isothiazol-3-yl;
   (x) when W is —N—, X is —NH—, Y is —C—, Z is —S—, and R₁ is 4-morpholinyl, then A is not optionally substituted 1H-benzo[d]imidazol-1-yl, optionally substituted quinolin-5-yl, optionally substituted 1H-pyrrolo[2,3-c]pyridin-4-yl, optionally substituted 1H-pyrrolo[2,3-c]pyridin-3-yl, optionally substituted 1H-pyrrolo[3,2-c]pyridin-4-yl, optionally substituted isoquinolin-8-yl, optionally substituted isoquinolin-4-yl, optionally substituted isoquinolin-5-yl, optionally substituted 1H-indazol-1-yl, optionally substituted 1H-indazol-3-yl, optionally substituted benzofuran-3-yl, optionally substituted 1H-pyrrolo[2,3-b]pyridin-3-yl, optionally substituted 1H-pyrrolo[2,3-b]pyridin-5-yl, optionally substituted 1H-pyrrolo[2,3-b]pyridin-4-yl, optionally substituted benzo[b]thiophen-3-yl, optionally substituted 1H-pyrolo[3,2-c]-pyridin-1-yl, 2-oxo-1H-benzo[d]imidazol-1-yl, optionally substituted 1H-benzo[d]imidazol-1-yl, optionally substituted 2H-indazol-3-yl, 2-oxo-1-indolin-1-yl, optionally substituted 2H-indazol-3-yl, optionally substituted imidazo[3,2-d]pyrimidin-6-yl, optionally substituted imidazo[1,5-a]pyridin-8-yl, optionally substituted imidazo[1,2-a]pyridin-5-yl, optionally substituted imidazo[4,5-c]pyridin-1-yl, optionally substituted imidazo[4,5-c]pyridin-3-yl, 1-oxo-1,2-dihydroisoquinolin-4-yl, 2-methyl-3-oxo-2,3-dihydro-1H-indazol-1-yl, [1,2,4]-triazolo[4,3-a]pyridin-5-yl, cinnolin-4-yl, or benzo[d]isothiazol-3-yl;
   (y) when W is —N—, X is —N—, —NH— or —N(CH₂CH₂OH)—, Z is —N—, Y is —C—, and R₁ is 4-morpholinyl, then A is not 1H-indol-4-yl or 5-fluoro-1H-indol-4-yl;
   (z) when W is —N— or —CH—, X is —NH—, —N(Me)-, —N(Et)-, or —N(CH₂-cyclopropyl)-, Y is —C—, Z is —N—, and R₁ is 4-morpholinyl, then A is not an unsubstituted pyrazol-4-yl or a pyrazol-4-yl substituted by one or more groups selected from methyl, ethyl, n-propyl, isopropyl, and trifluoromethyl;
   (aa) when W is —N—, X is —NH—, Y is —C—, Z is —CH— or —N—, and R₁ is 4-morpholinyl, 3-methylmorpholin-4-yl, 3,3-dimethylmorpholin-4-yl, 2-oxa-5-azabicyclo[2.2.1]hept-5-yl, or 3-oxa-8-azabicyclo[3.2.1]oct-8-yl, then A is not 4-morpholinyl, 3-methylmorpholin-4-yl, piperidin-1-yl, 4-hydroxypiperidin-1-yl, 4-methoxypiperidin-1-yl, 4,4-difluoropiperidin-1-yl, or 8-oxa-3-azabicyclo[3.2.1]oct-3-yl;
   (bb) when W is —CH—, X is —S—, Y is —C—, and X is —CH—, B is not pyrazol-5-yl when A is pyrrolidine-1-yl;
      and wherein the compound of Formula I is not
4-(2-methyl-5-(3-(m-tolyl)-1H-pyrazol-1-yl)-1H-pyrrolo[3,2-b]pyridin-7-yl)morpholine,
2-(2-(2-aminopyrimidin-5-yl)-9-(2-hydroxyethyl)-6-morpholino-9H-purin-8-yl)propan-2-ol,
1-ethyl-3-(5-(6-(3-ethylmorpholino)-7-methyl-7H-purin-2-yl)pyrimidin-2-yl)urea, or
5-pyrrolidino-2-(4-methoxyphenyl)-2H-[1,2,3]triazolo[4,5-d]pyrimidin-7-one or a tautomer thereof.

In a second aspect, the invention provides a pharmaceutical composition comprising the compound of Formula I, in free or pharmaceutically acceptable salt form, in admixture with a pharmaceutically acceptable diluent or carrier.

In a third aspect, the invention provides a method for the treatment or prophylaxis of a disease or disorder characterized by dysregulation of phosphoinositide-mediated signal transduction pathways or which may be ameliorated by modulating (e.g., inhibiting) PIKFYVE-dependent signaling pathways or by modulating (e.g., inhibiting) endosome formation or trafficking, comprising administering to a patient in need thereof an effective amount of the compound of Formula I, in free or pharmaceutically acceptable salt form.

### DETAILED DESCRIPTION

In a first aspect, the invention provides a compound of Formula I:

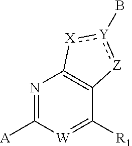

in free or pharmaceutically acceptable salt form, wherein
(i) X is selected from —CH—, —CR₃—, —S—, —O—, —N—, —NH—, and —NR₃—;
(ii) Y is selected from —C— and —N—;
(iii) Z is selected from —CH—, —CR₃—, —S—, —O—, —N—, —NH—, and —NR₃—;
(iv) W is —CH—, —CR₄—, or —N—;
(v) A is an optionally substituted heteroaryl (e.g., 5-membered heteroaryl) or heterocycloalkyl (e.g., 3- to 6-membered heterocycloalkyl);
(vi) B is halo, an optionally substituted aryl, optionally substituted heteroaryl, optionally substituted C₃₋₆cycloalkyl, optionally substituted 3- to 6-membered heterocycloalkyl, optionally substituted 3- to 6-membered heterocycloalkenyl, optionally substituted C₁₋₆alkyl, optionally substituted C₂₋₆alkenyl (e.g., vinyl), —N(Rₐ)—R₂, —O—R₂, —(CO)—R₂, —(CO)—O—R₂, —(CO)—N(Rₐ)—R₂, —O—(CO)—R₂, —N(Rₐ)—(CO)—R₂, —(CO)—N(Rₐ)—(CO)—R₂, N(Rₐ)—(CO)—N(Rₐ)—R₂, optionally substituted —(C₁₋₆alkyl)-(3- to 6-membered heterocycloalkyl), optionally substituted —(C₁₋₆alkyl)-(C₃₋₆cycloalkyl), optionally substituted —(C₂₋₆alkenyl)-(3- to 6-membered heterocycloalkyl), optionally substituted —(C₂₋₆alkenyl)-(C₃₋₆cycloalkyl), optionally substituted —(C₂₋₆alkynyl)-(3- to 6-membered heterocycloalkyl), optionally substituted —(C₂₋₆alkynyl)-(C₃₋₆cycloalkyl), optionally substituted —(C₁₋₆alkyl)-N(Rₐ)—R₂, optionally substituted —(C₁₋₆alkyl)-O—R₂, optionally substituted —(C₂₋₆alkenyl)-N(Rₐ)—R₂, optionally substituted —(C₂₋₆alkenyl)-O—R₂, optionally substituted —(C₂₋₆alkenyl)-N(Rₐ)(CO)—R₂, optionally substituted —(C₂₋₆alkenyl)-O(CO)—R₂, optionally substituted —(C₂₋₆alkynyl)-N(Rₐ)—R₂, optionally substituted —(C₂₋₆alkynyl)-O—R₂, optionally substituted —(C₂₋₆alkynyl)-N(Rₐ)(CO)—R₂, optionally substituted —(C₂₋₆alkynyl)-O(CO)—R₂, optionally substituted —(C₁₋₆alkyl)-(CO)—N(Rₐ)—R₂, optionally substituted —O—(C₁₋₆alkyl)-R₂, optionally substituted —N(Rₐ)—(C₁₋₆alkyl)-R₂, optionally substituted —CH₂—(3- to 6-membered heterocycloalkyl), optionally substituted —CH₂—(C₃₋₆cycloalkyl), optionally substituted —CH₂—N(Rₐ)—R₂, optionally substituted —CH₂—O—R₂, optionally substituted —CH₂—(CO)—N(Rₐ)—R₂, optionally substituted —(CO)-(3- to 6-membered heterocycloalkyl), optionally substituted —(CO)—(C₃₋₆cycloalkyl), optionally substituted (6- to 12-membered bicyclic heterocycloalkyl), optionally substituted —CH₂—(6- to 12-membered bicyclic heterocycloalkyl), or optionally substituted —(CO)-(6- to 12-membered bicyclic heterocycloalkyl);
(vii) R₁ is an optionally substituted C₁₋₆alkyl, optionally substituted C₃₋₆cycloalkyl, optionally substituted C₁₋₆alkoxy, optionally substituted 3- to 7-membered heterocycloalkyl, optionally substituted 6- to 12-membered bicyclic heterocycloalkyl), —C(O)—R₂, —C(O)O—R₂, —OC(O)—R₂, —C(O)N(Rₐ)—R₂, —N(Rₐ)C(O)—R₂, —N(Rₐ)—R₂, or —O—R₂;
(viii) Rₐ is H, optionally substituted C₁₋₆alkyl, or optionally substituted C₃₋₆cycloalkyl; and
(ix) R₂ is optionally substituted C₁₋₆alkyl, optionally substituted C₃₋₆cycloalkyl, optionally substituted C₁₋₆alkoxy, optionally substituted aryl, optionally substituted heteroaryl, or optionally substituted 3- to 7-membered heterocycloalkyl;
(x) R₃ is H, optionally substituted C₁₋₆alkyl (e.g., methyl), optionally substituted C₃₋₆cycloalkyl, optionally substituted haloC₁₋₆alkyl (e.g., CF₃), or optionally substituted 3- to 6-membered heterocycloalkyl; and
(xi) R₄ is halogen (e.g., fluoro), —OH, —NH₂, C₁₋₆alkyl (e.g., methyl), C₃₋₆cycloalkyl (e.g., isopropyl), haloC₁₋₆alkyl (e.g., CF₃), C₁₋₆alkoxy (e.g., methoxy), —NH(C₁₋₆alkyl) (e.g., methylamino), or —N(C₁₋₆alkyl)(C₁₋₆alkyl) (e.g., dimethylamino); provided that
   (a) when A is an optionally substituted pyrazole, said pyrazole is substituted by at least one optionally substituted aryl (e.g., phenyl) ring;
   (b) when A is optionally substituted pyrazol-1-yl, X is not O when Y is —CH— and Z is —CH—;
   (c) when A is optionally substituted pyrazol-1-yl, and X is —CH—, Y is —C—, and Z is —O—, B is not pyridyl, pyrimidinyl, any 5-membered heteroaromatic ring (e.g., thiazolyl, oxazolyl, pyrazolyl, isoxazolyl, isothiazolyl or imidazolyl), or —(CO)—N(Rₐ)—R₂;
   (d) when A is 3-(m-tolyl)-pyrazol-1-yl, and X is —CH—, Y is —C—, and Z is —O—, and B is —(CO)-(6-12 membered heterocycloalkyl), said heterocycloalkyl in group B is not unsubstituted morpholine, unsubstituted piperidine, unsubstituted pyrrolidine, unsubstituted piperazine, or 4-methylpiperazine;
   (e) when W is —N—, X is —N—, —NH—, or —NCH₃—, or Z is —N—, —NH—, or —NCH₃—, and Y is —C—, then B is not pyridyl, unsubstituted pyrrolidinyl, unsubstituted piperidinyl, 1-methyl-4-piperidinyl, 2-methyl-4-piperidinyl, tetrahydropyranyl, dihydropyranyl, 1-tert-butoxycarbonyl-4-azetidinyl, 1-methyl-4-azetidinyl, 4-azetidinyl, 1,2-dihydroxy-1-ethyl;
   (f) when W is —CH—, X is —CH— and Y is —N—, B is not 2-(1-methyl-pyrazol-3-yl)ethyl;
   (g) when B is pyrid-4-yl, R₁ is morpholin-4-yl, X is —CH—, Y is —C—, and Z is —S—, A is not 3-(3-methylphenyl)-5-hydroxy-pyrazol-1-yl, 3-(3-methoxyphenyl)-5-hydroxy-pyrazol-1-yl, or 3-(3-isopropoxyphenyl)-5-hydroxy-pyrazol-1-yl; and
   (h) when B is phenyl, R₁ is N-cyclohexylamino, X is —O—, Y is —C—, and Z is —CH—, A is not 3,5-diphenyl-pyrazol-1-yl, 3-methyl-5-phenyl-pyrazol-1-yl, 3-trifluoromethyl-5-phenyl-pyrazol-1-yl, or 3,5-dimethyl-4-phenyl-pyrazol-1-yl;
   (i) when W is —N—, X is —CH— or —C(Me)-, Y is —C—, Z is —S— or —O—, and R₁ is 4-morpholinyl, then A is not 1H-indazol-4-yl, 1H-indol-4-yl, 1H-pyrrolo[2,3-b]pyridin-5-yl, or 2-methyl-3H-imidazo[4,5]pyridin-6-yl;
   (j) when W is —N—, X is —S— or —O—, Y is —C—, Z is —CH— or —C(Me)-, and R₁ is 4-morpholinyl, then A is not 1H-indazol-4-yl, 1H-indol-4-yl, 1H-pyrrolo[2,3-b]pyridin-5-yl, or 2-methyl-3H-imidazo[4,5]pyridin-6-yl;
   (k) when W is —N—, X is —CH— or —C(Me)-, Y is —C—, Z is —S— or —O—, B is H or optionally substituted C₁₋₆alkyl, and R₁ is 4-morpholinyl, then A is not 2-oxoindolin-4-yl, 1-acetylindolin-4-yl, 1H-indazol-6-yl, 1H-indol-5-yl, 1H-indol-6-yl, or quinolin-3-yl;
   (l) when W is —N—, X is —S— or —O—, Y is —C—, Z is —CH— or —C(Me)-, B is H or optionally substituted C₁₋₆alkyl, and R₁ is 4-morpholinyl, then A is not 2-oxoindolin-4-yl, 1-acetylindolin-4-yl, 1H-indazol-6-yl, 1H-indol-5-yl, 1H-indol-6-yl, or quinolin-3-yl;
   (m) when W is —N—, X is —S—, Z is —N—, Y is —C—, and R₁ is 4-morpholinyl, then A is not optionally substituted pyrid-3-yl, optionally substituted pyrimidin-5-yl, unsubstituted quinolin-3-yl, unsubstituted 1H-indazol-4-yl, unsubstituted 1H-indol-4-yl, or unsubstituted 1H-pyrrolo[2,3-b]pyridin-5-yl;
   (n) when W is —N—, X is —N—, Z is —S—, Y is —C—, and R₁ is 4-morpholinyl, then A is not optionally substituted pyrid-3-yl, optionally substituted pyrimidin-5-yl, unsubstituted quinolin-3-yl, unsubstituted 1H-indazol-4-yl, unsubstituted 1H-indol-4-yl, or unsubstituted 1H-pyrrolo[2,3-b]pyridin-5-yl,
   (o) when W is —N—, X is —S—, Z is —N—, Y is —C—, and A is unsubstituted indolyl, pyrazolyl, imidazolyl, or triazolyl, then R₁ is not 3-methylmorpholin-4-yl
   (p) when W is —N—, X is —NR₃—, R₃ is selected from methyl, ethyl, isopropyl, and cyclopentyl, Z is —N—, Y is —C—, and R₁ is 4-morpholinyl, then A is not benzo[d][1,3]dioxol-5-yl, pyrimidin-5-yl, 2-aminopyrimidin-5-yl, pyridin-3-yl, 6-aminopyridin-3-yl, 6-methoxypyridin-3-yl, quinolin-3-yl, or pyridin-4-yl;
   (q) when W is —N—, X is —NR₃—, R₃ is 1-benzylpiperidin-4-yl, Z is —CH—, Y is —N—, and R₁ is 4-morpholinyl, then A is not pyridin-3-yl, pyridin-4-yl, quinolin-3-yl, quinolin-6-yl, 1H-indol-2-yl, 1H-indol-5-yl, 1H-indol-6-yl, 1H-indol-7-yl, 1-methyl-indol-5-yl, or 2-oxo-2,3-dihydro-1H-benzo[d]imidazol-5-yl;
   (r) when W is —N—, X is —CH— or —C(Me)-, Y is —C—, Z is —S— or —O—, and R₁ is 4-morpholinyl, then A is not 2-aminopyrimidin-5-yl;
(s) when W is —N—, Y is —C—, and R₁ is 4-morpholinyl, A is not 2-methyl-1H-benzo[d]imidazole-1-yl or 2-ethyl-1H-benzo[d]imidazol-1-yl;
   (t) when W is —N—, X is —NR₃—, R₃ is methyl, Z is —N—, Y is —C—, and B is optionally substituted —CH₂-piperidine, optionally substituted —CH₂-piperazine, —CH₂-azetidine, or 2,5-diazabicyclo[2.2.1]heptan-2-yl, then A is not 2-methyl-1H-benzo[d]imidazol-1-yl or 2-ethyl-1H-benzo[d]imidazol-1-yl;
   (u) when W is —N—, X is —CH—, Y is —C—, Z is —S—, and R₁ is 4-morpholinyl, then A is not 5-fluoro-1H-indol-4-yl, 6-fluoro-1H-indol-4-yl, 6-cyano-1H-indol-4-yl, 5,7-difluoro-1H-indol-4-yl, 2-methyl-1H-indol-4-yl, 1-methyl-1H-indol-4-yl, 1H-pyrrolo[2,3-c]pyridin-4-yl, 1H-pyrrolo[2,3-b]pyridin-4-yl, 2-oxo-1H-indolin-4-yl, imidazo[1,2-a]pyridin-5-yl, 2-methyl-quinolin-5-yl, 2-methylbenzo[b]thiophen-3-yl, 1H-indazol-3-yl, 1H-indol-3-yl, 1H-benzo[d]imidazole-1-yl, 1H-indazol-1-yl, 1H-indol-1-yl, 2-methyl-benzo[d]imidazole-1-yl, or 3-methylbenzo[d]imidazole-1-yl;
   (v) when W is —N—, X is —CH— or —C(Me)-, Y is —C—, Z is —S— or —O—, and R₁ is 4-morpholinyl, then A is not optionally substituted 1H-benzo[d]imidazol-1-yl, optionally substituted quinolin-5-yl, optionally substituted 1H-pyrrolo[2,3-c]pyridin-4-yl, optionally substituted 1H-pyrrolo[2,3-c]pyridin-3-yl, optionally substituted 1H-pyrrolo[3,2-c]pyridin-4-yl, optionally substituted isoquinolin-8-yl, optionally substituted isoquinolin-4-yl, optionally substituted isoquinolin-5-yl, optionally substituted 1H-indazol-1-yl, optionally substituted 1H-indazol-3-yl, optionally substituted benzofuran-3-yl, optionally substituted 1H-pyrrolo[2,3-b]pyridin-3-yl, optionally substituted 1H-pyrrolo[2,3-b]pyridin-5-yl, optionally substituted 1H-pyrrolo[2,3-b]pyridin-4-yl, optionally substituted benzo[b]thiophen-3-yl, optionally substituted 1H-pyrolo[3,2-c]-pyridin-1-yl, 2-oxo-1H-benzo[d]imidazol-1-yl, optionally substituted 1H-benzo[d]imidazol-1-yl, optionally substituted 2H-indazol-3-yl, 2-oxo-1-indolin-1-yl, optionally substituted 2H-indazol-3-yl, optionally substituted imidazo[3,2-d]pyrimidin-6-yl, optionally substituted imidazo[1,5-a]pyridin-8-yl, optionally substituted imidazo[1,2-a]pyridin-5-yl, optionally substituted imidazo[4,5-c]pyridin-1-yl, optionally substituted imidazo[4,5-c]pyridin-3-yl, 1-oxo-1,2-dihydroisoquinolin-4-yl, 2-methyl-3-oxo-2,3-dihydro-1H-indazol-1-yl, [1,2,4]-triazolo[4,3-a]pyridin-5-yl, cinnolin-4-yl, or benzo[d]isothiazol-3-yl;
   (w) when W is —N—, X is —S—, —NH— or —N(Me)-, Y is —C—, Z is —N—, and R₁ is 4-morpholinyl, then A is not optionally substituted 1H-benzo[d]imidazol-1-yl, optionally substituted quinolin-5-yl, optionally substituted 1H-pyrrolo[2,3-c]pyridin-4-yl, optionally substituted 1H-pyrrolo[2,3-c]pyridin-3-yl, optionally substituted 1H-pyrrolo[3,2-c]pyridin-4-yl, optionally substituted isoquinolin-8-yl, optionally substituted isoquinolin-4-yl, optionally substituted isoquinolin-5-yl, optionally substituted 1H-indazol-1-yl, optionally substituted 1H-indazol-3-yl, optionally substituted benzofuran-3-yl, optionally substituted 1H-pyrrolo[2,3-b]pyridin-3-yl, optionally substituted 1H-pyrrolo[2,3-b]pyridin-5-yl, optionally substituted 1H-pyrrolo[2,3-b]pyridin-4-yl, optionally substituted benzo[b]thiophen-3-yl, optionally substituted 1H-pyrolo[3,2-c]-pyridin-1-yl, 2-oxo-1H-benzo[d]imidazol-1-yl, optionally substituted 1H-benzo[d]imidazol-1-yl, optionally substituted 2H-indazol-3-yl, 2-oxo-1-indolin-1-yl, optionally substituted 2H-indazol-3-yl, optionally substituted imidazo[3,2-d]pyrimidin-6-yl, optionally substituted imidazo[1,5-a]pyridin-8-yl, optionally substituted imidazo[1,2-a]pyridin-5-yl, optionally substituted imidazo[4,5-c]pyridin-1-yl, optionally substituted imidazo[4,5-c]pyridin-3-yl, 1-oxo-1,2-dihydroisoquinolin-4-yl, 2-methyl-3-oxo-2,3-dihydro-1H-indazol-1-yl, [1,2,4]-triazolo[4,3-a]pyridin-5-yl, cinnolin-4-yl, or benzo[d]isothiazol-3-yl;
   (x) when W is —N—, X is —NH—, Y is —C—, Z is —S—, and R₁ is 4-morpholinyl, then A is not optionally substituted 1H-benzo[d]imidazol-1-yl, optionally substituted quinolin-5-yl, optionally substituted 1H-pyrrolo[2,3-c]pyridin-4-yl, optionally substituted 1H-pyrrolo[2,3-c]pyridin-3-yl, optionally substituted 1H-pyrrolo[3,2-c]pyridin-4-yl, optionally substituted isoquinolin-8-yl, optionally substituted isoquinolin-4-yl, optionally substituted isoquinolin-5-yl, optionally substituted 1H-indazol-1-yl, optionally substituted 1H-indazol-3-yl, optionally substituted benzofuran-3-yl, optionally substituted 1H-pyrrolo[2,3-b]pyridin-3-yl, optionally substituted 1H-pyrrolo[2,3-b]pyridin-5-yl, optionally substituted 1H-pyrrolo[2,3-b]pyridin-4-yl, optionally substituted benzo[b]thiophen-3-yl, optionally substituted 1H-pyrolo[3,2-c]-pyridin-1-yl, 2-oxo-1H-benzo[d]imidazol-1-yl, optionally substituted 1H-benzo[d]imidazol-1-yl, optionally substituted 2H-indazol-3-yl, 2-oxo-1-indolin-1-yl, optionally substituted 2H-indazol-3-yl, optionally substituted imidazo[3,2-d]pyrimidin-6-yl, optionally substituted imidazo[1,5-a]pyridin-8-yl, optionally substituted imidazo[1,2-a]pyridin-5-yl, optionally substituted imidazo[4,5-c]pyridin-1-yl, optionally substituted imidazo[4,5-c]pyridin-3-yl, 1-oxo-1,2-dihydroisoquinolin-4-yl, 2-methyl-3-oxo-2,3-dihydro-1H-indazol-1-yl, [1,2,4]-triazolo[4,3-a]pyridin-5-yl, cinnolin-4-yl, or benzo[d]isothiazol-3-yl;
   (y) when W is —N—, X is —N—, —NH— or —N(CH₂CH₂OH)—, Z is —N—, Y is —C—, and R₁ is 4-morpholinyl, then A is not 1H-indol-4-yl or 5-fluoro-1H-indol-4-yl;
   (z) when W is —N— or —CH—, X is —NH—, —N(Me)-, —N(Et)-, or —N(CH₂-cyclopropyl)-, Y is —C—, Z is —N—, and R₁ is 4-morpholinyl, then A is not an unsubstituted pyrazol-4-yl or a pyrazol-4-yl substituted by one or more groups selected from methyl, ethyl, n-propyl, isopropyl, and trifluoromethyl;
   (aa) when W is —N—, X is —NH—, Y is —C—, Z is —CH— or —N—, and R₁ is 4-morpholinyl, 3-methylmorpholin-4-yl, 3,3-dimethylmorpholin-4-yl, 2-oxa-5-azabicyclo[2.2.1]hept-5-yl, or 3-oxa-8-azabicyclo[3.2.1]oct-8-yl, then A is not 4-morpholinyl, 3-methylmorpholin-4-yl, piperidin-1-yl, 4-hydroxypiperidin-1-yl, 4-methoxypiperidin-1-yl, 4,4-difluoropiperidin-1-yl, or 8-oxa-3-azabicyclo[3.2.1]oct-3-yl;
   (bb) when W is —CH—, X is —S—, Y is —C—, and X is —CH—, B is not pyrazol-5-yl when A is pyrrolidine-1-yl;
      and wherein the compound of Formula I is not
4-(2-methyl-5-(3-(m-tolyl)-1H-pyrazol-1-yl)-1H-pyrrolo[3,2-b]pyridin-7-yl)morpholine,
2-(2-(2-aminopyrimidin-5-yl)-9-(2-hydroxyethyl)-6-morpholino-9H-purin-8-yl)propan-2-ol,
1-ethyl-3-(5-(6-(3-ethylmorpholino)-7-methyl-7H-purin-2-yl)pyrimidin-2-yl)urea, or
5-pyrrolidino-2-(4-methoxyphenyl)-2H-[1,2,3]triazolo[4,5-d]pyrimidin-7-one or a tautomer thereof.

In particular embodiments, the invention provides a compound according to the following Formulas
1.1 The compound of Formula I, wherein X is —S—, Z is —CH— or —CR₃—, and Y is —C—;
1.2 The compound of Formula I, wherein X is —CH— or —CR₃—, Z is —S—, and Y is —C—;
1.3 The compound of Formula I, wherein X is —O—, Z is —CH— or —CR₃—, and Y is —C—;
1.4 The compound of Formula I, wherein X is —CH— or —CR₃—, Z is —O—, and Y is —C—;
1.5 The compound of Formula I, wherein X is —NH—, Z is —CH— or —CR₃—, and Y is —C—;
1.6 The compound of Formula I, wherein X is —CH— or —CR₃—, Z is —NH—, and Y is —C—;
1.7 The compound of Formula I, wherein X is —NR₃—, Z is —CH— or —CR₃—, and Y is —C—;
1.8 The compound of Formula I, wherein X is —CH— or —CR₃—, Z is —NR₃—, and Y is —C—;
1.9 The compound of Formula I, wherein X and Z are —CH— or —CR₃—, and Y is —N—;
1.10 The compound of Formula I, wherein X is —S—, Z is —N—, and Y is —C—;
1.11 The compound of Formula I, wherein X is —N—, Z is —S—, and Y is —C—;
1.12 The compound of Formula I, wherein X is —O—, Z is —N—, and Y is —C—;
1.13 The compound of Formula I, wherein X is —N—, Z is —O—, and Y is —C—;
1.14 The compound of Formula I, wherein X is —NH—, Z is —N—, and Y is —C—;
1.15 The compound of Formula I, wherein X is —N—, Z is —NH—, and Y is —C—;
1.16 The compound of Formula I, wherein X is —NR₃—, Z is —N—, and Y is —C—;
1.17 The compound of Formula I, wherein X is —N—, Z is —NR₃—, and Y is —C—;
1.18 The compound of Formula I, wherein X is —N—, Z is —CH— or —CR₃—, and Y is —N—;
1.19 The compound of Formula I, wherein X is —CH— or —CR₃—, Z is —N—, and Y is —N—;
1.20 The compound of Formula I, wherein X and Z are —N—, and Y is —N—;
1.21 The compound of Formula I or any of 1.1-1.20, wherein X or Z is —CH—;
1.22 The compound of Formula I, or any of 1.1-1.21, wherein R₃ is H;
1.23 The compound of Formula I, or any of 1.1-1.21, wherein R₃ is C₁₋₆alkyl (e.g., methyl);
1.24 The compound of Formula I or any of 1.1-1.23, wherein W is —CH— or —CR₄—, optionally wherein R₄ is fluoro, —OH, —NH₂, methyl, CF₃, methoxy, methylamino, or dimethylamino;
1.25 The compound of Formula I or any of 1.1-1.23, wherein W is —N—;
1.26 The compound of Formula I, wherein the compound of Formula I has a core structure selected from any of the following:

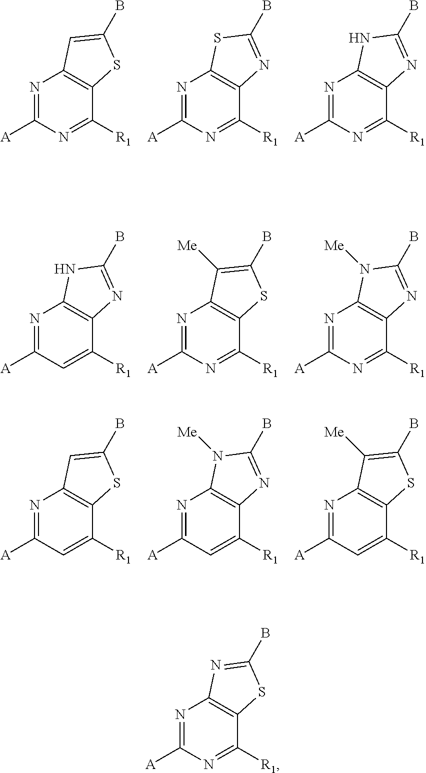

wherein A, B and R₁ are as defined for the Compound of Formula I above;
1.27 The compound of Formula I, wherein the compound of Formula I has a core structure selected from any of the following:

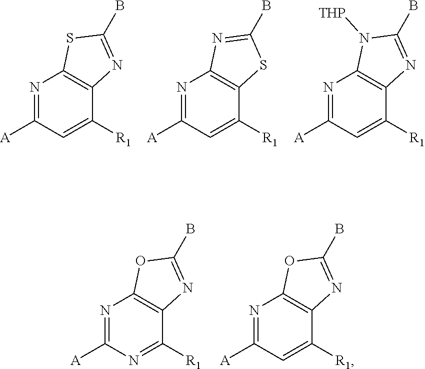

wherein A, B and R₁ are as defined for the Compound of Formula I above;
1.28 The compound of Formula I, wherein the compound of Formula I has a core structure selected from any of the following:

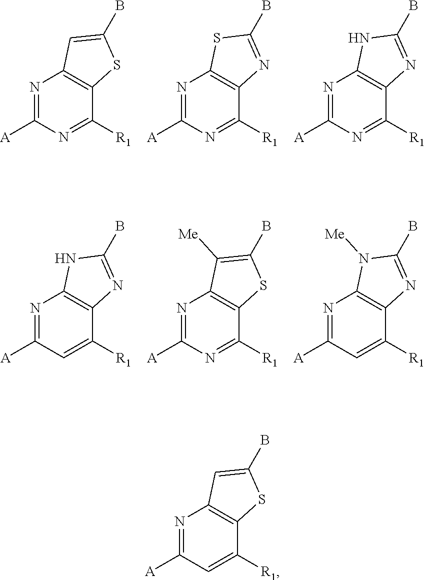

wherein A, B and R₁ are as defined for the Compound of Formula I above;
1.29 The compound of Formula I, or any of 1.1-1.28, wherein A is an optionally substituted heteroaryl;
1.30 The compound of Formula 1.29, wherein A is a heteroaryl selected from pyridine, pyrimidine, pyridazine, pyrazine, triazine, indole, benzimidazole, benzoxazole, benzothiazole, indazole, benzisoxazole, and benzisothiazole;
1.31 The compound of Formula 1.29, wherein A is a 5-membered heteroaryl, e.g., selected from thiophene, furan, pyrrole, oxazole, imidazole, thiazole, pyrazole, isoxazole, isothiazole, triazole (e.g., 1,2,3-triazole, or 1,2,4-triazole), oxadiazole (e.g., 1,2,3-oxadiazole, or 1,2,4-oxadiazole), thiadiazole (e.g., 1,2,3-thiadiazole, or 1,2,4-thiadiazole), and tetrazole (e.g., 1,2,3,4-tetrazole);
1.32 The compound of Formula 1.31, wherein said heteroaryl is selected from oxazole, imidazole, thiazole, pyrazole, isoxazole, isothiazole, and triazole (e.g., 1,2,3-triazole);
1.33 The compound of Formula 1.32, wherein said heteroaryl is pyrazole (e.g., 3-substituted-pyrazol-1-yl, 1-substituted-pyrazol-3-yl, 2-substituted-pyrazol-4-yl, 4-substituted-pyrazol-1-yl, 1-substituted-pyrazol-5-yl, or 5-substituted-pyrazol-3-yl);
1.34 The compound of Formula 1.33, wherein said heteroaryl is 3-substituted-pyrazol-1-yl;
1.35 The compound of Formula 1.32, wherein said heteroaryl is thiazole (e.g., 2-substituted-thiazol-4-yl, 2-substituted-thiazol-5-yl, 4-substituted-thiazol-2-yl, or 5-substituted-thiazol-2-yl);
1.36 The compound of Formula 1.32, wherein said heteroaryl is oxazole (e.g., 2-substituted-oxazol-4-yl, 2-substituted-oxazol-5-yl, 4-substituted-oxazol-2-yl, or 5-substituted-oxazol-2-yl);
1.37 The compound of Formula 1.32, wherein said heteroaryl is imidazole (e.g., 2-substituted-imidazol-4-yl, 4-substituted-imidazol-2-yl, 2-substituted-imidazol-5-yl, 5-substituted-imidazol-2-yl, 4-substituted-imidazol-1-yl, or 1-substituted-imidazol-4-yl);
1.38 The compound of Formula 1.32, wherein said heteroaryl is triazole (e.g., 4-substituted-1,2,3-triazol-2-yl or 2-substituted-1,2,3-triazol-4-yl);
1.39 Any of Compounds 1.23-1.38, wherein said heteroaryl is substituted with one or more groups selected from OH, CN, C₁₋₆alkyl (e.g., methyl), halogen (e.g., F), C₁₋₆alkoxy (e.g., methoxy), haloC₁₋₆alkyl (e.g., CF₃), carboxy (COOH), aryl, heteroaryl, C₃₋₆cycloalkyl, and 3- to 10-membered heterocycloalkyl or heterocycloalkenyl (e.g., 3- to 6-membered heterocycloalkyl or heterocycloalkenyl, such as 1-piperidinyl, 3-piperidinyl, 1,2,3,6-tetrahydropyridin-1-yl or 1,2,3,6-tetrahydropyridin-3-yl), wherein said alkyl, alkoxy, aryl, heteroaryl, cycloalkyl, heterocycloalkyl, or heterocycloalkenyl, is each optionally independently substituted with one or more groups selected from OH, CN, C₁₋₆alkyl (e.g., methyl or t-butyl), halogen (e.g., F or Br), C₁₋₆alkoxy (e.g., methoxy, ethoxy), haloC₁₋₆alkyl (e.g., CF₃), haloC₁₋₆alkoxy (e.g., OCF₃), carboxy (COOH), C₃₋₆cycloalkyl, and 5- or 6-membered heterocycloalkyl, and wherein said alkyl, alkoxy, cycloalkyl, or heterocycloalkyl, is each optionally independently substituted with one or more groups selected from OH, CN, C₁₋₆alkyl (e.g., methyl), halogen (e.g., F or Br), C₁₋₆alkoxy (e.g., methoxy), haloC₁₋₆alkyl (e.g., CF₃), haloC₁₋₆alkoxy (e.g., OCF₃), and C₃₋₆cycloalkyl;
1.40 The compound of Formula 1.39, wherein said heteroaryl is substituted with a 5- or 6-membered heterocycloalkyl or heterocycloalkenyl, e.g., selected from piperidinyl (e.g., piperidin-1-yl, or piperidin-3-yl) and 1,2,3,6-tetrahydropyridine (e.g., 1,2,3,6-tetrahydropyridin-1-yl or 1,2,3,6-tetrahydropyridin-3-yl);
1.41 The compound of Formula 1.39, wherein said heteroaryl is substituted with aryl (e.g., phenyl) or heteroaryl (e.g., pyridyl or pyrimidinyl), wherein said aryl or heteroaryl is optionally substituted with one or more groups selected from OH, CN, C₁₋₆alkyl (e.g., methyl or t-butyl), halogen (e.g., F or Br), C₁₋₆alkoxy (e.g., methoxy or ethoxy), haloC₁₋₆alkoxy (e.g., OCF₃, OCHF₂), haloC₁₋₆alkyl (e.g., CF₃, CHF₂), hydroxyC₁₋₆alkyl (e.g., hydroxymethyl, 1-hydroxyethyl), C₁₋₆alkoxyC₁₋₆alkyl (e.g., methoxymethyl, 1-methoxyethyl), and —C(O)—C₁₋₆alkyl (e.g., acetyl);
1.42 The compound of Formula 1.39, wherein said heteroaryl is substituted with an optionally substituted heteroaryl selected from pyridyl, pyrimidinyl, pyrazinyl, pyridazinyl, indolyl (e.g., indol-1-yl, or indol-3-yl), indazolyl (e.g., indazol-1-yl, or indazol-3-yl), benzimidazolyl (e.g., benzimidazol-1-yl, or benzimidazol-2-yl), benzisoxazolyl (e.g., benzisoxazol-3-yl), benzisothiazolyl (e.g., benzisothiazol-3-yl), benzoxazolyl (e.g., benzoxazol-2-yl), benzothiazolyl (e.g., benzothiazol-2-yl);
1.43 The compound of Formula 1.39, wherein said heteroaryl is substituted with unsubstituted phenyl or C₁₋₆alkyl (e.g., methyl or t-butyl);
1.44 The compound of Formula 1.39, wherein said heteroaryl is substituted with phenyl substituted with one, two or three groups independently selected from OH, CN, C₁₋₆alkyl (e.g., methyl or t-butyl), halogen (e.g., F or Br), C₁₋₆alkoxy (e.g., methoxy or ethoxy), haloC₁₋₆alkoxy (e.g., OCF₃, OCHF₂), haloC₁₋₆alkyl (e.g., CF₃, CHF₂), C₃₋₆cycloalkyl, —C(O)—C₁₋₆alkyl (e.g., acetyl), and 5- or 6-membered heterocycloalkyl, and wherein said alkyl, alkoxy, cycloalkyl, or heterocycloalkyl, is each optionally independently substituted with one or more groups selected from OH, CN, C₁₋₆alkyl (e.g., methyl), halogen (e.g., F or Br), C₁₋₆alkoxy (e.g., methoxy), haloC₁₋₆alkyl (e.g., CF₃), haloC₁₋₆alkoxy (e.g., OCF₃), and C₃₋₆cycloalkyl;
1.45 The compound Formula 1.44, wherein said heteroaryl is 3-substituted-pyrazol-1-yl, and said substituent is phenyl substituted with one, two or three groups independently selected from CN, C₁₋₆alkyl (e.g., methyl or t-butyl), halogen (e.g., F or Br), C₁₋₆alkoxy (e.g., methoxy or ethoxy), haloC₁₋₆alkoxy (e.g., OCF₃), haloC₁₋₆alkyl (e.g., CF₃), C₃₋₆cycloalkyl, and 5- or 6-membered heterocycloalkyl, and wherein said alkyl, alkoxy, cycloalkyl, or heterocycloalkyl, is each optionally independently substituted with one or more groups selected from OH, CN, C₁₋₆alkyl (e.g., methyl), halogen (e.g., F or Br), C₁₋₆alkoxy (e.g., methoxy), haloC₁₋₆alkyl (e.g., CF₃), haloC₁₋₆alkoxy (e.g., OCF₃), and C₃₋₆cycloalkyl;
1.46 The compound Formula 1.44, wherein said heteroaryl is 3-substituted-pyrazol-1-yl, and said substituent is phenyl substituted with one group selected from CN, C₁₋₆alkyl (e.g., methyl or t-butyl), halogen (e.g., F or Br), C₁₋₆alkoxy (e.g., methoxy or ethoxy), haloC₁₋₆alkoxy (e.g., OCF₃), and haloC₁₋₆alkyl (e.g., CF₃), wherein said alkyl or alkoxy is optionally substituted with one or more groups selected from OH, CN, C₁₋₆alkyl (e.g., methyl), and C₁₋₆alkoxy (e.g., methoxy);
1.47 The compound of Formula 1.46, wherein said phenyl is meta-substituted;
1.48 The compound of Formula 1.46 or 1.47, wherein said phenyl is substituted with one group selected from methyl, bromo, methoxy, ethoxy, trifluoromethyl, trifluoromethoxy, hydroxymethyl, methoxymethyl, 1-hydroxyethyl, and 1-methoxyethyl;
1.49 The compound Formula 1.44, wherein said heteroaryl is 2-substituted-thiazol-4-yl, 2-substituted imidazole-4-yl, 2-substituted oxazol-4-yl, 4-substituted imidazole-2-yl, 4-substituted-1,2,3-triazol-2-yl, or 2-substituted-1,2,3-triazol-4-yl, and said substituent is phenyl substituted with one group selected from CN, C₁₋₆alkyl (e.g., methyl or t-butyl), halogen (e.g., F or Br), C₁₋₆alkoxy (e.g., methoxy or ethoxy), haloC₁₋₆alkoxy (e.g., OCF₃), and haloC₁₋₆alkyl (e.g., CF₃), wherein said alkyl or alkoxy is optionally substituted with one or more groups selected from OH, CN, C₁₋₆alkyl (e.g., methyl), and C₁₋₆alkoxy (e.g., methoxy);
1.50 The compound of Formula 1.49, wherein said phenyl is meta-substituted;
1.51 The compound of Formula 1.49 or 1.50, wherein said phenyl is substituted with one group selected from methyl, bromo, chloro, fluoro, methoxy, ethoxy, trifluoromethyl, difluoromethyl, trifluoromethoxy, difluoromethoxy, hydroxymethyl, methoxymethyl, 1-hydroxyethyl, 1-methoxyethyl, and acetyl;
1.52 The compound of Formula 1.51, wherein said phenyl is substituted with methyl;
1.53 The compound of any of Formulas 1.37-1.48, wherein said heteroaryl is an imidazolyl, and in addition to being substituted with said heterocycloalkyl, heterocycloalkenyl, aryl (e.g., phenyl) or heteroaryl, said imidazolyl is also N-substituted with a C₁₋₆alkyl (e.g., methyl);
1.54 The compound of Formula 1.29, wherein substituent A is selected from the following:

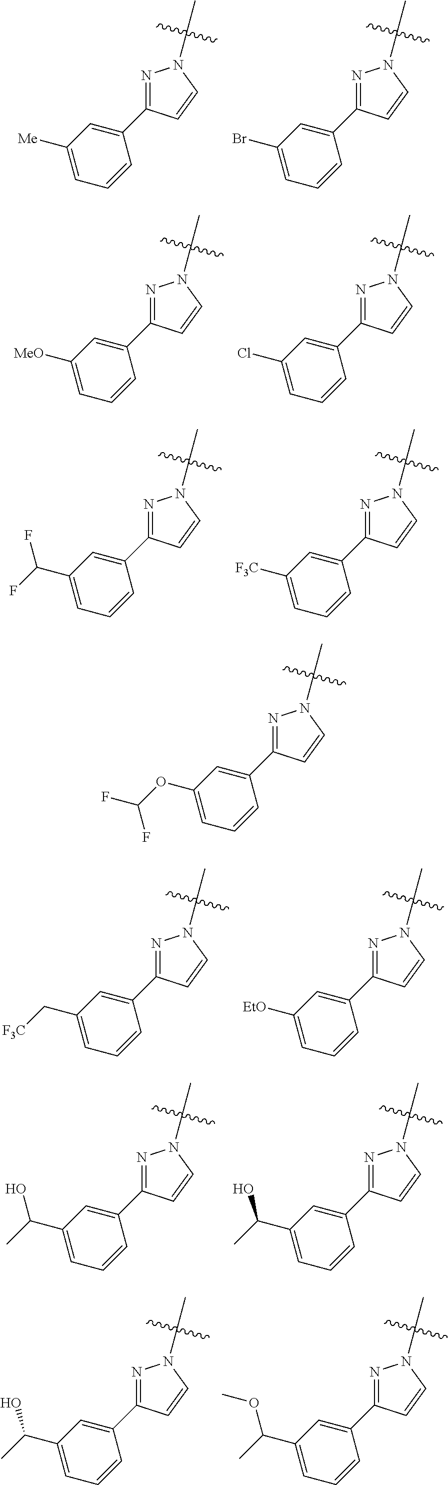

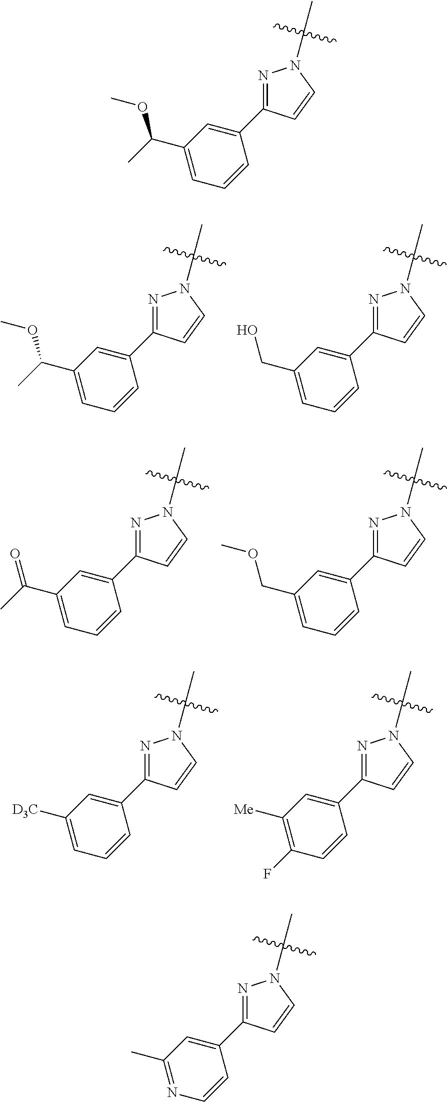

1.55 The compound of formula 1.29, wherein substituent A is selected from:

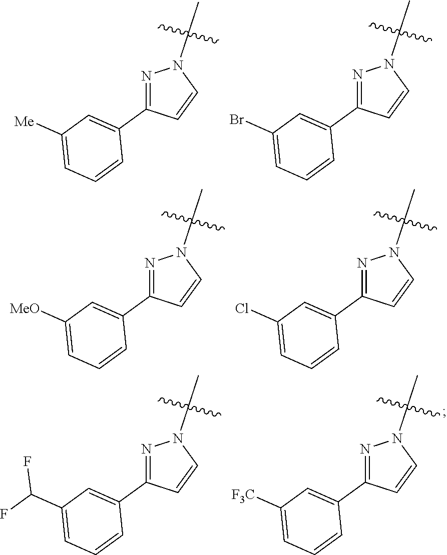 [SMILES: *n1ccc(-c2cccc(C)c2)n1 ; *n1ccc(-c2cccc(Br)c2)n1 ; *n1ccc(-c2cccc(OC)c2)n1 ; *n1ccc(-c2cccc(Cl)c2)n1 ; *n1ccc(-c2cccc(C(F)F)c2)n1 ; *n1ccc(-c2cccc(C(F)(F)F)c2)n1]

1.56 The compound of Formula 1.29, wherein substituent A is selected from:

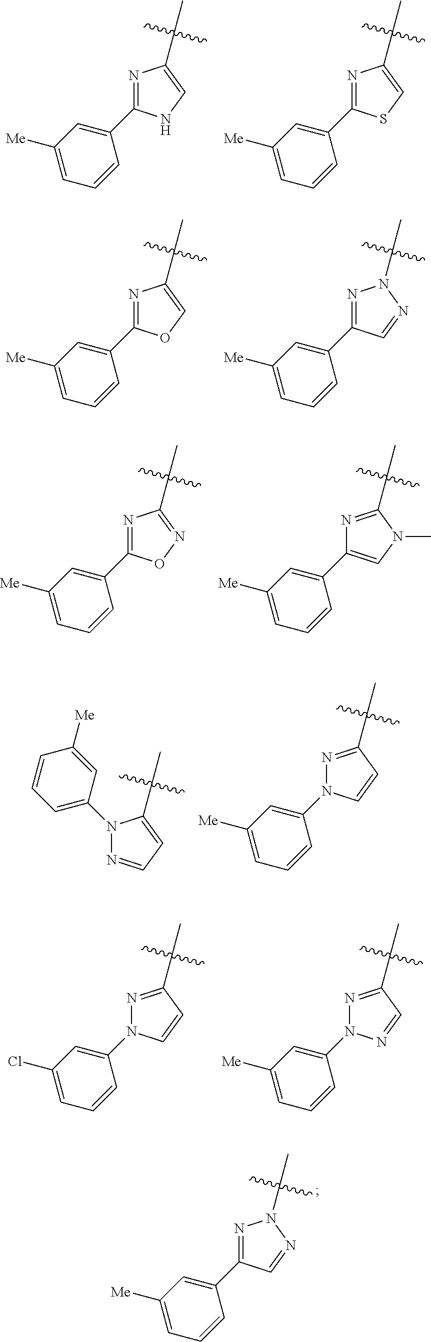

1.57 The compound of Formula I or any of 1.1-1.56, wherein A is an optionally substituted heterocycloalkyl;
1.58 The compound of Formula 1.57, wherein said heterocycloalkyl is selected from aziridine, azetidine, oxetane, pyrrolidine, tetrahydrofuran, tetrahydropyran, morpholine, piperidine, and piperazine;
1.59 The compound of Formula 1.57, wherein said heterocycloalkyl is pyrrolidine;
1.60 Any of Compounds 1.57-1.60, wherein said heterocycloalkyl is substituted with one or more groups selected from OH, CN, C₁₋₆alkyl (e.g., methyl), halogen (e.g., F), C₁₋₆alkoxy (e.g., methoxy), haloC₁₋₆alkyl (e.g., CF₃), carboxy (COOH), aryl, heteroaryl, C₃₋₆cycloalkyl, and 3- to 6-membered heterocycloalkyl, wherein said alkyl, alkoxy, aryl, heteroaryl, cycloalkyl, and heterocycloalkyl is each optionally independently substituted with one or more groups selected from OH, CN, C₁₋₆alkyl (e.g., methyl or t-butyl), halogen (e.g., F or Br), C₁₋₆alkoxy (e.g., methoxy), haloC₁₋₆alkyl (e.g., CF₃), carboxy (COOH), C₃₋₆cycloalkyl, and 5- or 6-membered heterocycloalkyl;
1.61 The compound of Formula 1.60, wherein said heterocycloalkyl is substituted with aryl (e.g., phenyl) or heteroaryl (e.g., pyridyl or pyrimidinyl), wherein said aryl or heteroaryl is optionally substituted with one or more groups selected from OH, CN, C₁₋₆alkyl (e.g., methyl or t-butyl), halogen (e.g., F or Br), C₁₋₆alkoxy (e.g., methoxy), and haloC₁₋₆alkyl (e.g., CF₃);
1.62 The compound of Formula 1.61, wherein said heterocycloalkyl is substituted with unsubstituted phenyl;
1.63 The compound of Formula 1.61, wherein said heterocycloalkyl is substituted with phenyl substituted with one, two or three groups independently selected from CN, C₁₋₆alkyl (e.g., methyl or t-butyl), halogen (e.g., F or Br), C₁₋₆alkoxy (e.g., methoxy), and haloC₁₋₆alkyl (e.g., CF₃)
1.64 The compound of Formula I, or any of 1.1-1.63, wherein B is optionally substituted aryl or optionally substituted heteroaryl;
1.65 The compound of Formula 1.64, wherein B is selected from halo (e.g., bromo), phenyl, pyridine, pyrimidine, pyridazine, pyrazine, triazine, tetrazine, thiophene, furan, pyrrole, oxazole, imidazole, thiazole, pyrazole, isoxazole, isothiazole, indole, indazole, benzimidazole, benzisoxazole, benzisothiazole, benzoxazole, and benzothiazole, each optionally an N-oxide thereof (e.g., pyridyl-N-oxide), and each optionally substituted;
1.66 The compound of Formula 1.64, wherein B is selected from phenyl, pyridine, pyrimidine, pyridazine, and pyrazine, each optionally substituted;
1.67 The compound of Formula 1.64, wherein B is selected from pyridine and pyrimidine, each optionally substituted;
1.68 The compound of Formula 1.64, wherein B is optionally substituted pyrazole (e.g., 1-substituted 4-pyrazolyl);
1.69 The compound of Formula 1.67, wherein B is pyridine, e.g., 2-pyridinyl, 3-pyridinyl, or 4-pyridinyl, or 4-pyridinyl-N-oxide, each optionally substituted;
1.70 The compound of Formula 1.64, wherein B is pyrazole substituted with C₁₋₆alkyl, e.g., 1-methyl-4-pyrazolyl;
1.71 The compound of Formula I, or any of 1.1-1.63, wherein B is optionally substituted heterocycloalkyl or heterocycloalkenyl;
1.72 The compound of Formula 1.64, wherein B is selected from morpholine, piperidine, 1,2,3,6-tetrahydropyridinyl, piperazine, tetrahydropyran, pyrrolidine, tetrahydrofuran, oxetane, azetidine, oxirane, and aziridine, e.g., each optionally substituted with halo (e.g., F), C₁₋₆alkyl (e.g., N-substituted, e.g., N-methyl or N-isopropyl), C₁₋₆alkoxy (e.g., methoxy), —N(C₁₋₆alkyl)(C₁₋₆alkyl) (e.g., dimethylamino), or —SO₂—C₁₋₆alkyl (e.g., methylsulfonyl);
1.73 The compound of Formula 1.72, wherein B is selected from 3-methoxy-3-oxetanyl, 4-(dimethylamino)-1-piperidinyl, 3-(dimethylamino)-1-pyrrolidinyl, 4-methyl-1-piperazinyl, 4-isopropyl-1-piperazinyl, 4-morpholinyl, and 4-(methylsulfonyl)-1-piperazinyl;
1.74 The compound of Formula I, or any of 1.1-1.63, wherein B is optionally substituted —CH₂—(3- to 6-membered heterocycloalkyl);
1.75 The compound of Formula 1.74, wherein B is —CH₂—(3- to 6-membered heterocycloalkyl) and said heterocycloalkyl is selected from morpholine, piperidine, piperazine, tetrahydropyran, pyrrolidine, tetrahydrofuran, oxetane, azetidine, oxirane, and aziridine, each optionally substituted, for example, by halo (e.g., F), C₁₋₆alkyl (e.g., methyl, isopropyl), C₃₋₆cycloalkyl (e.g., cyclopropyl, cyclobutyl), —NH(C₁₋₆alkyl) (e.g., N-methylamino, N-methylcyclopropylamino), —NH(C₃₋₆cycloalkyl), (e.g., N-cyclopropylamino), —N(C₁₋₆alkyl)(C₁₋₆alkyl) (e.g., dimethylamino), —SO₂, —C₁₋₆alkyl (e.g., methylsulfonyl), hydroxy(C₁₋₆alkyl) (e.g., 2-hydroxy-2-propyl), —N(C₁₋₆alkyl)(SO₂—C₁₋₆alkyl) (e.g., N-methyl-N-(methylsulfonyl)amino), —N(C₁₋₆alkyl)(CO—C₁₋₆alkyl) (e.g., N-methyl-N-(acetyl)amino), or heterocycloalkyl (e.g., 4-morpholinyl, 1-pyrrolidinyl, 1-azetidinyl, 1,3-dioxolan-2-yl, 4-tetrahydropyranyl), or a combination thereof;
1.76 The compound of Formula 1.75, wherein B is selected from —CH₂— (morpholine), —CH₂— (piperidine), —CH₂— (piperazine), and —CH₂— (pyrrolidine), each optionally substituted, for example, by halo (e.g., F), C₁₋₆alkyl (e.g., methyl, isopropyl), C₃₋₆cycloalkyl (e.g., cyclopropyl, cyclobutyl), —NH(C₁₋₆alkyl) (e.g., N-methylamino, N-methylcyclopropylamino), —NH(C₃₋₆cycloalkyl), (e.g., N-cyclopropylamino), —N(C₁₋₆alkyl)(C₁₋₆alkyl) (e.g., dimethylamino), —SO₂, —C₁₋₆alkyl (e.g., methylsulfonyl), hydroxy(C₁₋₆alkyl) (e.g., 2-hydroxy-2-propyl), —N(C₁₋₆alkyl)(SO₂—C₁₋₆alkyl) (e.g., N-methyl-N-(methylsulfonyl)amino), —N(C₁₋₆alkyl)(CO—C₁₋₆alkyl) (e.g., N-methyl-N-(acetyl)amino), or heterocycloalkyl (e.g., 4-morpholinyl, 1-pyrrolidinyl, 1-azetidinyl, 1,3-dioxolan-2-yl, 4-tetrahydropyranyl), or a combination thereof;
1.77 The compound of Formula 1.75 or 1.76, wherein the —CH₂— of the CH₂—(3- to 6-membered heterocycloalkyl) is substituted by a methyl group, i.e., it is a —CH(CH₃)-(3- to 6-membered heterocycloalkyl) (e.g., CH(CH₃)-(piperidine), CH(CH₃)-(piperazine), or CH(CH₃)-(pyrrolidine), each optionally further substituted as set forth above;
1.78 The compound of Formula I, or any of 1.1-1.63, wherein B is optionally substituted —(CO)-(3- to 6-membered heterocycloalkyl);
1.79 The compound of Formula 1.78, wherein B is —(CO)-(3- to 6-membered heterocycloalkyl) and said heterocycloalkyl is selected from morpholine, piperidine, piperazine, tetrahydropyran, pyrrolidine, tetrahydrofuran, oxetane, azetidine, oxirane, and aziridine, each optionally substituted, for example, by halo (e.g., F), C₁₋₆alkyl (e.g., methyl, isopropyl), C₃₋₆cycloalkyl (e.g., cyclopropyl, cyclobutyl), —NH(C₁₋₆alkyl) (e.g., N-methylamino, N-methylcyclopropylamino), —NH(C₃₋₆cycloalkyl), (e.g., N-cyclopropylamino), —N(C₁₋₆alkyl)(C₁₋₆alkyl) (e.g., dimethylamino), —SO₂, —C₁₋₆alkyl (e.g., methylsulfonyl), hydroxy(C₁₋₆alkyl) (e.g., 2-hydroxy-2-propyl), —N(C₁₋₆alkyl)(SO₂—C₁₋₆alkyl) (e.g., N-methyl-N-(methylsulfonyl)amino), —N(C₁₋₆alkyl)(CO—C₁₋₆alkyl) (e.g., N-methyl-N-(acetyl)amino), or heterocycloalkyl (e.g., 4-morpholinyl, 1-pyrrolidinyl, 1-azetidinyl, 1,3-dioxolan-2-yl, 4-tetrahydropyranyl), or a combination thereof;
1.80 The compound of Formula 1.79, wherein B is selected from —(CO)-(morpholine), —(CO)-(piperidine), —(CO)-(piperazine), and —(CO)-(pyrrolidine), each optionally substituted, for example, by halo (e.g., F), C₁₋₆alkyl (e.g., methyl, isopropyl), C₃₋₆cycloalkyl (e.g., cyclopropyl, cyclobutyl), —NH(C₁₋₆alkyl) (e.g., N-methylamino, N-methylcyclopropylamino), —NH(C₃₋₆cycloalkyl), (e.g., N-cyclopropylamino), —N(C₁₋₆alkyl)(C₁₋₆alkyl) (e.g., dimethylamino), —SO₂, —C₁₋₆alkyl (e.g., methylsulfonyl), hydroxy(C₁₋₆alkyl) (e.g., 2-hydroxy-2-propyl), —N(C₁₋₆alkyl)(SO₂—C₁₋₆alkyl) (e.g., N-methyl-N-(methylsulfonyl)amino), —N(C₁₋₆alkyl)(CO—C₁₋₆alkyl) (e.g., N-methyl-N-(acetyl)amino), or heterocycloalkyl (e.g., 4-morpholinyl, 1-pyrrolidinyl, 1-azetidinyl, 1,3-dioxolan-2-yl, 4-tetrahydropyranyl), or a combination thereof;
1.81 The compound of Formula I, or any of 1.1-1.63, wherein B is optionally substituted C₁₋₆alkyl (e.g., wherein B is methyl, ethyl, propyl, isopropyl n-butyl, s-butyl, or t-butyl, optionally further substituted by methyl, ethyl, etc.), for example, substituted by OH, or C₁₋₆alkoxy (e.g., methoxy);
1.82 The compound of Formula I, or any of 1.1-1.63, wherein B is optionally substituted C₃₋₆cycloalkyl (e.g., wherein B is cyclopropyl, or cyclobutyl), for example, substituted by OH, or C₁₋₆alkoxy (e.g., methoxy);
1.83 The compound of Formula I, or any of 1.1-1.63, wherein B is optionally substituted —CH₂—(6- to 12-membered bicyclic heterocycloalkyl);
1.84 The compound of Formula I, or any of 1.1-1.63, wherein B is optionally substituted —(CO)-(6- to 12-membered bicyclic heterocycloalkyl);
1.85 The compound of Formula 1.83 or 1.84, wherein said bicyclic heterocycloalkyl is a bridged heterocycloalkyl, for example, selected from:

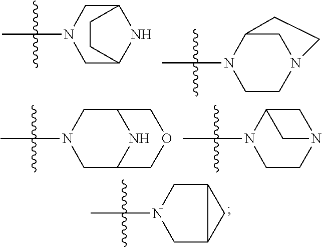

1.86 The compound of formula 1.82 or 1.84, wherein said bicyclic heterocycloalkyl is a spiro heterocycloalkyl, for example, selected from:

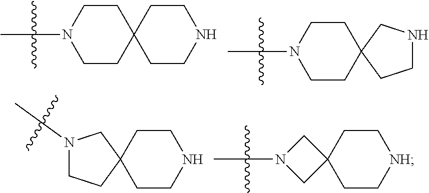 [SMILES: *N1CCC2(CCNCC2)CC1 ; *N1CCC2(CCNC2)CC1 ; *N1CCC2(CCNCC2)C1 ; *N1CC2(CCNCC2)C1]

1.87 The compound of Formula 1.83 or 1.84, wherein said bicyclic heterocycloalkyl is a fused heterocycloalkyl, for example, selected from:

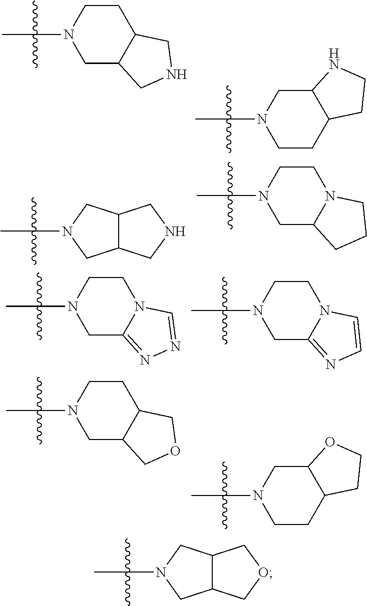

1.88 The compound of Formula 1.85, 1.86, or 1.87, wherein the ring —NH— is substituted by a C₁₋₆alkyl (e.g., methyl) and/or wherein a ring carbon is substituted by a —N(C₁₋₆alkyl)(C₁₋₆alkyl) (e.g., —NMe₂);
1.89 The compound of Formula I, or any of Compounds 1.1-1.88, wherein substituent B is substituted with one or more groups selected from OH, CN, C₁₋₆alkyl (e.g., methyl, isopropyl, 2-hydroxy-2-propyl), halogen (e.g., F), C₁₋₆alkoxy (e.g., methoxy), haloC₁₋₆alkyl (e.g., CF₃), carboxy (COOH), —NH(C₁₋₆alkyl) (e.g., N-methylamino, N-methylcyclopropylamino), —NH(C₃₋₆cycloalkyl) (e.g., N-cyclopropylamino), —N(C₁₋₆alkyl)(C₁₋₆alkyl) (e.g., dimethylamino), —SO—C₁₋₆alkyl(e.g., methylsulfonyl), —SO₂—C₁₋₆alkyl, —NH—SO₂—(C₁₋₆alkyl), —N(C₁₋₆alkyl)-SO₂—(C₁₋₆alkyl) (e.g., N-methyl-N-(methylsulfonyl)amino), —NH—C(O)—(C₁₋₆alkyl) (e.g., N-acetylamino), —N(C₁₋₆alkyl)-C(O)—(C₁₋₆alkyl) (e.g., N-methyl-N-(acetyl)amino), aryl, heteroaryl, C₃₋₆cycloalkyl (e.g., cyclopropyl, cyclobutyl), and 3- to 6-membered heterocycloalkyl (e.g., 4-morpholinyl, 1-pyrrolidinyl, 1-azetidinyl, 1,3-dioxolan-2-yl, 4-tetrahydropyranyl), or a combination thereof, wherein said alkyl, alkoxy, aryl, heteroaryl, cycloalkyl, and heterocycloalkyl is each optionally independently substituted with one or more groups selected from OH, CN, C₁₋₆alkyl (e.g., methyl or t-butyl), halogen (e.g., F or Br), C₁₋₆alkoxy (e.g., methoxy), haloC₁₋₆alkyl (e.g., CF₃), carboxy (COOH), C₃₋₆cycloalkyl, and 5- or 6-membered heterocycloalkyl;
1.90 The compound of Formula 1.89, wherein substituent B is substituted with one or two groups selected from halogen (e.g., F), C₁₋₆alkyl (e.g., methyl, isopropyl, 2-hydroxy-2-propyl), C₃₋₆cycloalkyl (e.g., cyclopropyl, cyclobutyl), —NH(C₁₋₆alkyl) (e.g., N-methylamino, N-methylcyclopropylamino), —NH(C₃₋₆cycloalkyl) (e.g., N-cyclopropylamino), —N(C₁₋₆alkyl)(C₁₋₆alkyl) (e.g., dimethylamino), —SO—C₁₋₆alkyl (e.g., methylsulfonyl), —SO₂—C₁₋₆alkyl, —NH—SO₂—(C₁₋₆alkyl), —N(C₁₋₆alkyl)-SO₂—(C₁₋₆alkyl) (e.g., N-methyl-N-(methylsulfonyl)amino), —NH—C(O)—(C₁₋₆alkyl), —N(C₁₋₆alkyl)-C(O)—(C₁₋₆alkyl) (e.g., N-methyl-N-acetylamino), and 3- to 6-membered heterocycloalkyl (e.g., 4-morpholinyl, 1-pyrrolidinyl, 1-azetidinyl, 1,3-dioxolan-2-yl, 4-tetrahydropyranyl), wherein said alkyl is optionally independently substituted with one or more groups selected from OH and halogen;
1.91 The compound of Formula 1.90, wherein each of said C₁₋₆alkyl is methyl or ethyl;
1.92 The compound of Formula I, or any one of Formulas 1.1-1.91, wherein group B is —CH₂— (piperidine), —CH₂— (piperazine), —CH₂— (pyrrolidine), —CH₂— (azetidine), —(CO)-(piperidine), —(CO)-(piperazine), or —(CO)-(pyrrolidine), substituted by one or two groups selected from the group consisting of fluoro, C₁₋₆alkyl (e.g., methyl), —N(C₁₋₆alkyl)(C₁₋₆alkyl) (e.g., —NMe₂), —SO₂—C₁₋₆alkyl (e.g., —SO₂-Me), —N(C₁₋₆alkyl)-SO₂—(C₁₋₆alkyl) (e.g., —N(Me)-SO₂-Me), —N(C₁₋₆alkyl)-C(O)—(C₁₋₆alkyl) (e.g., —N(Me)-C(O)-Me), azetidinyl (e.g., 1-azetidinyl), pyrrolidinyl (e.g., 1-pyrrolidinyl), and C₁₋₆alkyl substituted by OH (e.g., —C(Me)₂OH), or wherein group B is unsubstituted —CH₂— (morpholine);
1.93 The compound of Formula 1.92, wherein said group B is substituted by a single one of said groups;
1.94 The compound of Formula 1.92, said group B is substituted by one fluoro and one group selected from —N(C₁₋₆alkyl)(C₁₋₆alkyl) (e.g., —NMe₂), azetidinyl (e.g., 1-azetidinyl), and pyrrolidinyl (e.g., 1-pyrrolidinyl);
1.95 The compound of any of Formulas 1.92-1.94, wherein the —CH₂— of the CH₂—(3- to 6-membered heterocycloalkyl) is substituted by a methyl group, i.e., it is a —CH(CH₃)-(piperidine), CH(CH₃)-(piperazine), or CH(CH₃)-(pyrrolidine), each optionally further substituted as set forth above;
1.96 The compound of Formula I, or any one of Formulas 1.1-1.91, wherein group B is —(CO)—N(Rₐ)—R₂, optionally wherein Rₐ is H or methyl, and R₂ is optionally substituted C₁₋₆alkyl, optionally substituted C₃₋₆cycloalkyl, or optionally substituted 3- to 7-membered heterocycloalkyl;
1.97 Compound 1.96, wherein Rₐ is H and R₂ is tetrahydropyranyl, oxetanyl, cyclopropyl, 1-(cyclopropyl)ethyl, piperidinyl, 1-methyl-4-piperidinyl, 1-methyl-3-piperidinyl, pyridyl, 1-methyl-3-pyrrolidinyl, or 1-methyl-3-azetidinyl;
1.98 The compound of Formula I, or any one of Formulas 1.1-1.91, wherein group B is N(Rₐ)—R₂, optionally wherein Rₐ is H or methyl, and R₂ is optionally substituted C₁₋₆alkyl, optionally substituted C₃₋₆cycloalkyl, or optionally substituted 3- to 7-membered heterocycloalkyl;
1.99 Compound 1.98, wherein Rₐ is H or methyl, and R₂ is 1-methyl-4-piperidinyl or 2-(methylsulfonyl)ethyl;
1.100 The compound of Formula I, or any one of Formulas 1.1-1.91, wherein B is N(Rₐ)—(CO)—R₂, optionally wherein Rₐ is H or methyl, and R₂ is optionally substituted C₁₋₆alkyl, optionally substituted C₃₋₆cycloalkyl, or optionally substituted 3- to 7-membered heterocycloalkyl;
1.101 Compound 1.100, wherein Rₐ is H and R₂ is tetrahydropyranyl or morpholinyl;
1.102 The compound of Formula I, or any of 1.1-1.101, wherein group B is selected from the group consisting of:

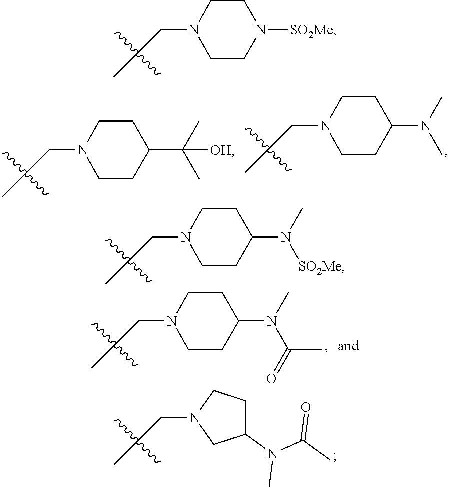

1.103 The compound of Formula I, or any of 1.1-1.101, wherein group B is selected from the group consisting of:

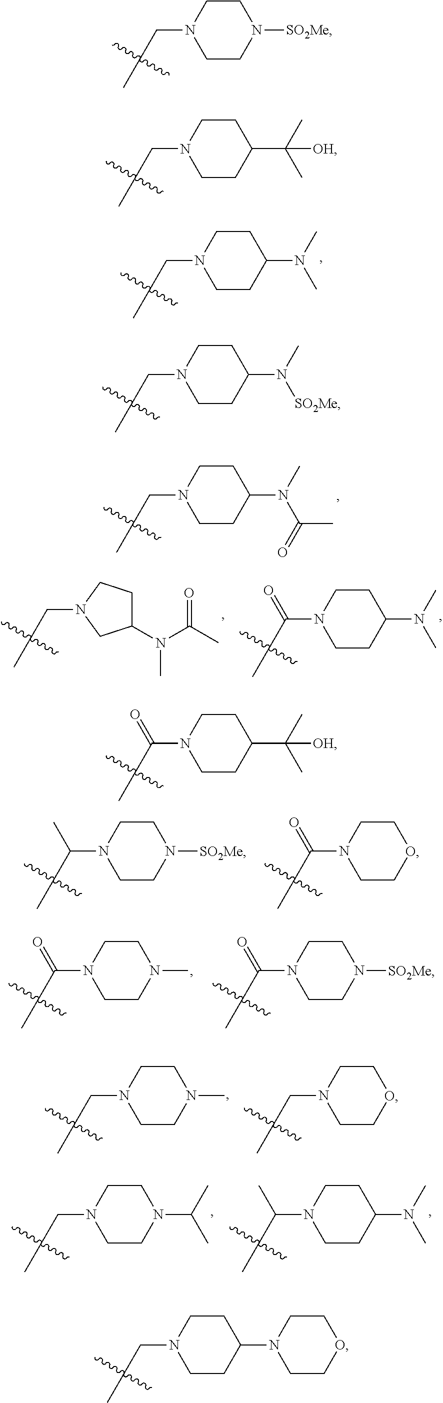

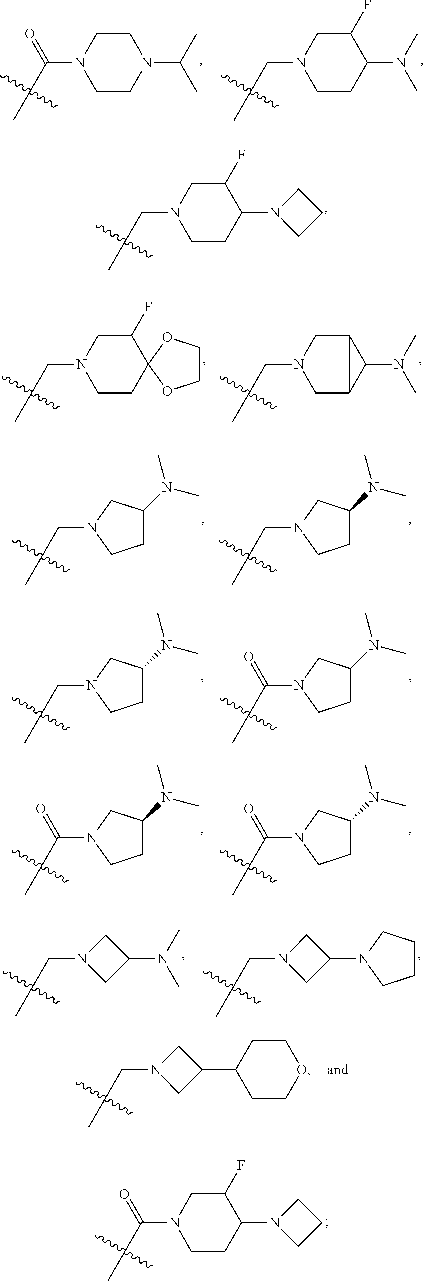

1.104 The compound of Formula I, or any of 1.1-1.101, wherein group B is selected from the group consisting of:

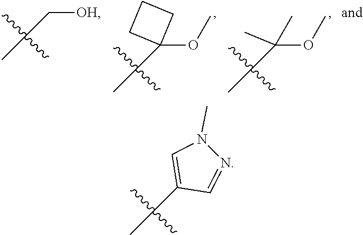

1.105 The compound of Formula I, or any of 1.1-1.101, wherein group B is selected from the group consisting of:

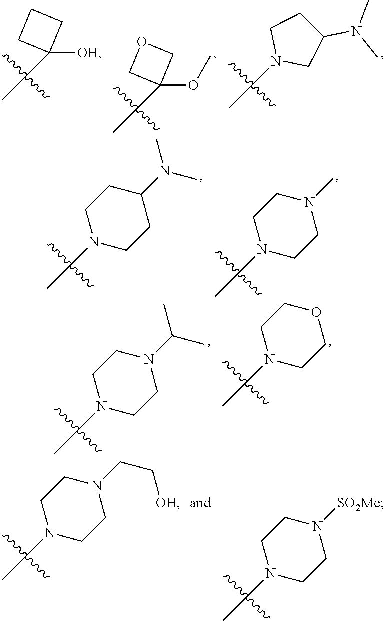

1.106 The compound of Formula I, or any of 1.1-1.101, wherein group B is selected from the group consisting of:

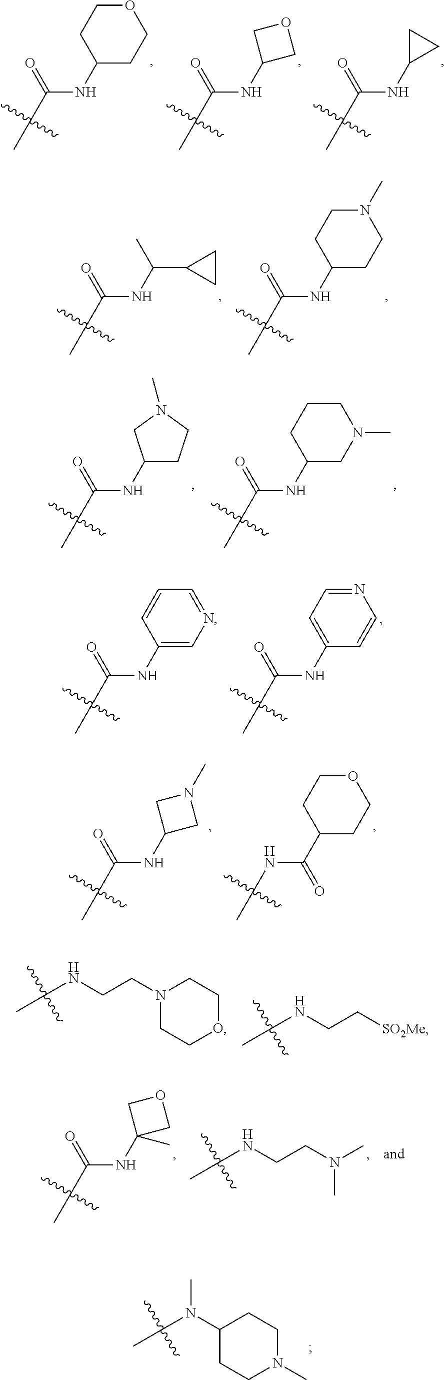

1.107 The compound of Formula I, or any of 1.1-1.101, wherein group B is selected from the group consisting of:

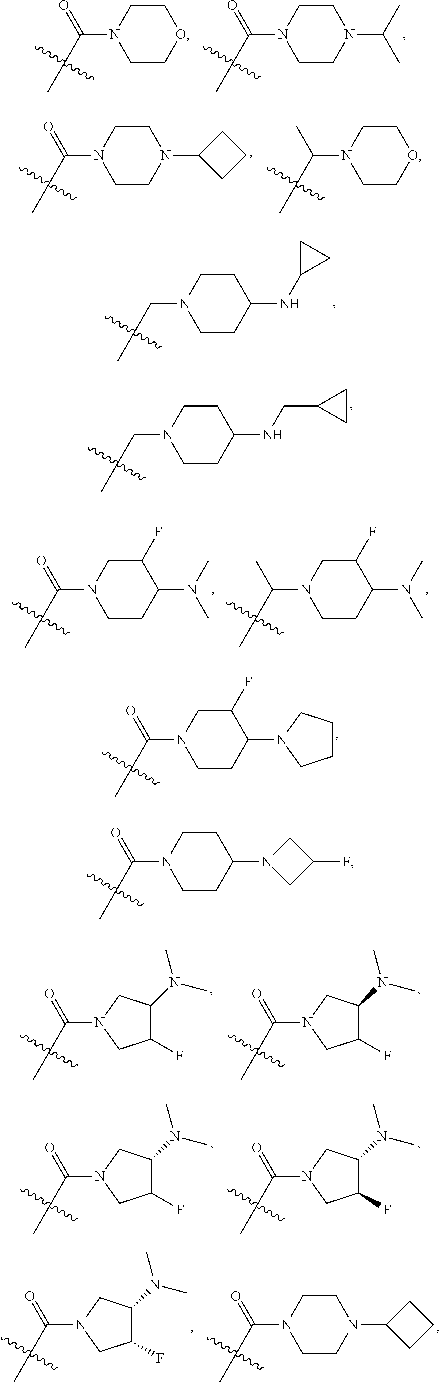

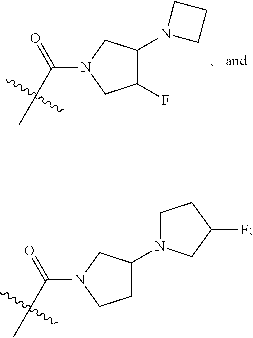 [SMILES: *C(=O)N1CC(F)C(N2CCC2)C1 ; *C(=O)N1CCC(N2CCC(F)C2)C1]

1.108 The compound of Formula I, or any of 1.1-1.101, wherein group B is selected from the group consisting of:

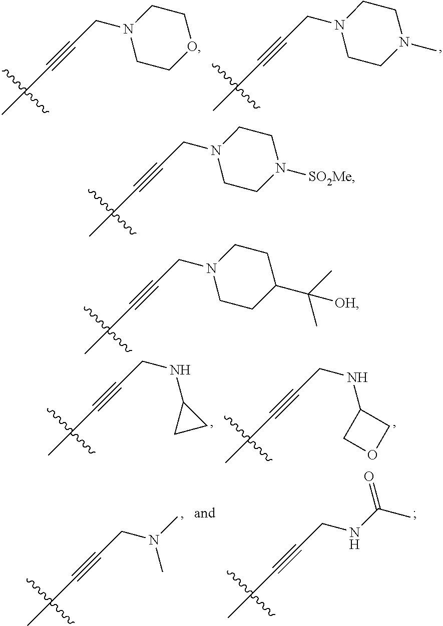

1.109 The compound of Formula I, or any of 1.1-1.101, wherein group B is selected from the group consisting of:

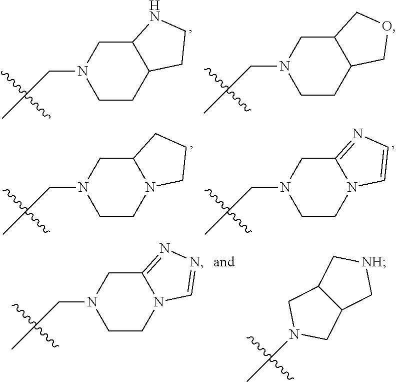

1.110 The compound of Formula I, or any of 1.1-1.101, wherein group B is selected from the group consisting of:

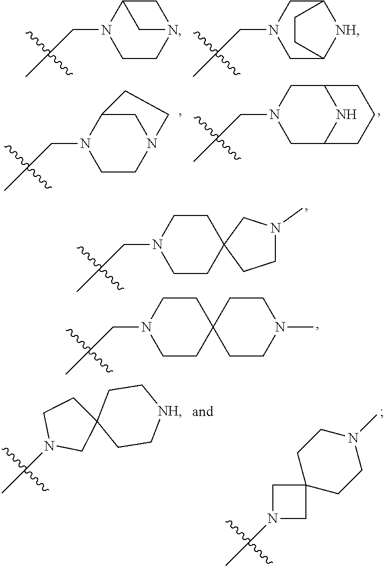

1.111 The compound of Formula I, or any of 1.1-1.101, wherein group B is selected from the group consisting of:

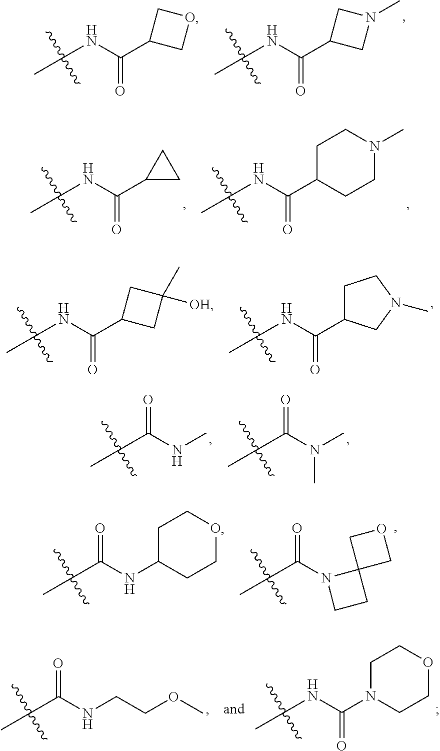

1.112 The compound of Formula I, or any of Compounds 1.1-1.111, wherein substituent B is unsubstituted;
1.113 The compound of Formula I, or any of Compounds 1.1-1.112, wherein R₁ is C₁₋₆alkyl, C₃₋₆cycloalkyl, or C₁₋₆alkoxy, each substituted with an optionally substituted 3- to 7-membered heterocycloalkyl;
1.114 The compound of Formula I, or any of Compounds 1.1-1.112, wherein R₁ is —C(O)—R₂, —C(O)O—R₂, —OC(O)—R₂, —C(O)N(Rₐ)—R₂, —N(Rₐ)C(O)—R₂, —N(Rₐ)—R₂, or —O—R₂; and wherein R₂ is an optionally substituted 3- to 7-membered heterocycloalkyl;
1.115 The compound of Formula I, or any of Compounds 1.1-1.112, wherein R₁ is an optionally substituted 3- to 7-membered heterocycloalkyl;
1.116 The compound of any of Formulas 1.113-1.115, wherein said heterocycloalkyl is selected from aziridine, azetidine, oxirane, oxetane, pyrrolidine (e.g., 3,3-difluoropyrrolidin-1-yl), pyrrolidinone (e.g., 1-pyrrolidin-3-one), tetrahydrofuran, tetrahydropyran (e.g., tetrahydropyran-4-yl), dihydropyran (e.g., 3,6-dihydropyran-4-yl), morpholine, piperidine, piperazine, and oxa-azaspiro[3.3]heptane (e.g., 2-oxa-6-azaspiro[3.3]heptan-6-yl), 1,4-oxazepan-4-yl, 3-oxa-6-azabicyclo[3.1.1]heptan-6-yl, and 2-oxa-5-azabicyclo[2.2.1]heptan-5-yl, wherein each said heterocycloalkyl is optionally substituted;
1.117 The compound of Formula 1.116, wherein said heterocycloalkyl is optionally substituted morpholine (e.g., 2-methylmorpholin-4-yl, or 3-methylmorpholin-4-yl, or morpholin-4-yl (i.e., N-morpholinyl));
1.118 The compound of Formula 1.116, wherein said heterocycloalkyl is selected from (e.g., 3,3-difluoropyrrolidin-1-yl), 1-pyrrolidin-3-one, and 2-oxa-6-azaspiro[3.3]heptan-6-yl;
1.119 The compound of any of Formulas 1.113-1.118, wherein said heterocycloalkyl is substituted with one or more groups selected from OH, CN, C₁₋₆alkyl (e.g., methyl), halogen (e.g., F), C₁₋₆alkoxy (e.g., methoxy), haloC₁₋₆alkyl (e.g., CF₃), and carboxy (COOH);
1.120 The compound of any of Formulas 1.113-1.118, wherein said heterocycloalkyl is unsubstituted (e.g., unsubstituted morpholin-4-yl);
1.121 The compound according to Formula I or any of Formulas 1.1-1.120, wherein the compound is selected from:

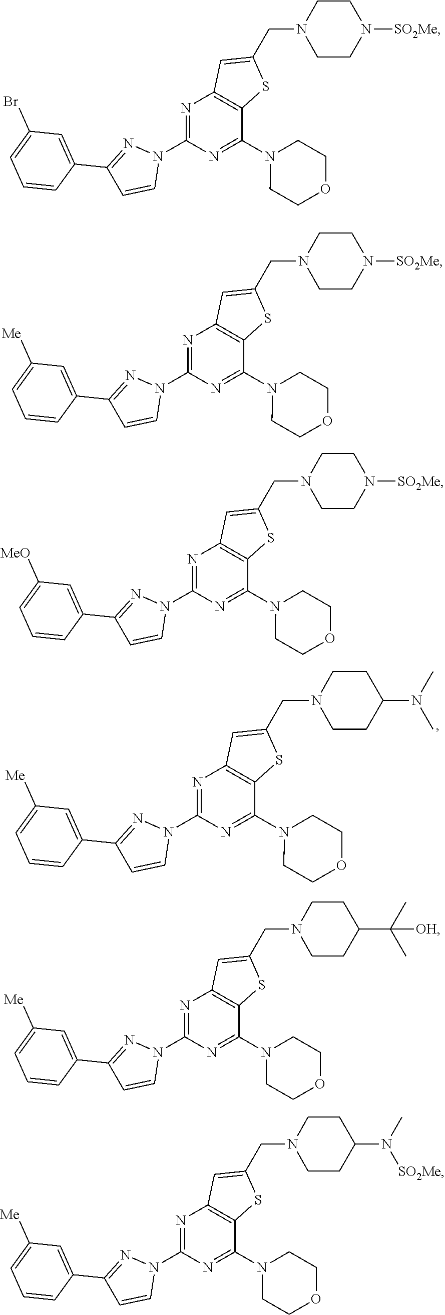

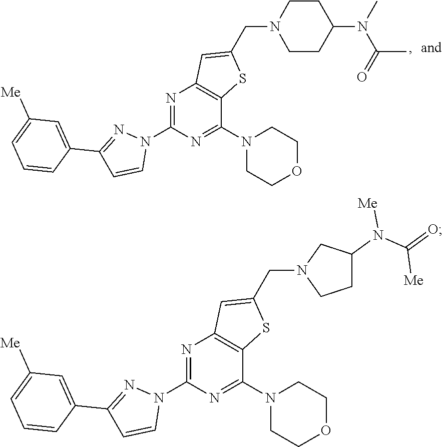 [SMILES: CC(=O)N(C)C1CCN(Cc2cc3nc(-n4ccc(-c5cccc(C)c5)n4)nc(N4CCOCC4)c3s2)CC1 ; CC(=O)N(C)C1CCN(Cc2cc3nc(-n4ccc(-c5cccc(C)c5)n4)nc(N4CCOCC4)c3s2)C1]

1.122 The compound according to Formula I or any of Formulas 1.1-1.120, wherein the compound is selected from the compounds listed in Table 1 below;
1.123 The compound according to Formula I or any of Formulas 1.1-1.120, wherein the compound is selected from the compounds listed in Table 2 below;
1.124 The compound according to Formula I or any of Formulas 1.1-1.123, wherein the compound is in free base form;
1.125 The compound according to Formula I or any of Formulas 1.1-1.123, wherein the compound is in the form of a pharmaceutically acceptable acid addition salt (e.g., hydrochloride);
1.126 The compound according to Formula I or any of Formulas 1.1-1.125, wherein the compound is an inhibitor of PIKFYVE (e.g., the compound having a Ki or IC₅₀ of less than 10 μM, or less than 1 μM, or less than 100 nM, or less than 50 nM, or less than 25 nM, or less than 10 nM; and/or the compound provides >50% inhibition at a concentration of 1 μM, or >75%, or >85% or >90% inhibition at said concentration;
   in free or pharmaceutically acceptable salt form.

In some embodiments, it is further provided that the compound of Formula I as hereinbefore defined does not include in its scope any compound, in free or any salt form, specifically disclosed in any of the following references: WO 2007/127183, WO 2009/013545, WO 2009/042607, WO 2009/053716, WO 2010/138589, WO 2012/104776, WO 2016/157074, WO 2019/113523, WO2021/057256 (Chinese), WO 2023/055181 (Chinese), U.S. Pat. Nos. 7,750,556, and 8,044,068.

In a second aspect, the invention provides a pharmaceutical composition comprising the compound of Formula I or any of 1.1-1.126 as described herein, in free or pharmaceutically acceptable salt form, in admixture with a pharmaceutically acceptable diluent or carrier. In some embodiments, the composition is a composition for oral administration, such as a tablet or capsule. In some embodiments, such an oral dosage form is an immediate-release composition, or a delayed release composition, or a sustained release composition. In other embodiments, the pharmaceutical composition is an injectable composition, such as for intravenous, intramuscular, intrathecal, intraabdominal, intraperitoneal, or subcutaneous injection. In other embodiments, the pharmaceutical composition may be an inhalational composition, including powdered and aerosol compositions (i.e., gas liquid/emulsions), such as an intranasal composition (e.g., spray) or an intrapulmonary composition (e.g., metered dose inhaler).

Pharmaceutical compositions include all compositions wherein the compounds of the present invention are contained in an amount that is effective to achieve its intended purpose. While individual needs vary, determination of optimal ranges of effective amounts of each component is within the skill of the art. Typically, the compounds may be administered to mammals, e.g., humans, orally at a dose of 0.0025 to 50 mg/kg, or an equivalent amount of the pharmaceutically acceptable salt thereof, per day of the body weight of the mammal being treated for insomnia. For intramuscular injection, the dose is generally about one-half of the oral dose.

The unit oral dose may comprise from about 0.01 to about 1000 mg, preferably about 0.1 to about 100 mg of the compound, or 0.1 to 50 mg. The unit dose may be administered one or more times daily as needed to achieve the desired intended daily dosage.

The compounds of Formula I or any of 1.1-1.126 as described herein are highly effective inhibitors of PIKFYVE, preferably producing inhibition at nanomolar concentrations. In some embodiments the compounds are selective PIKFYVE inhibitors, e.g., the compounds have little or no inhibitory activity of other kinases, for example, other lipid kinases (e.g., other phosphoinositide kinases, such as phosphoinositide 3-kinases, phosphoinositide 4-kinases, phosphoinositide 5-kinases, phosphoinositide-5-phosphate 4-kinases, and phosphatidyl inositol 4-phosphate 5-kinases), and protein kinases (e.g., tyrosine kinases and serine-threonine kinases). In some embodiments, the compounds have a Kd or IC₅₀ of greater than 100 nM, or greater than 500 nM, or greater than 1000 nM, or greater than 10,000 nM, or greater than 50,000 nM against one or more of these other kinases, and/or the compound provides <50% inhibition at a concentration of 1 μM, or <25%, or <10%% or <5%, or <1% inhibition at said concentration against one or more of these other kinases.

PIKFYVE inhibitors according to the invention are therefore useful for treatment and prophylaxis of diseases and disorders which may be ameliorated by modulating (e.g., inhibiting) PIKFYVE-dependent signaling pathways or by modulating (e.g., inhibiting) endosome formation or trafficking.

Therefore, in the third aspect, the invention provides a method for the treatment or prophylaxis of a disease or disorder characterized by dysregulation of phosphoinositide-mediated signal transduction pathways or which may be ameliorated by modulating (e.g., inhibiting) PIKFYVE-dependent signaling pathways or by modulating (e.g., inhibiting) endosome formation or trafficking, comprising administering to a patient in need thereof an effective amount of the compound of Formula I, or any of formulae 1.1-1.126 as described herein, in free or pharmaceutically acceptable salt form.

In some embodiments, the disease or disorder is a hyperproliferative disease (e.g., cancer, such as MET- or RAS-dependent cancers), an autoimmune disease (such as Crohn's disease or rheumatoid arthritis), a neurological disease (such as amyotrophic lateral sclerosis (ALS) and fronto-temporal dementia (FTD), and in particular C9FTD/ALS), diabetes or prediabetes, or Francois-Neetens corneal fleck dystrophy. In some embodiments, the disease or disorder is a cancer, such as a cancer having a genotype or phenotype indicative of PIKFYVE overactivity or Sac underactivity. Cancer which may be amenable to treatment with a PIKFYVE inhibitor include, but are not limited to, non-Hodgkin lymphoma, multiple myeloma, melanoma, liver cancer, glioblastoma, multiple myeloma, prostate cancer and breast cancer. In particular embodiments, the cancer is castration-resistant prostate cancer. In particular embodiments, the cancer having activated MET or RAS signaling pathways. In some embodiments the disease or disorder is infection by an enveloped virus, such as a virus which gains cellular entry by endocytosis. Such viruses include, but are not limited to, Ebola, influenza A, vesicular stomatitis virus, Lassa fever virus, lymphocytic choriomeningitis virus, and coronaviruses (including MERS-CoV, SARS-CoV and SARS-CoV-2). Thus, in another embodiment, the present disclosure provides a method for treating or preventing a viral infection by an enveloped virus, such as Ebola, influenza A, vesicular stomatitis virus, Lassa fever virus, lymphocytic choriomeningitis virus, and coronaviruses (including MERS-CoV, SARS-CoV and SARS-CoV-2).

The PIKFYVE inhibitor compounds described herein for the treatment or prophylaxis of a disease or disorder according to the foregoing methods may be used as a sole therapeutic agent or may be used in combination with one or more other therapeutic agents useful for the treatment of said diseases or disorders.

Such other agents include inhibitors of other protein kinases or other proteins associated with cancer development (for example, serine-threonine kinases, tyrosine kinases, growth factor receptors), traditional cytotoxic anticancer agents (e.g., DNA alkylating agents, antimetabolites, anti-microtubule agents, topoisomerase inhibitors, and cytotoxic antibiotics), and monoclonal antibody therapies (e.g., pembrolizumab, rituximab, trastuzumab, alemtuzumab, cetuximab, panitumumab, bevacizumab, and ipilimumab).

Small molecule targeted therapies include inhibitors of such proteins as Bcr-Abl kinase, PDGFR, EGFR, VEGFR, RAF kinases, Ras-kinases, c-Kit, Src kinase, ephrin receptors, HER2/neu (ErbB2), proteasomes, estrogen receptors, JAK kinase, ALK, Bcl-2, PARP, PI3K, Braf, MEK, MAPK, CDK, HSP90, mTOR, inhibitors of checkpoint proteins (e.g., PD1, PDL1 and CTLA inhibitors), and modulators of the adaptive and innate immune system. Examples of these small molecule inhibitors include imatinib, gefitinib, erlotinib, sorafenib, sunitinib, dasatinib, lapatinib, nilotinib, bortezomib, tamoxifen, tofacitinib, crizotinib, obatoclax, navitoclax, gossypol, iniparib, olaparib, perifosine, apatinib, vemurafenib, dabrafenib, trametinib, CDK inhibitors, temsirolimus, everolimus, vemurafenib, and trametinib. Cytotoxic chemotherapeutic agents include cyclophosphamide, chlormethine, uramustine, melphalan, chlorambucil, ifosfamide, bendamustine, carmustine, lomustine, streptozotocin, busulfan, cisplatin, carboplatin, dicycloplatin, eptaplatin, lobaplatin, miriplatin, nedaplatin, oxaliplatin, picoplatin, satraplatin, triplatin, procarbazine, altretamine, dacarbazine, temozolomide, 5-fluorouracil, 6-mercaptopurine, thioguanine, capecitabine, azacytidine, decitabine, nelarabine, cladribine, clofarabine, cytarabine, floxuridine, fludarabine, gemcitabine, pentostatin, hydroxycarbamide, methotrexate, pemetrexed, daunorubicin, doxorubicin, epirubicin, idarubicin, actinomycin-D, bleomycin, mitomycin-C, mitoxantrone, vincristine, vinblastine, vinorelbine, vindesine, vinflunine, paclitaxel, docetaxel, etoposide, teniposide, irinotecan, topotecan, novobiocin, aclarubicin, pirarubicin, and aclarubicin. In some embodiments, particularly for the treatment of a cancer having activated MET or RAS signaling pathways, the compounds of the present disclosure are combined with compounds which inhibit MET activity or inhibit RAS activity, or inhibit upstream or downstream effectors in the MET or RAS signaling pathways, such as salirasib, tipifarnib, lonafarnib, crizotinib, cabozanitib, tivantinib, and tepotinib. In a particular embodiment, the cancer to be treated is a prostate cancer (e.g., a castration-resistant prostate cancer and the compound of the present disclosure is combined with an anti-PD-1 antibody or a PD-1 inhibitor, such as pembrolizumab.

Such other agents also include small-molecule antiviral agents, such entry inhibitors, uncoating inhibitors, transcription or reverse transcription inhibitors, integrase inhibitors, translation inhibitors, protease inhibitors, assembly inhibitors, release inhibitors, and immune system stimulants (e.g., interferons). Examples of such agents include: abacavir, acyclovir, adefovir, amantadine, ampligen, amprenavir, arbidol umfenovir, atazanavir, atripla, baloxavir marboxil, biktarvy, boceprevir, bulevirtide, cidofovir, cobicistat, combivir, daclatasvir, darunavir, delavirdine, descovy, didanosine, docosanol, dolutegravir, doravirine, edoxudine, efavirenz, elvitegravir, emtricitabine, enfuvirtide, entecavir, etravirine, famciclovir, favipiravir, fomivirsen, fosamprenavir, foscarnet, ganciclovir, ibacitabine, ibalizumab, idoxuridine, imiquimod, imunovir, indinavir, lamivudine, letermovir, lopinavir, lopinavir/ritonavir combination, loviride, maraviroc, methisazone, molnupiravir, moroxydine, nelfinavir, nevirapine, nexavir, nirmatrelvir, nirmatrelvir/ritonavir combination, nitazoxanide, norvir, oseltamivir, penciclovir, peramivir, pleconaril, raltegravir, remdesivir, ribavirin, rifampicin, rimantadine, ritonavir, saquinavir, simeprevir, sofosbuvir, stavudine, taribavirin, telaprevir, telbivudine, tenofovir, tenofovir alafenamide, tenofovir disoproxil, tipranavir, trifluridine, trizivir, tromantadine, Truvada (emtricitabine/tenofovir combination), umfenovir, valaciclovir, valganciclovir, vicriviroc, vidarabine, zalcitabine, zanamivir, and zidovudine. In a preferred embodiment, especially when the treatment is directed to a coronavirus infection, the other agents may be selected from lopinavir, molnupiravir, nirmatrelvir, remdesivir, ritonavir, tenofovir, lopinavir/ritonavir combination, and nirmatrelvir/ritonavir combination.

Other agents that might be combined with the compounds of the present invention include: corticosteroids, methotrexate, thiopurine, chloroquine, hydroxychloroquine, sulfasalazine, leflunomide, certolizumab, infliximab, etanercept, abatacept, anakinra, rituximab, tocilizumab, cyclosporin, golimumab, adalimumab, insulin, exenatide, liraglutide, pramlintide, metformin, phenformin, buformin, rosiglitazone, pioglitazone, troglitazone, tolbutamide, acetohexamide, tolazamide, chlorpropamide, glipizide, glyburide, glimepiride, gliclazide, glyclopyramide, gliquidone, meglitinide, repaglinide, nateglinide, miglitol, acarbose, voglibose, taspoglutide, lixisenatide, semaglutide, dulaglutide, vildagliptin, sitagliptin, saxagliptin, linagliptin, alogliptin, septagliptin, teneligliptin, gemigliptin, dapagliflozin, canagliflozin, empagliflozin, and remogliflozin,

In another aspect, the invention provides the following
(i) the compound of Formula I or any of 1.1-1.126 as described herein, in free or pharmaceutically acceptable salt form, for use in any of the methods or in the treatment or prophylaxis of any disease or disorder as set forth herein,
(ii) a combination as described hereinbefore, comprising a compound of Formula I or any of 1.1-1.126 as described herein, in free or pharmaceutically acceptable salt form and a second therapeutic agent useful for the treatment or prophylaxis of any disease or disorder set forth herein;
(iii) use of the compound of Formula I or any of 1.1-1.126, in free or pharmaceutically acceptable salt form, or the combination described herein, (in the manufacture of a medicament) for the treatment or prophylaxis of any disease or condition as set forth herein,
(iv) the compound of Formula I or any of 1.1-1.126, in free or pharmaceutically acceptable salt form, the combination described herein or the pharmaceutical composition of the invention as hereinbefore described for use in the treatment or prophylaxis of any disease or condition as set forth herein.

If not otherwise specified or clear from context, the following terms herein have the following meanings
(a) “Alkyl” as used herein is a saturated or unsaturated hydrocarbon moiety, preferably saturated, preferably having one to six carbon atoms, in some embodiments, one to four carbon atoms, which may be linear or branched, and may be optionally mono-, di- or tri-substituted, e.g., with halogen (e.g., chloro or fluoro) or hydroxy. Exemplary “C₁₋₆ alkyl” groups include methyl, ethyl, n-propyl, isopropyl, n-butyl, isobutyl, tert-butyl, etc. Of course, other “C₁₋₆ alkyl” groups will be readily apparent to those of skill in the art given the benefit of the present disclosure.
(b) “Aryl” as used herein means any carbocyclic aromatic ring system, i.e., any aromatic ring system comprising only carbon atoms as ring atoms. This includes 6-membered monocyclic aryl ring systems and 9-membered or 10-membered fused bicyclic aryl ring systems, and larger fused ring systems, as long such ring systems comprise at least one 6-membered aromatic carbocyclic ring (i.e., a benzene ring) within the fused ring system, and as long as no ring-atoms are heteroatoms. Aryl includes phenyl and napthyl.
(c) “Heteroaryl” as used herein means any cyclic heteroaromatic ring system, i.e., any aromatic ring system comprising at least one heteroatom (e.g., N, S, or O) ring atom. This includes 5-membered and 6-membered monocyclic heteroaryl ring systems and 9-membered or 10-membered fused bicyclic heteroaryl ring systems, and larger fused ring systems, as long such ring systems comprise at least one aromatic carbocyclic or aromatic heterocyclic ring within the fused ring system and at least one heteroatom (e.g., N, S or O) ring-atom within the fused ring system (either in an aromatic ring or non-aromatic ring). Heteroaryl therefore includes bicyclic fused ring system selected from aromatic-heteroaromatic, aromatic-heterocyclic, heteroaromatic-carbocyclic, heterocyclic-aromatic, and heteroaromatic-heteroaromatic, as well as larger fused ring systems comprising some combination of benzene, cycloalkane, heterocycloalkane and heteroaromatic rings. Exemplary heteroaryl groups include furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidinyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyridinyl, benzo[b]thiophenyl, 5,6,7,8-tetrahydro-quinolin-3-yl, benzoxazolyl, benzothiazolyl, benzisothiazolyl, benzisoxazolyl, benzimidazolyl, thianapthenyl, isothianapthenyl, benzofuranyl, isobenzofuranyl, isoindolyl, indolyl, indolizinyl, indazolyl, isoquinolyl, quinolyl, phthalazinyl, quinoxalinyl, quinazolinyl and benzoxazinyl, etc. It is understood that for heteroaryl systems in which the both ring carbon atoms and ring heteroatoms have open valencies, bonds can be formed to either such atom types (e.g., C-linked or N-linked). For example, where a pyrazolyl moiety is the group A, substituted at one atom to connect to the core of the compound of Formula I, and substituted at one or more other atoms with other substituent groups, either the core of the Compound of Formula I or any one or more other substituents may be attached to either a pyrazole ring nitrogen atom (N-linked) or a pyrazole ring carbon atom (C-linked).
(d) “Heterocycloalkyl” means any cyclic nonaromatic ring system comprising at least one heteroatom (e.g., N, S, or O) ring atom. This includes 3- to 12-membered monocyclic and fused bicyclic ring systems, and any larger multi-ring fused ring systems, as long such ring systems do not comprise any aromatic carbocyclic or aromatic heterocyclic ring. Exemplary heterocycloalkyl groups include pyrrolidinyl, tetrahydrofuranyl, dihydrofuranyl, tetrahydropyranyl, pyranyl, thiopyranyl, azindinyl, azetidinyl, oxiranyl, methylenedioxyl, chromenyl, barbituryl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, piperizin-2-onyl, piperizin-3-onyl, chromanyl, 2-pyrrolinyl, 3-pyrrolinyl, imidazolidinyl, 2-imidazolidinyl, 1,4-dioxanyl, 8-azabicyclo[3.2.1]octanyl, 3-azabicyclo[3.2.1]octanyl, 3,8-diazabicyclo[3.2.1]octanyl, 2,5-diazabicyclo[2.2.1]heptanyl, 2,5-diazabicyclo[2.2.2]octanyl, octahydro-2H-pyrido[1,2-a]pyrazinyl, 3-azabicyclo[4.1.0]heptanyl, 3-azabicyclo[3.1.0]hexanyl, 2-azaspiro[4.4]nonanyl, 7-oxa-1-azaspiro[4.4]nonanyl, 7-azabicyclo[2.2.2]heptanyl, octahydro-1H-indolyl, etc. In general, the heterocycloalkyl group typically is attached to the main structure via a carbon atom or a nitrogen atom. Of course, other heterocycloalkyl groups will be readily apparent to those of skill in the art given the benefit of the present disclosure.
(e) “Cycloalkyl” means a nonaromatic saturated or unsaturated free radical forming at least one ring consisting essentially of 3 to 10 carbon atoms and a corresponding number of hydrogen atoms. The term “cycloalkyl” therefore includes cycloalkenyl groups, as further defined below. As such, cycloalkyl groups can be monocyclic or polycyclic. Individual rings of such polycyclic cycloalkyl groups can have different connectivities, e.g., fused, bridged, spiro, etc., in addition to covalent bond substitution. Exemplary cycloalkyl groups include cyclopropyl, cyclobutyl, cyclopentyl, cyclohexyl, norbornanyl, bicyclo[3.2.1]octanyl, octahydro-pentalenyl, spiro[4.5]decanyl, cyclopropyl, adamantyl, substituted with cyclobutyl, cyclobutyl substituted with cyclopentyl, cyclohexyl substituted with cyclopropyl, etc. Of course, other cycloalkyl groups will be readily apparent to those of skill in the art given the benefit of the present disclosure.

It is understood that when describing the substituents attached in various positions to the core structure of Formula I, including substituents attached to substituents, in some cases, the substituent may be referred to using the name of the corresponding chemical compound, especially in the case of rings, whereas in some cases the same substituent may be referred to using the name of the corresponding chemical radical (e.g., having an “-yl” suffix), but these terms are interchangeable. For example, when referring to the substituent B, or a heteroaryl ring attached to a substituent A, the terms “pyridine” and “pyridyl” are equivalent, as are the terms “morpholine” and “morpholinyl.” The skilled artisan will recognize that such terms are used to denote attachment of, for example, pyridine or morpholine ring at the designated position, thus converting said ring to a pyridyl or morpholinyl substituent respectively. Absent an indication otherwise, such attachments may be made at any chemically permissible location of the attached ring.

Compounds of the Invention, e.g., the compound of Formula I or any of formulae 1.1-1.126 as described herein, may exist in free or salt form, e.g., as acid addition salts (e.g., hydrochloride).

In this specification unless otherwise indicated, language such as “Compounds of the Invention” is to be understood as embracing the compounds in any form, for example free or acid addition salt form, or where the compounds contain acidic substituents, in base addition salt form. The Compounds of the Invention are intended for use as pharmaceuticals, therefore pharmaceutically acceptable salts are preferred. Salts which are unsuitable for pharmaceutical uses may be useful, for example, for the isolation or purification of free Compounds of the Invention or their pharmaceutically acceptable salts, are therefore also included.

The Compounds of the Invention include their enantiomers, diastereomers and racemates, as well as their polymorphs, hydrates, solvates and complexes. Some individual compounds within the scope of this invention may contain double bonds. Representations of double bonds in this invention are meant to include both the E and the Z isomer of the double bond. In addition, some compounds within the scope of this invention may contain one or more asymmetric centers. This invention includes the use of any of the optically pure stereoisomers as well as any combination of stereoisomers.

The Compounds of the present disclosure may comprise one or more chiral carbon atoms. The compounds thus exist in individual isomeric, e.g., enantiomeric or diastereomeric form or as mixtures of individual forms, e.g., racemic/diastereomeric mixtures. Any isomer may be present in which the asymmetric center is in the (R)-, (S)-, or (R,S)-configuration. The invention is to be understood as embracing both individual optically active isomers as well as mixtures (e.g., racemic/diastereomeric mixtures) thereof.

Accordingly, where a Compound of the Invention can exist in the form of enantiomers, the Compounds of the Invention may be a racemic mixture or it may be predominantly, e.g., in pure, or substantially pure, enantiomeric form, e.g., greater than 70% enantiomeric excess (“ee”), preferably greater than 80% ee, more preferably greater than 90% ee, most preferably greater than 95% ee. Likewise, Accordingly, where a Compound of the Invention can exist in the form of one or more diastereomers, the Compounds of the Invention may an equal mixture of one or more diastereomers, or it may be predominantly, e.g., in pure, or substantially pure, diastereomeric form, e.g., greater than 70% diastereomeric excess (“de”), preferably greater than 80% de, more preferably greater than 90% de, most preferably greater than 95% de. The purification of said isomers and the separation of said isomeric mixtures may be accomplished by standard techniques known in the art (e.g., column chromatography, preparative TLC, preparative HPLC, simulated moving bed and the like).

It is also intended that the Compounds of the Invention encompass their stable and unstable isotopes. Stable isotopes are nonradioactive isotopes which contain one additional neutron compared to the abundant nuclides of the same species (i.e., element). It is expected that the activity of compounds comprising such isotopes would be retained, and such compound would also have utility for measuring pharmacokinetics of the non-isotopic analogs. For example, the hydrogen atom at a certain position on the Compounds of the Invention may be replaced with deuterium (a stable isotope which is non-radioactive). Examples of known stable isotopes include, but are not limited to, deuterium (2^{H}), ¹³C, ¹⁵N, ¹⁸O. Alternatively, unstable isotopes, which are radioactive isotopes which contain additional neutrons compared to the abundant nuclides of the same species (i.e., element), e.g., ¹²³I, ¹³¹I, ¹²⁵I, ¹⁴C, ¹⁸F, may replace the corresponding abundant species of I, C and F. Another example of useful isotope of the compound of the invention is the ¹¹C isotope. These radio isotopes are useful for radio-imaging and/or pharmacokinetic studies of the compounds of the invention.

Melting points are uncorrected and (dec) indicates decomposition. Temperatures are given in degrees Celsius (° C.); unless otherwise stated, operations are carried out at room or ambient temperature, that is, at a temperature in the range of 18-25° C.

Chromatography means flash chromatography on silica gel; thin layer chromatography (TLC) is carried out on silica gel plates. NMR data is in the delta values of major diagnostic protons, given in parts per million (ppm) relative to tetramethylsilane (TMS) as an internal standard. Conventional abbreviations for signal shape are used. Coupling constants (J) are given in Hz. For mass spectra (MS), the lowest mass major ion is reported for molecules where isotope splitting results in multiple mass spectral peaks Solvent mixture compositions are given as volume percentages or volume ratios. In cases where the NMR spectra are complex, only diagnostic signals are reported.

The words “treatment” and “treating” are to be understood accordingly as embracing treatment or amelioration of symptoms of disease as well as treatment of the cause of the disease. For methods of treatment, the word “effective amount” is intended to encompass a therapeutically effective amount to treat a specific disease or disorder.

The term “patient” includes human and non-human (i.e., animal) patients. In particular embodiments, the invention encompasses both human and nonhuman patients. In another embodiment, the invention encompasses non-human patients. In other embodiment, the term encompasses human patients.

The term “comprising” as used in this disclosure is intended to be open-ended and does not exclude additional, unrecited elements or method steps.

Compounds of the Invention, e.g., compounds of Formula I or any of formulas 1.1-1.126 as hereinbefore described, in free or pharmaceutically acceptable salt form, may be used as a sole therapeutic agent, but may also be used in combination or for co-administration with other active agents.

Dosages employed in practicing the methods of present invention will of course vary depending, e.g., on the particular disease or condition to be treated, the particular compound used, the mode of administration, and the therapy desired. The compound may be administered by any suitable route, including orally, parenterally, transdermally, or by inhalation, but are preferably administered orally. In general, satisfactory results, e.g., for the treatment of diseases as hereinbefore set forth are indicated to be obtained on oral administration at dosages of the order from about 0.01 to 2.0 mg/kg. In larger mammals, for example humans, an indicated daily dosage for oral administration will accordingly be in the range of from about 0.75 to 1000 mg, conveniently administered once, or in divided doses 2 to 4 times, daily or in sustained release form. Unit dosage forms for oral administration thus for example may comprise from about 0.2 to 75 or 150 mg or 300 mg, e.g., from about 0.2 or 2.0 to 10, 25, 50, 75, 100, 150, 200 or 300 mg of the compound disclosed herein, together with a pharmaceutically acceptable diluent or carrier therefor.

The term “pharmaceutically acceptable diluent or carrier” is intended to mean diluents and carriers that are useful in pharmaceutical preparations, and that are free of substances that are allergenic, pyrogenic or pathogenic, and that are known to potentially cause or promote illness. Pharmaceutically acceptable diluents or carriers thus exclude bodily fluids such as example blood, urine, spinal fluid, saliva, and the like, as well as their constituent components such as blood cells and circulating proteins. Suitable pharmaceutically acceptable diluents and carriers can be found in any of several well-known treatises on pharmaceutical formulations, for example Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remington's Pharmaceutical Sciences, 20^{th} Ed., Lippincott Williams & Wilkins, 2000; and Martindale, The Extra Pharmacopoeia, Thirty-Second Edition (The Pharmaceutical Press, London, 1999); all of which are incorporated by reference herein in their entirety.

All numerical designations, e.g., pH, temperature, time, concentration, molecular weight, including ranges, are approximations which are varied (+) or (−) by increments of 0.1 or 1.0, where appropriate. It is to be understood, although not always explicitly stated, that all numerical designations are preceded by the term “about.” It also is to be understood, although not always explicitly stated, that the reagents described herein are merely exemplary and that equivalents of such are known in the art.

Pharmaceutical compositions comprising Compounds of the Invention may be prepared using conventional diluents or excipients and techniques known in the galenic art. Thus, oral dosage forms may include tablets, capsules, solutions, suspensions and the like.

Methods of Making Compounds of the Invention

The Compounds of the Invention and their pharmaceutically acceptable salts may be made using the methods as described and exemplified herein and/or by methods similar thereto and/or by methods known in the chemical art. Such methods include, but not limited to, those described below. If not commercially available, starting materials for these processes may be made by procedures, which are selected from the chemical art using techniques which are similar or analogous to the synthesis of known compounds.

Compounds of the present disclosure may be prepared according to the general procedures known in the art, including those found in the following references, which are incorporated herein by reference in their entireties: WO 2007/127183, WO 2009/013545, WO 2009/042607, WO 2009/053716, WO 2010/138589, WO 2012/104776, WO2013/076182, WO 2016/157074, WO 2019/113523, WO 2020/123850, WO2020/168927 (Chinese) and US US2022/0056043, WO2021/057256 (Chinese), WO 2023/055181 (Chinese), U.S. Pat. Nos. 7,750,556, and 8,044,068. Further relevant synthetic procedures may be found in Barnes et al., J. Med. Chem. 59: 10402-10422 (2019), Bonazzi et al., J. Med. Chem. 63: 1068-1083 (2020), Chen et al., J. Med. Chem. 59: 5488-5504 (2016), Folkes et al., J. Med. Chem. 51: 5522-5532 (2008), Heffron et al., J. Med. Chem. 55: 8007-8020 (2012), Koehler et al., J. Med. Chem. 55: 10958-10971 (2012), Murray et al., J. Med. Chem. 55: 7686-7695 (2012), Nowak et al., J. Med. Chem. 52: 7081-7089 (2009); Safina et al., J. Med Chem. 55: 5887-5900 (2012), Sutherlin et al., J. Med. Chem. 54: 7579-7587 (2011), and Zhang et al., Bioorg. & Med. Chem. Lett. 27 3395-3398 (2017).

As an example, compounds of the present disclosure having a thieno[3,2-d]pyrimidine core, can be prepared according to the synthetic scheme provided below, and as further shown in the Examples section below.

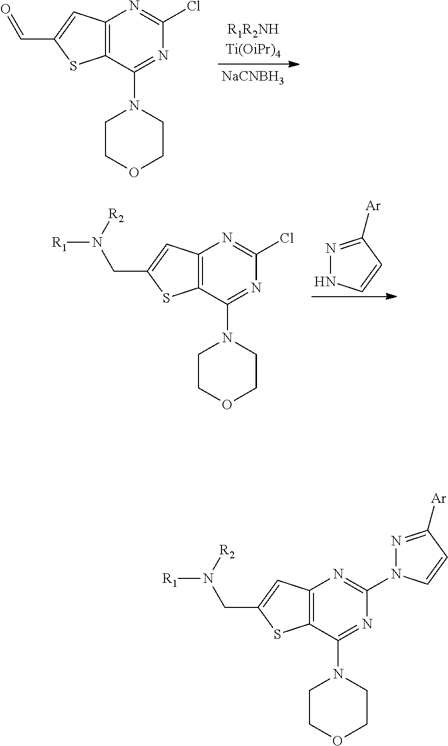

Terms and Abbreviations
AcOH: Acetic acid
ACN: Acetonitrile
dba: dibenzylideneacetone
dppf: 1,1′-Bis(diphenylphosphino)ferrocene
DBU: 1,8-Diazabicyclo[5.4.0]undec-7-ene
DCE: Dichloroethane
DCM: Dichloromethane
DHP: 3,4-Dihydropyran
DIPEA: N,N-diisopropyl-N-ethylamine
DMF: Dimethylformamide
DMSO: Dimethylsulfoxide
DMTMM: 4-(4,6-Dimethoxy[1,3,5]triazin-2-yl)-4-methylmorpholinium chloride
EtOAc: Ethyl acetate
h: hour(s)
HATU: 1-[Bis(dimethylamino)methylene]-1H-1,2,3-triazolo[4,5-b]pyridinium-3-oxide hexafluorophosphate
IPA: Isopropanol
LiHMDS: Lithium hexamethyldisilazide (Lithium bis(trimethylsilyl)amide)
MeOH: Methanol
min: minute(s)
n-BuLi: n-butyllithium
NaOH: Sodium hydroxide
NMR: Nuclear magnetic resonance spectroscopy
PTSA: para-Toluenesulfonic acid
RP: Reverse phase
RT: Room temperature
t-BuXPhos: 2-Di-tert-butylphosphino-2′,4′,6′-triisopropylbiphenyl
TBSCl: tert-butyldimethylsilyl chloride
TBAF: Tetra-n-butylammonium fluoride
TEA: Triethylamine
T3P: Propylphosphonic anhydride
THF: Tetrahydrofuran
TLC: Thin-layer chromatography

### EXAMPLES

The compounds of Table 1 and Table 2 are or can be prepared according to the synthetic methods detailed hereinbelow.

TABLE 1

| Ex. No. | Structure |
|---|---|
| 1 | 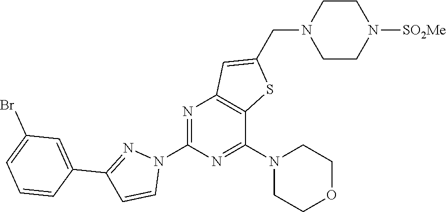 [SMILES: CS(=O)(=O)N1CCN(Cc2cc3nc(-n4ccc(-c5cccc(Br)c5)n4)nc(N4CCOCC4)c3s2)CC1] |
| 2 | 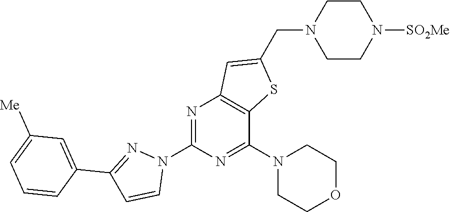 [SMILES: Cc1cccc(-c2ccn(-c3nc(N4CCOCC4)c4sc(CN5CCN(S(C)(=O)=O)CC5)cc4n3)n2)c1] |
| 3 | 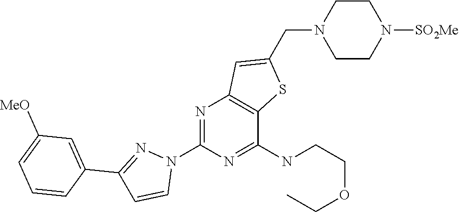 [SMILES: CCOCCNc1nc(-n2ccc(-c3cccc(OC)c3)n2)nc2cc(CN3CCN(S(C)(=O)=O)CC3)sc12] |
| 4 | 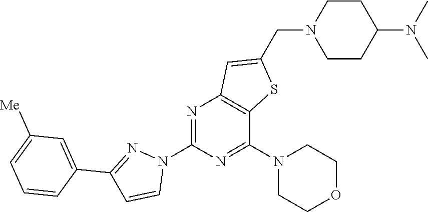 [SMILES: Cc1cccc(-c2ccn(-c3nc(N4CCOCC4)c4sc(CN5CCC(N(C)C)CC5)cc4n3)n2)c1] |
| 5 | 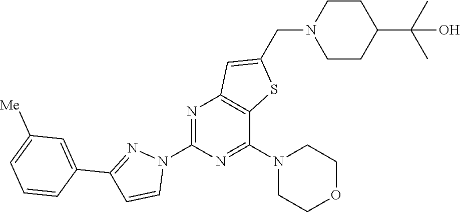 [SMILES: Cc1cccc(-c2ccn(-c3nc(N4CCOCC4)c4sc(CN5CCC(C(C)(C)O)CC5)cc4n3)n2)c1] |
| 6 | 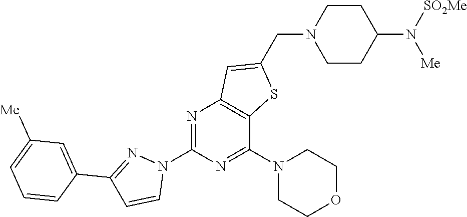 [SMILES: Cc1cccc(-c2ccn(-c3nc(N4CCOCC4)c4sc(CN5CCC(N(C)S(C)(=O)=O)CC5)cc4n3)n2)c1] |
| 7 | 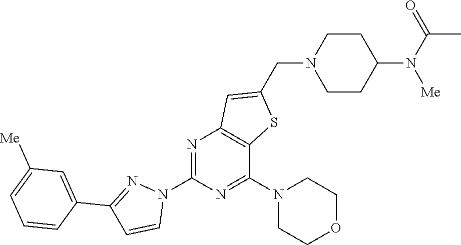 [SMILES: CC(=O)N(C)C1CCN(Cc2cc3nc(-n4ccc(-c5cccc(C)c5)n4)nc(N4CCOCC4)c3s2)CC1] |
| 8 | 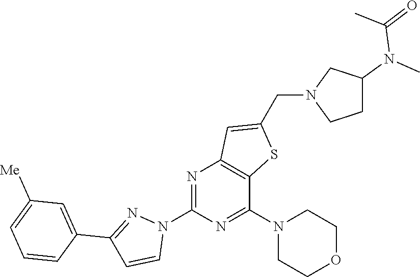 [SMILES: CC(=O)N(C)C1CCN(Cc2cc3nc(-n4ccc(-c5cccc(C)c5)n4)nc(N4CCOCC4)c3s2)C1] |
| 9 | 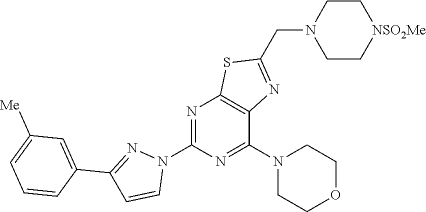 |
| 10 | 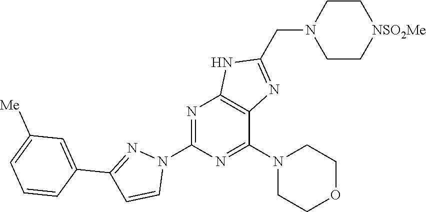 |
| 11 | 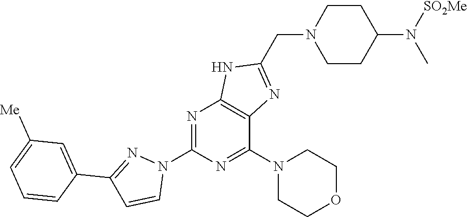 [SMILES: Cc1cccc(-c2ccn(-c3nc(N4CCOCC4)c4nc(CN5CCC(N(C)S(C)(=O)=O)CC5)[nH]c4n3)n2)c1] |
| 12 | 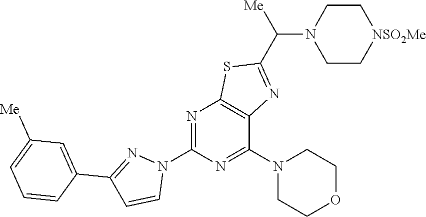 |
| 13 | 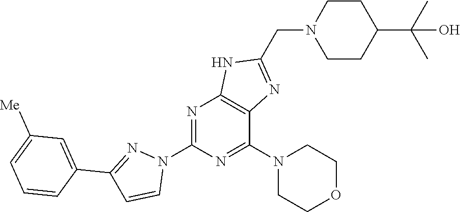 [SMILES: Cc1cccc(-c2ccn(-c3nc(N4CCOCC4)c4nc(CN5CCC(C(C)(C)O)CC5)[nH]c4n3)n2)c1] |
| 14 | 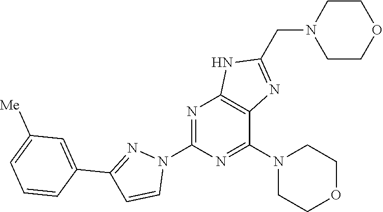 [SMILES: Cc1cccc(-c2ccn(-c3nc(N4CCOCC4)c4nc(CN5CCOCC5)[nH]c4n3)n2)c1] |
| 15 | 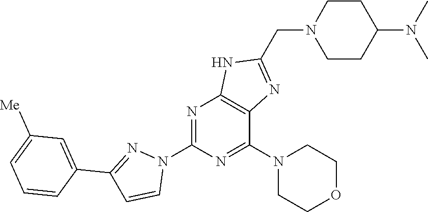 [SMILES: Cc1cccc(-c2ccn(-c3nc(N4CCOCC4)c4nc(CN5CCC(N(C)C)CC5)[nH]c4n3)n2)c1] |
| 16 | 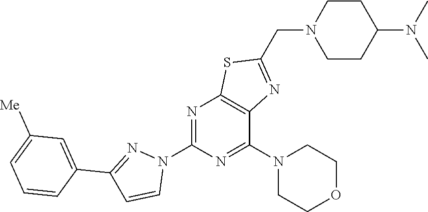 [SMILES: Cc1cccc(-c2ccn(-c3nc(N4CCOCC4)c4nc(CN5CCC(N(C)C)CC5)sc4n3)n2)c1] |
| 17 | 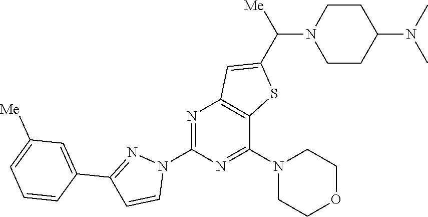 [SMILES: Cc1cccc(-c2ccn(-c3nc(N4CCOCC4)c4sc(C(C)N5CCC(N(C)C)CC5)cc4n3)n2)c1] |
| 18 | 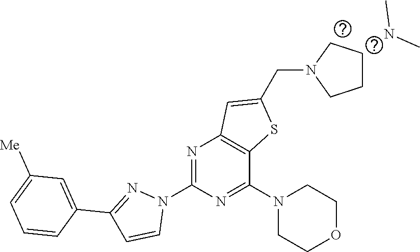 |
| 19 | 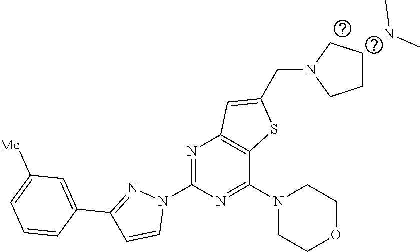 |
| 20 | 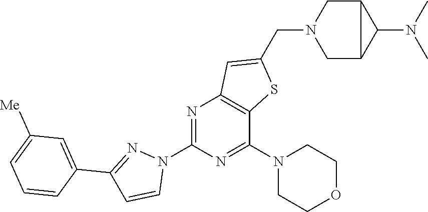 [SMILES: Cc1cccc(-c2ccn(-c3nc(N4CCOCC4)c4sc(CN5CC6C(C5)C6N(C)C)cc4n3)n2)c1] |
| 21 | 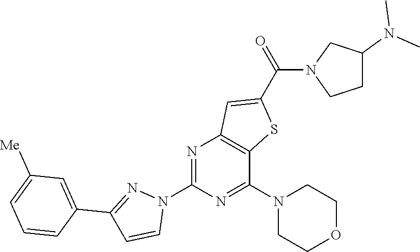 [SMILES: Cc1cccc(-c2ccn(-c3nc(N4CCOCC4)c4sc(C(=O)N5CCC(N(C)C)C5)cc4n3)n2)c1] |
| 22 | 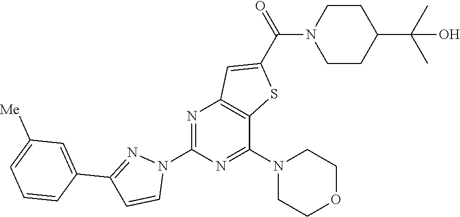 [SMILES: Cc1cccc(-c2ccn(-c3nc(N4CCOCC4)c4sc(C(=O)N5CCC(C(C)(C)O)CC5)cc4n3)n2)c1] |
| 23 | 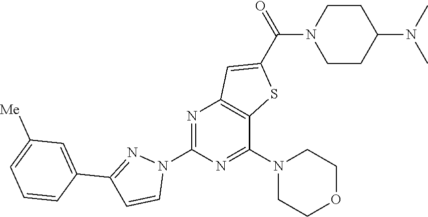 [SMILES: Cc1cccc(-c2ccn(-c3nc(N4CCOCC4)c4sc(C(=O)N5CCC(N(C)C)CC5)cc4n3)n2)c1] |
| 24 | 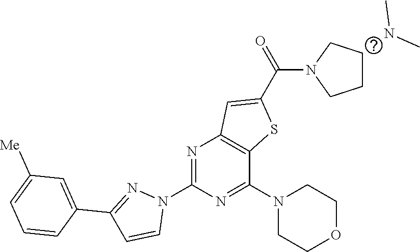 |
| 25 | 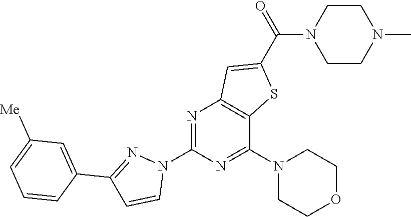 [SMILES: Cc1cccc(-c2ccn(-c3nc(N4CCOCC4)c4sc(C(=O)N5CCN(C)CC5)cc4n3)n2)c1] |
| 26 | 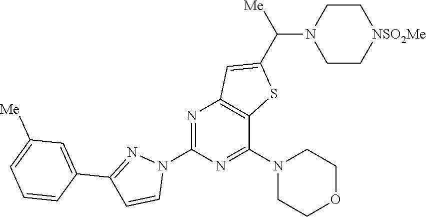 |
| 27 | 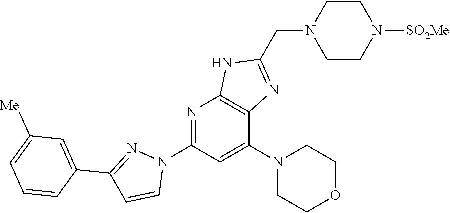 [SMILES: Cc1cccc(-c2ccn(-c3cc(N4CCOCC4)c4nc(CN5CCN(S(C)(=O)=O)CC5)[nH]c4n3)n2)c1] |
| 28 | 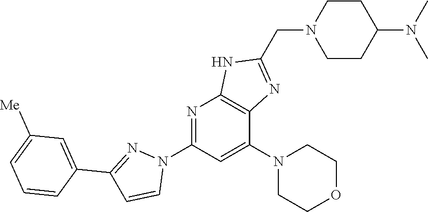 [SMILES: Cc1cccc(-c2ccn(-c3cc(N4CCOCC4)c4nc(CN5CCC(N(C)C)CC5)[nH]c4n3)n2)c1] |
| 29 | 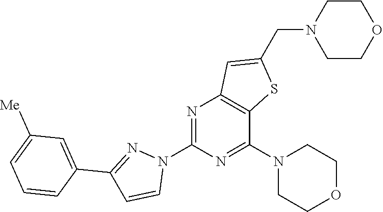 [SMILES: Cc1cccc(-c2ccn(-c3nc(N4CCOCC4)c4sc(CN5CCOCC5)cc4n3)n2)c1] |
| 30 | 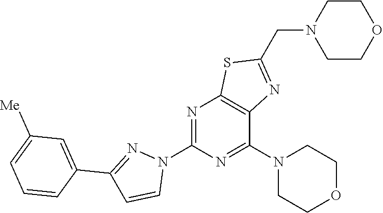 [SMILES: Cc1cccc(-c2ccn(-c3nc(N4CCOCC4)c4nc(CN5CCOCC5)sc4n3)n2)c1] |
| 31 | 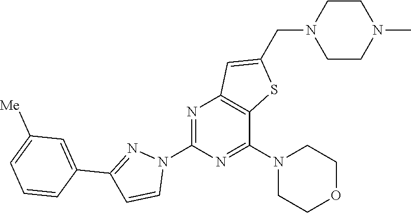 [SMILES: Cc1cccc(-c2ccn(-c3nc(N4CCOCC4)c4sc(CN5CCN(C)CC5)cc4n3)n2)c1] |
| 32 | 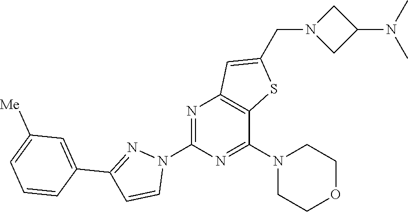 [SMILES: Cc1cccc(-c2ccn(-c3nc(N4CCOCC4)c4sc(CN5CC(N(C)C)C5)cc4n3)n2)c1] |
| 33 | 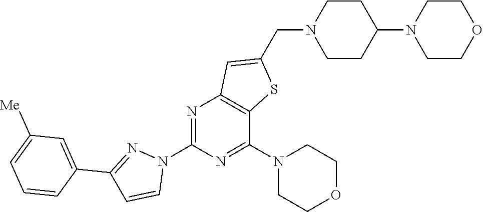 [SMILES: Cc1cccc(-c2ccn(-c3nc(N4CCOCC4)c4sc(CN5CCC(N6CCOCC6)CC5)cc4n3)n2)c1] |
| 34 | 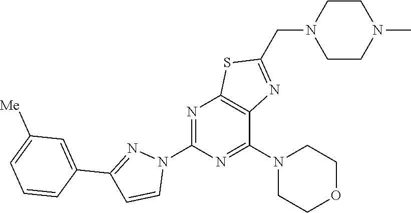 [SMILES: Cc1cccc(-c2ccn(-c3nc(N4CCOCC4)c4nc(CN5CCN(C)CC5)sc4n3)n2)c1] |
| 35 | 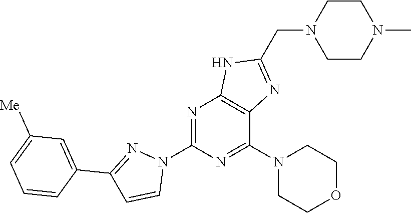 [SMILES: Cc1cccc(-c2ccn(-c3nc(N4CCOCC4)c4nc(CN5CCN(C)CC5)[nH]c4n3)n2)c1] |
| 36 | 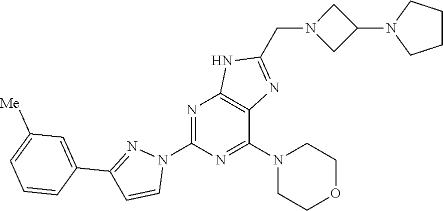 [SMILES: Cc1cccc(-c2ccn(-c3nc(N4CCOCC4)c4nc(CN5CC(N6CCCC6)C5)[nH]c4n3)n2)c1] |
| 37 | 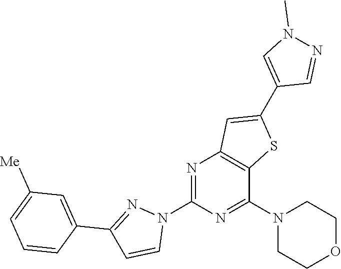 [SMILES: Cc1cccc(-c2ccn(-c3nc(N4CCOCC4)c4sc(-c5cnn(C)c5)cc4n3)n2)c1] |
| 38 | 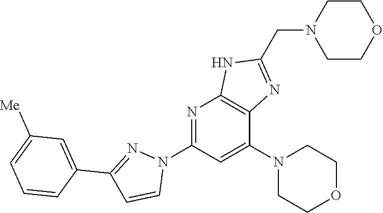 [SMILES: Cc1cccc(-c2ccn(-c3cc(N4CCOCC4)c4nc(CN5CCOCC5)[nH]c4n3)n2)c1] |
| 39 | 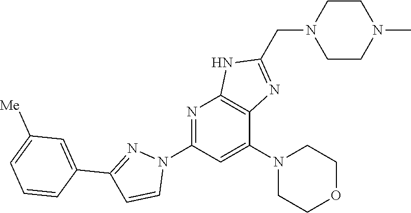 [SMILES: Cc1cccc(-c2ccn(-c3cc(N4CCOCC4)c4nc(CN5CCN(C)CC5)[nH]c4n3)n2)c1] |
| 40 | 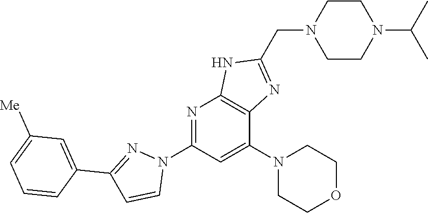 [SMILES: Cc1cccc(-c2ccn(-c3cc(N4CCOCC4)c4nc(CN5CCN(C(C)C)CC5)[nH]c4n3)n2)c1] |
| 41 | 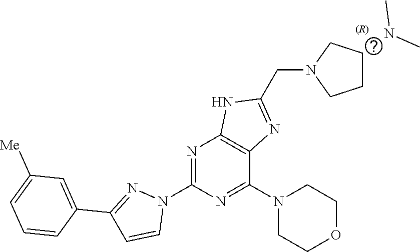 |
| 42 | 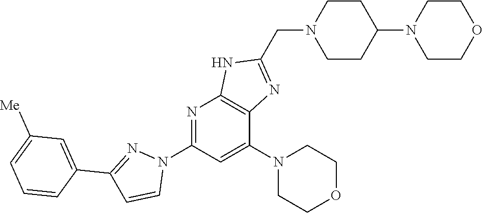 [SMILES: Cc1cccc(-c2ccn(-c3cc(N4CCOCC4)c4nc(CN5CCC(N6CCOCC6)CC5)[nH]c4n3)n2)c1] |
| 43 | 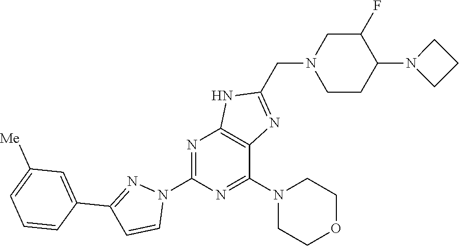 [SMILES: Cc1cccc(-c2ccn(-c3nc(N4CCOCC4)c4nc(CN5CCC(N6CCC6)C(F)C5)[nH]c4n3)n2)c1] |
| 44 | 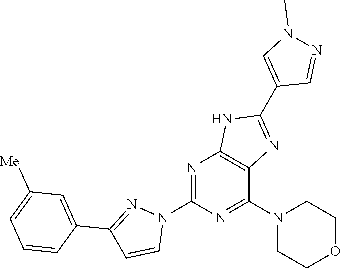 [SMILES: Cc1cccc(-c2ccn(-c3nc(N4CCOCC4)c4nc(-c5cnn(C)c5)[nH]c4n3)n2)c1] |
| 45 | 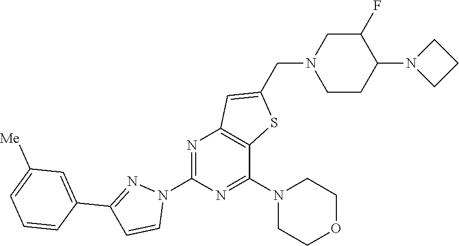 [SMILES: Cc1cccc(-c2ccn(-c3nc(N4CCOCC4)c4sc(CN5CCC(N6CCC6)C(F)C5)cc4n3)n2)c1] |
| 46 | 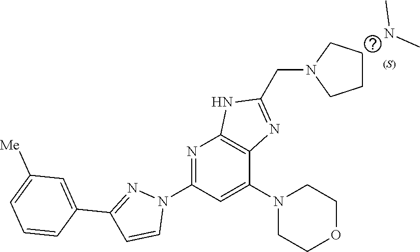 |
| 47 | 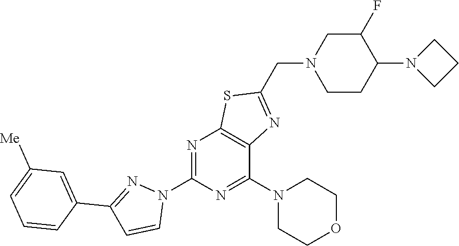 [SMILES: Cc1cccc(-c2ccn(-c3nc(N4CCOCC4)c4nc(CN5CCC(N6CCC6)C(F)C5)sc4n3)n2)c1] |
| 48 | 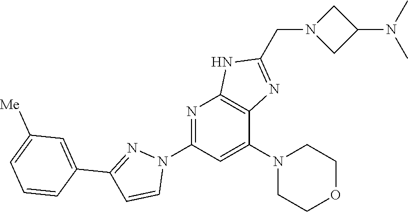 [SMILES: Cc1cccc(-c2ccn(-c3cc(N4CCOCC4)c4nc(CN5CC(N(C)C)C5)[nH]c4n3)n2)c1] |
| 49 | 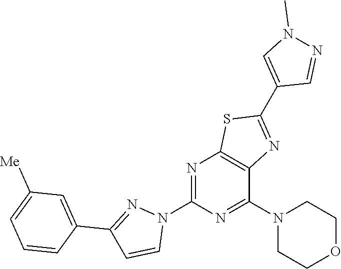 [SMILES: Cc1cccc(-c2ccn(-c3nc(N4CCOCC4)c4nc(-c5cnn(C)c5)sc4n3)n2)c1] |
| 50 | 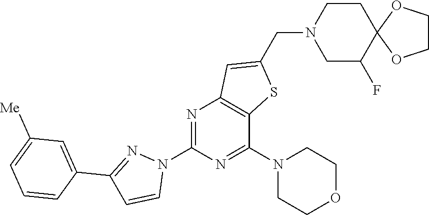 [SMILES: Cc1cccc(-c2ccn(-c3nc(N4CCOCC4)c4sc(CN5CCC6(OCCO6)C(F)C5)cc4n3)n2)c1] |
| 51 | 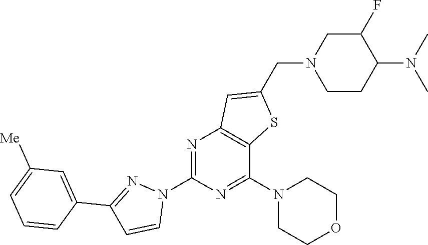 [SMILES: Cc1cccc(-c2ccn(-c3nc(N4CCOCC4)c4sc(CN5CCC(N(C)C)C(F)C5)cc4n3)n2)c1] |
| 52 | 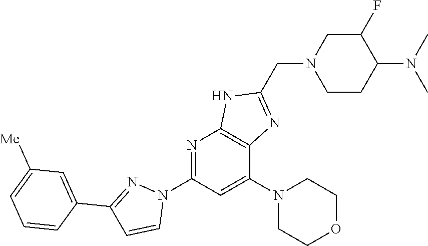 [SMILES: Cc1cccc(-c2ccn(-c3cc(N4CCOCC4)c4nc(CN5CCC(N(C)C)C(F)C5)[nH]c4n3)n2)c1] |
| 53 | 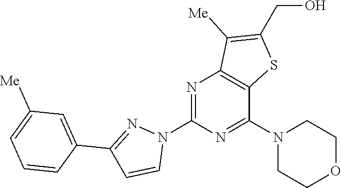 [SMILES: Cc1cccc(-c2ccn(-c3nc(N4CCOCC4)c4sc(CO)c(C)c4n3)n2)c1] |
| 54 | 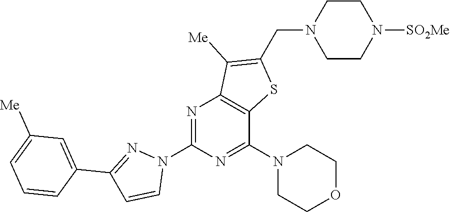 [SMILES: Cc1cccc(-c2ccn(-c3nc(N4CCOCC4)c4sc(CN5CCN(S(C)(=O)=O)CC5)c(C)c4n3)n2)c1] |
| 55 | 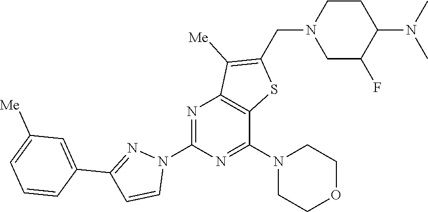 [SMILES: Cc1cccc(-c2ccn(-c3nc(N4CCOCC4)c4sc(CN5CCC(N(C)C)C(F)C5)c(C)c4n3)n2)c1] |
| 56 | 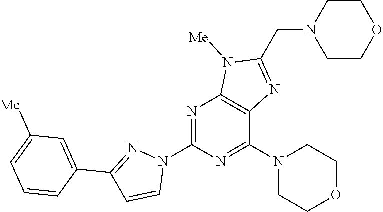 [SMILES: Cc1cccc(-c2ccn(-c3nc(N4CCOCC4)c4nc(CN5CCOCC5)n(C)c4n3)n2)c1] |
| 57 | 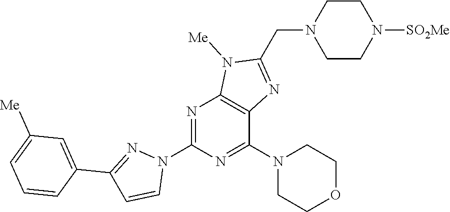 [SMILES: Cc1cccc(-c2ccn(-c3nc(N4CCOCC4)c4nc(CN5CCN(S(C)(=O)=O)CC5)n(C)c4n3)n2)c1] |
| 58 | 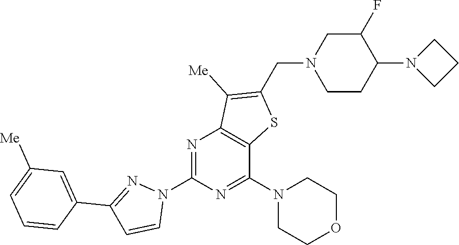 [SMILES: Cc1cccc(-c2ccn(-c3nc(N4CCOCC4)c4sc(CN5CCC(N6CCC6)C(F)C5)c(C)c4n3)n2)c1] |
| 59 | 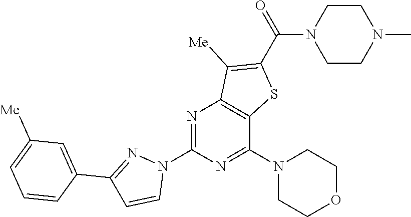 [SMILES: Cc1cccc(-c2ccn(-c3nc(N4CCOCC4)c4sc(C(=O)N5CCN(C)CC5)c(C)c4n3)n2)c1] |
| 60 | 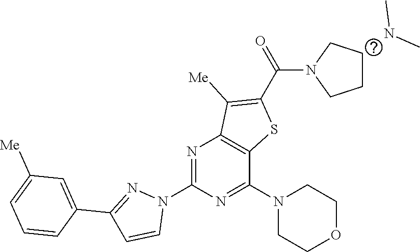 |
| 61 | 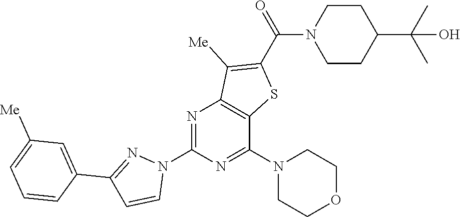 [SMILES: Cc1cccc(-c2ccn(-c3nc(N4CCOCC4)c4sc(C(=O)N5CCC(C(C)(C)O)CC5)c(C)c4n3)n2)c1] |
| 62 | 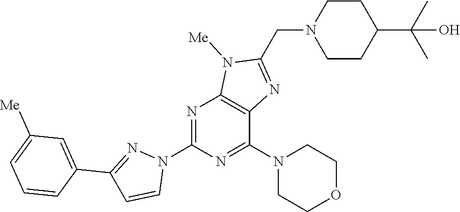 [SMILES: Cc1cccc(-c2ccn(-c3nc(N4CCOCC4)c4nc(CN5CCC(C(C)(C)O)CC5)n(C)c4n3)n2)c1] |
| 63 | 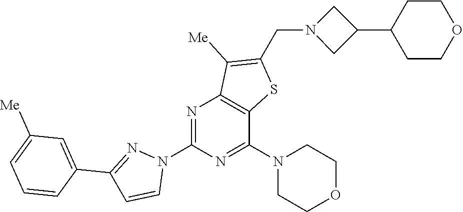 [SMILES: Cc1cccc(-c2ccn(-c3nc(N4CCOCC4)c4sc(CN5CC(C6CCOCC6)C5)c(C)c4n3)n2)c1] |
| 64 | 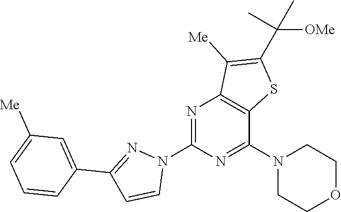 [SMILES: COC(C)(C)c1sc2c(N3CCOCC3)nc(-n3ccc(-c4cccc(C)c4)n3)nc2c1C] |
| 65 | 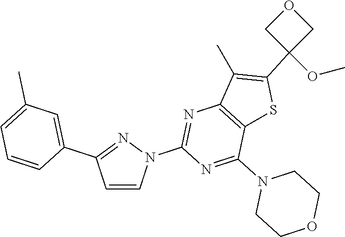 [SMILES: COC1(c2sc3c(N4CCOCC4)nc(-n4ccc(-c5cccc(C)c5)n4)nc3c2C)COC1] |
| 66 | 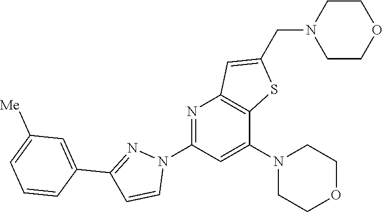 [SMILES: Cc1cccc(-c2ccn(-c3cc(N4CCOCC4)c4sc(CN5CCOCC5)cc4n3)n2)c1] |
| 67 | 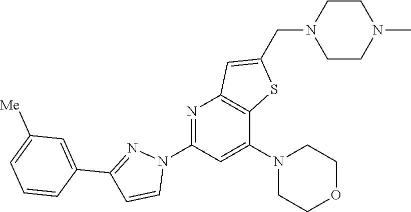 [SMILES: Cc1cccc(-c2ccn(-c3cc(N4CCOCC4)c4sc(CN5CCN(C)CC5)cc4n3)n2)c1] |
| 68 | 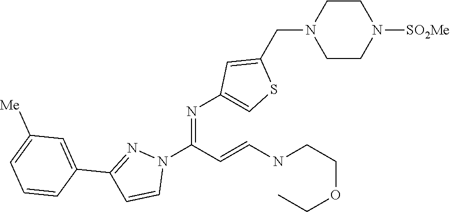 [SMILES: CCOCCN/C=C/C(=N\c1csc(CN2CCN(S(C)(=O)=O)CC2)c1)n1ccc(-c2cccc(C)c2)n1] |
| 69 | 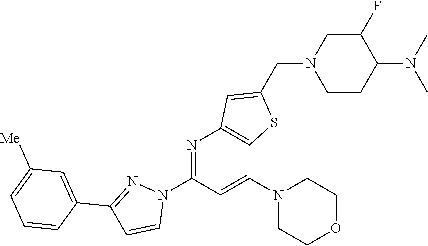 [SMILES: Cc1cccc(-c2ccn(C(/C=C/N3CCOCC3)=N/c3csc(CN4CCC(N(C)C)C(F)C4)c3)n2)c1] |
| 70 | 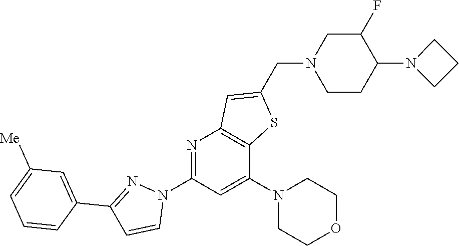 [SMILES: Cc1cccc(-c2ccn(-c3cc(N4CCOCC4)c4sc(CN5CCC(N6CCC6)C(F)C5)cc4n3)n2)c1] |
| 71 | 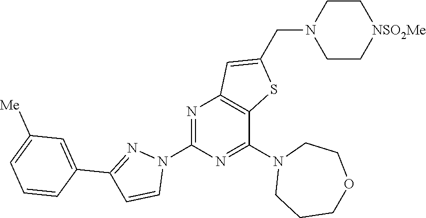 |
| 72 | 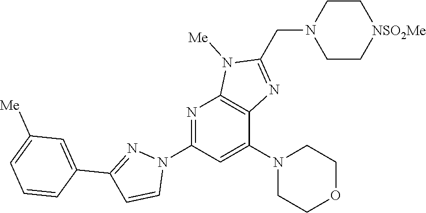 |
| 73 | 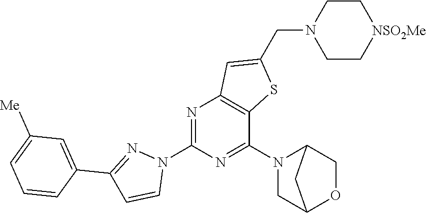 |
| 74 | 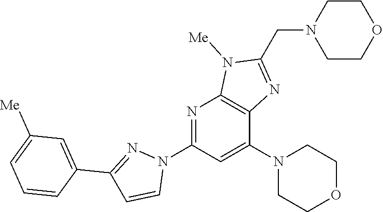 [SMILES: Cc1cccc(-c2ccn(-c3cc(N4CCOCC4)c4nc(CN5CCOCC5)n(C)c4n3)n2)c1] |
| 75 | 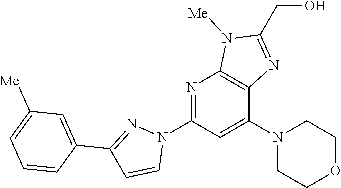 [SMILES: Cc1cccc(-c2ccn(-c3cc(N4CCOCC4)c4nc(CO)n(C)c4n3)n2)c1] |
| 76 | 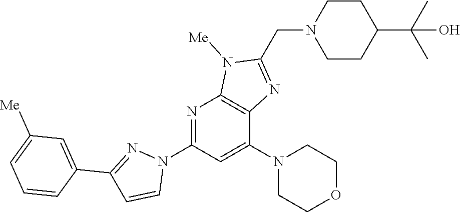 [SMILES: Cc1cccc(-c2ccn(-c3cc(N4CCOCC4)c4nc(CN5CCC(C(C)(C)O)CC5)n(C)c4n3)n2)c1] |
| 77 | 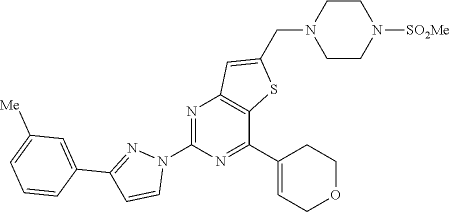 [SMILES: Cc1cccc(-c2ccn(-c3nc(C4=CCOCC4)c4sc(CN5CCN(S(C)(=O)=O)CC5)cc4n3)n2)c1] |
| 78 | 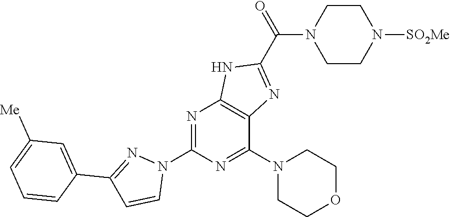 [SMILES: Cc1cccc(-c2ccn(-c3nc(N4CCOCC4)c4nc(C(=O)N5CCN(S(C)(=O)=O)CC5)[nH]c4n3)n2)c1] |
| 79 | 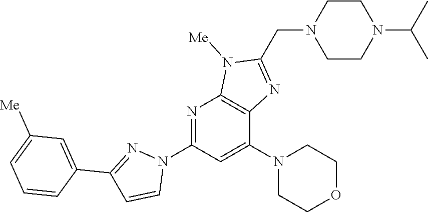 [SMILES: Cc1cccc(-c2ccn(-c3cc(N4CCOCC4)c4nc(CN5CCN(C(C)C)CC5)n(C)c4n3)n2)c1] |
| 82 | 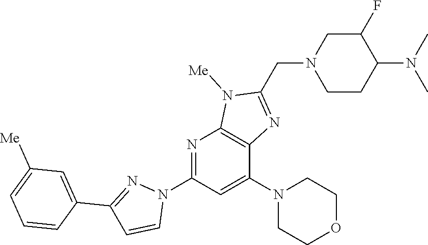 [SMILES: Cc1cccc(-c2ccn(-c3cc(N4CCOCC4)c4nc(CN5CCC(N(C)C)C(F)C5)n(C)c4n3)n2)c1] |
| 83 | 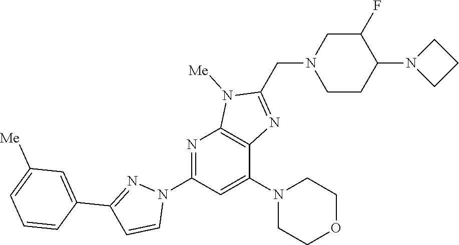 [SMILES: Cc1cccc(-c2ccn(-c3cc(N4CCOCC4)c4nc(CN5CCC(N6CCC6)C(F)C5)n(C)c4n3)n2)c1] |
| 84 | 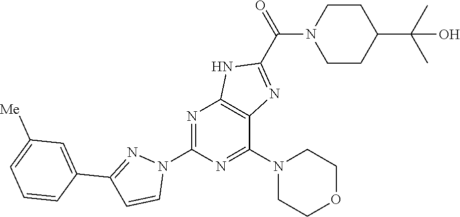 [SMILES: Cc1cccc(-c2ccn(-c3nc(N4CCOCC4)c4nc(C(=O)N5CCC(C(C)(C)O)CC5)[nH]c4n3)n2)c1] |
| 85 | 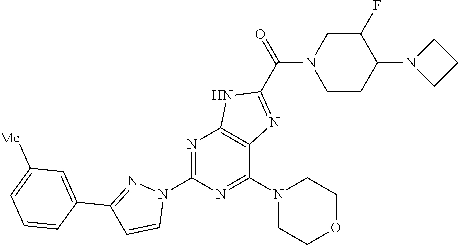 [SMILES: Cc1cccc(-c2ccn(-c3nc(N4CCOCC4)c4nc(C(=O)N5CCC(N6CCC6)C(F)C5)[nH]c4n3)n2)c1] |
| 86 | 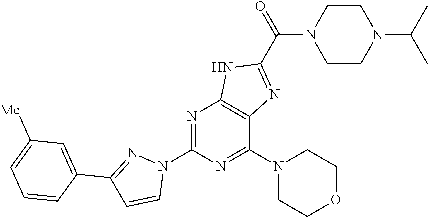 [SMILES: Cc1cccc(-c2ccn(-c3nc(N4CCOCC4)c4nc(C(=O)N5CCN(C(C)C)CC5)[nH]c4n3)n2)c1] |
| 129 | 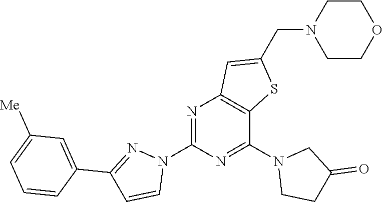 [SMILES: Cc1cccc(-c2ccn(-c3nc(N4CCC(=O)C4)c4sc(CN5CCOCC5)cc4n3)n2)c1] |
| 132 | 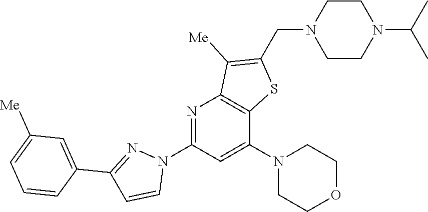 [SMILES: Cc1cccc(-c2ccn(-c3cc(N4CCOCC4)c4sc(CN5CCN(C(C)C)CC5)c(C)c4n3)n2)c1] |
| 165 | 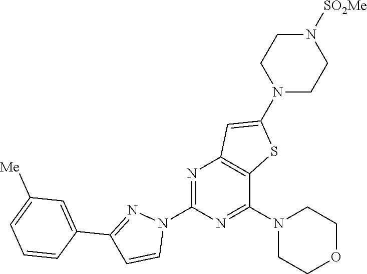 [SMILES: Cc1cccc(-c2ccn(-c3nc(N4CCOCC4)c4sc(N5CCN(S(C)(=O)=O)CC5)cc4n3)n2)c1] |
| 166 | 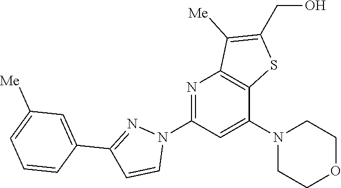 [SMILES: Cc1cccc(-c2ccn(-c3cc(N4CCOCC4)c4sc(CO)c(C)c4n3)n2)c1] |
| 167 | 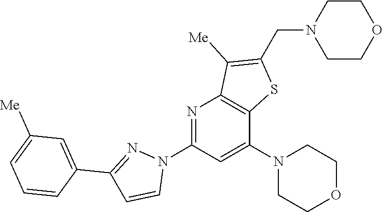 [SMILES: Cc1cccc(-c2ccn(-c3cc(N4CCOCC4)c4sc(CN5CCOCC5)c(C)c4n3)n2)c1] |
| 168 | 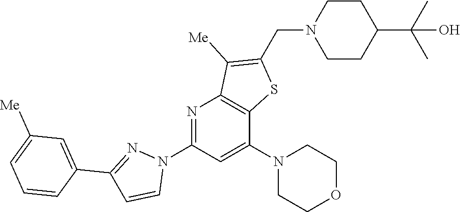 [SMILES: Cc1cccc(-c2ccn(-c3cc(N4CCOCC4)c4sc(CN5CCC(C(C)(C)O)CC5)c(C)c4n3)n2)c1] |
| 169 | 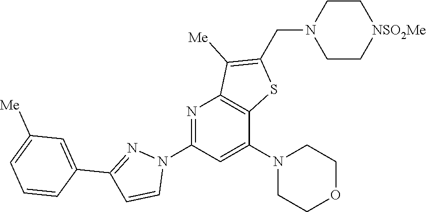 |
| 170 | 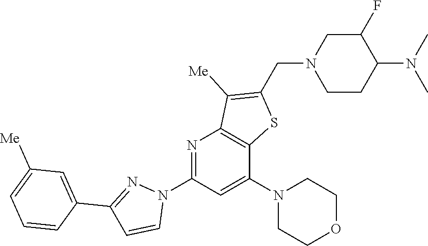 [SMILES: Cc1cccc(-c2ccn(-c3cc(N4CCOCC4)c4sc(CN5CCC(N(C)C)C(F)C5)c(C)c4n3)n2)c1] |
| 171 | 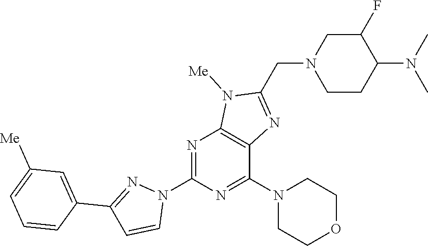 [SMILES: Cc1cccc(-c2ccn(-c3nc(N4CCOCC4)c4nc(CN5CCC(N(C)C)C(F)C5)n(C)c4n3)n2)c1] |
| 172 | 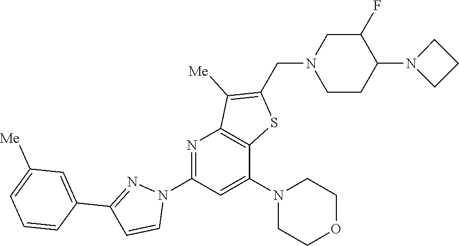 [SMILES: Cc1cccc(-c2ccn(-c3cc(N4CCOCC4)c4sc(CN5CCC(N6CCC6)C(F)C5)c(C)c4n3)n2)c1] |
| 173 | 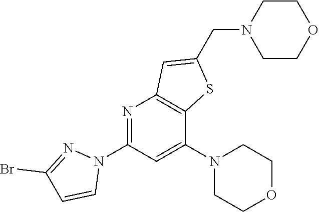 [SMILES: Brc1ccn(-c2cc(N3CCOCC3)c3sc(CN4CCOCC4)cc3n2)n1] |
| 174 | 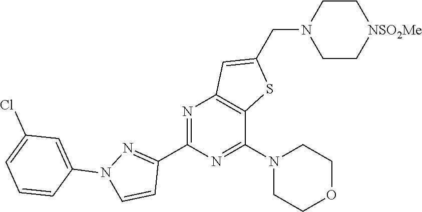 |
| 175 | 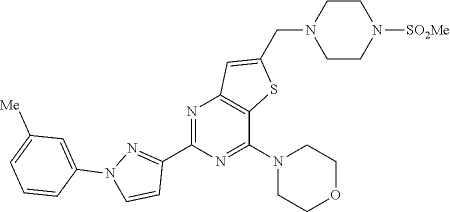 [SMILES: Cc1cccc(-n2ccc(-c3nc(N4CCOCC4)c4sc(CN5CCN(S(C)(=O)=O)CC5)cc4n3)n2)c1] |
| 176 | 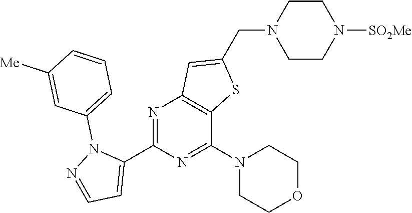 [SMILES: Cc1cccc(-n2nccc2-c2nc(N3CCOCC3)c3sc(CN4CCN(S(C)(=O)=O)CC4)cc3n2)c1] |
| 177 | 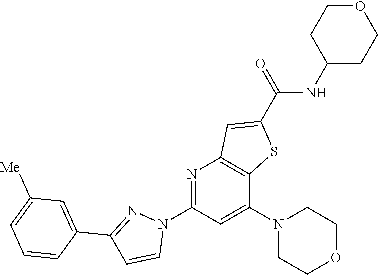 [SMILES: Cc1cccc(-c2ccn(-c3cc(N4CCOCC4)c4sc(C(=O)NC5CCOCC5)cc4n3)n2)c1] |
| 178 | 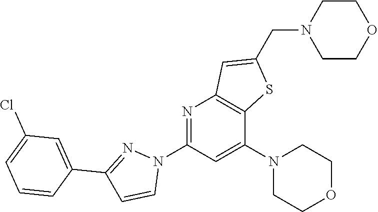 [SMILES: Clc1cccc(-c2ccn(-c3cc(N4CCOCC4)c4sc(CN5CCOCC5)cc4n3)n2)c1] |
| 179 | 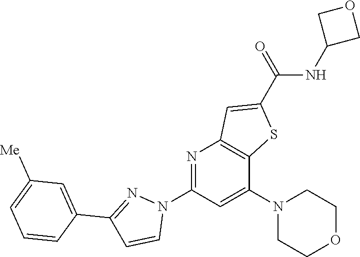 [SMILES: Cc1cccc(-c2ccn(-c3cc(N4CCOCC4)c4sc(C(=O)NC5COC5)cc4n3)n2)c1] |
| 181 | 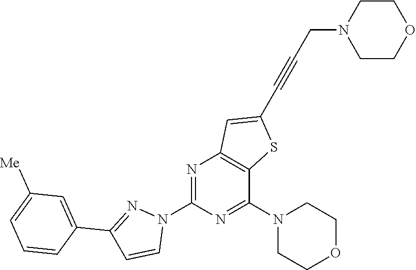 [SMILES: Cc1cccc(-c2ccn(-c3nc(N4CCOCC4)c4sc(C#CCN5CCOCC5)cc4n3)n2)c1] |
| 182 | 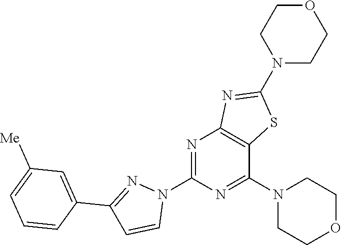 [SMILES: Cc1cccc(-c2ccn(-c3nc(N4CCOCC4)c4sc(N5CCOCC5)nc4n3)n2)c1] |
| 183 | 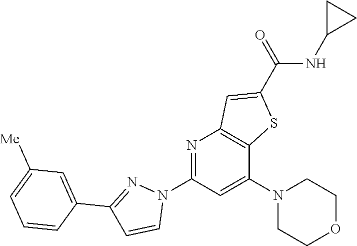 [SMILES: Cc1cccc(-c2ccn(-c3cc(N4CCOCC4)c4sc(C(=O)NC5CC5)cc4n3)n2)c1] |
| 184 | 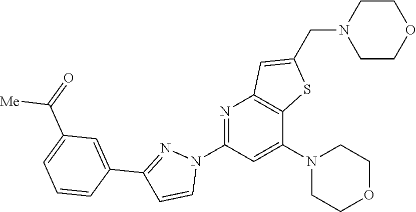 [SMILES: CC(=O)c1cccc(-c2ccn(-c3cc(N4CCOCC4)c4sc(CN5CCOCC5)cc4n3)n2)c1] |
| 185 | 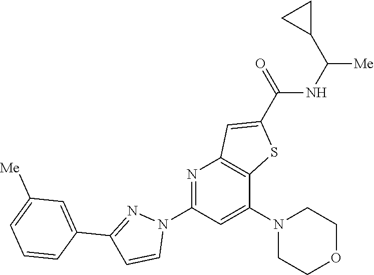 [SMILES: Cc1cccc(-c2ccn(-c3cc(N4CCOCC4)c4sc(C(=O)NC(C)C5CC5)cc4n3)n2)c1] |
| 186 | 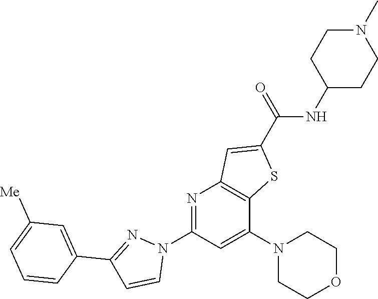 [SMILES: Cc1cccc(-c2ccn(-c3cc(N4CCOCC4)c4sc(C(=O)NC5CCN(C)CC5)cc4n3)n2)c1] |
| 187 | 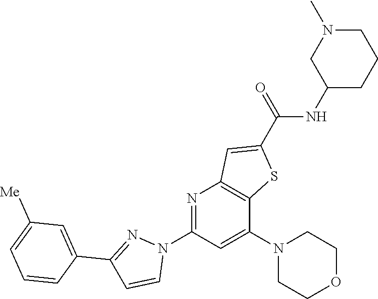 [SMILES: Cc1cccc(-c2ccn(-c3cc(N4CCOCC4)c4sc(C(=O)NC5CCCN(C)C5)cc4n3)n2)c1] |
| 188 | 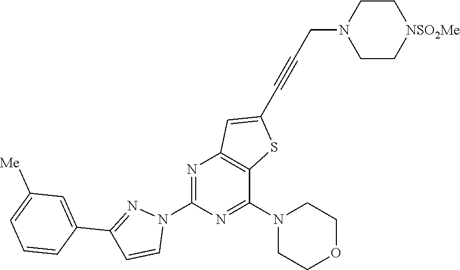 |
| 189 | 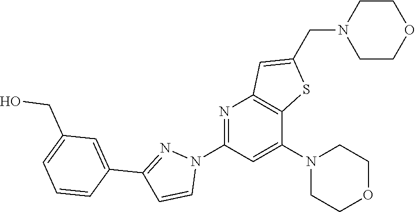 [SMILES: OCc1cccc(-c2ccn(-c3cc(N4CCOCC4)c4sc(CN5CCOCC5)cc4n3)n2)c1] |
| 190 | 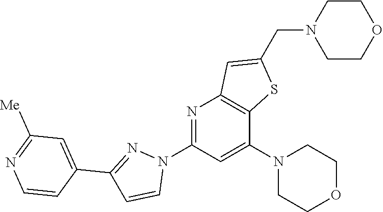 [SMILES: Cc1cc(-c2ccn(-c3cc(N4CCOCC4)c4sc(CN5CCOCC5)cc4n3)n2)ccn1] |
| 191 | 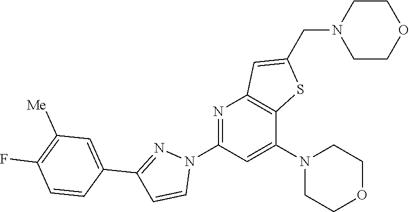 [SMILES: Cc1cc(-c2ccn(-c3cc(N4CCOCC4)c4sc(CN5CCOCC5)cc4n3)n2)ccc1F] |
| 192 | 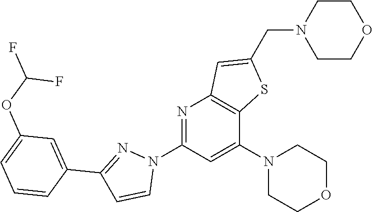 [SMILES: FC(F)Oc1cccc(-c2ccn(-c3cc(N4CCOCC4)c4sc(CN5CCOCC5)cc4n3)n2)c1] |
| 193 | 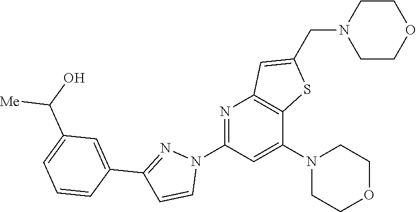 [SMILES: CC(O)c1cccc(-c2ccn(-c3cc(N4CCOCC4)c4sc(CN5CCOCC5)cc4n3)n2)c1] |
| 194 | 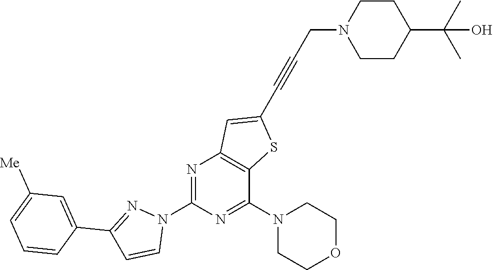 [SMILES: Cc1cccc(-c2ccn(-c3nc(N4CCOCC4)c4sc(C#CCN5CCC(C(C)(C)O)CC5)cc4n3)n2)c1] |
| 195 | 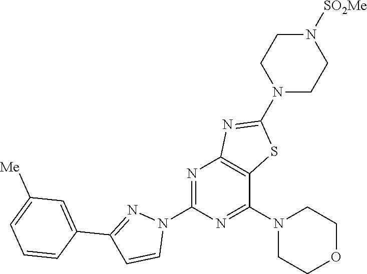 [SMILES: Cc1cccc(-c2ccn(-c3nc(N4CCOCC4)c4sc(N5CCN(S(C)(=O)=O)CC5)nc4n3)n2)c1] |
| 196 | 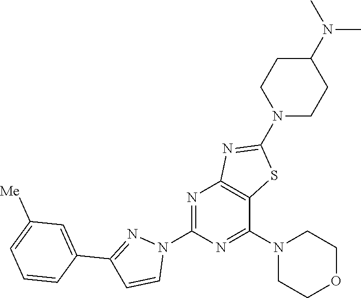 [SMILES: Cc1cccc(-c2ccn(-c3nc(N4CCOCC4)c4sc(N5CCC(N(C)C)CC5)nc4n3)n2)c1] |
| 197 | 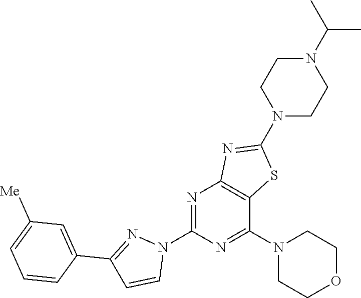 [SMILES: Cc1cccc(-c2ccn(-c3nc(N4CCOCC4)c4sc(N5CCN(C(C)C)CC5)nc4n3)n2)c1] |
| 198 | 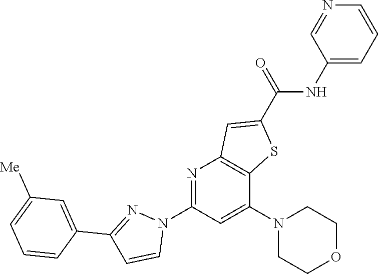 [SMILES: Cc1cccc(-c2ccn(-c3cc(N4CCOCC4)c4sc(C(=O)Nc5cccnc5)cc4n3)n2)c1] |
| 199 | 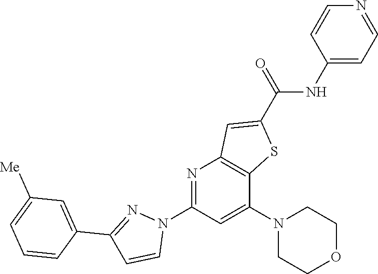 [SMILES: Cc1cccc(-c2ccn(-c3cc(N4CCOCC4)c4sc(C(=O)Nc5ccncc5)cc4n3)n2)c1] |
| 200 | 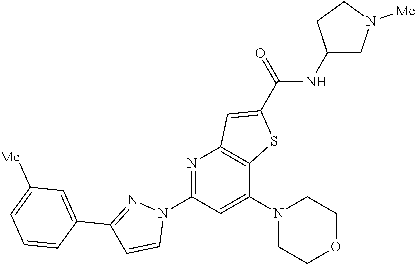 [SMILES: Cc1cccc(-c2ccn(-c3cc(N4CCOCC4)c4sc(C(=O)NC5CCN(C)C5)cc4n3)n2)c1] |
| 201 | 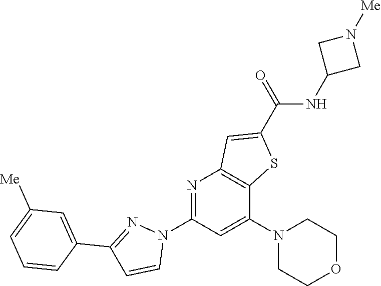 [SMILES: Cc1cccc(-c2ccn(-c3cc(N4CCOCC4)c4sc(C(=O)NC5CN(C)C5)cc4n3)n2)c1] |
| 202 | 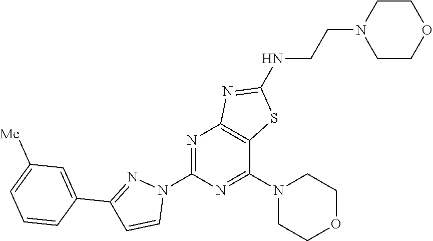 [SMILES: Cc1cccc(-c2ccn(-c3nc(N4CCOCC4)c4sc(NCCN5CCOCC5)nc4n3)n2)c1] |
| 203 | 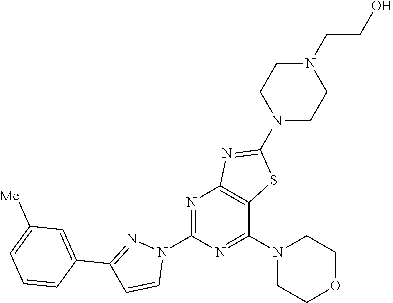 [SMILES: Cc1cccc(-c2ccn(-c3nc(N4CCOCC4)c4sc(N5CCN(CCO)CC5)nc4n3)n2)c1] |
| 204 | 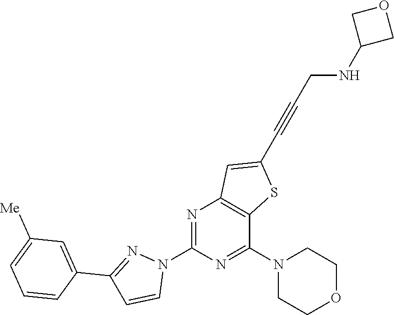 [SMILES: Cc1cccc(-c2ccn(-c3nc(N4CCOCC4)c4sc(C#CCNC5COC5)cc4n3)n2)c1] |
| 205 | 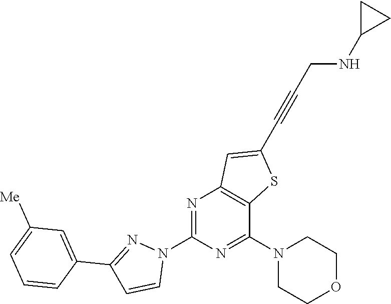 [SMILES: Cc1cccc(-c2ccn(-c3nc(N4CCOCC4)c4sc(C#CCNC5CC5)cc4n3)n2)c1] |
| 206 | 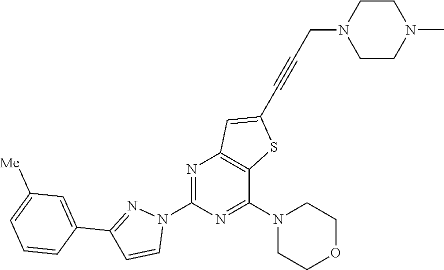 [SMILES: Cc1cccc(-c2ccn(-c3nc(N4CCOCC4)c4sc(C#CCN5CCN(C)CC5)cc4n3)n2)c1] |
| 207 | 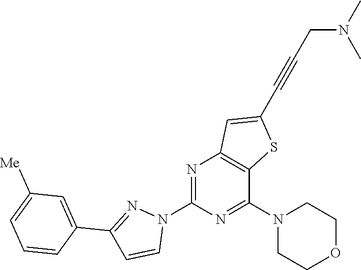 [SMILES: Cc1cccc(-c2ccn(-c3nc(N4CCOCC4)c4sc(C#CCN(C)C)cc4n3)n2)c1] |
| 208 | 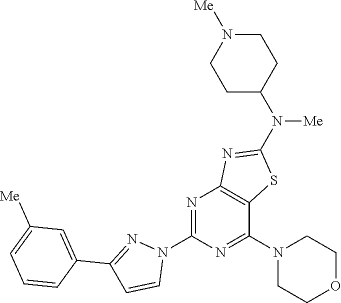 [SMILES: Cc1cccc(-c2ccn(-c3nc(N4CCOCC4)c4sc(N(C)C5CCN(C)CC5)nc4n3)n2)c1] |
| 209 | 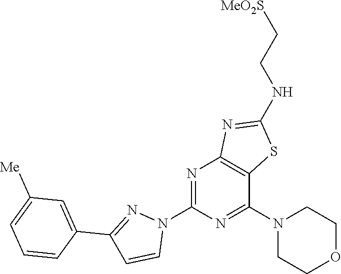 [SMILES: Cc1cccc(-c2ccn(-c3nc(N4CCOCC4)c4sc(NCCS(C)(=O)=O)nc4n3)n2)c1] |
| 210 | 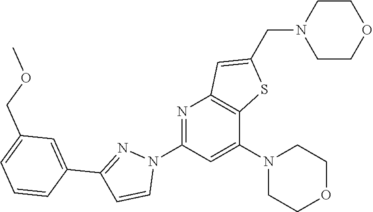 [SMILES: COCc1cccc(-c2ccn(-c3cc(N4CCOCC4)c4sc(CN5CCOCC5)cc4n3)n2)c1] |
| 211 | 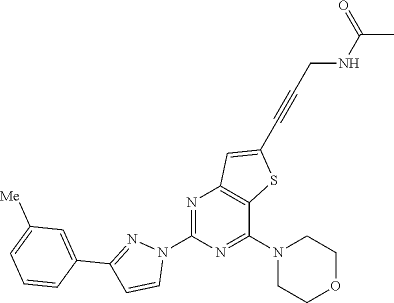 [SMILES: CC(=O)NCC#Cc1cc2nc(-n3ccc(-c4cccc(C)c4)n3)nc(N3CCOCC3)c2s1] |
| 212 & 213 | 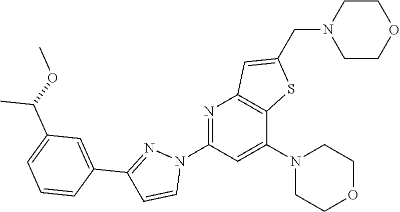 [SMILES: CO[C@@H](C)c1cccc(-c2ccn(-c3cc(N4CCOCC4)c4sc(CN5CCOCC5)cc4n3)n2)c1] |
| | 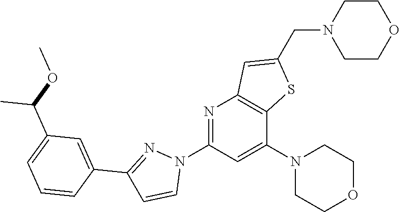 [SMILES: CO[C@H](C)c1cccc(-c2ccn(-c3cc(N4CCOCC4)c4sc(CN5CCOCC5)cc4n3)n2)c1] |
| 214 & 215 | 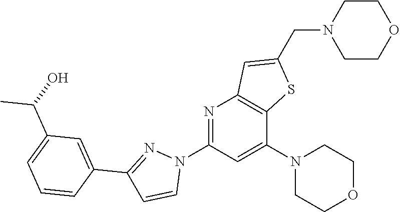 [SMILES: C[C@H](O)c1cccc(-c2ccn(-c3cc(N4CCOCC4)c4sc(CN5CCOCC5)cc4n3)n2)c1] |
| | 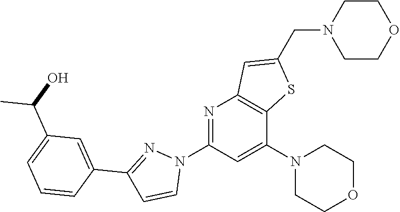 [SMILES: C[C@@H](O)c1cccc(-c2ccn(-c3cc(N4CCOCC4)c4sc(CN5CCOCC5)cc4n3)n2)c1] |
| 216 | 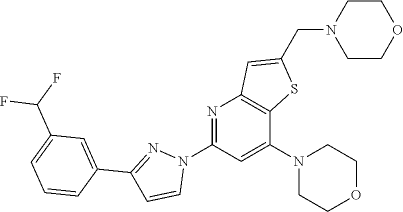 [SMILES: FC(F)c1cccc(-c2ccn(-c3cc(N4CCOCC4)c4sc(CN5CCOCC5)cc4n3)n2)c1] |
| 217 | 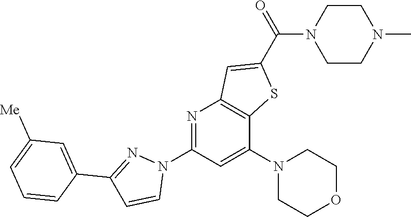 [SMILES: Cc1cccc(-c2ccn(-c3cc(N4CCOCC4)c4sc(C(=O)N5CCN(C)CC5)cc4n3)n2)c1] |
| 218 | 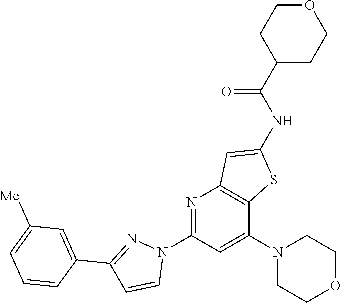 [SMILES: Cc1cccc(-c2ccn(-c3cc(N4CCOCC4)c4sc(NC(=O)C5CCOCC5)cc4n3)n2)c1] |
| 263 | 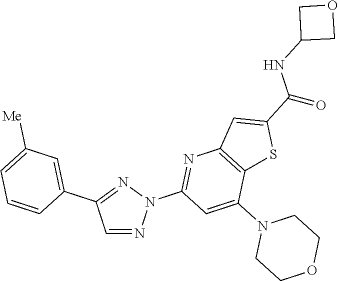 [SMILES: Cc1cccc(-c2cnn(-c3cc(N4CCOCC4)c4sc(C(=O)NC5COC5)cc4n3)n2)c1] |
| 264 | 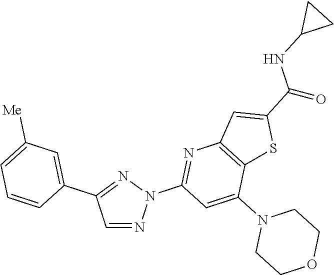 [SMILES: Cc1cccc(-c2cnn(-c3cc(N4CCOCC4)c4sc(C(=O)NC5CC5)cc4n3)n2)c1] |
| 265 | 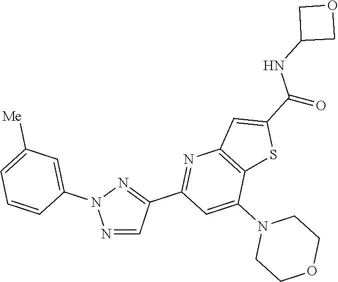 [SMILES: Cc1cccc(-n2ncc(-c3cc(N4CCOCC4)c4sc(C(=O)NC5COC5)cc4n3)n2)c1] |
| 266 | 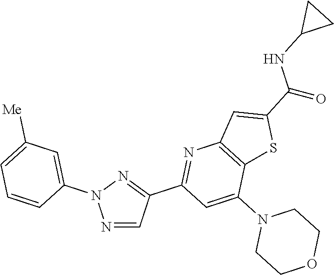 [SMILES: Cc1cccc(-n2ncc(-c3cc(N4CCOCC4)c4sc(C(=O)NC5CC5)cc4n3)n2)c1] |
| 268 | 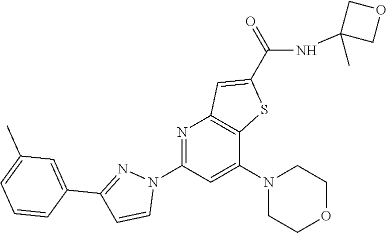 [SMILES: Cc1cccc(-c2ccn(-c3cc(N4CCOCC4)c4sc(C(=O)NC5(C)COC5)cc4n3)n2)c1] |
| 269 | 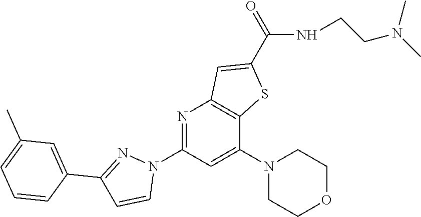 [SMILES: Cc1cccc(-c2ccn(-c3cc(N4CCOCC4)c4sc(C(=O)NCCN(C)C)cc4n3)n2)c1] |

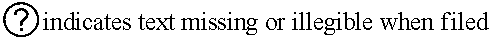

TABLE 2

| Ex. No. | Structure |
|---|---|
| 87 | 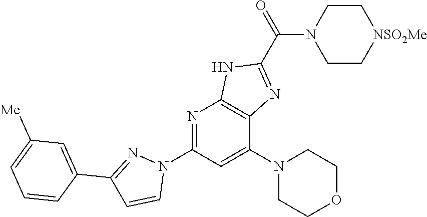 |
| 88 | 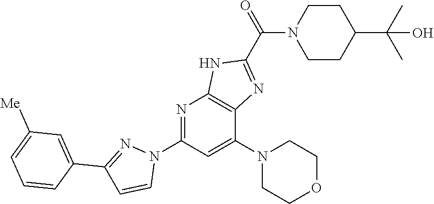 [SMILES: Cc1cccc(-c2ccn(-c3cc(N4CCOCC4)c4nc(C(=O)N5CCC(C(C)(C)O)CC5)[nH]c4n3)n2)c1] |
| 89 | 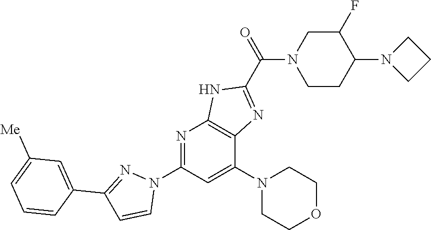 [SMILES: Cc1cccc(-c2ccn(-c3cc(N4CCOCC4)c4nc(C(=O)N5CCC(N6CCC6)C(F)C5)[nH]c4n3)n2)c1] |
| 90 | 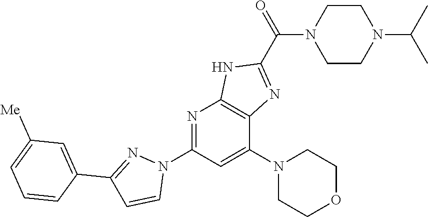 [SMILES: Cc1cccc(-c2ccn(-c3cc(N4CCOCC4)c4nc(C(=O)N5CCN(C(C)C)CC5)[nH]c4n3)n2)c1] |
| 91 | 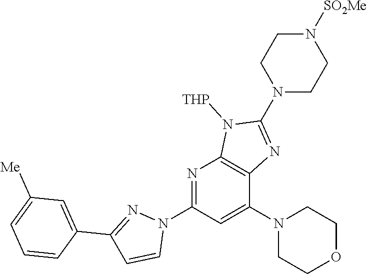 |
| 92 | 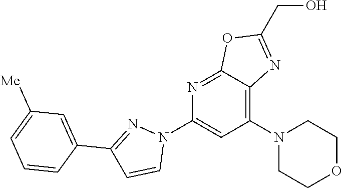 [SMILES: Cc1cccc(-c2ccn(-c3cc(N4CCOCC4)c4nc(CO)oc4n3)n2)c1] |
| 93 | 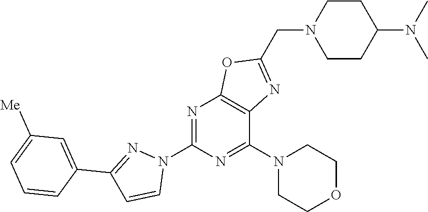 [SMILES: Cc1cccc(-c2ccn(-c3nc(N4CCOCC4)c4nc(CN5CCC(N(C)C)CC5)oc4n3)n2)c1] |
| 94 | 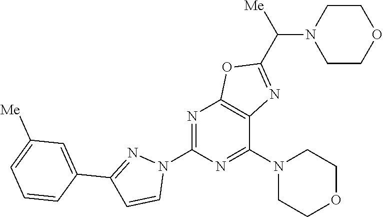 [SMILES: Cc1cccc(-c2ccn(-c3nc(N4CCOCC4)c4nc(C(C)N5CCOCC5)oc4n3)n2)c1] |
| 95 | 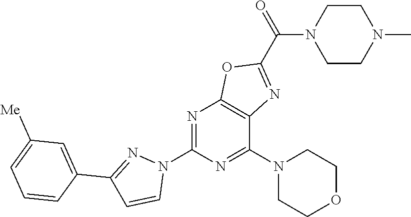 [SMILES: Cc1cccc(-c2ccn(-c3nc(N4CCOCC4)c4nc(C(=O)N5CCN(C)CC5)oc4n3)n2)c1] |
| 96 | 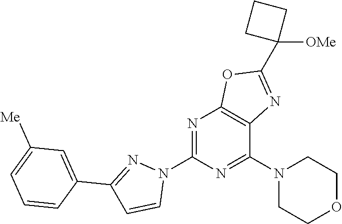 [SMILES: COC1(c2nc3c(N4CCOCC4)nc(-n4ccc(-c5cccc(C)c5)n4)nc3o2)CCC1] |
| 97 | 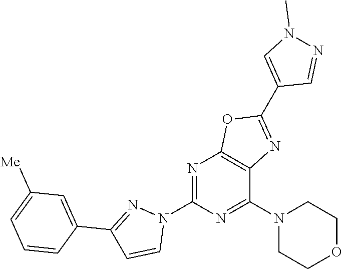 [SMILES: Cc1cccc(-c2ccn(-c3nc(N4CCOCC4)c4nc(-c5cnn(C)c5)oc4n3)n2)c1] |
| 98 | 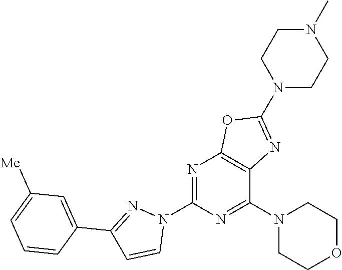 [SMILES: Cc1cccc(-c2ccn(-c3nc(N4CCOCC4)c4nc(N5CCN(C)CC5)oc4n3)n2)c1] |
| 99 | 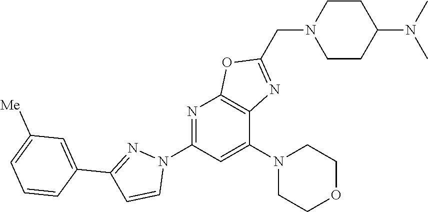 [SMILES: Cc1cccc(-c2ccn(-c3cc(N4CCOCC4)c4nc(CN5CCC(N(C)C)CC5)oc4n3)n2)c1] |
| 100 | 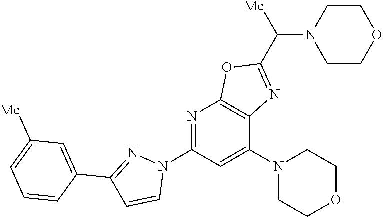 [SMILES: Cc1cccc(-c2ccn(-c3cc(N4CCOCC4)c4nc(C(C)N5CCOCC5)oc4n3)n2)c1] |
| 101 | 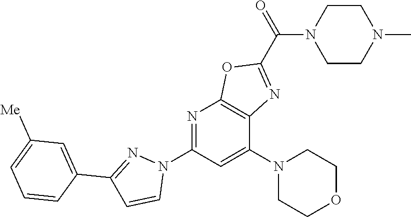 [SMILES: Cc1cccc(-c2ccn(-c3cc(N4CCOCC4)c4nc(C(=O)N5CCN(C)CC5)oc4n3)n2)c1] |
| 102 | 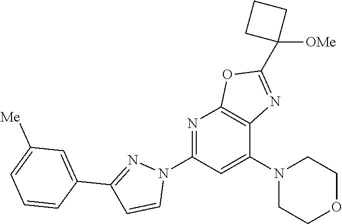 [SMILES: COC1(c2nc3c(N4CCOCC4)cc(-n4ccc(-c5cccc(C)c5)n4)nc3o2)CCC1] |
| 103 | 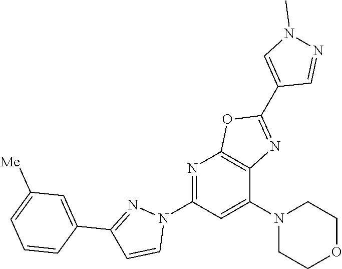 [SMILES: Cc1cccc(-c2ccn(-c3cc(N4CCOCC4)c4nc(-c5cnn(C)c5)oc4n3)n2)c1] |
| 104 | 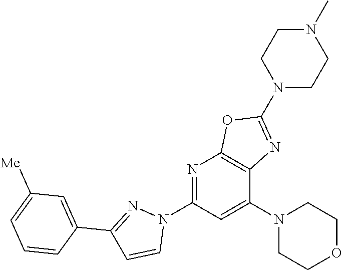 [SMILES: Cc1cccc(-c2ccn(-c3cc(N4CCOCC4)c4nc(N5CCN(C)CC5)oc4n3)n2)c1] |
| 105 | 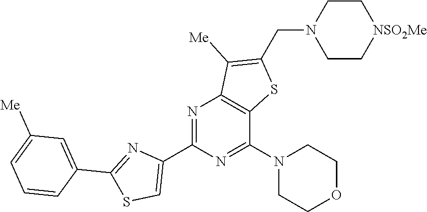 |
| 106 | 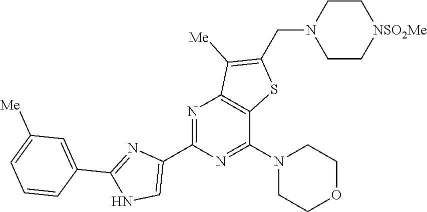 |
| 107 | 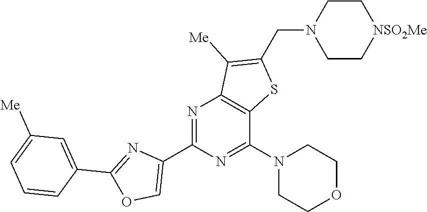 |
| 108 | 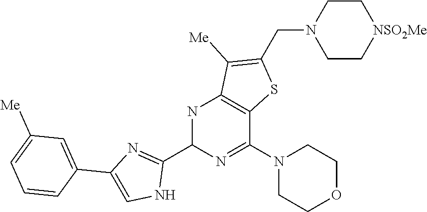 |
| 109 | 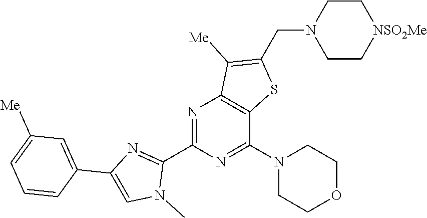 |
| 110 | 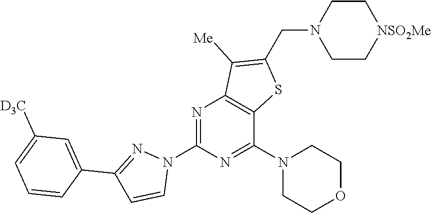 |
| 111 | 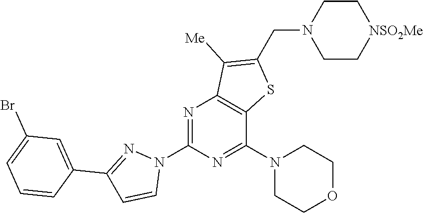 |
| 112 | 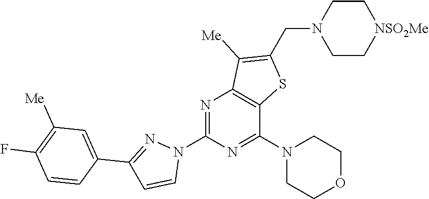 |
| 113 | 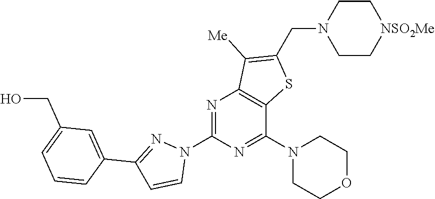 |
| 114 | 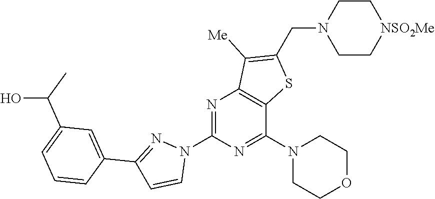 |
| 115 | 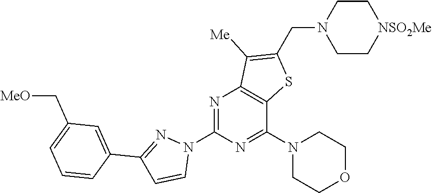 |
| 116 | 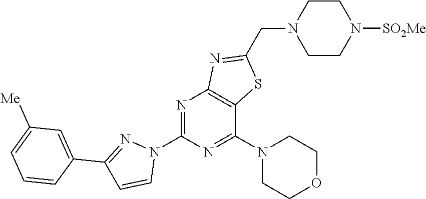 [SMILES: Cc1cccc(-c2ccn(-c3nc(N4CCOCC4)c4sc(CN5CCN(S(C)(=O)=O)CC5)nc4n3)n2)c1] |
| 117 | 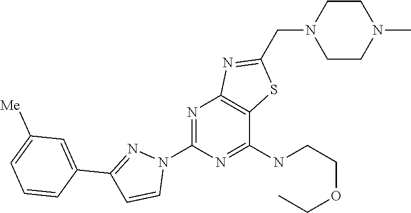 [SMILES: CCOCCNc1nc(-n2ccc(-c3cccc(C)c3)n2)nc2nc(CN3CCN(C)CC3)sc12] |
| 118 | 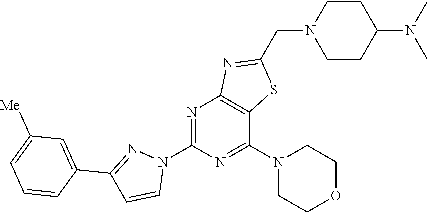 [SMILES: Cc1cccc(-c2ccn(-c3nc(N4CCOCC4)c4sc(CN5CCC(N(C)C)CC5)nc4n3)n2)c1] |
| 119 | 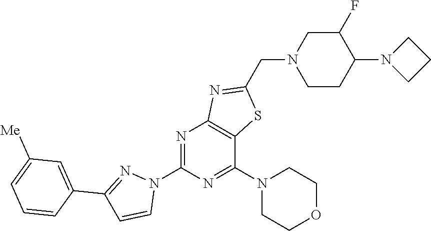 [SMILES: Cc1cccc(-c2ccn(-c3nc(N4CCOCC4)c4sc(CN5CCC(N6CCC6)C(F)C5)nc4n3)n2)c1] |
| 120 | 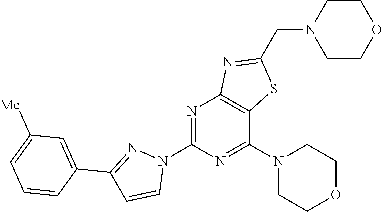 [SMILES: Cc1cccc(-c2ccn(-c3nc(N4CCOCC4)c4sc(CN5CCOCC5)nc4n3)n2)c1] |
| 121 | 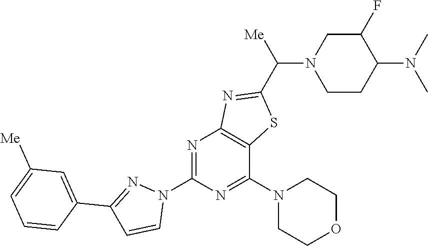 [SMILES: Cc1cccc(-c2ccn(-c3nc(N4CCOCC4)c4sc(C(C)N5CCC(N(C)C)C(F)C5)nc4n3)n2)c1] |
| 122 | 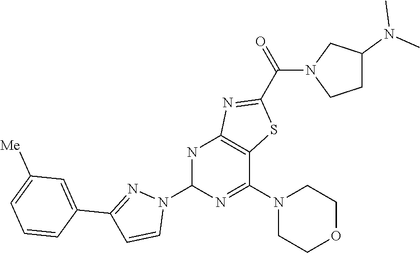 [SMILES: Cc1cccc(-c2ccn(C3N=C(N4CCOCC4)c4sc(C(=O)N5CCC(N(C)C)C5)nc4N3)n2)c1] |
| 123 | 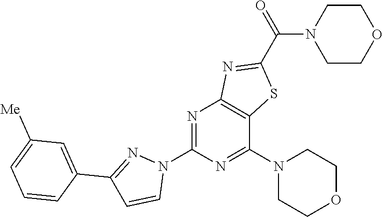 [SMILES: Cc1cccc(-c2ccn(-c3nc(N4CCOCC4)c4sc(C(=O)N5CCOCC5)nc4n3)n2)c1] |
| 124 | 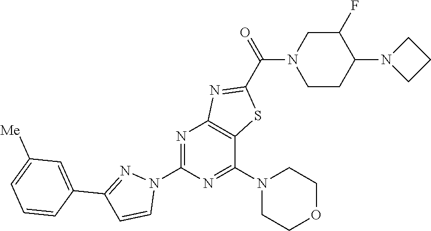 [SMILES: Cc1cccc(-c2ccn(-c3nc(N4CCOCC4)c4sc(C(=O)N5CCC(N6CCC6)C(F)C5)nc4n3)n2)c1] |
| 125 | 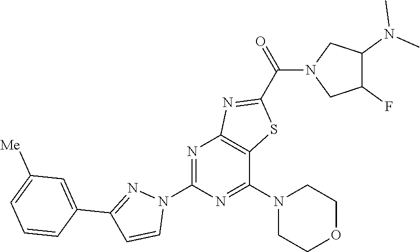 [SMILES: Cc1cccc(-c2ccn(-c3nc(N4CCOCC4)c4sc(C(=O)N5CC(F)C(N(C)C)C5)nc4n3)n2)c1] |
| 126 | 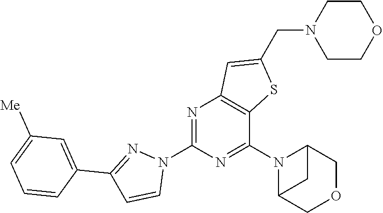 [SMILES: Cc1cccc(-c2ccn(-c3nc(N4C5COCC4C5)c4sc(CN5CCOCC5)cc4n3)n2)c1] |
| 127 | 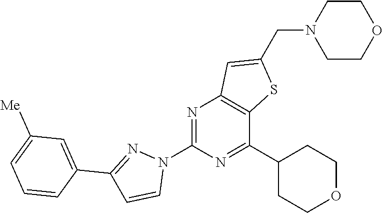 [SMILES: Cc1cccc(-c2ccn(-c3nc(C4CCOCC4)c4sc(CN5CCOCC5)cc4n3)n2)c1] |
| 128 | 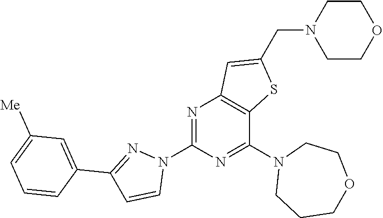 [SMILES: Cc1cccc(-c2ccn(-c3nc(N4CCCOCC4)c4sc(CN5CCOCC5)cc4n3)n2)c1] |
| 130 | 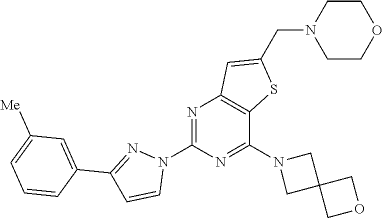 [SMILES: Cc1cccc(-c2ccn(-c3nc(N4CC5(COC5)C4)c4sc(CN5CCOCC5)cc4n3)n2)c1] |
| 131 | 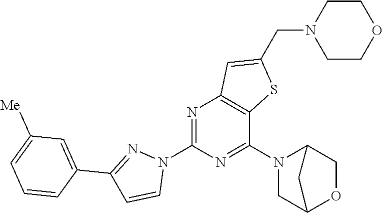 [SMILES: Cc1cccc(-c2ccn(-c3nc(N4CC5CC4CO5)c4sc(CN5CCOCC5)cc4n3)n2)c1] |
| 133 | 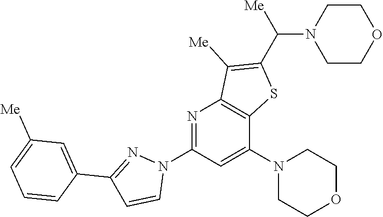 [SMILES: Cc1cccc(-c2ccn(-c3cc(N4CCOCC4)c4sc(C(C)N5CCOCC5)c(C)c4n3)n2)c1] |
| 134 | 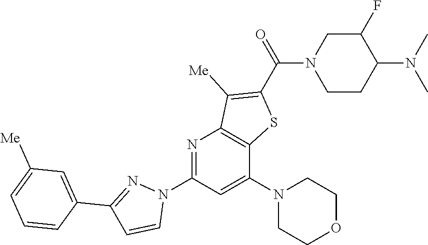 [SMILES: Cc1cccc(-c2ccn(-c3cc(N4CCOCC4)c4sc(C(=O)N5CCC(N(C)C)C(F)C5)c(C)c4n3)n2)c1] |
| 135 | 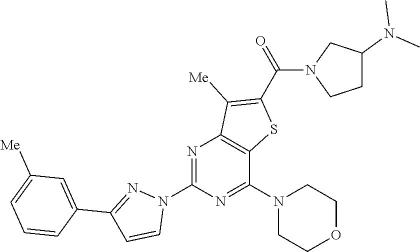 [SMILES: Cc1cccc(-c2ccn(-c3nc(N4CCOCC4)c4sc(C(=O)N5CCC(N(C)C)C5)c(C)c4n3)n2)c1] |
| 136 | 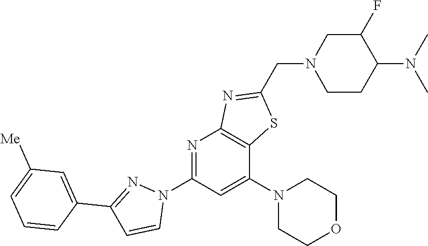 [SMILES: Cc1cccc(-c2ccn(-c3cc(N4CCOCC4)c4sc(CN5CCC(N(C)C)C(F)C5)nc4n3)n2)c1] |
| 138 | 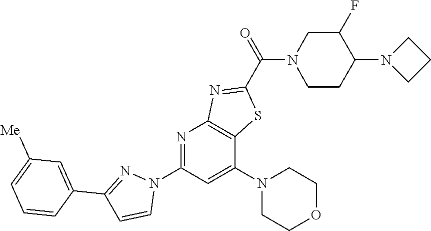 [SMILES: Cc1cccc(-c2ccn(-c3cc(N4CCOCC4)c4sc(C(=O)N5CCC(N6CCC6)C(F)C5)nc4n3)n2)c1] |
| 139 | 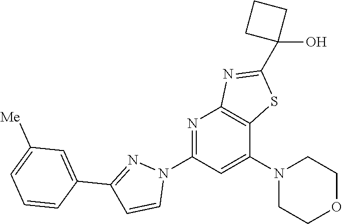 [SMILES: Cc1cccc(-c2ccn(-c3cc(N4CCOCC4)c4sc(C5(O)CCC5)nc4n3)n2)c1] |
| 140 | 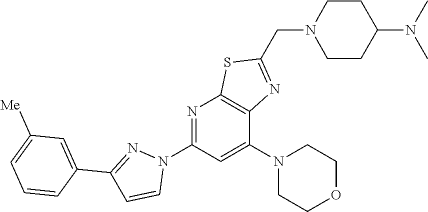 [SMILES: Cc1cccc(-c2ccn(-c3cc(N4CCOCC4)c4nc(CN5CCC(N(C)C)CC5)sc4n3)n2)c1] |
| 141 | 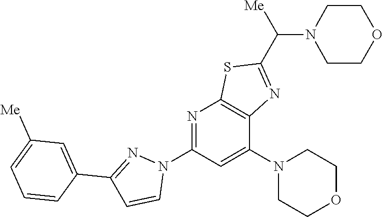 [SMILES: Cc1cccc(-c2ccn(-c3cc(N4CCOCC4)c4nc(C(C)N5CCOCC5)sc4n3)n2)c1] |
| 142 | 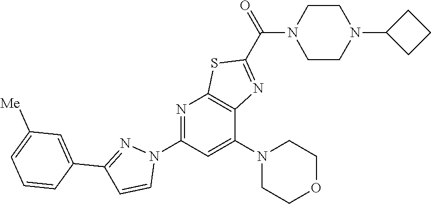 [SMILES: Cc1cccc(-c2ccn(-c3cc(N4CCOCC4)c4nc(C(=O)N5CCN(C6CCC6)CC5)sc4n3)n2)c1] |
| 144 | 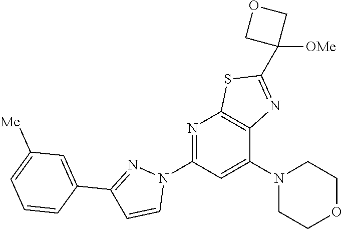 [SMILES: COC1(c2nc3c(N4CCOCC4)cc(-n4ccc(-c5cccc(C)c5)n4)nc3s2)COC1] |
| 145 | 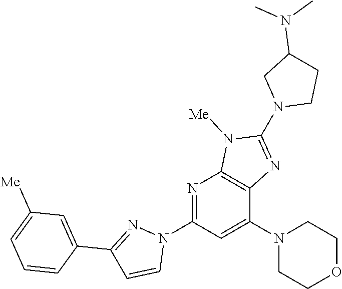 [SMILES: Cc1cccc(-c2ccn(-c3cc(N4CCOCC4)c4nc(N5CCC(N(C)C)C5)n(C)c4n3)n2)c1] |
| 146 | 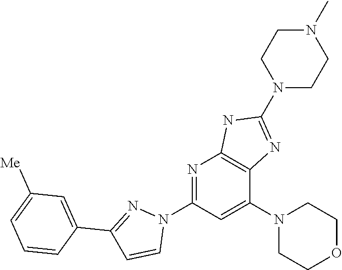 [SMILES: Cc1cccc(-c2ccn(-c3cc(N4CCOCC4)c4nc(N5CCN(C)CC5)[nH]c4n3)n2)c1] |
| 147 | 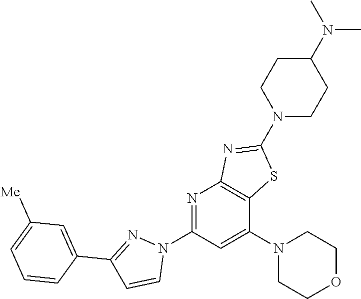 [SMILES: Cc1cccc(-c2ccn(-c3cc(N4CCOCC4)c4sc(N5CCC(N(C)C)CC5)nc4n3)n2)c1] |
| 148 | 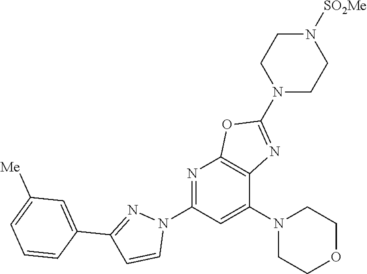 [SMILES: Cc1cccc(-c2ccn(-c3cc(N4CCOCC4)c4nc(N5CCN(S(C)(=O)=O)CC5)oc4n3)n2)c1] |
| 149 | 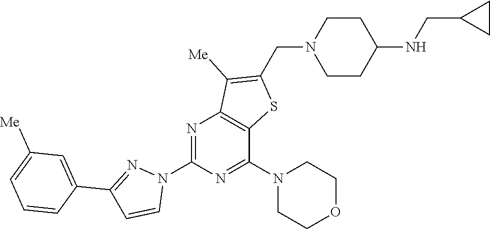 [SMILES: Cc1cccc(-c2ccn(-c3nc(N4CCOCC4)c4sc(CN5CCC(NCC6CC6)CC5)c(C)c4n3)n2)c1] |
| 150 | 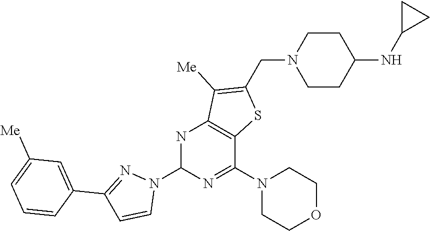 [SMILES: Cc1cccc(-c2ccn(C3N=C(N4CCOCC4)c4sc(CN5CCC(NC6CC6)CC5)c(C)c4N3)n2)c1] |
| 151 | 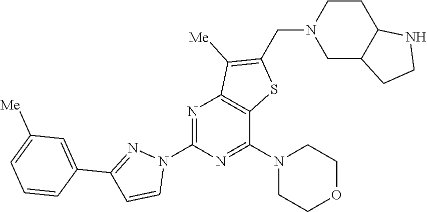 [SMILES: Cc1cccc(-c2ccn(-c3nc(N4CCOCC4)c4sc(CN5CCC6NCCC6C5)c(C)c4n3)n2)c1] |
| 152 | 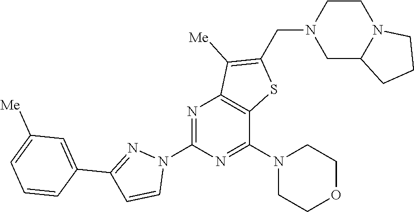 [SMILES: Cc1cccc(-c2ccn(-c3nc(N4CCOCC4)c4sc(CN5CCN6CCCC6C5)c(C)c4n3)n2)c1] |
| 153 | 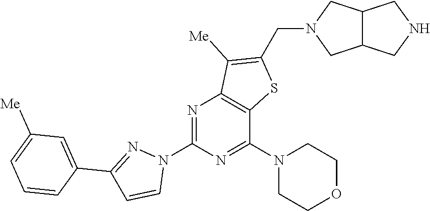 [SMILES: Cc1cccc(-c2ccn(-c3nc(N4CCOCC4)c4sc(CN5CC6CNCC6C5)c(C)c4n3)n2)c1] |
| 154 | 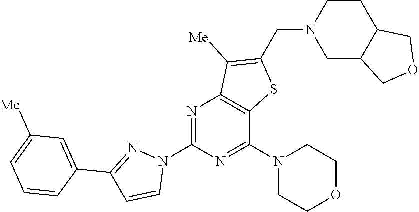 [SMILES: Cc1cccc(-c2ccn(-c3nc(N4CCOCC4)c4sc(CN5CCC6COCC6C5)c(C)c4n3)n2)c1] |
| 155 | 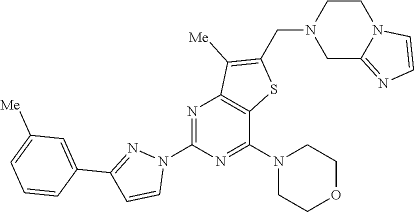 [SMILES: Cc1cccc(-c2ccn(-c3nc(N4CCOCC4)c4sc(CN5CCn6ccnc6C5)c(C)c4n3)n2)c1] |
| 156 | 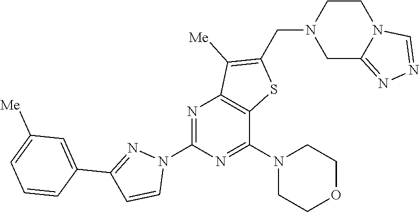 [SMILES: Cc1cccc(-c2ccn(-c3nc(N4CCOCC4)c4sc(CN5CCn6cnnc6C5)c(C)c4n3)n2)c1] |
| 157 | 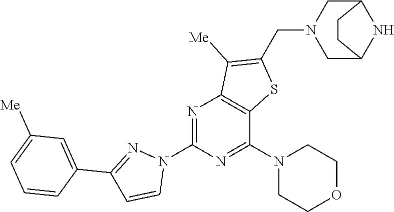 [SMILES: Cc1cccc(-c2ccn(-c3nc(N4CCOCC4)c4sc(CN5CC6CCC(C5)N6)c(C)c4n3)n2)c1] |
| 158 | 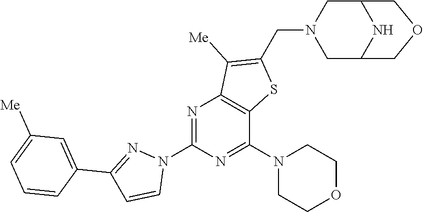 [SMILES: Cc1cccc(-c2ccn(-c3nc(N4CCOCC4)c4sc(CN5CC6COCC(C5)N6)c(C)c4n3)n2)c1] |
| 159 | 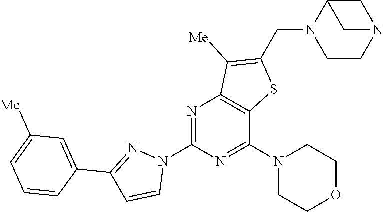 [SMILES: Cc1cccc(-c2ccn(-c3nc(N4CCOCC4)c4sc(CN5CCN6CC5C6)c(C)c4n3)n2)c1] |
| 160 | 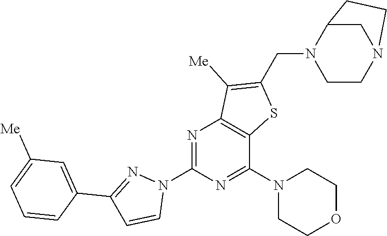 [SMILES: Cc1cccc(-c2ccn(-c3nc(N4CCOCC4)c4sc(CN5CCN6CCC5C6)c(C)c4n3)n2)c1] |
| 161 | 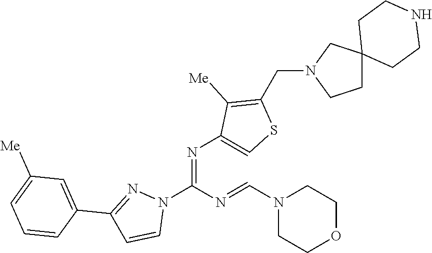 [SMILES: Cc1cccc(-c2ccn(C(/N=C/N3CCOCC3)=N/c3csc(CN4CCC5(CCNCC5)C4)c3C)n2)c1] |
| 162 | 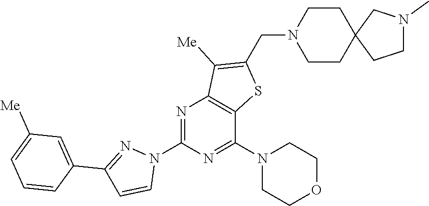 [SMILES: Cc1cccc(-c2ccn(-c3nc(N4CCOCC4)c4sc(CN5CCC6(CCN(C)C6)CC5)c(C)c4n3)n2)c1] |
| 163 | 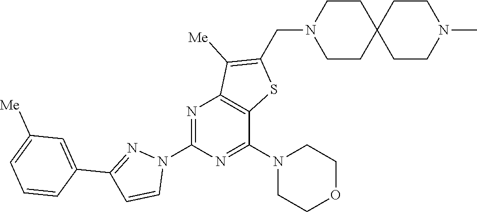 [SMILES: Cc1cccc(-c2ccn(-c3nc(N4CCOCC4)c4sc(CN5CCC6(CCN(C)CC6)CC5)c(C)c4n3)n2)c1] |
| 164 | 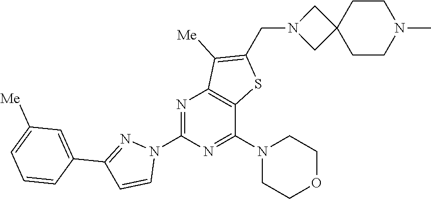 [SMILES: Cc1cccc(-c2ccn(-c3nc(N4CCOCC4)c4sc(CN5CC6(CCN(C)CC6)C5)c(C)c4n3)n2)c1] |
| 219 | 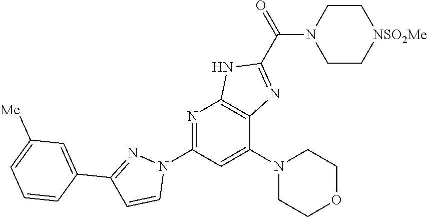 |
| 220 | 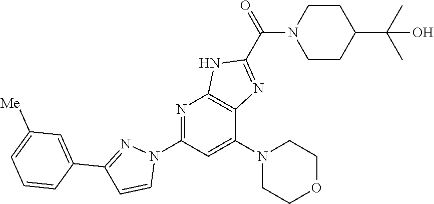 [SMILES: Cc1cccc(-c2ccn(-c3cc(N4CCOCC4)c4nc(C(=O)N5CCC(C(C)(C)O)CC5)[nH]c4n3)n2)c1] |
| 221 | 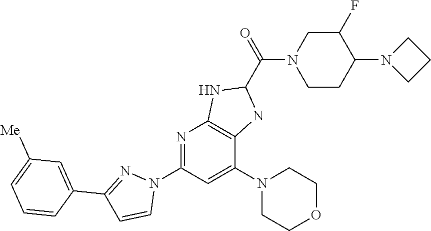 [SMILES: Cc1cccc(-c2ccn(-c3cc(N4CCOCC4)c4c(n3)NC(C(=O)N3CCC(N5CCC5)C(F)C3)N4)n2)c1] |
| 222 | 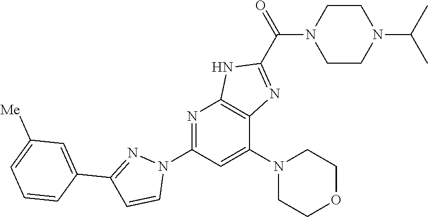 [SMILES: Cc1cccc(-c2ccn(-c3cc(N4CCOCC4)c4nc(C(=O)N5CCN(C(C)C)CC5)[nH]c4n3)n2)c1] |
| 223 | 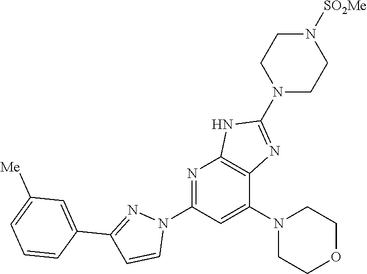 [SMILES: Cc1cccc(-c2ccn(-c3cc(N4CCOCC4)c4nc(N5CCN(S(C)(=O)=O)CC5)[nH]c4n3)n2)c1] |
| 224 | 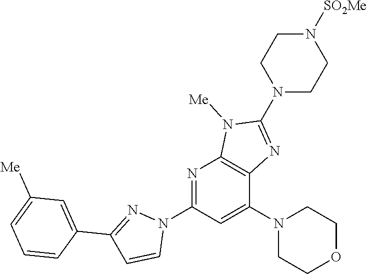 [SMILES: Cc1cccc(-c2ccn(-c3cc(N4CCOCC4)c4nc(N5CCN(S(C)(=O)=O)CC5)n(C)c4n3)n2)c1] |
| 225 | 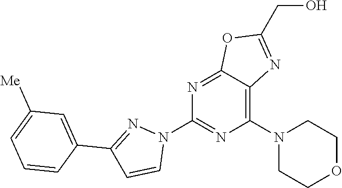 [SMILES: Cc1cccc(-c2ccn(-c3nc(N4CCOCC4)c4nc(CO)oc4n3)n2)c1] |
| 226 | 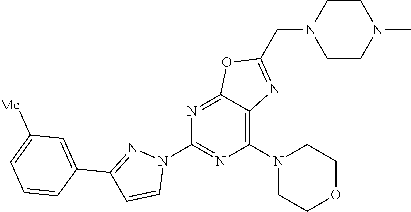 [SMILES: Cc1cccc(-c2ccn(-c3nc(N4CCOCC4)c4nc(CN5CCN(C)CC5)oc4n3)n2)c1] |
| 227 | 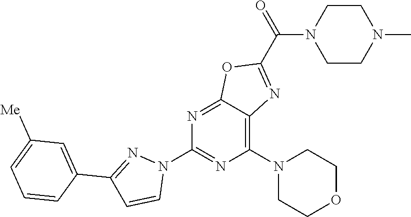 [SMILES: Cc1cccc(-c2ccn(-c3nc(N4CCOCC4)c4nc(C(=O)N5CCN(C)CC5)oc4n3)n2)c1] |
| 228 | 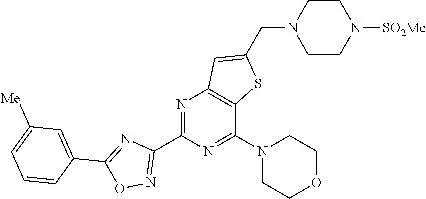 [SMILES: Cc1cccc(-c2nc(-c3nc(N4CCOCC4)c4sc(CN5CCN(S(C)(=O)=O)CC5)cc4n3)no2)c1] |
| 229 | 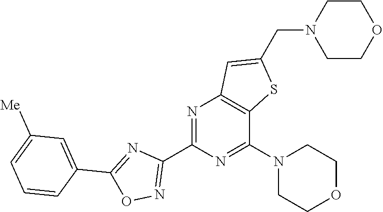 [SMILES: Cc1cccc(-c2nc(-c3nc(N4CCOCC4)c4sc(CN5CCOCC5)cc4n3)no2)c1] |
| 230 | 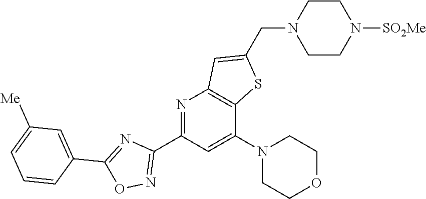 [SMILES: Cc1cccc(-c2nc(-c3cc(N4CCOCC4)c4sc(CN5CCN(S(C)(=O)=O)CC5)cc4n3)no2)c1] |
| 231 | 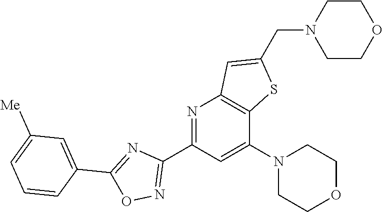 [SMILES: Cc1cccc(-c2nc(-c3cc(N4CCOCC4)c4sc(CN5CCOCC5)cc4n3)no2)c1] |
| 232 | 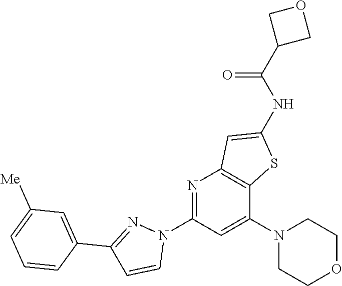 [SMILES: Cc1cccc(-c2ccn(-c3cc(N4CCOCC4)c4sc(NC(=O)C5COC5)cc4n3)n2)c1] |
| 233 | 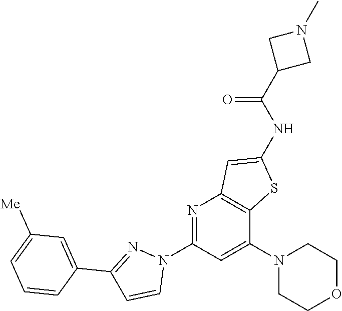 [SMILES: Cc1cccc(-c2ccn(-c3cc(N4CCOCC4)c4sc(NC(=O)C5CN(C)C5)cc4n3)n2)c1] |
| 234 | 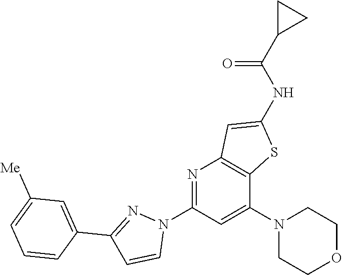 [SMILES: Cc1cccc(-c2ccn(-c3cc(N4CCOCC4)c4sc(NC(=O)C5CC5)cc4n3)n2)c1] |
| 235 | 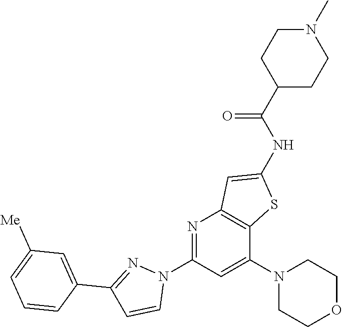 [SMILES: Cc1cccc(-c2ccn(-c3cc(N4CCOCC4)c4sc(NC(=O)C5CCN(C)CC5)cc4n3)n2)c1] |
| 236 | 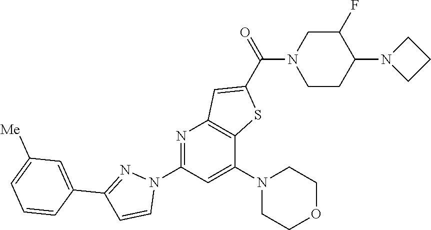 [SMILES: Cc1cccc(-c2ccn(-c3cc(N4CCOCC4)c4sc(C(=O)N5CCC(N6CCC6)C(F)C5)cc4n3)n2)c1] |
| 237 | 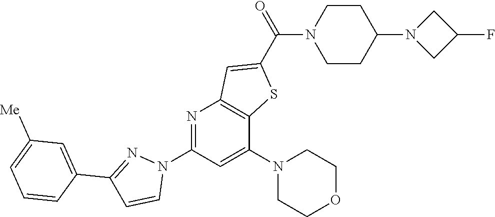 [SMILES: Cc1cccc(-c2ccn(-c3cc(N4CCOCC4)c4sc(C(=O)N5CCC(N6CC(F)C6)CC5)cc4n3)n2)c1] |
| 238 | 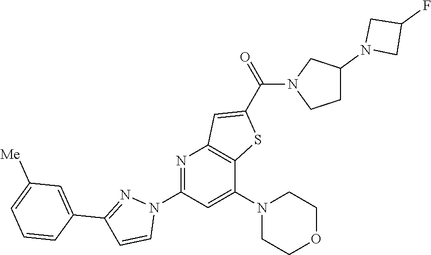 [SMILES: Cc1cccc(-c2ccn(-c3cc(N4CCOCC4)c4sc(C(=O)N5CCC(N6CC(F)C6)C5)cc4n3)n2)c1] |
| 239 | 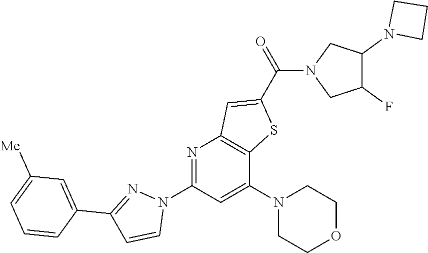 [SMILES: Cc1cccc(-c2ccn(-c3cc(N4CCOCC4)c4sc(C(=O)N5CC(F)C(N6CCC6)C5)cc4n3)n2)c1] |
| 240 | 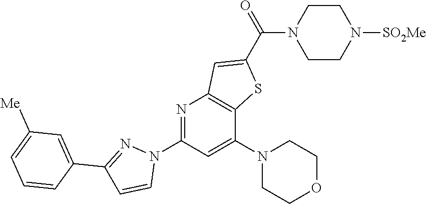 [SMILES: Cc1cccc(-c2ccn(-c3cc(N4CCOCC4)c4sc(C(=O)N5CCN(S(C)(=O)=O)CC5)cc4n3)n2)c1] |
| 241 | 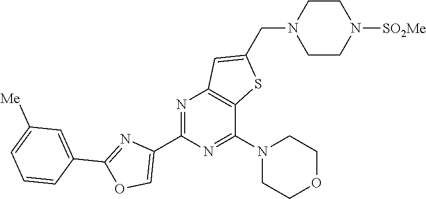 [SMILES: Cc1cccc(-c2nc(-c3nc(N4CCOCC4)c4sc(CN5CCN(S(C)(=O)=O)CC5)cc4n3)co2)c1] |
| 242 | 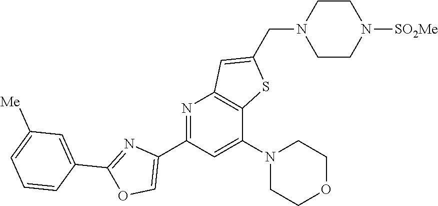 [SMILES: Cc1cccc(-c2nc(-c3cc(N4CCOCC4)c4sc(CN5CCN(S(C)(=O)=O)CC5)cc4n3)co2)c1] |
| 243 | 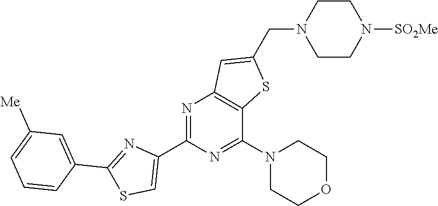 [SMILES: Cc1cccc(-c2nc(-c3nc(N4CCOCC4)c4sc(CN5CCN(S(C)(=O)=O)CC5)cc4n3)cs2)c1] |
| 244 | 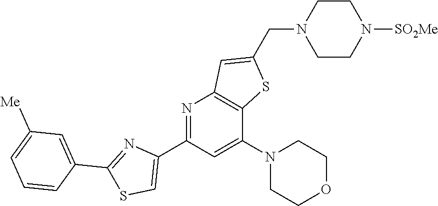 [SMILES: Cc1cccc(-c2nc(-c3cc(N4CCOCC4)c4sc(CN5CCN(S(C)(=O)=O)CC5)cc4n3)cs2)c1] |
| 245 | 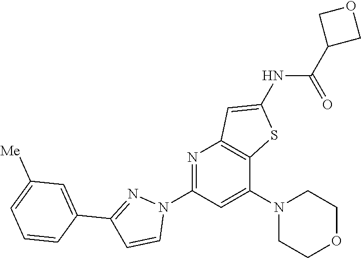 [SMILES: Cc1cccc(-c2ccn(-c3cc(N4CCOCC4)c4sc(NC(=O)C5COC5)cc4n3)n2)c1] |
| 246 | 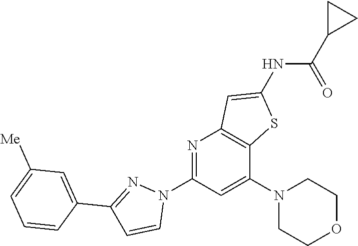 [SMILES: Cc1cccc(-c2ccn(-c3cc(N4CCOCC4)c4sc(NC(=O)C5CC5)cc4n3)n2)c1] |
| 247 | 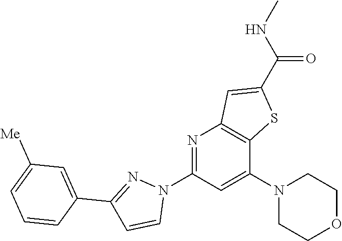 [SMILES: CNC(=O)c1cc2nc(-n3ccc(-c4cccc(C)c4)n3)cc(N3CCOCC3)c2s1] |
| 248 | 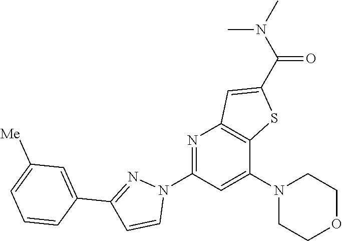 [SMILES: Cc1cccc(-c2ccn(-c3cc(N4CCOCC4)c4sc(C(=O)N(C)C)cc4n3)n2)c1] |
| 249 | 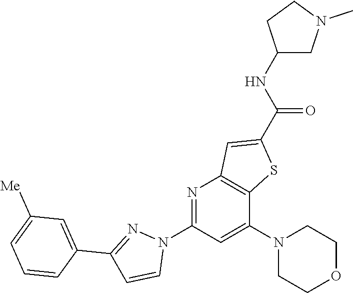 [SMILES: Cc1cccc(-c2ccn(-c3cc(N4CCOCC4)c4sc(C(=O)NC5CCN(C)C5)cc4n3)n2)c1] |
| 250 | 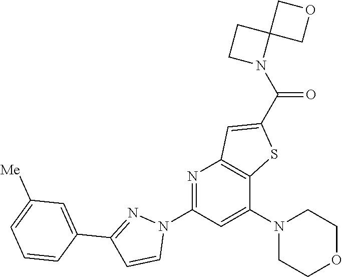 [SMILES: Cc1cccc(-c2ccn(-c3cc(N4CCOCC4)c4sc(C(=O)N5CCC56COC6)cc4n3)n2)c1] |
| 251 | 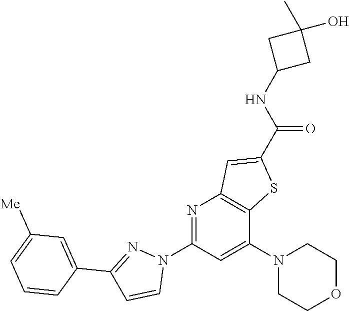 [SMILES: Cc1cccc(-c2ccn(-c3cc(N4CCOCC4)c4sc(C(=O)NC5CC(C)(O)C5)cc4n3)n2)c1] |
| 252 | 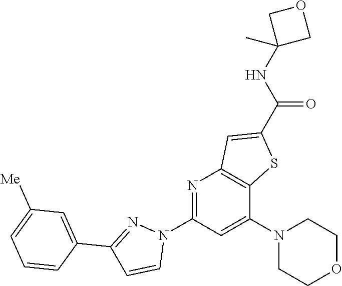 [SMILES: Cc1cccc(-c2ccn(-c3cc(N4CCOCC4)c4sc(C(=O)NC5(C)COC5)cc4n3)n2)c1] |
| 253 | 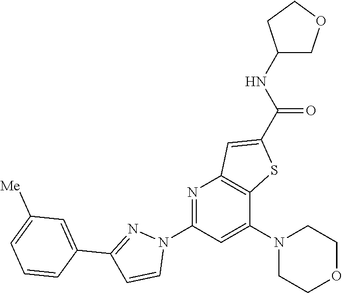 [SMILES: Cc1cccc(-c2ccn(-c3cc(N4CCOCC4)c4sc(C(=O)NC5CCOC5)cc4n3)n2)c1] |
| 254 | 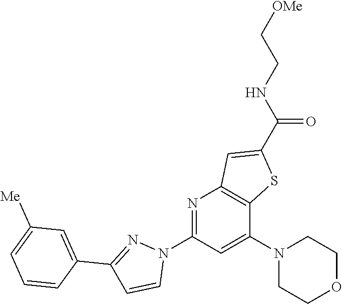 [SMILES: COCCNC(=O)c1cc2nc(-n3ccc(-c4cccc(C)c4)n3)cc(N3CCOCC3)c2s1] |
| 255 | 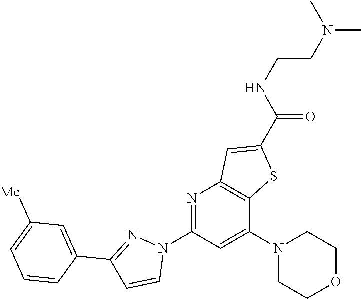 [SMILES: Cc1cccc(-c2ccn(-c3cc(N4CCOCC4)c4sc(C(=O)NCCN(C)C)cc4n3)n2)c1] |
| 256 | 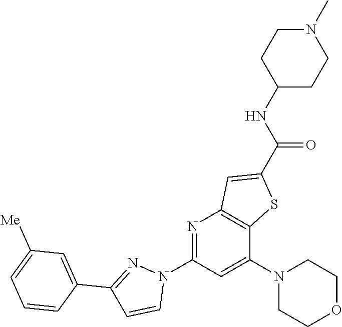 [SMILES: Cc1cccc(-c2ccn(-c3cc(N4CCOCC4)c4sc(C(=O)NC5CCN(C)CC5)cc4n3)n2)c1] |
| 257 | 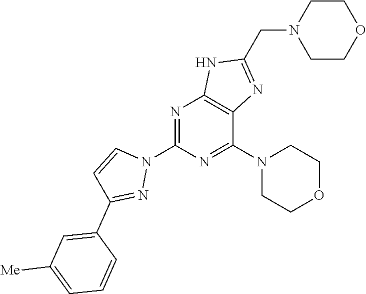 [SMILES: Cc1cccc(-c2ccn(-c3nc(N4CCOCC4)c4nc(CN5CCOCC5)[nH]c4n3)n2)c1] |
| 258 | 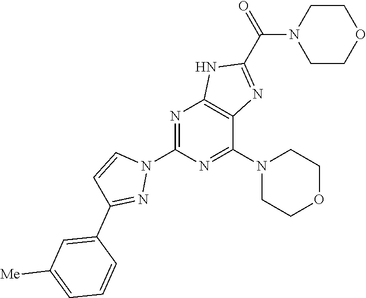 [SMILES: Cc1cccc(-c2ccn(-c3nc(N4CCOCC4)c4nc(C(=O)N5CCOCC5)[nH]c4n3)n2)c1] |
| 259 | 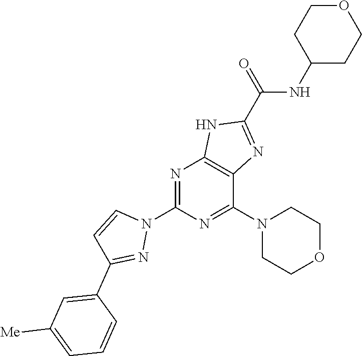 [SMILES: Cc1cccc(-c2ccn(-c3nc(N4CCOCC4)c4nc(C(=O)NC5CCOCC5)[nH]c4n3)n2)c1] |
| 260 | 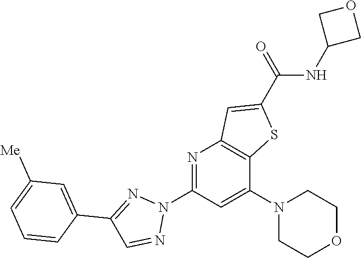 [SMILES: Cc1cccc(-c2cnn(-c3cc(N4CCOCC4)c4sc(C(=O)NC5COC5)cc4n3)n2)c1] |
| 261 | 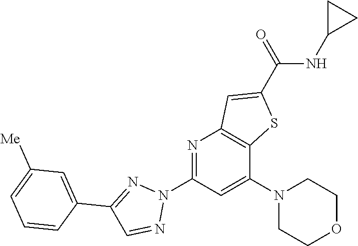 [SMILES: Cc1cccc(-c2cnn(-c3cc(N4CCOCC4)c4sc(C(=O)NC5CC5)cc4n3)n2)c1] |
| 262 | 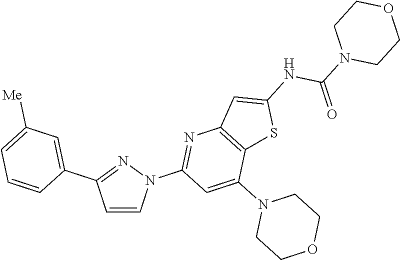 [SMILES: Cc1cccc(-c2ccn(-c3cc(N4CCOCC4)c4sc(NC(=O)N5CCOCC5)cc4n3)n2)c1] |
| 267 | 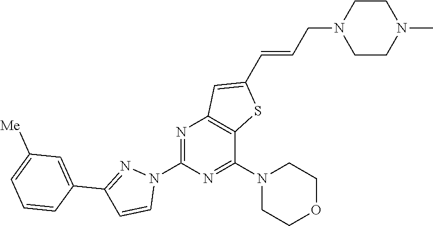 [SMILES: Cc1cccc(-c2ccn(-c3nc(N4CCOCC4)c4sc(/C=C/CN5CCN(C)CC5)cc4n3)n2)c1] |

General Schemes

The compounds of Examples 1-7 are prepared according to the following scheme

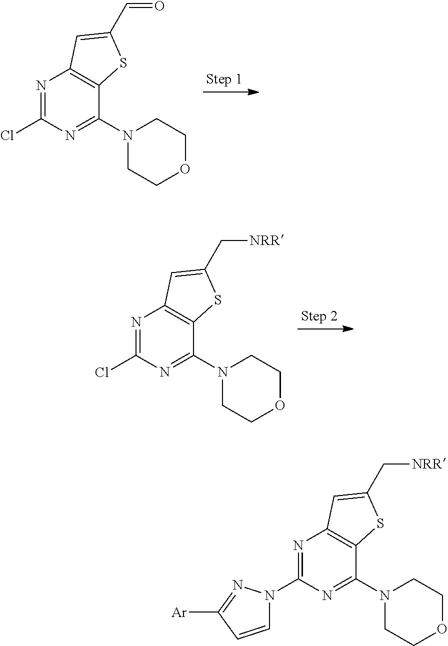

The compounds of Examples 9, 16, 30, 34, and 47 are prepared according to the following scheme:

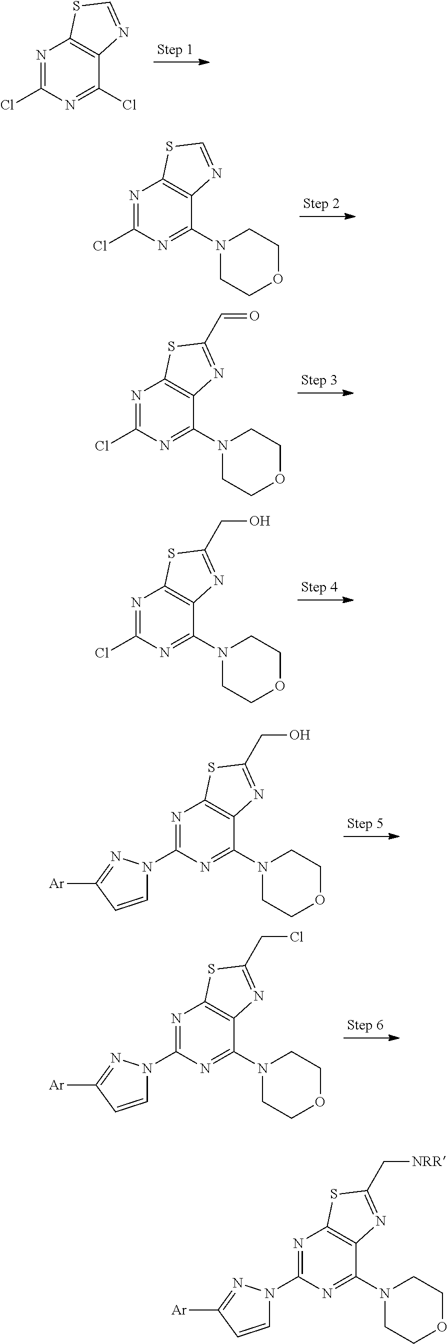

The compounds of Examples 10, 11, 13, and 14, are prepared according to the following scheme:

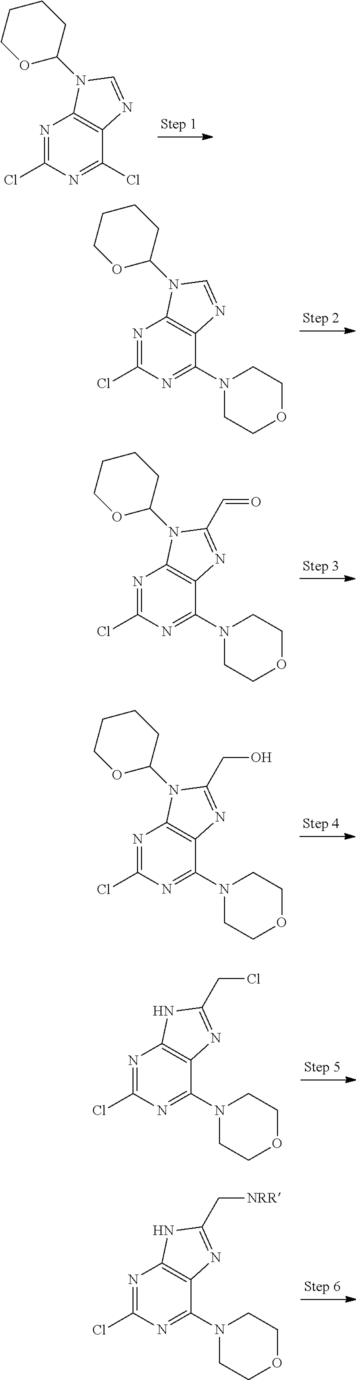

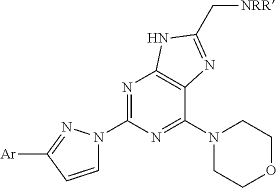

The compounds of Examples 15, 35, 36, 40, and 43, are prepared according to the following scheme:

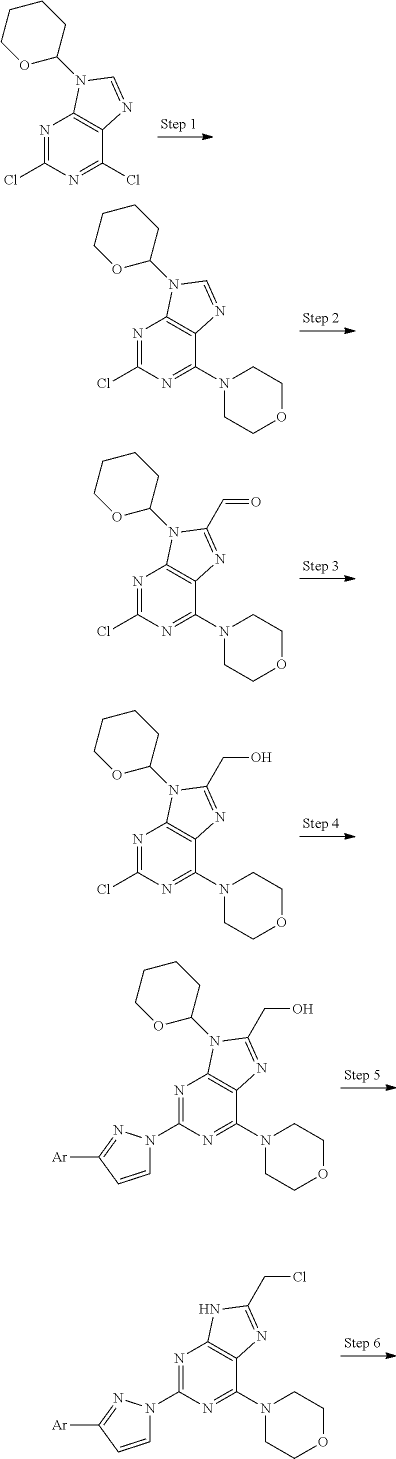

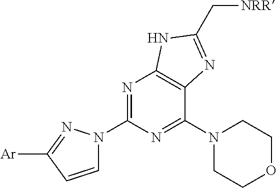

The compound of Example 12 is prepared according to the following scheme:

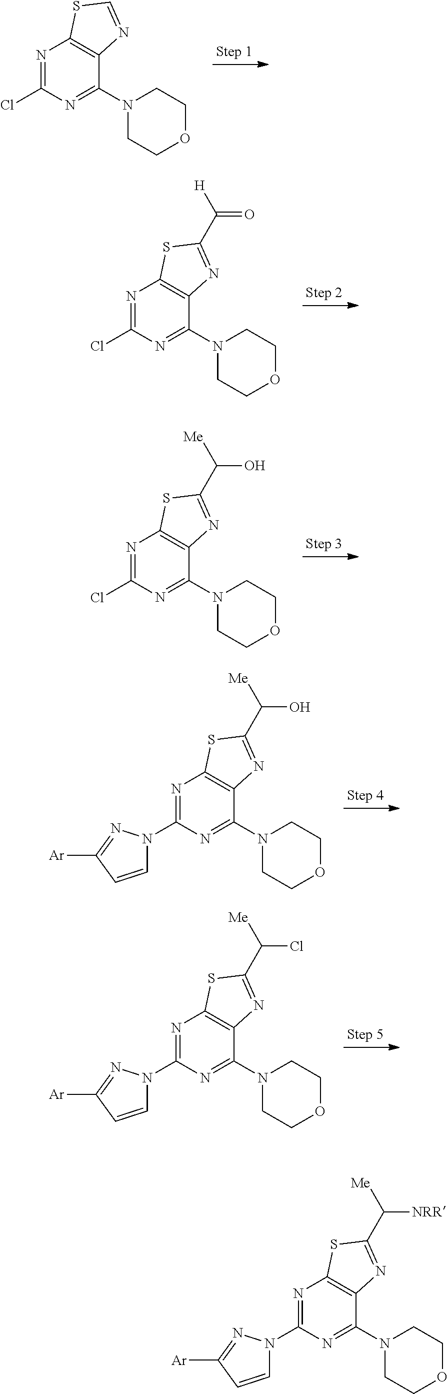

The compounds of Examples 17 and 26, are prepared according to the following scheme:

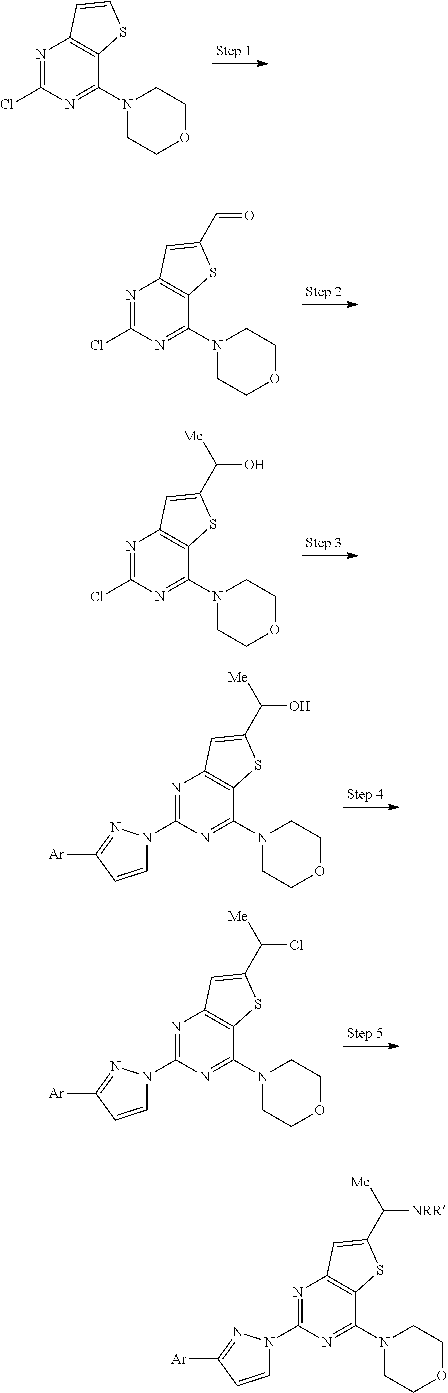

The compounds of Examples 18, 19, 20, 29, 31, 32, 33, 45, 50, and 51 are prepared according to the following scheme:

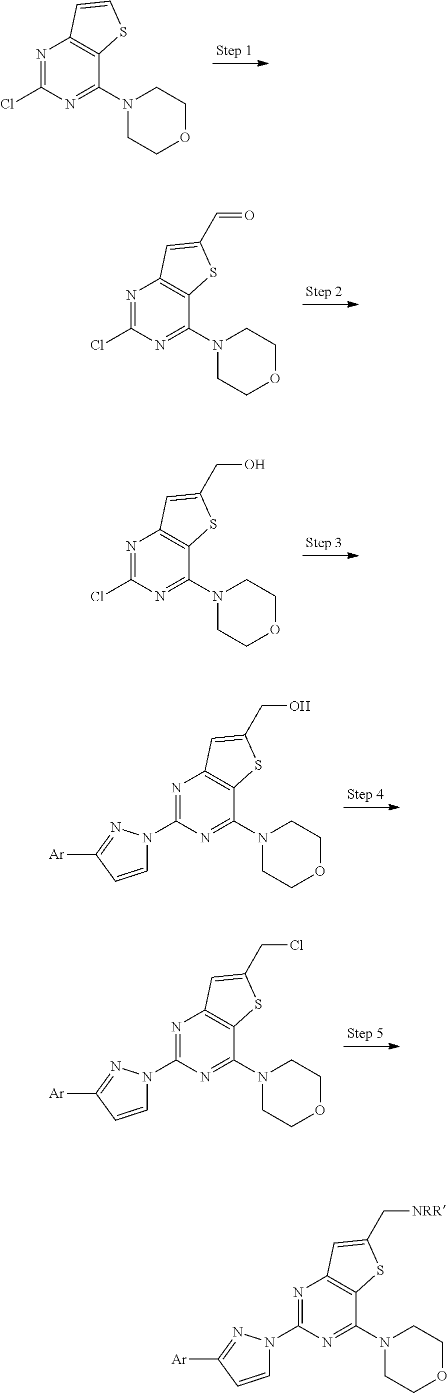

The compounds of Examples 21, 22, 23, 24, and 25, are prepared according to the following scheme:

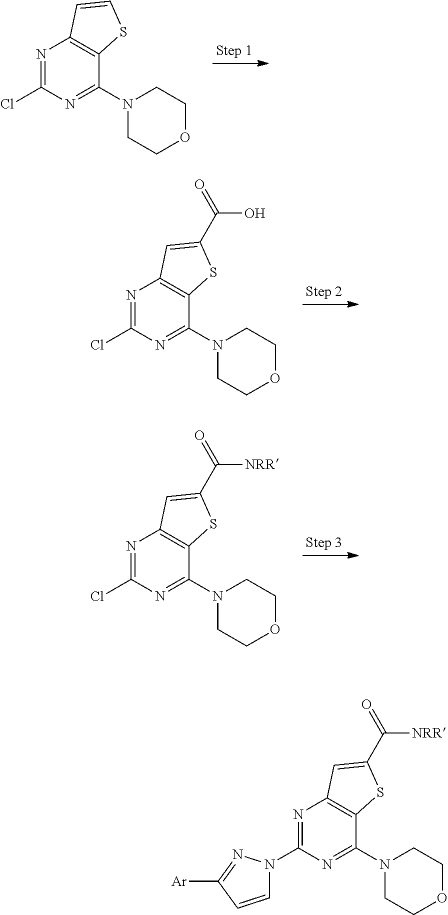

The compounds of Examples 27, 28, 38, 39, 41, 42, 46, 48, and 52, are prepared according to the following schemes:

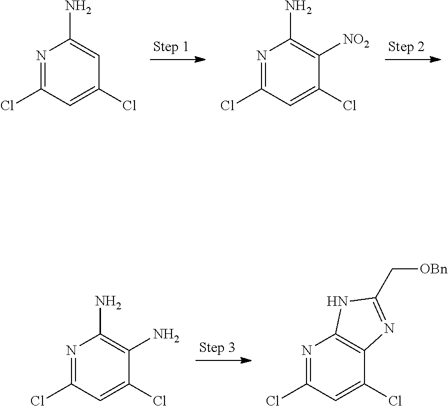 [SMILES: Nc1cc(Cl)cc(Cl)n1 ; Nc1nc(Cl)cc(Cl)c1[N+](=O)[O-] ; Nc1nc(Cl)cc(Cl)c1N ; Clc1cc(Cl)c2nc(COCc3ccccc3)[nH]c2n1]

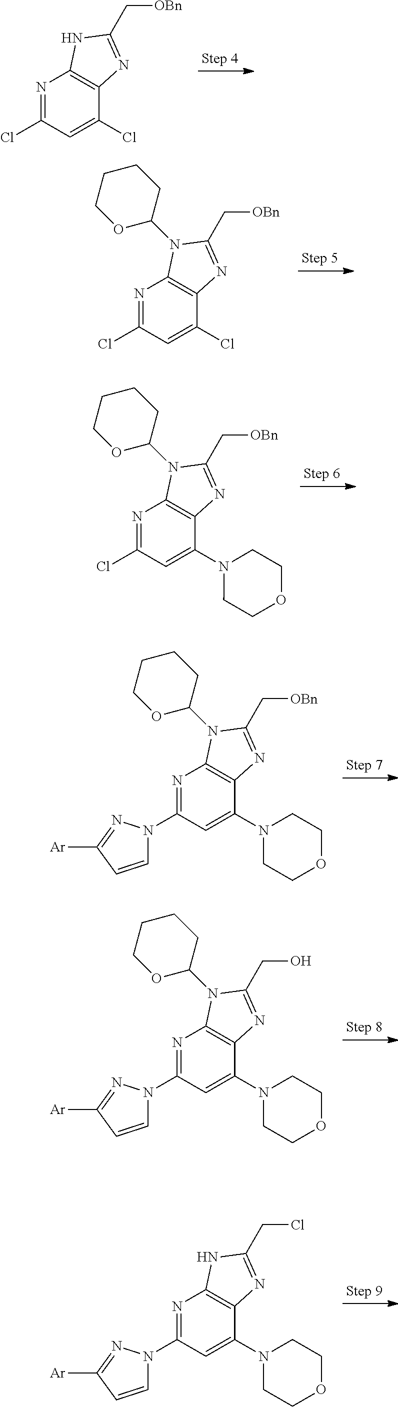

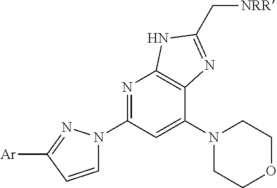

The compound of Example 37 is prepared according to the following scheme

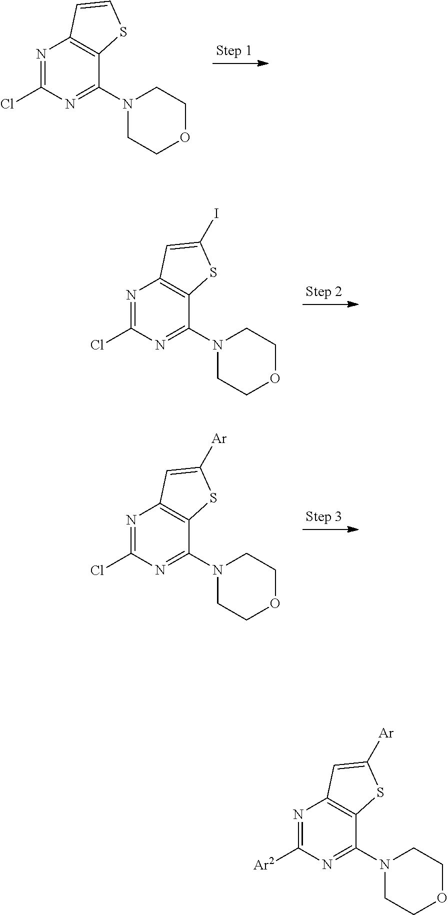

The compound of Example 44 is prepared according to the following scheme:

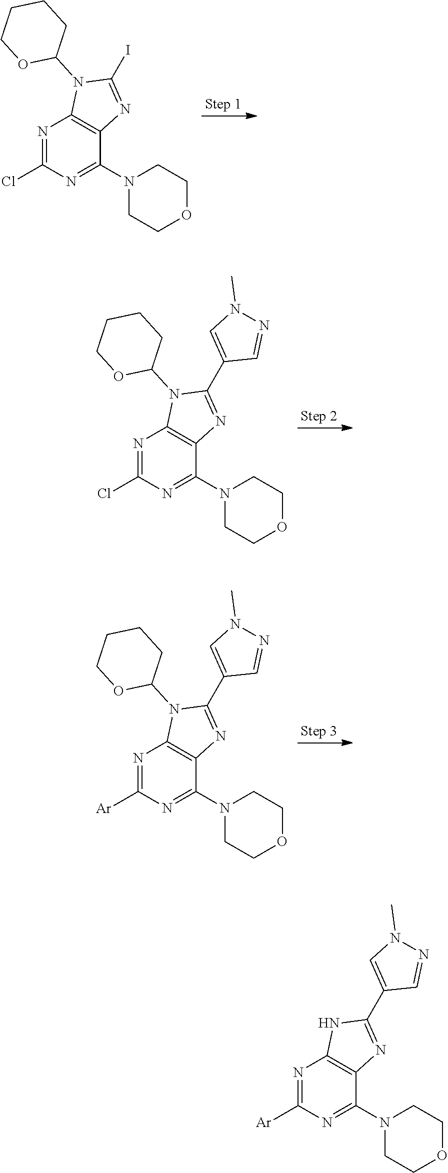

The compound of Example 49 is prepared according to the following scheme:

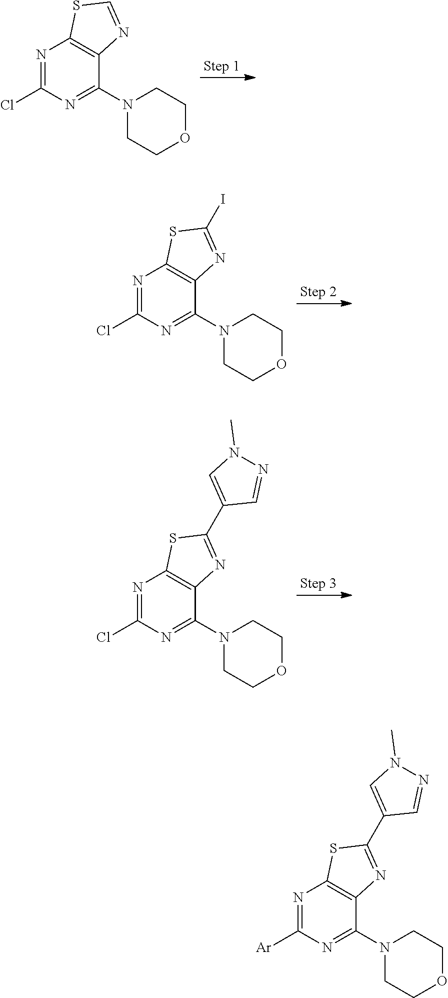

The compounds of Examples 53, 54, 55, 58, and 63, are prepared according to the following scheme:

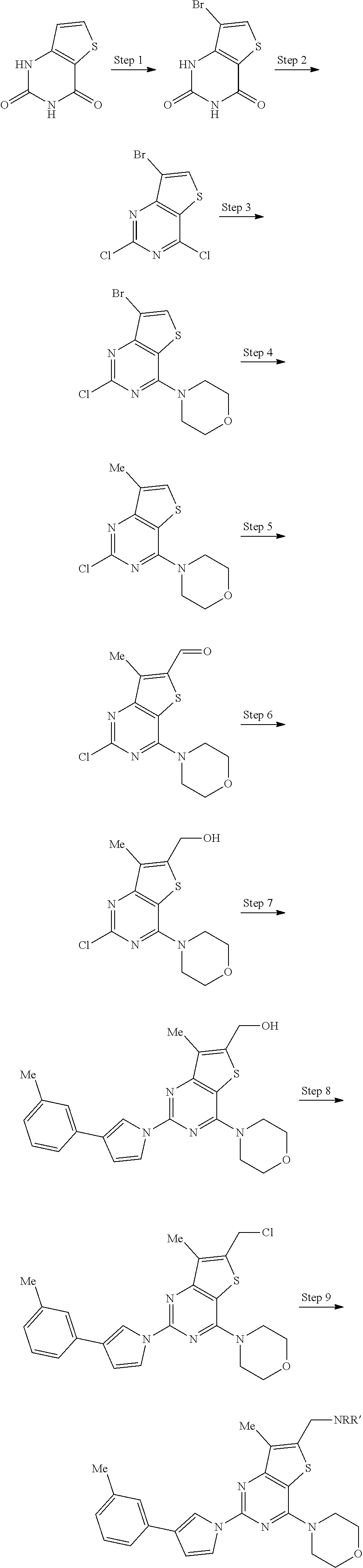

The compounds of Examples 56, 57, 62, and 171 are prepared according to the following scheme:

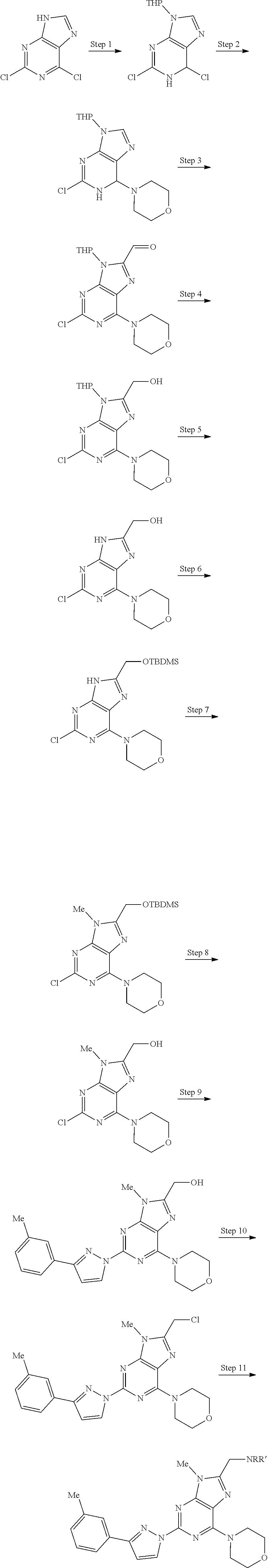

The compounds of Examples 59, 60, and 61, are prepared according to the following scheme:

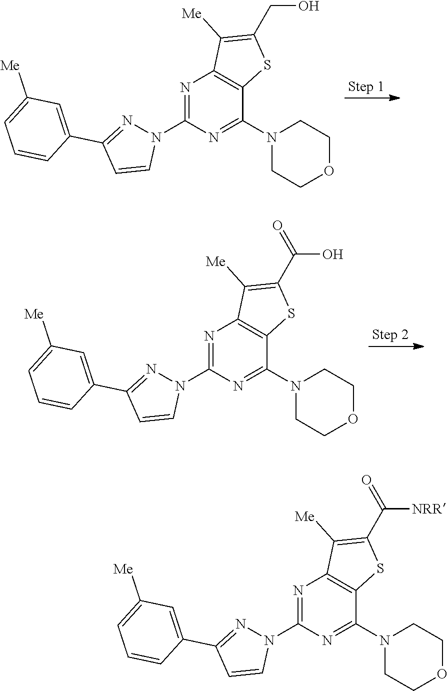

The compound of Example 64 is prepared according to the following scheme:

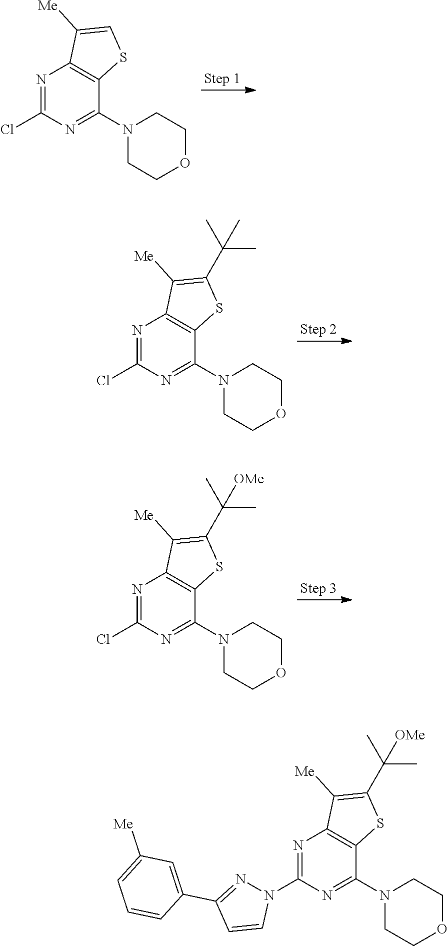 [SMILES: Cc1csc2c(N3CCOCC3)nc(Cl)nc12 ; Cc1c(C(C)(C)C)sc2c(N3CCOCC3)nc(Cl)nc12 ; COC(C)(C)c1sc2c(N3CCOCC3)nc(Cl)nc2c1C ; COC(C)(C)c1sc2c(N3CCOCC3)nc(-n3ccc(-c4cccc(C)c4)n3)nc2c1C]

The compound of Example 65 is prepared according to the following scheme:

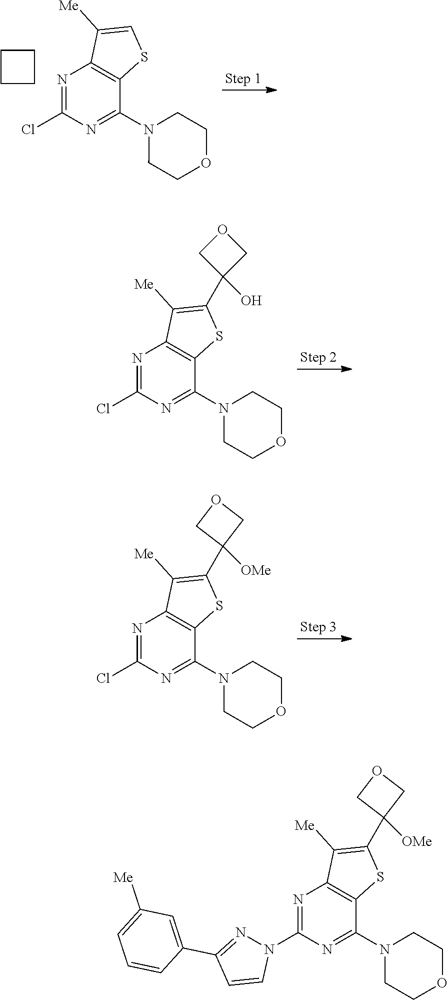

The compounds of Examples 66, 67, 68, 69, and 70, are prepared according to the following scheme:

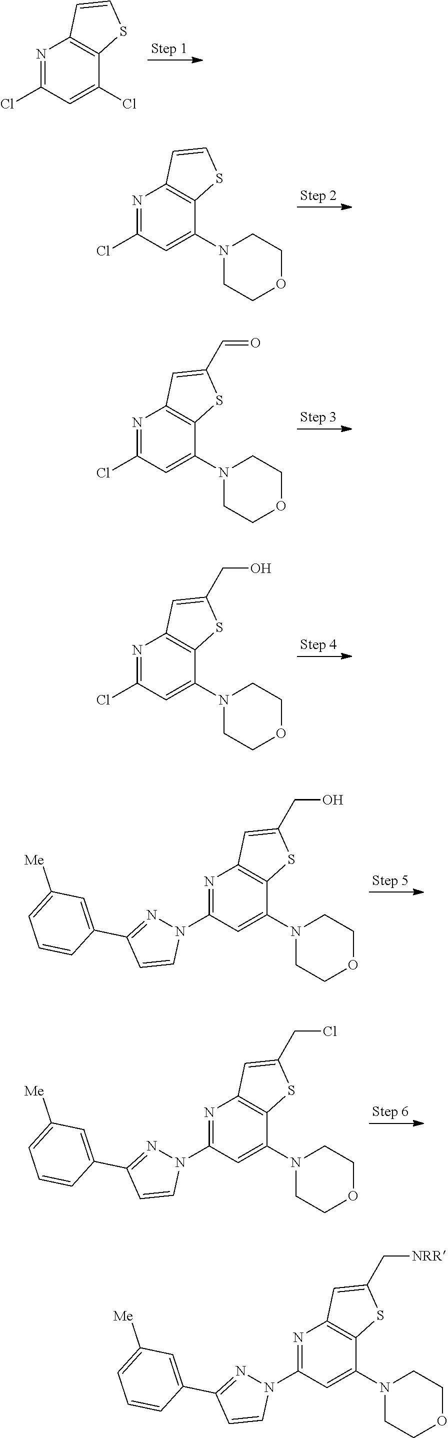

The compound of Example 71 is prepared according to the following scheme:

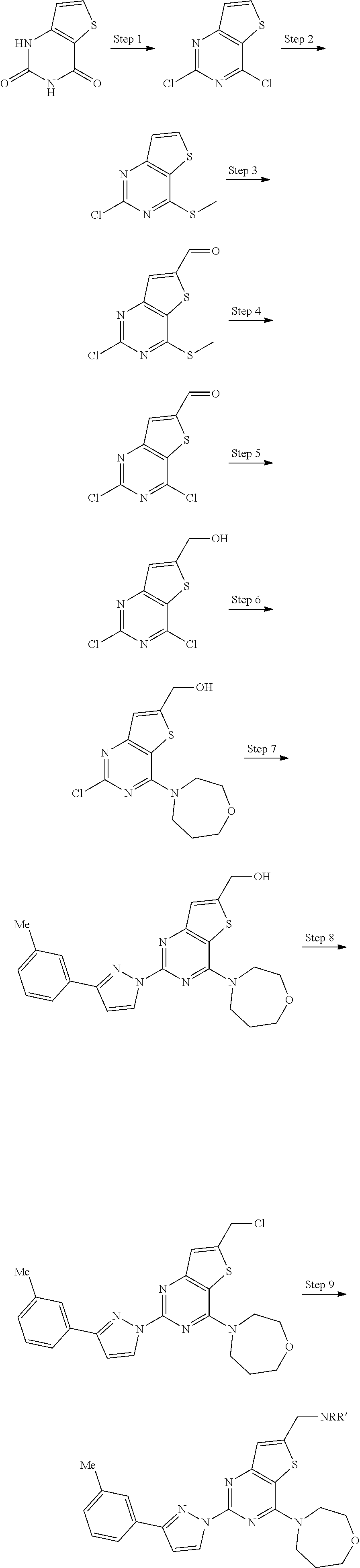

The compounds of Examples 72, 74, 75, 76, 79, 82, and 83 are prepared according to the following scheme:

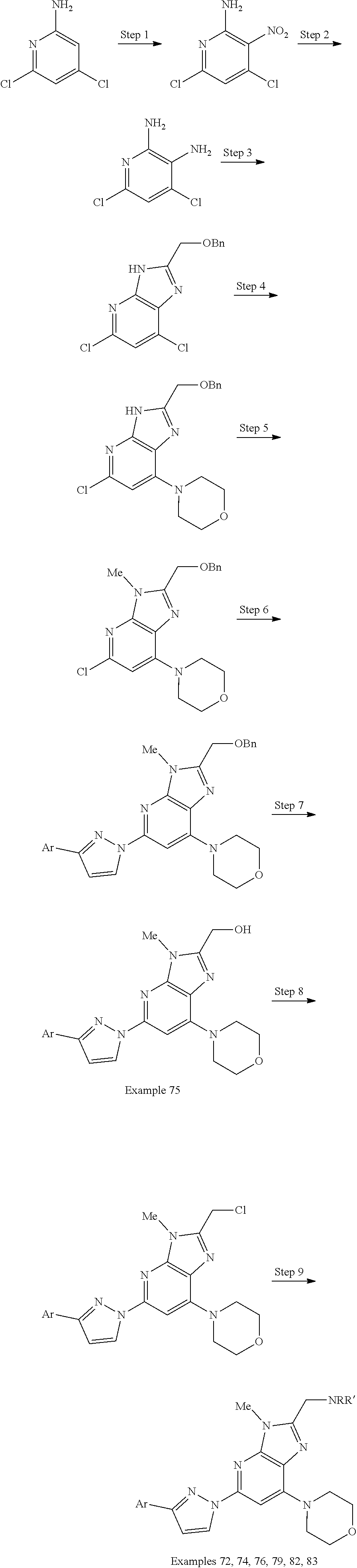

The compound of Example 73 is prepared according to the following scheme, which begins from the product of Step 5 of the scheme for Example 71:

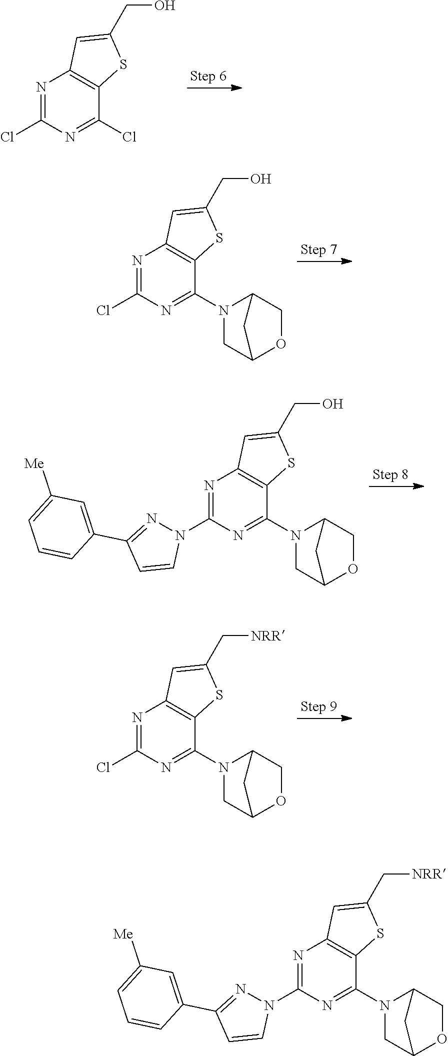

The compound of Example 77 is prepared according to the following scheme, which begins from the product of Step 5 of the scheme for Example 71:

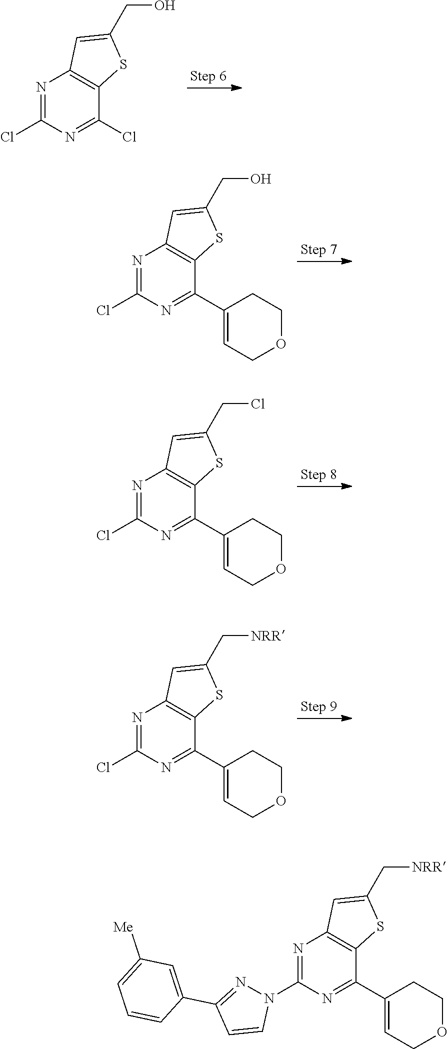

The compounds of Examples 78, 84, 85, and 86 are prepared according to the following scheme:

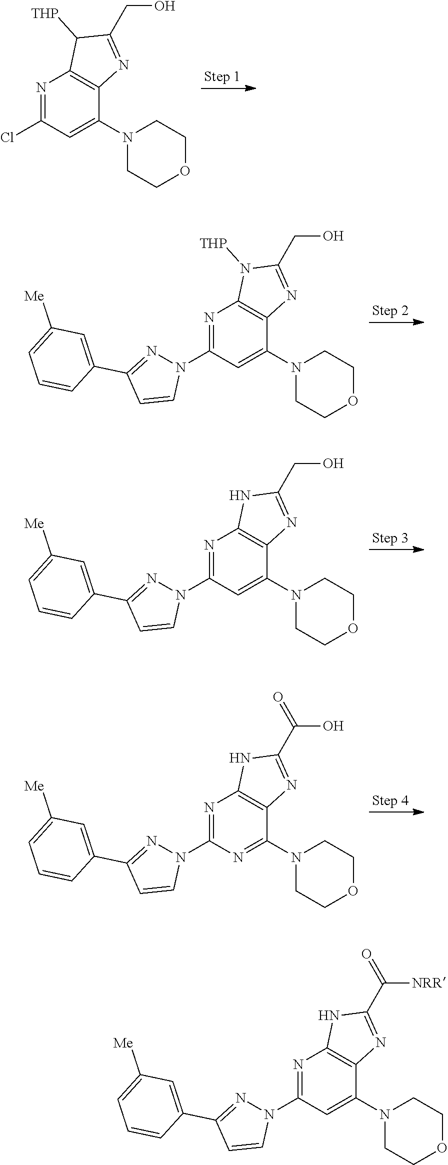

The compound of Example 129 is prepared according to the following scheme, which begins from the product of Step 5 of the scheme for Example 71:

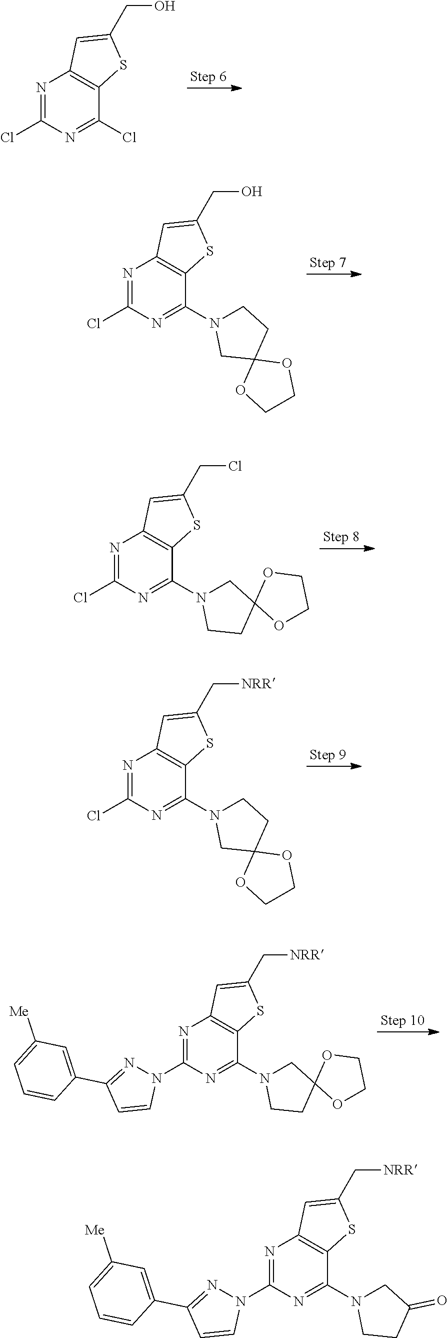

The compound of Example 165 is prepared according to the following scheme:

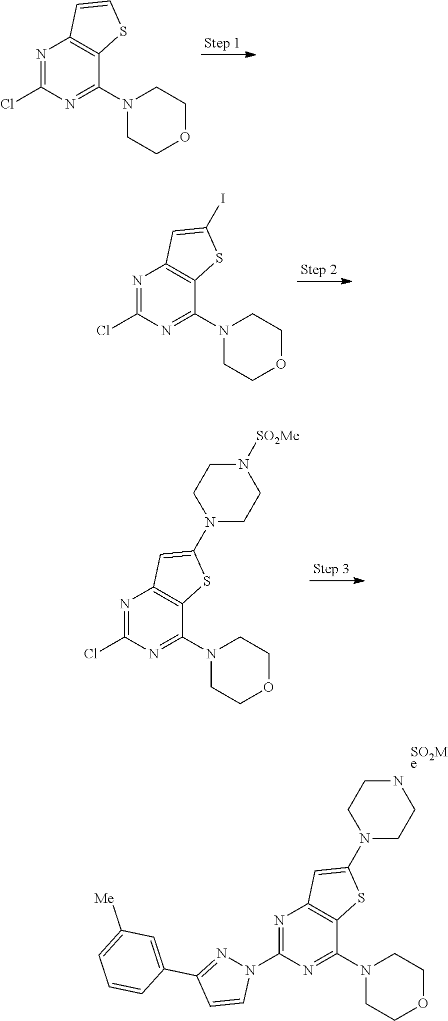 [SMILES: Clc1nc(N2CCOCC2)c2sccc2n1 ; Clc1nc(N2CCOCC2)c2sc(I)cc2n1 ; CS(=O)(=O)N1CCN(c2cc3nc(Cl)nc(N4CCOCC4)c3s2)CC1 ; Cc1cccc(-c2ccn(-c3nc(N4CCOCC4)c4sc(N5CCNCC5)cc4n3)n2)c1 ; "SO2Me"]

The compound of Example 166 is prepared according to the following scheme:

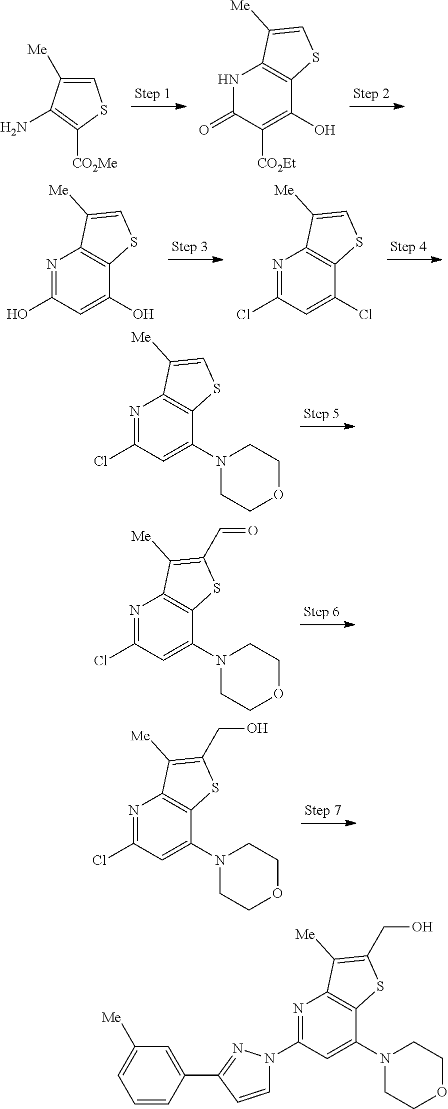

From the compound of Example 166, the compounds of Examples 132, 167, 168, 169, 170, and 172 are prepared according to the following scheme:

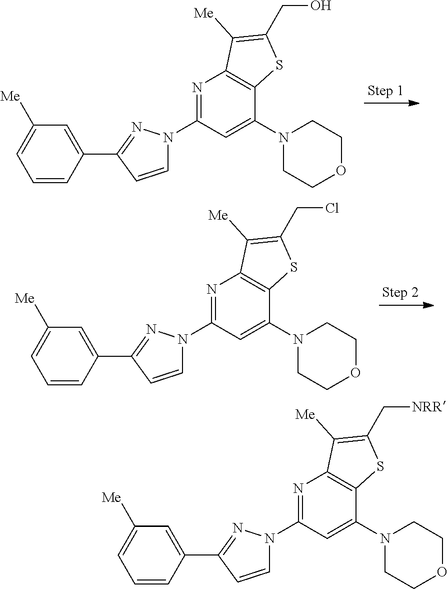

The compound of Example 173 is prepared according to the following scheme:

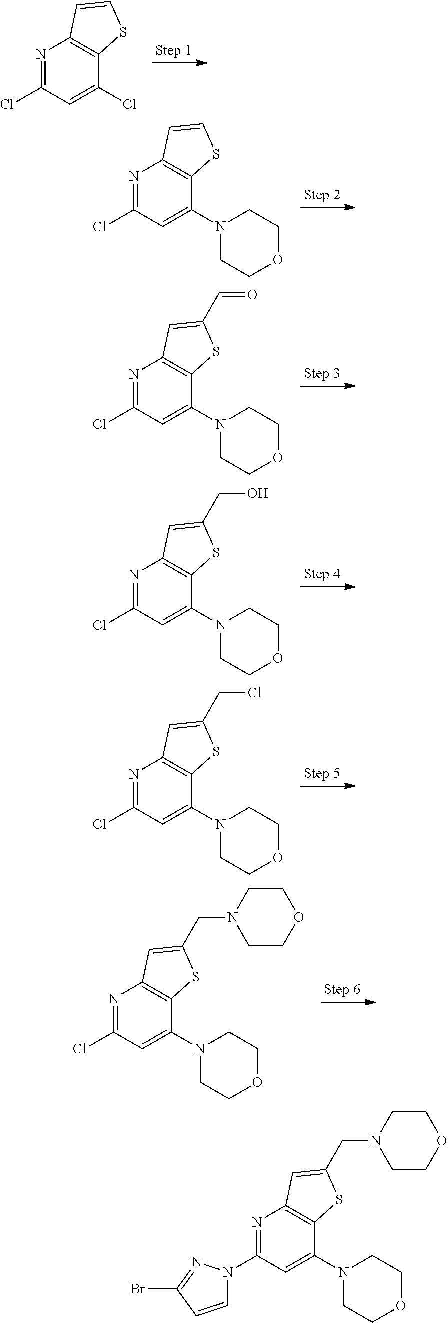

From the compound of Example 173, the compounds of Examples 178, 184, 189, 190, 191, 192 and 193, are prepared according to the following scheme:

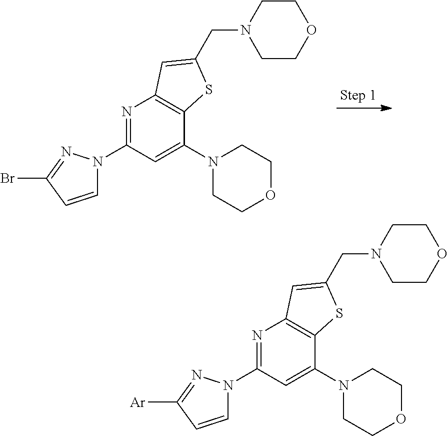

The compounds of Examples 174, 175, and 176, are prepared according to the following scheme:

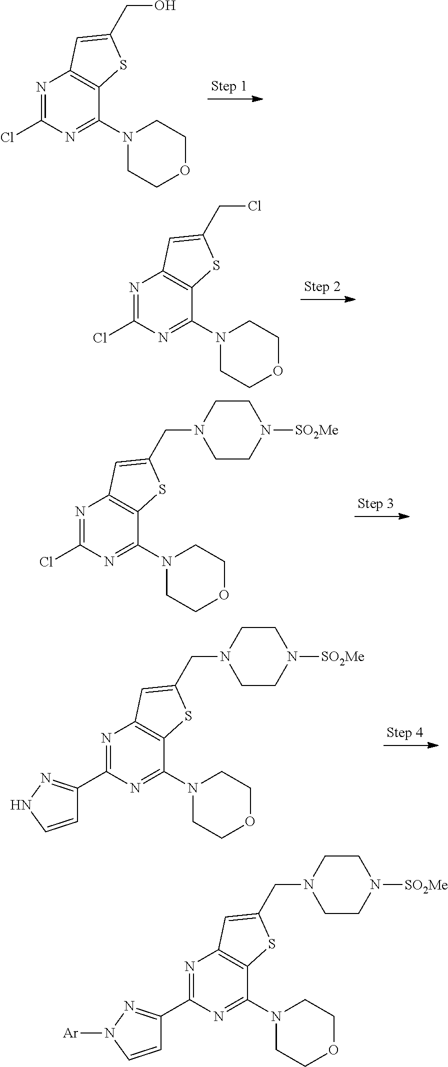

The compounds of Examples 177, 179, 183, 185, 186, 187, 198, 199, 200, and 201 are prepared according to the following scheme:

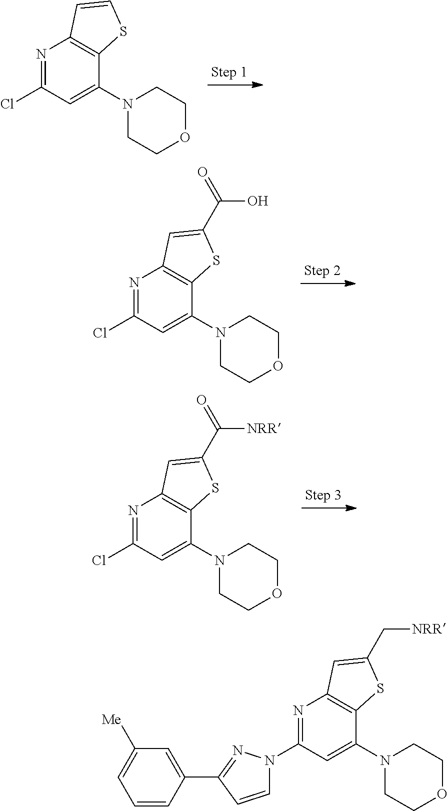

The compounds of Examples 181, 188, 194, 204, 205, 206, and 207, are prepared according to the following scheme:

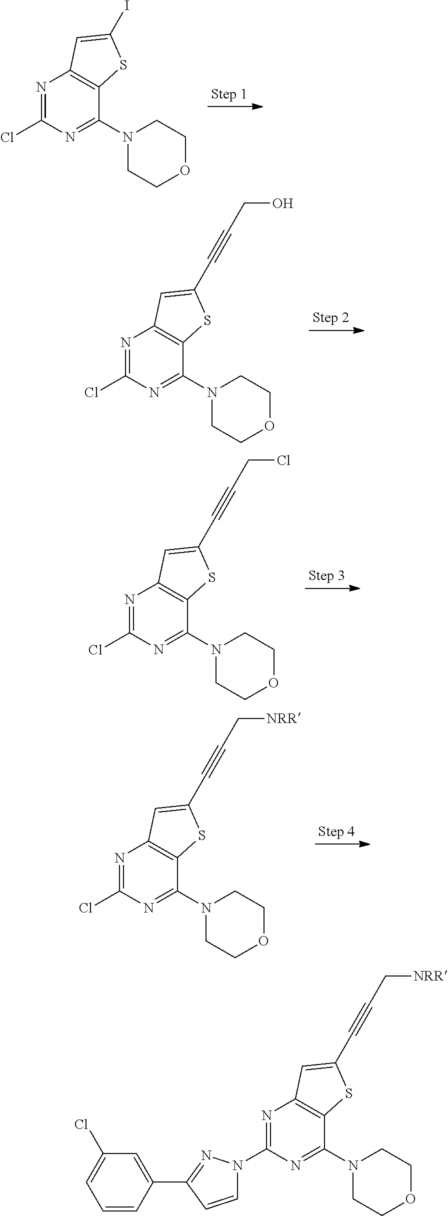

The compounds of Examples 182, 195, 196, 197, 202, 203, 208, and 209, are prepared according to the following scheme:

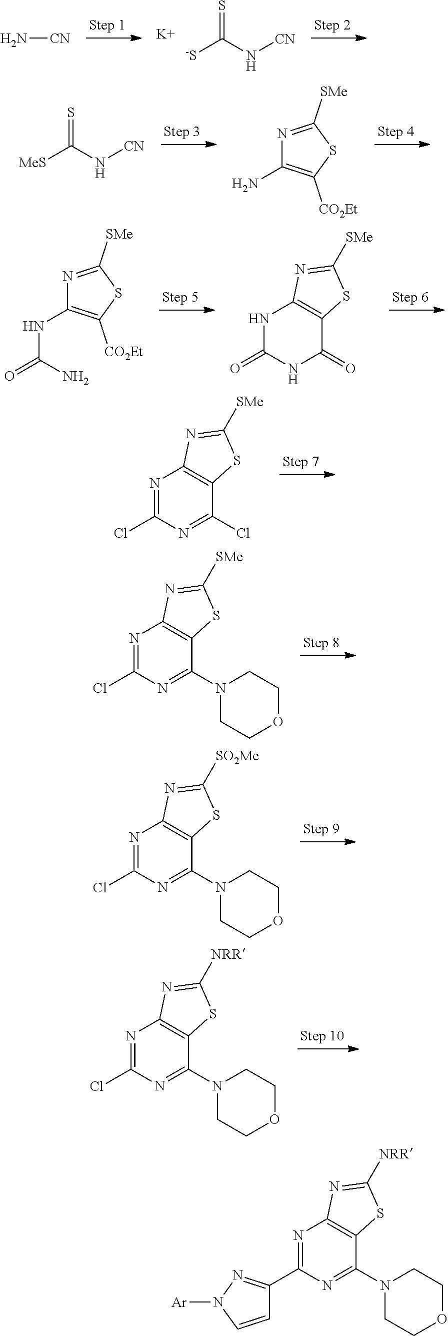

The compounds of Examples 210, 212, 213, 214, 215, and 216, are prepared according to the following scheme:

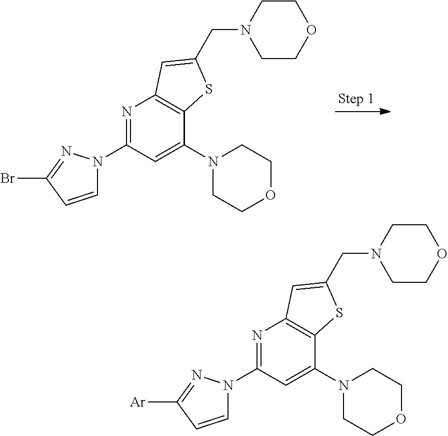

The compound of Example 211 is prepared according to the following scheme:

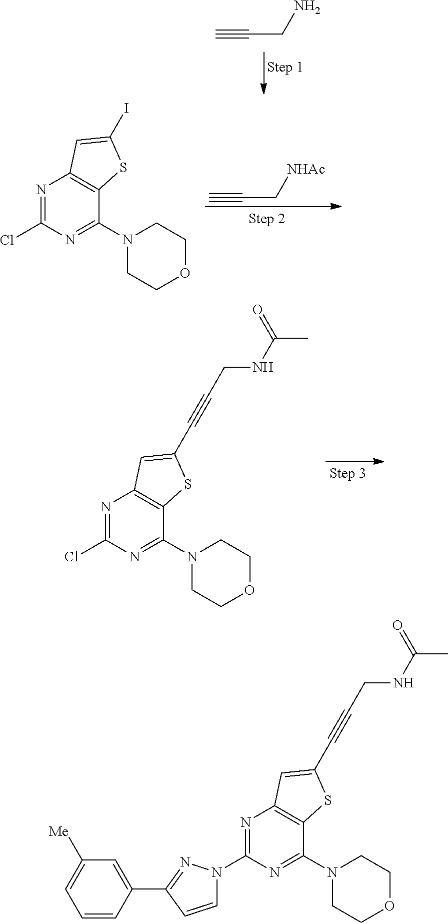

The compound of Example 217 is prepared according to the following scheme:

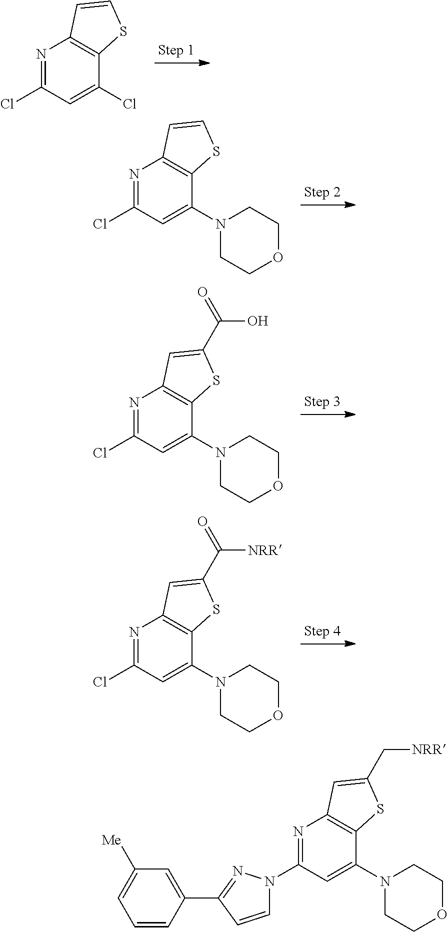

The compound of Example 218 is prepared according to the following scheme:

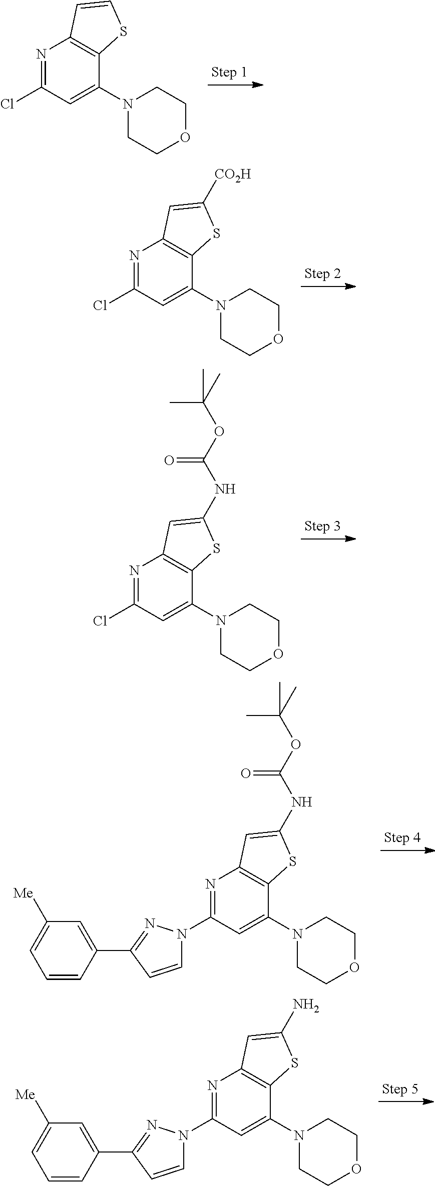

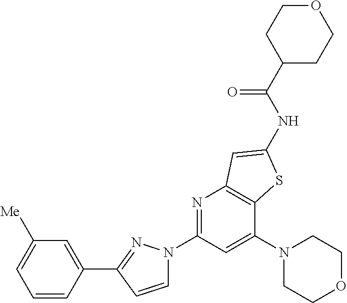 [SMILES: Cc1cccc(-c2ccn(-c3cc(N4CCOCC4)c4sc(NC(=O)C5CCOCC5)cc4n3)n2)c1]

The compounds of Example 263 and 264 may be prepared according to the following scheme:

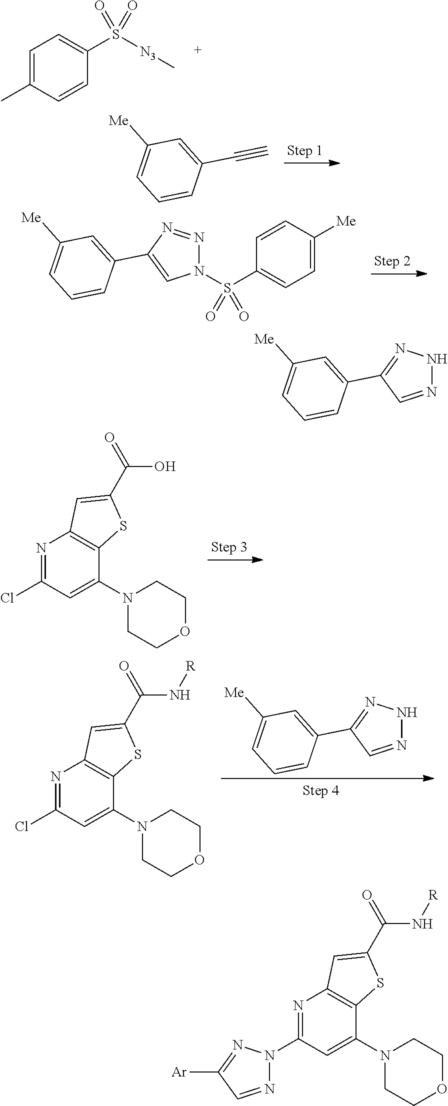

The compounds of Example 265 and 266 may be prepared according to the following scheme:

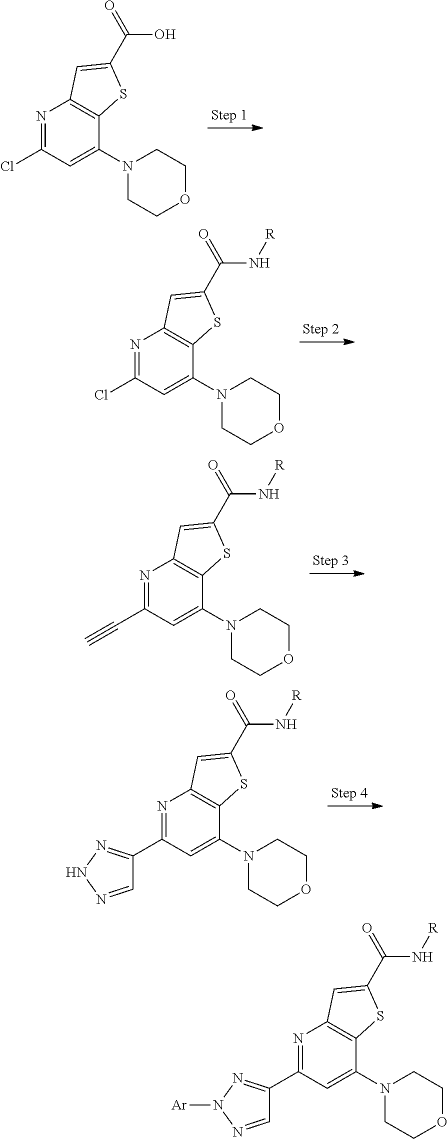

The compounds of Example 268 and 269 may be prepared according to the following scheme:

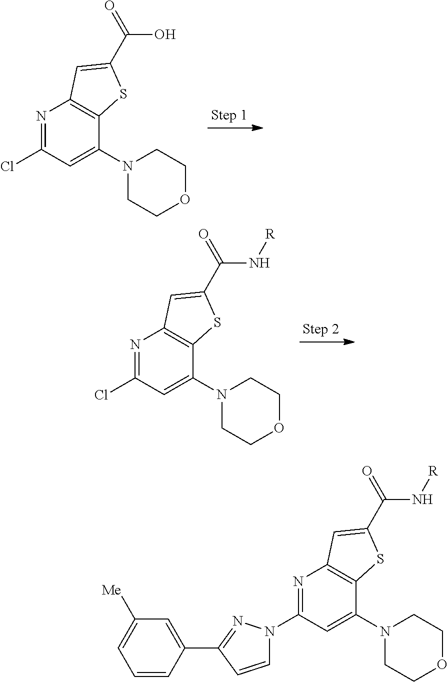

The compounds of Examples 87-131, 133-142, 144-164, 219-262, and 267 may be prepared according to analogous procedures to those set forth above and herein below.

### Example 1

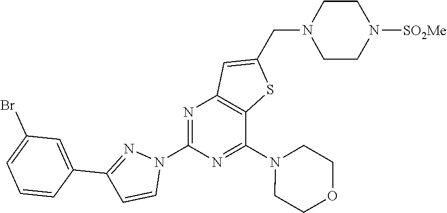 [SMILES: CS(=O)(=O)N1CCN(Cc2cc3nc(-n4ccc(-c5cccc(Br)c5)n4)nc(N4CCOCC4)c3s2)CC1]

### Step 1: 4-(2-chloro-6-((4-(methylsulfonyl)piperazin-1-yl)methyl)thieno[3,2-d]pyrimidin-4-yl)morpholine

To a stirred solution of 2-chloro-4-morpholinothieno[3,2-d]pyrimidine-6-carbaldehyde (200 mg, 0.704 mmol, 1 eq.) in chloroform (10 ml) cooled to 0° C. is added 1-(methylsulfonyl)piperazine (230 mg, 1.409 mmol, 2 eq.) followed by titanium tetraisopropoxide (0.6 ml, 1.056 mmol, 1.5 eq) and the reaction is allowed to stir at 65° C. for 12 hours (imine formation is monitored by TLC). The reaction mixture is evaporated under reduced pressure, and the obtained residue is re-dissolved in dichloroethane (DCE) (15 ml) and sodium cyanoborohydride is added (70 mg, 1.056 mmol, 1.5 eq.) at 0° C. The reaction mixture is stirred at room temperature for 4 hours. After completion of the reaction, the reaction mixture is filtered through a Celite pad, washed with water (10 mL) and extracted with DCM (3×20 mL). The combined organic layer is washed with brine solution (40 mL), dried over anhydrous sodium sulfate and evaporated under vacuum to obtain a crude product, which is further purified by silica gel (60-120 mesh) column chromatography using 3-5% MeOH:DCM as an eluents to afford the title product as a white solid (210 mg, 69%). ¹H-NMR (400 MHz, DMSO-d₆): δ 7.19 (s, 1H), 3.99-3.97 (m, 4H), 3.85-3.82 (m, 6H), 3.29 (t, J=4.8 Hz, 4H), 2.80 (s, 3H), 2.66 (t, J=4.8 Hz, 4H). Mass (m/z): 432.0 [M+H]⁺.

Step 2: 4-(2-(3-(3-bromophenyl)-1H-pyrazol-1-yl)-6-((4-(methylsulfonyl)piperazin-1-yl)methyl)thieno[3,2-d]pyrimidin-4-yl)morpholine: To a stirred suspension of sodium hydride (57-63%) (7 mg, 0.174 mmol, 1.5 eq.) in THE (6 mL) is added 3-(3-bromophenyl)-1H-pyrazole (28 mg, 0.127 mmol, 1.1 eq.) at 0° C. under nitrogen atmosphere, and the mixture is stirred at the same temperature for 30 min. To this mixture, is added 4-(2-chloro-6-((4-(methylsulfonyl)piperazin-1-yl)methyl)thieno[3,2-d]pyrimidin-4-yl)morpholine (50 mg, 0.116 mmol, 1 eq.) and the reaction is allowed to stir at 120° C. under microwave conditions (MW) for 2 hours. After completion, the reaction mixture is quenched with ice-cold water (20 mL) and extracted with ethyl acetate (2×20 mL). The combined organic layer is washed brine solution (10 mL), dried over anhydrous sodium sulfate and evaporated under reduced pressure to obtain the crude residue, which is purified from by preparative HPLC to give the product as a white fluffy solid (6 mg, 5%). ¹H-NMR (400 MHz, DMSO-d₆): δ 8.76 (d, J=2.8 Hz, 1H), 8.14 (s, 1H), 7.98 (d, J=8 Hz, 1H), 7.58 (d, J=8.8 Hz, 1H), 7.47-7.42 (m, 2H), 7.14 (d, J=2.8 Hz, 1H), 4.01 (t, J=2.8 Hz, 4H), 3.95 (s, 2H), 3.80 (t, J=4.8 Hz, 4H), 3.19-3.13 (m, 4H), 2.90 (s, 3H), 2.63-2.59 (m, 4H). Mass (m/z): 619.9 [M+H]⁺.

### Example 2

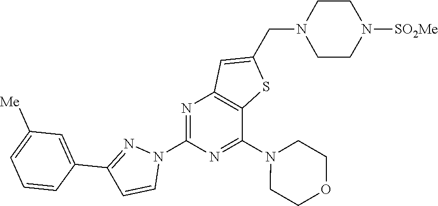 [SMILES: Cc1cccc(-c2ccn(-c3nc(N4CCOCC4)c4sc(CN5CCN(S(C)(=O)=O)CC5)cc4n3)n2)c1]

### Step 2: 4-(6-((4-(methylsulfonyl)piperazin-1-yl)methyl)-2-(3-(m-tolyl)-1H-pyrazol-1-yl)thieno[3,2-d]pyrimidin-4-yl)morpholine

To a stirred suspension of sodium hydride (57-63%) (5.5 mg, 0.139 mmol, 1.5 eq.) in THE (5 mL) is added 3-(m-tolyl)-1H-pyrazole (16 mg, 0.102 mmol, 1.1 eq.) at 0° C. under nitrogen atmosphere and stirred at the same temperature for 30 minutes. Then 4-(2-chloro-6-((4-(methylsulfonyl)piperazin-1-yl)methyl)thieno[3,2-d]pyrimidin-4-yl)morpholine (40 mg, 0.09 mmol, 1 eq.) is added, and the reaction mixture is allowed to stir at 120° C. under microwave (MW) conditions for 2 hours. After completion, the reaction mixture is quenched with ice-cold water (20 mL) and extracted with ethyl acetate (2×20 mL). The combined organic layer is washed brine solution (10 mL), dried over anhydrous sodium sulfate and evaporated under reduced pressure to give the crude product, which is purified by preparative HPLC to give the product as a white fluffy solid (11 mg, 22%). ¹H-NMR (400 MHz, DMSO-d₆): δ 8.73 (d, J=2.8 Hz, 1H), 7.79 (s, 1H), 7.75 (d, J=7.6 Hz, 1H), 7.44 (s, 1H), 7.36 (t, J=7.6 Hz, 1H), 7.20 (d, J=7.6 Hz, 1H), 7.03 (d, J=2.8 Hz, 1H), 4.01 (t, J=4.4 Hz, 4H), 3.95 (s, 2H), 3.80 (t, J=4.8 Hz, 4H), 3.18-3.11 (m, 4H), 2.90 (s, 3H), 2.54-2.47 (m, 4H), 2.39 (s, 3H). Mass (m/z): 554.0 [M+H]⁺.

### Example 3

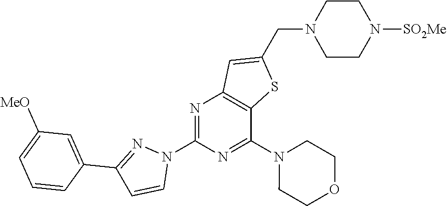 [SMILES: COc1cccc(-c2ccn(-c3nc(N4CCOCC4)c4sc(CN5CCN(S(C)(=O)=O)CC5)cc4n3)n2)c1]

### Step 2: 4-(2-(3-(3-methoxyphenyl)-1H-pyrazol-1-yl)-6-((4-(methylsulfonyl)piperazin-1-yl)methyl)thieno[3,2-d]pyrimidin-4-yl)morpholine

To a stirred suspension of sodium hydride (57-63%) (7 mg, 0.174 mmol, 1.5 eq.) in THE (7 mL) is added 3-(3-methoxyphenyl)-1H-pyrazole (23 mg, 0.127 mmol, 1.1 eq.) at 0° C. under nitrogen atmosphere and stirred at the same temperature for 30 min. Then 4-(2-chloro-6-((4-(methylsulfonyl)piperazin-1-yl)methyl)thieno[3,2-d]pyrimidin-4-yl)morpholine (50 mg, 0.116 mmol, 1 eq.) is added, and the reaction is allowed to stir at 120° C. under microwave (MW) conditions for 2 hours. After completion, the reaction mixture is quenched with ice-cold water (20 mL) and extracted with ethyl acetate (3×30 mL). The combined organic layer is washed with brine solution (30 mL), dried over anhydrous sodium sulfate and evaporated under reduced pressure to obtain the crude residue, which is purified by preparative HPLC to give the product as a white solid (12 mg, 18%). ¹H-NMR (400 MHz, DMSO-d₆): δ 8.73 (d, J=2.4 Hz, 1H), 7.54 (d, J=8.0 Hz, 1H), 7.49-7.48 (m, 1H), 7.44 (s, 1H), 7.39 (t, J=8 Hz, 1H), 7.06 (d, J=2.8 Hz, 1H), 6.96 (dd, J=8.0, 2.0 Hz, 1H), 4.01 (t, J=4.4 Hz, 4H), 3.95 (s, 2H), 3.84 (s, 3H), 3.81 (t, J=4.4 Hz, 4H), 3.16 (t, J=4.8 Hz, 4H), 2.90 (s, 3H), 2.61 (t, J=4.4 Hz, 4H). Mass (m/z): 570.0 [M+H]⁺.

### Example 4

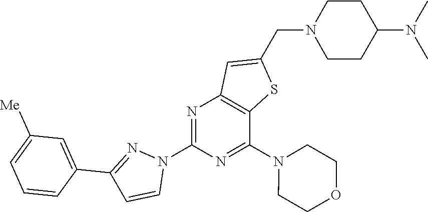 [SMILES: Cc1cccc(-c2ccn(-c3nc(N4CCOCC4)c4sc(CN5CCC(N(C)C)CC5)cc4n3)n2)c1]

### Step 1: 1-((2-chloro-4-morpholinothieno[3,2-d]pyrimidin-6-yl)methyl)-N,N-dimethyl piperidin-4-amine

To a stirred solution of 2-chloro-4-morpholinothieno[3,2-d]pyrimidine-6-carbaldehyde (50 mg, 0.176 mmol, 1 eq) in chloroform (5 ml) at 0° C. is added N,N-dimethylpiperidin-4-amine (46 mg, 0.353 mmol, 2 eq) followed by titanium isopropoxide (60 μL, 0.264 mmol, 1.5 eq). The reaction mixture is stirred at 65° C. for 12 hours. After completion of the reaction, it is evaporated under reduced pressure to obtain a crude compound. This is dissolved in dichloroethane (DCE) (10 ml), and then sodium cyanoborohydride (13 mg, 0.264 mmol, 1.5 eq) is added at 0° C. The reaction mixture is allowed to warm to room temperature and it is stirred for 12 hours. After completion of the reaction, it is filtered through a Celite pad, followed by washing of the celite pad with DCM (50 mL). The filtrate is washed with water (10 mL). The organic layer is washed with brine (10 mL), dried over anhydrous sodium sulfate, filtered and evaporated under reduced pressure to obtain the crude title compound as a brown syrup. Mass (m/z): 396.2 [M+H]⁺.

### Step 2: N, N-dimethyl-1-((4-morpholino-2-(3-(m-tolyl)-1H-pyrazol-1-yl)thieno[3,2-d]pyrimidin-6-yl)methyl)piperidin-4-amine

To a stirred suspension of sodium hydride (57-63%) (3.5 mg, 0.094 mmol, 1.5 eq) in THE (7 mL) is added 3-(m-tolyl)-1H-pyrazole (10 mg, 0.063 mmol, 1.1 eq) at 0° C. under nitrogen atmosphere and the mixture is stirred for 30 minutes. Then 1-((2-chloro-4-morpholinothieno[3,2-d]pyrimidin-6-yl)methyl)-N, N-dimethylpiperidin-4-amine (25 mg, 0.063 mmol, 1 eq) is added at the same temperature. The reaction mixture is stirred at 120° C. under microwave irradiation for 2 hours. After completion of the reaction, it is quenched with ice-cold water (5 mL) and extracted with ethyl acetate (3×10 mL). The combined organic layers are washed with brine solution (5 mL), dried over anhydrous sodium sulfate, filtered and evaporated under reduced pressure to provide a crude product, which is purified by preparative HPLC to give the title compound as a white fluffy solid. ¹H NMR (400 MHz, DMSO-d₆): 8.72 (d, J=4 Hz, 1H), 7.79 (bs, 1H), 7.75 (d, J=7.6 Hz, 1H), 7.39 (s, 1H), 7.37 (t, J=15.2 Hz, 1H), 7.20 (d, J=7.6 Hz, 1H), 7.02 (d, J=2.8 Hz, 1H), 4.01-3.99 (m, 4H), 3.83-3.79 (m, 6H), 2.95-2.92 (b s, 1H), 2.39 (s, 3H), 2.17 (s, 6H), 2.10 (t, J=10.8 Hz, 3H), 1.75-1.72 (d, J=11.2 Hz, 2H), 1.45-1.40 (m, 2H). Mass (m/z): 518.2 [M+H]⁺.

### Example 5

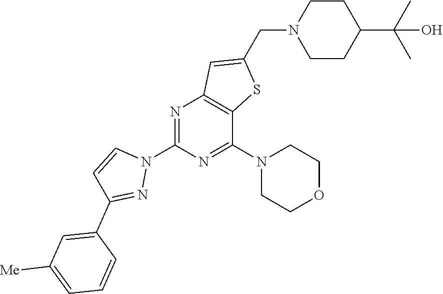 [SMILES: Cc1cccc(-c2ccn(-c3nc(N4CCOCC4)c4sc(CN5CCC(C(C)(C)O)CC5)cc4n3)n2)c1]

Step 1: 2-(1-((2-chloro-4-morpholinothieno[3,2-d]pyrimidin-6-yl)methyl)piperidin-4-yl)propan-2-ol

The title compound is made according to the procedure used for Example 1, with 2-(piperidin-4-yl)propan-2-ol in place of 1-(methylsulfonyl)piperazine.

### Step 2: 2-(1-((4-morpholino-2-(3-(m-tolyl)-1H-pyrazol-1-yl)thieno[3,2-d]pyrimidin-6-yl)methyl)piperidin-4-yl)propan-2-ol

According to the procedure used for Example 1, 2-(1-((2-chloro-4-morpholinothieno[3,2-d]pyrimidin-6-yl)methyl)piperidin-4-yl)propan-2-ol (25 mg) is reacted with 3-(m-tolyl)-1H-pyrazole (8 mg) to obtain the title compound (8 mg; 25%) as a white solid. Mass [m/z]=533.3 [M+H]⁺; ¹H-NMR (400 MHz, DMSO-d₆): δ 8.71 (d, J=2.4 Hz, 1H), 7.79 (s, 1H), 7.73 (d, J=8 Hz, 1H), 7.39 (s, 1H), 7.35 (t, J=7.6 Hz, 1H), 7.18 (d, J=7.6 Hz, 1H), 7.01 (d, J=2.4 Hz, 1H) 4.06 (s, 1H), 4.00 (t, J=4.8 Hz, 4H), 3.79-3.81 (m, 4H), 2.97 (d, J=10.8 Hz, 2H), 2.97 (s, 3H), 1.98 (t, J=11.6 Hz, 2H), 1.65 (d, J=11.6 Hz, 2H), 1.20-1.29 (m, 4H), 1.04 (s, 5H).

### Example 6

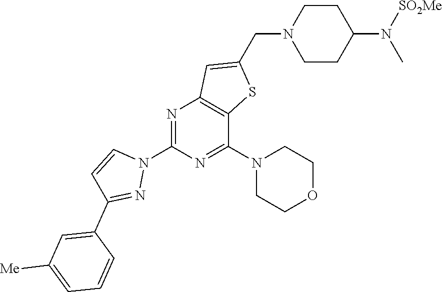 [SMILES: Cc1cccc(-c2ccn(-c3nc(N4CCOCC4)c4sc(CN5CCC(N(C)S(C)(=O)=O)CC5)cc4n3)n2)c1]

### Step 1: N-(1-((2-chloro-4-morpholinothieno[3,2-d]pyrimidin-6-yl)methyl)piperidin-4-yl)-N-methylmethanesulfonamide

The title compound is made according to the procedure used for Example 1, with N-(4-piperidinyl)-N-methylmethanesulfonamide in place of 1-(methylsulfonyl)piperazine.

### Step 2: N-methyl-N-(1-((4-morpholino-2-(3-(m-tolyl)-1H-pyrazol-1-yl)thieno[3,2-d]pyrimidin-6-yl)methyl)piperidin-4-yl)methanesulfonamide

According to the procedure used for Example 1, N-(1-((2-chloro-4-morpholinothieno[3,2-d]pyrimidin-6-yl)methyl)piperidin-4-yl)-N-methylmethanesulfonamide (30 mg) is reacted with 10 mg of 3-(m-tolyl)-1H-pyrazole (10 mg)e to obtain the title compound (9 mg; 24%) as a white solid. Mass [m/z]=533.3 [M+H]⁺; ¹H-NMR (400 MHz, DMSO-d₆): δ 8.72 (d, J 2.8 Hz, 1H), 7.79 (s, 1H), 7.73 (d, J 7.6 Hz, 1H), 7.41 (s, 1H), 7.35 (t, J 7.6 Hz, 1H), 7.18 (d, J=8 Hz, 1H), 7.02 (d, J=2.8 Hz, 1H) 4.00 (t, J=4.8 Hz, 4H), 3.87 (s, 2H), 3.79-3.80 (t, J=3.6 Hz, 4H), 3.56-3.62 (m, 1H), 2.97 (d, J=12.8 Hz, 2H), 2.90 (s, 3H), 2.71 (s, 3H), 2.39 (s, 3H), 2.17 (t, J=12.8, 2H), 1.73-1.77 (dd, J=11.6 Hz, 8.8 Hz, 2H), 1.60 (d, J=7.2 Hz, 2H).

### Example 7

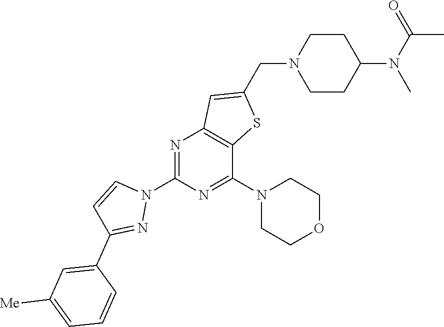 [SMILES: CC(=O)N(C)C1CCN(Cc2cc3nc(-n4ccc(-c5cccc(C)c5)n4)nc(N4CCOCC4)c3s2)CC1]

### Step 1: N-(1-((2-chloro-4-morpholinothieno[3,2-d]pyrimidin-6-yl)methyl)piperidin-4-yl)-N-methylacetamide

The title compound is made according to the procedure used for Example 1, with N-(4-piperidinyl)-N-methylacetamide in place of 1-(methylsulfonyl)piperazine.

### Step 2: N-methyl-N-(1-((4-morpholino-2-(3-(m-tolyl)-1H-pyrazol-1-yl)thieno[3,2-d]pyrimidin-6-yl)methyl)piperidin-4-yl)acetamide

According to the procedure used for Example 1, 25 mg of N-(1-((2-chloro-4-morpholinothieno[3,2-d]pyrimidin-6-yl)methyl)piperidin-4-yl)-N-methylacetamide (25 mg) is reacted with 14 mg of 3-(m-tolyl)-1H-pyrazole (14 mg) to obtain the title compound (4 mg; 12%) as a white solid. Mass [m/z]=546.2 [M+H]⁺; ¹H-NMR (400 MHz, DMSO-d₆): δ 8.75 (d, J 2.8 Hz, 1H), 7.79 (s, 1H), 7.74 (d, J 3.6 Hz, 1H), 7.35 (t, J 7.6 Hz, 1H), 7.21 (d, 1H), 7.05 (d, J=2.8 Hz, 1H), 4.68-4.72 (br s, 2H), 4.04 (t, 4H), 3.82 (t, J=4.8 Hz, 4H), 2.68 (s, 3H), 2.39 (s, 3H), 2.04 (s, 2H), 1.99 (s, 3H), 1.72 (br s, 1H).

### Example 9

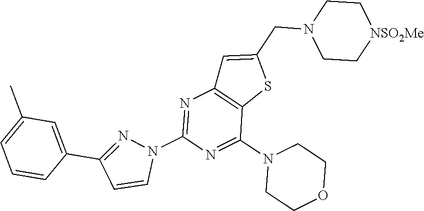

### Step 1: 4-(5-chlorothiazolo[5,4-d]pyrimidin-7-yl)morpholine

To a stirred solution of 5,7-dichlorothiazolo[5,4-d]pyrimidine (200 mg, 0.97 mmol, 1 eq) in MeOH (6 mL) is added morpholine (127 mg, 1.46 mmol, 1.5 eq) at 0° C. The reaction mixture is stirred at room temperature for 16 h. The reaction mixture is filtered and the resulting solid washed with water (3 mL) and dried to give the title compound (200 mg; 80%) as an off white solid. Mass [m/z] 257 [M+H]⁺.

### Step 2: 5-chloro-7-morpholinothiazolo[5,4-d]pyrimidine-2-carbaldehyde

To a stirred solution of 4-(5-chlorothiazolo[5,4-d]pyrimidin-7-yl)morpholine (200 mg, 0.78 mmol, 1 eq.) in THE (5 mL) at −78° C. is added LiHMDS (1.5 mL, 1.56 mmol, 2 eq). The reaction mixture is stirred at same temperature for 30 min. DMF (0.2 mL, 2.35 mmol, 3 eq.) is added and stirred for an additional 2 h. The reaction is quenched with saturated NH₄Cl solution (15 mL) and extracted with ethyl acetate (2×30 mL). Finally, the combined organic layers are washed with water (20 mL), dried over anhydrous Na₂SO₄, filtered, and evaporated under reduced pressure to afford the title compound (210 mg; 94%) as an off while solid. Mass [m/z] 285.1 [M+H]⁺; ¹H-NMR (400 MHz, DMSO-d₆): 9.97 (s, 1H), 4.74 (br s, 2H), 4.14-4.09 (m, 2H), 3.89-3.84 (m, 4H).

### Step 3: (5-chloro-7-morpholinothiazolo[5,4-d]pyrimidin-2-yl)methanol

To a stirred solution of 5-chloro-7-morpholinothiazolo[5,4-d]pyrimidine-2-carbaldehyde (210 mg, 0.74 mmol, 1 eq) in THE (10 mL) is added NaBH₄ (56 mg, 1.48 mmol, 2 eq) at 0° C. The reaction temperature is raised to room temperature and stirred for an additional 5 h. The progress of the reaction is monitored by TLC. After complete consumption of starting material, the reaction mixture is diluted with ethyl acetate (25 mL) and washed with water (10 mL). The organic layer is dried over Na₂SO₄, filtered and concentrated to obtain a brown solid that is triturated with n-pentane to afford the title compound (180 mg; 85%) as a white solid, this is used as such for the next reaction. Mass [m/z] 287.1 [M+H]⁺.

### Step 4: (7-morpholino-5-(3-(m-tolyl)-1H-pyrazol-1-yl)thiazolo[5,4-d]pyrimidin-2-yl)methanol

To a stirred solution of 5-chloro-7-morpholinothiazolo[5,4-d]pyrimidin-2-yl)methanol (180 mg, 0.62 mmol, 1 eq) in 1,4-dioxane:toluene (4:1) (4 mL) is added 3-(m-tolyl)-1H-pyrazole (99 mg, 0.62 mmol, 1 eq), K₃PO₄ (266 mg, 1.25 mmol, 2 eq). The resulting reaction mixture is degassed for 15 minutes under nitrogen, followed by addition of Pd₂(dba)₃ (115 mg, 0.12 mmol, 0.2 eq) and t-Bu-XPhos (106 mg, 0.25 mmol, 0.4 eq) at room temperature. The reaction temperature is raised to 120° C. and stirred for 6 h. After completion, the reaction mixture is cooled to room temperature, filtered through celite pad, and washed with ethyl acetate (10 mL). The filtrate is concentrated to obtain a crude brown gummy liquid which is purified by silica gel chromatography (60-120 mesh) eluting with 5% MeOH/DCM to afford the title compound (110 mg; 43%) as an off-white solid. This is directly taken forward to next step. Mass [m/z] 409.1 [M+H]⁺.

### Step 5: 4-(2-(chloromethyl)-5-(3-(m-tolyl)-1H-pyrazol-1-yl)thiazolo[5,4-d]pyrimidin-7-yl)morpholine

To a stirred solution of (7-morpholino-5-(3-(m-tolyl)-1H-pyrazol-1-yl)thiazolo[5,4-d]pyrimidin-2-yl)methanol (110 mg, 0.27 mmol, 1 eq) in DCM (5 mL) is added SOCl₂ (0.2 mL) at 0° C., the reaction temperature is raised to room temperature and stirred for 2 h. The progress of the reaction is monitored by TLC. After completion, the reaction mixture is concentrated under reduced pressure to obtain a crude solid which is triturated with diethyl ether to afford the title compound (100 mg; 87%) as an off-white solid. Mass [m/z] 427.1 [M+H]⁺. This compound is used to prepare the compounds of Examples 9, 16, 30, 31, 34, and 47.

### Step 6: 4-(2-((4-(methylsulfonyl)piperazin-1-yl)methyl)-5-(3-(m-tolyl)-1H-pyrazol-1-yl)thiazolo[5,4-d]pyrimidin-7-yl)morpholine

To the solution of 4-(2-(chloromethyl)-5-(3-(m-tolyl)-1H-pyrazol-1-yl)thiazolo[5,4-d]pyrimidin-7-yl)morpholine (20 mg, 1 eq) in THE (20 vol) is added 1-(methylsulfonyl)piperazine (8 mg, 2 eq) and K₂CO₃ (3 eq) at room temperature, then the reaction mixture is heated to 80° C. and stirred for 16 h. The reaction mixture is quenched with cold water (10 vol) and extracted with ethyl acetate (2×20 mL). The organic layer is dried over Na₂SO₄, filtered and concentrated to obtain a brown gummy solid which is purified by Prep HPLC (Method-B: GEMINI-C18, 250×21.2 mm, 5 μm; Mobile phase: [ACN: 0.1% of Formic acid in Water]; time/B %: 0/40, 20/65, 21/95), to afford the title compound (2.2 mg; 8%) as an off white solid. Mass [m/z] 555.2 [M+H]⁺; ¹H-NMR (400 MHz, DMSO-d₆): 8.75 (d, J=2.8 Hz, 1H), 7.79 (s, 1H), 7.76 (d, J=7.6 Hz, 1H), 7.36 (t, J=7.6 Hz, 1H), 7.22 (d, J=7.6 Hz, 1H), 7.07 (d, J=2.8 Hz, 1H), 4.35 (br s, 4H), 4.01 (s, 2H), 3.79 (t, J=4.8 Hz, 4H), 3.19 (t, J=4.4 Hz, 4H), 2.93 (s, 3H), 2.68 (br s, 2H), 2.46 (br s, 2H), 2.39 (s, 3H).

### Example 10

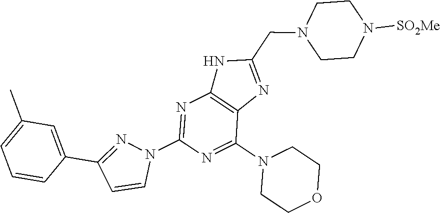 [SMILES: Cc1cccc(-c2ccn(-c3nc(N4CCOCC4)c4nc(CN5CCN(S(C)(=O)=O)CC5)[nH]c4n3)n2)c1]

### Step 1: 4-(2-chloro-9-(tetrahydro-2H-pyran-2-yl)-9H-purin-6-yl)morpholine

To a solution of 2,6-dichloro-9-(tetrahydro-2H-pyran-2-yl)-9H-purine (2 g, 7.35 mmol, 1 eq) in MeOH (20 mL) is added morpholine (959 mg, 11.02 mmol, 1.5 eq) at 0° C. The reaction mixture is brought to room temperature and stirred for 3 h. The progress of the reaction is monitored by TLC. After completion, the reaction mixture is filtered, and the solid washed with water (15 mL) and dried to obtain the title compound (1.2 g; 52%) as an off-white solid. Mass [m/z] 324.2 [M+H]⁺; ¹H-NMR (400 MHz, DMSO-d₆): 7.91 (s, 1H), 5.71 (dd, J=2 Hz, J=10.4 Hz, 1H), 4.29-4.12 (m, 4H), 3.83-3.79 (m, 4H), 3.76-3.73 (m, 1H), 2.1-2.02 (m, 2H), 1.91-1.87 (m, 1H), 1.79-1.61 (m, 3H).

### Step 2: 2-chloro-6-morpholino-9-(tetrahydro-2H-pyran-2-yl)-9H-purine-8-carbaldehyde

To a stirred solution of 4-(2-chloro-9-(tetrahydro-2H-pyran-2-yl)-9H-purin-6-yl) morpholine (1.2 g, 0.78 mmol, 1 eq.) in THE (5 mL) at −78° C. is added LiHMDS (1.5 mL, 1.56 mmol, 2 eq), and the reaction is stirred for 30 min, then DMF (0.2 mL, 2.35 mmol, 3 eq.) is added and the reaction stirred for an additional 2 h at the same temperature. The progress of the reaction is monitored by TLC. After completion of the reaction, it is quenched with saturated NH₄Cl solution (30 mL) and extracted with EtOAc (2×50 mL). Finally, the combined organic layers are washed with water (25 mL) dried over anhydrous Na₂SO₄, filtered, and evaporated under reduced pressure to afford the title compound (900 mg; 69%) as a sticky solid. Mass [m/z] 352.2 [M+H]⁺; ¹H-NMR (400 MHz, DMSO-d₆): 9.95 (s, 1H), 6.22-6.25 (dd, J=2.4, 2.8 Hz, 1H), 4.65 (Br s, 2H), 4.00-4.20 (m, 3H), 3.894 (t, J=4.4 Hz, 4H). 3.70-3.78 (dt, J=2.4, 12 Hz, 1H), 3.2-3.35 (m, 1H), 2.04-2.09 (m, 1H), 1.58-1.87 (m, 6H).

### Step 3: (2-chloro-6-morpholino-9-(tetrahydro-2H-pyran-2-yl)-9H-purin-8-yl)methanol

To a solution of 2-chloro-6-morpholino-9-(tetrahydro-2H-pyran-2-yl)-9H-purine-8-carbaldehyde (1.3 g, 3.70 mmol, 1 eq.) in THE (10 mL) is added NaBH₄ (281 mg, 7.40 mmol, 2 eq) at 0° C. the reaction temperature is raised to RT and stirred for 5 h. The progress of the reaction is monitored by TLC. After complete consumption of starting material, the reaction is diluted with ethyl acetate (50 mL) and the organic layer washed with water (25 mL), dried over Na₂SO₄, filtered and concentrated to obtain a crude off white solid. The crude product is purified by silica gel column (60-120 mesh) eluting with 40-45% EtOAc/hexane to afford the title compound (900 mg; 69%) as an off-white solid. Mass [m/z] 354.1 [M+H]⁺;

### Step 4: 4-(2-chloro-8-(chloromethyl)-9H-purin-6-yl)morpholine

To a solution of (2-chloro-6-morpholino-9-(tetrahydro-2H-pyran-2-yl)-9H-purin-8-yl)methanol (500 mg, 1.41 mmol, 1 eq) in DCM (10 mL) is added SOCl₂ (0.3 mL, 4.23 mmol, 3 eq) at 0° C. then the reaction temperature is raised to 25° C. and stirred for an additional 4 h. The progress of the reaction is monitored by TLC. After completion, the reaction mixture is concentrated under reduced pressure to afford the title compound (520 mg; 52%) as a yellowish solid which is directly taken forward to the next step. Mass [m/z] 288.1 [M+H]⁺.

### Step 5: 4-(2-chloro-8-((4-(methylsulfonyl)piperazin-1-yl)methyl)-9H-purin-6-yl)morpholine

4-(2-chloro-8-(chloromethyl)-9H-purin-6-yl)morpholine (50 mg) is reacted with 1-(methylsulfonyl)piperazine (42 mg, 1.5 eq.) in THF (20 vol) at room temperature, followed by K₂CO₃ (2 eq). The reaction mixture is heated to 70-80° C. for 10-16 h. Progress of the reaction is monitored by TLC. After starting material consumption, the reaction mixture is diluted with water (10 vol) and extracted with EtOAc (2×10 vol). The combined organics are dried over anhydrous Na₂SO₄, filtered and evaporated under reduced pressure to obtain a crude product which is purified by silica gel chromatography eluting with 20% ethyl acetate/hexanes, to provide the title compound (42 mg; 58%) as an off-white solid. Mass [m/z] 416.1 [M+H]⁺; ¹H-NMR (400 MHz, DMSO-d₆): 13.18 (br s, 1H), 4.15 (br s, 4H), 3.72 (m, 7H), 3.12 (t, J=4.4 Hz, 4H), 2.87 (s, 3H).

### Step 6: 4-(8-((4-(methylsulfonyl)piperazin-1-yl)methyl)-2-(3-(m-tolyl)-1H-pyrazol-1-yl)-9H-purin-6-yl)morpholine

4-(2-chloro-8-((4-(methylsulfonyl)piperazin-1-yl)methyl)-9H-purin-6-yl)morpholine (40 mg) and 3-(m-tolyl)-1H-pyrazole (24 mg, 1.5 eq.) are dissolved in a mixture of 1,4-dioxane:toluene: (1:1) (20 vol), to which is added Pd₂(dba)₃ (0.1 eq.), t-Bu-XPhos (0.2 eq.), followed by K₃PO₄ (2 eq.), and the mixture is degassed for 30 min. The resulting reaction mixture is stirred at 120° C. for 6 h, and the reaction monitored by TLC. After complete consumption of the starting material, the reaction mixture is passed through a celite pad and diluted with ethyl acetate (20 vol), and the organics are washed with water (25 vol). Finally, the organic layer is dried over anhydrous Na₂SO₄, filtered, and evaporated under reduced pressure to obtain a crude product. The resulting crude is purified by flash RP column purification using C18, 6 g column eluting with 65% of acetonitrile/water to give the title compound (15 mg; 23%) as an off white solid. Mass [m/z] 538.3 [M+H]⁺; ¹H-NMR (400 MHz, DMSO-d₆): 13.18 (br s, 1H), 8.67 (d, J=2.8 Hz, 1H), 7.76 (s, 1H), 7.71 (d, J=8.0 Hz, 1H), 7.34 (t, J=7.8 Hz, 1H), 7.17 (d, J=7.8 Hz, 1H), 6.98 (d, J=2.8 Hz), 4.27 (br s, 4H), 3.74-3.78 (m, 6H), 3.25 (t, J=9.8 Hz, 4H), 2.88 (s, 3H), 2.66 (m, 4H). 2.49 (s, 3H).

### Example 11

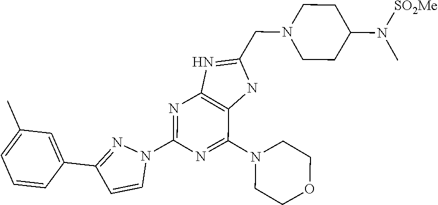 [SMILES: Cc1cccc(-c2ccn(-c3nc(N4CCOCC4)c4[nH]c(CN5CCC(N(C)S(C)(=O)=O)CC5)nc4n3)n2)c1]

### Step 5: N-(1-((2-chloro-6-morpholino-9H-purin-8-yl)methyl)piperidin-4-yl)-N-methylmethanesulfonamide

Following the general procedure of Example 10, 4-(2-chloro-8-(chloromethyl)-9H-purin-6-yl)morpholine (50 mg) is reacted with N-methyl-N-(piperidin-4-yl)methane sulfonamide (50 mg). The resulting crude is purified by silica gel chromatography eluting with 80% ethyl acetate/hexanes, to give the title compound (40 mg; 42%) as an off-white solid. Mass [m/z] 444.9 [M+H]⁺.

### Step 6: N-methyl-N-(1-((6-morpholino-2-(3-(m-tolyl)-1H-pyrazol-1-yl)-9H-purin-8-yl)methyl)piperidin-4-yl)methanesulfonamide

Following the general procedure of Example 10, N-(1-((2-chloro-6-morpholino-9H-purin-8-yl)methyl)piperidin-4-yl)-N-methylmethanesulfonamide (40 mg) is reacted with 3-(m-tolyl)-1H-pyrazole (26 mg). The resulting crude is purified by flash RP column chromatography using C18, 6 g column, eluting with 50% of acetonitrile/water to give the title compound (10 mg; 19%) as an off white solid. Mass [m/z] 566.2 [M+H]⁺; ¹H-NMR (400 MHz, DMSO-d₆): 8.64 (d, J=2.4 Hz, 1H), 7.76 (s, 1H), 7.71 (d, J=7.6 Hz, 1H), 7.34 (t, J=7.6 Hz, 1H), 7.16 (d, J=7.6 Hz, 1H), 6.95 (d, J=2.4 Hz), 4.25 (Br s, 4H), 3.76 (t, J=4.4 Hz, 4H), 3.62 (s 2H), 3.48-3.57 (m, 2H), 2.92 (d, J=12 Hz, 2H). 2.88 (s, 3H), 2.69 (s, 2H), 2.38 (s, 3H), 2.14 (t, J=10.8 Hz, 2H), 1.75 (d, J=7.2 Hz, 2H), 1.56 (d, J=10.4 Hz, 2H).

### Example 12

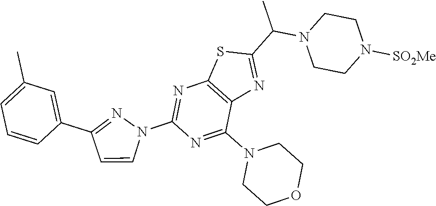 [SMILES: Cc1cccc(-c2ccn(-c3nc(N4CCOCC4)c4nc(C(C)N5CCN(S(C)(=O)=O)CC5)sc4n3)n2)c1]

### Step 1: 5-chloro-7-morpholinothiazolo[5,4-d]pyrimidine-2-carbaldehyde

To a stirred solution of 4-(5-chlorothiazolo[5,4-d]pyrimidin-7-yl)morpholine (400 mg, 1.56 mmol, 1 eq.) in THE (5 mL) at −78° C. is added LiHMDS (3.1 mL, 3.12 mmol, 2 eq). The reaction mixture is stirred at same temperature for 30 min. DMF (0.38 mL, 4.68 mmol, 3 eq.) is added, stirred for another 2 h at the same temperature and the progress of the reaction is monitored by TLC. After completion of the reaction, it is quenched with saturated NH₄Cl solution (15 mL) and extracted with ethyl acetate (2×30 mL). Finally, the combined organic layers are washed with water (20 mL), dried over anhydrous Na₂SO₄, filtered and evaporated under reduced pressure to afford a crude compound as a sticky liquid. Then crude compound is purified through silica gel column (60-120 mesh) eluting at 15-20% of EtOAc/hexane to afford the title compound (230 mg; 52%) as an off-white solid. Mass [m/z] 284.1 [M+H]⁺; ¹H-NMR (400 MHz, DMSO-d₆): 9.97 (s, 1H), 4.76 (br s, 2H), 4.13 (br s, 2H), 3.88 (t, J=4.8 Hz, 4H).

### Step 2: 1-(5-chloro-7-morpholinothiazolo[5,4-d]pyrimidin-2-yl)ethan-1-ol

To a stirred solution of 5-chloro-7-morpholinothiazolo[5,4-d]pyrimidine-2-carbaldehyde (230 mg, 0.80 mmol, 1 eq.) in THE (5 mL) at −78° C. is added methylmagnesium bromide (2.4 mL, 2.42 mmol, 3 eq). Then reaction mixture is brought to room temperature and stirred for an additional 2 h. The progress of the reaction is monitored by TLC. After completion of the reaction, it is quenched with saturated NH₄Cl solution (20 mL) and extracted with ethyl acetate (2×35 mL). Finally, the combined organic layers are washed with water (10 mL) dried over anhydrous Na₂SO₄, filtered, and evaporated under reduced pressure to afford crude compound as a sticky liquid. Then crude compound is purified using silica gel column (60-120 mesh) and product eluting with 25-30% EtOAc/Hexane to afford the title compound (110 mg; 45%) as an off-white solid. Mass [m/z] 301.1 [M+H]⁺; ¹H-NMR (400 MHz, DMSO-d₆): 6.50 (d, J=5.2 Hz, 1H), 5.01-4.95 (m, 1H), 4.24 (br s, 4H), 3.73 (t, J=4.8 4H), 1.49 (d, J=6.8 Hz, 3H).

### Step 3: 1-(7-morpholino-5-(3-(m-tolyl)-1H-pyrazol-1-yl)thiazolo[5,4-d]pyrimidin-2-yl)ethan-1-ol

To a stirred solution of 1-(5-chloro-7-morpholinothiazolo[5,4-d]pyrimidin-2-yl)ethan-1-ol (30 mg, 0.1 mmol, 1 eq.), and 3-(m-tolyl)-1H-pyrazole (24 mg, 0.15 mmoL, 1.5 eq.) dissolved in a mixture of solvents 1,4-dioxane: toluene (1:1) (5 mL) is added Pd₂(dba)₃ (9 mg, 0.01 mmol, 0.1 eq.), and t-Bu-XPhos (8 mg, 0.02 mmol, 0.2 eq.), followed by K₃PO₄ (42 mg, 0.2 mmol, 2 eq.), and the mixture is degassed for 30 min. The reaction mixture is stirred at 120° C. for 6 h. Progress of the reaction is monitored by TLC. After complete consumption of the starting material, the reaction is cooled and filtered through a celite pad to remove catalyst impurities. The filtrate is diluted with EtOAc (20 mL), and the organics are washed with water (8 mL×2), dried over anhydrous Na₂SO₄, filtered, and evaporated under reduced pressure to obtain a crude (35 mg) as light brown solid. The crude compound is purified by silica gel (60-120 mesh) and eluting at 45-50% EtOAc/hexane to afford the title compound (20 mg; 47%) as an off-white solid. Mass [m/z] 423.2 [M+H]⁺.

### Step 4: 4-(2-(1-chloroethyl)-5-(3-(m-tolyl)-1H-pyrazol-1-yl)thiazolo[5,4-d]pyrimidin-7-yl)morpholine

To a stirred solution of 1-(7-morpholino-5-(3-(m-tolyl)-1H-pyrazol-1-yl)thiazolo[5,4-d]pyrimidin-2-yl)ethan-1-ol (20 mg, 0.42 mmol, 1 eq.), in DCM (5 mL) at 0° C. is added SOCl₂ (101 mg, 0.85 mmol, 2 eq). The reaction temperature is allowed to raise to room temperature and stirred for 2 h. The progress of the reaction is monitored by TLC and after completion, it is evaporated under reduced pressure to obtain the title compound (20 mg; 95%) as a yellowish solid. Mass [m/z] 441.1 [M+H]⁺; Step 5: 4-(2-(1-(4-(methylsulfonyl)piperazin-1-yl)ethyl)-5-(3-(m-tolyl)-1H-pyrazol-1-yl)thiazolo[5,4-d]pyrimidin-7-yl)morpholine

To a stirred solution of 4-(2-(1-chloroethyl)-5-(3-(m-tolyl)-1H-pyrazol-1-yl)thiazolo[5,4-d]pyrimidin-7-yl)morpholine (20 mg, 0.07 mmol, 1 eq.) in THE (4 mL) is added 1-(methylsulfonyl)piperazine (18 mg, 0.11 mmol, 1.5 eq) and K₂CO₃ (30 mg, 0.22 mmol, 3 eq) at room temperature. The reaction mixture is heated at reflux for 16 h, and the progress of the reaction is monitored by TLC. After completion of the reaction, it is diluted with ethyl acetate (30 mL) and the organics are washed with water (10 mL), dried over anhydrous Na₂SO₄, filtered, and evaporated under reduced pressure to obtain an off-white solid. Purification of the crude by RP flash (C18-6 g column) eluting with 55-60% of acetonitrile/water affords the title compound (8 mg; 30%) as an off-white solid. Mass [m/z] 569.1 [M+H]⁺; ¹H-NMR (400 MHz, DMSO-d₆): 8.73 (d, J=2.8 Hz, 1H), 7.78 (br s, 1H), 7.75 (d, J=8 Hz, 1H), 7.35 (t, J=7.6 Hz 1H), 7.21 (d, J=7.2 Hz, 1H), 7.05 (d, J=2.4 Hz, 1H), 4.37 (br s, 4H), 4.27-4.24 (m, 1H), 3.79 (t, J=4.4 Hz, 4H), 3.19-3.18 (m, 4H), 2.92 (s, 3H), 2.69-2.67 (m, 4H), 2.39 (s, 3H), 1.44 (d, J=6.8 Hz, 3H).

### Example 13

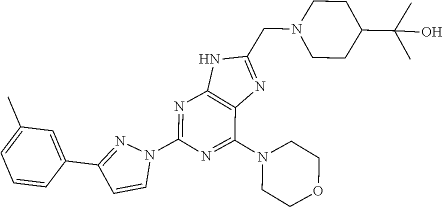 [SMILES: Cc1cccc(-c2ccn(-c3nc(N4CCOCC4)c4nc(CN5CCC(C(C)(C)O)CC5)[nH]c4n3)n2)c1]

### Step 5: 2-(1-((2-chloro-6-morpholino-9H-purin-8-yl)methyl)piperidin-4-yl)propan-2-ol

Following the general procedure of Example 10, 4-(2-chloro-8-(chloromethyl)-9H-purin-6-yl)morpholine (50 mg) is reacted with 2-(piperidin-4-yl)propan-2-ol (37 mg). The resulting crude is purified by silica gel chromatography eluting with 60% ethyl acetate/hexanes, to give the title compound (50 mg; 44%) as an off-white solid. Mass [m/z] 339.9 [M+H]⁺.

### Step 6: 2-(1-((6-morpholino-2-(3-(m-tolyl)-1H-pyrazol-1-yl)-9H-purin-8-yl)methyl) piperidin-4-yl)propan-2-ol

Following the general procedure of Example 10, 2-(1-((2-chloro-6-morpholino-9H-purin-8-yl)methyl)piperidin-4-yl)propan-2-ol (40 mg) is reacted with 3-(m-tolyl)-1H-pyrazole (24 mg). The resulting crude is purified by flash RP column chromatography using C18, 6 g column, product eluting with 70% of acetonitrile/water to give the title compound (8 mg; 15%) as an off white solid. Mass [m/z] 517.3 [M+H]⁺; ¹H-NMR (400 MHz, DMSO-d₆): 13.18 (br s, 1H), 8.66 (d, J=2.4 Hz, 1H), 7.76 (s, 1H), 7.71 (d, J=7.6 Hz, 1H), 7.34 (t, J=7.6 Hz, 1H), 7.17 (d, J=7.6 Hz, 1H), 6.98 (d, J=2.4 Hz), 4.27 (Br s, 4H), 4.05 (s, 1H), 3.77 (t, J=4.8 Hz, 4H), 3.62 (s 2H), 2.86 (Br s, 2H), 2.69 (s, 2H), 2.38 (s, 3H), 2.08 (br s, 2H), 1.63 (d, J=9.6 Hz, 2H), 1.29-1.35 (m, 2H). 1.15 (d, J=6.0 Hz, 1H), 1.02 (s, 6H).

### Example 14

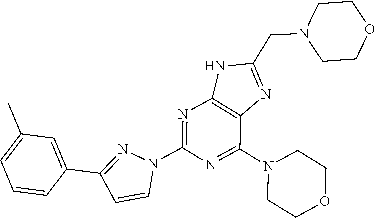 [SMILES: Cc1cccc(-c2ccn(-c3nc(N4CCOCC4)c4nc(CN5CCOCC5)[nH]c4n3)n2)c1]

### Step 5: 4-((2-chloro-6-morpholino-9H-purin-8-yl)methyl)morpholine

Following the general procedure of Example 10, 4-(2-chloro-8-(chloromethyl)-9H-purin-6-yl)morpholine (50 mg) is reacted with 22.5 mg of morpholine (22.5 mg). The resulting crude product is purified by silica gel chromatography eluting with 20% ethyl acetate/hexanes to give the title compound (26 mg; 45%) as an off-white solid. Mass [m/z] 339.9 [M+H]⁺

### Step 6: 4-((6-morpholino-2-(3-(m-tolyl)-1H-pyrazol-1-yl)-9H-purin-8-yl)methyl) morpholine

Following the general procedure of Example 10, 4-((2-chloro-6-morpholino-9H-purin-8-yl)methyl)morpholine (30 mg) is reacted with 3-(m-tolyl)-1H-pyrazole (14 mg). The resulting crude is purified by flash RP column chromatography using C18, 6 g column, product eluting with 75% of acetonitrile/water to give the title compound (3 mg; 8%) as an off white solid. Mass [m/z] 459.1 [M+H]⁺; ¹H-NMR (400 MHz, DMSO-d₆): 13.20 (br s, 1H), 8.66 (d, J=2.8 Hz, 1H), 7.76 (s, 1H), 7.71 (d, J=8.0 Hz, 1H), 7.34 (t, J=7.8 Hz, 1H), 7.17 (d, J=7.8 Hz, 1H), 6.98 (d, J=2.8 Hz), 4.23 (Br s, 4H), 3.77 (t, J=4.4 Hz, 4H), 3.66 (s, 2H), 3.60 (t, J=4.4 Hz, 4H), 2.45 (m, 4H). 2.38 (s, 3H).

### Example 15

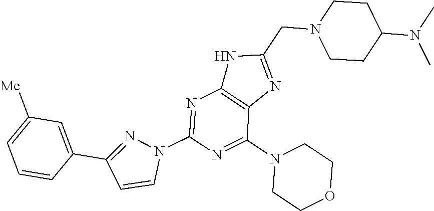 [SMILES: Cc1cccc(-c2ccn(-c3nc(N4CCOCC4)c4nc(CN5CCC(N(C)C)CC5)[nH]c4n3)n2)c1]

### Step 4: (6-morpholino-9-(tetrahydro-2H-pyran-2-yl)-2-(3-(m-tolyl)-1H-pyrazol-1-yl)-9H-purin-8-yl)methanol

Following the first three steps of the procedure according to Example 10, to a solution of (2-chloro-6-morpholino-9-(tetrahydro-2H-pyran-2-yl)-9H-purin-8-yl)methanol (500 mg, 1.41 mmol, 1 eq) in 1,4-dioxane: toluene (4:1) (5 mL) is added 3-(m-tolyl)-1H-pyrazole (268 mg, 1.69 mmol, 1.2 eq), K₃PO₄ (600 mg, 2.83 mmol, 2 eq). The resulting reaction mixture is degassed for 10 minutes under nitrogen, followed by the addition of Pd₂(dba)₃ (129 mg, 0.141 mmol, 0.2 eq) and t-Bu-XPhos (120 mg, 0.283 mmol, 0.4 eq) at room temperature. The reaction temperature is then raised to 120° C. and mixture stirred for 6 h.

After completion, the reaction mixture is cooled to room temperature, filtered through a celite pad and washed with ethyl acetate (10 mL) and the filtrate is concentrated to obtain a crude brown gummy liquid. The crude product is purified by silica gel column (60-120 mesh) eluting with 2-5% MeOH/DCM to obtain the title compound (350 mg; 52%) as an off-white solid. Mass [m/z] 476.3 [M+H]⁺; ¹H-NMR (400 MHz, DMSO-d₆): 8.59 (d, J=2.8 Hz, 1H), 7.85 (br s, 1H), 7.76 (d, J=7.6 Hz, 1H), 7.32 (t, J=7.6 Hz, 1H), 7.17 (d, J=7.2 Hz, 1H), 6.77 (d, J=2.8 Hz, 1H), 6.1-6.07 (m, 1H), 5.0 (d, J=14 Hz, 1H), 4.86 (d, J=14.4 Hz, 1H), 4.37 (br s, 3H), 4.21-4.20 (m, 1H), 3.90-3.84 (m, 5H), 3.48 (br s, 1H), 2.42 (s, 3H), 2.07-2.04 (m, 3H), 1.92-1.7 (m, 3H).

### Step 5: 4-(8-(chloromethyl)-2-(3-(m-tolyl)-1H-pyrazol-1-yl)-9H-purin-6-yl) morpholine

To a solution of (6-morpholino-9-(tetrahydro-2H-pyran-2-yl)-2-(3-(m-tolyl)-1H-pyrazol-1-yl)-9H-purin-8-yl)methanol (350 mg, 0.73 mmol, 1 eq) in DCM (5 mL) is added SOCl₂ (0.2 mL) at 0° C. and then the reaction temperature is raised to 25° C. and stirred for an additional 2 h. The progress of the reaction is monitored by TLC. After completion, the reaction mixture is concentrated under reduced pressure to afford the title compound (290 mg; 96%) as a yellowish solid. Mass [m/z] 410.1 [M+H]⁺; ¹H-NMR (400 MHz, DMSO-d₆): 13.58 (Br s, 1H), 8.69 (d, J=2.8 Hz, 1H), 7.77 (Br s, 1H), 7.74 (d, J=7.6 Hz, 1H), 7.35 (t, J=7.6 Hz, 1H), 7.2 (d, J=7.2 Hz, 1H), 7.01 (d, J=2.8 Hz, 1H), 4.88 (s, 2H), 4.28 (t, J=7.6, 1H), 3.78 (t, J=7.0 Hz, 1H), 2.39 (s, 3H).

### Step 6: N,N-dimethyl-1-((6-morpholino-2-(3-(m-tolyl)-1H-pyrazol-1-yl)-9H-purin-8-yl)methyl)piperidin-4-amine

To a solution of 4-(8-(chloromethyl)-9-methyl-2-(3-(m-tolyl)-1H-pyrazol-1-yl)-9H-purin-6-yl)morpholine (30 mg, 1 eq.) in THE (3 mL) is added N,N-dimethylpiperidin-4-amine (17 mg, 2 eq.) and K₂CO₃ (3 eq) at room temperature, then the reaction mixture is heated to 80° C. and stirred for 16 h. The reaction mixture is quenched with cold water (20 vol) and extracted with ethyl acetate (20 vol×2). The organic layer is dried over Na₂SO₄, filtered and concentrated to obtain a crude which is purified by Prep HPLC (Method-B: Kinetix, EVO, C18, 250×21.2 mm, 5 μm; Mobile phase: [ACN: 0.1% of Formic acid in Water]; time/B %: 0/20, 15/55, 20/70, 21/95) eluting with 65-70% ACN/water to afford the title compound (8 mg); 22%) as a white solid. Mass [m/z] 502.4 [M+H]⁺; ¹H-NMR (400 MHz, DMSO-d₆): 8.67 (d, J=2.8 Hz, 1H), 8.23 (s, 1H), 7.77 (s, 1H), 7.71 (d, J=7.6 Hz, 1H), 7.34 (t, J=7.2 Hz, 1H), 7.17 (d, J=7.6 Hz, 1H), 7.00 (d, J=2.4 Hz, 1H), 4.27 (br s, 4H), 3.77 (t, J=4.4 Hz, 4H), 3.68 (s, 3H), 2.84 (d, J=12 Hz, 2H), 2.33 (s, 4H), 2.18 (s, 6H), 2.04 (t, J=11.6 Hz, 3H), 1.70 (d, J=12.4 Hz, 2H), 1.42 (m, 2H).

### Example 16

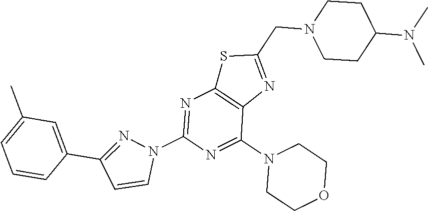 [SMILES: Cc1cccc(-c2ccn(-c3nc(N4CCOCC4)c4nc(CN5CCC(N(C)C)CC5)sc4n3)n2)c1]

### Step 6: N,N-dimethyl-1-((7-morpholino-5-(3-(m-tolyl)-1H-pyrazol-1-yl)thiazolo[5,4-d]pyrimidin-2-yl)methyl)piperidin-4-amine

Following the general procedure of Example 9, 4-(2-(chloromethyl)-5-(3-(m-tolyl)-1H-pyrazol-1-yl)thiazolo[5,4-d]pyrimidin-7-yl)morpholine (40 mg) is reacted with N,N-dimethylpiperidin-4-amine (24 mg) and the resulting crude is purified by Prep HPLC (Method-B: GEMINI-C18, 250×21.2 mm, 5 μm; Mobile phase: [ACN: 0.1% of Formic acid in Water]; time/B %: 0/25, 10/55, 20/95), to afford the title compound (10 mg; 10%) as an off-white solid. Mass [m/z] 519.1 [M+H]⁺; ¹H-NMR (400 MHz, DMSO-d₆): 8.73 (d, J=2.8 Hz, 1H), 7.79 (s, 1H), 7.75 (d, J=8.0 Hz, 1H), 7.35 (t, J=7.6 Hz, 1H), 7.21 (d, J=7.6 Hz, 1H), 7.05 (d, J=2.4 Hz, 1H), 4.35 (br s, 4H), 3.88 (s, 2H), 3.78 (t, J=4.4 Hz, 1H), 2.98 (d, J=11.6 Hz, 1H), 2.39 (s, 3H), 2.08 (s, 6H), 1.77 (d, J=12.4 Hz, 2H), 1.45-1.43 (m, 2H), 1.23 (s, 1H).

### Example 17

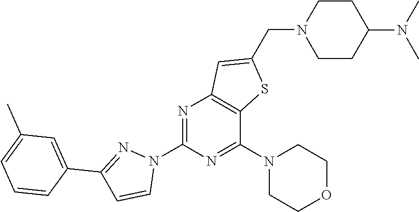 [SMILES: Cc1cccc(-c2ccn(-c3nc(N4CCOCC4)c4sc(CN5CCC(N(C)C)CC5)cc4n3)n2)c1]

### Step 1: 2-chloro-4-morpholinothieno[3,2-d]pyrimidine-6-carbaldehyde

To a stirred solution of 4-(2-chlorothieno[3,2-d]pyrimidin-4-yl)morpholine (200 mg, 0.78 mmol, 1 eq.) in THE (5 mL) at −78° C. is added LiHMDS (1.5 mL, 1.56 mmol, 2 eq). The reaction mixture is stirred at same temperature for 30 min and then DMF (0.2 mL, 2.35 mmol, 3 eq.) is added at same temperature for 2 h. The progress of the reaction is monitored by TLC. After completion of the reaction, it is quenched with saturated NH₄Cl solution (15 mL) and extracted with EtOAc (2×30 mL). Finally, the combined organic layer is washed with water (20 mL) dried over anhydrous Na₂SO₄, filtered and evaporated under reduced pressure to afford a crude compound as a sticky liquid. The crude product is purified by silica gel column chromatography (60-120 mesh) eluting at 15-20% EtOAc/hexane, and the product fractions are evaporated to afford the title compound (120 mg, 54%) as an off-white solid. Mass [m/z] 284.1 [M+H]⁺; ¹H-NMR (400 MHz, DMSO-d₆): 10.17 (s, 1H), 7.97 (s, 1H), 4.06 (t, J=4.08 Hz, 4H), 3.86 (t, 5.0 Hz, 4H).

### Step 2: 1-(2-chloro-4-morpholinothieno[3,2-d]pyrimidin-6-yl)ethan-1-ol

To a stirred solution of 2-chloro-4-morpholinothieno[3,2-d]pyrimidine-6-carbaldehyde (350 mg, 1.23 mmol, 1 eq.) in THE (5 mL) at −78° C. is added methylmagnesium bromide (3.7 mL, 3.71 mmol, 3 eq). The reaction mixture is stirred at room temperature for 2 h, and the progress of the reaction is monitored by TLC. After completion of the reaction, it is quenched with saturated NH₄Cl solution (15 mL) and extracted with EtOAc (2×30 mL). Finally, the combined organic layers are washed with water (10 mL) dried over anhydrous Na₂SO₄, filtered, and evaporated under reduced pressure to afford the crude compound as a sticky liquid. The crude is purified by silica gel column chromatography (60-120 mesh) eluting at 20-25% EtOAc/hexane to afford the title compound (250 mg; 68%) as an off-white solid. Mass [m/z] 300.1 [M+H]⁺; ¹H-NMR (400 MHz, DMSO-d₆): 7.164 (s, 1H), 5.27-5.21 (m, 1H), 4.01-3.98 (m, 4H), 3.85-3.82 (m, 4H), 2.26 (d, J=4.8 Hz, 1H), 1.66 (d, J=6.4 Hz, 3H).

### Step 3: 1-(4-morpholino-2-(3-(m-tolyl)-1H-pyrazol-1-yl)thieno[3,2-d]pyrimidin-6-yl)ethan-1-ol

To a stirred solution of 1-(2-chloro-4-morpholinothieno[3,2-d]pyrimidin-6-yl)ethan-1-ol (250 mg, 0.83 mmol, 1 eq.) and 3-(m-tolyl)-1H-pyrazole (198 mg, 1.25 mmoL, 1.5 eq.) dissolved in a mixture of toluene: 1,4-dioxane (1:1) (5 mL) is added Pd₂(dba)₃ (77 mg, 0.083 mmol, 0.1 eq.), and t-Bu-XPhos (71 mg, 0.16 mmol, 0.2 eq.), followed by K₃PO₄ (353 mg, 0.19 mmol, 2 eq.), and the mixture is degassed for 30 min. The reaction mixture is stirred at 120° C. for 6 h. Progress of the reaction is monitored by TLC. After complete consumption of the starting material, the catalyst is filtered and the filtrate is diluted with EtOAc (30 mL), and washed with water (10 mL). Finally, the organic layer is dried over anhydrous Na₂SO₄, filtered and evaporated under reduced pressure to obtain crude product (260 mg) as a light brown solid. The crude is purified by silica gel column chromatography (60-120 mesh) eluting at 40% EtOAc/hexane, to afford the title compound (180 mg; 51%) as an off white solid. Mass [m/z] 422.1 [M+H]⁺; Step 4: 4-(6-(1-chloroethyl)-2-(3-(m-tolyl)-1H-pyrazol-1-yl)thieno[3,2-d]pyrimidin-4-yl)morpholine

To a stirred solution of 1-(4-morpholino-2-(3-(m-tolyl)-1H-pyrazol-1-yl)thieno[3,2-d]pyrimidin-6-yl)ethan-1-ol (180 mg, 0.42 mmol, 1 eq.), in DCM (5 mL) at 0° C. is added SOCl₂ (101 mg, 0.85 mmol, 2 eq). The reaction temperature is raised to room temperature and stirred for 2 h. The progress of the reaction is monitored by TLC and after completion, it is evaporated under reduced pressure to obtain the title compound (180 mg; 95%) as a white solid. Mass [m/z] 440.2 [M+H]⁺; ¹H-NMR (400 MHz, DMSO-d₆): 8.76 (d, J=2.8 Hz, 1H), 7.8 (Brs, 1H), 7.76 (d, J=8 Hz, 1H), 7.64 (s, 1H), 7.35 (t, J=7.6 Hz 1H), 7.2 (d, J=7.2 Hz, 1H), 7.02 (d, J=2.4 Hz, 1H), 5.85-5.83 (m, 1H), 4.04-4.02 (m, 4H), 3.81-3.78 (m, 4H), 2.39 (s, 3H), 1.96 (d, J=6.8 Hz, 3H).

### Step 5: N,N-dimethyl-1-(1-(4-morpholino-2-(3-(m-tolyl)-1H-pyrazol-1-yl)thieno[3,2-d]pyrimidin-6-yl)ethyl)piperidin-4-amine

To a solution of 4-(6-(1-chloroethyl)-2-(3-(m-tolyl)-1H-pyrazol-1-yl)thieno[3,2-d]pyrimidin-4-yl)morpholine (50 mg, 1 eq.) in 1,4-dioxane (0.5 mL) is added N,N-dimethylpiperidin-4-amine (22 mg, 1.5 eq.) at room temperature and stirred at 150° C. for 3 h in a microwave vial. After completion, the reaction mixture is quenched with cold water (5 mL) and extracted with ethyl acetate (2×15 mL). The organic layer is dried over Na₂SO₄ filtered and concentrated to obtain a crude off white solid (58 mg) which is purified by Prep HPLC (Method-B: Kinetex, EVO, C18, 250×21.2 mm, 5 μm; Mobile phase: [ACN: 0.1% of Formic acid in Water]; time/B %: 0/10, 12/35, 15/60, 20/95), which after lyophilization affords the title compound (15 mg; 25%) as an off-white solid.

Mass [m/z] 440.2 [M+H]⁺; ¹H-NMR (400 MHz, DMSO-d₆): 9.70 (br s, 1H), 8.74 (d, J=2.8 Hz, 1H), 7.79 (br s, 1H), 7.75 (d, J=8 Hz, 1H), 7.64 (s, 1H), 7.36 (t, J=7.6 Hz 1H), 7.21 (d, J=7.2 Hz, 1H), 7.05 (d, J=2.4 Hz, 1H), 4.99-4.97 (m, 1H), 4.01-4.10 (m, 4H), 3.91-3.85 (m, 4H), 3.75-3.5 (m, 2H), 3.31-3.40 (m, 1H), 2.95-2.92 (m, 2H), 2.76 (s, 6H), 2.39 (s, 3H), 2.27-2.17 (m, 2H), 1.93-1.81 (m, 2H), 1.77 (d, J=6.8 Hz, 3H).

### Example 18

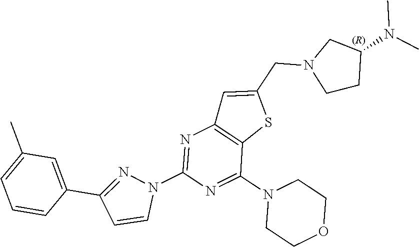

### Step 1: 2-chloro-4-morpholinothieno[3,2-d]pyrimidine-6-carbaldehyde

To a stirred solution of 4-(2-chlorothieno[3,2-d]pyrimidin-4-yl)morpholine (200 mg, 0.78 mmol, 1 eq.) in THE (5 mL) at −78° C. is added LiHMDS (1.5 mL, 1.56 mmol, 2 eq). The reaction mixture is stirred at the same temperature for 30 min, then DMF (0.2 mL, 2.35 mmol, 3 eq.) is added and stirred for another 2 hours at the same temperature. The progress of the reaction is monitored by TLC, after completion of the reaction, it is quenched with saturated NH₄Cl solution (15 mL) and extracted with ethyl acetate (2×30 mL). Finally, the combined organic layers are washed with water (20 mL) and dried over anhydrous Na₂SO₄, filtered and evaporated under reduced pressure to afford crude compound as a sticky liquid. The crude compound is purified by silica gel (60-120 mesh) column chromatography, eluting at 15-20% of EtOAc/hexane to afford the title compound (120 mg; 120 mg; 54%) as an off-white solid. Mass [m/z] 284.1 [M+H]⁺; ¹H-NMR (400 MHz, DMSO-d₆): 10.17 (s, 1H), 7.97 (s, 1H), 4.06 (t, J=4.08 Hz, 4H), 3.86 (t, 5.0 Hz, 4H).

### Step 2: (2-chloro-4-morpholinothieno[3,2-d]pyrimidin-6-yl)methanol

To a stirred solution of 2-chloro-4-morpholinothieno[3,2-d]pyrimidine-6-carbaldehyde (1.5 g, 5.3 mmol, 1 eq.) in MeOH (20 mL) at 0° C. is added NaBH₄ (402 mg, 10.6 mmol, 2 eq). The reaction mixture is allowed to reach room temperature and stirred for 4 h. The progress of the reaction is monitored by TLC. After completion of the reaction, it is quenched with saturated NH₄Cl solution (30 mL) and extracted with EtOAc (2×50 mL). Finally, the combined organic layers are washed with water (20 mL) dried over anhydrous Na₂SO₄, filtered, and evaporated under reduced pressure to afford crude compound as a sticky liquid. The crude compound is triturated with diethyl ether (2×10 mL) to afford the title compound (1.1 g; 73%) as a light brown solid. Mass [m/z] 286 [M+H]⁺; ¹H-NMR (400 MHz, DMSO-d₆): 7.20 (t, J=1.2 Hz, 1H), 4.98 (d, J=4.8 Hz, 2H), 4.0-3.98 (m, 4H), 3.85-3.82 (m, 4H), 2.32 (br s, 1H).

### Step 3: (4-morpholino-2-(3-(m-tolyl)-1H-pyrazol-1-yl)thieno[3,2-d]pyrimidin-6-yl)methanol

To a stirred solution of (2-chloro-4-morpholinothieno[3,2-d]pyrimidin-6-yl)methanol (34-6) (1.1 g, 0.28 mmol, 1 eq.), and 3-(m-tolyl)-1H-pyrazole (550 mg, 1.5 eq.) in a mixture of toluene:dioxane (1:1) (3 mL) is added Pd₂(dba)₃ (640 mg, 0.028 mmol, 0.1 eq.), and t-Bu-XPhos (600 mg, 0.056 mmol, 0.2 eq.), followed by K₃PO₄ (1.5 g, 0.56 mmol, 2 eq.), and the reaction is degassed for 30 min. The reaction mixture is stirred at 120° C. for 6 h. Progress of the reaction is monitored by TLC. After completion of the reaction, the catalyst is filtered and the filtrate is diluted with EtOAc (100 mL), and washed with water (30 mL). Finally, the organic layer is dried over anhydrous Na₂SO₄, filtered and evaporated under reduced pressure to obtain a crude (1.4 g) light brown solid. The crude is purified by flash RP using water/ACN as eluting at 55-60% ACN/water followed by lyophilization to afford the title compound (350 mg; 22%) as an off white solid. Mass [m/z] 408.2 [M+H]⁺; ¹H-NMR (400 MHz, DMSO-d₆): 8.73 (d, J=2.8 Hz, 1H), 7.79 (br s, 1H), 7.75 (d, J=8 Hz, 1H), 7.35 (t, J=7.6 Hz 1H), 7.2 (d, J=7.2 Hz, 1H), 7.02 (d, J=2.4 Hz, 1H), 5.94 (t, J=5.6, 1H), 4.85-4.83 (m, 2H), 4.03-4.0 (m, 4H), 3.81-3.79 (m, 4H), 2.39 (s, 3H).

### Step 4: 4-(6-(chloromethyl)-2-(3-(m-tolyl)-1H-pyrazol-1-yl)thieno[3,2-d]pyrimidin-4-yl)morpholine

To a stirred solution of (4-morpholino-2-(3-(m-tolyl)-1H-pyrazol-1-yl)thieno[3,2-d]pyrimidin-6-yl)methanol (200 mg, 0.49 mmol, 1 eq.), in DCM (5 mL) at 0° C. is added SOCl₂ (116 mg, 0.98 mmol, 2 eq). The reaction is allowed to warm to room temperature and it is then stirred for 2 h. The progress of the reaction is monitored by TLC and after completion, it is evaporated under reduced pressure to afford the title compound (200 mg; 96%) as a white solid. Mass [m/z] 426.1 [M+H]⁺; ¹H-NMR (400 MHz, DMSO-d₆): 8.65 (s, 1H), 8.47 (s, 1H), 7.95 (m, 2H), 7.38 (t, J=7.6 Hz, 1H), 7.20 (d, J=7.2 Hz, 1H), 6.86 (d, J=2.4 Hz, 1H), 4.18 (brs, 4H), 3.94 (brs, 4H), 2.44 (s, 3H).

### Step 5: (R)—N,N-dimethyl-1-((4-morpholino-2-(3-(m-tolyl)-1H-pyrazol-1-yl)thieno[3,2-d]pyrimidin-6-yl)methyl)pyrrolidin-3-amine

To a stirred solution of 4-(6-(chloromethyl)-2-(3-(m-tolyl)-1H-pyrazol-1-yl)thieno[3,2-d]pyrimidin-4-yl)morpholine (50 mg, 1 eq) in THE (20 vol) at room temperature is added (R)—N,N-dimethylpyrrolidin-3-amine (26.6 mg) followed by K₂CO₃ (3 eq). The reaction mixture is heated to 70-80° C. for 8 to 12 h. Progress of the reaction is monitored by TLC. After starting material consumption, the reaction mixture is diluted with water (10 vol) and extracted with EtOAc (2×10 vol). Combined organic fractions are dried over anhydrous Na₂SO₄, filtered, and evaporated under reduced pressure to provide a crude product (65 mg) as an off white solid. The crude compound is purified by Prep HPLC (Method-B: Kinetex, EVO, C18, 250×21.2 mm, 5 μm; Mobile phase: [ACN: 0.1% of Formic acid in Water]; time/B %: 0/10, 12/35, 15/60, 20/95), and after lyophilization affords the title compound (10 mg; 17%) as an off-white solid. Mass [m/z] 504.3 [M+H]⁺; ¹H-NMR (400 MHz, DMSO-d₆): 8.72 (d, J=2.8 Hz, 1H), 7.79 (Brs, 1H), 7.75 (d, J=8 Hz, 1H), 7.40 (s, 1H), 7.35 (t, J=7.6 Hz 1H), 7.2 (d, J=7.2 Hz, 1H), 7.02 (d, J=2.4 Hz, 1H), 4.01-3.97 (m, 4H), 3.88 (s, 2H), 3.81-3.78 (m, 4H), 2.81-2.55 (m, 4H), 2.43 (Brs, 1H), 2.39 (s, 3H), 2.12 (s, 6H), 1.85-1.95 (m, 1H), 1.7-1.66 (m, 1H).

### Example 19

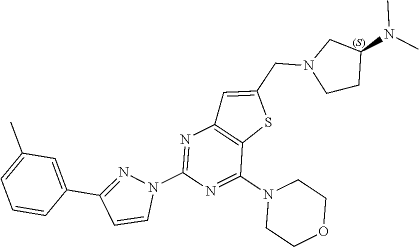

### Step 5: (S)—N,N-dimethyl-1-((4-morpholino-2-(3-(m-tolyl)-1H-pyrazol-1-yl)thieno[3,2-d]pyrimidin-6-yl)methyl)pyrrolidin-3-amine

Following the general procedure of Example 18, 4-(6-(chloromethyl)-2-(3-(m-tolyl)-1H-pyrazol-1-yl)thieno[3,2-d]pyrimidin-4-yl)morpholine (50 mg) is treated with (S)—N,N-dimethylpyrrolidin-3-amine (26.6 mg) to obtain crude (62 mg) as an off white solid. The crude compound is purified by Prep HPLC (Method-B: Kinetex, EVO, C18, 250×21.2 mm, 5 μm; Mobile phase: [ACN: 0.1% of Formic acid in Water]; time/B %: 0/10, 12/35, 15/60, 20/95), and after lyophilization affords the title product (12 mg; 20%) as an off-white solid. Mass [m/z] 504.3 [M+H]⁺; ¹H-NMR (400 MHz, DMSO-d₆): 8.72 (d, J=2.8 Hz, 1H), 7.79 (Brs, 1H), 7.75 (d, J=8 Hz, 1H), 7.38 (s, 1H), 7.35 (t, J=7.6 Hz 1H), 7.2 (d, J=7.2 Hz, 1H), 7.02 (d, J=2.4 Hz, 1H), 4.01-3.97 (m, 4H), 3.88 (s, 2H), 3.81-3.78 (m, 4H), 2.81-2.58 (m, 4H), 2.44-2.41 (m, 2H), 2.39 (s, 3H), 2.11 (s, 6H), 1.85-1.95 (m, 1H), 1.7-1.66 (m, 1H).

### Example 20

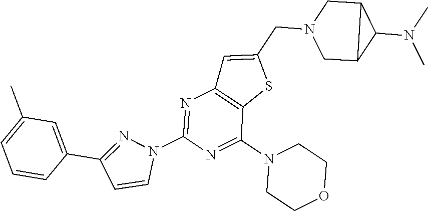 [SMILES: Cc1cccc(-c2ccn(-c3nc(N4CCOCC4)c4sc(CN5CC6C(C5)C6N(C)C)cc4n3)n2)c1]

### Step 5: N,N-dimethyl-3-((4-morpholino-2-(3-(m-tolyl)-1H-pyrazol-1-yl)thieno[3,2-d]pyrimidin-6-yl)methyl)-3-azabicyclo[3.1.0]hexan-6-amine

Following the general procedure of Example 18, 4-(6-(chloromethyl)-2-(3-(m-tolyl)-1H-pyrazol-1-yl)thieno[3,2-d]pyrimidin-4-yl)morpholine (50 mg) is treated with N,N-dimethyl-3-azabicyclo[3.1.0]hexan-6-amine (29.6 mg) to obtain crude (69 mg) as an off white solid. The crude compound is purified by Prep HPLC (Method-B: Kinetex, EVO, C18, 250×21.2 mm, 5 μm; Mobile phase: [ACN: 0.1% of Formic acid in Water]; time/B %: 0/10, 10/30, 15/55, 18/95), and after lyophilization affords the title compound (8 mg; 14%) as off-white solid. Mass [m/z] 516.1 [M+H]⁺; ¹H-NMR (400 MHz, DMSO-d₆): 8.72 (d, J=2.8 Hz, 1H), 7.78 (Brs, 1H), 7.75 (d, J=8 Hz, 1H), 7.38 (s, 1H), 7.35 (t, J=7.6 Hz 1H), 7.2 (d, J=7.2 Hz, 1H), 7.02 (d, J=2.4 Hz, 1H), 4.01-3.99 (m, 4H), 3.92 (s, 2H), 3.81-3.79 (m, 4H), 3.33-2.3 (m, 2H), 2.39 (s, 3H), 2.36-2.32 (m, 2H), 2.22 (s, 4H), 2.19 (s, 2H), 1.66 (br s, 2H).

### Example 21

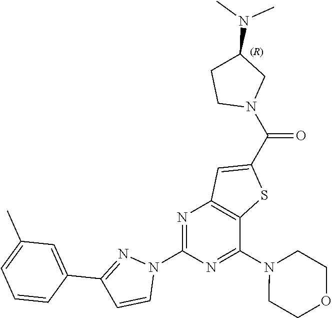

### Step 1: 2-chloro-4-morpholinothieno[3,2-d]pyrimidine-6-carboxylic acid

To a stirred solution of 4-(2-chlorothieno[3,2-d]pyrimidin-4-yl)morpholine (200 mg, 0.78 mmol, 1 eq.) in THE (5 mL) at −78° C. is added LiHMDS (1.5 mL, 1.56 mmol, 2 eq). The reaction mixture is allowed to warm to room temperature and is stirred for 1 h and the progress of the reaction is monitored by TLC. After completion, the reaction is cooled to 0° C., dry ice is added and the reaction is stirred for 1 h. The reaction is quenched with saturated NH₄Cl solution (10 mL) and extracted with EtOAc (2×30 mL). The organic layer is dried over anhydrous Na₂SO₄, filtered, and evaporated under reduced pressure to afford the title compound (180 mg, 76%) as an off-white solid. Mass [m/z] 300.1 [M+H]⁺; ¹H-NMR (400 MHz, DMSO-d₆): 7.25 (s, 1H), 3.90 (t, J=4.4 Hz, 4H), 3.74 (t, J=5.2 Hz, 4H).

### Step 2: (R)-(2-chloro-4-morpholinothieno[3,2-d]pyrimidin-6-yl)(3-(dimethylamino) pyrrolidin-1-yl)methanone

To a stirred solution of 2-chloro-4-morpholinothieno[3,2-d]pyrimidine-6-carboxylic acid (40 mg, 1 eq.) in DMF (10 vol) is added (R)—N,N-dimethylpyrrolidin-3-amine (23 mg), HATU (1.5 eq.) and DIPEA (3.0 eq.). The reaction is stirred at room temperature for 18 h and the progress of the reaction is monitored by TLC. After completion, it is diluted with ethyl acetate (20 vol), washed with ice cold water (2×15 vol) and brine solution (20 vol). Finally, the organic layers are dried over Na₂SO₄, filtered, and evaporated under vacuum to obtain a crude product which is purified by silica gel chromatography with 60% EtOAc/hexane gave the title compound (50 mg; 96%) as an off-white solid. Mass [m/z] 396.2 [M+H]⁺.

### Step 3: (R)-(3-(dimethylamino)pyrrolidin-1-yl)(4-morpholino-2-(3-(m-tolyl)-1H-pyrazol-1-yl)thieno[3,2-d]pyrimidin-6-yl)methanone

To a stirred solution of (R)-(2-chloro-4-morpholinothieno[3,2-d]pyrimidin-6-yl)(3-(dimethylamino)pyrrolidin-1-yl)methanone (40 mg, 1 eq.), and 3-(m-tolyl)-1H-pyrazole (24 mg, 1.5 eq.) dissolved in mixture of toluene: 1,4-dioxane (1:1) (3 mL) is added Pd₂(dba)₃ (0.1 eq.), and t-Bu-XPhos (0.2 eq.), followed by K₃PO₄ (2 eq.), and the mixture is degassed for 30 min. The reaction is heated at 120° C. for 6 h and progress of the reaction is monitored by TLC. After complete consumption of the starting material, the reaction mixture is passed through a Celite pad and diluted with ethyl acetate (20 vol), and the organics are washed with water (15 vol), dried over anhydrous Na₂SO₄, filtered and evaporated under reduced pressure. The resulting crude solid is purified by Prep HPLC (Method-B: GEMINI-C18, 250×21.2 mm, 5 μm; Mobile phase: [ACN: 0.1% of Formic acid in Water]; time/B %: 0/25, 10/65, 15/95) to afford the title compound (10 mg; 19%) as an off white solid. Mass [m/z] 518.2 [M+H]⁺; ¹H-NMR (400 MHz, DMSO-d₆): 8.73 (d, J=2.8 Hz, 1H), 7.79 (br s, 1H), 7.75 (d, J=8 Hz, 1H), 7.35 (t, J=7.6 Hz 1H), 7.2 (d, J=7.2 Hz, 1H), 7.02 (d, J=2.4 Hz, 1H), 5.94 (t, J=5.6, 1H), 4.85-4.83 (m, 2H), 4.03-4.0 (m, 4H), 3.81-3.79 (m, 4H), 2.39 (s, 3H).

### Example 22

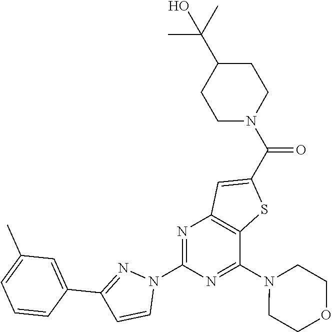 [SMILES: Cc1cccc(-c2ccn(-c3nc(N4CCOCC4)c4sc(C(=O)N5CCC(C(C)(C)O)CC5)cc4n3)n2)c1]

### Step 2: (2-chloro-4-morpholinothieno[3,2-d]pyrimidin-6-yl)(4-(2-hydroxypropan-2-yl)piperidin-1-yl)methanone

Following the general procedure of Example 21, 2-chloro-4-morpholinothieno[3,2-d]pyrimidine-6-carboxylic acid (40 mg) is reacted with 2-(piperidin-4-yl)propan-2-ol (28 mg) to provide the crude (49 mg) as a sticky liquid. The crude is purified by silica gel column chromatography eluting at 60% EtOAc/hexane to afford the title compound (40 mg; 71%) as an off-white solid. Mass [m/z] 425 [M+H]⁺.

### Step 3: (4-(2-hydroxypropan-2-yl)piperidin-1-yl)(4-morpholino-2-(3-(m-tolyl)-1H-pyrazol-1-yl)thieno[3,2-d]pyrimidin-6-yl)methanone

Following the general procedure of Example 21, (2-chloro-4-morpholinothieno[3,2-d]pyrimidin-6-yl)(4-(2-hydroxypropan-2-yl)piperidin-1-yl)methanone (40 mg) is reacted with 3-(m-tolyl)-1H-pyrazole (22 mg). The resulting crude is purified by Prep HPLC (Method-B: GEMINI-C18, 250×21.2 mm, 5 μm; Mobile phase: [ACN: 0.1% of Formic acid in Water]; time/B %: 0/45, 10/65, 25/95) to afford the title compound (10 mg; 19%) as an off white solid. Mass [m/z] 547.3 [M+H]⁺; ¹H-NMR (400 MHz, DMSO-d₆): 8.76 (d, J=2.8 Hz, 1H), 7.80 (br s, 1H), 7.76 (d, J=8 Hz, 1H), 7.35 (t, J=11 Hz 1H), 7.21 (d, J=6.8 Hz, 1H), 7.02 (d, J=2.8 Hz, 1H), 4.55 (t, J=5.6, 1H), 4.19 (s, 1H), 4.05-4.31 (m, 2H), 3.83-3.81 (m, 4H), 3.5-3.4 (m, 4H), 2.49 (s, 3H), 1.85-1.75 (m, 2H), 1.2-1.3 (br s, 3H), 1.07 (s, 6H).

### Example 23

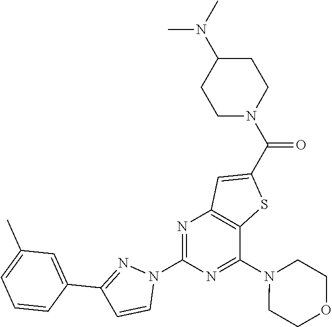 [SMILES: Cc1cccc(-c2ccn(-c3nc(N4CCOCC4)c4sc(C(=O)N5CCC(N(C)C)CC5)cc4n3)n2)c1]

### Step 2: (2-chloro-4-morpholinothieno[3,2-d]pyrimidin-6-yl)(4-(dimethylamino)piperidin-1-yl)methanone

Following the general procedure of Example 21, 2-chloro-4-morpholinothieno[3,2-d]pyrimidine-6-carboxylic acid (50 mg) is reacted with N,N-dimethylpiperidin-4-amine (32 mg). The crude is purified by silica gel chromatography eluting with 70% EtOAc/hexane to afford the title compound (35 mg; 51%) as an off-white solid. Mass [m/z] 382.1 [M+H]⁺; ¹H-NMR (400 MHz, DMSO-d₆): 7.41 (s, 1H), 4.01 (t, J=4.8 Hz, 4H), 3.85 (t, J=5.2 Hz, 4H), 2.28 (s, 6H), 1.93 (br s, 2H), 1.25 (br s, 7H), 0.92-0.82 (m, 4H).

### Step 3: (4-(dimethylamino)piperidin-1-yl)(4-morpholino-2-(3-(m-tolyl)-1H-pyrazol-1-yl)thieno[3,2-d]pyrimidin-6-yl)methanone

Following the general procedure of Example 21, (2-chloro-4-morpholinothieno[3,2-d]pyrimidin-6-yl)(4-(dimethylamino)piperidin-1-yl)methanone (35 mg) is reacted with 3-(m-tolyl)-1H-pyrazole (20 mg). The resulting crude is purified by Prep HPLC (Method-B: Kinetix, EVO, C18, 250×21.2 mm, 5 μm; Mobile phase: [ACN: 0.1% of Formic acid in Water]; time/B %: 0/20, 15/55, 20/70, 21/95) to afford the title product (5 mg; 11%) as an off white solid. Mass [m/z] 547.3 [M+H]⁺; ¹H-NMR (400 MHz, DMSO-d₆): 8.76 (d, J=2.8 Hz, 1H), 7.8 (br s, 1H), 7.77-7.72 (m, 2H), 7.36 (t, J=7.6 Hz 1H), 7.2 (d, J=7.2 Hz, 1H), 7.05 (d, J=2.4 Hz, 1H), 4.03 (t, J=4.6 Hz, 1H), 3.81 (t, J=4.4 Hz, 4H), 2.49 (s, 4H), 2.22 (s, 6H), 1.81-1.86 (m, 2H).

### Example 24

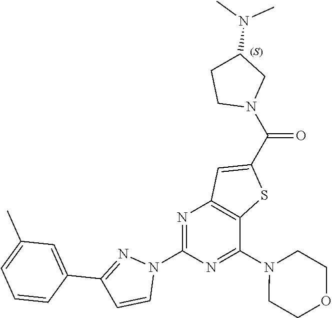

### Step 2: (S)-(2-chloro-4-morpholinothieno[3,2-d]pyrimidin-6-yl)(3-(dimethylamino)pyrrolidin-1-yl)methanone

Following the general procedure of Example 21, 2-chloro-4-morpholinothieno[3,2-d]pyrimidine-6-carboxylic acid (40 mg) is reacted with (S)—N,N-dimethylpyrrolidin-3-amine (23 mg) which after silica gel chromatography eluting with 60% EtOAc/hexane affords the title compound (45 mg; 86%) as an off-white solid. Mass [m/z] 396.2 [M+H]⁺.

### Step 3: (S)-(3-(dimethylamino)pyrrolidin-1-yl)(4-morpholino-2-(3-(m-tolyl)-1H-pyrazol-1-yl)thieno[3,2-d]pyrimidin-6-yl)methanone

Following the general procedure of Example 21, (S)-(2-chloro-4-morpholinothieno[3,2-d]pyrimidin-6-yl)(3-(dimethylamino)pyrrolidin-1-yl)methanone (40 mg) is reacted with 3-(m-tolyl)-1H-pyrazole (22 mg). The resulting crude is purified by Prep HPLC (Method-B: Kinetix, C18, 250×21.2 mm, 5 μm; Mobile phase: [ACN: 0.1% of Formic acid in Water]; time/B %: 0/25, 20/95) to afford the title compound (10 mg; 19%) as an off white solid. Mass [m/z] 518.3 [M+H]⁺; ¹H-NMR (400 MHz, DMSO-d₆): 8.75 (s, 1H), 7.79 (br s, 1H), 7.75 (d, J=7.6 Hz, 1H), 7.36 (t, J=11.4 Hz 1H), 7.2 (d, J=7.6 Hz, 1H), 7.05 (d, J=2.8 Hz, 1H), 4.06 (t, J=7 Hz 4H), 3.829 (t, J=4.8 Hz, 4H), 2.39 (s, 3H) 2.33-2.32 (m, 4H), 1.23 (s, 1H).

### Example 25

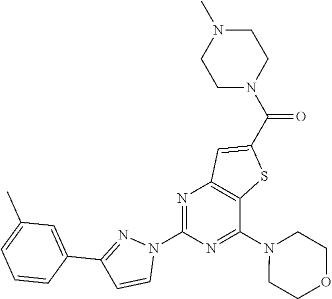 [SMILES: Cc1cccc(-c2ccn(-c3nc(N4CCOCC4)c4sc(C(=O)N5CCN(C)CC5)cc4n3)n2)c1]

### Step 2: (2-chloro-4-morpholinothieno[3,2-d]pyrimidin-6-yl)(4-methylpiperazin-1-yl)methanone

Following the general procedure of Example 21, 2-chloro-4-morpholinothieno[3,2-d]pyrimidine-6-carboxylic acid (50 mg) is reacted with 1-methylpiperazine (25 mg) which after purification by silica gel chromatography eluting with 45% EtOAc/hexane affords the title compound (50 mg; 79%) as an off-white solid. Mass [m/z] 382.1 [M+H]⁺.

### Step 3: (4-methylpiperazin-1-yl)(4-morpholino-2-(3-(m-tolyl)-1H-pyrazol-1-yl)thieno[3,2-d]pyrimidin-6-yl)methanone

Following the general procedure of Example 21, (2-chloro-4-morpholinothieno[3,2-d]pyrimidin-6-yl)(4-methylpiperazin-1-yl)methanone (50 mg) is reacted with 3-(m-tolyl)-1H-pyrazole (31 mg). The resulting crude is purified by Prep HPLC (Method-B: KINETIX EVO, 250×21.2 mm, 5 μm; Mobile phase: [ACN: 0.1% of formic acid in Water]; time/B %: 0/45, 10/65, 25/95) to afford the title compound (15 mg; 22%) as an off white solid. Mass [m/z] 504.2 [M+H]⁺; ¹H-NMR (400 MHz, DMSO-d₆): 8.75 (d, J=2.8 Hz, 1H), 7.79 (br s, 1H), 7.77-7-73 (m, 2H), 7.36 (t, J=7.6 Hz 1H), 7.2 (d, J=7.2 Hz, 1H), 7.05 (d, J=2.4 Hz, 1H), 4.03 (t, J=4.8, 4H), 3.81 (t, J=4.8 Hz, 4H), 3.67 (t, J=4.8 Hz, 4H), 2.39 (br s, 7H), 2.22 (s, 3H).

### Example 26

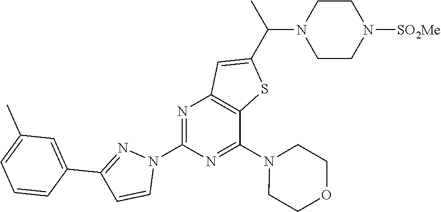 [SMILES: Cc1cccc(-c2ccn(-c3nc(N4CCOCC4)c4sc(C(C)N5CCN(S(C)(=O)=O)CC5)cc4n3)n2)c1]

### Step 5: 4-(6-(1-(4-(methylsulfonyl)piperazin-1-yl)ethyl)-2-(3-(m-tolyl)-1H-pyrazol-1-yl)thieno[3,2-d]pyrimidin-4-yl)morpholine

Following the general procedure of Example 17, 4-(6-(1-chloroethyl)-2-(3-(m-tolyl)-1H-pyrazol-1-yl)thieno[3,2-d]pyrimidin-4-yl)morpholine is treated with 1-(methylsulfonyl) piperazine (28 mg) to obtain a crude (55 mg) which is purified by Flash RP chromatography (C18, 6 g column) using water/acetonitrile as solvent system. The product is eluting at 50-55% acetonitrile/water and after evaporation affords the title compound (2 mg; 3%) as a white solid. Mass [m/z] 568.3 [M+H]⁺; ¹H-NMR (400 MHz, DMSO-d₆): 8.72 (d, J=2.8 Hz, 1H), 7.78 (br s, 1H), 7.75 (d, J=8 Hz, 1H), 7.41 (s, 1H), 7.35 (t, J=7.6 Hz 1H), 7.20 (d, J=7.2 Hz, 1H), 7.02 (d, J=2.4 Hz, 1H), 4.18-4.17 (m, 1H), 3.4.05-3.95 (m, 4H), 3.81-3.72 (m, 4H), 3.19-3.10 (m, 4H), 2.9 (s, 3H), 2.65-2.57 (m, 4H), 2.39 (s, 3H), 1.46 (d, J=6.8 Hz, 3H)

### Example 27

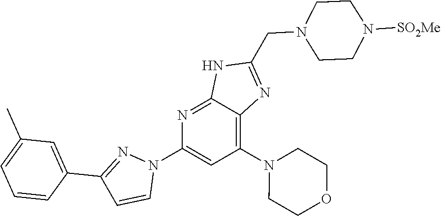 [SMILES: Cc1cccc(-c2ccn(-c3cc(N4CCOCC4)c4nc(CN5CCN(S(C)(=O)=O)CC5)[nH]c4n3)n2)c1]

### Step 1: 4,6-dichloro-3-nitropyridin-2-amine

To a stirred solution of 4,6-dichloropyridin-2-amine (3 g, 18.5 mmol, 1 eq.) at 0° C. is added H₂SO₄ (16 mL), and the mixture is stirred at the same temperature for 30 min. Then HNO₃ (1 mL) is added dropwise at 0° C. The reaction is stirred at the same temperature for 5 h. Progress of the reaction is monitored by TLC. After completion of the reaction, it is quenched with ice-cold water, the obtained solid is filtered to provide crude (2.8 g) as yellow solid. This crude is purified by silica gel (60-120 mesh) column chromatography using 12% EtOAc in hexane as an eluent to afford the title compound (1.2 g; 31%) as a yellow solid. Mass [m/z] 208.1 [M+H]⁺; ¹H-NMR (400 MHz, DMSO-d₆): 6.83 (s, 1H), 6.29 (brs, 2H).

### Step 2: 4,6-dichloropyridine-2,3-diamine

To a stirred solution of 4,6-dichloro-3-nitropyridin-2-amine (1.2 g, 5.79 mmol, 1 eq.), dissolved in IPA (36 mL), is added iron (1.6 g, 28.9 mmol, 5 eq.), then the reaction mixture is cooled to 0° C., 6N HCL (6 mL) is added, and the mixture is stirred at room temperature for 3 h. Progress of the reaction is monitored by TLC. After completion of the reaction, the crude is Celite filtered, and the filtrate is quenched with saturated NaHCO₃ solution and extracted with EtOAc (80 mL). The organic layers are washed with water (20 mL) and dried over anhydrous Na₂SO₄, filtered, and evaporated under reduced pressure to provide a crude (980 mg) as brown solid. This crude is purified by using silica gel (60-120 mesh) column chromatography using 45% EtOAc in hexane as an eluent to afford the title compound (750 mg; 75%) as a brown solid. Mass [m/z] 170.0 [M+H]⁺; ¹H-NMR (400 MHz, CDCl₃): 6.756 (s, 1H), 4.46 (brs, 2H), 3.57 (brs, 2H).

### Step 3: 2-((benzyloxy)methyl)-5,7-dichloro-3H-imidazo[4,5-b]pyridine

To a stirred solution of 4,6-dichloropyridine-2,3-diamine (700 mg, 3.95 mmol, 1 eq.), 2-(benzyloxy)acetic acid (1.3 g, 7.91 mmol, 2 eq.) is added, and the neat reaction is stirred at 150° C. for 4 h. Progress of the reaction is monitored by TLC. After completion of the reaction, it is diluted with EtOAc (55 mL). The organic layer is washed with aqueous NaHCO₃ (20 mL) first then washed with water (15 mL). Finally, the organic layers are dried over anhydrous Na₂SO₄ filtered and evaporated under reduced pressure to obtain crude (1.1 g) as brown liquid. Crude is purified by using silica gel (60-120 mesh) column chromatography using 25% EtOAc in Hexane as an eluent to afford the title compound (750 mg; 62%) as an ash color solid. Mass [m/z] 308.1 [M+H]⁺; ¹H-NMR (400 MHz, DMSO-d₆): 13.75 (brs, 1H), 7.56 (s, 1H), 7.41-7-29 (m, 5H), 4.77 (s, 2H), 4.62 (s, 1H).

### Step 4: 2-((benzyloxy)methyl)-5,7-dichloro-3-(tetrahydro-2H-pyran-2-yl)-3H-imidazo[4,5-b]pyridine

To a stirred solution of 2-((benzyloxy)methyl)-5,7-dichloro-3H-imidazo[4,5-b]pyridine (500 mg, 1.62 mmol, 1 eq.) in DCM (10 mL) is added pyridinium p-toluenesulfonate (PPTS) (41 mg, 0.16 mmol, 0.1 eq) followed by 3,4-dihydropyran (DHP) (0.5 mL, 6.49 mmol, 4 eq). The reaction mixture is stirred at room temperature for 18 h. Progress of the reaction is monitored by TLC. After completion of the reaction, it is diluted with EtOAc (60 mL). The separated organic layer is washed with aqueous NaHCO₃ (20 mL) followed by water (15 mL). Finally, the organic layers are dried over anhydrous Na₂SO₄, filtered, and evaporated under reduced pressure to provide a crude (550 mg) as colorless liquid. The crude is purified by silica gel (60-120) column chromatography using 20% EtOAc in hexane as an eluent to afford the title compound (450 mg; 71%) as an off-white solid. Mass [m/z] 392.2 [M+H]⁺; ¹H-NMR (400 MHz, DMSO-d₆): 7.36-7.26 (m, 6H), 5.03 (d, J=12.4 Hz, 1H), 4.86 (d, J=12.4 Hz 1H), 4.67-4.60 (m, 2H), 4.13-4.09 (m, 1H), 3.64-3.63 (m, 1H), 2.62-2.70 (m, 1H), 1.63-2.13 (m, 6H).

### Step 5: 4-(2-((benzyloxy)methyl)-5-chloro-3-(tetrahydro-2H-pyran-2-yl)-3H-imidazo[4,5-b]pyridin-7-yl)morpholine

To a stirred solution of 2-((benzyloxy)methyl)-5,7-dichloro-3-(tetrahydro-2H-pyran-2-yl)-3H-imidazo[4,5-b]pyridine (200 mg, 0.51 mmol, 1 eq.) in methanol (5 mL) is added morpholine (2 mL, 1 vol). The reaction mixture is stirred at 80° C. for 12 h. Progress of the reaction is monitored by TLC. After completion of the reaction, it is diluted with EtOAc (35 mL). The separated organic layer is washed with 1 N HCl (15 mL), and washed with water (15 mL). Finally, the organic layer is dried over anhydrous Na₂SO₄ and evaporated under reduced pressure to obtain crude (230 mg) as colorless liquid. Crude is purified by using silica gel (60-120) column chromatography using 15-20% EtOAc in hexane as an eluent to afford the title compound (130 mg; 58%) as a light-yellow solid. Mass [m/z]443.2 [M+H]⁺; ¹H-NMR (400 MHz, DMSO-d₆): 7.40-7.29 (m, 5H), 6.59 (s, 1H), 5.69-5.66 (dd, J=2 Hz, J=2.4 Hz, 1H), 4.88 (d, 1H), 4.69 (d, 1H), 4.58 (d, J=1.2 Hz, 2H), 4.03-4.0 (m, 1H), 3.91-3.89 (m, 4H), 3.75-3.73 (m, 4H), 3.6-3.58 (m, 1H), 2.56-2.51 (m, 1H), 1.90-1.89 (m, 1H), 1.76-1.72 (m, 1H), 1.50 (brs, 1H). 1.26 (s, 2H).

### Step 6: 4-(2-((benzyloxy)methyl)-3-(tetrahydro-2H-pyran-2-yl)-5-(3-(m-tolyl)-1H-pyrazol-1-yl)-3H-imidazo[4,5-b]pyridin-7-yl)morpholine

A solution of t-butyl-XPhos (57 mg, 0.13 mmol, 0.2 eq), and Pd₂(dba)₃ (62 mg, 0.067 mmol, 0.1 eq) in toluene (3 mL) is degassed for 10 min and heated to 100° C. for 5 min until the solution turns clear, then this mixture at 100° C. is added slowly to a degassed mixture of 4-(2-((benzyloxy)methyl)-5-chloro-3-(tetrahydro-2H-pyran-2-yl)-3H-imidazo[4,5-b]pyridin-7-yl)morpholine (300 mg, 0.67 mmol, 1 eq.), 3-(m-tolyl)-1H-pyrazole (160 mg, 1.01 mmol, 1.5 eq), and K₃PO₄ (430 mg, 1.35 mmol, 2 eq) in 1,4-dioxane (5 mL) taken in another flask (at 100° C.). The resulting reaction mixture is heated to 120° C. and stirred for 5 h. The progress of the reaction is monitored by TLC and after completion it is diluted with EtOAc (50 mL) and washed with water (20 mL). Finally, the organic layer is dried over anhydrous Na₂SO₄, filtered, and evaporated under reduced pressure to provide crude (400 mg) as a brownish gummy material. The crude compound is purified by RP flash purification (C18-6 g column) eluting with 55% acetonitrile/water, to afford the title compound (180 mg; 47%) as an off-white solid; Mass [m/z] 563.3 [M+H]⁺; ¹H-NMR (400 MHz, DMSO-d₆): 8.59 (d, J=2.4 Hz, 1H), 7.77 (s, 1H), 7.73 (d, J=8 Hz, 1H), 7.4-7.3 (m, 8H), 7.17 (d, J=7.6 Hz 1H), 6.75 (d, J=2.4 Hz, 1H), 5.83 (dd, J=2.4 Hz, J=11.2 Hz, 1H), 4.94 (d, J=12.4 Hz, 1H), 4.78 (d, J=12 Hz, 1H), 4.63 (d, J=2.8 Hz, 1H), 4.16-4.13 (m, 1H), 4.03-4.01 (m, 4H), 3.94-3.92 (m, 4H), 3.66-3.65 (m, 1H), 2.75-2.85 (m, 1H), 2.43 (s, 3H), 2.05 (Brs, 1H), 1.91 (d, J=13.6 Hz, 1H), 1.71 (t, J=8.8 Hz, 2H).

### Step 7: (7-morpholino-3-(tetrahydro-2H-pyran-2-yl)-5-(3-(m-tolyl)-1H-pyrazol-1-yl)-3H-imidazo[4,5-b]pyridin-2-yl)methanol

To a stirred solution of 4-(2-((benzyloxy)methyl)-3-(tetrahydro-2H-pyran-2-yl)-5-(3-(m-tolyl)-1H-pyrazol-1-yl)-3H-imidazo[4,5-b]pyridin-7-yl)morpholine (150 mg, 0.51 mmol, 1 eq.) in EtOAc (3 mL) is added 10% Pd/C (50% wet) (120 mg). The reaction mixture is stirred at room temperature for 18 h under hydrogen atmosphere (30 psi). Progress of the reaction is monitored by TLC. After completion of the reaction, it is diluted with EtOAc (35 mL) and filtered under nitrogen atmosphere though Celite pad. The filtrate is evaporated under reduced pressure to provide a crude (100 mg) as colorless liquid. The crude is purified by silica gel (60-120) column chromatography with 45% EtOAc in hexane as an eluent to afford the title compound (80 mg; 63%) as an off white solid. Mass [m/z] 475.3 [M+H]⁺; ¹H-NMR (400 MHz, DMSO-d₆): 8.58 (d, J=2.8 Hz, 1H), 7.77 (s, 1H), 7.73 (d, J=8 Hz, 1H), 7.35-7.31 (t, J=7.6 Hz, 1H), 7.18 (d, J=7.6 Hz 1H), 6.76 (d, J=2.4 Hz, 1H), 5.96 (dd, J=2.4 Hz, J=11.2 Hz, 1H), 5.0 (d, J=12.4 Hz, 1H), 4.89 (m, 1H), 4.27 (d, J=11.6 Hz, 1H), 4.03-4.00 (m, 4H), 3.95-3.92 (m, 4H), 3.81-3.79 (m, 1H), 3.45-3.42 (m, 1H), 2.43 (s, 3H), 1.82-1.76 (m, 3H).

### Step 8: 4-(2-(chloromethyl)-5-(3-(m-tolyl)-1H-pyrazol-1-yl)-3H-imidazo[4,5-b]pyridin-7-yl)morpholine

To a stirred solution of (7-morpholino-3-(tetrahydro-2H-pyran-2-yl)-5-(3-(m-tolyl)-1H-pyrazol-1-yl)-3H-imidazo[4,5-b]pyridin-2-yl)methanol (280 mg, 0.59 mmol, 1 eq.), in DCM (6 mL) at 0° C. is added SOCl₂ (0.2 mL). The reaction mass is allowed to warm to room temperature and it is stirred for 2 h. The progress of the reaction is monitored by TLC. After starting material consumption, it is evaporated under reduced pressure to afford the title compound (220 mg; 91%) as a white solid. Mass [m/z] 409.1 [M+H]⁺; ¹H-NMR (400 MHz, DMSO-d₆): 8.79 (brs, 1H), 7.73 (d, J=8 Hz, 2H), 7.35 (t, J=7.6 Hz, 1H), 7.26 (d, J=7.6 Hz 1H), 6.87 (brs, 1H), 4.75 (s, 2H), 4.25 (brs, 4H), 3.95 (brs, 4H), 2.45 (s, 3H).

### Step 9: 4-(2-((4-(methylsulfonyl)piperazin-1-yl)methyl)-5-(3-(m-tolyl)-1H-pyrazol-1-yl)-3H-imidazo[4,5-b]pyridin-7-yl)morpholine

To a stirred solution of 4-(2-(chloromethyl)-5-(3-(m-tolyl)-1H-pyrazol-1-yl)-3H-imidazo[4,5-b]pyridin-7-yl)morpholine (30 mg, 1 eq) in THE (20 vol) at room temperature is added 1-(methylsulfonyl)piperazine (1.5 eq) followed by K₂CO₃ (3 eq). The reaction mixture is heated to 70-80° C. for 8 to 12 h. Progress of the reaction is monitored by TLC. After starting material consumption, the reaction mixture is diluted with water (10 vol) and extracted twice with EtOAc (2×10 vol). Combined organic fractions are dried over anhydrous Na₂SO₄, filtered, and evaporated under reduced pressure to obtain crude product (45 mg) which is purified by RP flash column chromatography (C18-6 g) eluting at 52% acetonitrile/water to afford the title compound (15 mg; 38%) as an off white solid. Mass [m/z] 409.1 [M+H]⁺; ¹H-NMR (400 MHz, DMSO-d₆): 12.78 (Brs, 1H), 8.52 (d, J=2.8 Hz, 1H), 7.78 (Brs, 2H), 7.36-7.33 (t, J=7.6 Hz, 1H), 7.18 (d, J=7.6 Hz 1H), 7.00 (d, J=2.4 Hz, 1H), 3.94 (Brs, 4H), 3.83-3.82 (m, 4H), 3.72 (s, 2H), 3.15 (t, J=4.4 Hz, 4H), 2.88 (s, 3H), 2.67 (Brs, 4H), 2.39 (s, 3H).

### Example 28

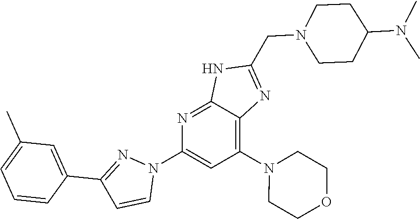 [SMILES: Cc1cccc(-c2ccn(-c3cc(N4CCOCC4)c4nc(CN5CCC(N(C)C)CC5)[nH]c4n3)n2)c1]

### Step 9: N,N-dimethyl-1-((7-morpholino-5-(3-(m-tolyl)-1H-pyrazol-1-yl)-3H-imidazo[4,5-b]pyridin-2-yl)methyl)piperidin-4-amine

Following the general procedure of Example 27, 4-(2-(chloromethyl)-5-(3-(m-tolyl)-1H-pyrazol-1-yl)-3H-imidazo[4,5-b]pyridin-7-yl)morpholine (40 mg) is reacted with N,N-dimethylpiperidine-4-amine (1.5 eq.) to provide a crude product (50 mg) which is purified by RP flash column chromatography (C18-6 g), eluting with 65% of acetonitrile/water to afford the title compound (18 mg; 37%) as an off white solid. Mass [m/z] 409.1 [M+H]⁺; ¹H-NMR (400 MHz, DMSO-d₆): 12.85 (Brs, 1H), 8.51 (d, J=2.4 Hz, 1H), 7.77 (Brs, 2H), 7.35 (t, J=7.6 Hz, 1H), 7.19 (d, J=7.6 Hz 1H), 7.01 (d, J=2.4 Hz, 1H), 3.94 (Brs, 4H), 3.83-3.82 (m, 4H), 3.68 (s, 2H), 2.98 (d, J=11.6 Hz 1H), 2.63 (Brs, 6H), 2.39 (s, 3H), 2.12 (t, J=11.6 Hz, 2H), 1.92 (d, J=10.4 Hz, 2H), 1.61 (m, 2H).

### Example 29

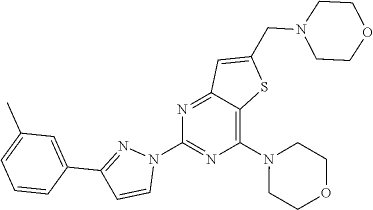 [SMILES: Cc1cccc(-c2ccn(-c3nc(N4CCOCC4)c4sc(CN5CCOCC5)cc4n3)n2)c1]

### Step 9: 4-((4-morpholino-2-(3-(m-tolyl)-1H-pyrazol-1-yl)thieno[3,2-d]pyrimidin-6-yl)methyl)morpholine

Following the general procedure of Example 18, 4-(6-(chloromethyl)-2-(3-(m-tolyl)-1H-pyrazol-1-yl)thieno[3,2-d]pyrimidin-4-yl)morpholine (50 mg) is treated with morpholine (20 mg) to obtain crude (58 mg) as an off white solid. The crude compound is purified by flash RP chromatography, eluting with 70-75% acetonitrile/water. Product fractions are lyophilized to afford the title compound (15 mg; 27%) as an off-white solid. Mass [m/z]477.3 [M+H]⁺; ¹H-NMR (400 MHz, DMSO-d₆): 8.72 (d, J=2.8 Hz, 1H), 7.78 (br s, 1H), 7.75 (d, J=8 Hz, 1H), 7.42 (s, 1H), 7.35 (t, J=7.6 Hz 1H), 7.2 (d, J=7.2 Hz, 1H), 7.02 (d, J=2.4 Hz, 1H), 4.01 (t, J=4.4 Hz, 4H), 3.87 (s, 2H), 3.81 (t, J=4.8 Hz, 4H), 3.62 (t, J=4.4 Hz, 4H), 2.39 (s, 3H).

### Example 30

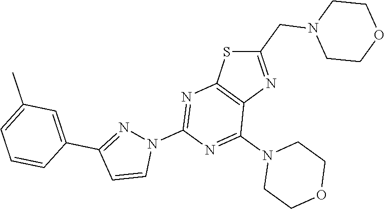 [SMILES: Cc1cccc(-c2ccn(-c3nc(N4CCOCC4)c4nc(CN5CCOCC5)sc4n3)n2)c1]

### Step 6: 4-((7-morpholino-5-(3-(m-tolyl)-1H-pyrazolyl)thiazolo[5,4-d]pyrimidin-2-yl)methyl)morpholine

Following the general procedure of Example 9, 4-(2-(chloromethyl)-5-(3-(m-tolyl)-1H-pyrazol-1-yl)thiazolo[5,4-d]pyrimidin-7-yl)morpholine (60 mg) is reacted with morpholine (24 mg) and the resulting crude (68 mg) is purified by Flash RP chromatography (C18, 6 g column) eluting with 70-75% ACN/water to afford the title compound (11 mg; 16%) as a white solid. Mass [m/z] 478.3 [M+H]⁺; ¹H-NMR (400 MHz, DMSO-d₆): 8.73 (d, J=2.8 Hz, 1H), 7.79 (s, 1H), 7.75 (d, J=7.6 Hz, 1H), 7.35 (t, J=7.6 Hz, 1H), 7.21 (d, J=7.6 Hz, 1H), 7.05 (d, J=2.8 Hz, 1H), 4.35 (Brs, 4H), 3.92 (s, 2H), 3.79 (t, J=4.8 Hz, 4H), 3.64 (t, J=4.4 Hz, 4H), 2.57 (t, J=4 Hz, 4H), 2.39 (s, 3H).

### Example 31

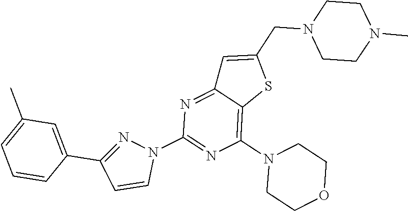 [SMILES: Cc1cccc(-c2ccn(-c3nc(N4CCOCC4)c4sc(CN5CCN(C)CC5)cc4n3)n2)c1]

### Step 5: 4-(6-((4-methylpiperazin-1-yl)methyl)-2-(3-(m-tolyl)-1H-pyrazol-1-yl)thieno[3,2-d]pyrimidin-4-yl)morpholine

Following the general procedure of Example 18, 4-(6-(chloromethyl)-2-(3-(m-tolyl)-1H-pyrazol-1-yl)thieno[3,2-d]pyrimidin-4-yl)morpholine (50 mg) is treated with 1-methylpiperazine (24 mg) to obtain crude (56 mg) as an off white solid. The crude compound is purified by flash RP, eluting with 68% acetonitrile/water which after lyophilization affords the title compound (15 mg; 26%) as an off-white solid. Mass [m/z] 490.3 [M+H]⁺; ¹H-NMR (400 MHz, DMSO-d₆): 8.72 (d, J=2.8 Hz, 1H), 7.79 (br s, 1H), 7.75 (d, J=8 Hz, 1H), 7.40 (s, 1H), 7.35 (t, J=7.6 Hz 1H), 7.2 (d, J=7.2 Hz, 1H), 7.02 (d, J=2.4 Hz, 1H), 4.01-3.99 (m, 4H), 3.86 (s, 2H), 3.81-3.79 (m, 4H), 2.39 (s, 3H), 2.19 (br s, 3H), 1.23 (s, 2H).

### Example 32

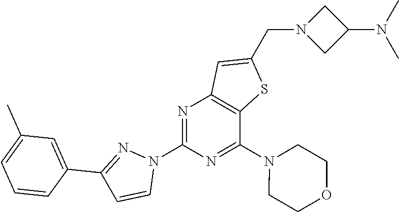 [SMILES: Cc1cccc(-c2ccn(-c3nc(N4CCOCC4)c4sc(CN5CC(N(C)C)C5)cc4n3)n2)c1]

### Step 5: N,N-dimethyl-1-((4-morpholino-2-(3-(m-tolyl)-1H-pyrazol-1-yl)thieno[3,2-d]pyrimidin-6-yl)methyl)azetidin-3-amine

Following the general procedure of Example 18, 4-(6-(chloromethyl)-2-(3-(m-tolyl)-1H-pyrazol-1-yl)thieno[3,2-d]pyrimidin-4-yl)morpholine (50 mg) is treated with N,N-dimethyl-azetidine-3-amine (23.5 mg) to obtain crude (54 mg) as an off-white solid. The crude compound is purified by flash RP, eluting with 50-55% acetonitrile/water which after lyophilization affords the title compound (11 mg; 19%) as an off-white solid. Mass [m/z] 490.2 [M+H]⁺; ¹H-NMR (400 MHz, DMSO-d₆): 8.72 (d, J=2.8 Hz, 1H), 7.79 (Brs, 1H), 7.75 (d, J=8 Hz, 1H), 7.38 (s, 1H), 7.35 (t, J=7.6 Hz 1H), 7.2 (d, J=7.2 Hz, 1H), 7.02 (d, J=2.4 Hz, 1H), 4.01-3.99 (m, 4H), 3.95 (s, 2H), 3.81-3.78 (m, 4H), 3.5 (Brs, 2H), 2.99-2.92 (m, 2H), 2.39 (s, 3H), 2.14-1.95 (m, 4H).

### Example 33

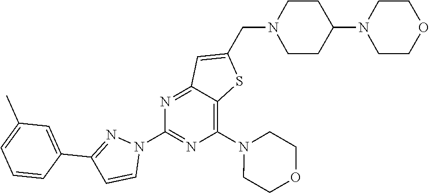 [SMILES: Cc1cccc(-c2ccn(-c3nc(N4CCOCC4)c4sc(CN5CCC(N6CCOCC6)CC5)cc4n3)n2)c1]

### Step 5: 4-(1-((4-morpholino-2-(3-(m-tolyl)-1H-pyrazol-1-yl)thieno[3,2-d]pyrimidin-6-yl)methyl)piperidin-4-yl)morpholine

Following the general procedure of Example 18, 4-(6-(chloromethyl)-2-(3-(m-tolyl)-1H-pyrazol-1-yl)thieno[3,2-d]pyrimidin-4-yl)morpholine (50 mg) is treated with 4-(piperidin-4-yl)morpholine (39.9 mg) to obtain crude (69 mg) as an off white solid. The crude compound is purified by flash RP, eluting with 65-70% acetonitrile/water, which after lyophilization affords the title compound (15 mg; 26%) as an off-white solid. Mass [m/z] 560.3 [M+H]⁺; ¹H-NMR (400 MHz, DMSO-d₆): 8.72 (d, J=2.8 Hz, 1H), 7.78 (Brs, 1H), 7.75 (d, J=8 Hz, 1H), 7.39 (s, 1H), 7.35 (t, J=7.6 Hz 1H), 7.2 (d, J=7.2 Hz, 1H), 7.02 (d, J=2.4 Hz, 1H), 4.01-3.99 (m, 4H), 3.95 (s, 2H), 3.81-3.78 (m, 4H), 3.55-3.61 (m, 4H), 2.99-2.92 (m, 2H), 2.39 (s, 3H), 2.14-1.95 (m, 4H), 1.77-1.75 (m, 1H), 1.47-1.43 (m, 1H).

### Example 34

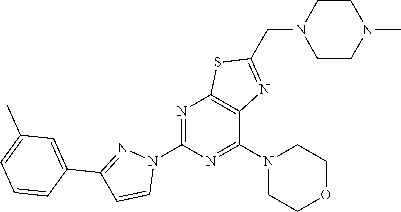 [SMILES: Cc1cccc(-c2ccn(-c3nc(N4CCOCC4)c4nc(CN5CCN(C)CC5)sc4n3)n2)c1]

### Step 6: 4-(2-((4-methylpiperazin-1-yl)methyl)-5-(3-(m-tolyl)-1H-pyrazol-1-yl)thiazolo[5,4-d]pyrimidin-7-yl)morpholine

Following the general procedure of Example 9, 4-(2-(chloromethyl)-5-(3-(m-tolyl)-1H-pyrazol-1-yl)thiazolo[5,4-d]pyrimidin-7-yl)morpholine (40 mg) is reacted with 1-methylpiperazine (19 mg) and the resulting crude is purified by Flash RP chromatography (C18, 6 g column) eluting with 75-80% acetonitrile/water to afford the title compound (13 mg; 28%) as a white solid. Mass [m/z] 491.2 [M+H]⁺; ¹H-NMR (400 MHz, DMSO-d₆): 8.73 (d, J=2.8 Hz, 1H), 7.79 (s, 1H), 7.75 (d, J=7.6 Hz, 1H), 7.35 (t, J=7.6 Hz, 1H), 7.21 (d, J=7.6 Hz, 1H), 7.05 (d, J=2.8 Hz, 1H), 4.35 (br s, 4H), 3.91 (s, 2H), 3.78 (t, J=4.8 Hz, 4H), 2.67 (br s, 4H), 2.39 (s, 3H), 2.22 (s, 4H), 1.23 (s, 3H).

### Example 35

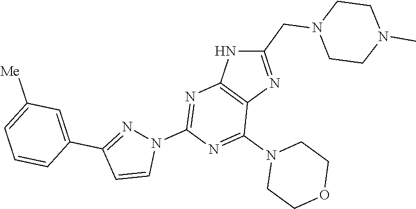 [SMILES: Cc1cccc(-c2ccn(-c3nc(N4CCOCC4)c4nc(CN5CCN(C)CC5)[nH]c4n3)n2)c1]

### Step 6: 4-(8-((4-methylpiperazin-1-yl)methyl)-2-(3-(m-tolyl)-1H-pyrazol-1-yl)-9H-purin-6-yl)morpholine

Following the general procedure of Example 15, 4-(8-(chloromethyl)-9-methyl-2-(3-(m-tolyl)-1H-pyrazol-1-yl)-9H-purin-6-yl)morpholine (30 mg) is reacted with 1-methylpiperazine (27 mg) and the resulting crude product is purified by Prep HPLC (Method-B: Kinetix, EVO, C18, 250×21.2 mm, 5 μm; Mobile phase: [ACN: 0.1% of Formic acid in Water]; time/B %: 0/20, 15/55, 20/70, 21/95), eluting with 60% acetonitrile/water to afford the title compound (4 mg; 20%) as a white solid. Mass [m/z]474.3 [M+H]⁺; ¹H-NMR (400 MHz, CDCl₃): 10.00 (br s, 1H), 8.54 (d, J=2.4 Hz, 1H), 7.86 (s, 1H), 7.72 (d, J=3.2 Hz, 1H), 7.52 (s, 1H), 7.29 (m. 1H), 7.14 (d, J=7.6 Hz, 1H), 6.75 (d, J=2.4 Hz, 1H), 4.38 (br s, 4H), 3.86 (t, J=4.8 Hz, 4H), 3.71 (s, 2H), 2.56 (s, 4H), 2.45 (s, 6H), 2.31 (s, 3H).

### Example 36

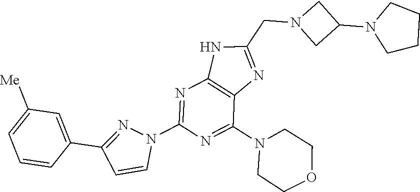 [SMILES: Cc1cccc(-c2ccn(-c3nc(N4CCOCC4)c4nc(CN5CC(N6CCCC6)C5)[nH]c4n3)n2)c1]

### Step 6: 4-(8-((3-(pyrrolidin-1-yl)azetidin-1-yl)methyl)-2-(3-(m-tolyl)-1H-pyrazol-1-yl)-9H-purin-6-yl)morpholine

Following the general procedure of Example 15, 4-(8-(chloromethyl)-9-methyl-2-(3-(m-tolyl)-1H-pyrazol-1-yl)-9H-purin-6-yl)morpholine (30 mg) is reacted with 3-(pyrrolidine-1-yl)azetidine (32 mg) and the resulting crude product is purified by Prep HPLC (Method-B: Kinetix, EVO, C18, 250×21.2 mm, 5 μm; Mobile phase: [ACN: 0.1% of Formic acid in Water]; time/B %: 0/20, 15/55, 20/70, 21/95) eluting with 60% acetonitrile/water to afford the title compound (5 mg; 30%) as a white solid. Mass [m/z]500.3 [M+H]⁺; ¹H-NMR (400 MHz, DMSO-d₆): 8.50 (d, J=2.4 Hz, 1H), 7.85 (s, 1H), 7.72 (d, J=3.6 Hz, 1H), 7.31 (m, 1H), 7.14 (d, J=7.8 Hz, 1H), 6.74 (d, J=2.4 Hz, 1H), 4.34 (s, 4H), 3.86-3.89 (m, 6H), 3.56 (t, J=5.6 Hz, 2H), 3.32-3.37 (m, 2H), 2.66 (Br s, 3H), 2.40 (s, 3H), 1.89 (s, 5H).

### Example 37

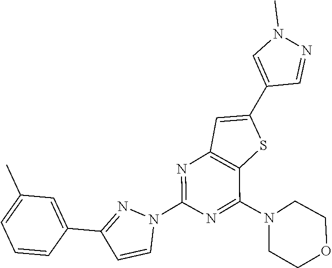 [SMILES: Cc1cccc(-c2ccn(-c3nc(N4CCOCC4)c4sc(-c5cnn(C)c5)cc4n3)n2)c1]

### Step 1: 4-(2-chloro-6-iodothieno[3,2-d]pyrimidin-4-yl)morpholine

To a stirred solution of 4-(2-chlorothieno[3,2-d]pyrimidin-4-yl)morpholine (500 mg, 1.96 mmol, 1 eq.) in THE (8 mL) at −78° C. is added n-BuLi (1.0 M in THF) (2.94 mL, 2.94 mmol, 1.5 eq) and stirred for 15 min, then iodochloroethane (2.94 mL, 2.94 mmol, 1.5 eq) is added at the same temperature. The reaction temperature is allowed to reach room temperature and it is stirred for an additional 4 h. The reaction is monitored by TLC, and after completion of the reaction, it is quenched with saturated NH₄Cl solution (25 mL) and extracted with EtOAc (2×60 mL). The organic layer is dried over Na₂SO₄, filtered, and evaporated under vacuum to obtain the crude as a sticky liquid which is purified by silica gel chromatography (60-120 mesh) eluting with 10-15% EtOAc/hexane, to afford the title compound (300 mg; 40%) as an off white solid. Mass [m/z] 382 [M+H]⁺; ¹H-NMR (400 MHz, DMSO-d₆): 7.25 (s, 1H), 3.90 (t, J=4.4 Hz, 4H), 3.74 (t, J=5.2 Hz, 4H).

### Step 2: 4-(2-chloro-6-(1-methyl-1H-pyrazol-4-yl)thieno[3,2-d]pyrimidin-4-yl) morpholine

To a stirred solution of 4-(2-chloro-6-iodothieno[3,2-d]pyrimidin-4-yl)morpholine (100 mg, 0.26 mmol, 1 eq.), 1-methyl-4-(4,4,5,5-tetramethyl-1,3,2-dioxaborolan-2-yl)-1H-pyrazole (82 mg, 0.39 mmol, 1.5 eq.) in a mixture 1,4-dioxane: water (1:1) (5 mL) is added Pd₂(dba)₃ (12 mg, 0.013 mmol, 0.05 eq.), and tri-tert-butyl-phosphonium tetrafluoroborate (7 mg, 0.023 mmol, 0.09 eq.), followed by K₃PO₄ (111 mg, 0.52 mmol, 2 eq.) and the mixture is degassed for 30 min. The reaction mixture is stirred at 80° C. for 1 h under microwave conditions. TLC monitoring confirms the complete consumption of the starting material. The reaction mixture is filtered through a Celite pad to remove the catalyst impurities and the filtrate is diluted with EtOAc (30 mL) then washed with water (10 mL), dried over Na₂SO₄, filtered, and evaporated under vacuum to produce a sticky liquid. Purification of the crude using silica gel chromatography eluting with 35%-40% EtOAc/hexane affords the title compound (70 mg; 19%) as an off white solid. Mass [m/z]382 [M+H]⁺; ¹H-NMR (400 MHz, DMSO-d₆): 8.75 (s, 1H), 7.79 (br s, 1H), 7.75 (d, J=7.6 Hz, 1H), 7.36 (t, J=11.4 Hz 1H), 7.2 (d, J=7.6 Hz, 1H), 7.05 (d, J=2.8 Hz, 1H), 4.06 (t, J=7 Hz, 4H), 3.829 (t, J=4.8 Hz, 4H), 2.39 (s, 3H) 2.33-2.32 (m, 4H), 1.23 (s, 1H).

### Step 3: 4-(6-(1-methyl-1H-pyrazol-4-yl)-2-(3-(m-tolyl)-1H-pyrazol-1-yl)thieno[3,2-d]pyrimidin-4-yl)morpholine

To a stirred solution of 4-(2-chloro-6-(1-methyl-1H-pyrazol-4-yl)thieno[3,2-d]pyrimidin-4-yl)morpholine (70 mg, 0.20 mmol, 1 eq.), and 3-(m-tolyl)-1H-pyrazole (49 mg, 0.31 mmoL, 1.5 eq.) dissolved in a mixture of toluene: 1,4-dioxane (1:1) (4 mL) is added Pd₂(dba)₃ (19 mg, 0.02 mmol, 0.1 eq.), and t-Bu-XPhos (18 mg, 0.04 mmol, 0.2 eq.), followed by K₃PO₄ (88 mg, 0.41 mmol, 2 eq.), and the mixture is degassed for 30 min.

The reaction mixture is stirred at 120° C. for 6 h. Progress of the reaction is monitored by TLC. After complete consumption of the starting material, the reaction mixture is filtered through a celite pad and the filtrate is diluted with EtOAc (30 mL), washed with water (10 mL), dried over anhydrous Na₂SO₄, filtered, and evaporated under reduced pressure to obtain a brown liquid. The crude product is purified by Flash (C18 reverse phase 12 g column) eluting with 60-70% acetonitrile/water to afford the title compound (20 mg; 21%) as an off white solid. Mass [m/z] 458.1 [M+H]⁺; ¹H-NMR (400 MHz, DMSO-d₆): 8.75 (s, 1H), 7.79 (br s, 1H), 7.75 (d, J=7.6 Hz, 1H), 7.36 (t, J=11.4 Hz 1H), 7.2 (d, J=7.6 Hz, 1H), 7.05 (d, J=2.8 Hz, 1H), 4.06 (t, J=7 Hz 4H), 3.829 (t, J=4.8 Hz, 4H), 2.39 (s, 3H) 2.33-2.32 (m, 4H), 1.23 (s, 1H).

### Example 38

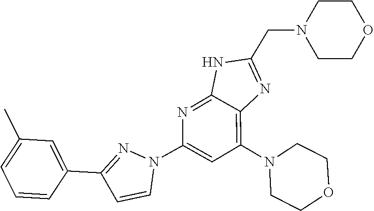 [SMILES: Cc1cccc(-c2ccn(-c3cc(N4CCOCC4)c4nc(CN5CCOCC5)[nH]c4n3)n2)c1]

### Step 9: 4-((7-morpholino-5-(3-(m-tolyl)-1H-pyrazol-1-yl)-3H-imidazo[4,5-b]pyridin-2-yl)methyl)morpholine

Following the general procedure of Example 27, 4-(2-(chloromethyl)-5-(3-(m-tolyl)-1H-pyrazol-1-yl)-3H-imidazo[4,5-b]pyridin-7-yl)morpholine (35 mg) is reacted with morpholine (1.5 eq.) to provide a crude product (42 mg) which is purified by RP flash column chromatography (C18-6 g) eluting with 70% of acetonitrile/water to afford the title compound (15 mg; 38%) as an off white solid. Mass [m/z] 460.4 [M+H]⁺; ¹H-NMR (400 MHz, DMSO-d₆): 9.70 (Brs, 1H), 8.48 (d, J=2.8 Hz, 1H), 7.77 (Brs, 1H), 7.73 (d, J=8 Hz, 1H), 7.34-7.31 (m, 2H), 7.17 (d, J=7.6 Hz 1H), 6.76 (d, J=2.4 Hz, 1H), 4.01-3.99 (m, 4H), 3.95-3.93 (m, 4H), 3.75-3.73 (m, 6H), 2.56 (t, J=4.4 Hz, 4H), 2.43 (s, 3H).

### Example 39

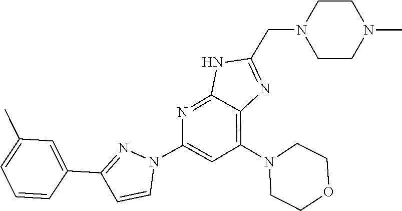 [SMILES: Cc1cccc(-c2ccn(-c3cc(N4CCOCC4)c4nc(CN5CCN(C)CC5)[nH]c4n3)n2)c1]

### Step 9: 4-(2-((4-methylpiperazin-1-yl)methyl)-5-(3-(m-tolyl)-1H-pyrazol-1-yl)-3H-imidazo[4,5-b]pyridin-7-yl)morpholine

Following the general procedure of Example 27, 4-(2-(chloromethyl)-5-(3-(m-tolyl)-1H-pyrazol-1-yl)-3H-imidazo[4,5-b]pyridin-7-yl)morpholine (35 mg) is reacted with 1-methylpiperazine (1.5 eq.) to provide a crude product (42 mg) which is purified by RP flash column chromatography (C18-6 g), eluting with 75% acetonitrile/water to afford the title compound (12 mg; 30%) as an off white solid. Mass [m/z] 473.4 [M+H]⁺; ¹H-NMR (400 MHz, DMSO-d₆): 9.83 (brs, 1H), 8.48 (d, J=2.8 Hz, 1H), 7.77 (brs, 1H), 7.73 (d, J=8 Hz, 1H), 7.34-7.31 (m, 2H), 7.17 (d, J=7.6 Hz 1H), 6.76 (d, J=2.4 Hz, 1H), 4.01-3.99 (m, 4H), 3.95-3.93 (m, 4H), 3.77 (s, 2H), 2.65 (brs, 4H), 2.56 (brs, 3H), 2.43 (s, 3H), 2.37 (s, 3H).

### Example 40

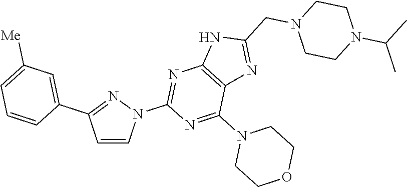 [SMILES: Cc1cccc(-c2ccn(-c3nc(N4CCOCC4)c4nc(CN5CCN(C(C)C)CC5)[nH]c4n3)n2)c1]

### Step 6: 4-(8-((4-isoproylpiperazin-1-yl)methyl)-2-(3-(m-tolyl)-1H-pyrazol-1-yl)-9H-purin-6-yl)morpholine

Following the general procedure of Example 15, 4-(8-(chloromethyl)-9-methyl-2-(3-(m-tolyl)-1H-pyrazol-1-yl)-9H-purin-6-yl)morpholine (40 mg) is reacted with 1-isopropylpiperazine (25 mg) and the resulting crude product is purified by Flash RP chromatography (C18, 6 g column) eluting with 75% acetonitrile/water to afford the title compound (7 mg; 14%) as a white solid. Mass [m/z] 500.2 [M+H]⁺; ¹H-NMR (400 MHz, DMSO-d₆): 8.66 (d, J=4.0 Hz, 1H), 8.32 (s, 1H), 7.79 (s, 1H), 7.71 (d, J=7.6 Hz, 1H), 7.34 (t, J=7.6 Hz, 1H), 7.19 (d, J=7.6 Hz, 1H), 7.0 (d, J=2.4 Hz, 1H), 4.27 (Br s, 4H), 3.77 (t, J=4.8 Hz, 4H), 3.63 (s, 2H), 2.56-2.63 (m, 3H), 2.45 (s, 6H), 2.38 (s, 3H), 0.94 (d, J=6.4 Hz, 6H).

### Example 41

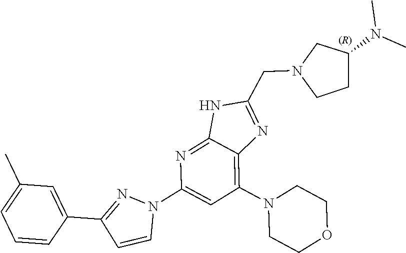

### Step 9: (R)—N,N-dimethyl-1-((7-morpholino-5-(3-(m-tolyl)-1H-pyrazol-1-yl)-3H-imidazo[4,5-b]pyridin-2-yl)methyl)pyrrolidin-3-amine

Following the general procedure of Example 27, 4-(2-(chloromethyl)-5-(3-(m-tolyl)-1H-pyrazol-1-yl)-3H-imidazo[4,5-b]pyridin-7-yl)morpholine (30 mg) is reacted with (R)—N,N-dimethylpyrrolidin-3-amine (1.5 eq.) to provide a crude product (32 mg) which is subjected to Prep HPLC purification (Column: Kinetex EVO, C18, 250×21.2 mm, 5 μm; Mobile phase: [ACN: 0.1% of Formic acid in Water]; time/B %: 0/15, 12/40, 15/50, 17/95), to afford the title compound (3 mg; 8%) as an off-white solid. Mass [m/z] 487.5 [M+H]⁺; ¹H-NMR (400 MHz, DMSO-d₆): 8.62 (brs, 1H), 8.56 (d, J=2.8 Hz, 1H), 7.77 (brs, 1H), 7.73 (d, J=8 Hz, 1H), 7.32 (t, J=7.6, 2H), 7.16 (d, J=7.6 Hz 1H), 6.74 (d, J=2.4 Hz, 1H), 4.01-4.03 (m, 1H), 3.96-3.92 (m, 9H), 3.22-3.09 (m, 3H), 2.76-2.73 (m, 2H), 2.66 (s, 6H), 2.43 (s, 3H), 2.20-2.13 (m, 6H).

### Example 42

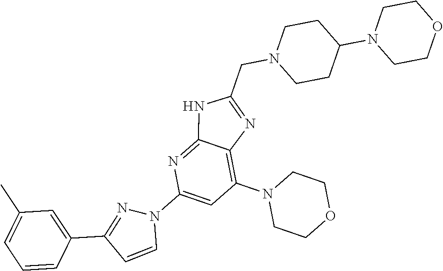 [SMILES: Cc1cccc(-c2ccn(-c3cc(N4CCOCC4)c4nc(CN5CCC(N6CCOCC6)CC5)[nH]c4n3)n2)c1]

### Step 9: 4-(1-((7-morpholino-5-(3-(m-tolyl)-1H-pyrazol-1-yl)-3H-imidazo[4,5-b]pyridin-2-yl)methyl)piperidin-4-yl)morpholine

Following the general procedure of Example 27, 4-(2-(chloromethyl)-5-(3-(m-tolyl)-1H-pyrazol-1-yl)-3H-imidazo[4,5-b]pyridin-7-yl)morpholine (30 mg) is reacted with 4-(piperidin-4-yl)morpholine (1.5 eq.) to provide a crude product (36 mg) which is purified by Prep HPLC (Column: Gemini C18, 250×21.2 mm, 5 μm; Mobile phase: [ACN: 0.1% of Formic acid in Water]; time/B %: 0/40, 14/55, 25/90, 26/95), to afford the title compound (5 mg; 13%) as off-white solid. Mass [m/z] 543.4 [M+H]⁺; ¹H-NMR (400 MHz, DMSO-d₆): 8.65 (brs, 1H), 8.56 (d, J=2.8 Hz, 1H), 7.77 (brs, 1H), 7.73 (d, J=8 Hz, 1H), 7.34 (t, J=7.6, 2H), 7.16 (d, J=7.6 Hz 1H), 6.74 (d, J=2.4 Hz, 1H), 4.06 (m, 1H), 3.95-3.91 (m, 7H), 3.22-3.08 (m, 3H), 2.75-2.67 (m, 2H), 2.65 (s, 6H), 2.43 (s, 3H), 2.27-2.12 (m, 2H).

### Example 43

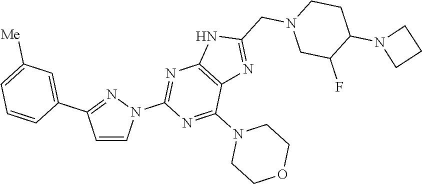 [SMILES: Cc1cccc(-c2ccn(-c3nc(N4CCOCC4)c4nc(CN5CCC(N6CCC6)C(F)C5)[nH]c4n3)n2)c1]

### Step 6: 4-(8-((4-(azetidin-1-yl)-3-fluoropiperidin-1-yl)methyl-2-(3-m-tolyl)-1H-pyrazol-1-yl)-9H-purin-6-yl)morpholine

Following the general procedure of Example 15, 4-(8-(chloromethyl)-9-methyl-2-(3-(m-tolyl)-1H-pyrazol-1-yl)-9H-purin-6-yl)morpholine (40 mg) is reacted with 4-(azetidin-1-yl)-3-fluoropiperidine (30.9 mg) and the resulting crude is purified by Prep HPLC (Method-B: Kinetix, EVO, C18, 250×21.2 mm, 5 μm; Mobile phase: [ACN: 0.1% of Formic acid in Water]; time/B %: 0/20, 15/55, 20/70, 21/95) eluting with 55% acetonitrile/water to afford the title compound (2 mg; 4%) as a white solid. Mass [m/z]532.4 [M+H]⁺; ¹H-NMR (400 MHz, DMSO-d₆): 13.18 (br s, 1H), 8.66 (d, J=2.8 Hz, 1H), 8.36 (s, 1H), 7.76 (s, 1H), 7.71 (d, J=8.0 Hz, 1H), 7.34 (t, J=7.8 Hz, 1H), 7.17 (d, J=7.8 Hz, 1H), 6.98 (d, J=2.8 Hz), 4.54-4.67 (m, 1H), 4.27 (br s, 4H), 3.76-3.78 (t, J=4.8 Hz, 4H), 3.70 (s, 2H), 3.10-3.16 (t, J=9.8 Hz, 4H), 2.92 (m, 1H), 2.65 (m, 1H). 2.41 (s, 3H), 2.22 (m, 1H), 2.06 (m, 2H), 1.5 (m, 2H).

### Example 44

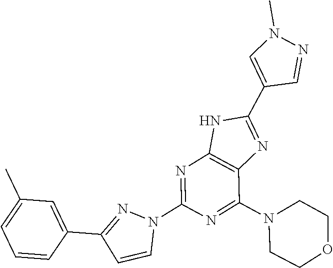 [SMILES: Cc1cccc(-c2ccn(-c3nc(N4CCOCC4)c4nc(-c5cnn(C)c5)[nH]c4n3)n2)c1]

### Step 1: 4-(2-chloro-8-(1-methyl-1H-pyrazol-4-yl)-9-(tetrahydro-2H-pyran-2-yl)-9H-purin-6-yl)morpholine

To a stirred solution of 4-(2-chloro-8-iodo-9-(tetrahydro-2H-pyran-2-yl)-9H-purin-6-yl)morpholine (200 mg, 0.445 mmol, 1 eq.) and 1-methyl-4-(4,4,5,5-tetramethyl-1,3,2-dioxaborolan-2-yl)-1H-pyrazole (80 mg, 0.40 mmol, 0.9 eq) dissolved in a mixture of 1,4-dioxane: water (4:1) is added Pd(dppf)Cl₂-DCM (32 mg, 0.044 mmol, 0.1 eq.), followed by K₂CO₃ (112 mg, 0.89 mmol, 2 eq.) and the mixture is degassed for 30 min.

The reaction mixture is stirred at 80° C. for 3 h. Progress of the reaction is monitored by TLC and after complete consumption of the starting material, it is filtered through a Celite pad and the resulting filtrate is diluted with ethyl acetate (20 mL) and washed with water (10 mL). Finally, the organic layer is dried over anhydrous Na₂SO₄, filtered, and evaporated under reduced pressure to obtain a brown liquid which is purified by silica gel chromatography eluting with 35%-40% EtOAc/petroleum ether to afford the title compound (100 mg; 55%) as an off-white solid. Mass [m/z] 404.1 [M+H]⁺; ¹H-NMR (400 MHz, DMSO-d₆): 8.26 (s, 1H), 7.92 (d, J=8.0 Hz, 1H), 5.65-5.62 (m, 1H), 4.16-4.13 (m, 2H), 4.02 (d, J=7.2 Hz, 2H), 3.95 (s, 3H), 3.76-3.66 (m, 5H), 2.59-2.55 (m, 1H), 1.93-190 (m, 1H), 1.70-1.64 (m, 3H), 1.61-1.56 (m, 2H).

### Step 2: 4-(8-(1-methyl-1H-pyrazol-4-yl)-9-(tetrahydro-2H-pyran-2-yl)-2-(3-(m-tolyl)-1H-pyrazol-1-yl)-9H-purin-6-yl)morpholine

To a stirred solution of 4-(2-chloro-8-(1-methyl-1H-pyrazol-4-yl)-9-(tetrahydro-2H-pyran-2-yl)-9H-purin-6-yl)morpholine (100 mg, 0.24 mmol, 1 eq.), and 3-(m-tolyl)-1H-pyrazole (58 mg, 0.37 mmol, 1.5 eq.) in a mixture of toluene: 1,4-dioxane (1:1) (5 mL) is added Pd₂(dba)₃ (22 mg, 0.02 mmol, 0.1 eq.), and t-Bu-XPhos (21 mg, 0.04 mmol, 0.2 eq.), followed by K₃PO₄ (105 mg, 0.49 mmol, 2 eq.), and the mixture is degassed for 30 min. The reaction mixture is stirred at 120° C. for 5 h and progress is monitored by TLC.

After complete consumption of the starting material, the mixture is filtered through a Celite bed. The filtrate is diluted with EtOAc (20 mL), washed with water (10 mL), dried over anhydrous Na₂SO₄, filtered, and evaporated under reduced pressure to obtain a brown liquid. The crude compound is purified using silica gel chromatography (60-120 mesh) eluting with 50-55% ethyl acetate/petroleum ether to afford the title compound (60 mg; 46%) as an off white solid. Mass [m/z] 526.2 [M+H]⁺; ¹H-NMR (400 MHz, DMSO-d₆): 8.71 (d, J=2.4 Hz, 1H), 8.28 (s, 1H), 7.96 (s, 1H), 7.82-7.76 (m, 3H), 7.47-7.46 (m, 1H), 7.36 (t, J=7.6 Hz, 1H), 7.20 (d, J=7.6 Hz, 1H), 7.02 (t, J=2.4 Hz, 1H), 5.78 (d, J=7.6 Hz, 1H), 4.33 (br s, 3H), 4.20 (d, J=9.6 Hz, 1H), 3.96 (s, 3H), 3.78 (t, J=4.4 Hz, 4H), 2.76-7.73 (m, 1H), 2.49 (s, 3H), 1.98 (s, 1H), 1.76-1.73 (m, 3H), 1.61 (s, 1H).

### Step 3: 4-(8-(1-methyl-1H-pyrazol-4-yl)-2-(3-(m-tolyl)-1H-pyrazol-1-yl)-9H-purin-6-yl)morpholine

To a stirred solution of 4-(8-(1-methyl-1H-pyrazol-4-yl)-9-(tetrahydro-2H-pyran-2-yl)-2-(3-(m-tolyl)-1H-pyrazol-1-yl)-9H-purin-6-yl)morpholine (60 mg, 0.11 mmol, 1 eq.) in 1,4-dioxane (2 mL) at 0° C. is added 4 M HCl in 1,4-dioxane (3 mL). The reaction mixture is brought to room temperature and stirred for another 4 h. The progress of the reaction is monitored by TLC, and after completion of the reaction, it is evaporated under vacuum to obtain a sticky liquid which is purified by Flash chromatography (C18 reverse phase 12 g column) eluting with 65-70% acetonitrile/water to afford the title compound (10 mg; 20%) as an off-white solid. Mass [m/z] 442.2 [M+H]⁺; ¹H-NMR (400 MHz, DMSO-d₆): 13.55 (br s, 1H), 8.67 (d, J=2.8 Hz, 1H), 8.29 (s, 1H), 7.99 (s, 1H), 7.77 (s, 1H), 7.73 (d, J=7.6 Hz, 1H), 7.34 (t, J=7.6 Hz, 1H), 7.18 (d, J=7.6 Hz, 1H), 7.0 (d, J=2.4 Hz, 1H), 4.31 (br s, 4H), 3.92 (s, 3H), 3.79 (t, J=4.8 Hz, 4H), 2.44 (s, 3H).

### Example 45

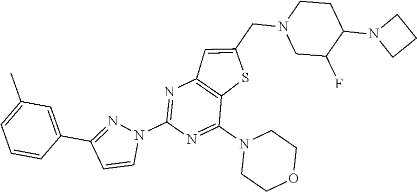 [SMILES: Cc1cccc(-c2ccn(-c3nc(N4CCOCC4)c4sc(CN5CCC(N6CCC6)C(F)C5)cc4n3)n2)c1]

### Step 5: 4-(1-((4-morpholino-2-(3-(m-tolyl)-1H-pyrazol-1-yl)thieno[3,2-d]pyrimidin-6-yl)methyl)piperidin-4-yl)morpholine

Following the general procedure of Example 18, 4-(6-(chloromethyl)-2-(3-(m-tolyl)-1H-pyrazol-1-yl)thieno[3,2-d]pyrimidin-4-yl)morpholine (40 mg, 1 eq.) in IPA (4 mL) is treated with 4-(azetidin-1-yl)-3-fluoropiperidine (29.5 mg, 2 eq.) and DIPEA (36 mg, 0.28 mmol, 3 eq) at room temperature. The reaction mixture is refluxed for 6-8 h, progress of the reaction is monitored by TLC. After completion of the reaction, it is diluted with ethyl acetate (2×10 mL) and washed with water (10 mL). Finally, the organic layers are dried over anhydrous Na₂SO₄, filtered, and evaporated under reduced pressure to obtain a crude product (55 mg) as an off-white solid. The crude compound is purified by Prep HPLC (Method-B: Kinetex, EVO, C18, 250×21.2 mm, 5 μm; Mobile phase: [ACN: 0.1% of Formic acid in Water]; time/B %: 0/15, 12/40, 15/50, 17/95), which after lyophilization affords the title compound (8 mg; 16%) as an off-white solid. Mass [m/z]548.4 [M+H]⁺; ¹H-NMR (400 MHz, DMSO-d₆): 8.72 (d, J=2.8 Hz, 1H), 7.79 (Brs, 1H), 7.75 (d, J=8 Hz, 1H), 7.40 (s, 1H), 7.35 (t, J=7.6 Hz 1H), 7.2 (d, J=7.2 Hz, 1H), 7.02 (d, J=2.4 Hz, 1H), 4.01-3.99 (m, 4H), 3.86 (s, 2H), 3.81-3.79 (m, 4H), 2.39 (s, 3H), 2.19 (Brs, 3H), 1.23 (s, 2H).

### Example 46

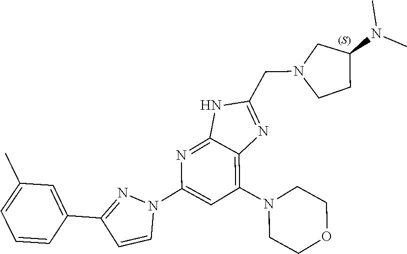

### Step 9: (S)—N,N-dimethyl-1-((7-morpholino-5-(3-(m-tolyl)-1H-pyrazol-1-yl)-3H-imidazo[4,5-b]pyridin-2-yl)methyl)pyrrolidin-3-amine

Following the general procedure of Example 27, 4-(2-(chloromethyl)-5-(3-(m-tolyl)-1H-pyrazol-1-yl)-3H-imidazo[4,5-b]pyridin-7-yl)morpholine (30 mg) is reacted with (S)—N,N-dimethylpyrrolidin-3-amine (1.5 eq.) to provide a crude product (34 mg) which is subjected to Prep HPLC purification (Column: Kinetex, EVO, C18, 250×21.2 mm, 5 μm; Mobile phase: [ACN: 0.1% of Formic acid in Water]; time/B %: 0/15, 12/40, 15/50, 17/95), to afford the title compound (2.1 mg; 6%) as an off-white solid. Mass [m/z]543.4 [M+H]⁺; ¹H-NMR (400 MHz, DMSO-d₆):): 8.65 (brs, 1H), 8.56 (d, J=2.8 Hz, 1H), 7.77 (brs, 1H), 7.73 (d, J=8 Hz, 1H), 7.34 (t, J=7.6, 2H), 7.16 (d, J=7.6 Hz 1H), 6.74 (d, J=2.4 Hz, 1H), 4.06 (m, 1H), 3.95-3.91 (m, 7H), 3.22-3.08 (m, 3H), 2.75-2.67 (m, 2H), 2.65 (s, 6H), 2.43 (s, 3H), 2.27-2.12 (m, 2H).

### Example 47

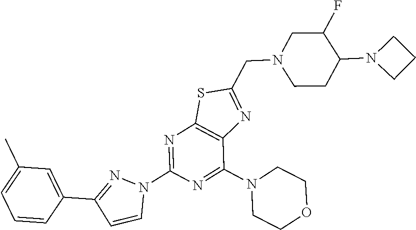 [SMILES: Cc1cccc(-c2ccn(-c3nc(N4CCOCC4)c4nc(CN5CCC(N6CCC6)C(F)C5)sc4n3)n2)c1]

### Step 6: 4-(2-((4-(azetidin-1-yl)-3-fluoropiperidin-1-yl)methyl)-5-(3-(m-tolyl)-1H-pyrazol-1-yl)thiazolo[5,4-d]pyrimidin-7-yl)morpholine

Following the general procedure of Example 9, 4-(2-(chloromethyl)-5-(3-(m-tolyl)-1H-pyrazol-1-yl)thiazolo[5,4-d]pyrimidin-7-yl)morpholine (40 mg) is reacted with 4-(azetidin-1-yl)-3-fluoropiperidine (30 mg) and the resulting crude is purified by Prep HPLC (Method-B: X-Bridge-C18, 250×19.1 mm, 5 μm; Mobile phase: [ACN: 0.1% of Formic acid in Water]; time/B %: 0/20, 15/50, 25/60, 26/95) to afford the title compound (11 mg; 21%) as an off white solid. Mass [m/z] 549.3 [M+H]⁺; ¹H-NMR (400 MHz, DMSO-d₆): 8.75 (d, J=2.8 Hz, 1H), 7.79 (s, 1H), 7.75 (d, J=7.6 Hz, 1H), 7.36 (t, J=7.6 Hz, 1H), 7.22 (d, J=7.6 Hz, 1H), 7.07 (d, J=2.8 Hz, 1H), 5.12 (d, J=50.41H), 4.34-4.20 (m, 6H), 4.04 (s, 4H), 3.8-3.78 (m, 6H), 3.41-3.35 (m, 1H), 3.09 (d, J=11.6, 1H), 2.39 (s, 3H), 2.33-2.08 (m, 2H), 1.9-1.88 (m, 1H), 1.68-1.64 (m, 1H), 1.23 (s, 1H).

### Example 48

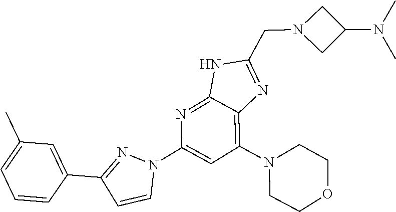 [SMILES: Cc1cccc(-c2ccn(-c3cc(N4CCOCC4)c4nc(CN5CC(N(C)C)C5)[nH]c4n3)n2)c1]

### Step 9: N,N-dimethyl-1-((7-morpholino-5-(3-(m-tolyl)-1H-pyrazol-1-yl)-3H-imidazo[4,5-b]pyridin-2-yl)methyl)azetidin-3-amine

Following the general procedure of Example 27, 4-(2-(chloromethyl)-5-(3-(m-tolyl)-1H-pyrazol-1-yl)-3H-imidazo[4,5-b]pyridin-7-yl)morpholine (30 mg) is reacted with N,N-dimethylazetidin-3-amine (1.5 eq.) to provide a crude product (34 mg) which is purified by Prep HPLC (Column: Gemini C18, 250×21.2 mm, 5 μm; Mobile phase: [ACN: 0.1% of Formic acid in Water]; time/B %: 0/40, 14/55, 20/80, 26/95) to afford the title compound (2 mg; 6%) as off-white solid. Mass [m/z] 543.4 [M+H]⁺; ¹H-NMR (400 MHz, DMSO-d₆): 8.65 (Brs, 1H), 8.56 (d, J=2.8 Hz, 1H), 7.77 (Brs, 1H), 7.73 (d, J=8 Hz, 1H), 7.34 (t, J=7.6, 2H), 7.16 (d, J=7.6 Hz 1H), 6.74 (d, J=2.4 Hz, 1H), 4.06 (m, 1H), 3.95-3.91 (m, 7H), 3.22-3.08 (m, 3H), 2.75-2.67 (m, 2H), 2.65 (s, 6H), 2.43 (s, 3H), 2.27-2.12 (m, 2H).

### Example 49

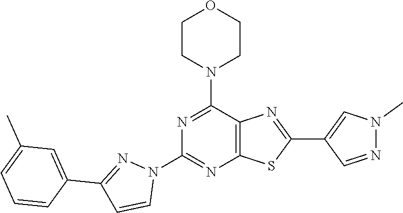 [SMILES: Cc1cccc(-c2ccn(-c3nc(N4CCOCC4)c4nc(-c5cnn(C)c5)sc4n3)n2)c1]

### Step 1: 4-(5-chloro-2-iodothiazolo[5,4-d]pyrimidin-7-yl)morpholine

To a stirred solution of 4-(5-chlorothiazolo[5,4-d]pyrimidin-7-yl)morpholine (100 mg, 0.39 mmol, 1 eq.) in THE (8 mL) at −78° C. is added n-BuLi (1.0 M in THF) (0.7 mL, 0.78 mmol, 2 eq) followed by iodochloroethane (0.2 mL, 0.39 mmol, 1 eq). Then the reaction is warmed to room temperature and the reaction is stirred for 4 h. The progress of the reaction is monitored by TLC and after completion of the reaction, it is quenched with saturated NH₄Cl solution (25 mL) and extracted with EtOAc (2×50 mL). The organic layer is dried over Na₂SO₄, filtered, and evaporated under vacuum to obtain a sticky liquid which is purified by silica gel column chromatography (60-120 mesh) eluting with 5-10% EtOAc/hexane to afford the title compound (75 mg; 51%) as a pale-yellow solid. Mass [m/z] 383 [M+H]⁺; ¹H-NMR (400 MHz, CDCl₃): 4.36 (br s, 4H), 3.85-3.82 (m, 4H).

### Step 2: 4-(5-chloro-2-(1-methyl-1H-pyrazol-4-yl)thiazolo[5,4-d]pyrimidin-7-yl)morpholine

To a stirred solution of 4-(5-chloro-2-iodothiazolo[5,4-d]pyrimidin-7-yl)morpholine (75 mg, 0.19 mmol, 1 eq.), and 1-methyl-4-(4,4,5,5-tetramethyl-1,3,2-dioxaborolan-2-yl)-1H-pyrazole (36 mg, 0.19 mmol, 1 eq.) in a mixture of 1,4-dioxane: water (4:1) (5 mL) is added Pd(dppf)Cl₂-DCM (15 mg, 0.019 mmol, 0.1 eq.), followed by K₂CO₃ (34 mg, 0.38 mmol, 2 eq.), and the mixture is degassed for 20 min. The reaction mixture is stirred at 80° C. for 16 h. Progress of the reaction is monitored by TLC. After complete consumption of the starting material, the mixture is filtered through a Celite pad. The filtrate is diluted with ethyl acetate (20 mL), and washed with water (10 mL). Finally, the organic layer is dried over Na₂SO₄, filtered, and evaporated under vacuum to obtain a sticky liquid which after purification by silica gel chromatography (60-120 mesh) eluting with 35-40% EtOAc/hexane affords the title compound (50 mg; 76%) as an off white solid. Mass [m/z]337.1 [M+H]⁺; ¹H-NMR (400 MHz, CDCl₃): 7.93 (d, J=4.0 Hz, 1H), 7.89 (s, 1H), 4.41-4.38 (m, 4H), 3.98 (s, 3H), 3.85 (t, J=4.8 Hz 4H).

### Step 3:4-(2-(1-methyl-1H-pyrazol-4-yl)-5-(3-(m-tolyl)-1H-pyrazol-1-yl)thiazolo[5,4-d]pyrimidin-7-yl)morpholine

To a stirred solution of 4-(5-chloro-2-(1-methyl-1H-pyrazol-4-yl)thiazolo[5,4-d]pyrimidin-7-yl)morpholine (50 mg, 0.14 mmol, 1 eq.), and 3-(m-tolyl)-1H-pyrazole (20 mg, 0.14 mmol, 1 eq.) in a mixture of toluene: 1,4-dioxane (1:1) (6 mL) is added Pd₂(dba)₃ (27 mg, 0.02 mmol, 0.2 eq.), and t-Bu-XPhos (25 mg, 0.05 mmol, 0.2 eq.), followed by K₃PO₄ (63 mg, 0.29 mmol, 2 eq.), and the mixture is degassed for 20 min. The reaction mixture is stirred at 100° C. for 6 h, cooled to room temperature and passed through a Celite pad. The filtrate is diluted with EtOAc (20 mL) and washed with water (10 mL). Finally, the organic layer is dried over anhydrous Na₂SO₄, filtered, and evaporated under reduced pressure to obtain a brown liquid which is purified by Flash chromatography (C18 reverse phase 12 g column) eluting with 65-70% acetonitrile/water to afford the title compound (22 mg; 32%) as an off-white solid. Mass [m/z] 459.1 [M+H]⁺; ¹H-NMR (400 MHz, CDCl₃): 8.55 (d, J=2.8 Hz, 1H), 7.95 (d, J=4.0 Hz, 1H), 7.90 (s, 1H), 7.87 (s, 1H), 7.74 (d, J=7.6 Hz, 1H), 7.31 (t, J=7.6 Hz, 1H), 7.17 (d, J=7.6 Hz, 1H), 6.78 (d, J=2.8 Hz, 1H), 4.47 (br s, 4H), 3.99 (s, 3H), 3.91 (t, J=4.8 Hz, 4H), 2.41 (s, 3H).

### Example 50

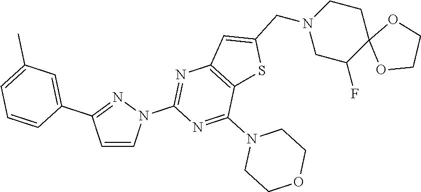 [SMILES: Cc1cccc(-c2ccn(-c3nc(N4CCOCC4)c4sc(CN5CCC6(OCCO6)C(F)C5)cc4n3)n2)c1]

### Step 5: 6-fluoro-8-((4-morpholino-2-(3-(m-tolyl)-1H-pyrazol-1-yl)thieno[3,2-d]pyrimidin-6-yl)methyl)-1,4-dioxa-8-azaspiro[4.5]decane

Following the general procedure of Example 45, 4-(6-(chloromethyl)-2-(3-(m-tolyl)-1H-pyrazol-1-yl)thieno[3,2-d]pyrimidin-4-yl)morpholine (50 mg) is treated with 6-fluoro-1,4-dioxa-8-azaspiro[4.5]decane (37.4 mg) to obtain crude (62 mg) as an off-white solid. The crude compound is purified by flash RP column chromatography (C18, 6 g column), product is eluting with 65-70% acetonitrile/water and after lyophilization affords the title compound (15 mg; 26%) as an off-white solid. Mass [m/z] 551.3 [M+H]⁺; ¹H-NMR (400 MHz, DMSO-d₆): 8.73 (s, 1H), 7.79 (s, 1H), 7.73 (d, J=8 Hz, 1H), 7.42 (s, 1H), 7.35 (t, J=7.6 Hz 1H), 7.20 (d, J=2.4 Hz, 1H), 4.01-3.80 (m, 10H), 3.93-3.78 (m, 4H), 3.75-3.73 (m, 6H), 2.85 (br m, 2H), 2.43 (s, 3H), 1.84 (br m, 1H), 1.65 (br m, 1H).

### Example 51

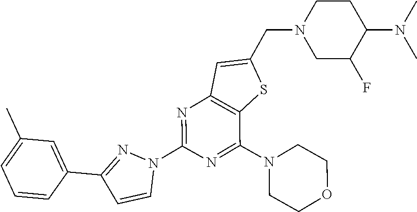 [SMILES: Cc1cccc(-c2ccn(-c3nc(N4CCOCC4)c4sc(CN5CCC(N(C)C)C(F)C5)cc4n3)n2)c1]

### Step 5: 3-fluoro-N,N-dimethyl-1-((4-morpholino-2-(3-(m-tolyl)-1H-pyrazol-1-yl)thieno[3,2-d]pyrimidin-6-yl)methyl)piperidin-4-amine

Following the general procedure of Example 45, 4-(6-(chloromethyl)-2-(3-(m-tolyl)-1H-pyrazol-1-yl)thieno[3,2-d]pyrimidin-4-yl)morpholine (25 mg) is treated with 3-fluoro-N,N-dimethylpiperidin-4-amine (17.5 mg) to obtain crude (40 mg) as an off-white solid.

The crude compound is purified by Prep HPLC (Method-B: Kinetex, EVO, C18, 250×21.2 mm, 5 μm; Mobile phase: [ACN: 0.1% of Formic acid in Water]; time/B %: 0/10, 12/35, 15/60, 20/95), and after lyophilization affords the title compound (2.4 mg) as off-white solid. Mass [m/z] 536.3 [M+H]⁺; ¹H-NMR (400 MHz, DMSO-d₆): 8.73 (d, J=2.8 Hz, 1H), 7.79 (br s, 1H), 7.75 (d, J=8 Hz, 1H), 7.41 (s, 1H), 7.35 (t, J=7.6 Hz 1H), 7.2 (d, J=7.2 Hz, 1H), 7.03 (d, J=2.4 Hz, 1H), 4.98-4.86 (m, 1H), 4.01-3.99 (m, 4H), 3.95 (s, 2H), 3.81-3.78 (m, 4H), 3.13-3.33 (m, 1H), 2.99-2.96 (m, 1H), 2.39 (s, 3H), 2.24 (s, 6H), 2.14-2.22 (m, 2H), 1.8-1.77 (m, 1H), 1.6-1.69 (m, 1H).

### Example 52

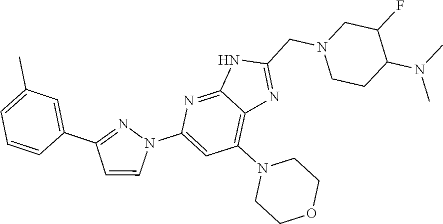 [SMILES: Cc1cccc(-c2ccn(-c3cc(N4CCOCC4)c4nc(CN5CCC(N(C)C)C(F)C5)[nH]c4n3)n2)c1]

### Step 9: 3-fluoro-N,N-dimethyl-1-((7-morpholino-5-(3-(m-tolyl)-1H-pyrazol-1-yl)-3H-imidazo[4,5-b]pyridin-2-yl)methyl)piperidin-4-amine

Following the procedure of Example 27 for steps 1 to 8, to a stirred solution of 4-(2-(chloromethyl)-5-(3-(m-tolyl)-1H-pyrazol-1-yl)-3H-imidazo[4,5-b]pyridin-7-yl)morpholine (50 mg, 0.12 mmol, 1 eq.) in IPA (5 mL) is added 3-fluoro-N,N-dimethylpiperidin-4-amine (27 mg, 0.18 mmol, 1.5 eq) and DIPEA (47 mg, 0.36 mmol, 3 eq) at room temperature. The reaction mixture is heated at reflux for 16 h and progress of the reaction is monitored by TLC. After completion of the reaction, it is diluted with EtOAc (30 mL) and washed with water (10 mL). Finally, the organic layer is dried over anhydrous Na₂SO₄ and evaporated under reduced pressure to obtain crude as an off white solid. The crude product is purified by Prep HPLC (Method-B: Kinetex, EVO, C18, 250×21.2 mm, 5 μm; Mobile phase: [ACN: 0.1% of Formic acid in Water]; time/B %: 0/15, 12/40, 15/50, 17/95), and after lyophilization affords the title compound (9.5 mg; 15%) as an off-white solid. Mass [m/z] 519.3 [M+H]⁺; ¹H-NMR (400 MHz, DMSO-d₆): 12.89 (Brs, 1H), 8.52 (d, J=2.8 Hz, 1H), 7.78 (brs, 1H), 7.75 (brs, 1H), 7.35 (t, J=7.6, 2H), 7.19 (d, J=7.6 Hz 1H), 7.01 (d, J=2.4 Hz, 1H), 4.96 (d, J=49.6 Hz, 1H), 3.94 (Brs, 4H), 3.83-3.82 (m, 4H), 3.67 (s, 2H), 3.08 (t, J=12.4, 1H), 2.94 (d, J=11.2, 1H), 2.34 (s, 3H), 2.22 (s, 6H), 2.19-2.05 (m, 2H), 1.81-1.72 (m, 2H).

### Example 53

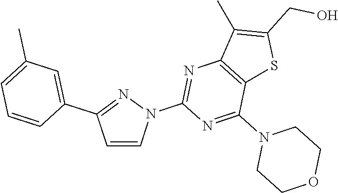 [SMILES: Cc1cccc(-c2ccn(-c3nc(N4CCOCC4)c4sc(CO)c(C)c4n3)n2)c1]

### Step 1: 7-bromothieno[3,2-d]pyrimidine-2,4 (1H,3H)-dione

To a solution of thieno[3,2-d]pyrimidine-2,4 (1H,3H)-dione (12 g, 71.42 mmol, 1 eq) in AcOH (150 mL) at 25° C. is added bromine (11 mL). The temperature of the reaction is raised to 80° C. and stirred for 16 h. The progress of the reaction is monitored by TLC. After completion of the reaction, it is cooled to room temperature and poured into ice cold water (200 mL) and stirred for 15 min, precipitate solid is filtered and first washed with saturated sodium thiosulphate (100 mL) then with saturated sodium bicarbonate (100 mL) and water (100 mL). The obtained solid is vacuum filtered and co-distilled with toluene to obtain the title compound (15 g; 88%) as an off-white solid. Mass [m/z] 247.0 [M+H]⁺; ¹H-NMR (400 MHz, CDCl₃): 7.41 (d, J=1.2 Hz, 1H), 4.0 (t, J=4.8 Hz, 4H), 3.86 (t, J=4.8 Hz, 4H), 2.42 (s, 3H).

### Step 2: 7-bromo-2,4-dichlorothieno[3,2-d]pyrimidine

A solution of 7-bromothieno[3,2-d]pyrimidine-2,4 (1H,3H)-dione (5 g, 20.32 mmol, 1 eq) in POCl₃ (50 mL) is heated to 100° C. and stirred for 16 h. The progress of the reaction is monitored by TLC. After the reaction is completed, it is cooled to room temperature and evaporated to obtain a crude solid (15 g). Ice cold water is added to the brown gummy residue and it is stirred for 15 min. A solid precipitates, which is filtered and dried under vacuum to afford the title compound (4.1 g; 71%) as a pale brown solid. Mass [m/z] 284.9 [M+H]⁺; ¹H-NMR (400 MHz, CDCl₃): 8.11 (s, 1H).

### Step 3: 4-(7-bromo-2-chlorothieno[3,2-d]pyrimidin-4-yl)morpholine

To a solution of 7-bromo-2,4-dichlorothieno[3,2-d]pyrimidine (2 g, 7.09 mmol, 1 eq) in MeOH (20 mL) is added morpholine (1 mL, 7.09 mmol, 1 eq) at room temperature and the reaction is stirred for an additional 3 h. The progress of the reaction is monitored by TLC. After completion, the reaction mixture is filtered and washed with MeOH (10 mL). The resulting solid is dried under vacuum to afford the title compound (1.5 g; 65%) as a pale brown solid. Mass [m/z] 336.0 [M+H]⁺; ¹H-NMR (400 MHz, CDCl₃): 7.79 (s, 1H), 4.01 (t, J=4.4 Hz, 4H), 3.86 (t, J=2.0 Hz, 4H).

### Step 4: 4-(2-chloro-7-methylthieno[3,2-d]pyrimidin-4-yl)morpholine

To a degassed solution of 4-(7-bromo-2-chlorothieno[3,2-d]pyrimidin-4-yl)morpholine (6.5 g, 19.51 mmol, 1 eq) in 1,4-dioxane: water (8:2) (20 mL), is added methylboronic acid (2.3 g, 39.03 mmol, 2 eq), and K₂CO₃ (5.3 g, 39.03 mmol, 3 eq), followed by Pd(dppf)Cl₂ (2.8 g, 3.90 mmol, 0.2 eq) in 1,4-dioxane: water (1:4; 100 mL). The reaction temperature is raised to 100° C. and the reaction is stirred for 16 h. The progress of the reaction is monitored by TLC. After completion of the reaction, it is cooled to room temperature. The reaction is filtered through a Celite pad and washed with ethyl acetate. The filtrate is evaporated under reduced pressure to obtain a crude product (5 g) as a brown gummy liquid. The crude product is purified by chromatography on a silica gel column (60-120 mesh) eluting with 15-20% EtOAc in hexane. Selected product fractions are combined and concentrated to afford the title compound (2.6 g; 49%) as a white solid. Mass [m/z] 270.0 [M+H]⁺; ¹H-NMR (400 MHz, CDCl₃): 7.41 (d, J=1.2 Hz, 1H), 4.0 (t, J=4.8 Hz, 4H), 3.86 (t, J=4.8 Hz, 4H), 2.42 (s, 3H).

### Step 5: 2-chloro-7-methyl-4-morpholinothieno[3,2-d]pyrimidine-6-carbaldehyde

To a solution of 4-(2-chloro-7-methylthieno[3,2-d]pyrimidin-4-yl)morpholine (1.5 g, 5.57 mmol, 1 eq) in dry THE (30 ml), is added n-BuLi (2.5 M in hexane) (5 mL, 11.15 mmol, 2 eq) at −78° C. and the solution is stirred for 1 h, then DMF (3 mL, 33.4 mmol, 6 eq) is added at −78° C. The reaction mixture is stirred at the same temperature for another 2 h. The progress of the reaction is monitored by TLC. After completion of the reaction, it is quenched with saturated ammonium chloride solution (50 mL) and extracted with ethyl acetate (2×50 mL). The organic layer is dried over Na₂SO₄, filtered, and evaporated under reduced pressure to provide a pale-yellow solid (1.8 g) which is triturated with n-pentane to afford the title compound (1.2 g; 75%) as a yellow solid. Mass [m/z] 298.1 [M+H]⁺; ¹H-NMR (400 MHz, CDCl₃): 10.38 (s, 1H), 4.4 (t, J=4.8 Hz, 4H), 3.84 (t, J=2.4 Hz, 4H), 2.76 (s, 3H).

### Step 6: (2-chloro-7-methyl-4-morpholinothieno[3,2-d]pyrimidin-6-yl)methanol:#

To a solution of 2-chloro-7-methyl-4-morpholinothieno[3,2-d]pyrimidine-6-carbaldehyde (1.5 g, 5.38 mmol, 1 eq) in dry THE and MeOH (30 ml), is added NaBH₄ (0.4 mg, 10.77 mmol, 2 eq) at 0° C. The reaction mixture is stirred at the same temperature for 2 h. The progress of the reaction is monitored by TLC. After completion, the reaction mixture is quenched with water (20 mL) and extracted with EtOAc (2×50 mL). The combined organic layer is dried over Na₂SO₄, filtered, and evaporated under reduced pressure to provide a yellow solid (1.5 g), which is triturated with n-pentane to afford the title compound (1.3 g; 81%) as a yellow solid. Mass [m/z] 300.1 [M+H]⁺; ¹H-NMR (400 MHz, DMSO-d₆): 5.91 (s, 1H), 4.80 (d, J=5.2 Hz, 2H), 4.4 (t, J=4.4 Hz, 4H), 3.74 (t, J=4.8 Hz, 4H), 2.19 (s, 3H).

### Step 7: (7-methyl-4-morpholino-2-(3-(m-tolyl)-1H-pyrazol-1-yl)thieno[3,2-d]pyrimidin-6-yl)methanol

To a solution of compound (2-chloro-7-methyl-4-morpholinothieno[3,2-d]pyrimidin-6-yl)methanol (1.2 g, 4.013 mmol, 1 eq) in a (1:1) mixture of 1,4-dioxane: toluene (20 mL), is added 3-(m-tolyl)-1H-pyrazole (0.634 g, 4.013 mmol, 1 eq), and K₃PO₄ (1.7 g, 8.026 mmol, 2 eq). The reaction mixture is degassed for 10 min under nitrogen followed by the addition of Pd₂(dba)₃ (0.37 g, 0.401 mmol, 0.1 eq) and t-bu-XPhos (0.802 g, 0.802 mmol, 0.2 eq) at room temperature. The reaction mixture temperature is raised to 100° C. and stirred for 18 h. The progress of the reaction is monitored by TLC. After completion, the reaction mixture is cooled to room temperature and filtered through a Celite pad, washed with ethyl acetate, and the filtrate is evaporated under reduced pressure to obtain a brown gum (1.2 g). The crude product is purified by silica gel column (60-120) eluting with 30-35% ethyl acetate in hexanes to afford the title compound (240 mg; 15%) as an off white solid. Mass [m/z] 422.2 [M+H]⁺; ¹H-NMR (400 MHz, CDCl₃): 8.64 (d, J=2.4 Hz, 1H), 7.86 (s, 1H), 7.80 (d, J=7.6 Hz, 1H), 7.34 (m, 1H), 7.18 (d, J=8.0 Hz, 1H), 6.80 (d, J=2.8 Hz, 1H), 4.88 (d, J=6.0 Hz, 2H), 4.08 (m, 4H), 3.90 (m, 4H), 2.99 (m, 1H), 2.43 (s, 3H), 2.29 (s, 3H).

### Example 54

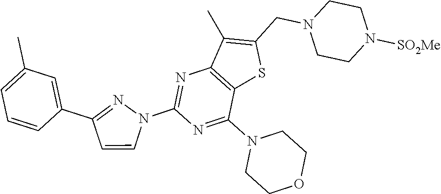 [SMILES: Cc1cccc(-c2ccn(-c3nc(N4CCOCC4)c4sc(CN5CCN(S(C)(=O)=O)CC5)c(C)c4n3)n2)c1]

### Step 8: 4-(6-(chloromethyl)-7-methyl-2-(3-(m-tolyl)-1H-pyrazol-1-yl)thieno[3,2-d]pyrimidin-4-yl)morpholine

Continuing the synthetic scheme from Example 53, to a solution of 7-methyl-4-morpholino-2-(3-(m-tolyl)-1H-pyrazol-1-yl)thieno[3,2-d]pyrimidin-6-yl)methanol (70 mg, 0.16 mmol, 1 eq) in DCM (5 mL) is added thionyl chloride (0.2 mL) at 0° C. The reaction mixture is stirred at 0° C. for 2 h. The progress of the reaction is monitored by TLC. After complete consumption of starting material, the reaction mixture is concentrated in vacuo to obtain a crude compound which is triturated with n-pentane to afford the title compound (65 mg; 89%) as a pale-yellow solid. Mass [m/z] 440.2 [M+H]⁺; ¹H-NMR (400 MHz, CDCl₃): 8.68 (d, J=6.0 Hz, 1H), 7.89 (s, 1H), 7.84 (d, J=6.0 Hz, 1H), 7.34 (m, 1H), 7.20 (d, J=7.2 Hz, 1H), 6.84 (s, 1H), 4.84 (s, 2H), 4.16 (br s, 4H), 3.93 (br s, 4H), 2.59 (s, 3H), 2.43 (s, 3H).

### Step 9: 4-(7-methyl-6-((4-(methylsulfonyl)piperazin-1-yl)methyl)-2-(3-(m-tolyl)-1H-pyrazol-1-yl)thieno[3,2-d]pyrimidin-4-yl)morpholine

To a stirred solution of 4-(6-(chloromethyl)-7-methyl-2-(3-(m-tolyl)-1H-pyrazol-1-yl)thieno[3,2-d]pyrimidin-4-yl)morpholine (40 mg) in IPA (4 mL) is added 1-(methylsulfonyl)piperazine (30 mg, 2 eq.) and DIPEA (5 eq) at RT. The reaction temperature is raised to 80° C. and stirred for 16 to 24 h. Progress of the reaction is monitored by TLC. After complete consumption of the starting material, the reaction mixture is brought to room temperature, diluted with ethyl acetate and washed with water. The organic layers are dried over Na₂SO₄, filtered and concentrated to obtain crude product (50 mg) as a brown solid. The crude material is purified by RP flash column chromatography (C18-6 g RP column), the product eluting at 75% acetonitrile/water. Product containing fractions are combined and lyophilized to afford the title compound (14 mg; 27%) as an off white solid. Mass [m/z] 568.2 [M+H]⁺; ¹H-NMR (400 MHz, DMSO-d₆): 8.75 (d, J=2.8 Hz, 1H), 7.76 (m, 2H), 7.35 (m, 1H), 7.19 (d, J=7.2 Hz, 1H), 7.02 (d, J=2.8 Hz, 1H)), 4.00 (t, 4H), 3.90 (s, 2H), 3.80 (t, 4H), 3.15 (s, 4H), 2.90 (s, 3H), 2.61 (s, 4H), 2.38 (d, J=8.0 Hz, 6H).

### Example 55

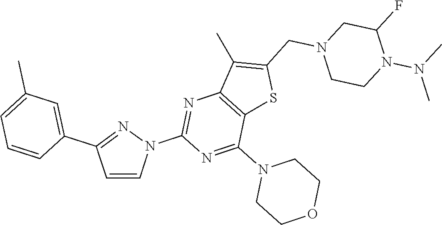 [SMILES: Cc1cccc(-c2ccn(-c3nc(N4CCOCC4)c4sc(CN5CCN(N(C)C)C(F)C5)c(C)c4n3)n2)c1]

### Step 9: 3-fluoro-N,N-dimethyl-1-((7-methyl-4-morpholino-2-(3-(m-tolyl)-1H-pyrazol-1-yl)thieno[3,2-d]pyrimidin-6-yl)methyl)piperidin-4-amine

Following the procedure according to Example 54, 4-(6-(chloromethyl)-7-methyl-2-(3-(m-tolyl)-1H-pyrazol-1-yl)thieno[3,2-d]pyrimidin-4-yl)morpholine (70 mg) is reacted with 4-(dimethylamino)-3-fluoropiperidine (47 mg) to provide a crude solid which is purified by RP flash column chromatography (C18-6 g), eluting at 82% acetonitrile/water to afford the title compound (11 mg; 12%) as an off white solid. Mass [m/z] 550.3 [M+H]⁺; ¹H-NMR (400 MHz, DMSO-d₆): 8.74 (d, J=2.4 Hz, 1H), 7.76 (m, 2H), 7.35 (m, 1H), 7.19 (d, J=7.6 Hz, 1H), 7.02 (d, J=2.8 Hz, 1H)), 4.98 (s, 1H), 4.00 (t, 5H), 3.87 (d, J=14.8 Hz, 3H), 3.80 (t, 4H), 3.17 (s, 1H), 2.98 (d, J=10.4 Hz, 1H), 2.37 (d, J=14.0 Hz, 6H), 2.26 (d, J=10.8 Hz, 6H), 2.21-2.15 (m, 1H), 1.80-1.76 (m, 1H), 1.65-1.63 (m, 1H).

### Example 56

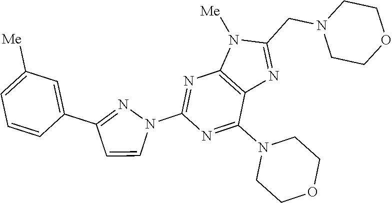 [SMILES: Cc1cccc(-c2ccn(-c3nc(N4CCOCC4)c4nc(CN5CCOCC5)n(C)c4n3)n2)c1]

### Step 1: 2,6-dichloro-9-(tetrahydro-2H-pyran-2-yl)-9H-purine

To a solution of 2,6-dichloro-9H-purine (10 g, 52.91 mmol, 1 eq) in ethyl acetate (10 vol), is added PTSA (91 mg, 0.5291 mmol, 0.01 eq) at room temperature, then DHP (15.4 mL, 84.65 mmol, 1.6 eq) is added. The resulting reaction mixture is stirred at room temperature for 18 h. The progress of the reaction is monitored by TLC. After complete consumption of starting material, the reaction mixture is concentrated under reduced pressure to obtain a solid (12 g), which is triturated with 20% ethyl acetate in hexane to afford the title compound (6.5 g; 46%) as an off-white solid. Mass [m/z] 473 [M+H]⁺; ¹H-NMR (400 MHz, CDCl₃): 8.32 (s, 1H), 5.76 (dd, J=2.4, 10.8 Hz, 1H), 4.21-4.19 (m, 1H), 3.81-3.71 (m, 1H), 2.18-2.15 (m, 1H), 2.10-2.07 (m, 1H), 1.98-1.95 (m, 1H), 1.83-1.79 (m, 2H), 1.78-1.72 (m, 1H).

### Step 2: 4-(2-chloro-9-(tetrahydro-2H-pyran-2-yl)-9H-purin-6-yl)morpholine

To a solution of 2,6-dichloro-9-(tetrahydro-2H-pyran-2-yl)-9H-purine (5 g, 18.38 mmol, 1 eq) in MeOH (30 mL) is added morpholine (1.6 g, 18.38 mmol, 1 eq) at 0° C. The reaction mixture is brought to room temperature and stirred for 4 h. The progress of the reaction is monitored by TLC. After completion, the reaction mixture is filtered, washed with water (1 mL) and dried to afford the title compound (3.5 g; 58%) as an off white solid. Mass [m/z] 324.2 [M−H]⁺; ¹H-NMR (400 MHz, CDCl₃): 7.91 (s, 1H), 5.70 (dd, J=2.0, 10.8 Hz, 1H), 4.29 (s, 3H), 4.16-4.12 (m, 1H), 3.82-3.79 (m, 5H), 3.76-3.73 (m, 1H), 2.99-1.91 (m, 2H), 1.90-1.79 (m, 1H), 1.78-1.71 (m, 2H), 1.70-1.63 (m, 1H).

### Step 3: 2-chloro-6-morpholino-9-(tetrahydro-2H-pyran-2-yl)-9H-purine-8-carbaldehyde

To a solution of 4-(2-chloro-9-(tetrahydro-2H-pyran-2-yl)-9H-purin-6-yl)morpholine (1.8 g, 5.57 mmol, 1 eq) in THE (20 mL) is added n-BuLi (2.5 M in hexane) (4.5 mL, 11.14 mmol, 2 eq) at −78° C. and the solution is stirred at −78° C. for 1 h, then DMF (2.5 mL, 33.43 mmol, 6 eq) is added at −78° C. and the reaction mixture is stirred at same temperature for 3 h. The progress of the reaction is monitored by TLC. After complete consumption of starting material, the reaction mixture is quenched with saturated ammonium chloride solution (50 mL), and extracted with ethyl acetate (20 mL×2). The combined organic layer is washed with brine solution (20 mL), dried over Na₂SO₄, filtered, and concentrated to obtain brown solid (2.1 g), which is triturated with diethyl ether to afford the title compound (1.2 g; 63%) as a yellow solid. Mass [m/z] 352 [M+H]⁺; ¹H-NMR (400 MHz, CDCl₃): 9.95 (s, 1H), 6.24 (dd, J=2.4, 11.2 Hz, 1H), 4.67 (s, 2H), 4.18-4.11 (m, 1H), 4.04 (s, 2H), 3.85-3.80 (m, 4H), 3.78-3.71 (m, 1H), 2.78-2.74 (m, 1H), 2.09-1.87 (m, 1H), 1.87-1.85 (m, 1H), 1.84-1.74 (m, 2H), 1.75-1.71 (m, 1H).

### Step 4: (2-chloro-6-morpholino-9-(tetrahydro-2H-pyran-2-yl)-9H-purin-8-yl)methanol

To a solution of 2-chloro-6-morpholino-9-(tetrahydro-2H-pyran-2-yl)-9H-purine-8-carbaldehyde (1.2 g, 3.41 mmol, 1 eq) in THF (10 mL) is added NaBH₄ (260 mg, 6.83 mmol, 2 eq) at 0° C., and the reaction temperature is raised to RT and stirred for 5 h. The progress of the reaction is monitored by TLC. After complete consumption of starting material, the reaction mixture is diluted with ethyl acetate (20 mL) and washed with water (10 mL). The organic layer is dried over Na₂SO₄, filtered, and concentrated to obtain a brown solid, which is triturated with n-pentane to afford the title compound (1 g; 83%) as a white solid. Mass [m/z] 554.1 [M+H]⁺; ¹H-NMR (400 MHz, DMSO-d₆): 5.70-5.66 (m, 2H), 4.74-4.65 (m, 2H), 4.17-4.02 (m, 5H), 3.72-3.64 (m, 5H), 2.66-2.55 (m, 2H), 1.77 (d, J=11.6 Hz, 1H), 1.69-1.55 (m, 3H).

### Step 5: (2-chloro-6-morpholino-9H-purin-8-yl)methanol

To a solution of (2-chloro-6-morpholino-9-(tetrahydro-2H-pyran-2-yl)-9H-purin-8-yl) methanol (800 mg, 2.26 mmol, 1 eq) in 1,4-dioxane (5 mL) is added 4N HCl in 1,4-dioxane (5 mL) at 0° C., and the reaction mixture temperature is raised to room temperature and stirred for 4 h. The progress of the reaction is monitored by TLC. After completion, the reaction mixture is concentrated under reduced pressure to obtain the title compound (600 mg; 98%) as a white solid. Mass [m/z] 270 [M−H]⁺; ¹H-NMR (400 MHz, DMSO-d₆): 4.84-4.70 (m, 4H), 4.58 (s, 2H), 4.11 (s, 3H), 3.70 (t, J=5.2 Hz, 4H), 3.56 (s, 2H).

### Step 6: 4-(8-(((tert-butyldimethylsilyl)oxy)methyl)-2-chloro-9H-purin-6-yl)morpholine

To a solution of (2-chloro-6-morpholino-9H-purin-8-yl)methanol (600 mg, 2.23 mmol, 1 eq) in DMF (5 mL) is added imidazole (455 mg, 6.69 mmol, 3 eq), and TBDMS-Cl (370 mg, 2.45 mmol, 1.1 eq) at 0° C., and the reaction temperature is raised to 25° C. and stirred for 12 h. The progress of the reaction is monitored by TLC. After completion, the reaction mixture is diluted with water (10 mL) and extracted with ethyl acetate (2×20 mL). The organic layer is dried over Na₂SO₄, filtered, and concentrated to obtain crude (600 mg) as an off white solid. The crude product is purified by silica gel column chromatography (60-120 Mesh) eluting with 15% ethyl acetate in hexane to afford the title compound (400 mg; 47%) as a white solid. Mass [m/z] 384.3 [M+H]⁺; ¹H-NMR (400 MHz, DMSO-d₆): 13.24 (s, 1H), 4.76 (s, 2H), 3.15 (br s, 4H), 3.70 (t, J=4.8 Hz, 4H), 0.86 (s, 9H), 0.75 (s, 6H).

### Step 7: 4-(8-(((tert-butyldimethylsilyl)oxy)methyl)-2-chloro-9-methyl-9H-purin-6-yl)morpholine)

To a solution of 4-(8-(((tert-butyldimethylsilyl)oxy)methyl)-2-chloro-9H-purin-6-yl)morpholine (400 mg, 1.04 mmol, 1 eq) in THF (5 mL) is added sodium hydride (60%) (90 mg, 2.08 mmol, 2 eq) at 0° C. and stirred for 30 minutes. Then methyl iodide (1 ml, 1.56 mmol, 1.5 eq) is added at 0° C. The reaction temperature is raised to 25° C. and stirred for 16 h. The progress of the reaction is monitored by TLC. After completion, the reaction mixture is quenched with water (10 mL) and extracted with ethyl acetate (2×20 mL). The combined organic layers are washed with brine solution (10 mL) and dried over Na₂SO₄, filtered and concentrated to obtain crude solid (400 mg), which is further purified by silica gel column chromatography (60-120 mesh), eluting with 15% ethyl acetate in hexane to afford the title compound (300 mg; 72%) as a white solid. Mass [m/z] 208 [M+H]⁺; ¹H-NMR (400 MHz, DMSO-d₆): 4.86 (s, 2H), 4.19 (s, 4H), 3.71 (d, J=4.8 Hz, 4H), 3.69 (s, 3H), 0.86 (s, 9H), 0.76 (s, 6H).

### Step 8: (2-chloro-9-methyl-6-morpholino-9H-purin-8-yl)methanol

To a solution of 4-(8-(((tert-butyldimethylsilyl)oxy)methyl)-2-chloro-9-methyl-9H-purin-6-yl)morpholine (600 mg, 1.50 mmol, 1 eq) in THE (10 mL) is added TBAF (1 M in THF, 3 mL, 3.01 mmol, 2 eq) at 0° C., then the reaction temperature is raised to 25° C. and stirred for 4 h. The progress of the reaction is monitored by TLC. After completion, the reaction mixture is quenched with water (10 mL) and extracted with ethyl acetate (20 mL). The organic layer is washed with brine solution (10 mL) and dried over Na₂SO₄, filtered and concentrated to afford the title compound (350 mg; 82%) as a white solid. Mass [m/z]284.1 [M+H]⁺; ¹H-NMR (400 MHz, DMSO-d₆): 5.64 (t, J=6.0 Hz, 1H), 4.65 (d, J=6.0 Hz, 2H), 3.80-4.30 (m, 4H), 3.72-3.70 (m, 7H).

### Step 9: (9-methyl-6-morpholino-2-(3-(m-tolyl)-1H-pyrazol-1-yl)-9H-purin-8-yl)methanol

To a solution of (2-chloro-9-methyl-6-morpholino-9H-purin-8-yl)methanol (350 mg, 1.23 mmol, 1 eq) in 1,4-dioxane:toluene (4:1) (10 mL) is added 3-(m-tolyl)-1H-pyrazole (200 mg, 1.23 mmol, 1 eq), and K₃PO₄ (530 mg, 2.47 mmol, 2 eq). The resulting reaction mixture is degassed for 10 minutes under nitrogen, followed by addition of Pd₂(dba)₃ (226 mg, 0.24 mmol, 0.2 eq) and t-Bu-XPhos (0.210 g, 0.49 mmol, 0.4 eq) at room temperature. Then the reaction temperature is raised to 100° C. and stirred for 18 h. After the reaction is complete, it is cooled to room temperature, filtered through a Celite pad and washed with ethyl acetate (10 mL). The filtrate is concentrated to obtain a crude product as a brown gummy liquid (400 mg). The crude product is purified by silica gel column chromatography (60-120) eluting with 5% MeOH in DCM to afford the title compound (145 mg; 24%) as a yellow solid. Mass [m/z] 406.2 [M+H]⁺; ¹H-NMR (400 MHz, DMSO-d₆): 8.72 (d, J=2.8 Hz, 1H), 7.78 (s, 1H), 7.74 (d, J=7.6 Hz, 1H), 7.35 (t, J=7.6 Hz, 1H), 7.19 (d, J=8.0 Hz, 1H), 7.08 (d, J=2.4 Hz, 1H), 5.64 (t, J=5.60 Hz, 1H), 4.58 (d, J=5.2 Hz, 1H), 4.29 (s, 3H), 3.81 (s, 3H), 3.78-3.71 (m, 4H), 2.39 (s, 3H), 1.98 (s, 1H).

### Step 10: 4-(8-(chloromethyl)-9-methyl-2-(3-(m-tolyl)-1H-pyrazol-1-yl)-9H-purin-6-yl)morpholine

To a solution of (9-methyl-6-morpholino-2-(3-(m-tolyl)-1H-pyrazol-1-yl)-9H-purin-8-yl)methanol (100 mg, 0.17 mmol, 1 eq) in DCM (5 mL) is added SOCl₂ (0.2 mL) at 0° C., and the reaction temperature is raised to 25° C. and stirred for 2 h. The progress of the reaction is monitored by TLC. After completion, the reaction mixture is concentrated under reduced pressure to obtain a solid (100 mg) which is triturated with n-pentane to afford the title compound (90 mg; 90%) as an off white solid. Mass [m/z] 424.2 [M+H]⁺; ¹H-NMR (400 MHz, DMSO-d₆): 8.73 (d, J=2.8 Hz, 1H), 7.78 (s, 1H), 7.74 (d, J=7.6 Hz, 1H), 7.35 (t, J=7.6 Hz, 1H), 7.18 (d, J=7.6 Hz, 1H), 7.02 (d, J=2.4 Hz, 1H), 5.08 (s, 2H), 4.29 (m, 4H), 3.84 (s, 3H), 3.77 (t, J=4.4 Hz, 4H), 2.39 (s, 3H).

### Step 11: 4-((9-methyl-6-morpholino-2-(3-(m-tolyl)-1H-pyrazol-1-yl)-9H-purin-8-yl)methyl)morpholine

To a solution of 4-(8-(chloromethyl)-9-methyl-2-(3-(m-tolyl)-1H-pyrazol-1-yl)-9H-purin-6-yl)morpholine (30 mg, 1 eq) in IPA (3 mL) is added morpholine (12 mg, 2 eq.) and DIPEA (3 eq) at room temperature, then the reaction mixture is heated to 800 for 16 h. The reaction mixture is quenched with cold water (5 mL) and extracted with ethyl acetate (2×15 mL). The organic layers are combined and dried over Na₂SO₄, filtered and concentrated to obtain a crude (32 mg) which is purified by Flash RP chromatography (C18, 6 g column) using water/acetonitrile as solvent system. The product elutes at 65-70% acetonitrile/water and after lyophilization affords the title product (10 mg; 30%) as a white solid. Mass [m/z] 475.3 [M+H]⁺; ¹H-NMR (400 MHz, DMSO-d₆): 8.70 (d, J=2.4 Hz, 1H), 7.77 (s, 1H), 7.74 (d, J=8.0 Hz, 1H), 7.35 (t, J=7.6 Hz, 1H), 7.19 (d, J=7.6 Hz, 1H), 6.99 (d, J=2.8 Hz, 1H), 4.27 (br s, 4H), 3.84 (s, 3H), 3.78-3.76 (m, 6H), 3.58 (br s, 4H), 2.45-2.44 (m, 4H), 2.39 (s, 3H).

### Example 57

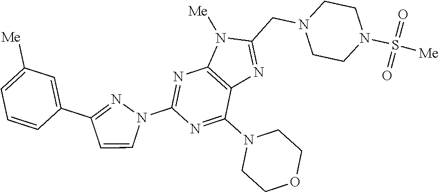 [SMILES: Cc1cccc(-c2ccn(-c3nc(N4CCOCC4)c4nc(CN5CCN(S(C)(=O)=O)CC5)n(C)c4n3)n2)c1]

### Step 11: 4-(9-methyl-8-((4-(methylsulfonyl)piperazin-1-yl)methyl)-2-(3-(m-tolyl)-1H-pyrazol-1-yl)-9H-purin-6-yl)morpholine

According to the procedure of Example 56, 4-(8-(chloromethyl)-9-methyl-2-(3-(m-tolyl)-1H-pyrazol-1-yl)-9H-purin-6-yl)morpholine (30 mg) is treated with 1-(methylsulfonyl)piperazine (23 mg) and the obtained crude (36 mg) is purified by Flash RP chromatography (C18, 6 g column) using water/acetonitrile as solvent system. The product elutes at 60% acetonitrile/water to afford the title compound (10 mg; 30%) as a white solid. Mass [m/z] 552.2 [M+H]⁺; ¹H-NMR (400 MHz, DMSO-d₆): 8.70 (d, J=2.8 Hz, 1H), 7.75 (t, J=8.8 Hz, 2H), 7.35 (t, J=7.6 Hz, 1H), 7.19 (d, J=7.6 Hz, 1H), 6.99 (d, J=2.4 Hz, 1H), 4.28 (s, 4H), 3.83 (s, 5H), 3.78-3.76 (m, 4H), 3.12 (s, 4H), 2.86 (s, 3H), 2.56-2.55 (m, 4H), 2.39 (s, 3H).

### Example 58

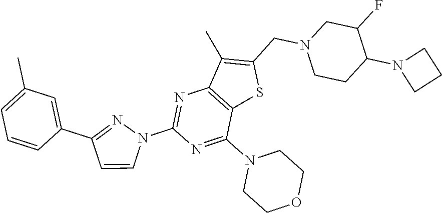 [SMILES: Cc1cccc(-c2ccn(-c3nc(N4CCOCC4)c4sc(CN5CCC(N6CCC6)C(F)C5)c(C)c4n3)n2)c1]

### Step 9: 4-(6-((4-(azetidin-1-yl)-3-fluoropiperidin-1-yl)methyl)-7-methyl-2-(3-(m-tolyl)-1H-pyrazol-1-yl)thieno[3,2-d]pyrimidin-4-yl)morpholine

Following the procedure according to Example 54, 4-(6-(chloromethyl)-7-methyl-2-(3-(m-tolyl)-1H-pyrazol-1-yl)thieno[3,2-d]pyrimidin-4-yl) (75 mg) is reacted with 4-(1-azetidinyl)-3-fluoropiperidine (54 mg) and the resulting solid is purified by RP flash column chromatography (C18-6 g) eluting at 68% acetonitrile/water to afford the title compound (8 mg; 8%) as an off white solid. Mass [m/z] 562.2 [M+H]⁺; ¹H-NMR (400 MHz, DMSO-d₆): 8.68 (d, J=2.8 Hz, 1H), 8.05 (t, J=5.6 Hz, 1H), 7.82-7.69 (m, 2H), 7.49 (m, 1H), 7.41 (t, J=8.0 Hz, 1H), 7.38-7.33 (m, 1H), 7.21 (d, J=7.6 Hz, 1H), 7.02 (d, J=2.8 Hz, 1H), 4.18 (t, J=6.8 Hz, 2H), 3.83-3.80 (br s, 4H), 3.68-3.62 (br s, 5H), 3.57-3.43 (m, 2H), 2.37 (s, 3H), 1.99 (s, 3H).

### Example 59

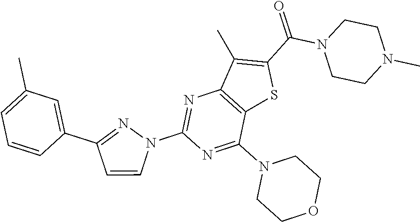 [SMILES: Cc1cccc(-c2ccn(-c3nc(N4CCOCC4)c4sc(C(=O)N5CCN(C)CC5)c(C)c4n3)n2)c1]

### Step 8: 7-methyl-4-morpholino-2-(3-(m-tolyl)-1H-pyrazol-1-yl)thieno[3,2-d]pyrimidine-6-carboxylic acid

Continuing the synthetic scheme from Example 53, to a solution of (7-methyl-4-morpholino-2-(3-(m-tolyl)-1H-pyrazol-1-yl)thieno[3,2-d]pyrimidin-6-yl)methanol (100 mg, 0.23 mmol, 1 eq) in acetone (5 mL) is added Jones reagent (3 mL) at 0°. The reaction temperature is raised to 25° C. and stirred for 2 h. The progress of the reaction is monitored by TLC. After completion of the reaction, it is quenched with saturated Na₂S203 solution (10 mL) and diluted with ethyl acetate and the organic phase is washed with brine solution (10 mL) and dried over Na₂SO₄, filtered and concentrated to afford the title compound (75 mg; 75%) as a brown solid. This is used without any further purification. Mass [m/z] 436.2 [M+H]⁺; ¹H-NMR (400 MHz, DMSO-d₆): 13.79 (br s, 1H), 8.78 (d, J=2.4 Hz, 1H), 7.77 (m, 2H), 7.36 (m, 1H), 7.20 (d, J=7.2 Hz, 1H), 7.05 (d, J=2.4 Hz, 1H), 4.06 (t, J=4.4 Hz, 4H), 3.81 (t, J=4.8 Hz, 4H), 2.70 (s, 3H), 2.39 (s, 3H).

### Step 9: (7-methyl-4-morpholino-2-(3-(m-tolyl)-1H-pyrazol-1-yl)thieno[3,2-d]pyrimidin-6-yl)(4-methylpiperazin-1-yl)methanone

To a solution of 7-methyl-4-morpholino-2-(3-(m-tolyl)-1H-pyrazol-1-yl)thieno[3,2-d]pyrimidine-6-carboxylic acid (30 mg, 1 eq) in DMF (5 mL) is added 1-methylpiperazine (2 eq), DIPEA (3 eq), and HATU (1.5 eq) at RT. The resulting reaction mixture is stirred at 25° C. for 16 h, and the progress of the reaction is monitored by TLC. After complete consumption of starting material, the reaction mixture is quenched with cold water and extracted with ethyl acetate. The organic layers are dried over Na₂SO₄, filtered and concentrated to obtain a crude product (34 mg) which is purified by RP flash column chromatography (C18-6 g), the product eluting at 75-80% acetonitrile/water. Product fractions are lyophilized, to afford the title product (15 mg; 50%) as an off white solid. Mass [m/z] 518.2 [M+H]⁺; ¹H-NMR (400 MHz, DMSO-d₆): 8.77 (d, J=2.8 Hz, 1H), 7.77 (t, J=5.6 Hz, 1H), 7.36 (t, J=7.6 Hz, 1H), 7.20 (d, J=7.6 Hz, 1H), 7.04 (d, J=2.8 Hz, 1H), 4.02 (t, J=4.4 Hz, 4H), 3.80 (d, J=5.2 Hz, 4H), 3.62 (s, 4H), 2.39 (s, 3H), 2.36-2.32 (m, 7H), 2.2 (s, 3H).

### Example 60

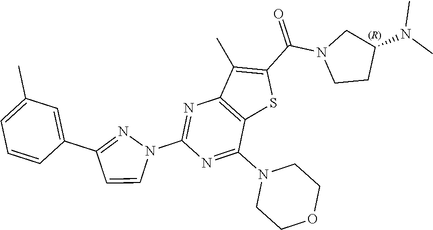

### Step 9: (R)-(3-(dimethylamino)pyrrolidin-1-yl)(7-methyl-4-morpholino-2-(3-(m-tolyl)-1H-pyrazol-1-yl)thieno[3,2-d]pyrimidin-6-yl)methanone

Following the procedure according to Example 59, 7-methyl-4-morpholino-2-(3-(m-tolyl)-1H-pyrazol-1-yl)thieno[3,2-d]pyrimidine-6-carboxylic acid (50 mg) and (R)—N,N-dimethylpyrrolidin-3-amine (2 eq.) are reacted to give a crude product (54 mg) which is purified by RP flash column chromatography (C18-6 g), eluting at 75-80% acetonitrile/water to afford the title compound (8 mg; 20%) as an off white solid. Mass [m/z] 532.2 [M+H]⁺; ¹H-NMR (400 MHz, DMSO-d₆): 8.77 (d, J=2.8 Hz, 1H), 7.77 (t, J=5.6 Hz, 2H), 7.36 (t, J=7.6 Hz, 1H), 7.20 (t, J=7.6 Hz, 1H), 7.04 (d, J=2.8 Hz, 1H), 4.03-4.01 (4H), 3.80 (t, J=4.8 Hz, 4H), 3.74-3.68 (m, 1H), 3.53-3.43 (m, 2H), 3.23-3.08 (m, 1H), 2.78-2.76 (br m, 1H), 2.39 (s, 6H), 2.19 (s, 3H), 2.10 (s, 3H), 1.75 (s, 1H), 1.23 (s, 2H).

### Example 61

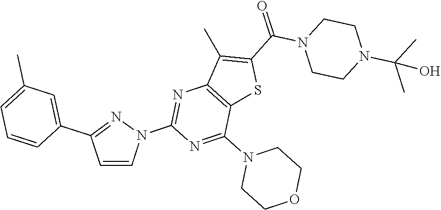 [SMILES: Cc1cccc(-c2ccn(-c3nc(N4CCOCC4)c4sc(C(=O)N5CCN(C(C)(C)O)CC5)c(C)c4n3)n2)c1]

### Step 9: (4-(2-hydroxypropan-2-yl)piperidin-1-yl)(7-methyl-4-morpholino-2-(3-(m-tolyl)-1H-pyrazol-1-yl)thieno[3,2-d]pyrimidin-6-yl)methanone

Following the procedure according to Example 59, 7-methyl-4-morpholino-2-(3-(m-tolyl)-1H-pyrazol-1-yl)thieno[3,2-d]pyrimidine-6-carboxylic acid is reacted with 2-(piperidin-4-yl)propan-2-ol (2 eq.) to give a crude solid (45 mg) which is purified by RP flash column chromatography (C18-6 g) eluting at 70-75% acetonitrile/water to afford the title compound (4 mg; 10%) as an off white solid. Mass [m/z] 560.2 [M+H]⁺; ¹H-NMR (400 MHz, DMSO-d₆): 8.77 (d, J=2.8 Hz, 1H), 8.05 (t, J=5.6 Hz, 1H), 7.82-7.69 (m, 2H), 7.49 (m, 1H), 7.41 (t, J=8.0 Hz, 1H), 7.38-7.33 (m, 1H), 7.21 (d, J=7.6 Hz, 1H), 7.02 (d, J=2.8 Hz, 1H), 4.18 (t, J=6.8 Hz, 2H), 3.83-3.80 (br s, 4H), 3.68-3.62 (br s, 5H), 3.57-3.43 (m, 2H), 2.37 (s, 3H), 1.99 (s, 3H).

### Example 62

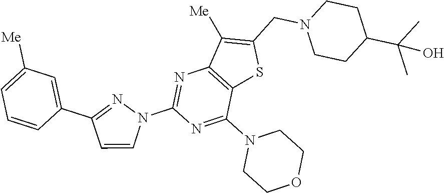 [SMILES: Cc1cccc(-c2ccn(-c3nc(N4CCOCC4)c4sc(CN5CCC(C(C)(C)O)CC5)c(C)c4n3)n2)c1]

### Step 11: 2-(1-((9-methyl-6-morpholino-2-(3-(m-tolyl)-1H-pyrazol-1-yl)-9H-purin-8-yl)methyl)piperidin-4-yl)propan-2-ol

According to the procedure of Example 56, 4-(8-(chloromethyl)-9-methyl-2-(3-(m-tolyl)-1H-pyrazol-1-yl)-9H-purin-6-yl)morpholine (50 mg) is treated with 2-(piperidin-4-yl)propan-2-ol (20 mg) and the obtained crude (64 mg) is purified by Flash RP chromatography (C18, 6 g column) using water/acetonitrile as solvent system. The product elutes at 60-70% acetonitrile/water to afford the title compound (11 mg; 17%) as a white solid. Mass [m/z] 531.4 [M+H]⁺; ¹H-NMR (400 MHz, DMSO-d₆): 8.71 (d, J=4.0 Hz, 1H), 7.77 (s, 1H), 7.74 (d, J=7.6 Hz, 1H), 7.35 (t, J=7.6 Hz, 1H), 7.19 (d, J=7.6 Hz, 1H), 7.0 (d, J=2.4 Hz, 1H), 4.27 (Brs, 4H), 4.01 (s, 1H), 3.82 (s, 3H), 3.77 (t, J=4.4 Hz, 4H), 3.70 (s, 2H), 3.13 (s, 1H), 2.87 (d, J=10.4 Hz, 2H), 2.39 (s, 3H), 1.97 (t, J=10.8 Hz, 2H), 1.65 (d, J=10.8 Hz, 2H), 1.26-1.12 (m, 4H), 1.01 (s, 6H).

### Example 63

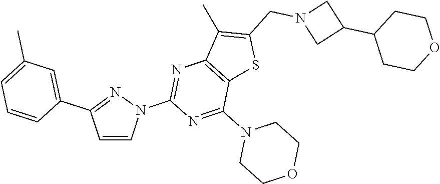 [SMILES: Cc1cccc(-c2ccn(-c3nc(N4CCOCC4)c4sc(CN5CC(C6CCOCC6)C5)c(C)c4n3)n2)c1]

### Step 9: 4-(7-methyl-6-((3-(tetrahydro-2H-pyran-4-yl)azetidin-1-yl)methyl)-2-(3-(m-tolyl)-1H-pyrazol-1-yl)thieno[3,2-d]pyrimidin-4-yl)morpholine

Following the procedure according to Example 54, 4-(6-(chloromethyl)-7-methyl-2-(3-(m-tolyl)-1H-pyrazol-1-yl)thieno[3,2-d]pyrimidin-4-yl)morpholine (75 mg) and 3-(tetrahydro-2H-pyran-4-yl)azetidine (48 mg) are reacted to give crude solid (80 mg) which is purified by RP flash column chromatography (C18-6 g) eluting at 55% acetonitrile/water to afford the title compound (23 mg; 46%) as an off white solid. Mass [m/z] 545.3 [M+H]⁺; ¹H-NMR (400 MHz, DMSO-d₆): 8.74 (d, J=2.8 Hz, 1H), 7.76 (t, J=6.0 Hz, 2H), 7.35 (t, J=7.6 Hz, 1H), 7.19 (d, J=7.6 Hz, 1H), 7.01 (d, J=2.8 Hz, 1H), 4.00 (t, J=4.4 Hz, 4H), 3.80 (s, 2H), 3.84-3.74 (m, 6H), 3.46 (t, J=7.6 Hz, 2H), 3.26 (t, J=10.0 Hz, 2H), 2.93 (t, J=7.2 Hz, 2H), 2.39 (s, 3H), 2.33-2.32 (m, 3H), 2.22-2.16 (m, 1H), 1.63 (d, J=9.6 Hz, 1H), 1.49 (d, J=12.8 Hz, 2H), 1.10-1.01 (m, 2H).

### Example 64

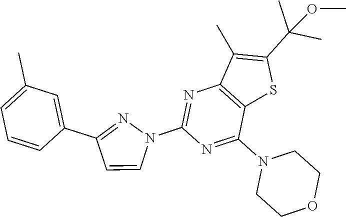 [SMILES: COC(C)(C)c1sc2c(N3CCOCC3)nc(-n3ccc(-c4cccc(C)c4)n3)nc2c1C]

### Step 1: 2-(2-chloro-7-methyl-4-morpholinothieno[3,2-d]pyrimidin-6-yl)propan-2-ol:#

To a solution of 4-(2-chloro-7-methylthieno[3,2-d]pyrimidin-4-yl)morpholine (200 mg, 0.74 mmol, 1 eq) in THE (4 mL) is added n-BuLi (2.5 M in hexane) (1 mL, 2.97 mmol, 4 eq) at −78° C. After stirring for 1 h at −78° C., acetone (0.3 mL, 2.97 mmol, 4 eq) is added and the reaction mixture is stirred at the same temperature for 3 h. The reaction mixture is quenched with saturated ammonium chloride solution (6 mL) and extracted with ethyl acetate (20 mL×2). The combined organic layers are washed with saturated brine solution (20 mL) and dried over Na₂SO₄, filtered and concentrated to obtain a brown gummy liquid (210 mg). The crude compound is purified by flash chromatography eluting with 25-30% of ethyl acetate in hexane, to afford the title compound (100 mg; 41%) as a pale-yellow solid. Mass [m/z] 318.1 [M+H]⁺; ¹H-NMR (400 MHz, DMSO-d₆): 6.01 (s, 1H), 3.89 (t, J=4.4 Hz, 4H), 3.73 (t, J=4.8 Hz, 4H), 2.35 (s, 3H), 1.57 (s, 6H).

### Step 2: 4-(2-chloro-6-(2-methoxypropan-2-yl)-7-methylthieno[3,2-d]pyrimidin-4-yl)morpholine

To a solution of 2-(2-chloro-7-methyl-4-morpholinothieno[3,2-d]pyrimidin-6-yl)propan-2-ol (50 mg, 0.15 mmol, 1 eq) in THE (5 mL) is added sodium hydride (60%) (14 mg, 0.22 mmol, 1.5 eq) at 0° C. The reaction mixture is stirred at 0° C. for 30 min and then methyl iodide (64 mg, 0.30 mmol, 2 eq) is added at 0° C., and the reaction mixture is slowly brought to room temperature and stirred for 3 h. The progress of the reaction is monitored by TLC. After complete consumption of the starting material, the reaction mixture is concentrated to obtain a crude solid which is triturated with n-pentane to afford the title compound (45 mg; 86%) as a pale-yellow solid. Mass [m/z] 342.1 [M+H]⁺; #¹H-NMR (400 MHz, CDCl₃): 3.99 (t, J=6.4 Hz, 4H), 3.83 (t, J=5.2 Hz, 4H), 3.23 (s, 3H), 2.48 (s, 3H), 1.65 (s, 6H).

### Step 3: 4-(6-(2-methoxypropan-2-yl)-7-methyl-2-(3-(m-tolyl)-1H-pyrazol-1-yl)thieno[3,2-d]pyrimidin-4-yl)morpholine

To a solution of 4-(2-chloro-6-(2-methoxypropan-2-yl)-7-methylthieno[3,2-d]pyrimidin-4-yl)morpholine (50 mg, 0.14 mmol, 1 eq) and 3-(m-tolyl)-1H-pyrazole (23 mg, 0.14 mmol, 1 eq) in 1,4-dioxane:toluene (5 mL) is added K₃PO₄ (28 mg, 0.29 mmol, 2 eq), and the resulting solution is degassed for 20 minutes. Pd₂(dba)₃ (14 mg, 0.01 mmol, 0.1 eq), and t-bu-XPhos (12 mg, 0.02 mmol, 0.2 eq) are added at room temperature, and then the reaction mixture is stirred at 100° C. for 16 h. The progress of the reaction is monitored by TLC. After completion, the reaction mixture is filtered through a Celite pad and washed with ethyl acetate (10 mL). The filtrate is concentrated under reduced pressure to obtain a brown gummy liquid (50 mg) which is purified by Prep HPLC (Column: Gemini C18, 250×21.2 mm, 5 μm; Mobile phase: [ACN: 0.1% of Formic acid in Water]; time/B %: 0/40, 14/55, 25/90, 26/95) to afford the title compound (8 mg; 11%) as an off-white solid. Mass [m/z] 464.3[M+H]⁺; ¹H-NMR (400 MHz, DMSO-d₆): 8.75 (d, J=2.8 Hz, 1H), 7.76 (t, J=4.0 Hz, 2H) 7.35 (t, J=7.6 Hz, 1H), 7.19 (d, J=7.2 Hz, 1H), 7.01 (d, J 10=2.4 Hz, 1H), 4.02 (t, J=4.4 Hz, 4H), 3.80 (t, J=4.8 Hz, 4H), 3.17 (s, 3H), 2.50-2.49 (m, 3H), 2.39 (s, 3H), 1.63 (s, 6H).

### Example 65

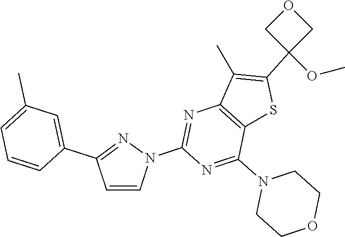 [SMILES: COC1(c2sc3c(N4CCOCC4)nc(-n4ccc(-c5cccc(C)c5)n4)nc3c2C)COC1]

### Step 1: (3-(2-chloro-7-methyl-4-morpholinothieno[3,2-d]pyrimidin-6-yl)oxetan-3-ol

To a solution of 4-(2-chloro-7-methylthieno[3,2-d]pyrimidin-4-yl)morpholine (200 mg, 0.74 mmol, 1 eq) in THE (5 mL) is added n-BuLi (2.5 M in hexane) (1 mL, 2.97 mmol, 4 eq) at −78° C., and the solution is stirred for 1 h, then 3-oxetanone (212 mg, 2.97 mmol, 4 eq) is added, and the reaction mixture is stirred at same temperature for another 3 h. The progress of the reaction is monitored by TLC. After completion, the reaction mixture is quenched with saturated ammonium chloride solution (10 mL), and extracted with ethyl acetate (20 mL×2), and washed with saturated brine solution (20 mL). The organic layers are dried with Na₂SO₄, filtered and concentrated to obtain a brown gummy liquid (210 mg) which is purified by flash chromatography, eluting at 20-25% ethyl acetate in hexane, to afford the title compound (150 mg; 59%) as a pale-yellow solid. Mass [m/z]342.0 [M+H]⁺; #¹H-NMR (400 MHz, DMSO-d₆): 7.13 (s, 1H), 4.94 (d, J=7.2 Hz, 2H), 4.85 (d, J=7.2 Hz, 2H), 3.90 (t, J=4.4 Hz, 4H), 3.74 (t, J=5.2 Hz, 4H), 2.43 (s, 3H).

### Step 2: 4-(2-chloro-6-(3-methoxyoxetan-3-yl)-7-methylthieno[3,2-d]pyrimidin-4-yl)morpholine

To a solution of (3-(2-chloro-7-methyl-4-morpholinothieno[3,2-d]pyrimidin-6-yl)oxetan-3-ol (90 mg, 0.26 mmol, 1 eq) in THE (5 mL), is added sodium hydride (60%) (20 mg, 0.39 mmol, 1.5 eq) at 0° C. The reaction mixture is stirred at 0° C. for 30 min, then methyl iodide (75 mg, 0.52 mmol, 2 eq) is added at 0° C. and the reaction mixture is stirred at room temperature for 3 h. The progress of the reaction is monitored by TLC and, after completion of the reaction, it is quenched with ice cold water (10 mL) and extracted with ethyl acetate (2×20 mL). The organic layers are dried with Na₂SO₄ and concentrated to obtain a crude compound which is triturated with n-pentane to afford the title compound (65 mg; 69%) as pale brown solid. Mass [m/z] 356.1 [M+H]⁺; ¹H-NMR (400 MHz, CDCl₃): 5.0-4.95 (m, 4H), 4.01 (t, J=4.4 Hz, 4H), 3.84 (t, J=4.8 Hz, 4H), 3.17 (s, 3H), 2.36 (s, 3H).

### Step 3: 4-(6-(3-methoxyoxetan-3-yl)-7-methyl-2-(3-(m-tolyl)-1H-pyrazol-1-yl)thieno[3,2-d]pyrimidin-4-yl)morpholine

To a solution of 4-(2-chloro-6-(3-methoxyoxetan-3-yl)-7-methylthieno[3,2-d]pyrimidin-4-yl)morpholine (30 mg, 0.08 mmol, 1 eq) in THE (5 mL) is added sodium hydride (60%) (12 mg, 0.16 mmol, 2 eq) at 0° C. The reaction mixture is stirred at 0° C. for 20 minutes then 3-(m-tolyl)-1H-pyrazole (14 mg, 0.08 mmol, 1 eq) is added at 0° C. The temperature is raised to 100° C. for 16 h. The progress of the reaction is monitored by TLC. After completion, the reaction mixture is quenched with cold water (10 mL) and extracted with ethyl acetate (2×20 mL). The organic layers are dried over Na₂SO₄, filtered, and concentrated to obtain a crude solid, which is purified by Prep HPLC (Column: Gemini C18, 250×21.2 mm, 5 μm; Mobile phase: [ACN: 0.1% of Formic acid in Water]; time/B %: 0/40, 14/55, 25/90, 26/95) to afford the title compound (9.5 mg; 0.3%) as a white solid. Mass [m/z] 478.2 [M+H]⁺; ¹H-NMR (400 MHz, DMSO-d₆): 8.77 (d, J=2.8 Hz, 1H), 7.76 (d, J=12 Hz, 2H) 7.36 (t, J=7.6 Hz, 1H), 7.20 (d, J=7.6 Hz, 1H), 7.03 (d, J=2.8 Hz, 1H), 4.96-4.91 (m, 4H), 4.04 (t, J=4.8 Hz, 4H), 3.81 (t, J=5.2 Hz, 4H), 3.15 (s, 3H), 2.39 (s, 3H), 2.36 (s, 3H).

### Example 66

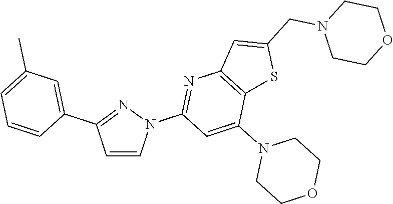 [SMILES: Cc1cccc(-c2ccn(-c3cc(N4CCOCC4)c4sc(CN5CCOCC5)cc4n3)n2)c1]

### Step 1: 4-(5-chlorothieno[3,2-b]pyridin-7-yl)morpholine

To a solution of 5,7-dichlorothieno[3,2-b]pyridine (3.0 g, 14.85 mmol, 1.0 eq.) in ethylene glycol (10 mL) at RT is added morpholine (2.5 mL, 29.7 mmol, 2.0 eq.) followed by TEA (4.0 mL, 29.7 mmol, 2.0 eq.). The reaction temperature is raised to 120° C. and stirred for 16 h. The progress of the reaction is monitored by TLC. After completion, it is diluted with water (30 mL) and the resulting solid is filtered, washed with water, and dried to obtain a brown solid (2.8 g), which is triturated with diethyl ether, filtered and dried to afford the title compound (2.5 g, 67%) as brown solid. Mass [m/z]255 [M+H]⁺; ¹H-NMR (400 MHz, CDCl₃): 7.65 (d, J=5.2 Hz, 1H), 7.44 (d, J=5.6 Hz, 1H), 6.65 (s, 1H), 3.92 (t, J=4.8 Hz, 4H), 3.47 (t, J=3.2 Hz, 4H).

### Step 2: 5-chloro-7-morpholinothieno[3,2-b]pyridine-2-carbaldehyde:#

To a stirred solution of dry THE (30 mL) is added n-BuLi (2.5 M in hexane) (12 mL, 29.52 mmol, 2.5 eq.), followed by a solution of 4-(5-chlorothieno[3,2-b]pyridin-7-yl)morpholine (3.0 g; 11.8 mmol, 1.0 eq.) dissolved in THE (10 mL) added dropwise at −78° C. and the resulting mixture is allowed to stir for 45 min at −78° C. Then DMF (5.7 mL, 70.8 mmol, 6.0 eq.) is added at −78° C., and the reaction is continued to stir for 20 min at −78° C. After completion of the reaction (monitored by TLC), the resulting mixture is allowed to warm to 0° C., it is diluted with water, and extracted with EtOAc (2×50 mL). The combined organic layers are washed with 1N HCl (50 mL), followed by brine (50 mL), dried over Na₂SO₄, filtered, and concentrated under reduced pressure to provide a crude product (2.7 g) as yellow solid. The crude product is triturated with diethyl ether and dried to afford the title compound # (1.7 g, 51%) as a yellow solid. Mass [m/z] 283 [M+H]⁺; ¹H-NMR (400 MHz, CDCl3): 10.16 (s, 1H), 8.07 (s, 1H), 6.74 (s, 1H), 3.93 (t, J=4.8 Hz, 4H), 3.49 (t, J=4.8 Hz, 4H).

### Step 3: 5-chloro-7-morpholinothieno[3,2-b]pyridin-2-yl)methanol

To a stirred solution of 5-chloro-7-morpholinothieno[3,2-b]pyridine-2-carbaldehyde (2.0 g, 7.06 mmol, 1.0 eq.) in a (1:1) mixture of MeOH:DCM (20 mL) is added NaBH₄ (0.54 g, 14.13 mmol, 2.0 eq.) at 0° C. The resulting mixture is allowed to stir at RT for 3 h. After completion of the reaction (monitored by TLC), it is diluted with water and extracted with EtOAc (2×50 mL). The combined organic layers are washed with brine (50 mL), dried over Na₂SO₄, filtered, and concentrated under reduced pressure to provide a crude product (2.0 g) as brown solid. The crude is triturated with diethyl ether and dried to afford the title compound (1.5 g, 71%) as white solid. Mass [m/z] 285.1 [M+H]⁺; ¹H-NMR (400 MHz, CDCl3): 7.28 (t, J=0.8 Hz, 1H), 6.62 (s, 1H), 4.96 (dd, J=0.8 Hz, 6.0 Hz, 2H), 3.91 (t, J=4.4 Hz, 4H), 3.45 (t, J=4.8 Hz, 4H), 2.23 (t, J=6.4 Hz, 1H).

### Step 4: 7-morpholino-5-(3-(m-tolyl)-1H-pyrazol-1-yl)thieno[3,2-b]pyridin-2-yl) methanol

To a stirred solution of 5-chloro-7-morpholinothieno[3,2-b]pyridin-2-yl)methanol (0.5 g, 1.75 mmol, 1.0 eq.) and 3-(m-tolyl)-1H-pyrazole (0.27 g, 1.75 mmol, 1.0 eq.) in (1:1) mixture of 1,4-dioxane: toluene (10 mL) is added K₃PO₄ (0.74 g, 3.50 mmol, 2.0 eq.). The reaction mixture is degassed for 15 min and Pd₂(dba)₃ (0.32 g, 0.35 mmol, 0.2 eq.) is added, followed by t-Bu-XPhos (0.3 g, 0.70 mmol, 0.4 eq.). The resulting reaction mixture is allowed to stir at 120° C. for 16 h. After completion of the reaction (monitored by TLC), it is diluted with EtOAc, dried, filtered, and concentrated. The resulting residue is purified by reverse phase flash purification (C18, 12 g column), eluting with 50% to 60% acetonitrile/water, to afford the title compound (0.2 g, 28%) as white solid. Mass [m/z] 407 [M+H]⁺; ¹H-NMR (400 MHz, CDCl3): 8.65 (d, J=2.4 Hz, 1H), 7.77 (s, 1H), 7.73 (d, J=8.0 Hz, 1H), 7.54 (s, 1H), 7.34 (s, 1H), 7.32 (s, 1H), 7.18 (d, J=7.6 Hz, 1H), 6.77 (d, J=2.8 Hz, 1H), 4.98 (dd, J=0.8 Hz, 6.0 Hz, 2H), 3.95 (t, J=4.8 Hz, 4H), 3.56 (t, J=4.8 Hz, 4H), 2.44 (s, 3H), 2.04 (t, J=6.0 Hz, 1H).

### Step 5: 4-(2-(chloromethyl)-5-(3-(m-tolyl)-1H-pyrazol-1-yl)thieno[3,2-b]pyridin-7-yl)morpholine

To a stirred solution of 7-morpholino-5-(3-(m-tolyl)-1H-pyrazol-1-yl)thieno[3,2-b]pyridin-2-yl)methanol (0.15 g, 0.36 mmol, 1.0 eq.) in DCM (5 mL) is added SOCl₂ (0.13 g, 1.10 mmol, 3.0 eq.) at 0° C. The reaction mixture is stirred at RT for 4 h. The progress of the reaction is monitored by TLC. After completion, the reaction mixture is concentrated under reduced pressure to obtain a crude product (0.17 g) which is triturated with n-pentane to afford the title compound (0.10 g, 64%) as an off white solid. Mass [m/z] 425 [M+H]⁺; ¹H-NMR (400 MHz, CDCl3): 9.74 (d, J=2.0 Hz, 1H), 8.45 (s, 1H), 7.71 (s, 2H), 7.56 (s, 1H), 7.36 (s, 1H), 7.23 (s, 1H), 6.90 (s, 1H), 4.84 (s, 2H), 3.87-4.00 (m, 8H), 2.45 (s, 3H).

### Step 6: 4-((7-morpholino-5-(3-(m-tolyl)-1H-pyrazol-1-yl)thieno[3,2-b]pyridin-2-yl)methyl)morpholine

To a stirred solution of 4-(2-(chloromethyl)-5-(3-(m-tolyl)-1H-pyrazol-1-yl)thieno[3,2-b]pyridin-7-yl)morpholine (50 mg, 1.0 eq.) in THE (10 vol) at room temperature is added morpholine (20.5 mg, 2.0 eq.), followed by K₂CO₃ (2.5 eq.). The reaction mixture temperature is heated to 80° C. and stirred for 16 h. The progress of the reaction is monitored by TLC. After completion, the reaction is diluted with water (10 vol) and extracted with ethyl acetate (2×10 vol). The combined organics are dried over anhydrous Na₂SO₄, filtered, and evaporated under reduced pressure to obtain a crude product, which is then triturated with diethyl ether (10 vol) to afford the title compound (12 mg; 24%) as an off-white solid. Mass [m/z] 476 [M+H]⁺; ¹H-NMR (400 MHz, DMSO-d₆): 8.67 (d, J=2.8 Hz, 1H), 7.79 (s, 1H), 7.76 (s, 1H), 7.43 (s, 1H), 7.38 (s, 1H), 7.35 (d, J=7.2 Hz, 1H), 7.20 (d, J=7.2 Hz, 1H), 7.04 (d, J=2.8 Hz, 1H), 3.86 (d, J=4.4 Hz, 4H), 3.83 (s, 2H), 3.62 (t, J=4.4 Hz, 4H), 3.50 (t, J=4.8 Hz, 4H), 2.49-2.51 (m, 4H), 2.39 (s, 3H).

### Example 67

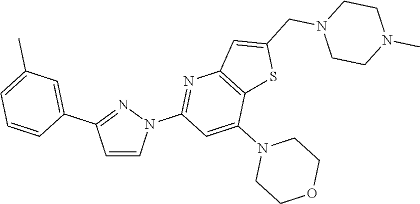 [SMILES: Cc1cccc(-c2ccn(-c3cc(N4CCOCC4)c4sc(CN5CCN(C)CC5)cc4n3)n2)c1]

### Step 6: 4-(2-((4-methylpiperazin-1-yl)methyl)-5-(3-(m-tolyl)-1H-pyrazol-1-yl)thieno[3,2-b]pyridin-7-ylmorpholine

According to the procedure of Example 66, 4-(2-(chloromethyl)-5-(3-(m-tolyl)-1H-pyrazol-1-yl)thieno[3,2-b]pyridin-7-yl)morpholine (50 mg) and 1-methylpiperazine (23.5 mg) are reacted to obtain a crude product which is then purified by Flash RP chromatography (C18, 12 g column), eluting with 30-40% acetonitrile/water, to afford the title compound (10 mg; 20%) as an off-white solid. Mass [m/z] 489 [M+H]⁺; ¹H-NMR (400 MHz, DMSO-d₆): 8.64 (d, J=2.4 Hz, 1H), 7.77 (s, 1H), 7.73 (d, J=7.6 Hz, 1H), 7.53 (s, 1H), 7.33 (t, J=7.6 Hz, 1H), 7.28 (s, 1H), 7.18 (d, J=6.8 Hz, 1H), 6.77 (d, J=2.4 Hz, 1H), 3.96 (t, J=8.4 Hz, 4H), 3.84 (s, 2H), 3.55 (t, J=4.8 Hz, 4H), 2.69 (s, 8H), 2.44 (s, 6H).

### Example 68

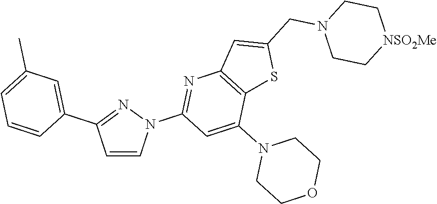

### Step 6: 4-(2-((4-(methylsulfonyl)piperazin-1-yl)methyl)-5-(3-(m-tolyl)-1H-pyrazol-1-yl)thieno[3,2-b]pyridin-7-yl)morpholine

According to the procedure of Example 66, 4-(2-(chloromethyl)-5-(3-(m-tolyl)-1H-pyrazol-1-yl)thieno[3,2-b]pyridin-7-yl)morpholine (50 mg) and 1-(methylsulfonyl) piperazine (38.5 mg) are reacted to obtain a crude product, which is then purified by Flash RP chromatography (C18, 12 g column), eluting with 30-40% acetonitrile/water, to afford the title compound (12 mg; 18%) as an off-white solid. Mass [m/z] 553 [M+H]⁺; ¹H-NMR (400 MHz, DMSO-d₆): 8.67 (d, J=2.8 Hz, 1H), 7.79 (s, 1H), 7.77 (s, 1H), 7.44 (s, 1H), 7.39 (s, 1H), 7.36 (s, 1H), 7.21 (s, 1H), 7.04 (d, J=2.4 Hz, 1H), 3.91 (s, 2H), 3.85 (t, J=4.0 Hz, 4H), 3.51 (t, J=4.4 Hz, 4H), 3.15 (s, 4H), 2.90 (s, 3H), 2.59 (s, 4H), 2.39 (s, 3H).

### Example 69

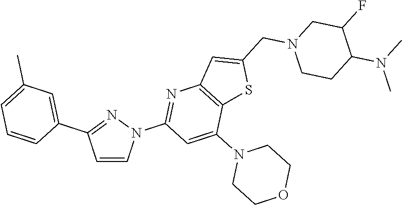 [SMILES: Cc1cccc(-c2ccn(-c3cc(N4CCOCC4)c4sc(CN5CCC(N(C)C)C(F)C5)cc4n3)n2)c1]

### Step 6: 3-fluoro-N,N-dimethyl-1-((7-morpholino-5-(3-(m-tolyl)-1H-pyrazol-1-yl)thieno[3,2-b]pyridin-2-yl)methyl)piperidin-4-amine

Following the first five steps of Example 66, to a stirred solution of 4-(2-(chloromethyl)-5-(3-(m-tolyl)-1H-pyrazol-1-yl)thieno[3,2-b]pyridin-7-yl)morpholine (100 mg, 1.0 eq.) in IPA (10 vol) at room temperature is added 3-fluoro-N,N-dimethyl-piperidin-4-amine (35.4 mg, 2 eq.), followed by DIPEA (2.0 eq). The reaction mixture is allowed to stir at 80° C. for 16 h. The progress of the reaction is monitored by TLC. After completion, it is concentrated under reduced pressure, diluted with water (10 vol), and extracted with EtOAc (2×10 vol). The combined organic layers are dried over anhydrous Na₂SO₄, filtered, and evaporated under reduced pressure to obtain a crude product, which is then purified by Flash RP chromatography (C18, 12 g column), eluting with 30-40% acetonitrile/water to afford the title compound (8 mg; 8%) as an off-white solid. Mass [m/z] 535 [M+H]⁺; ¹H-NMR (400 MHz, MeOD): 8.62 (d, J=2.4 Hz, 1H), 7.77 (s, 1H), 7.71 (d, J=7.6 Hz, 1H), 7.49 (s, 1H), 7.34 (s, 1H), 7.30-7.32 (m, 1H), 7.19 (d, J=7.6 Hz, 1H), 6.89 (d, J=2.4 Hz, 1H), 5.08 (s, 1H), 3.92-3.95 (m, 4H), 3.90 (s, 2H), 3.57 (t, J=4.8 Hz, 4H), 3.12-3.13 (m, 1H), 2.47 (s, 6H), 2.42 (s, 4H), 2.24-2.38 (m, 1H), 1.98-2.08 (m, 1H), 1.87-1.92 (m, 1H).

### Example 70

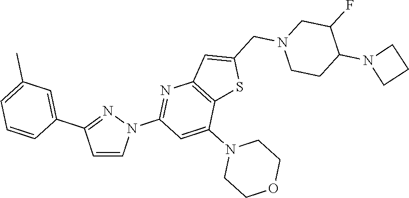 [SMILES: Cc1cccc(-c2ccn(-c3cc(N4CCOCC4)c4sc(CN5CCC(N6CCC6)C(F)C5)cc4n3)n2)c1]

### Step 6: 4-(2-((4-(azetidin-1-yl)-3-fluoropiperidin-1-yl)methyl)-5-(3-(m-tolyl)-1H-pyrazol-1-yl)thieno[3,2-b]pyridin-7-yl)morpholine

According to the procedure of Example 69, 4-(2-(chloromethyl)-5-(3-(m-tolyl)-1H-pyrazol-1-yl)thieno[3,2-b]pyridin-7-yl)morpholine (100 mg) and 4-(azetidin-1-yl)-3-fluoropiperidine (35.2 mg) are reacted to obtain a crude product, which is purified by Flash RP chromatography (C18, 12 g column), eluting with 50-55% acetonitrile/water, to afford the title compound (10 mg; 10%) as an off-white solid. Mass [m/z] 547 [M+H]⁺; ¹H-NMR (400 MHz, MeOD): 8.61 (d, J=2.8 Hz, 1H), 7.77 (s, 1H), 7.71 (d, J=8.0 Hz, 1H), 7.50 (s, 1H), 7.32 (t, J=7.2 Hz, 2H), 7.19 (d, J=7.6 Hz, 1H), 6.89 (d, J=2.4 Hz, 1H), 4.96 (s, 1H), 3.91-4.00 (m, 9H), 3.55-3.63 (m, 4H), 3.38 (d, J=11.2 Hz, 1H), 3.09 (d, J=13.2 Hz, 1H), 2.42 (s, 6H), 2.30 (t, J=9.6 Hz, 1H), 1.97 (s, 1H), 1.77-1.89 (m, 2H).

### Example 71

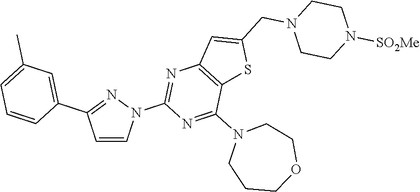 [SMILES: Cc1cccc(-c2ccn(-c3nc(N4CCCOCC4)c4sc(CN5CCN(S(C)(=O)=O)CC5)cc4n3)n2)c1]

### Step 1: 2,4-dichlorothieno[3,2-d]pyrimidine

A stirred solution of thieno[3,2-d]pyrimidine-2,4 (1H,3H)-dione (10 g, 59.52 mmol, 1 eq) in POCl₃ (60 mL, 6 vol) is heated at 100° C. for 16 h. The reaction solution is cooled to room temperature and evaporated in vacuo to provide a brown gummy residue which is diluted with ice cold water and stirred for 30 min, and the resulting precipitate is filtered and dried under vacuum to afford the title compound (10 g; 83%) as a pale brown solid. Mass m/z 205 [M+H]⁺; ¹H-NMR (400 MHz, DMSO-d₆): 8.71 (d, J=5.6 Hz, 1H), 7.751 (d, J=5.2 Hz, 1H).

### Step 2: 2-chloro-4-(methylthio)thieno[3,2-d]pyrimidine

To a solution of 2,4-dichlorothieno[3,2-d]pyrimidine (10 g, 49.01 mmol, 1 eq) in THE (20 mL) is added sodium thiomethoxide (3.4 g, 49.01 mmol, 1 eq) at 0° C., then the reaction is stirred at room temperature for 8 h. The reaction is then quenched with water (50 mL) and extracted with ethyl acetate (2×50 mL). The organic layer is dried over Na₂SO₄, filtered, and evaporated under reduced pressure to provide a crude solid which is triturated with n-pentane to afford the title compound (8.2 g; 78%) as an off white solid. Mass m/z 217 [M+H]⁺; ¹H-NMR (400 MHz, DMSO-d₆): 8.478 (d, J=5.2 Hz, 1H), 7.607 (d, J=5.6 Hz, 1H), 2.74 (s, 3H).

### Step 3: 2-chloro-4-(methylthio)thieno[3,2-d]pyrimidine-6-carbaldehyde

To a solution of 2-chloro-4-(methylthio)thieno[3,2-d]pyrimidine (6 g, 5.57 mmol, 1 eq) in dry THE (40 ml) is added n-BuLi (2.5 M in hexane) (20 mL, 11.15 mmol, 2 eq) at −78° C., and the solution is stirred for 1 h. DMF (2.61 mL, 33.4 mmol, 6 eq) is then added at −78° C. and the reaction mixture is stirred at the same temperature for an additional 2 h. The reaction is quenched with saturated ammonium chloride solution (35 mL) and extracted with ethyl acetate (2×50 mL). The combined organic layer is dried over Na₂SO₄, filtered, and evaporated under reduced pressure to provide a solid which is triturated with diethyl ether to afford the title compound (3.1 g; 67%) as a light brown solid. Mass m/z 245.1 [M+H]⁺; ¹H-NMR (400 MHz, DMSO-d₆): 10.25 (s, 1H), 8.49 (s, 1H), 2.77 (s, 3H).

### Step 4: 2,4-dichlorothieno[3,2-d]pyrimidine-6-carbaldehyde

To a solution of 2-chloro-4-(methylthio)thieno[3,2-d]pyrimidine-6-carbaldehyde (4 g, 16.39 mmol, 1 eq) in DCM (40 ml) is added SOCl₂ (20 mL, 32.78 mmol, 2 eq) at 0° C. The reaction temperature is allowed to reach room temperature and then the reaction is stirred for 2 h. On completion of the reaction, it is quenched with saturated aqueous NaHCO₃ solution (40 mL) and extracted with ethyl acetate (2×55 mL). The organic layer is dried over Na₂SO₄, filtered, and evaporated under reduced pressure and the resulting solid is triturated with n-pentane to afford the title compound (2.8 g; 73%) as light brown solid. Mass m/z 233 [M+H]⁺; ¹H-NMR (400 MHz, DMSO-d₆): 10.28 (s, 1H), 8.61 (s, 1H).

### Step 5: (2,4-dichlorothieno[3,2-d]pyrimidin-6-yl)methanol

To a solution of 2,4-dichlorothieno[3,2-d]pyrimidine-6-carbaldehyde (1 g, 4.32 mmol, 1 eq) in dry THE (15 ml) is added NaBH₄ (329 mg, 8.65 mmol, 2 eq) at 0° C. The reaction mixture is stirred at the same temperature for 2 h. The progress of the reaction is monitored by TLC. After completion, the reaction mixture is quenched with water (20 mL) and extracted with EtOAc (2×35 mL). The combined organic layer is dried over Na₂SO₄, filtered, and evaporated under reduced pressure to obtain a crude product which is triturated with n-pentane to afford the title compound (650 mg; 65%) as a light brown solid. Mass m/z 234.9 [M+H]⁺; ¹H-NMR (400 MHz, DMSO-d₆): 7.55 (t, J=1.6, 1H), 6.23 (t, J=5.6 Hz, 1H), 4.92 (dd, J=1.2 Hz, J=5.6 Hz, 2H).

### Step 6: (2-chloro-4-(1,4-oxazepan-4-yl)thieno[3,2-d]pyrimidin-6-yl)methanol

To a stirred solution of (2,4-dichlorothieno[3,2-d]pyrimidin-6-yl)methanol (100 mg, 0.42 mmol, 1 eq.) in IPA (4 mL) is added 1,4-oxazepane (64 mg, 0.64 mmol, 1.5 eq) and DIPEA (0.15 mL, 0.85 mmol, 2 eq) at room temperature. The reaction mixture is heated to 80° C. and maintain for 12 h. The progress of the reaction is monitored by TLC. After completion of the reaction, it is diluted with EtOAc (2×30 mL) and washed with water (10 mL). Finally, the organic layer is dried over anhydrous Na₂SO₄, filtered, and evaporated under reduced pressure to obtain a solid which is triturated with diethyl ether to afford the title compound (85 mg; 67%) as an off-white solid. Mass m/z 300.1 [M+H]+; ¹H-NMR (400 MHz, DMSO-d₆): 7.2 (t, J=1.2 Hz, 1H), 5.953 (t, J=5.6 Hz, 1H), 4.81 (dd, J=0.8 Hz, J=5.6 Hz, 2H), 4.03-4.00 (m, 4H), 3.82 (t, J=5.0 Hz, 2H), 3.67 (t, J=5.6 Hz, 2H), 1.97-1.94 (m, 2H).

### Step 7: (4-(1,4-oxazepan-4-yl)-2-(3-(m-tolyl)-1H-pyrazol-1-yl)thieno[3,2-d]pyrimidin-6-yl)methanol

To a solution of (2-chloro-4-(1,4-oxazepan-4-yl)thieno[3,2-d]pyrimidin-6-yl)methanol (85 mg 0.28 mmol, 1 eq) in a (1:1) mixture of 1,4-dioxane:toluene (3 mL), is added 3-(m-tolyl)-1H-pyrazole (45 mg, 0.28 mmol, 1 eq), and K₃PO₄ (120 mg, 0.56 mmol, 2 eq). The reaction mixture is degassed for 10 min under nitrogen followed by addition of Pd₂(dba)₃ (26 mg, 0.028 mmol, 0.1 eq) and t-Bu-XPhos (24 mg, 0.056 mmol, 0.2 eq) at room temperature. The reaction temperature is raised to 120° C. and stirred for 6 h. The progress of the reaction is monitored by TLC and after completion, it is diluted with EtOAc (2×40 mL) and washed with water (15 mL). Finally, the organic layer is dried over anhydrous Na₂SO₄, filtered, and evaporated under reduced pressure to provide a brownish gummy material which is purified by RP Flash column chromatography (C18-6 g RP column) eluted with 50-55% acetonitrile/water to afford the title compound (25 mg; 21%) as off-white solid. Mass m/z 422.2 [M+H]⁺; ¹H-NMR (400 MHz, DMSO-d₆): 8.68 (d, J=2.8, 1H), 7.79 (br s, 1H), 7.75 (d, J=7.6, 1H), 7.35 (t, J=7.2 Hz, 2H), 7.20 (d, J=7.6, 1H), 7.01 (d, J=2.4, 1H), 5.92 (br s, 1H), 4.83 (br s, 1H), 4.15-4.10 (m, 4H), 3.87 (t, J=5.6 Hz, 2H), 3.69 (t, J=5.2 Hz, 2H), 2.39 (s, 3H) 2.08-2.01 (m, 2H).

### Step 8: 4-(6-(chloromethyl)-2-(3-(m-tolyl)-1H-pyrazol-1-yl)thieno[3,2-d]pyrimidin-4-yl)-1,4-oxazepane

To a stirred solution of (4-(1,4-oxazepan-4-yl)-2-(3-(m-tolyl)-1H-pyrazol-1-yl) thieno[3,2-d]pyrimidin-6-yl)methanol (25 mg, 0.06 mmol, 1 eq.), in DCM (5 mL) at 0° C. is added SOCl₂ (0.05 mL). The reaction is warmed to room temperature and stirred for 2 h. The progress of the reaction was monitored by TLC and after completion, it is evaporated under reduced pressure to provide the title compound (25 mg; 96%) as light-yellow solid. Mass m/z 440.1 [M+H]⁺; ¹H-NMR (400 MHz, DMSO-d₆): 8.72 (d, J=2.4, 1H), 7.80 (br s, 1H), 7.76 (d, J=7.6, 1H), 7.61 (s, 1H), 7.36 (t, J=7.2 Hz, 2H), 7.21 (d, J=7.6, 1H), 7.06 (d, J=2.4, 1H), 5.21 (s, 2H), 4.17-4.11 (m, 4H), 3.88 (t, J=5.6 Hz, 2H), 3.71 (t, J=5.2 Hz, 2H), 2.39 (s, 3H) 2.06-2.00 (m, 2H).

### Step 9: 4-(6-((4-(methylsulfonyl)piperazin-1-yl)methyl)-2-(3-(m-tolyl)-1H-pyrazol-1-yl)thieno[3,2-d]pyrimidin-4-yl)-1,4-oxazepane

To a stirred solution of 4-(6-(chloromethyl)-2-(3-(m-tolyl)-1H-pyrazol-1-yl)thieno[3,2-d]pyrimidin-4-yl)-1,4-oxazepane (25 mg, 0.05 mmol, 1 eq.) in IPA (3 mL) is added 1-(methylsulfonyl)piperazine (14 mg, 0.085 mmol, 1.5 eq) and DIPEA (0.02 mL, 0.11 mmol, 2 eq) at room temperature. The reaction mixture is heated to 80° C. and maintained for 12 h. The progress of the reaction is monitored by TLC. After completion of the reaction, it is diluted with EtOAc (2×20 mL) and washed with water (10 mL). Finally, the organic layer is dried over anhydrous Na₂SO₄, filtered, and evaporated under reduced pressure to provide a solid which is purified by RP Flash column chromatography (C18-6 g RP column) using water/acetonitrile eluting with 50-55% acetonitrile/water to afford the title compound (10 mg; 31%) as an off-white solid. Mass m/z 568.2 [M+H]⁺; ¹H-NMR (400 MHz, DMSO-d₆): 8.68 (d, J=2.4, 1H), 7.78 (br s, 1H), 7.75 (d, J=7.6, 1H), 7.42 (s, 1H), 7.35 (t, J=7.2 Hz, 2H), 7.20 (d, J=7.6, 1H), 7.02 (d, J=2.4, 1H), 4.15-4.09 (m, 4H), 3.91 (s, 2H), 3.87 (t, J=5.6 Hz, 2H), 3.70 (t, J=5.2 Hz, 2H), 3.17 (t, J=4.4 Hz, 4H), 2.90 (s, 3H), 2.61 (Br s, 4H), 2.39 (s, 3H) 2.05-1.99 (m, 2H).

### Example 72

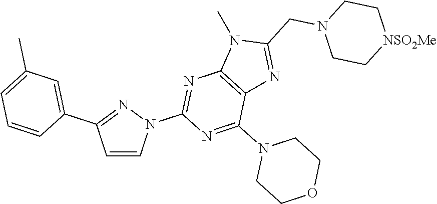

### Step 8: 4-(2-(chloromethyl)-5-(3-(m-tolyl)-1H-pyrazol-1-yl)thieno[3,2-b]pyridin-7-yl)morpholine

Following step 7 of the procedure for Example 75, to a stirred solution of (3-methyl-7-morpholino-5-(3-(m-tolyl)-1H-pyrazol-1-yl)-3H-imidazo[4,5-b]pyridin-2-yl)methanol (350 mg, 0.86 mmol, 1.0 equiv.) in THE (10 mL) is added SOCl₂ (0.2 mL, 2.59 mmol, 3.0 equiv.) at 0° C. The reaction solution is heated to 65° C. and stirred for 3 h. The reaction mixture is concentrated under reduced pressure and the resulting solid is triturated with diethyl ether to afford the title compound (300 mg, 83%) as an off white solid. Mass [m/z] 423 [M+H]; #¹H-NMR (400 MHz, DMSO-d₆): δ 8.70 (d, J=2.8 Hz, 1H), 7.76-7.79 (m, 2H), 7.35 (t, J=7.6 Hz, 1H), 7.24 (s, 1H), 7.19 (d, J=8.4 Hz, 1H), 7.04 (d, J=2.8 Hz, 1H), 5.08 (s, 2H), 3.96-3.99 (m, 4H), 3.85 (s, 3H), 3.81-3.83 (m, 4H), 2.39 (s, 3H). #

### Step 9: 4-(3-methyl-2-((4-(methylsulfonyl)piperazin-1-yl)methyl)-5-(3-(m-tolyl)-1H-pyrazol-1-yl)-3H-imidazo[4,5-b]pyridin-7-yl)morpholine

To a stirred solution of 4-(2-(chloromethyl)-5-(3-(m-tolyl)-1H-pyrazol-1-yl)thieno[3,2-b]pyridin-7-yl)morpholine (50 mg, 1.0 eq.) in THE (10 vol) at RT is added 1-(methylsulfonyl)piperazine (2 eq.) followed by K₂CO₃ (2.5 equiv.). The reaction mixture is stirred at 70° C. for 16 h and then it is diluted with water (10 vol) and extracted with EtOAc (2×10 vol). The combined organic layers are dried over anhydrous Na₂SO₄, filtered and evaporated under reduced pressure to obtain solid is purified by silica gel column chromatography (60-120 mesh) eluting with 2-5% MeOH/DCM, to afford the title compound (15 mg; 25%) as an off white solid. Mass [m/z] 551 [M+H]⁺; ¹H-NMR (400 MHz, DMSO-d₆): δ 8.68 (d, J=2.8 Hz, 1H), 7.76-7.79 (m, 2H), 7.35 (t, J=7.6 Hz, 1H), 7.18-7.21 (m, 2H), 7.02 (d, J=2.4 Hz, 1H), 3.94-3.96 (m, 4H), 3.81-3.84 (m, 9H), 3.12 (br s, 4H), 2.86 (s, 3H), 2.56 (br s, 4H), 2.39 (s, 3H).

### Example 73

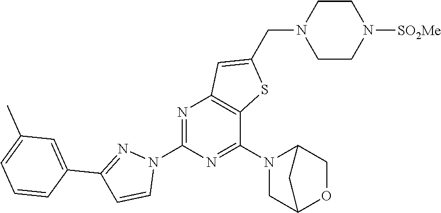 [SMILES: Cc1cccc(-c2ccn(-c3nc(N4CC5CC4CO5)c4sc(CN5CCN(S(C)(=O)=O)CC5)cc4n3)n2)c1]

### Step 6: (2-chloro-4-(3,6-dihydro-2H-pyran-4-yl)thieno[3,2-d]pyrimidin-6-yl)methanol

Following Step 5 of Example 71, to a stirred solution of (2,4-dichlorothieno[3,2-d]pyrimidin-6-yl)methanol (100 mg, 0.42 mmol, 1 eq.) in IPA (4 mL) is added 2-oxa-5-azabicyclo[2.2.1]heptane (63 mg, 0.64 mmol, 1.5 eq) and DIPEA (0.15 mL, 0.85 mmol, 2 eq) at room temperature. The reaction mixture is heated to 80° C. and maintained for 12 h. The progress of the reaction is monitored by TLC. After completion of the reaction, it is diluted with EtOAc (2×30 mL) and washed with water (10 mL). Finally, the organic layer is dried over anhydrous Na₂SO₄, filtered and evaporated under reduced pressure to provide a crude product which is triturated with diethyl ether to afford the title compound (100 mg; 78%) as an off-white solid. Mass m/z 298 [M+H]⁺; ¹H-NMR (400 MHz, DMSO-d₆): 7.18 (s, 1H), 5.93 (t, J=6.0 Hz, 1H), 5.16 (s, 1H), 4.80 (dd, J=1.2 Hz, J=5.6 Hz, 2H), 4.76 (s, 1H), 3.86-3.73 (m, 4H), 2.0-1.94 (m, 2H).

### Step 7: 5-(2-chloro-6-(chloromethyl)thieno[3,2-d]pyrimidin-4-yl)-2-oxa-5-azabicyclo[2.2.1]heptane

To a stirred solution of (4-(2-oxa-5-azabicyclo[2.2.1]heptan-5-yl)-2-chlorothieno[3,2-d]pyrimidin-6-yl)methanol (100 mg, 0.29 mmol, 1 eq.), in DCM (5 mL) at 0° C. is added SOCl₂ (0.1 mL). The reaction is allowed to warm to room temperature and then it is stirred for 2 h. The progress of the reaction is monitored by TLC and after completion, it is evaporated under reduced pressure to afford the title compound (90 mg; 85%) as an off-white solid. Mass m/z 316 [M+H]⁺; ¹H-NMR (400 MHz, DMSO-d₆): 7.44 (s, 1H), 5.16 (s, 3H), 4.76 (s, 1H), 3.94-3.74 (m, 5H), 2.0-1.94 (m, 2H).

### Step 8: 5-(2-chloro-6-((4-(methylsulfonyl)piperazin-1-yl)methyl)thieno[3,2-d]pyrimidin-4-yl)-2-oxa-5-azabicyclo[2.2.1]heptane

To a stirred solution of 5-(2-chloro-6-(chloromethyl)thieno[3,2-d]pyrimidin-4-yl)-2-oxa-5-azabicyclo[2.2.1]heptane (50 mg, 0.15 mmol, 1 eq.) in IPA (3 mL) is added 1-(methylsulfonyl)piperazine (39 mg, 0.23 mmol, 1.5 eq.) and DIPEA (0.06 mL, 0.39 mmol, 2 eq) at room temperature. The reaction is heated to 80° C. and maintained for 12 h. The progress of the reaction is monitored by TLC. After completion of the reaction, it is diluted with EtOAc (2×25 mL) and washed with water (10 mL). Finally, the organic layer is dried over anhydrous Na₂SO₄, filtered and evaporated under reduced pressure to provide a crude product which is purified by silica gel column chromatography (60-120 mesh) eluting with 40-50% EtOAc in hexane to afford the title compound (40 mg; 57%) as an off-white solid. Mass m/z 444.1 [M+H]⁺; ¹H-NMR (400 MHz, DMSO-d₆): 7.16 (s, 1H), 5.90 (br s, 1H), 4.77 (s, 1H), 3.96-3.93 (m, 3H), 3.89 (t, J=9.6 Hz, 1H), 3.84 (s, 2H), 3.28 (t, J=4.8 Hz, 4H), 2.80 (s, 3H), 2.65 (t, J=4.8 Hz, 4H), 2.05 (d, J=10.0 Hz, 1H), 1.98 (d, J=10.0 Hz, 1H).

### Step 9: 5-(6-((4-(methylsulfonyl)piperazin-1-yl)methyl)-2-(3-(m-tolyl)-1H-pyrazol-1-yl)thieno[3,2-d]pyrimidin-4-yl)-2-oxa-5-azabicyclo[2.2.1]heptane

To a solution of 5-(2-chloro-6-((4-(methylsulfonyl)piperazin-1-yl)methyl)thieno[3,2-d]pyrimidin-4-yl)-2-oxa-5-azabicyclo[2.2.1]heptane (40 mg 0.09 mmol, 1 eq) in a (1:1) mixture of 1,4-dioxane: toluene (3 mL) is added 3-(m-tolyl)-1H-pyrazole (14 mg, 0.09 mmol, 1 eq), and K₃PO₄ (38 mg, 0.18 mmol, 2 eq). The reaction mixture is degassed for 10 min under nitrogen, followed by the addition of Pd₂(dba)₃ (8 mg, 0.009 mmol, 0.1 eq) and t-Bu-XPhos (8 mg, 0.018 mmol, 0.2 eq) at room temperature. The reaction temperature is raised to 120° C. and the reaction is stirred for 6 h. The progress of the reaction is monitored by TLC and after completion, it is diluted with EtOAc (25 mL) and washed with water (10 mL). Finally, the organic layer is dried over anhydrous Na₂SO₄, filtered, and evaporated under reduced pressure to provide a brownish gummy material which is purified by RP Flash column chromatography (C18-6 g RP column) eluting with 60-65% acetonitrile/water to afford the title compound (14 mg; 27%) as an off-white solid. Mass m/z 566.2 [M+H]⁺; ¹H-NMR (400 MHz, DMSO-d₆): 8.73 (d, J=2.4 Hz, 1H), 7.79 (Br s, 1H), 7.74 (d, J=8.0 Hz, 1H), 7.40 (s, 1H), 7.35 (t, J=7.6 Hz, 1H), 7.19 (d, J=7.6 Hz, 1H), 7.01 (d, J=2.8 Hz, 1H), 5.41 (Br s, 1H), 4.80 (s, 1H), 3.98-3.83 (m, 6H), 3.15 (d, J=4.4 Hz, 4H), 2.90 (s, 3H), 2.60 (Br s, 4H), 2.39 (s, 3H), 2.05 (d, J=9.6 Hz, 1H), 1.98 (s, 1H).

### Example 74

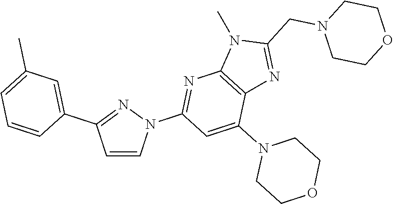 [SMILES: Cc1cccc(-c2ccn(-c3cc(N4CCOCC4)c4nc(CN5CCOCC5)n(C)c4n3)n2)c1]

### Step 9: 4-(3-methyl-2-(morpholinomethyl)-5-(3-(m-tolyl)-1H-pyrazol-1-yl)-3H-imidazo[4,5-b]pyridin-7-yl)morpholine

According to the procedure of Example 72, 4-(2-(chloromethyl)-5-(3-(m-tolyl)-1H-pyrazol-1-yl)thieno[3,2-b]pyridin-7-yl)morpholine (50 mg) is reacted with morpholine (2 eq.) to provide a solid which is purified by silica gel column chromatography (60-120 mesh) eluting with 15-20% ethyl acetate/hexane, to afford the title compound (20 mg; 40%) as an off-white solid. Mass [m/z] 474 [M+H]⁺; ¹H-NMR (400 MHz, CDCl₃): δ 8.62 (d, J=2.8 Hz, 1H), 7.78 (s, 1H), 7.73 (d, J=7.6 Hz, 1H), 7.31-7.34 (m, 2H), 7.16 (d, J=7.6 Hz, 1H), 6.75 (d, J=2.4 Hz, 1H), 4.00-4.02 (m, 4H), 3.93-3.95 (m, 4H), 3.89 (s, 3H), 3.75 (s, 2H), 3.71 (t, J=4.4 Hz, 4H), 2.54 (t, J=4.4 Hz, 4H), 2.43 (s, 3H).

### Example 75

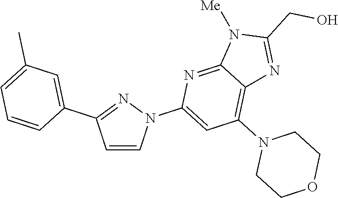 [SMILES: Cc1cccc(-c2ccn(-c3cc(N4CCOCC4)c4nc(CO)n(C)c4n3)n2)c1]

### Step 1: 4,6-dichloro-3-nitropyridin-2-amine

To conc. H₂SO₄ (25 mL, 10 vol.) is added 4,6-dichloropyridin-2-amine (5 g, 30.86 mmol, 1.0 eq) portion-wise at −5° C., and the reaction is allowed to reach room temperature, then it is stirred for 30 min. The reaction is re-cooled to −5° C., and then concentrated nitric acid (2 ml) is added dropwise. The reaction mixture is allowed to warm to room temperature and it is stirred for an additional 1 h. After completion, the mixture is slowly poured into crushed ice, followed by adjustment of the pH to ˜8 using a saturated NaHCO₃ solution. The resulting solid is filtered and washed with water and dried. The solid is further purified by column chromatography (60-120 mesh silica gel) eluting with 2-5% ethyl acetate in hexane to afford the title compound (3.0 g, 47%) as yellow solid. Mass m/z=206 [M−H]⁺; ¹H-NMR (400 MHz, CDCl₃): δ 6.83 (s, 1H), 6.28 (br s, 2H).

### Step 2: 4,6-dichloropyridine-2,3-diamine

To a stirred solution of 4,6-dichloro-3-nitropyridin-2-amine (4 g, 19.41 mmol, 1.0 equiv.) in IPA (40 mL) is added iron powder (3.2 g, 58.2 mmol, 3.0 equiv.) and the mixture is stirred for 5 min. To this mixture is added 6 N HCl (16 mL, 4 vol.) dropwise at 0° C. The reaction mixture is allowed to reach room temperature and is then stirred for 4 h. After completion of the reaction (monitored by TLC), saturated aqueous NaHCO₃ solution is added until the pH is about 8. The reaction mixture is filtered through Celite and the filtrate is extracted with ethyl acetate (2×50 mL). The combined organic layer is washed with brine (25 mL), dried over Na₂SO₄, filtered, and evaporated under reduced pressure to provide a crude compound which is slurried in diethyl ether, filtered, and dried to afford the title compound (2.0 g, 58%) as a pale brown solid. Mass [m/z] 178 [M+H]⁺; ¹H-NMR (400 MHz, CDCl₃): δ 6.77 (s, 1H), 4.48 (br s, 2H), 3.59 (br s, 2H).

### Step 3: 2-((benzyloxy)methyl)-5,7-dichloro-3H-imidazo[4,5-b]pyridine

A stirred mixture of 4,6-dichloropyridine-2,3-diamine (4 g, 22.4 mmol, 1.0 equiv.) and 2-(benzyloxy)acetic acid (7.4 g, 44.9 mmol, 2.0 equiv.) is heated at 150° C. for 16 h. After consumption of the diamine (monitored by TLC), the reaction is diluted with EtOAc (100 mL) and washed with saturated aqueous NaHCO₃ (50 ml) and then with brine (50 mL). The organics are dried over Na₂SO₄, filtered, and concentrated under reduced pressure to provide a solid. The crude solid is purified by silica gel (60-120 mesh) column chromatography using 10-15% of EtOAc/hexane to afford the title compound (3.0 g, 43%) as an off white solid. Mass [m/z] 308 [M+H]⁺; ¹H-NMR (400 MHz, CDCl₃): δ 10.28 (s, 1H), 7.32-7.38 (m, 5H), 7.31 (s, 1H), 4.86 (s, 2H), 4.67 (s, 2H).

### Step 4: 4-(2-((benzyloxy)methyl)-5-chloro-3H-imidazo[4,5-b]pyridin-7-yl)morpholine

To a stirred solution of 2-((benzyloxy)methyl)-5,7-dichloro-3H-imidazo[4,5-b]pyridine (1.0 g, 3.24 mmol, 1.0 equiv.) in ethylene glycol (5 mL) at RT is added morpholine (0.7 mL, 8.11 mmol, 2.5 equiv.) followed by triethylamine (0.9 mL, 6.49 mmol, 2.0 equiv.). The mixture is heated to 120° C. and stirred for 16 h. The progress of the reaction is monitored by TLC. After completion, the reaction is diluted with water (30 mL) to obtain a solid, which is filtered, washed with water and dried to obtained a crude product. The solid is washed with diethyl ether to afford the title compound (0.8 g, 69%) as white solid. Mass [m/z] 359 [M+H]⁺; ¹H-NMR (400 MHz, DMSO-d₆): δ 13.09 (br s, 1H), 7.30-7.40 (m, 5H), 6.51 (s, 1H), 4.65 (s, 2H), 4.57 (s, 2H), 3.88 (t, J=4.0 Hz, 4H), 3.75 (t, J=5.2 Hz, 4H).

### Step 5: 4-(2-((benzyloxy)methyl)-5-chloro-3-methyl-3H-imidazo[4,5-b]pyridin-7-yl)morpholine

To a stirred solution of 4-(2-((benzyloxy)methyl)-5-chloro-3H-imidazo[4,5-b]pyridin-7-yl)morpholine (2.0 g, 5.57 mmol, 1.0 equiv.) in THF/DMF (1:1) (20 mL) is added sodium hydride (0.5 g, 13.96 mmol, 2.5 equiv.) portion-wise at 0° C. and stirred for 30 min. Methyl iodide (0.9 mL, 13.92 mmol, 2.5 eq.) is added dropwise at 0° C. and the reaction mixture is allowed to warm to RT and is stirred at RT for 4 h. The reaction is quenched with cold water and the resulting solid is filtered, washed with water and dried. The solid is triturated with n-pentane to afford the title compound (1.7 g, 82%) as an off white solid. Mass [m/z] 373 [M+H]⁺; ¹H-NMR (400 MHz, CDCl3): δ 7.32-7.35 (m, 5H), 6.41 (s, 1H), 4.73 (s, 2H), 4.57 (s, 2H), 3.87-3.92 (m, 8H), 3.80 (s, 3H).

### Step 6: 4-(2-((benzyloxy)methyl)-3-methyl-5-(3-(m-tolyl)-1H-pyrazol-1-yl)-3H-imidazo[4,5-b]pyridin-7-yl)morpholine

To a stirred solution of 4-(2-((benzyloxy)methyl)-5-chloro-3-methyl-3H-imidazo[4,5-b]pyridin-7-yl)morpholine (2.0 g, 5.36 mmol, 1.0 eq.) and 3-(m-tolyl)-1H-pyrazole (1.27 g, 8.04 mmol, 1.5 eq.) in 1,4-dioxane: toluene (1:1) (20 mL) is added K₃PO₄ (2.2 g, 10.7 mmol, 2.0 equiv.), and the reaction mixture is degassed for 15 min. Pd₂(dba)₃ (0.5 g, 0.53 mmol, 0.1 equiv.) is added followed by t-Bu-XPhos (0.45 g, 1.07 mmol, 0.2 equiv.) at RT. The resulting mixture is degassed for 15 min and then it is stirred at 110° C. for 16 h. The reaction mixture is diluted with EtOAc and filtered. The filtrate is evaporated under reduced pressure and the resulting residue is purified by reverse phase flash purification (C18, 60 g column) eluting with 50%-60% acetonitrile/water to afford the title compound (1.8 g, 69%) as a white solid. Mass [m/z] 495 [M+H]⁺; ¹H-NMR (400 MHz, CDCl₃): δ 8.62 (d, J=2.4 Hz, 1H), 7.78 (s, 1H), 7.73 (d, J=7.6 Hz, 1H), 7.31-7.36 (m, 7H), 7.16 (d, J=7.6 Hz, 1H), 6.76 (d, J=2.4 Hz, 1H), 4.77 (s, 2H), 4.59 (s, 2H), 4.02-4.04 (m, 4H), 3.93-3.95 (m, 4H), 3.84 (s, 3H), 2.43 (s, 3H). #

### Step 7: (3-methyl-7-morpholino-5-(3-(m-tolyl)-11H-pyrazol-1-yl)-3H-imidazo[4,5-b]pyridin-2-yl)methanol:#

To a stirred solution of 4-(2-((benzyloxy)methyl)-3-methyl-5-(3-(m-tolyl)-1H-pyrazol-1-yl)-3H-imidazo[4,5-b]pyridin-7-yl)morpholine (2.3 g, 4.65 mmol, 1.0 eq) in THE (20 mL) is added 10% Pd/C (1.2 g) and the reaction mixture is stirred at RT for 48 h under an H₂ atmosphere at 30 psi. The reaction mixture is filtered through Celite and washed with THF. The filtrate is evaporated under reduced pressure, and the crude product is triturated with diethyl ether to afford the title compound (1.2 g, 64%) as an off white solid. Mass [m/z] 405 [M+H]⁺; #¹H-NMR (400 MHz, CDCl₃): δ 8.58 (d, J=2.8 Hz, 1H), 7.76 (s, 1H), 7.72 (d, J=7.6 Hz, 1H), 7.31-7.35 (m, 2H), 7.17 (d, J=7.2 Hz, 1H), 6.72 (d, J=6.8 Hz, 1H), 4.85 (d, J=5.2 Hz, 2H), 3.93-3.99 (m, 8H), 3.81 (s, 3H), 2.92 (t, J=5.6 Hz, 1H), 2.44 (s, 3H). #

### Example 76

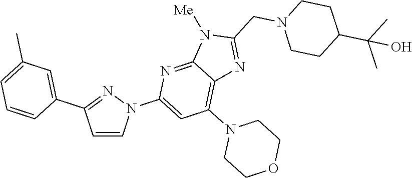 [SMILES: Cc1cccc(-c2ccn(-c3cc(N4CCOCC4)c4nc(CN5CCC(C(C)(C)O)CC5)n(C)c4n3)n2)c1]

### Step 9: 2-(1-((3-methyl-7-morpholino-5-(3-(m-tolyl)-1H-pyrazol-1-yl)-3H-imidazo[4,5-b]pyridin-2-yl)methyl)piperidin-4-yl)propan-2-ol

According to the procedure of Example 72, 4-(2-(chloromethyl)-5-(3-(m-tolyl)-1H-pyrazol-1-yl)thieno[3,2-b]pyridin-7-yl)morpholine (50 mg) is reacted with 2-(piperidin-4-yl)propan-2-ol (2 eq.) to provide a solid which is purified by silica gel column chromatography (60-120 mesh) eluting with 2-5% MeOH/DCM, to afford the title compound (12 mg; 20%) as off white solid. Mass [m/z] 530 [M+H]⁺; ¹H-NMR (400 MHz, DMSO-d₆): δ 8.68 (d, J=2.4 Hz, 1H), 7.76-7.79 (m, 2H), 7.35 (t, J=7.6 Hz, 1H), 7.18-7.21 (m, 2H), 7.02 (d, J=2.4 Hz, 1H), 4.05 (s, 1H), 3.94-3.95 (m, 4H), 3.83 (s, 8H), 3.71 (s, 2H), 2.88 (d, J=10.8 Hz, 2H), 2.40 (s, 3H), 1.98 (t, J=10.8 Hz, 2H), 1.66 (d, J=10.4 Hz, 2H), 1.18-1.26 (m, 2H), 1.02 (s, 6H).

### Example 77

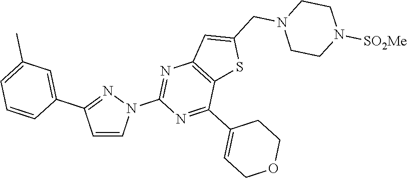 [SMILES: Cc1cccc(-c2ccn(-c3nc(C4=CCOCC4)c4sc(CN5CCN(S(C)(=O)=O)CC5)cc4n3)n2)c1]

### Step 6: (4-(2-oxa-5-azabicyclo[2.2.1]heptan-5-yl)-2-chlorothieno[3,2-d]pyrimidin-6-yl)methanol (Compound-73-1a)

Following Step 5 of Example 71, to a stirred solution of (2,4-dichlorothieno[3,2-d]pyrimidin-6-yl)methanol (150 mg, 0.64 mmol, 1 eq.) in 1,4-dioxane: water (9:1) (3 mL) is added (3,6-dihydro-2H-pyran-4-yl)boronic acid (74 mg, 0.576 mmol, 0.9 eq), and Pd(dppf)Cl₂-DCM (47 mg, 0.064 mmol, 0.1 eq) followed by K₂CO₃ (178 mg, 1.28 mmol, 2 eq). The reaction mixture is degassed for 20 min under nitrogen at room temperature and then is stirred for 12 h. The progress of the reaction is monitored by TLC. After completion of the reaction, it is diluted with EtOAc (2×40 mL) and washed with water (20 mL). Finally, the organic layer is dried over anhydrous Na₂SO₄, filtered, and evaporated under reduced pressure to provide a crude product which is purified by silica gel (60-120 mesh) column chromatography eluting with 30-35% EtOAc in hexane to afford the title compound (85 mg; 47%) as an off-white solid. Mass m/z 283 [M+H]⁺; ¹H-NMR (400 MHz, DMSO-d₆): 7.45 (s, 1H), 7.08 (t, J=12.0, 1H), 6.10 (t, J=5.6 Hz, 1H), 4.90 (dd, J=12.0, 5.6 Hz, 2H), 4.39 (q, J=2.8, 5.6 Hz, 2H), 3.86 (t, J=5.6 Hz, 2H), 2.67-2.66 (m, 2H).

### Step 7: 2-chloro-6-(chloromethyl)-4-(3,6-dihydro-2H-pyran-4-yl)thieno[3,2-d]pyrimidine

To a stirred solution of (2-chloro-4-(3,6-dihydro-2H-pyran-4-yl)thieno[3,2-d]pyrimidin-6-yl)methanol (85 mg, 0.30 mmol, 1 eq.), in DCM (5 mL) at 0° C. is added SOCl₂ (0.1 mL). The reaction is allowed to warm to room temperature and then it is stirred for 2 h. The progress of the reaction is monitored by TLC and after completion, it is evaporated under reduced pressure to provide a solid which is triturated with n-pentane to afford the title compound (55 mg; 61%) as a yellow solid. Mass m/z 301 [M+H]⁺; ¹H-NMR (400 MHz, DMSO-d₆: 7.72 (d, J=4.4, 1H), 7.09-7.08 (m, 1H), 5.27 (t, J=7.6 Hz, 2H), 4.41-4.38 (m, 2H), 3.87 (t, J=5.6 Hz, 2H), 2.67 (d, J=2.0 Hz, 2H).

### Step 8: 2-chloro-4-(3,6-dihydro-2H-pyran-4-yl)-6-((4-(methylsulfonyl)piperazin-1-yl)methyl)thieno[3,2-d]pyrimidine

To a stirred solution of 2-chloro-6-(chloromethyl)-4-(3,6-dihydro-2H-pyran-4-yl)thieno[3,2-d]pyrimidine (55 mg, 0.18 mmol, 1 eq.) in IPA (3 mL) is added 1-(methylsulfonyl)piperazine (45 mg, 0.27 mmol, 1.5 eq) and DIPEA (0.07 mL, 0.36 mmol, 2 eq) at room temperature. The reaction mixture is heated to 80° C. and it is continued stirring for 12 h. The progress of the reaction is monitored by TLC. After completion of the reaction, it is diluted with EtOAc (2×30 mL) and washed with water (15 mL). Finally, the organic layer is dried over anhydrous Na₂SO₄, filtered, and evaporated under reduced pressure to provide a crude solid which is purified by silica gel column (60-120 mesh) chromatography eluting with 40-50% EtOAc in hexane to afford the title compound (45 mg; 57%) as an off-white solid. Mass m/z 429.1 [M+H]⁺.

### Step 9: 4-(3,6-dihydro-2H-pyran-4-yl)-6-((4-(methylsulfonyl)piperazin-1-yl)methyl)-2-(3-(m-tolyl)-1H-pyrazol-1-yl)thieno[3,2-d]pyrimidine

To a solution of 2-chloro-4-(3,6-dihydro-2H-pyran-4-yl)-6-((4-(methylsulfonyl)piperazin-1-yl)methyl)thieno[3,2-d]pyrimidine (40 mg 0.09 mmol, 1 eq) in a (1:1) mixture of 1,4-dioxane:toluene (3 mL) is added 3-(m-tolyl)-1H-pyrazole (15 mg, 0.09 mmol, 1 eq), and K₃PO₄ (39 mg, 0.18 mmol, 2 eq). The reaction mixture is degassed for 10 min under nitrogen followed by the addition of Pd₂(dba)₃ (8 mg, 0.009 mmol, 0.1 eq) and t-butyl-XPhos (8 mg, 0.018 mmol, 0.2 eq) at room temperature. The reaction temperature is raised to 120° C. and it is stirred for 6 h. The progress of the reaction is monitored by TLC and after completion, it is diluted with EtOAc (2×30 mL) and washed with water (10 mL). Finally, the organic layer is dried over anhydrous Na₂SO₄, filtered, and evaporated under reduced pressure to provide a brownish gummy material which is purified by Prep HPLC (Method-B: X-Bridge, C18, 250×19.1 mm, 5 μm; Mobile phase: [ACN: 0.1% of FA in Water]; time/B %: 0/45, 15/70, 25/90) to afford the title compound (3 mg; 6%) as off-white solid. Mass m/z 551.2 [M+H]⁺; ¹H-NMR (400 MHz, DMSO-d₆): 8.82 (d, J=2.4, 1H), 7.82 (s, 1H), 7.77 (d, J=8.0 Hz, 1H), 7.70 (s. 1H), 7.37 (t, J=7.6 Hz, 1H), 7.21 (d, J=8.4 Hz, 1H), 7.15 (s, 1H), 7.10 (d, J=2.8 Hz, 1H), 4.43 (d, J=2.8 Hz, 2H), 4.16 (br s, 2H), 3.93 (t, J=5.2 Hz, 2H), 3.21 (br s, 4H), 2.93 (s, 3H), 2.83-2.75 (m, 6H), 2.40 (s, 3H).

### Example 78

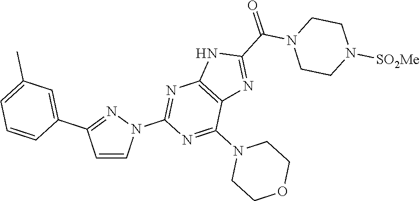 [SMILES: Cc1cccc(-c2ccn(-c3nc(N4CCOCC4)c4nc(C(=O)N5CCN(S(C)(=O)=O)CC5)[nH]c4n3)n2)c1]

### Step 1: (6-morpholino-9-(tetrahydro-2H-pyran-2-yl)-2-(3-(m-tolyl)-1H-pyrazol-1-yl)-9H-purin-8-yl)methanol

To a stirred solution of (2-chloro-6-morpholino-9-(tetrahydro-2H-pyran-2-yl)-9H-purin-8-yl)methanol (0.450 g, 1.27 mmol, 1 eq.), and 3-(m-tolyl)-1H-pyrazole (0.201 g, 1.27 mmol, 1 eq.) dissolved in a mixture of 1,4-dioxane: toluene (1:1) (10 mL) is added Pd₂(dba)₃ (0.116 g, 0.12 mmol, 0.1 eq.), and t-Bu-XPhos (0.108 g, 0.25 mmol, 0.2 eq.), followed by K₃PO₄ (0.54 g, 2.54 mmol, 2 eq.) and the reaction mixture is degassed for 30 min. The reaction mixture is stirred at 110° C. for 16 h. The progress of the reaction is monitored by TLC. After complete consumption of the starting material, the catalyst impurities are filtered by passing the reaction mixture through a Celite pad. The filtrate is diluted with EtOAc (20 mL), washed with water (10 mL), and the organic layer is dried over anhydrous Na₂SO₄, filtered, and evaporated under reduced pressure to obtain a brown liquid. The crude compound is purified by Flash (C18 reverse phase 12 g column) eluting with 70-80% acetonitrile/water to afford the title compound (180 mg; 28%) as an off white solid. Mass m/z 476.2 [M+H]⁺; ¹H-NMR (400 MHz, DMSO-d₆): 8.70 (d, J=2.8 Hz, 1H), 7.78-7.75 (m, 2H), 7.36 (t, J=7.6 Hz 1H) 7.02 (d, J 2.4 Hz, 1H), 7.02 (d, J=2.8 Hz, 1H), 5.81 (dd, J=9.2, 11.20 Hz, 1H), 5.66 (t, J=6.0 Hz, 1H), 4.79-4.67 (m, 2H), 4.31 (br s, 4H), 4.09 (d, J=10.8 Hz, 1H), 3.79-3.69 (m, 5H), 2.84-2.71 (m, 1H), 2.39 (s, 3H), 2.04-2.01 (m, 1H), 1.84 (d, J=11.6 Hz, 1H), 1.75-1.59 (m, 3H).

### Step 2: (6-morpholino-2-(3-(m-tolyl)-1H-pyrazol-1-yl)-9H-purin-8-yl)methanol

To a stirred solution of (6-morpholino-9-(tetrahydro-2H-pyran-2-yl)-2-(3-(m-tolyl)-1H-pyrazol-1-yl)-9H-purin-8-yl)methanol (80 mg, 0.16 mmol, 1 eq.) in 1,4-dioxane (2 mL), is added 4M HCl in 1,4-dioxane (2 mL) at 0° C. The reaction mixture is stirred at 0° C. for 2 h. The progress of the reaction is monitored by TLC, and after completion of the reaction, it is concentrated under reduced pressure to obtain a sticky solid which is triturated with n-pentane, filtered, and dried under vacuum to afford the title compound (60 mg; 92%) as an off white solid. Mass m/z 392.2 [M+H]⁺; ¹H-NMR (400 MHz, DMSO-d₆): 8.68 (d, J=2.4 Hz, 1H), 7.77 (s, 1H), 7.73 (d, J=8.0 Hz, 1H), 7.35 (t, J=7.6 Hz, 1H), 7.19 (d, J=7.6 Hz, 1H), 7.01 (d, J=2.4 Hz, 1H), 4.61 (br s, 2H), 4.28 (br s, 4H), 3.77 (t, J=4.4 Hz, 4H), 2.39 (s, 3H).

### Step 3: 6-morpholino-2-(3-(m-tolyl)-1H-pyrazol-1-yl)-9H-purine-8-carboxylic acid

To a stirred solution of (6-morpholino-2-(3-(m-tolyl)-1H-pyrazol-1-yl)-9H-purin-8-yl) methanol (100 mg, 0.25 mmol, 1 eq.) in water (1 mL) at room temperature, is added 1N NaOH (0.5 mL) followed by the addition of KMnO₄ (60 mg, 0.38 mmol, 1.5 eq) at the same temperature. The resulting reaction mixture is stirred at 90° C. for 16 h. The progress of the reaction is monitored by TLC, and after completion of the reaction, the reaction mixture is cooled to room temperature and its pH is adjusted up to 5 using acetic acid. The reaction mass is passed through a Celite pad and the filtrate is extracted with EtOAc (2×20 mL). The organic layer is dried over Na₂SO₄, filtered, and evaporated under reduced pressure to obtain a crude product which is triturated with n-pentane to afford the title compound (55 mg; 53%) as a brown solid. Mass m/z 406 [M+H]⁺; ¹H-NMR (400 MHz, DMSO-d₆): 14.09 (br s, 1H), 11.95 (Br s, 1H), 8.70 (d, J=3.2 Hz, 1H), 7.38-7.36 (m, 2H), 7.3-7.01 (m, 3H), 7.01 (d, J=2.4 Hz, 1H), 4.30 (br s, 4H), 4.28 (br s, 4H), 3.70 (s, 4H), 2.39 (s, 3H).

### Step 4: (4-(methylsulfonyl)piperidin-1-yl)(6-morpholino-2-(3-(m-tolyl)-1H-pyrazol-1-yl)-9H-purin-8-yl)methanone

To a stirred solution of 6-morpholino-2-(3-(m-tolyl)-1H-pyrazol-1-yl)-9H-purine-8-carboxylic acid (30 mg, 0.04 mmol, 1 eq.) in DCM (1 mL), is added 1-(methylsulfonyl)piperazine (13 mg, 0.05 mmol, 1.2 eq), propylphosphonic anhydride (50% in EtOAc) (72 mg, 0.09 mmol, 2 eq), and triethylamine (12 mg, 0.09 mmol, 2 eq) at 0° C., then the reaction mixture is stirred at room temperature for 12 h. The reaction is monitored by TLC. After completion of the reaction, it is diluted with DCM and washed with water. The organic layer is dried over Na₂SO₄, filtered, and evaporated under reduced pressure to obtain a crude product which is purified by Flash RP (C18 reverse phase 6 g column) eluting with 50% acetonitrile/water to afford the title compound (8 mg; 20%) as a white solid. Mass m/z 552.2 [M+H]⁺; ¹H-NMR (400 MHz, DMSO-d₆): 13.86 (br s, 1H), 8.71 (d, J=2.4 Hz, 1H), 7.77 (s, 1H), 7.73 (d, J=7.6 Hz, 1H), 7.35 (t, J=7.6 Hz, 1H), 7.19 (d, J=7.6 Hz, 1H), 7.02-7.0 (m, 1H), 4.37-4.30 (br s, 6H), 3.80-3.79 (m, 6H), 3.26 (br s, 4H), 2.97 (s, 3H), 2.39 (s, 3H).

### Example 79

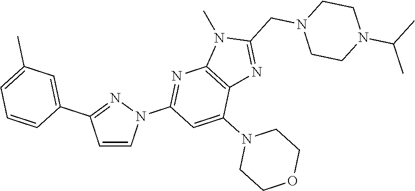 [SMILES: Cc1cccc(-c2ccn(-c3cc(N4CCOCC4)c4nc(CN5CCN(C(C)C)CC5)n(C)c4n3)n2)c1]

### Step 9: 4-(2-((4-isoproylpiperazin-1-yl)methyl)-3-methyl-5-(3-(m-tolyl)-1H-pyrazol-1-yl)-3H-imidazo[4,5-b]pyridin-7-yl)morpholine

According to the procedure of Example 72, 4-(2-(chloromethyl)-5-(3-(m-tolyl)-1H-pyrazol-1-yl)thieno[3,2-b]pyridin-7-yl)morpholine (50 mg) is reacted with 1-isopropylpiperazine (2 eq.) to provide a solid which is purified by silica gel column chromatography (60-120 mesh) eluting with 2-5% MeOH/DCM, to afford the title compound (18 mg; 32%) as an off-white solid. Mass [m/z] 515 [M+H]⁺; ¹H-NMR (400 MHz, MeOH): δ 8.64 (d, J=2.4 Hz, 1H), 7.75 (s, 1H), 7.69 (d, J=8.0 Hz, 1H), 7.32 (d, J=7.6 Hz, 1H), 7.28 (s, 1H), 7.18 (d, J=7.6 Hz, 1H), 6.85 (d, J=2.4 Hz, 1H), 3.90 (d, J=6.8 Hz, 12H), 3.82 (d, J=4.4 Hz, 2H), 2.66-2.76 (m, 8H), 2.41 (s, 3H), 1.14 (d, J=6.4 Hz, 6H).

### Example 82

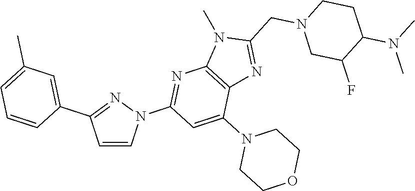 [SMILES: Cc1cccc(-c2ccn(-c3cc(N4CCOCC4)c4nc(CN5CCC(N(C)C)C(F)C5)n(C)c4n3)n2)c1]

### Step 9: 3-fluoro-N,N-dimethyl-1-((3-methyl-7-morpholino-5-(3-(m-tolyl)-1H-pyrazol-1-yl)-3H-imidazo[4,5-b]pyridin-2-yl)methyl)piperidin-4-amine

According to the procedure of Example 72, 4-(2-(chloromethyl)-5-(3-(m-tolyl)-1H-pyrazol-1-yl)thieno[3,2-b]pyridin-7-yl)morpholine (100 mg) is reacted with 3-fluoro-N,N-dimethylpiperidin-4-amine (2 eq.) to provide a solid which is purified by silica gel column chromatography, to afford the title compound (20 mg; 15%) as an off-white solid. Mass [m/z]=533 [M+H]⁺; ¹H-NMR (400 MHz, MeOH): δ 9.67 (s, 1H), 8.68 (d, J=2.4 Hz, 1H), 7.76-7.78 (m, 2H), 7.35 (t, J=3.6 Hz, 1H), 7.22 (s, 1H), 7.18 (d, J=7.6 Hz, 1H), 7.03 (d, J=2.8 Hz, 1H), 5.19-5.32 (d, J=50.4 Hz, 1H), 3.95-3.97 (m, 4H), 3.90 (s, 2H), 3.81-3.83 (m, 8H), 3.01 (d, J=10.4 Hz, 1H), 2.81-2.86 (m, 6H), 2.39 (s, 3H), 2.04 (d, J=12.8 Hz, 1H), 1.83-1.89 (m, 1H)

### Example 83

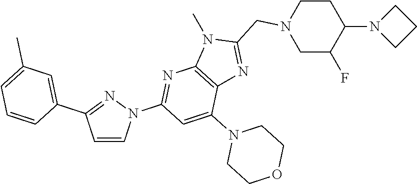 [SMILES: Cc1cccc(-c2ccn(-c3cc(N4CCOCC4)c4nc(CN5CCC(N6CCC6)C(F)C5)n(C)c4n3)n2)c1]

### Step 9: 4-(2-((4-(azetidin-1-yl)-3-fluoropiperidin-1-yl)methyl)-3-methyl-5-(3-(m-tolyl)-1H-pyrazol-1-yl)-3H-imidazo[4,5-b]pyridin-7-yl)morpholine

According to the procedure of Example 72, 4-(2-(chloromethyl)-5-(3-(m-tolyl)-1H-pyrazol-1-yl)thieno[3,2-b]pyridin-7-yl)morpholine (50 mg) is reacted with 4-(azetidin-1-yl)-3-fluoropiperidine (2 eq.) to provide a solid which is purified by silica gel column chromatography, to afford the title compound (9 mg; 14%) as an off-white solid. Mass [m/z]=544.7 [M+H]⁺; ¹H-NMR (400 MHz, CDCl₃): δ 8.62 (d, J=2.8 Hz, 1H), 7.78 (s, 1H), 7.72 (d, J=7.6 Hz, 1H), 7.31-7.34 (m, 2H), 7.15 (d, J=8.0 Hz, 1H), 6.75 (d, J=2.4 Hz, 1H), 4.50-4.68 (d, J=47 Hz), 3.99-4.00 (m, 4H), 3.93-3.95 (m, 4H), 3.80 (s, 2H), 3.29 (m, 4H), 3.11 (m, 2H), 2.82 (d, J=10.8 Hz, 2H), 2.45 (s, 4H), s·24 (d, J=14.4, 1H), 2.44 (t, J=11.2 Hz, 2H), 2.12 (t, J=6.8 Hz, 2H), 1.72 (d, J=14.4 Hz, 2H).

### Example 84

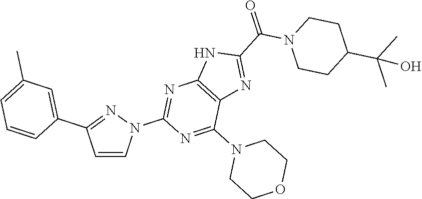 [SMILES: Cc1cccc(-c2ccn(-c3nc(N4CCOCC4)c4nc(C(=O)N5CCC(C(C)(C)O)CC5)[nH]c4n3)n2)c1]

### Step 4: (4-(2-hydroxypropan-2-yl)piperidin-1-yl)(6-morpholino-2-(3-(m-tolyl)-1H-pyrazol-1-yl)-9H-purin-8-yl)methanone

According to the procedure of Example 78, 6-morpholino-2-(3-(m-tolyl)-1H-pyrazol-1-yl)-9H-purine-8-carboxylic acid (20 mg, 0.04 mmol, 0.1 eq.) is reacted with 2-(piperidin-4-yl)propan-2-ol (9.8 mg, 0.05 mmol, 1.2 eq), propylphosphonic anhydride (50% in EtOAc) (62 mg, 0.09 mmol, 2 eq), and triethylamine (10 mg, 0.09 mmol, 2 eq), to provide a solid which is purified by Flash RP (C18 reverse phase 6 g column) eluting with 55-60% acetonitrile/water to afford the title compound (5 mg; 19%) as a white solid. Mass m/z 476.2 [M+H]⁺; ¹H-NMR (400 MHz, DMSO-d₆): 13.86 (br s, 1H), 8.70 (d, J=2.8 Hz, 1H), 7.77 (s, 1H), 7.72 (d, J=8.0 Hz, 1H), 7.35 (t, J=7.6 Hz, 1H), 7.19 (d, J=7.6 Hz, 1H), 7.06 (d, J=2.4 Hz, 1H), 5.04 (d, J=13.2 Hz, 1H), 4.60 (d, J=12.8 Hz, 1H), 4.30 (br s, 4H), 4.15 (s, 1H), 3.78 (t, J=4.8 Hz, 4H), 3.09 (t, J=12.8 Hz, 1H), 2.73-2.66 (m, 1H), 2.33 (s, 3H), 1.86-1.77 (m, 2H), 1.52-1.49 (m, 1H), 1.28-1.18 (m, 2H), 1.05 (s, 6H).

### Example 85

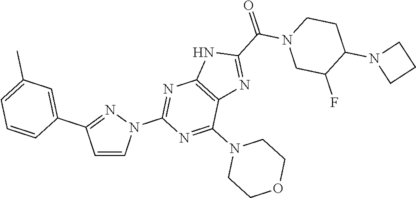 [SMILES: Cc1cccc(-c2ccn(-c3nc(N4CCOCC4)c4nc(C(=O)N5CCC(N6CCC6)C(F)C5)[nH]c4n3)n2)c1]

### Step 4: (4-(Azetidin-1-yl)-3-fluoropiperidin-1-yl)(6-morpholino-2-(3-(m-tolyl)-1H-pyrazol-1-yl)-9H-purin-8-yl)methanone

According to the procedure of Example 78, 6-morpholino-2-(3-(m-tolyl)-1H-pyrazol-1-yl)-9H-purine-8-carboxylic acid (35 mg, 0.086 mmol) in DCM (1 mL), is treated with 4-(azetidin-1-yl)-3-fluoropiperidine (9.8 mg, 0.10 mmol, 1.2 eq), propylphosphonic anhydride (50% in EtOAc) (62 mg, 0.17 mmol, 2 eq), and triethylamine (10 mg, 0.17 mmol, 2 eq), to provide a solid which is purified by Flash RP (C18 reverse phase 6 g column) using 55-60% acetonitrile/water as an eluent to afford the title compound (5 mg; 10%) as a white solid. Mass [m/z]=392.2 [M+H]⁺; ¹H-NMR (400 MHz, DMSO-d₆): 8.65 (d, J=2.8 Hz, 1H), 7.75 (s, 1H), 7.71 (d, J=8.0 Hz, 1H), 7.35 (t, J=7.6 Hz, 1H), 7.19 (d, J=7.6 Hz, 1H), 6.93 (d, J=2.4 Hz, 1H), 5.1 (d, J=112 Hz, 1H), 4.30 (s, 2H), 4.19 (t, 4H), 3.81 (t, J=4.8 Hz, 4H), 3.36 (br s, 2H), 3.12 (d, J=7.2 Hz, 1H), 2.04 (d, 1H), 1.71 (br s, 1H), 1.25-1.30 (m, 3H).

### Example 86

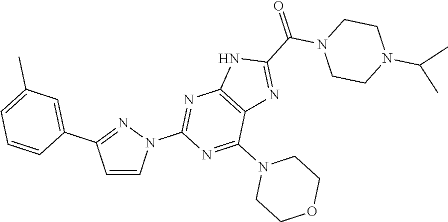 [SMILES: Cc1cccc(-c2ccn(-c3nc(N4CCOCC4)c4nc(C(=O)N5CCN(C(C)C)CC5)[nH]c4n3)n2)c1]

### Step 4: 4-Isopropylpiperazin-1-yl)(6-morpholino-2-(3-(m-tolyl)-1H-pyrazol-1-yl)-9H-purin-8-yl)methanone

According to the procedure of Example 78, 6-morpholino-2-(3-(m-tolyl)-1H-pyrazol-1-yl)-9H-purine-8-carboxylic acid (50 mg, 0.1 mmol) in DCM (1 mL), is treated with 1-isopropylpiperazine (20 mg, 0.12 mmol, 1.2 eq), propylphosphonic anhydride (50% in EtOAc) (62 mg, 0.09 mmol, 2 eq), and triethylamine (0.1 mL, 0.09 mmol, 2 eq), to provide a solid which is purified by Flash RP (C18 reverse phase 6 g column) using acetonitrile/water as an eluent to afford the title compound (4 mg; 14%) as a white solid. Mass [m/z]=392.2 [M+H]⁺; ¹H-NMR (400 MHz, DMSO-d₆): 13.86 (br s, 1H), 8.72 (s, J=2.4 Hz, 1H), 7.77 (s, 1H), 7.72 (d, J=8.0 Hz, 1H), 7.35 (t, J=7.6 Hz, 1H), 7.19 (d, J=7.6 Hz, 1H), 7.02 (d, J=2.4 Hz, 1H), 4.38 (br s, 1H), 3.79 (t, J=4.4 Hz, 4H), 3.66 (br s, 2H), 2.39 (s, 3H), 1.02 (s, 6H).

### Example 129

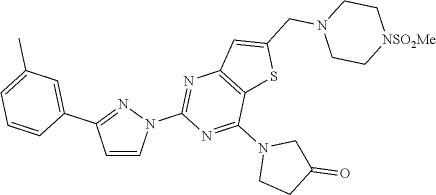

### Step 6: (2-chloro-4-(1,4-dioxa-7-azaspiro[4.4]nonan-7-yl)thieno[3,2-d]pyrimidin-6-yl)methanol

To a stirred solution of (2,4-dichlorothieno[3,2-d]pyrimidin-6-yl)methanol (100 mg, 0.39 mmol, 1 eq.) in IPA (4 mL) is added 1,4-dioxa-7-azaspiro[4.4]nonane (63 mg, 0.58 mmol, 1.5 eq) and DIPEA (0.15 mL, 0.85 mmol, 2 eq) at room temperature. The reaction mixture is heated to 80° C. and maintained for 12 h. The progress of the reaction is monitored by TLC. After completion of the reaction, it is diluted with EtOAc (2×30 mL) and washed with water (10 mL). Finally, the organic layer is dried over anhydrous Na₂SO₄, filtered and evaporated under reduced pressure to obtain a solid which was triturated with diethyl ether to afford the title compound (100 mg; 78%) as an off-white solid. Mass (m/z)=327.8 [M+H]⁺; ¹H-NMR (400 MHz, DMSO-d₆): 7.18 (s, 1H), 5.93 (t, J=6.0 Hz, 1H), 4.7-4.80 (m, 2H), 3.96-4.00 (m, 4H), 2.18 (br s, 2H), 1.17 (s, 3H).

### Step 7: 7-(2-chloro-6-(chloromethyl)thieno[3,2-d]pyrimidin-4-yl)-1,4-dioxa-7-azaspiro[4.4]nonane

To a stirred solution of (2-chloro-4-(1,4-dioxa-7-azaspiro[4.4]nonan-7-yl)thieno[3,2-d]pyrimidin-6-yl)methanol (100 mg, 0.31 mmol), in DCM (5 mL) at 0° C. is added SOCl₂ (0.1 mL). The reaction mass is allowed to warm to room temperature and stirred for 2 h. The progress of the reaction is monitored by TLC and after completion, it is evaporated under reduced pressure to afford the title compound (90 mg; 85%) as an off-white solid. Mass (m/z)=347.1 [M+H]⁺; ¹H-NMR (400 MHz, DMSO-d₆): 7.44 (s, 1H), 5.16 (s, 3H), 4.76 (s, 1H), 3.94-4.0 (m, 4H), 2.19 (br s, 2H), 1.23-1.33 (s, 3H).

### Step 8: 7-(2-chloro-6-((4-(methylsulfonyl)piperazin-1-yl)methyl)thieno[3,2-d]pyrimidin-4-yl)-1,4-dioxa-7-azaspiro[4.4]nonane

To a stirred solution of 7-(2-chloro-6-(chloromethyl)thieno[3,2-d]pyrimidin-4-yl)-1,4-dioxa-7-azaspiro[4.4]nonane (50 mg, 0.15 mmol) in IPA (3 mL) is added 1-(methylsulfonyl)piperazine (39 mg, 0.23 mmol, 1.5 eq) and DIPEA (0.06 mL, 0.39 mmol, 2 eq) at room temperature. The reaction mixture is heated to 80° C. and maintained for 12 h. The progress of the reaction is monitored by TLC. After completion of the reaction, it is diluted with EtOAc (2×25 mL) and washed with water (10 mL). Finally, the organic layer is dried over anhydrous Na₂SO₄, filtered and evaporated under reduced pressure to obtain a solid residue which is purified by a silica gel column chromatography (60-120 mesh) eluted with 40-50% EtOAc in hexane to afford the title compound (40 mg; 58%) as an off-white solid. Mass (m/z)=474.1 [M+H]⁺; ¹H-NMR (400 MHz, DMSO-d₆): 7.26 (s, 1H), 5.75 (s, 1H), 4.77 (s, 1H), 3.94-3.99 (m, 4H), 3.90 (s, 3H), 3.78 (m, 2H), 3.14 (t, J=4.8 Hz, 5H), 3.0 (s, 3H), 2.67 (t, J=4.8 Hz, 4H), 2.05 (br s, 2H), 1.02 (d, J=6.4 Hz, 1H).

### Step 9: 7-(6-((4-(methylsulfonyl)piperazin-1-yl)methyl)-2-(3-(m-tolyl)-1H-pyrazol-1-yl)thieno[3,2-d]pyrimidin-4-yl)-1,4-dioxa-7-azaspiro[4.4]nonane

To a solution of 7-(2-chloro-6-((4-(methylsulfonyl)piperazin-1-yl)methyl)thieno[3,2-d]pyrimidin-4-yl)-1,4-dioxa-7-azaspiro[4.4]nonane (40 mg 0.09 mmol) in (1:1) mixture of 1,4-dioxane:toluene (3 mL), is added 3-(m-tolyl)-1H-pyrazole (14 mg, 0.09 mmol, 1 eq), and K₃PO₄ (38 mg, 0.18 mmol, 2 eq). The reaction mixture is degassed for 10 min under nitrogen, followed by the addition of Pd₂(dba)₃ (8 mg, 0.009 mmol, 0.1 eq) and ^{t}Bu-XPhos (8 mg, 0.018 mmol, 0.2 eq) at room temperature. The reaction temperature is raised to 120° C. and stirred for 6 h. The progress of the reaction is monitored by TLC and after completion, it is diluted with EtOAc (25 mL) and washed with water (10 mL).

Finally, the organic layer is dried over anhydrous Na₂SO₄, filtered, and evaporated under reduced pressure to obtain a brownish gummy solid which is purified by RP Flash column chromatography (C18-6 g RP column) eluting with 60-65% acetonitrile/water, to afford after lyophilization the title compound (14 mg; 28%) as an off-white solid. Mass (m/z)=596.2 [M+H]⁺; ¹H-NMR (400 MHz, DMSO-d₆): 8.70 (d, J=2.4 Hz, 1H), 7.79 (Br s, 1H), 7.74 (d, J=8.0 Hz, 1H), 7.41 (s, 1H), 7.36 (t, J=7.6 Hz, 1H), 7.19 (d, J=7.6 Hz, 1H), 7.01 (d, J=2.8 Hz, 1H), 4.03-4.05 (m, 6H), 3.94 (s, 2H), 3.91 (s, 2H), 3.34 (s, 4H), 2.91 (s, 3H), 2.601 (Br s, 4H), 2.40 (s, 3H), 2.23 (t, J=7.2 Hz, 2H).

### Step 10: 1-(6-((4-(methylsulfonyl)piperazin-1-yl)methyl)-2-(3-(m-tolyl)-1H-pyrazol-1-yl)thieno[3,2-d]pyrimidin-4-yl)pyrrolidin-3-one

A solution of 7-(6-((4-(methylsulfonyl)piperazin-1-yl)methyl)-2-(3-(m-tolyl)-1H-pyrazol-1-yl)thieno[3,2-d]pyrimidin-4-yl)-1,4-dioxa-7-azaspiro[4.4]nonane (20 mg 0.036 mmol) in 0.4 mL dioxane at 0° C. is added a solution of 4N HCl in dioxane (0.5 mL) and the resulting mixture is brought to room temperature and stirred for 4 h. The progress of the reaction is monitored by TLC and after completion, it is diluted with EtOAc (25 mL) and washed with water (10 mL). Finally, the organic layer is dried over anhydrous Na₂SO₄, filtered, and evaporated under reduced pressure to obtain a brownish gummy material which is purified by Prep-HPLC purification using acetonitrile/water to afford the title compound (5 mg; 28%) as an off-white solid. Mass (m/z)=552.1 [M+H]⁺; ¹H-NMR (400 MHz, DMSO-d₆): 8.75 (d, J=2.4 Hz, 1H), 7.79 (br s, 1H), 7.42 (d, J=8.0 Hz, 1H), 7.50 (br s, 1H), 7.36 (t, J=7.6 Hz, 1H), 7.19 (d, J=7.6 Hz, 1H), 7.03 (d, J=2.8 Hz, 1H), 4.31-4.35 (m, 4H), 4.12 (br s, 2H), 3.21 (br s, 5H), 2.96 (s, 3H), 2.81 (t, J=6.0 Hz, 4H), 2.40 (s, 3H), 2.39 (s, 3H), 1.23 (s, 3H).

### Example 132

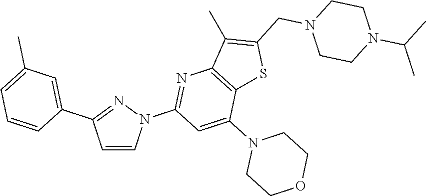 [SMILES: Cc1cccc(-c2ccn(-c3cc(N4CCOCC4)c4sc(CN5CCN(C(C)C)CC5)c(C)c4n3)n2)c1]

### Step 2: 4-(2-((4-Isoproylpiperazin-1-yl)methyl)-3-methyl-5-(3-(m-tolyl)-1H-pyrazol-1-yl)thieno[3,2-b]pyridin-7-yl)morpholine

Following the procedure according to Example 167, 4-(2-(chloromethyl)-3-methyl-5-(3-(m-tolyl)-1H-pyrazol-1-yl)thieno[3,2-b]pyridin-7-yl)morpholine (40 mg) is reacted with 1-isopropylpiperazine (17 mg) to afford the title compound (21 mg; 40%) as an off-white solid. Mass [m/z]=531 [M+H]⁺; ¹H-NMR (400 MHz, DMSO-d₆): 8.75 (d, J=2.4 Hz, 1H), 7.79-7.77 (br d, 2H), 7.43 (s, 1H), 7.36 (t, J 7.6 Hz, 1H), 7.19 (d, J=7.2 Hz, 1H), 7.05 (d, J=2.4 Hz, 1H), 3.85 (t, J=4.8 Hz, 4H), 3.78 (s, 2H), 3.50 (t, J=4.4 Hz, 4H), 3.3 (s, 4H), 2.4 (s, 6H).

### Example 165

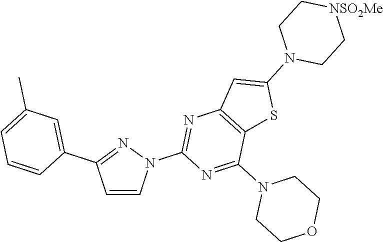

### Step 1: 4-(2-chloro-6-iodothieno[3,2-d]pyrimidin-4-yl)morpholine

To a stirred solution of 4-(2-chlorothieno[3,2-d]pyrimidin-4-yl)morpholine (500 mg, 1.96 mmol) in THE (8 mL) at −78° C. is added n-BuLi (1.0 M in THF; 2.94 mL, 2.94 mmol, 1.5 eq) and the reaction is stirred for 15 min, followed by the addition of iodochloroethane (2.94 mL, 2.94 mmol, 1.5 eq) at the same temperature. The reaction mixture is then brought to room temperature and stirred for another 4 h. The progress of the reaction is monitored by TLC, and after completion of the reaction, it is quenched with saturated NH₄Cl solution (25 mL) and extracted with EtOAc (2×60 mL). The organic layer is dried over Na₂SO₄, filtered, and evaporated under a vacuum to obtain an oil which is purified by silica gel chromatography (60-120 mesh) eluting with 10-15% EtOAc/hexane, to afford the title compound (300 mg; 40%) as an off-white solid. Mass (m/z)=382 [M+H]⁺; ¹H-NMR (400 MHz, DMSO-d₆): 7.25 (s, 1H), 3.90 (t, J=4.4 Hz, 4H), 3.74 (t, J=5.2 Hz, 4H).

### Step 2: 4-(2-chloro-6-(4-(methylsulfonyl)piperazin-1-yl)thieno[3,2-d]pyrimidin-4-yl)morpholine

To a stirred solution of 4-(2-chloro-6-iodothieno[3,2-d]pyrimidin-4-yl)morpholine (200 mg, 0.52 mmol) and 1-(methylsulfonyl)piperazine (172 mg, 1.04 mmol, 2.0 eq.) in dry DMF is added Cs₂CO₃ (164 mg, 0.46 mmol, 0.9 eq.). The reaction mixture is degassed for 15 min and then Pd₂(dba)₃ (91 mg, 0.1 mmol, 0.2 eq.) is added followed by Xantphos (115 mg, 0.2 mmol, 0.4 eq.). The resulting reaction mixture is allowed to stir at 110° C. for 12 h. After completion (monitored by TLC), the reaction mixture is diluted with EtOAc filtered and filtrate is concentrated. The obtained residue is purified by reverse phase flash chromatography (used C18, 12 g column) eluting with 50% to 60% acetonitrile in water to afford the title compound (55 mg; 25%) as an off-white solid. Mass (m/z)=418.1 [M+H]⁺; ¹H-NMR (400 MHz, DMSO-d₆): 6.37 (s, 1H), 3.71-3.75 (m, 8H), 3.50 (t, J=5.2 Hz, 4H), 3.26 (t, J=4.8 Hz, 4H), 2.93 (s, 3H).

### Step 3: 4-(6-(4-(methylsulfonyl)piperazin-1-yl)-2-(3-(m-tolyl)-1H-pyrazol-1-yl)thieno[3,2-d]pyrimidin-4-yl)morpholine

To a stirred solution of 4-(2-chloro-6-(4-(methylsulfonyl)piperazin-1-yl)thieno[3,2-d]pyrimidin-4-yl)morpholine (45 mg, 0.11 mmol), and 3-(m-tolyl)-1H-pyrazole (25 mg, 0.16 mmol, 1.5 eq.) dissolved in a mixture of toluene and dioxane (1:1, 4 mL) is added Pd₂(dba)₃ (9.2 mg, 0.01 mmol, 0.1 eq.), ^{t}Bu-XPhos (8.5 mg, 0.02 mmol, 0.2 eq.), and then K₃PO₄ (46 mg, 0.22 mmol, 2 eq.) and the mixture is degassed for 30 min. The reaction mixture is then stirred at 120° C. for 6 h. Progress of the reaction is monitored by TLC. After complete consumption of the starting material, the solids are filtered by passing the reaction mixture through a bed of celite. The filtrate is diluted with EtOAc (20 mL), and washed with water (10 mL). Finally, the organic layer is dried over anhydrous Na₂SO₄, filtered, and evaporated under reduced pressure to obtain a crude product as a brown liquid which is purified by silica gel chromatography (60-120 mesh) using 60%-65% ethyl acetate/petroleum ether as eluent to afford the title compound (10 mg; 53%) as an off white solid. Mass (m/z)=540.2 [M+H]⁺; ¹H-NMR (400 MHz, DMSO-d₆): 8.66 (d, J=2.8 Hz, 1H), 7.77 (s, 1H), 7.21 (d, 1H), 7.34 (m, 1H), 7.18 (d, J=3.6 Hz, 1H), 6.99 (d, J=2.8 Hz, 1H), 6.53 (s, 1H), 3.89 (t, J=5.2 Hz, 4H), 3.77 (t, J=4.4 Hz, 4H), 3.53 (t, J=5.2 Hz, 4H), 3.28 (s, 4H), 2.95 (s, 3H), 2.39 (s, 3H).

### Example 166

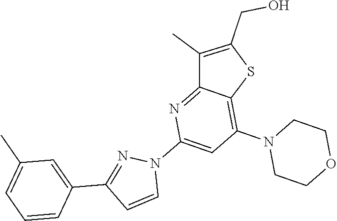 [SMILES: Cc1cccc(-c2ccn(-c3cc(N4CCOCC4)c4sc(CO)c(C)c4n3)n2)c1]

### Step 1: Ethyl-5,7-dihydroxy-3-methylthieno[3,2-b]pyridine-6-carboxylate

To a stirred solution of methyl 3-amino-4-methylthiophene-2-carboxylate (10 g, 58.47 mmol) in DCM (100 mL) is added triethylamine (11 mL, 76.03 mmol, 1.2 eq) and ethyl malonyl chloride (10 mL, 70.17 mmol, 1.3 eq) at room temperature and the reaction is stirred for 1 h. The reaction mixture is diluted with DCM (50 mL) and washed with brine solution (50 mL). The organic layer is dried over Na₂SO₄, filtered, and evaporated under vacuum to afford the title compound (16 g, ˜98%) as a brown sticky solid. Mass [m/z]=254 [M+H]⁺; ¹H-NMR (400 MHz, DMSO-d₆): 13.43 (br s, 1H), 11.78 (br s, 1H), 7.75 (s, 1H), 4.33 (q, J=7.2, 14.0 Hz, 2H), 2.24 (s, 3H), 1.30 (t, J 7.2 Hz, 3H).

### Step 2: 3-Methylthieno[3,2-b]pyridine-5,7-diol

To a stirred solution of ethyl 5,7-dihydroxy-3-methylthieno[3,2-b]pyridine-6-carboxylate (15 g, 59.28 mmol) in EtOH (60 mL) is added sodium ethoxide (1.2 g, 17.78 mmol, 0.3 eq) followed by 40% aqueous NaOH (100 mL) at room temperature. The reaction mixture is heated to 80° C. and stirred for 16 h. The reaction mixture is cooled to room temperature and the pH is adjusted up to 3 using 2N aqueous HCl. The precipitated solid is collected and dried by vacuum filtration to afford the title compound (7.5 g; 75%) as a brown solid. Mass [m/z]=182 [M+H]⁺; ¹H-NMR (400 MHz, DMSO-d₆): 11.84 (br s, 2H), 7.51 (d, J=8.0 Hz, 1H), 5.79 (s, 1H), 2.25 (s, 3H).

### Step 3: 5,7-Dichloro-3-methylthieno[3,2-b]pyridine

3-Methylthieno[3,2-b]pyridine-5,7-diol (7 g, 38.67 mmol) is dissolved in neat POCl₃ (70 mL), at room temperature. The reaction is stirred at 110° C. for 24 h and then it is concentrated under reduced pressure. The resulting residue is quenched with ice cold water (100 mL) and stirred for 15 min to a give solid which is filtered and dried to afford the title compound (5.2 g; 62%) as a brown solid. Mass [m/z]=218 [M+H]⁺; ¹H-NMR (400 MHz, CDCl₃): 8.75 (s, 1H), 7.79 (br s, 1H), 7.75 (d, J=7.6 Hz, 1H), 7.36 (t, J=11.4 Hz 1H), 7.2 (d, J=7.6 Hz, 1H), 7.05 (d, J=2.8 Hz, 1H), 4.06 (t, J=7 Hz 4H), 3.83 (t, J=4.8 Hz, 4H), 2.39 (s, 3H), 2.33-2.32 (m, 4H), 1.23 (s, 1H)

### Step 4: 4-(5-Chloro-3-methylthieno[3,2-b]pyridin-7-yl)morpholine

To a stirred solution of 5,7-dichloro-3-methylthieno[3,2-b]pyridine (5 g, 23.04 mmol) in ethylene glycol (50 mL) at room temperature is added morpholine (9 mL, 92.16 mmol, 4 eq). The reaction mixture is stirred at 110° C. for 16 h. The reaction mixture is cooled to room temperature, diluted with EtOAc and washed with water. The organic layer is dried over Na₂SO₄, filtered, and evaporated under vacuum to obtain a crude solid which is purified by silica gel column chromatography (60-120 mesh) eluting with 8-10% EtOAc/hexane to afford the title compound (3.2 g; 53%) as a pale-yellow solid. Mass [m/z]=218 [M+H]⁺; ¹H-NMR (400 MHz, CDCl₃): 6.83 (s, 1H), 3.91 (t, J=4.4 Hz, 4H), 3.76 (t, J=5.2 Hz, 4H).

### Step 5: 5-Chloro-3-methyl-7-morpholinothieno[3,2-b]pyridine-2-carbaldehyde

To a stirred solution of 4-(5-chloro-3-methylthieno[3,2-b]pyridin-7-yl)morpholine (3.5 g, 13.05 mmol) in dry THE (30 mL) is cooled to −78° C. and n-BuLi (2.5 M in THF) (10.4 mL, 26.11 mmol, 2 eq) added followed by DMF (6 mL, 78.35 mmol, 6 eq). The reaction mixture is stirred at −78° C. for an additional 3 h. and then it is quenched with saturated aqueous NH₄Cl solution (30 mL). The resulting solid is filtered, dried, and triturated with diethyl ether to afford the title compound (3.2 g; 84%) as a pale-yellow solid. Mass [m/z]=297.0 [M+H]⁺; ¹H-NMR (400 MHz, CDCl₃): 3.43 (t, J=3.6 Hz, 1H), 7.82 (d, J=9.2 Hz, 2H), 7.38 (t, J=7.6 Hz, 1H), 7.24 (d, J=7.6 Hz, 1H), 1.17 (d, J=2.8 Hz, 1H), 7.15 (s, 1H), 3.93 (t, J=4.0 Hz, 4H), 3.84 (t, J=4.8 Hz, 4H), 2.40 (s, 3H).

### Step 6: (5-Chloro-3-methyl-7-morpholinothieno[3,2-b]pyridin-2-yl)methanol

To a stirred solution of 5-chloro-3-methyl-7-morpholinothieno[3,2-b]pyridine-2-carbaldehyde (3 g, 10.13 mmol) in THE (30 mL) at 0° C. is added NaBH₄ (0.770 g, 20.27 mmol, 2 eq) and the reaction is stirred for 2 h. The reaction mixture is quenched with cold water (50 mL) and extracted with EtOAc (2×50 mL). The organic layer is dried over Na₂SO₄, filtered, and evaporated under vacuum to obtain a solid which is triturated with n-pentane to afford the title compound (2.8 g; 93%) as a yellow solid. Mass [m/z]=299 [M+H]⁺; ¹H-NMR (400 MHz, CDCl₃): 6.63 (s, 1H), 4.96 (d, J=5.2 Hz, 2H), 3.90 (t, J=4.2 Hz, 4H), 3.44 (t, J=4.8 Hz, 4H), 2.40 (s, 3H), 2.0 (t, J=5.6 Hz, 1H).

### Step 7: (3-Methyl-7-morpholino-5-(3-(m-tolyl)-1H-pyrazol-1-yl)thieno[3,2-b]pyridin-2-yl)methanol

To a stirred solution of (5-chloro-3-methyl-7-morpholinothieno[3,2-b]pyridin-2-yl)methanol (1 g, 3.34 mmol), and 3-(m-tolyl)-1H-pyrazole (0.530 g, 3.34 mmol, 1 eq.) in a mixture of toluene and dioxane (1:1) (10 mL) is added Pd₂(dba)₃ (0.307 g, 0.03 mmol, 0.1 eq.), t-BuXphos (0.284 g, 0.668 mmol, 0.2 eq.), and then K₃PO₄ (1.4 g, 6.68 mmol, 2 eq.). The mixture is degassed with nitrogen for 30 min, then it is heated to 120° C. and stirred for 16 h. The reaction mixture is filtered through a celite pad, and the filtrate is diluted with EtOAc (30 mL). The organic layer is washed with water (10 mL), dried over anhydrous Na₂SO₄, filtered, and evaporated under reduced pressure to obtain a brown liquid, which is purified by reverse phase chromatography (C18 reverse phase 12 g column), using 70-80% acetonitrile/water as an eluent, to afford the title compound (220 mg; 14%) as an off white solid. Mass [m/z]=421.2 [M+H]⁺; ¹H-NMR (400 MHz, DMSO-d₆): 8.76 (d, J=2.8 Hz, 1H), 7.80-7.70 (m, 2H), 7.43 (s, 1H), 7.36 (t, J=7.6 Hz, 1H), 7.20 (d, J=7.6 Hz, 1H), 7.05 (d, J=2.8 Hz, 1H), 5.73 (t, J=5.6 Hz, 1H), 4.80 (d, J=5.2 Hz, 2H), 3.85 (t, J=4.4 Hz, 4H), 3.52 (t, J=4.4 Hz, 4H), 2.44 (s, 3H), 2.40 (s, 3H).

### Example 167

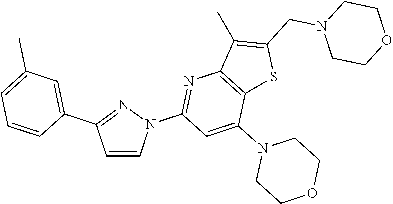 [SMILES: Cc1cccc(-c2ccn(-c3cc(N4CCOCC4)c4sc(CN5CCOCC5)c(C)c4n3)n2)c1]

### Step 1: 4-(2-(Chloromethyl)-3-methyl-5-(3-(m-tolyl)-1H-pyrazol-1-yl)thieno[3,2-b]pyridin-7-yl)morpholine

To (3-methyl-7-morpholino-5-(3-(m-tolyl)-1H-pyrazol-1-yl)thieno[3,2-b]pyridin-2-yl)methanol (200 mg, 3.34 mmol) at 0° C. is added SOCl₂ (0.1 mL), and the reaction is warmed to rt and stirred for 2 h. The reaction mixture is evaporated under reduced pressure to provide an off-white solid which is purified by flash chromatography (C18 reverse phase 12 g column) using 70-80% acetonitrile/water as an eluent, to afford the title compound (220 mg; 96%) as an off white solid. Mass [m/z]=439.2 [M+H]⁺; ¹H-NMR (400 MHz, DMSO-d₆): 8.78 (d, J=2.4 Hz, 1H), 7.80-7.78 (m, 2H), 7.49 (s, 1H), 7.36 (t, J=7.6 Hz, 1H), 7.21-7.19 (d, J=7.6 Hz, 1H), 7.07 (d, J=2.4 Hz, 1H), 5.176 (s, J=2H), 3.85 (t, J=4.4 Hz, 4H), 3.52 (t, J=4.4 Hz, 4H), 2.46 (s, 3H), 2.40 (s, 3H).

### Step 2: 4-(3-Methyl-2-(morpholinomethyl)-5-(3-(m-tolyl)-1H-pyrazol-1-yl)thieno[3,2-b]pyridin-7-yl)morpholine

To the stirred solution of 4-(2-(chloromethyl)-3-methyl-5-(3-(m-tolyl)-1H-pyrazol-1-yl)thieno[3,2-b]pyridin-7-yl)morpholine (30 mg, 1.0 equiv.) in dry THE (10 vol) at room temperature is added morpholine (20.5 mg, 1.5 equiv.) followed by K₂CO₃ (2.5 equiv.). The reaction mixture is heated to 80° C. and allowed to stir for 16 h. The progress of the reaction is monitored by TLC. The reaction is then diluted with water (10 vol) and extracted with ethyl acetate (2×10 vol). The combined organic layers are dried over anhydrous Na₂SO₄, filtered and evaporated under reduced pressure to obtain a crude product which is purified by flash RP column chromatography using water/acetonitrile or by recrystallization to provide the title compound (13 mg; 40%) as an off-white solid. Mass [m/z]=490 [M+H]⁺; ¹H-NMR (400 MHz, DMSO-d₆): 8.75 (d, J=3.2 Hz, 1H), 7.79-7.76 (br d, 2H), 7.43 (s, 1H), 7.38 (t, J 7.6 Hz, 1H), 7.19 (d, J=7.2 Hz, 1H), 7.05 (d, J=2.4 Hz, 1H), 3.85 (t, J=4.8 Hz, 4H), 3.80 (s, 2H), 3.60 (t, J=4.0 Hz, 4H), 3.50 (t, J=4.4 Hz, 4H), 3.3 (s, 4H), 2.4 (s, 6H).

### Example 168

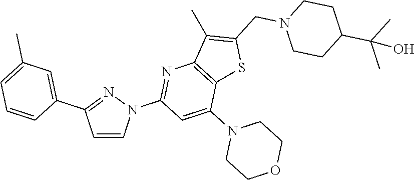 [SMILES: Cc1cccc(-c2ccn(-c3cc(N4CCOCC4)c4sc(CN5CCC(C(C)(C)O)CC5)c(C)c4n3)n2)c1]

### Step 2: 2-(1-((3-Methyl-7-morpholino-5-(3-(m-tolyl)-1H-pyrazol-1-yl)thieno[3,2-b]pyridin-2-yl)methyl)piperidin-4-yl)propan-2-ol

Following the procedure according to Example 167, 4-(2-(chloromethyl)-3-methyl-5-(3-(m-tolyl)-1H-pyrazol-1-yl)thieno[3,2-b]pyridin-7-yl)morpholine (40 mg) is reacted with 2-(piperidin-4-yl)propan-2-ol (19 mg) to afford the title compound (26 mg; 54%) as an off-white solid. Mass [m/z]=531 [M+H]⁺; ¹H-NMR (400 MHz, DMSO-d₆): 8.78 (d, J=2.8 Hz, 1H), 7.79 (s, 1H), 7.79-7.82 (s, 2H), 7.46 (s, 1H), 7.38 (t, J=7.2 Hz, 1H), 7.21 (d, J=7.6 Hz, 1H), 7.08 (d, J=2.4 Hz, 1H), 4.09 (s, 1H), 3.87 (t, J=3.2 Hz, 4H), 3.77 (s, 1H), 3.52 (s, 4H), 3.01 (s, 2H), 2.42 (s, 6H), 1.99 (s, 2H), 1.68 (s, 2H), 1.25 (br m, 3H), 1.06 (s, 6H).

### Example 169

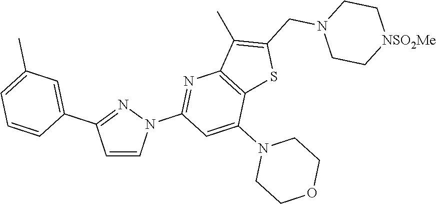

### Step 2: 4-(3-Methyl-2-((4-(methylsulfonyl)piperazin-1-yl)methyl)-5-(3-(m-tolyl)-1H-pyrazol-1-yl)thieno[3,2-b]pyridin-7-yl) morpholine

Following the procedure according to Example 167, 4-(2-(chloromethyl)-3-methyl-5-(3-(m-tolyl)-1H-pyrazol-1-yl)thieno[3,2-b]pyridin-7-yl)morpholine (40 mg) is reacted with 1-(methyl sulfonyl) piperazine (22 mg) to afford the title compound (30 mg; 54%) as an off-white solid. Mass [m/z]=546 [M+H]⁺; ¹H-NMR (400 MHz, DMSO-d₆): 8.76 (d, J=2.84 Hz, 1H), 7.77 (br s, 2H), 7.44 (s, 1H), 7.38 (s, 1H), 7.36 (t, J=7.6 Hz, 1H), 7.19 (d, J=7.6 Hz, 1H), 7.05 (d, J=3.2 Hz, 1H), 3.87 (s, 2H), 3.85 (t, J=5.2 Hz, 4H), 3.50 (t, J=4.4 Hz, 4H), 3.14 (t, J=4.8 Hz, 4H), 2.9 (s, 3H), 2.67 (t, J=1.6 Hz, 4H), 2.32 (s, 6H).

### Example 170

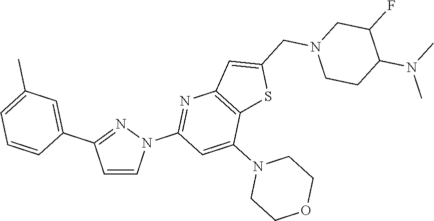 [SMILES: Cc1cccc(-c2ccn(-c3cc(N4CCOCC4)c4sc(CN5CCC(N(C)C)C(F)C5)cc4n3)n2)c1]

### Step 2: 3-Fluoro-N,N-dimethyl-1-((3-methyl-7-morpholino-5-(3-(m-tolyl)-1H-pyrazol-1-yl)thieno[3,2-b]pyridin-2-yl)methyl)piperidin-4-amine

Following Step 1 of Example 167, 4-(2-(chloromethyl)-3-methyl-5-(3-(m-tolyl)-1H-pyrazol-1-yl)thieno[3,2-b]pyridin-7-yl)morpholine (40 mg, 1.0 equiv.) in IPA (10 vol) at room temperature is added 3-fluoro-N, N-dimethyl-piperidin-4-amine (20 mg, 2.0 eq.) followed by DIPEA (2.0 eq.). The reaction mixture is heated to 80° C. and stirred for 16 h. The reaction mixture is concentrated under reduced pressure, diluted with water (10 vol), and extracted with EtOAc (2×10 vol). The combined organic layers are dried over anhydrous Na₂SO₄, filtered, and evaporated under reduced pressure to obtain a crude compound which is purified by reverse phase flash chromatography to provide the title compound (26 mg; 50%) as an off-white solid. Mass [m/z]=549 [M+H]⁺; ¹H-NMR (400 MHz, DMSO-d₆): 8.76 (d, J=2.4 Hz, 1H), 7.80 (s, 1H), 7.77 (s, 1H), 7.44 (s, 1H), 7.36 (t, J=7.6 Hz, 1H), 7.19 (d, J=7.6 Hz, 1H), 4.85-4.97 (d, 1H), 3.83-3.86 (m, 6H), 3.57 (t, J=4.4 Hz, 4H), 3.16-3.19 (m, 2H), 2.96 (d, 1H), 2.39 (d, J=3.6 Hz, 6H), 2.36 (m, 1H), 2.30 (m, 2H), 1.74 (m, 1H), 1.65 (m, 1H).

### Example 171

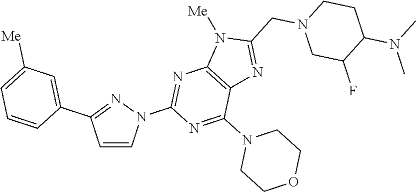 [SMILES: Cc1cccc(-c2ccn(-c3nc(N4CCOCC4)c4nc(CN5CCC(N(C)C)C(F)C5)n(C)c4n3)n2)c1]

### Step 11: 3-fluoro-N,N-dimethyl-1-((9-methyl-6-morpholino-2-(3-(m-tolyl)-1H-pyrazol-1-yl)-9H-purin-8-yl)methyl)piperidin-4-amine

According to the procedure of Example 56, 4-(8-(chloromethyl)-9-methyl-2-(3-(m-tolyl)-1H-pyrazol-1-yl)-9H-purin-6-yl)morpholine (30 mg, 0.07 mmol) in IPA (3 mL) is treated with 3-fluoro-N,N-dimethylpiperidin-4-amine (20.6 mg, 0.14 mmol, 2 eq) and DIPEA (530 mg, 0.21 mmol, 3 eq) at room temperature, and the obtained brown gummy solid is purified by flash RP chromatography (C18, 6 g column) using water/acetonitrile as eluent, to afford the title compound (7 mg; 30%) as an off-white solid. Mass [m/z]=534.3 [M+H]⁺; ¹H-NMR (400 MHz, DMSO-d₆): 9.70 (s, 1H), 8.72 (d, J=2.8 Hz, 1H), 7.77 (s, 1H), 7.74 (d, J=8.0 Hz, 1H), 7.35 (t, J=7.6 Hz, 1H), 7.19 (d, J=7.6 Hz, 1H), 7.01 (d, J=2.4 Hz, 1H), 5.19-5.32 (d, J=49.2 Hz, 1H), 4.29 (br s, 4H), 3.89 (s, 2H), 3.78-3.82 (m, 7H), 3.59-3.42 (m, 2H), 3.44 (t, J=5.6 Hz, 1H), 3.02 (d, J=10.4 Hz, 1H), 2.82-2.85 (dd, J=8 Hz, 8.4 Hz, 6H), 2.39 (s, 3H), 2.32 (t, J=1.6 Hz, 1H), 2.04 (m, 1H), 1.84-1.90 (m, 1H).

### Example 172

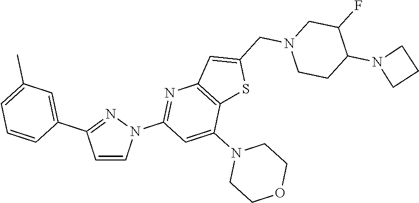 [SMILES: Cc1cccc(-c2ccn(-c3cc(N4CCOCC4)c4sc(CN5CCC(N6CCC6)C(F)C5)cc4n3)n2)c1]

### Step 2: 4-(2-((4-(Azetidin-1-yl)-3-fluoropiperidin-1-yl)methyl)-3-methyl-5-(3-(m-tolyl)-1H-pyrazol-1-yl)thieno[3,2-b]pyridin-7-yl)morpholine

Following the procedure according to Example 170, 4-(2-(chloromethyl)-3-methyl-5-(3-(m-tolyl)-1H-pyrazol-1-yl)thieno[3,2-b]pyridin-7-yl)morpholine is reacted with 4-(azetidin-1-yl)-3-fluoropiperidine (22 mg) to afford the title compound (15 mg; 34%) as an off-white solid. Mass [m/z]=551 [M+H]⁺; ¹H-NMR (400 MHz, DMSO-d₆): 8.76 (d, J=5.6 Hz, 1H), 7.80 (s, 1H), 7.77 (s, 1H), 7.46 (s, 1H), 7.36 (t, J=7.2 Hz, 1H), 7.06 (d, J=3.2 Hz, 1H), 4.99-5.11 (d, 1H), 4.19-4.29 (m, 1H) 4.02 (br s, 4H), 3.83 (s, 4H), 3.51 (s, 4H), 3.08-3.11 (m, 1H), 2.42 (s, 6H), 2.24 (br m, 2H), 1.87 (s, 1H), 1.60-1.70 (m, 1H).

### Example 173

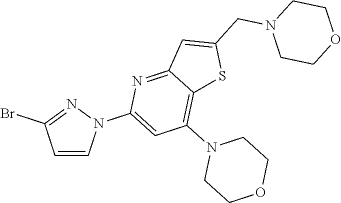 [SMILES: Brc1ccn(-c2cc(N3CCOCC3)c3sc(CN4CCOCC4)cc3n2)n1]

### Step 1: 4-(5-Chlorothieno[3,2-b]pyridin-7-yl)morpholine

To a stirred solution of 5,7-dichlorothieno[3,2-b]pyridine (3.0 g, 14.7 mmol) in ethylene glycol (10 mL) is added morpholine (2.5 mL, 29.41 mmol, 2.0 eq.), followed by TEA (4.1 mL, 29.41 mmol, 2.0 eq.), at room temperature and then the reaction is heated to 120° C. and stirred for 16 h. The reaction mixture is diluted with H₂O (30 mL) and the resulting solid is washed with water, dried and then triturated with diethyl ether to afford the title compound (2.6 g, 70%) as a brown solid. Mass [m/z]=255 [M+H]⁺; ¹H-NMR (400 MHz, DMSO-d₆): 7.66 (d, J=5.6 Hz, 1H), 7.44 (d, J=5.6 Hz, 1H), 6.65 (s, 1H), 3.92 (t, J=4.4 Hz, 4H), 3.47 (t, J=4.8 Hz, 4H).

### Step 2: 5-Chloro-7-morpholinothieno[3,2-b]pyridine-2-carbaldehyde

To stirred THE (50 mL) at −78° C. is added n-BuLi (2.5 M) (17 mL, 44.2 mmol, 2.5 equiv.) dropwise and the mixture is stirred for 10 min, followed by the addition of a solution of 4-(5-chlorothieno[3,2-b]pyridin-7-yl)morpholine (4.5 g, 17.7 mmol, 1.0 eq.) in THE (30 mL). The resulting mixture is stirred for 45 min at −78° C., then dry DMF (8.5 mL, 106.2 mmol, 6.0 equiv.) is added dropwise. The reaction mixture is stirred for a further 20 min at −78° C. after which the resulting mixture is allowed to warm to 0° C., and then it is quenched with water and extracted with EtOAc (2×50 mL). The combined organic layers are washed with 1 N HCl (50 mL), followed by brine (50 mL), dried over Na₂SO₄, and concentrated under reduced pressure to provide a crude yellow solid which is triturated with diethyl ether to afford the title compound (3.5 g, 71%) as a yellow solid. Mass [m/z]=283 [M+H]⁺; ¹H-NMR (400 MHz, CDCl₃): 10.16 (s, 1H), 8.07 (s, 1H), 6.74 (s, 1H), 3.93 (t, J=4.8 Hz, 4H), 3.49 (t, J=4.8 Hz, 4H).

### Step 3: (5-Chloro-7-morpholinothieno[3,2-b]pyridin-2-yl)methanol

To a stirred solution of 5-chloro-7-morpholinothieno[3,2-b]pyridine-2-carbaldehyde (3.5 g, 12.41 mmol) in MeOH:DCM (1:1, 40 mL) at 0° C. is added NaBH₄ (1.0 g, 24.8 mmol, 2.0 eq.) portionwise. The reaction mixture is stirred at RT for 3 h, cooled to 0° C., then quenched with water and extracted with EtOAc (2×50 mL). The combined organic layers are washed with brine (50 mL), dried over Na₂SO₄, and concentrated under reduced pressure producing a brown solid which is triturated with diethyl ether to afford the title compound (2.2 g, 62%) as a white solid. Mass [m/z]=285 [M+H]⁺; ¹H-NMR (400 MHz, DMSO-d₆): 7.27 (s, 1H), 6.61 (s, 1H), 4.96 (s, 2H), 3.90 (t, J=4.4 Hz, 4H), 3.45 (t, J=4.8 Hz, 4H), 2.42 (d, J=7.6 Hz, 1H).

### Step 4: 4-(5-Chloro-2-(chloromethyl)thieno[3,2-b]pyridin-7-yl)morpholine

To a stirred solution of (5-chloro-7-morpholinothieno[3,2-b]pyridin-2-yl)methanol (2.0 g, 7.04 mmol) in DCM (20 mL) at 0° C. is added SOCl₂ (2.5 mL, 21.05 mmol, 3.0 equiv.) at 0° C. The reaction mixture is stirred and warmed to RT and then is stirred for 4 h. The reaction mixture is concentrated under reduced pressure and the resulting solid is triturated with n-pentane to afford the title compound (1.7 g, 80%) as an off-white solid. Mass [m/z]=303 [M+H]⁺; ¹H-NMR (400 MHz, CDCl₃): 8.11 (s, 1H), 6.66 (s, 1H), 4.86 (s, 2H), 3.97 (br s, 4H), 3.84 (br s, 4H).

### Step 5: 4-(5-Chloro-2-(morpholinomethyl)thieno[3,2-b]pyridin-7-yl)morpholine

To a stirred solution of 4-(5-chloro-2-(chloromethyl)thieno[3,2-b]pyridin-7-yl)morpholine (1.4 g, 4.63 mmol) in THE (20 mL) is added morpholine (1.3 mL, 13.89 mmol, 3.0 eq.) at RT, followed by K₂CO₃ (1.2 g, 9.26 mmol, 2.0 eq.). The reaction temperature is raised to 80° C. and the reaction is stirred for 16 h. After completion of the reaction, it is cooled and diluted with water (25 mL). The product is extracted with EtOAc (2×25 mL) and the combined organic layers are washed with brine (20 mL), dried over Na₂SO₄, then concentrated under reduced pressure to provide a solid which is triturated with diethyl ether to afford the title compound (1.1 g, 68%) as an off white solid. Mass [m/z]=354 [M+H]⁺; ¹H-NMR (400 MHz, CDCl₃): 7.24 (s, 1H), 6.60 (s, 1H), 3.92 (t, J=4.4 Hz, 4H), 3.79 (s, 2H), 3.74 (t, J=4.4 Hz, 4H), 3.44 (t, J=4.8 Hz, 4H), 2.54 (t, J=4.4 Hz, 4H).

### Step 6: 4-(5-(3-Bromo-1H-pyrazol-1-yl)-2-(morpholinomethyl)thieno[3,2-b]pyridin-7-yl)morpholine

To a stirred solution of 4-(5-chloro-2-(morpholinomethyl)thieno[3,2-b]pyridin-7-yl)morpholine (1.1 g, 3.10 mmol) in N-methylpyrrolidinone (10 mL) is added bromopyrazole (1.37 g, 9.32 mmol, 3.0 eq.), followed by Cs₂CO₃ (2.0 g, 6.21 mmol, 2.0 eq.) at RT. The reaction is heated to 210° C. and stirred for 20 h, then cooled and diluted with ice water and the resulting solid is filtered, washed with cold water and dried under vacuum to obtain a crude product. The crude product is triturated with diethyl ether to afford the title compound (0.75 g, 53%) as a pale brown solid. Mass [m/z]=464 [M+H]⁺; ¹H-NMR (400 MHz, DMSO-d₆): 8.52 (d, J=2.4 Hz, 1H), 7.34 (s, 1H), 7.26 (s, 1H), 6.47 (d, J=2.4 Hz, 1H), 3.93 (t, J=4.4 Hz, 4H), 3.81 (s, 2H), 3.75 (t, J=4.4 Hz, 4H), 3.53 (t, J=4.8 Hz, 4H), 2.56-2.57 (br s, 4H).

### Example 174

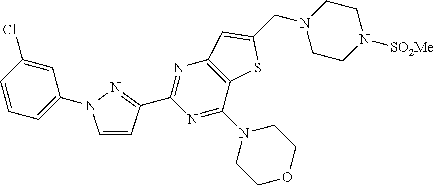 [SMILES: CS(=O)(=O)N1CCN(Cc2cc3nc(-c4ccn(-c5cccc(Cl)c5)n4)nc(N4CCOCC4)c3s2)CC1]

### Step 1: 4-(2-Chloro-6-(chloromethyl)thieno[3,2-d]pyrimidin-4-yl)morpholine

To a stirred solution of (2-chloro-4-morpholinothieno[3,2-d]pyrimidin-6-yl)methanol (500 mg, 1.74 mmol) in DCM (10 mL) at 0° C. is added SOCl₂ (0.4 mL). The reaction is allowed to reach room temperature and is stirred for 4 h. The reaction mixture is evaporated under reduced pressure to afford the title compound (490 mg; 90%) as a yellow solid. Mass [m/z]=304.0 [M+H]⁺; ¹H-NMR (400 MHz, DMSO-d₆): 7.48 (s, 1H), 5.75 (s, 2H), 3.89 (t, J=10.8 Hz, 4H), 3.75 (t, J=4.8 Hz, 4H).

### Step 2: 4-(2-Chloro-6-((4-(methylsulfonyl)piperazin-1-yl)methyl)thieno[3,2-d]pyrimidin-4-yl)morpholine

To a stirred solution of 4-(2-chloro-6-(chloromethyl)thieno[3,2-d]pyrimidin-4-yl)morpholine (490 mg, 1.67 mmol) in IPA (5 mL) is added 1-(methylsulfonyl)piperazine (410 mg, 2.50 mmol, 1.5 eq) and DIPEA (0.6 mL, 3 eq) at room temperature. The reaction mixture is stirred at 80° C. for 12 h, monitoring the reaction by TLC. After completion, the reaction is diluted with EtOAc (30 mL) and washed with water (10 mL). Finally, the organic layer is dried over anhydrous Na₂SO₄ and evaporated under reduced pressure to obtain a crude solid which is purified by column chromatography to afford the title compound (520 mg; 82%) as an off white solid. Mass [m/z]=432 [M+H]⁺; ¹H-NMR (400 MHz, DMSO-d₆): 8.71 (br s, 1H), 7.31 (s, 1H), 3.88 (t, J=4.4 Hz, 4H), 3.91 (s, 2H), 3.74 (t, J=3.6 Hz, 4H), 3.12-3.16 (m, 6H), 2.89 (s, 3H), 2.57 (t, J=4.8 Hz, 4H).

### Step 3: 4-(6-((4-(Methylsulfonyl)piperazin-1-yl)methyl)-2-(1H-pyrazol-3-yl)thieno[3,2-d]pyrimidin-4-yl)morpholine

To a stirred solution of 4-(2-chloro-6-((4-(methylsulfonyl)piperazin-1-yl)methyl)thieno[3,2-d]pyrimidin-4-yl)morpholine (300 mg, 0.23 mmol) and pyrazole boronic acid ester (150 mg, 0.35 mmol, 1.5 eq.) in mixture of toluene and water (1:1) (4 mL) is added Pd(dppf)Cl₂ (18 mg, 0.02 mmol, 0.1 eq.), followed by K₂CO₃ (62 mg, 0.46 mmol, 2 eq.) and the mixture is degassed with nitrogen for 30 min. The reaction mixture is stirred at 110° C. for 24 h. After consumption of the starting material, the reaction mixture is filtered by passing through a celite bed. The filtrate is diluted with EtOAc (20 mL), and washed with water (10 mL). Finally, the organic layer is dried over anhydrous Na₂SO₄, filtered, and evaporated under reduced pressure to provide a brown gummy crude product which is purified by Flash chromatography (C18 reverse phase 12 g column) using 70-80% acetonitrile/water as an eluent to afford the title compound (140 mg; 42%) as an off white solid. Mass [m/z]=464.1 [M+H]⁺; ¹H-NMR (400 MHz, DMSO-d₆): 13.50 (br s, 1H), 7.58 (Br s, 1H), 7.36 (s, 1H), 6.86 (d, J=1.6 Hz, 1H), 4.00 (br s, 4H), 3.92 (s, 2H), 3.77 (t, J=4.4 Hz, 4H), 3.15 (br s, 4H), 2.89 (s, 3H), 2.67 (t, J=4.8 Hz, 4H), 2.07 (s, 1H).

### Step 4: 4-(2-(1-(3-Chlorophenyl)-1H-pyrazol-3-yl)-6-((4-(methylsulfonyl)piperazin-1-yl)methyl)thieno[3,2-d]pyrimidin-4-yl)morpholine

To a stirred solution of 4-(6-((4-(methylsulfonyl)piperazin-1-yl)methyl)-2-(1H-pyrazol-3-yl)thieno[3,2-d]pyrimidin-4-yl)morpholine (30 mg, 0.065 mmol, 1 eq.) in DMF (2 mL), is added m-chlorobenzeneboronic acid (19 mg, 0.065 mmol, 1 eq), pyridine (11 mg, 0.13 mmol, 2 eq) and Cu(OAc)₂ (17 mg, 0.095 mmol, 1.5 eq) at RT, and then the reaction temperature is raised to 90° C. and the reaction is stirred for 16 h. The reaction mixture is diluted with DCM, washed with water, and the organic layer is dried over Na₂SO₄, filtered, and evaporated under reduced pressure to obtain a crude product which is purified by preparative HPLC using acetonitrile and water as eluants to afford the title compound (3.5 mg; 10%) as an off-white solid. Mass [m/z]=574 [M+H]⁺; ¹H-NMR (400 MHz, DMSO-d₆): 8.67 (d, J=2.4 Hz, 1H), 8.05 (t, J=2 Hz, 1H), 7.93-7.96 (dd, J=1.6 Hz, 2.8 Hz, 1H), 7.75 (t, J=4 Hz, 1H), 7.44 (s, 1H), 7.4-7.42 (dd, J=1.2 Hz, 5.6 Hz, 1H), 7.18 (d, J=2.8 Hz, 1H), 4.19 (m, 4H), 4.16 (s, 2H), 3.16 (s, 4H), 2.98 (s, 3H), 2.60 (m, 4H), 1.23 (br s, 2H).

### Examples 175 and 176

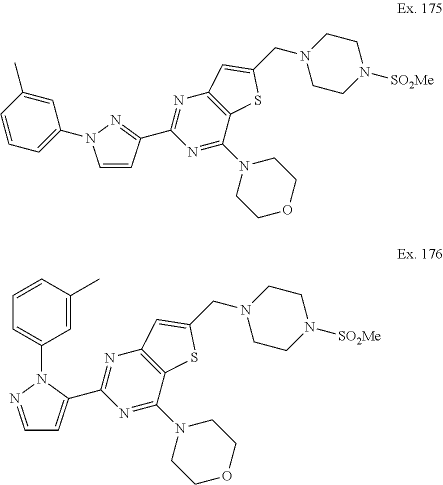 [SMILES: Cc1cccc(-n2ccc(-c3nc(N4CCOCC4)c4sc(CN5CCN(S(C)(=O)=O)CC5)cc4n3)n2)c1 ; Cc1cccc(-n2nccc2-c2nc(N3CCOCC3)c3sc(CN4CCN(S(C)(=O)=O)CC4)cc3n2)c1]

### Step 4

Following the procedure according to Example 174, 4-(6-((4-(methylsulfonyl)piperazin-1-yl)methyl)-2-(1H-pyrazol-3-yl)thieno[3,2-d]pyrimidin-4-yl)morpholine (50 mg, 0.107 mmol), m-tolyl boronic acid (19 mg, 0.017, 1 eq), pyridine (17 mg, 0.21 mmol, 2 eq), and Cu(OAc)₂ (29 mg, 0.16 mmol, 1.5 eq) are reacted at RT in DMF (2 mL), and purified by preparative HPLC to afford the compound of Example 175 (1.8 mg) and the compound of Example 176 (6 mg) as white solids. Ex. 175: Mass [m/z]=554.1 [M+H]⁺; ¹H-NMR (400 MHz, DMSO-d₆): 8.54 (d, J=2 Hz, 1H), 7.78 (s, 1H), 7.71 (d, J=8 Hz, 1H), 7.43 (s, 1H), 7.41 (t, J=8 Hz, 1H), 7.15-7.18 (m, 1H), 3.97 (m, 4H), 3.93 (s, 2H), 3.79 (br s, 4H), 3.15 (br s, 4H), 2.90 (s, 3H), 2.60 (br s, 4H), 2.42 (s, 3H). Ex. 176: Mass [m/z]=554.1 [M+H]⁺; ¹H-NMR (400 MHz, DMSO-d₆): 7.72 (d, J=2 Hz, 1H), 7.35 (s, 1H), 7.30 (t, J=7.6 Hz, 1H), 7.19-7.22 (m, 2H), 7.04 (d, J=8 Hz, 1H), 7.02 (d, J=1.6 Hz, 1H), 3.91 (s, 2H), 3.47 (t, J=7.2 Hz, 4H), 3.33 (t, J=4 Hz, 4H), 3.13 (br s, 4H), 2.89 (s, 3H), 2.57 (br s, 4H), 2.33 (s, 3H).

### Example 177

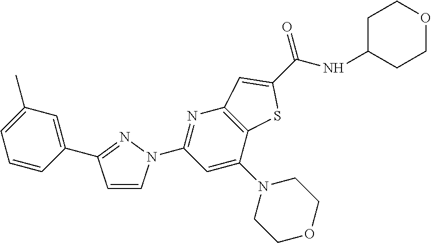 [SMILES: Cc1cccc(-c2ccn(-c3cc(N4CCOCC4)c4sc(C(=O)NC5CCOCC5)cc4n3)n2)c1]

### Step 1: 5-Chloro-7-morpholinothieno[3,2-b]pyridine-2-carboxylic acid

To a stirred solution of 4-(5-chlorothieno[3,2-b]pyridin-7-yl)morpholine (30 mg, 0.118 mmol) in dry THE (5 mL) at −78° C. is added n-BuLi (2.5M) (0.07 mL, 0.177 mmol, 2 eq) and the reaction is stirred for 1 h, and then dry ice is added. The mixture is stirred for 1 h at 0° C., quenched with saturated aqueous NH₄Cl (5 mL) and extracted with EtOAc (2×20 mL). The organic layer is dried over anhydrous Na₂SO₄, filtered, and evaporated under reduced pressure to afford the title compound (20 mg) as an off-white solid. Mass [m/z]=299.0 [M+H]⁺; ¹H-NMR (400 MHz, DMSO-d₆): 7.32 (s, 1H), 6.73 (s, 1H), 3.81 (t, J=4.4 Hz, 4H), 3.48 (t, J=4.8 Hz, 4H).

### Step 2: 5-Chloro-7-morpholino-N-(tetrahydro-2H-pyran-4-yl)thieno[3,2-b]pyridine-2-carboxamide

To a stirred solution of 5-chloro-7-morpholinothieno[3,2-b]pyridine-2-carboxylic acid (50 mg, 1 eq.), is added tetrahydro-2H-pyran-4-amine (25 mg, 1.5 eq.) in DCM (10 vol) followed by propylphosphonic anhydride (1.5 eq.) and triethylamine (2.0 eq.). The reaction is stirred at room temperature for 18 h, then diluted with ethyl acetate (20 vol) and the organic layer is washed with ice cold water (2×15 vol), brine solution (20 vol) and finally dried over Na₂SO₄, filtered, and evaporated under vacuum to provide a crude product, which is purified by silica gel chromatography (60-120 mesh) eluting with 40-80% EtOAc/hexane to afford the title compound as an off-white solid. Mass [m/z]=382 [M+H]⁺; ¹H-NMR (400 MHz, DMSO-d₆): 8.75 (d, J=2.8 Hz, 1H), 8.11 (s, 1H), 6.87 (s, 1H), 4.07-3.96 (m, 1H), 3.90-3.88 (d, J=9.6 Hz, 2H), 3.80 (t, J=4.4 Hz, 4H), 3.50 (t, J=4.4 Hz, 4H), 342-3.36 (m, 2H), 1.80-1.78 (m, 2H), 1.64-1.54 (m, 2H).

### Step 3: 7-Morpholino-N-(tetrahydro-2H-pyran-4-yl)-5-(3-(m-tolyl)-1H-pyrazol-1-yl)thieno[3,2-b]pyridine-2-carboxamide

To a solution of compound 5-chloro-7-morpholino-N-(tetrahydro-2H-pyran-4-yl)thieno[3,2-b]pyridine-2-carboxamide (40 mg, 1 eq) in 1,4 dioxane and toluene (1:1, 20 vol) is added 3-(m-tolyl)-1H-pyrazole (16 mg, 1.0 eq) and K₃PO₄ (2 eq). The reaction mixture is degassed for 15 min under nitrogen followed by the addition of Pd₂(dba)₃ (0.1 eq) and t-Bu-XPhos (0.2 eq) at room temperature. The reaction temperature is raised to 150° C. and the mixture is stirred for 16 h. The progress of the reaction is monitored by TLC. After completion, the reaction mixture is cooled to room temperature, and it is filtered through a celite pad, and the pad is washed with ethyl acetate (100 vol). The filtrate is concentrated under reduced pressure to provide a crude solid which is purified by recrystallization, flash reverse phase chromatography, or preparative HPLC to afford the title compound (4.5 mg; 8%) as an off white solid. Mass [m/z]=504.2 [M+H]⁺; ¹H-NMR (400 MHz, DMSO-d₆): 8.73 (d, J=8 Hz, 1H), 8.69 (d, J=2.4 Hz, 1H), 8.24 (s, 1H), 7.80 (d, J=9.2 Hz, 2H), 7.52 (s, 1H), 7.39 (t, J=7.6 Hz, 1H), 7.22 (d, J=7.2, 1H), 7.09 (d, J=2.4 Hz, 1H), 4.07-3.99 (m, 1H), 3.91 (d, J=9.6 Hz, 2H), 3.87 (t, J=4.4 Hz, 4H), 3.56 (t, J=4.4 Hz, 4H), 343-3.37 (m, 2H), 2.40 (s, 1H), 1.83-1.79 (m, 2H), 1.65-1.55 (m, 2H).

### Example 178

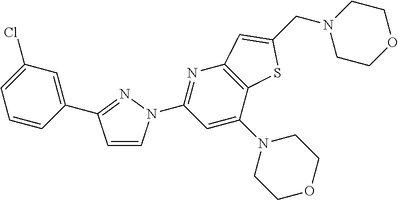 [SMILES: Clc1cccc(-c2ccn(-c3cc(N4CCOCC4)c4sc(CN5CCOCC5)cc4n3)n2)c1]

### Step 1: 4-(5-(3-(3-chlorophenyl)-1H-pyrazol-1-yl)-2-(morpholinomethyl)thieno[3,2-b]pyridin-7-yl)morpholine

To a stirred solution of 4-(5-(3-bromo-1H-pyrazol-1-yl)-2-(morpholinomethyl)thieno[3,2-b]pyridin-7-yl)morpholine (50 mg, 1.0 equiv.) in toluene and EtOH (1:1, 10 vol) is added (3-chlorophenyl)boronic acid (2.0 equiv.), followed by K₂CO₃ (2.0 equiv.) at room temperature. The mixture is degassed with nitrogen for 15 min then Pd(dppf)Cl₂·DCM complex (0.2 eq) is added and again the mixture is degassed for 15 min. The reaction is heated to 120° C. and stirred for 16 h after which water (10 vol) is added and the mixture is extracted with EtOAc (2×10 vol). The combined organic layers are dried over anhydrous Na₂SO₄, filtered and evaporated under reduced pressure to provide a crude solid which is purified by silica gel chromatography or preparative HPLC to afford the title compound (18 mg, 33%) as off-white solid. Mass [m/z]=496 [M+H]⁺; ¹H-NMR (400 MHz, DMSO-d₆): 8.70 (d, J=2.8 Hz, 1H), 8.03 (s, 1H), 7.97 (d, J=7.6 Hz, 1H), 7.44-7.53 (m, 3H), 7.38 (s, 1H), 7.16 (d, J=2.4 Hz, 1H), 3.83-3.86 (m, 6H), 3.62 (t, J=4.0 Hz, 4H), 3.52 (t, J=4.4 Hz, 4H), 2.46 (br s, 4H).

### Example 179

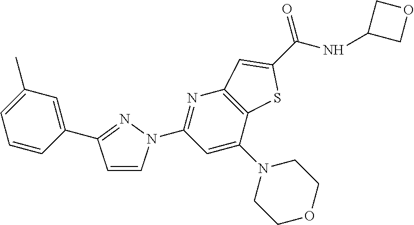 [SMILES: Cc1cccc(-c2ccn(-c3cc(N4CCOCC4)c4sc(C(=O)NC5COC5)cc4n3)n2)c1]

### Step 2: 5-Chloro-7-morpholino-N-(oxetan-3-yl)thieno[3,2-b]pyridine-2-carboxamide

Following the procedure according to Example 177, 5-chloro-7-morpholinothieno[3,2-b]pyridine-2-carboxylic acid (50 mg) is treated with oxetan-3-amine (18 mg) to afford the title compound (40 mg; 85%) as an off-white solid. Mass [m/z]=354.1 [M+H]⁺; ¹H-NMR (400 MHz, DMSO-d₆): 9.55 (d, J=6.4 Hz, 1H), 8.15 (s, 1H), 6.88 (s, 1H), 5.05-5.00 (m, 1H), 4.82 (t, J=6.4 Hz, 2H), 4.63 (t, J=6.4 Hz, 2H), 3.80 (t, J=4.4 Hz, 4H), 3.50 (t, J=4.8 Hz, 4H).

### Step 3: 7-Morpholino-N-(oxetan-3-yl)-5-(3-(m-tolyl)-1H-pyrazol-1-yl)thieno[3,2-b]pyridine-2-carboxamide

Following the procedure according to Example 177, 5-chloro-7-morpholino-N-(oxetan-3-yl)thieno[3,2-b]pyridine-2-carboxamide (40 mg) and 3-(m-tolyl)-1H-pyrazole (18 mg) are reacted to afford the title compound (15 mg) as an off white solid. Mass [m/z]=474.1 [M+H]⁺; ¹H-NMR (400 MHz, DMSO-d₆): 9.53 (d, J=6.4 Hz, 1H), 8.71 (d, J=2.8 Hz, 1H), 8.27 (s, 1H), 7.81-7.78 (m, 2H), 7.53 (s, 1H), 7.50-7.46 (m, 1H), 7.39-7.34 (m, 1H), 7.22 (d, J=7.2, 1H), 7.09 (d, J=2.4 Hz, 1H), 5.05-5.03 (m, 1H), 4.83 (t, J=6.4 Hz, 2H), 4.69 (t, J=6.4 Hz, 2H), 3.87 (t, J=4.4 Hz, 4H), 3.56 (t, J=4.8 Hz, 4H) 2.40 (s, 3H).

### Example 181

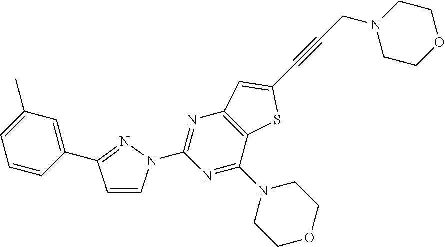 [SMILES: Cc1cccc(-c2ccn(-c3nc(N4CCOCC4)c4sc(C#CCN5CCOCC5)cc4n3)n2)c1]

### Step 1: 3-(2-Chloro-4-morpholinothieno[3,2-d]pyrimidin-6-yl)prop-2-yn-1-ol

To a stirred solution of 4-(2-chloro-6-iodothieno[3,2-d]pyrimidin-4-yl)morpholine (500 mg, 1.315 mmol) in THE (10 mL) is added prop-2-yn-1-ol (88 mg, 1.578 mmol, 1.2 eq), CuI (13 mg, 0.065 mmol, 0.05 eq), triethylamine (8.5 mL, 59.21 mmol, 45 eq) and Pd(dppf)Cl₂ (46 mg, 0.065 mmol, 0.05 eq) at room temperature. The reaction mixture is stirred at the same temperature for 8 h and then it is diluted with EtOAc (30 mL). The organics are washed with water (20 mL), dried over anhydrous Na₂SO₄, filtered and evaporated under reduced pressure to provide a brownish gum. The crude compound is purified by silica gel chromatography to afford the title compound (320 mg; 78%) as an off white solid. Mass [m/z]=310.1 [M+H]⁺; ¹H-NMR (400 MHz, DMSO-d₆: 7.58 (s, 1H), 5.56 (t, J=6 Hz, 1H), 4.40 (d, J=6 Hz, 2H), 3.88 (t, J=4.4 Hz, 4H), 3.75 (t, J=5.2 Hz, 4H).

### Step 2: 4-(2-Chloro-6-(3-chloroprop-1-yn-1-yl)thieno[3,2-d]pyrimidin-4-yl)morpholine

To a stirred solution of 3-(2-chloro-4-morpholinothieno[3,2-d]pyrimidin-6-yl)prop-2-yn-1-ol (60 mg, 0.29 mmol) in DCM (3 mL) at 0° C. is added SOCl₂ (0.05 mL) and the reaction is warmed to room temperature and stirred for 4 h. The mixture is evaporated under reduced pressure and the resulting solid triturated with diethyl ether (5 mL), to afford the title compound (45 mg; 66%) as an off-white solid. Mass [m/z]=328.0 [M+H]⁺; ¹H-NMR (400 MHz, DMSO-d₆: 7.69 (s, 1H), 4.83 (s, 2H), 3.90 (t, J=7.2 Hz, 4H), 3.90 (t, J=5.2 Hz, 4H).

### Step 3: 4-(3-(2-Chloro-4-morpholinothieno[3,2-d]pyrimidin-6-yl)prop-2-yn-1-yl)morpholine

To a stirred solution of 4-(2-chloro-6-(3-chloroprop-1-yn-1-yl)thieno[3,2-d]pyrimidin-4-yl)morpholine (45 mg, 1.0 equiv.) in IPA (10 vol) at room temperature is added morpholine (18 mg, 1.5 equiv.) followed by DIPEA (3 equiv.). The reaction mixture is stirred at 80° C. for 12 h, then it is diluted with water (10 vol) and extracted with DCM (2×20 vol). The combined organic layers are dried over anhydrous Na₂SO₄, filtered, and evaporated under reduced pressure to provide a crude compound which is purified by reverse phase flash column chromatography or by trituration with diethyl ether to afford the title compound (40 mg; 76%) as an off-white solid. Mass [m/z]=379.1 [M+H]⁺; ¹H-NMR (400 MHz, CDCl₃): 7.37 (s, 1H), 3.98 (t, J=4.4 Hz, 4H), 3.85 (t, J=5.2 Hz, 4H), 3.79 (t, J=4.4 Hz, 4H), 3.59 (s, 2H), 2.66 (t, J=4.4 Hz, 4H).

### Step 4

To a solution of 4-(3-(2-chloro-4-morpholinothieno[3,2-d]pyrimidin-6-yl)prop-2-yn-1-yl)morpholine (40 mg, 1 eq) in a mixture of 1,4-dioxane: toluene (1:1, 20 vol) is added 3-(m-tolyl)-1H-pyrazole (25 mg, 1.5 eq) and K₃PO₄ (2 eq). The reaction mixture is degassed with nitrogen for 15 min followed by the addition of Pd₂(dba)₃ (0.1 eq) and t-Bu-XPhos (0.2 eq.) at room temperature. The reaction mixture is heated to 150° C. and stirred for 16 h. The progress of the reaction is monitored by TLC, and after completion the reaction mixture is cooled to room temperature and filtered through a celite pad, which is washed with ethyl acetate (100 vol). The filtrate is concentrated under reduced pressure and the resulting crude solid is purified by preparative HPLC to afford the title compound (4 mg; 8%) as an off-white solid. Mass [m/z]=501.3 [M+H]⁺; ¹H-NMR (400 MHz, DMSO-d₆): 8.74 (d, J=2.8 Hz, 1H), 7.79 (br s, 2H), 7.57 (d, J=8 Hz, 1H), 7.71 (s, 1H), 7.37 (t, J=7.6, 1H), 7.21 (d, J=7.2, 1H), 7.04 (d, J=2.4 Hz, 1H), 4.01 (t, J=4.4 Hz, 4H), 3.81 (t, J=4.4 Hz, 8H), 3.67 (s, 2H), 3.63 (t, J=4.4 Hz, 4H), 2.54 (t, J=4.4 Hz, 4H), 2.39 (s, 3H).

### Example 182

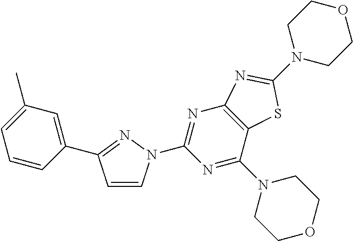 [SMILES: Cc1cccc(-c2ccn(-c3nc(N4CCOCC4)c4sc(N5CCOCC5)nc4n3)n2)c1]

### Step 1: Potassium cyanocarbamodithioate

A solution of cyanamide (5 g, 119.04 mmol) and carbon disulfide (9.9 g, 130.9 mmol, 1.1 eq) is stirred at room temperature for 30 min, then cooled to 0° C., followed by the dropwise addition of KOH (13.3 g, 238.09 mmol, 2 eq) in 95% EtOH (50 mL). After the final addition, the reaction mixture is stirred at room temperature for 15 h, filtered and the wet cake is washed with EtOH (10 mL) to afford the title compound (10.2 g; 57%) as an off-white solid.

### Step 2: Methyl cyanocarbamodithioate

To a solution of potassium cyanocarbamodithioate (10 g, 51.5 mmol) in acetone (40 mL) and water (45 mL) is added dropwise Mel (7.31 g, 51.5 mmol, 1 eq) at 0° C. After completion of the addition, the resulting mixture is stirred at room temperature for 2 h and then it is concentrated in vacuo. Acetone (100 mL) is added to the residue and the mixture stirred at room temperature for 30 min, filtered and the filtrate is concentrated in vacuo and the resulting residue is stirred with MTBE (15 mL). The resulting suspension is filtered and dried to provide the title compound (5.5 g; 66%) as an off white solid. Mass [m/z]=131 [M−H]⁺; ¹H-NMR (400 MHz, DMSO-d₆): 2.25 (s, 3H).

### Step 3: Ethyl 4-amino-2-(methylthio)thiazole-5-carboxylate

A solution of methyl cyanocarbamodithioate (15 g, 113.63 mmol) and ethyl bromoacetate (13 mL, 79.54 mmol, 1 eq) in EtOH (200 mL) is heated at reflux for 1 h, cooled to room temperature and triethylamine (24 mL, 170.45 mmol, 1.5 eq) after which the reaction mixture is heated at reflux for an additional 5 h. The reaction solvent is evaporated in vacuo and water added to form a precipitate, which is filtered and dried to provide a brown solid which is purified by trituration with diethyl ether to afford the title compound (8.2 g; 24%) as a brown solid. Mass [m/z]=219.1 [M−H]⁺; ¹H-NMR (400 MHz, DMSO-d₆): 7.01 (br s, 2H), 4.19-4.14 (q, J=14 Hz, 2H), 2.65 (s, 3H), 1.24 (t, J=7.2 Hz, J=6.8 Hz, 3H).

### Step 4: Ethyl 2-(methylthio)-4-ureidothiazole-5-carboxylate

To a solution of ethyl 4-amino-2-(methylthio)thiazole-5-carboxylate (5 g, 22.93 mmol) in dry DCM (30 ml) is added chlorosulfonyl isocyanate (1.5 mL, 32.11 mmol, 1.4 eq) at −78° C., and the solution is stirred for 10 min. The reaction is allowed to warm to 0° C., stirred for 2 h and then quenched with 6N aqueous HCl (10 mL). The reaction mixture is then heated to 100° C. and maintained for 3 h, then cooled to room temperature and quenched with saturated aqueous sodium carbonate solution (50 mL) to yield a solid which is filtered and dried to afford the title compound (4.8 g; 80%) as a brown solid. Mass [m/z]=262.1 [M−H]⁺; ¹H-NMR (400 MHz, DMSO-d₆): 8.67 (s, 1H), 7.37 (br s, 2H), 4.29-4.24 (m, 2H), 2.74 (s, 3H), 1.28 (t, J=6.8, 3H).

### Step 5: 2-(Methylthio)thiazolo[4,5-d]pyrimidine-5,7 (4H,6H)-dione

A solution of ethyl 2-(methylthio)-4-ureidothiazole-5-carboxylate (2.5 g, 9.57 mmol) in 7M methanolic NH₃ (60 ml) was loaded in a steel bomb. The reaction mixture is heated at 80° C. for 16 h. The reaction mixture is cooled to room temperature, the solvent is evaporated and the crude solid is triturated with diethyl ether to afford the title compound (1.9 g, 95%) as an off-white solid. Mass [m/z]=216.0 [M−H]⁺; ¹H-NMR (400 MHz, DMSO-d₆): 8.01 (br s, 2H), 2.72 (s, 3H).

### Step 6: 5,7-Dichloro-2-(methylthio)thiazolo[4,5-d]pyrimidine

A solution of 2-(methylthio)thiazolo[4,5-d]pyrimidine-5,7-(4H,6H)-dione (2 g, 9.30 mmol) in neat POCl₃ (20 mL) is heated at 100° C. The progress of the reaction is monitored by TLC. Upon complete consumption of the starting material, the reaction is cooled to room temperature and the solvent is evaporated to provide a brown gummy solid. Ice-cold water is added to the gummy solid and it is stirred for 15 min to provide a precipitate which is filtered and dried to afford the title compound (1.6 g; 69%) as an off white solid. Mass [m/z]=251.9 [M−H]⁺; ¹H-NMR (400 MHz, CDCl₃): 2.89 (s, 3H).

### Step 7: 4-(5-Chloro-2-(methylthio)thiazolo[4,5-d]pyrimidin-7-yl)morpholine

To a solution of 5,7-dichloro-2-(methylthio)thiazolo[4,5-d]pyrimidine (1.5 g, 5.97 mmol) in MeOH (10 mL) is added morpholine (0.52 mL, 5.97 mmol, 1 eq) at RT and the mixture is stirred for 8 h. After completion, the solid is filtered, triturated with MeOH (5 mL) and dried under high vacuum to afford the title compound (1.4 g; 77%) as an off white solid. Mass [m/z]=303.0 [M−H]⁺; ¹H-NMR (400 MHz, DMSO-d₆): 3.79 (t, J=4.4 Hz, 4H), 3.74 (t, J=4.4 Hz, 4H), 2.81 (s, 3H).

### Step 8: 4-(5-Chloro-2-(methylsulfonyl)thiazolo[4,5-d]pyrimidin-7-yl)morpholine

To a solution of 4-(5-chloro-2-(methylthio)thiazolo[4,5-d]pyrimidin-7-yl)morpholine (500 mg, 1.65 mmol) in MeOH/water (9:1, 12 mL) is added Oxone (2.5 g, 4.13 mmol, 2.5 eq) at RT and the reaction is heated to 60° C. and stirred for 5 h. The reaction mixture is filtered and the solid is washed with DCM (20 mL). The filtrate is washed with saturated aqueous sodium bicarbonate, dried over Na₂SO₄, and concentrated to provide a solid which is triturated with diethyl ether to afford the title compound (380 mg, 69%) as an off-white solid. Mass [m/z]=335.1 [M−H]⁺; ¹H-NMR (400 MHz, DMSO-d₆): 3.93 (t, J=4.4 Hz, 4H), 3.78 (t, J=4.4 Hz, 4H), 3.63 (s, 3H).

### Step 9: 4,4′-(5-Chlorothiazolo[4,5-d]pyrimidine-2,7-diyl)dimorpholine

To a stirred solution of 4-(5-chloro-2-(methylsulfonyl)thiazolo[4,5-d]pyrimidin-7-yl)morpholine (100 mg, 1.0 equiv.) in THE (10 vol) at room temperature is added morpholine (36 mg, 1.4 equiv.) followed by triethylamine (1.4 equiv.) and the mixture is stirred for 2 h. The progress of the reaction is monitored by TLC. After completion, the reaction mixture is diluted with water (10 vol) and extracted with DCM (2×20 vol). The combined organic layers are dried over anhydrous Na₂SO₄, filtered, and evaporated under reduced pressure to provide a crude product which is purified by RP flash column chromatography or by trituration with diethyl ether to afford the title compound (80 mg; 78%) as an off-white solid. Mass [m/z]=342.2 [M+H]⁺; ¹H-NMR (400 MHz, DMSO-d₆): 3.83 (t, J=4.8 Hz, J=7.2 Hz, 12H), 3.71 (t, J=4.4 Hz, 4H).

### Step 10: 4,4′-(5-(3-(m-Tolyl)-1H-pyrazol-1-yl)thiazolo[4,5-d]pyrimidine-2,7-diyl)dimorpholine

To a solution of 4,4′-(5-chlorothiazolo[4,5-d]pyrimidine-2,7-diyl)dimorpholine (40 mg, 1 eq) in a mixture of 1,4-dioxane and toluene (1:1, 20 vol) is added 3-(m-tolyl)-1H-pyrazole (20 mg, 1 eq) and K₃PO₄ (2 eq) at room temperature and the mixture is degassed with nitrogen for 15 min. Then Pd₂(dba)₃ (0.1 eq) and t-Bu-XPhos (0.2 eq) are added and the mixture is heated to 140° C. and stirred for 16 h. The reaction mixture is cooled to room temperature, filtered through a celite pad, washed with ethyl acetate (100 vol) and the filtrate is concentrated under reduced pressure to provide a crude product residue which is purified by preparative HPLC to afford the title compound (13 mg; 24%) as an off-white solid. Mass [m/z]=464.2 [M+H]⁺; ¹H-NMR (400 MHz, DMSO-d₆): 8.68 (d, J=2.8 Hz, 1H), 7.79 (br s, 2H), 7.51 (d, J=8 Hz, 1H), 7.36 (t, J=7.6, 1H), 7.20 (d, J=7.2, 1H), 7.01 (d, J=2.4 Hz, 1H), 3.85 (t, J=4.4 Hz, 4H), 3.77 (t, J=4.4 Hz, 8H), 3.67 (t, J=4.4 Hz, 4H), 2.39 (s, 3H).

### Example 183

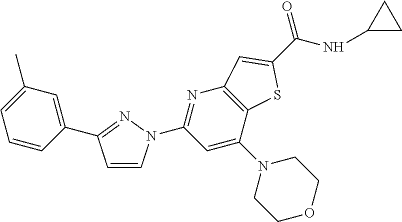 [SMILES: Cc1cccc(-c2ccn(-c3cc(N4CCOCC4)c4sc(C(=O)NC5CC5)cc4n3)n2)c1]

### Step 2: 5-Chloro-N-cyclopropyl-7-morpholinothieno[3,2-b]pyridine-2-carboxamide

Following the procedure according to Example 177, 5-chloro-7-morpholinothieno[3,2-b]pyridine-2-carboxylic acid (60 mg) is treated with cyclopropanamine (17 mg) to afford the title compound (40 mg; 60%) as an off-white solid. Mass [m/z]=338.1 [M+H]⁺; ¹H-NMR (400 MHz, DMSO-d₆): 8.89 (d, J=4 Hz, 1H), 8.01 (s, 1H), 6.88 (s, 1H), 6.86 (s, 1H), 3.80 (t, J=4.4 Hz, 4H), 3.50 (t, J=4.8 Hz, 4H), 0.77-0.72 (m, 2H), 0.6-0.61 (m, 2H).

### Step 3: N-cyclopropyl-7-morpholino-5-(3-(m-tolyl)-1H-pyrazol-1-yl)thieno[3,2-b]pyridine-2-carboxamide

Following the procedure according to Example 177, 5-chloro-N-cyclopropyl-7-morpholinothieno[3,2-b]pyridine-2-carboxamide (40 mg) and 3-(m-tolyl)-1H-pyrazole (18 mg) were reacted to afford the title compound (14 mg; 26%) as an off-white solid. Mass [m/z]=460.2 [M+H]⁺; ¹H-NMR (400 MHz, DMSO-d₆): 8.86 (d, J=4 Hz, 1H), 8.68 (d, J=2.4 Hz, 1H), 8.13 (s, 1H), 7.80 (d, J=9.2 Hz, 2H), 7.51 (s, 1H), 7.38 (t, J=7.6 Hz, 1H), 7.22 (d, J=7.2, 1H), 7.08 (d, J=2.4 Hz, 1H), 3.87 (t, J=4.4 Hz, 4H), 3.56 (t, J=4.8 Hz, 4H), 2.91-2.84 (m, 1H), 0.78-0.73 (m, 2H), 0.64-0.62 (m, 2H).

### Example 184

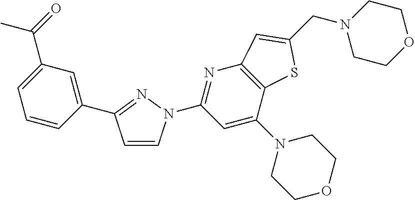 [SMILES: CC(=O)c1cccc(-c2ccn(-c3cc(N4CCOCC4)c4sc(CN5CCOCC5)cc4n3)n2)c1]

### 1-(3-(1-(7-Morpholino-2-(morpholinomethyl)thieno[3,2-b]pyridin-5-yl)-1H-pyrazol-3-yl)phenyl)ethan-1-one

Following the procedure according to Example 178, 4-(5-(3-bromo-1H-pyrazol-1-yl)-2-(morpholinomethyl)thieno[3,2-b]pyridin-7-yl)morpholine (80 mg) is reacted with (3-acetylphenyl)boronic acid to afford the title compound (25 mg, 38%) as a grey solid.

Mass [m/z]=504 [M+H]⁺; ¹H-NMR (400 MHz, DMSO-d₆): 8.72 (d, J=2.4 Hz, 1H), 8.46 (s, 1H), 8.27 (d, J=8.0 Hz, 1H), 7.99 (d, J=8.0 Hz, 1H), 7.66 (t, J=7.6 Hz, 1H), 7.44 (s, 1H), 7.39 (s, 1H), 7.20 (d, J=2.8 Hz, 1H), 3.84-3.86 (m, 6H), 3.62 (t, J=4.4 Hz, 4H), 3.53 (t, J=4.8 Hz, 4H), 2.67 (s, 3H), 2.49-2.50 (m, 4H).

### Example 185

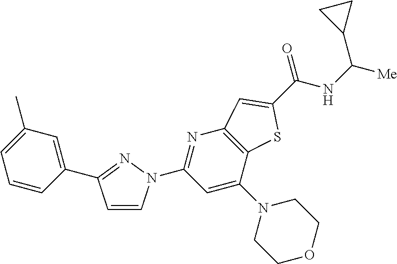 [SMILES: Cc1cccc(-c2ccn(-c3cc(N4CCOCC4)c4sc(C(=O)NC(C)C5CC5)cc4n3)n2)c1]

### Step 2: 5-Chloro-N-(1-cyclopropylethyl)-7-morpholinothieno[3,2-b]pyridine-2-carboxamide

Following the procedure according to Example 177, 5-chloro-7-morpholinothieno[3,2-b]pyridine-2-carboxylic acid (80 mg) is treated with 1-cyclopropylethan-1-amine (17 mg) to afford the title compound (50 mg; 58%) as an off-white solid. Mass [m/z]=381.1 [M+H]⁺; ¹H-NMR (400 MHz, DMSO-d₆): 9.33 (d, J=4 Hz, 1H), 8.23 (s, 1H), 6.89 (s, 1H), 4.60 (br s, 1H), 3.80 (t, J=4.4 Hz, 4H), 3.50 (t, J=4.8 Hz, 4H), 3.10-3.05 (m, 2H), 2.82 (s, 3H), 1.51 (br s, 1H), 1.23-1.14 (m, 2H).

### Step 3: N-(1-Cyclopropylethyl)-7-morpholino-5-(3-(m-tolyl)-1H-pyrazol-1-yl)thieno[3,2-b]pyridine-2-carboxamide

Following the procedure according to Example 177, 5-chloro-N-(1-cyclopropylethyl)-7-morpholinothieno[3,2-b]pyridine-2-carboxamide (50 mg) and 19 mg of 3-(m-tolyl)-1H-pyrazole (19 mg) are reacted to afford the title compound (14 mg; 22%) as an off white solid. Mass [m/z]=488.3 [M+H]⁺; ¹H-NMR (400 MHz, DMSO-d₆): 8.76 (d, J=4 Hz, 1H), 8.69 (d, J=2.4 Hz, 1H), 8.24 (s, 1H), 7.80 (d, J=9.2 Hz, 2H), 7.52 (s, 1H), 7.39 (t, J=7.6 Hz, 1H), 7.22 (d, J=7.2, 1H), 7.09 (d, J=2.4 Hz, 1H), 3.87 (t, J=4.4 Hz, 4H), 3.56 (t, J=4.8 Hz, 4H), 3.50-3.44 (m, 1H), 2.40 (s, 3H), 1.27 (d, J=6.8 Hz, 4H), 1.27-0.98 (m, 1H), 0.51-0.20 (m, 4H).

### Example 186

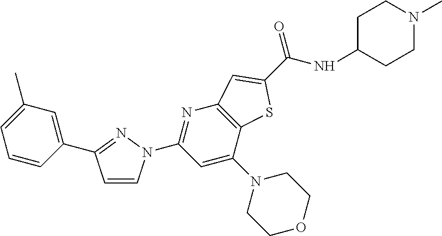 [SMILES: Cc1cccc(-c2ccn(-c3cc(N4CCOCC4)c4sc(C(=O)NC5CCN(C)CC5)cc4n3)n2)c1]

### Step 2: 5-Chloro-N-(1-methylpiperidin-4-yl)-7-morpholinothieno[3,2-b]pyridine-2-carboxamide

Following the procedure according to Example 177, 5-chloro-7-morpholinothieno[3,2-b]pyridine-2-carboxylic acid (80 mg) is treated with 1-methylpiperidin-4-amine (45 mg) to afford the title compound (55 mg; 52%) as an off-white solid. Mass [m/z]=395.1 [M+H]⁺; ¹H-NMR (400 MHz, CDCl₃): 7.7 (br s, 1H), 6.67 (s, 1H), 4.05-4.0 (br s, 1H), 3.92 (t, J=4.4 Hz, 4H), 3.48 (t, J=4.8 Hz, 4H), 2.89 (t, J=9.6 Hz, 2H), 2.34 (s, 3H), 2.23-2.17 (m, 2H), 2.08 (d, J=10 Hz, 2H).

### Step 3: N-(1-Methylpiperidin-4-yl)-7-morpholino-5-(3-(m-tolyl)-1H-pyrazol-1-yl)thieno[3,2-b]pyridine-2-carboxamide

Following the procedure according to Example 177, 5-chloro-N-(1-methylpiperidin-4-yl)-7-morpholinothieno[3,2-b]pyridine-2-carboxamide (55 mg) and 3-(m-tolyl)-1H-pyrazole (19 mg) are reacted to afford the title compound (16 mg; 22%) as an off white solid. Mass [m/z]=517.5 [M+H]⁺; ¹H-NMR (400 MHz, DMSO-d₆): 8.68-8.65 (m, 2H), 8.24 (s, 1H), 7.80 (d, J=9.2 Hz, 2H), 7.52 (s, 1H), 7.38 (t, J=7.6 Hz, 1H), 7.22 (d, J=7.2, 1H), 7.08 (d, J=2.4 Hz, 1H), 3.87 (t, J=4.4 Hz, 4H), 3.75-3.72 (m, 1H), 3.56 (t, J=4.8 Hz, 4H), 2.82-2.79 (m, 2H), 2.40 (s, 3H), 2.19 (s, 3H), 2.02-1.97 (m, 2H), 1.83-1.80 (m, 2H), 1.65-1.59 (m, 2H).

### Example 187

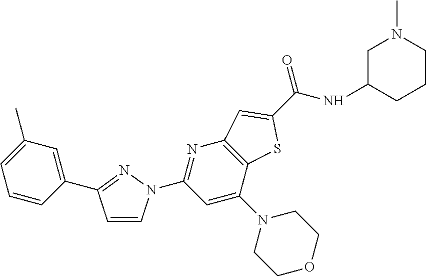 [SMILES: Cc1cccc(-c2ccn(-c3cc(N4CCOCC4)c4sc(C(=O)NC5CCCN(C)C5)cc4n3)n2)c1]

### Step 2: 5-Chloro-N-(1-methylpiperidin-3-yl)-7-morpholinothieno[3,2-b]pyridine-2-carboxamide

Following the procedure according to Example 177, 5-chloro-7-morpholinothieno[3,2-b]pyridine-2-carboxylic acid (80 mg) is treated with 1-methylpiperidin-3-amine (45 mg) to afford the title compound (70 mg; 66%) as an off-white solid. Mass [m/z]=395.2 [M+H]⁺; ¹H-NMR (400 MHz, CDCl₃): 7.81 (br s, 1H), 6.66 (s, 1H), 4.32 (br s, 1H), 3.92 (t, J=4.4 Hz, 4H), 3.49 (t, J=4.8 Hz, 4H), 2.69 (br s, 2H), 2.46 (br s, 1H), 2.30 (s, 3H), 2.19-2.17 (m, 1H), 1.81 (br s, 2H), 1.64-1.61 (m, 2H).

### Step 3: N-(1-Methylpiperidin-3-yl)-7-morpholino-5-(3-(m-tolyl)-1H-pyrazol-1-yl)thieno[3,2-b]pyridine-2-carboxamide

Following the procedure according to Example 177, 5-chloro-N-(1-methylpiperidin-3-yl)-7-morpholinothieno[3,2-b]pyridine-2-carboxamide (70 mg) and 28 mg of 3-(m-tolyl)-1H-pyrazole (28 mg) are reacted to afford the title compound (10 mg; 10%) as an off white solid. Mass [m/z]=517.0 [M+H]⁺; ¹H-NMR (400 MHz, DMSO-d₆): 8.69 (d, J=2.4 Hz, 1H), 8.2 (s, 1H), 7.80 (d, J=9.2 Hz, 2H), 7.52 (s, 1H), 7.38 (t, J=7.6 Hz, 1H), 7.22 (d, J=7.2, 1H), 7.08 (d, J=2.4 Hz, 1H), 3.97 (br s, 1H), 3.87 (t, J=4.4 Hz, 4H), 3.56 (t, J=4.8 Hz, 4H), 2.94-2.76 (m, 2H), 2.40 (s, 3H), 2.34-2.28 (m, 2H), 2.08-1.99 (m, 2H), 1.90-1.74 (m, 2H), 1.58-1.55 (m, 1H), 1.38-1.36 (m, 1H).

### Example 188

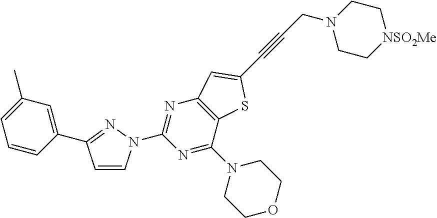

### Step 3: 4-(2-Chloro-6-(3-(4-(methylsulfonyl)piperazin-1-yl)prop-1-yn-1-yl)thieno[3,2-d]pyrimidin-4-yl)morpholine

Following the procedure according to Example 181, 4-(2-chloro-6-(3-chloroprop-1-yn-1-yl)thieno[3,2-d]pyrimidin-4-yl)morpholine (60 mg) is reacted with 1-(methylsulfonyl) piperazine (45 mg) to afford the title compound (48 mg; 65%) as an off-white solid. Mass [m/z]=456.2 [M+H]⁺; ¹H-NMR (400 MHz, CDCl₃): 7.37 (s, 1H), 3.99 (t, J=4.4 Hz, 4H), 3.85 (t, J=5.2 Hz, 4H), 3.65 (s, 2H), 3.31 (br s, 4H) 2.80 (s, 3H), 2.76 (t, J=4.4 Hz, 4H).

### Step 4: 4-(6-(3-(4-(Methylsulfonyl)piperazin-1-yl)prop-1-yn-1-yl)-2-(3-(m-tolyl)-1H-pyrazol-1-yl)thieno[3,2-d]pyrimidin-4-yl)morpholine

Following the procedure according to Example 181, 4-(2-chloro-6-(3-(4-(methylsulfonyl)piperazin-1-yl)prop-1-yn-1-yl)thieno[3,2-d]pyrimidin-4-yl)morpholine (60 mg) is reacted with 3-(m-tolyl)-1H-pyrazole (31 mg) to afford the title compound (4 mg; 5%) as an off-white solid. Mass [m/z]=578.3 [M+H]⁺; ¹H-NMR (400 MHz, DMSO-d₆): 8.74 (d, J=2.4 Hz, 1H), 7.79 (br s, 1H), 7.75-721 (m, 2H), 7.37 (t, J=7.6, 1H), 7.21 (d, J=7.2, 1H), 7.04 (d, J=2.4 Hz, 1H), 4.01 (t, J=4.4 Hz, 4H), 3.81 (t, J=4.4 Hz, 8H), 3.76 (s, 2H), 3.17 (t, J=4.4 Hz, 4H), 2.89 (s, 3H), 2.67 (t, J=4.8 Hz, 4H), 2.39 (s, 3H).

### Example 189

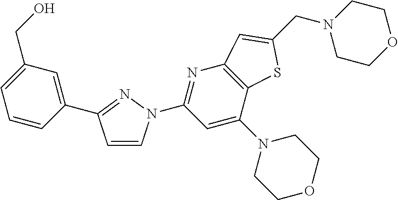 [SMILES: OCc1cccc(-c2ccn(-c3cc(N4CCOCC4)c4sc(CN5CCOCC5)cc4n3)n2)c1]

### (3-(1-(7-Morpholino-2-(morpholinomethyl)thieno[3,2-b]pyridin-5-yl)-1H-pyrazol-3-yl)phenyl)methanol

Following the procedure according to Example 178, 4-(5-(3-bromo-1H-pyrazol-1-yl)-2-(morpholinomethyl)thieno[3,2-b]pyridin-7-yl)morpholine (80 mg) is reacted with (3-(hydroxymethyl)phenyl)boronic acid to afford the title compound (4 mg, 6%) as an off white solid. Mass [m/z]=492.3 [M+H]⁺; ¹H-NMR (400 MHz, DMSO-d₆): 8.68 (d, J=2.8 Hz, 1H), 7.91 (s, 1H), 7.85 (d, J=7.6 Hz, 1H), 7.33-7.45 (m, 4H), 7.04 (d, J=2.8 Hz, 1H), 5.28 (t, J=6.0 Hz, 1H), 4.59 (d, J=5.6 Hz, 2H), 3.83-3.86 (m, 6H), 3.62 (t, J=4.0 Hz, 4H), 3.51 (t, J=4.4 Hz, 4H), 2.49-2.50 (m, 4H), 3.15 (s, 4H), 2.90 (s, 3H), 2.59 (s, 4H), 2.39 (s, 3H).

### Example 190

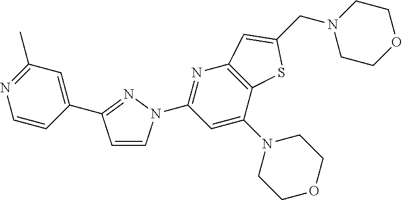 [SMILES: Cc1cc(-c2ccn(-c3cc(N4CCOCC4)c4sc(CN5CCOCC5)cc4n3)n2)ccn1]

### 4-(5-(3-(2-Methylpyridin-4-yl)-1H-pyrazol-1-yl)-2-(morpholinomethyl)thieno[3,2-b]pyridin-7-yl) morpholine

Following the procedure according to Example 178, 4-(5-(3-bromo-1H-pyrazol-1-yl)-2-(morpholinomethyl)thieno[3,2-b]pyridin-7-yl)morpholine (80 mg) is reacted with (2-methylpyridin-4-yl)boronic acid to afford the title compound (25 mg, 30%) as off white solid. Mass [m/z]=477 [M+H]⁺; ¹H-NMR (400 MHz, DMSO-d₆): 8.73 (d, J=2.4 Hz, 1H), 8.53 (d, J=5.2 Hz, 1H), 7.81 (s, 1H), 7.75 (d, J=5.2 Hz, 1H), 7.44 (s, 1H), 7.39 (s, 1H), 7.21 (d, J=2.4 Hz, 1H), 3.86 (br s, 6H), 3.62 (t, J=4.4 Hz, 4H), 3.53 (t, J=4.4 Hz, 4H), 2.55 (s, 3H), 2.49-2.50 (m, 4H).

### Example 191

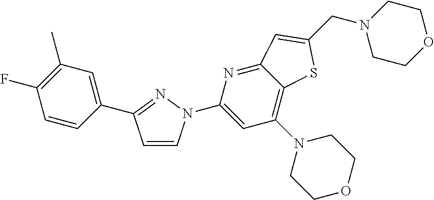 [SMILES: Cc1cc(-c2ccn(-c3cc(N4CCOCC4)c4sc(CN5CCOCC5)cc4n3)n2)ccc1F]

### 4-(5-(3-(4-Fluoro-3-methylphenyl)-1H-pyrazol-1-yl)-2-(morpholinomethyl)-thieno[3,2-b]pyridin-7-yl)morpholine

Following the procedure according to Example 178, 4-(5-(3-bromo-1H-pyrazol-1-yl)-2-(morpholinomethyl)thieno[3,2-b]pyridin-7-yl)morpholine (80 mg) is reacted with (4-fluoro-3-methylphenyl)boronic acid to afford the title compound (40 mg, 47%) as an off white solid. Mass [m/z]=492.3 [M+H]⁺; ¹H-NMR (400 MHz, DMSO-d₆): 8.69 (d, J=2.8 Hz, 1H), 7.92 (d, J=7.6 Hz, 1H), 7.83-7.86 (m, 1H), 7.66 (bs, 1H), 7.51 (s, 1H), 7.22-7.27 (m, 1H), 7.07 (d, J=2.4 Hz, 1H), 4.65 (bs, 2H), 3.87 (t, J=4.0 Hz, 4H), 3.57 (br s, 4H), 3.56 (br s, 4H), 3.17 (br s, 4H), 2.32 (s, 3H).

### Example 192

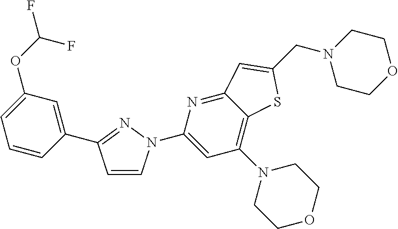 [SMILES: FC(F)Oc1cccc(-c2ccn(-c3cc(N4CCOCC4)c4sc(CN5CCOCC5)cc4n3)n2)c1]

### 4-(5-(3-(3-(Difluoromethoxy)phenyl)-1H-pyrazol-1-yl)-2-(morpholinomethyl)thieno[3,2-b]pyridin-7-ylmorpholine

Following the procedure according to Example 178, 4-(5-(3-bromo-1H-pyrazol-1-yl)-2-(morpholinomethyl)thieno[3,2-b]pyridin-7-yl)morpholine (80 mg) is reacted with (3-(difluoromethoxy)phenyl)boronic acid to afford the title compound (35 mg, 38%) as an off white solid. Mass [m/z]=528.3 [M+H]⁺; ¹H-NMR (400 MHz, DMSO-d₆): 8.72 (d, J=2.4 Hz, 1H), 7.89 (d, J=8.0 Hz, 1H), 7.77 (s, 1H), 7.61 (br s, 1H), 7.16-7.56 (m, 5H), 4.66 (br s, 2H), 3.85-3.87 (m, 6H), 3.70 (br s, 2H), 3.54 (br s, 4H), 3.15 (br s, 4H).

### Example 193

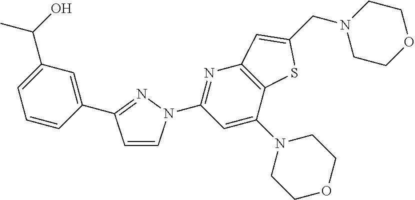 [SMILES: CC(O)c1cccc(-c2ccn(-c3cc(N4CCOCC4)c4sc(CN5CCOCC5)cc4n3)n2)c1]

### 1-(3-(1-(7-Morpholino-2-(morpholinomethyl)thieno[3,2-b]pyridin-5-yl)-1H-pyrazol-3-yl)phenyl)ethan-1-ol

Following the procedure according to Example 178, 4-(5-(3-bromo-1H-pyrazol-1-yl)-2-(morpholinomethyl)thieno[3,2-b]pyridin-7-yl)morpholine (80 mg) is reacted with (3-(1-hydroxyethyl)phenyl)boronic acid to afford the title compound (17 mg, 19%) as an off white solid. Mass [m/z]=506.5 [M+H]⁺; ¹H-NMR (400 MHz, DMSO-d₆): 8.68 (d, J=2.8 Hz, 1H), 7.92 (s, 1H), 7.84 (d, J=7.6 Hz, 1H), 7.35-7.44 (m, 4H),), 7.05 (d, J=2.4 Hz, 1H), 5.25 (d, J=4.0 Hz, 1H), 4.82 (t, J=6.0 Hz, 1H), 3.85 (m, 6H), 3.61 (br d, 4H), 3.51 (br d, 4H), 2.50 (br s, 4H), 1.39 ((d, J=6.4 Hz, 3H).

### Example 194

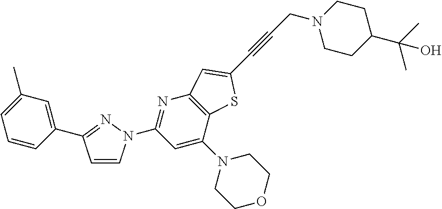 [SMILES: Cc1cccc(-c2ccn(-c3cc(N4CCOCC4)c4sc(C#CCN5CCC(C(C)(C)O)CC5)cc4n3)n2)c1]

### Step 3: 2-(1-(3-(2-Chloro-4-morpholinothieno[3,2-d]pyrimidin-6-yl)prop-2-yn-1-yl)piperidin-4-yl)propan-2-ol

Following the procedure according to Example 181, 4-(2-chloro-6-(3-chloroprop-1-yn-1-yl)thieno[3,2-d]pyrimidin-4-yl)morpholine (75 mg) is reacted with 2-(piperidin-4-yl)propan-2-ol (49 mg) to afford the title compound (70 mg; 70%) as an off-white solid. Mass [m/z]=435.2 [M+H]⁺; ¹H-NMR (400 MHz, DMSO-d₆: 7.56 (s, 1H), 4.05 (s, 1H), 3.8 (t, J=4.4 Hz, 4H), 3.75 (t, J=5.2 Hz, 4H), 3.60 (s, 2H), 2.90 (d, J=10.8 Hz, 2H), 2.13-1.98 (m, 2H), 1.70-1.67 (m, 2H), 1.27-1.04 (m, 3H), 1.02 (s, 6H).

### Step 4: 2-(1-(3-(4-Morpholino-2-(3-(m-tolyl)-1H-pyrazol-1-yl)thieno[3,2-d]pyrimidin-6-yl)prop-2-yn-1-yl)piperidin-4-yl)propan-2-ol

Following the procedure according to Example 181, 2-(1-(3-(2-chloro-4-morpholinothieno[3,2-d]pyrimidin-6-yl)prop-2-yn-1-yl)piperidin-4-yl)propan-2-ol (70 mg) is reacted with 38 mg of 3-(m-tolyl)-1H-pyrazole (38 mg) to afford the title compound (35 mg; 39%) as an off-white solid. Mass [m/z]=557.4 [M+H]⁺; ¹H-NMR (400 MHz, DMSO-d₆): 8.74 (d, J=2.4 Hz, 1H), 7.79 (br s, 1H), 7.75 (d, J=7.6 Hz, 1H), 7.68 (s, 1H), 7.37 (t, J=7.6, 1H), 7.21 (d, J=7.2, 1H), 7.04 (d, J=2.4 Hz, 1H), 4.06 (s, 1H), 4.01 (t, J=4.4 Hz, 4H), 3.81 (t, J=4.4 Hz, 8H), 3.62 (s, 2H), 2.92 (d, J=10.8 Hz, 2H), 2.39 (s, 3H), 2.15-2.10 (m, 2H), 1.71 (d, J=12 Hz, 2H), 1.31-1.10 (m, 3H), 1.03 (s, 6H).

### Example 195

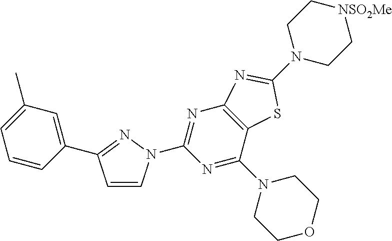

### Step 9: 4-(5-Chloro-2-(4-(methylsulfonyl)piperazin-1-yl)thiazolo[4,5-d]pyrimidin-7-yl)morpholine

Following the procedure according to Example 182, 4-(5-chloro-2-(methylsulfonyl)thiazolo[4,5-d]pyrimidin-7-yl)morpholine (80 mg) and 1-(methylsulfonyl)piperazine (54 mg) are reacted to afford the title compound (65 mg; 65%) as an off-white solid. Mass [m/z]=419.1 [M+H]⁺; ¹H-NMR (400 MHz, DMSO-d₆: 3.77 (br s, 4H), 3.71 (s, 8H), 3.30 (t, J=5.2 Hz, 4H), 2.93 (s, 3H).

### Step 10: 4-(2-(4-(Methylsulfonyl)piperazin-1-yl)-5-(3-(m-tolyl)-1H-pyrazol-1-yl)thiazolo[4,5-d]pyrimidin-7-yl)morpholine

Following the procedure according to Example 182, 4-(5-chloro-2-(4-(methylsulfonyl)piperazin-1-yl)thiazolo[4,5-d]pyrimidin-7-yl)morpholine (30 mg) and 3-(m-tolyl)-1H-pyrazole (11 mg) ae reacted to afford the title compound (2 mg; 5%) as an off-white solid. Mass [m/z]=541.3 [M+H]⁺; ¹H-NMR (400 MHz, DMSO-d₆): 8.68 (d, J=2.8 Hz, 1H), 7.79 (br s, 2H), 7.53 (d, J=8 Hz, 1H), 7.37 (t, J=7.6, 1H), 7.20 (d, J=7.2, 1H), 7.02 (d, J=2.4 Hz, 1H), 3.85 (t, J=4.4 Hz, 4H), 3.81 (br s, 4H), 3.77 (t, J=4.4 Hz, 4H), 3.36 (t, J=4.4 Hz, 4H), 2.94 (s, 3H), 2.39 (s, 3H).

### Example 196

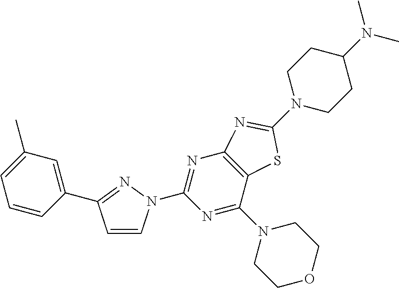 [SMILES: Cc1cccc(-c2ccn(-c3nc(N4CCOCC4)c4sc(N5CCC(N(C)C)CC5)nc4n3)n2)c1]

### Step 9: 1-(5-Chloro-7-morpholinothiazolo[4,5-d]pyrimidin-2-yl)-N,N-dimethyl piperidin-4-amine

Following the procedure according to Example 182, 4-(5-chloro-2-(methylsulfonyl)thiazolo[4,5-d]pyrimidin-7-yl)morpholine (100 mg) is reacted N,N-dimethylpiperidin-4-amine (54 mg) to afford the title compound (80 mg; 70%) as an off-white solid. Mass [m/z]=383.2 [M+H]⁺; ¹H-NMR (400 MHz, DMSO-d₆: 4.21 (br s, 2H), 3.22 (t, J=4.4 Hz, 2H), 2.48-2.43 (m, 1H), 2.30 (s, 6H), 2.2 (s, 6H), 1.96-1.93 (m, 2H), 1.65-1.59 (m, 2H).

### Step 10

Following the procedure according to Example 182, 1-(5-chloro-7-morpholinothiazolo[4,5-d]pyrimidin-2-yl)-N,N-dimethylpiperidin-4-amine (70 mg) and 3-(m-tolyl)-1H-pyrazole (29 mg) were reacted to afford the title compound (12 mg; 13%) as an off white solid. Mass [m/z]=505.4 [M+H]⁺; ¹H-NMR (400 MHz, DMSO-d₆): 8.67 (d, J=2.8 Hz, 1H), 7.79 (br s, 2H), 7.74 (d, J=8 Hz, 1H), 7.36 (t, J=7.6, 1H), 7.20 (d, J=7.2, 1H), 7.01 (d, J=2.4 Hz, 1H), 4.09 (br s, 2H), 3.84 (t, J=4.4 Hz, 4H), 3.76 (t, J=4.4 Hz, 4H), 3.29 (t, J=4.4 Hz, 2H), 2.49-2.42 (m, 1H), 2.39 (s, 3H), 2.2 (s, 6H), 1.90-1.88 (m, 2H), 1.53-1.43 (m, 2H).

### Example 197

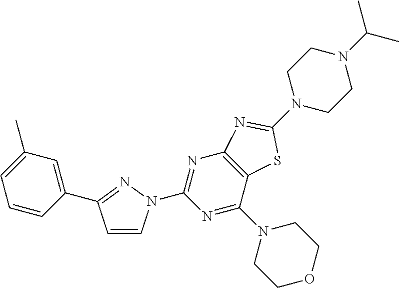 [SMILES: Cc1cccc(-c2ccn(-c3nc(N4CCOCC4)c4sc(N5CCN(C(C)C)CC5)nc4n3)n2)c1]

### Step 9: 4-(5-Chloro-2-(4-isopropylpiperazin-1-yl)thiazolo[4,5-d]pyrimidin-7-yl)morpholine

Following the procedure according to Example 182, 4-(5-chloro-2-(methylsulfonyl)thiazolo[4,5-d]pyrimidin-7-yl)morpholine (100 mg) and 1-isopropylpiperazine (53 mg) are to afford the title compound (85 mg; 74%) as an off-white solid. Mass [m/z]=383.4 [M+H]⁺; ¹H-NMR (400 MHz, DMSO-d₆): 3.70 (s, 8H), 3.61 (br s, 4H), 2.57 (d, J=4.8 Hz, 4H), 0.99 (d, J=6.4 Hz, 6H).

### Step 10: 4-(2-(4-Isopropylpiperazin-1-yl)-5-(3-(m-tolyl)-1H-pyrazol-1-yl)thiazolo[4,5-d]pyrimidin-7-yl)morpholine

Following the procedure according to Example 182, 4-(5-chloro-2-(4-isopropylpiperazin-1-yl)thiazolo[4,5-d]pyrimidin-7-yl)morpholine (60 mg) and 3-(m-tolyl)-1H-pyrazole (25 mg) were reacted to afford the title compound (25 mg; 32%) as an off white solid. Mass [m/z]=505.1 [M+H]⁺; ¹H-NMR (400 MHz, DMSO-d₆): 8.67 (d, J=2.8 Hz, 1H), 7.79 (br s, 2H), 7.74 (d, J=8 Hz, 1H), 7.36 (t, J=7.6, 1H), 7.20 (d, J=7.2 Hz, 1H), 7.01 (d, J=2.4 Hz, 1H), 3.84 (t, J=4.4 Hz, 4H), 3.76 (t, J=4.4 Hz, 8H), 3.65 (br s, 4H), 2.78-2.72 (m, 1H), 2.60 (t, J=4.4 Hz, 4H), 2.39 (s, 3H). 1.01 (d, J=6.8 Hz, 6H).

### Example 198

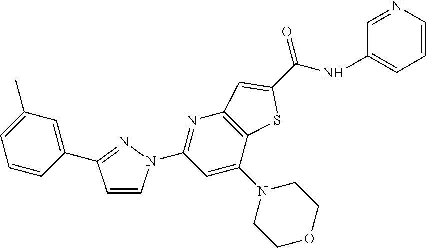 [SMILES: Cc1cccc(-c2ccn(-c3cc(N4CCOCC4)c4sc(C(=O)Nc5cccnc5)cc4n3)n2)c1]

### Step 2: 5-Chloro-7-morpholino-N-(pyridin-3-yl)thieno[3,2-b]pyridine-2-carboxamide

Following the procedure according to Example 177, 5-chloro-7-morpholinothieno[3,2-b]pyridine-2-carboxylic acid (100 mg) is treated with pyridin-3-amine (47 mg) to afford the title compound (80 mg; 64%) as an off-white solid. Mass [m/z]=375 [M+H]⁺; ¹H-NMR (400 MHz, DMSO-d₆): 10.81 (s, 1H), 8.93 (d, J=2.4 Hz, 1H), 8.37 (t, J=1.2 Hz, 2H), 8.18 (d, J=9.2 Hz, 1H), 7.46-7.43 (m, 1H), 6.92 (s, 1H), 3.81 (t, J=4.4 Hz, 4H), 3.53 (t, J=4.8 Hz, 4H).

### Step 3: 7-Morpholino-N-(pyridin-3-yl)-5-(3-(m-tolyl)-1H-pyrazol-1-yl)thieno[3,2-b]pyridine-2-carboxamide

Following the procedure according to Example 177, 5-chloro-7-morpholino-N-(pyridin-3-yl)thieno[3,2-b]pyridine-2-carboxamide (80 mg) and 3-(m-tolyl)-1H-pyrazole (34 mg) are reacted to give to afford the title compound (32 mg; 30%) as an off white solid. Mass [m/z]=497.2 [M+H]⁺; ¹H-NMR (400 MHz, DMSO-d₆): 10.79 (s, 1H), 8.95 (d, J=4 Hz, 1H), 8.72 (d, J=2.4 Hz, 1H), 8.48 (s, 1H), 8.37 (t, J=4 Hz, 1H), 8.21-8.18 (m, 1H), 7.81 (d, J=9.2 Hz, 2H), 7.56 (s, 1H), 7.46-7.43 (m, 1H), 7.39 (t, J=7.6 Hz, 1H), 7.22 (d, J=7.2, 1H), 7.10 (d, J=2.4 Hz, 1H), 3.89 (t, J=4.4 Hz, 4H), 3.59 (t, J=4.8 Hz, 4H), 2.40 (s, 3H).

### Example 199

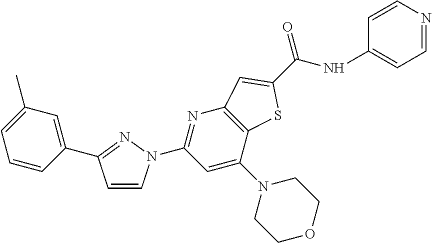 [SMILES: Cc1cccc(-c2ccn(-c3cc(N4CCOCC4)c4sc(C(=O)Nc5ccncc5)cc4n3)n2)c1]

### Step 2: 5-Chloro-7-morpholino-N-(pyridin-4-yl)thieno[3,2-b]pyridine-2-carboxamide

Following the procedure according to Example 177, 5-chloro-7-morpholinothieno[3,2-b]pyridine-2-carboxylic acid (80 mg) is treated with pyridin-4-amine (37 mg) to afford the title compound (60 mg; 60%) as an off-white solid. Mass [m/z]=375 [M+H]⁺; ¹H-NMR (400 MHz, DMSO-d₆): 10.90 (s, 1H), 8.53 (m, 2H), 8.40 (s, 1H), 7.77-7.75 (m, 2H), 6.93 (s, 1H), 3.81 (t, J=4.4 Hz, 4H), 3.53 (t, J=4.8 Hz, 4H).

### Step 3: 7-Morpholino-N-(pyridin-4-yl)-5-(3-(m-tolyl)-1H-pyrazol-1-yl)thieno[3,2-b]pyridine-2-carboxamide

Following the procedure according to Example 177, 5-chloro-7-morpholino-N-(pyridin-4-yl)thieno[3,2-b]pyridine-2-carboxamide (60 mg) and 3-(m-tolyl)-1H-pyrazole (25 mg) were reacted to afford the title compound (10 mg; 12%) as an off white solid. Mass [m/z]=497.2 [M+H]⁺; ¹H-NMR (400 MHz, DMSO-d₆): 10.88 (s, 1H), 8.72 (d, J=2.4 Hz, 1H), 8.54-8.52 (m, 2H), 7.81 (m, 4H), 7.56 (s, 1H), 7.39 (t, J=7.6 Hz, 1H), 7.22 (d, J=7.2 Hz, 1H), 7.10 (d, J=2.4 Hz, 1H), 3.89 (t, J=4.4 Hz, 4H), 3.59 (t, J=4.8 Hz, 4H), 2.40 (s, 3H).

### Example 200

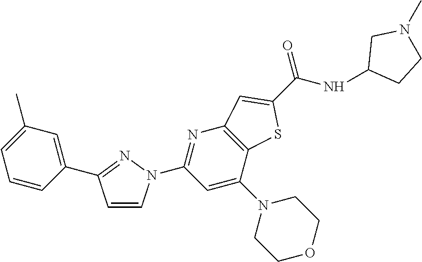 [SMILES: Cc1cccc(-c2ccn(-c3cc(N4CCOCC4)c4sc(C(=O)NC5CCN(C)C5)cc4n3)n2)c1]

### Step 2: 5-Chloro-N-(1-methylpyrrolidin-3-yl)-7-morpholinothieno[3,2-b]pyridine-2-carboxamide

Following the procedure according to Example 177, 5-chloro-7-morpholinothieno[3,2-b]pyridine-2-carboxylic acid (60 mg) is treated with 1-methylpyrrolidin-3-amine (30 mg) to afford the title compound (60 mg; 79%) as an off-white solid. Mass [m/z]=381.1 [M+H]⁺; ¹H-NMR (400 MHz, DMSO-d₆): 9.33 (d, J=4 Hz, 1H), 8.23 (s, 1H), 6.89 (s, 1H), 4.60 (br s, 1H), 3.80 (t, J=4.4 Hz, 4H), 3.50 (t, J=4.8 Hz, 4H), 3.10-3.05 (m, 2H), 2.82 (s, 3H), 1.51 (br s, 1H), 1.23-1.14 (m, 2H).

### Step 3: N-(1-Methylpyrrolidin-3-yl)-7-morpholino-5-(3-(m-tolyl)-1H-pyrazol-1-yl)thieno[3,2-b]pyridine-2-carboxamide

Following the procedure according to Example 177, 5-chloro-N-(1-methylpyrrolidin-3-yl)-7-morpholinothieno[3,2-b]pyridine-2-carboxamide (60 mg) and 25 mg of 3-(m-tolyl)-1H-pyrazole were reacted to afford the title compound (35 mg; 44%) as an off white solid. Mass [m/z]=503.3 [M+H]⁺; ¹H-NMR (400 MHz, DMSO-d₆): 8.89 (d, J=4 Hz, 1H), 8.71 (d, J=2.4 Hz, 1H), 8.2 (s, 1H), 7.83 (d, J=9.2 Hz, 2H), 7.54 (s, 1H), 7.41 (t, J=7.6 Hz, 1H), 7.24 (d, J=7.2, 1H), 7.10 (d, J=2.4 Hz, 1H), 4.44-4.42 (m, 1H), 3.89 (t, J=4.4 Hz, 4H), 3.58 (t, J=4.8 Hz, 4H), 2.77-2.68 (m, 2H), 2.57-2.52 (m, 1H), 2.46-2.44 (m, 1H), 2.42 (s, 3H), 2.34 (s, 3H), 2.26-2.20 (m, 1H), 1.85-1.81 (m, 1H).

### Example 201

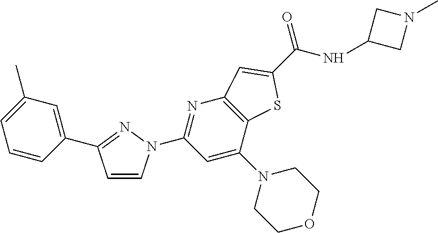 [SMILES: Cc1cccc(-c2ccn(-c3cc(N4CCOCC4)c4sc(C(=O)NC5CN(C)C5)cc4n3)n2)c1]

### Step 2: 5-Chloro-N-(1-methylazetidin-3-yl)-7-morpholinothieno[3,2-b]pyridine-2-carboxamide

Following the procedure according to Example 177, 5-chloro-7-morpholinothieno[3,2-b]pyridine-2-carboxylic acid (60 mg) is treated with 1-methylazetidin-3-amine (26 mg) to afford the title compound (60 mg; 82%) as an off-white solid. Mass [m/z]=367.2 [M+H]; H-NMR (400 MHz, DMSO-d₆): 9.55 (d, J=6.4 Hz, 1H), 8.16 (s, 1H), 6.87 (s, 1H), 4.58-3.95 (m, 1H), 4.03 (t, J=4.4 Hz, 5H), 3.29-3.01 (br s, 2H), 2.39 (s, 3H).

### Step 3: N-(1-Methylazetidin-3-yl)-7-morpholino-5-(3-(m-tolyl)-1H-pyrazol-1-yl)thieno[3,2-b]pyridine-2-carboxamide

Following the procedure according to Example 177, 5-chloro-N-(1-methylazetidin-3-yl)-7-morpholinothieno[3,2-b]pyridine-2-carboxamide (60 mg) and 26 mg of 3-(m-tolyl)-1H-pyrazole (26 mg) to afford the title compound (23 mg; 28%) as an off white solid. Mass [m/z]=489.3 [M+H]⁺; ¹H-NMR (400 MHz, DMSO-d₆): 9.26 (d, J=4 Hz, 1H), 8.72 (d, J=2.4 Hz, 1H), 8.21 (s, 1H), 7.83 (d, J=9.2 Hz, 2H), 7.55 (s, 1H), 7.41 (t, J=7.6 Hz, 1H), 7.24 (d, J=7.2, 1H), 7.11 (d, J=2.4 Hz, 1H), 4.51-4.46 (m, 1H), 3.89 (t, J=4.4 Hz, 4H), 3.66 (t, J=7.2 Hz, 2H), 3.58 (t, J=4.8 Hz, 4H), 3.12 (t, J=7.2 Hz, 4H), 2.42 (s, 3H), 2.33 (s, 3H).

### Example 202

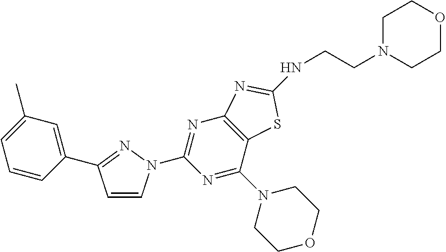 [SMILES: Cc1cccc(-c2ccn(-c3nc(N4CCOCC4)c4sc(NCCN5CCOCC5)nc4n3)n2)c1]

### Step 9: 5-Chloro-7-morpholino-N-(2-morpholinoethyl)thiazolo[4,5-d]pyrimidin-2-amine

Following the procedure according to Example 182, 4-(5-chloro-2-(methylsulfonyl)thiazolo[4,5-d]pyrimidin-7-yl)morpholine (80 mg) is reacted with 2-morpholinoethan-1-amine (43 mg) to afford the title compound (70 mg; 76%) as an off-white solid. Mass [m/z]=385.1 [M+H]⁺; ¹H-NMR (400 MHz, DMSO-d₆: 8.84 (br s, 1H), 3.68-3.67 (m, 8H), 3.58 (t, J=4.4 Hz, 4H), 3.50 (br s, 2H), 2.54-2.51 (m, 2H), 2.42-2.41 (m, 4H).

### Step 10: 7-Morpholino-N-(2-morpholinoethyl)-5-(3-(m-tolyl)-1H-pyrazol-1-yl)thiazolo-[4,5-d]pyrimidin-2-amine

Following the procedure according to Example 182, 5-chloro-7-morpholino-N-(2-morpholinoethyl)thiazolo[4,5-d]pyrimidin-2-amine (70 mg) and 3-(m-tolyl)-1H-pyrazole (29 mg) are reacted to afford the title compound (40 mg; 43%) as an off white solid. Mass [m/z]=507.3 [M+H]⁺; ¹H-NMR (400 MHz, DMSO-d₆): 8.79 (br s, 1H), 8.66 (d, J=2.8 Hz, 1H), 7.79 (br s, 2H), 7.74 (d, J=8 Hz, 1H), 7.36 (t, J=7.6 Hz, 1H), 7.20 (d, J=7.2 Hz, 1H), 7.00 (d, J=2.4 Hz, 1H), 3.80 (t, J=4.4 Hz, 4H), 3.76 (t, J=4.4 Hz, 4H), 3.60-3.57 (m, 6H), 2.41 (s, 3H), 2.58 (t, J=6.4 Hz, 2H), 2.44 (Brs, 4H), 2.39 (s, 3H).

### Example 203

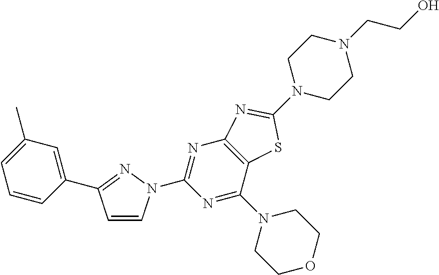 [SMILES: Cc1cccc(-c2ccn(-c3nc(N4CCOCC4)c4sc(N5CCN(CCO)CC5)nc4n3)n2)c1]

### Step 9: 2-(4-(5-Chloro-7-morpholinothiazolo[4,5-d]pyrimidin-2-yl)piperazin-1-yl)ethan-1-ol

Following the procedure according to Example 182, 4-(5-chloro-2-(methylsulfonyl)thiazolo[4,5-d]pyrimidin-7-yl)morpholine (80 mg) is reacted 2-(piperazin-1-yl)ethan-1-ol (44 mg) to afford the title compound (65 mg; 70%) as an off-white solid. Mass [m/z]=385.1 [M+H]⁺; ¹H-NMR (400 MHz, DMSO-d₆: 4.49 (t, J=5.2, 1H), 3.70 (s, 8H), 3.36 (br s, 4H), 3.55-3.51 (m, 2H), 2.67 (t, J=4.4 Hz, 4H), 2.49-2.32 (m, 2H).

### Step 10: 2-(4-(7-Morpholino-5-(3-(m-tolyl)-1H-pyrazol-1-yl)thiazolo[4,5-d]pyrimidin-2-yl)piperazin-1-yl)ethan-1-ol

Following the procedure according to Example 182, 2-(4-(5-chloro-7-morpholinothiazolo[4,5-d]pyrimidin-2-yl)piperazin-1-yl)ethan-1-ol (60 mg) and 3-(m-tolyl)-1H-pyrazole (25 mg) are reacted to afford the title compound (13 mg; 16%) as an off white solid. Mass [m/z]=507.4 [M+H]⁺; ¹H-NMR (400 MHz, DMSO-d₆): 8.67 (d, J=2.8 Hz, 1H), 7.79 (br s, 2H), 7.74 (d, J=8 Hz, 1H), 7.36 (t, J=7.6 Hz, 1H), 7.20 (d, J=7.2 Hz, 1H), 7.01 (d, J=2.4 Hz, 1H), 4.48 (br s, 1H), 3.85 (t, J=4.4 Hz, 4H), 3.76 (t, J=4.4 Hz, 4H), 3.66 (br s, 4H), 3.56 (t, J=5.6 Hz, 2H), 2.67 (t, J=1.6 Hz, 4H), 2.49-2.45 (m, 2H), 2.39 (s, 3H).

### Example 204

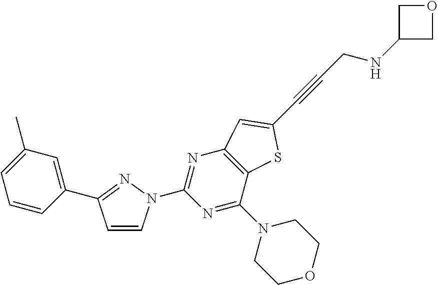 [SMILES: Cc1cccc(-c2ccn(-c3nc(N4CCOCC4)c4sc(C#CCNC5COC5)cc4n3)n2)c1]

### Step 3: N-(3-(2-Chloro-4-morpholinothieno[3,2-d]pyrimidin-6-yl)prop-2-yn-1-yl)oxetan-3-amine

Following the procedure according to Example 181, 4-(2-chloro-6-(3-chloroprop-1-yn-1-yl)thieno[3,2-d]pyrimidin-4-yl)morpholine (50 mg) is reacted with oxetan-3-amine (17 mg) to afford the title compound (35 mg; 63%) as an off-white solid. Mass [m/z]=365.3 [M+H]⁺; ¹H-NMR (400 MHz, CDCl₃): 7.37 (s, 1H), 3.99 (t, J=4.4 Hz, 4H), 3.85 (t, J=5.2 Hz, 4H), 3.65 (s, 2H), 3.31 (Brs, 4H) 2.80 (s, 3H), 2.76 (t, J=4.4 Hz, 4H).

### Step 4: N-(3-(4-Morpholino-2-(3-(m-tolyl)-1H-pyrazol-1-yl)thieno[3,2-d]pyrimidin-6-yl)prop-2-yn-1-yl)oxetan-3-amine

Following the procedure according to Example 181, N-(3-(2-chloro-4-morpholinothieno[3,2-d]pyrimidin-6-yl)prop-2-yn-1-yl)oxetan-3-amine (35 mg) is reacted with 3-(m-tolyl)-1H-pyrazole (23 mg) to afford the title compound (4.6 mg; 10%) as an off-white solid. Mass [m/z]=487.3 [M+H]⁺; ¹H-NMR (400 MHz, DMSO-d₆): 8.74 (d, J=2.4 Hz, 1H), 7.79 (br s, 1H), 7.75 (d, J=7.6 Hz, 1H), 7.65 (s, 1H), 7.37 (t, J=7.6 Hz, 1H), 7.21 (d, J=7.2 Hz, 1H), 7.04 (d, J=2.4 Hz, 1H), 4.70 (t, J=6.8 Hz, 2H), 4.45 (t, J=6.4 Hz, 2H), 3.99 (m, 4H), 3.81 (m, 4H), 3.65 (s, 2H), 2.39 (s, 3H), 1.75 (s, 1H).

### Example 205

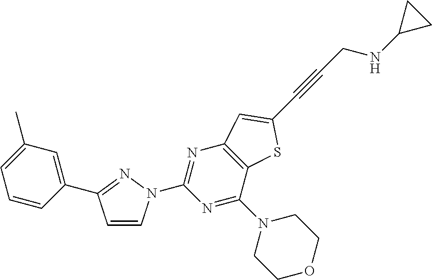 [SMILES: Cc1cccc(-c2ccn(-c3nc(N4CCOCC4)c4sc(C#CCNC5CC5)cc4n3)n2)c1]

### Step 3: N-(3-(2-Chloro-4-morpholinothieno[3,2-d]pyrimidin-6-yl)prop-2-yn-1-yl)cyclopropanamine

Following the procedure according to Example 181, 4-(2-chloro-6-(3-chloroprop-1-yn-1-yl)thieno[3,2-d]pyrimidin-4-yl)morpholine (100 mg) is reacted with cyclopropylamine (16 mg) to afford the title compound (48 mg; 45%) as an off-white solid. Mass [m/z]=349.1 [M+H]⁺; ¹H-NMR (400 MHz, DMSO-d₆: 9.56 (br s, 1H), 7.73 (s, 1H), 4.39 (s, 2H), 3.92 (t, J=4.4 Hz, 4H), 3.79 (t, J=5.2 Hz, 4H), 2.86-2.82 (m, 1H), 0.91-0.80 (m, 4H).

### Step 4

Following the procedure according to Example 181, N-(3-(2-chloro-4-morpholinothieno[3,2-d]pyrimidin-6-yl)prop-2-yn-1-yl)cyclopropanamine (48 mg) is reacted with 3-(m-tolyl)-1H-pyrazole (32 mg) to afford the title compound (6 mg; 19%) as an off-white solid. Mass [m/z]=471.5 [M+H]⁺; ¹H-NMR (400 MHz, DMSO-d₆): 8.74 (d, J=2.4 Hz, 1H), 7.79 (br s, 1H), 7.75 (d, J=7.6 Hz, 1H), 7.65 (s, 1H), 7.37 (t, J=7.6 Hz, 1H), 7.21 (d, J=7.2 Hz, 1H), 7.04 (d, J=2.4 Hz, 1H), 4.00 (t, J=4.4 Hz, 4H), 3.81 (t, J=4.4 Hz, 4H), 3.66 (s, 2H), 2.50 (d, J=1.6 Hz, 1H), 2.39 (s, 3H), 2.33-2.26 (m, 1H), 0.45-0.41 (m, 2H), 0.31-0.28 (m, 2H).

### Example 206

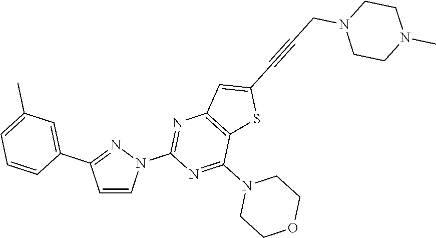 [SMILES: Cc1cccc(-c2ccn(-c3nc(N4CCOCC4)c4sc(C#CCN5CCN(C)CC5)cc4n3)n2)c1]

### Step 3: 4-(2-Chloro-6-(3-(4-methylpiperazin-1-yl)prop-1-yn-1-yl)thieno[3,2-d]pyrimidin-4-yl)morpholine

Following the procedure according to Example 181, 4-(2-chloro-6-(3-chloroprop-1-yn-1-yl)thieno[3,2-d]pyrimidin-4-yl)morpholine (80 mg) is reacted with 1-methylpiperazine (36 mg) to afford the title compound (50 mg; 75%) as an off-white solid. Mass [m/z]=385.1 [M+H]⁺; 1H-NMR (400 MHz, DMSO-d₆: 7.57 (s, 1H), 3.88 (m, 5H), 3.75 (m, 6H), 3.64 (s, 2H), 2.33 (m, 2H), 2.15 (s, 3H).

### Step 4: 4-(6-(3-(4-Methylpiperazin-1-yl)prop-1-yn-1-yl)-2-(3-(m-tolyl)-1H-pyrazol-1-yl)thieno[3,2-d]pyrimidin-4-yl)morpholine

Following the procedure according to Example 181, 4-(2-chloro-6-(3-(4-methylpiperazin-1-yl)prop-1-yn-1-yl)thieno[3,2-d]pyrimidin-4-yl)morpholine (50 mg) is reacted with 3-(m-tolyl)-1H-pyrazole (30 mg) to afford the title compound (15 mg; 23%) as an off-white solid. Mass [m/z]=514.3 [M+H]⁺; 1H-NMR (400 MHz, DMSO-d₆): 8.74 (d, J=2.4 Hz, 1H), 7.79 (br s, 1H), 7.75 (d, J=7.6 Hz, 1H), 7.69 (s, 1H), 7.37 (t, J=7.6 Hz, 1H), 7.21 (d, J=7.2 Hz, 1H), 7.04 (d, J=2.4 Hz, 1H), 4.01 (t, J=4.4 Hz, 4H), 3.81 (t, J=5.2 Hz, 4H), 3.66 (s, 2H), 2.54 (m, 4H), 2.39 (s, 5H), 2.16 (s, 3H), 1.23-1.10 (m, 1H).

### Example 207

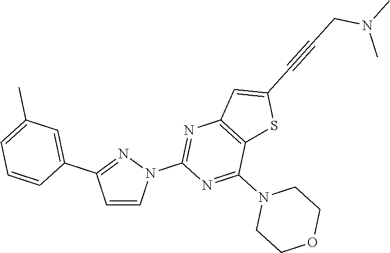 [SMILES: Cc1cccc(-c2ccn(-c3nc(N4CCOCC4)c4sc(C#CCN(C)C)cc4n3)n2)c1]

### Step 3: 3-(2-Chloro-4-morpholinothieno[3,2-d]pyrimidin-6-yl)-N,N-dimethylprop-2-yn-1-amine

Following the procedure according to Example 181, 4-(2-chloro-6-(3-chloroprop-1-yn-1-yl)thieno[3,2-d]pyrimidin-4-yl)morpholine (70 mg) is reacted with dimethylamine (22 mg) to afford the title compound (42 mg; 58%) as an off-white solid. Mass [m/z]=337.1 [M+H]⁺; ¹H-NMR (400 MHz, DMSO-d₆: 7.58 (s, 1H), 3.88 (t, 4H), 3.75 (t, 4H), 3.59 (s, 2H), 2.25 (m, 6H).

### Step 4: N,N-Dimethyl-3-(4-morpholino-2-(3-(m-tolyl)-1H-pyrazol-1-yl)thieno[3,2-d]pyrimidin-6-yl)prop-2-yn-1-amine

Following the procedure according to Example 181, 3-(2-chloro-4-morpholinothieno[3,2-d]pyrimidin-6-yl)-N,N-dimethylprop-2-yn-1-amine (42 mg) is reacted with 3-(m-tolyl)-1H-pyrazole (30 mg) to afford the title compound (22 mg; 38%) as an off white solid. Mass [m/z]=459.2 [M+H]⁺; ¹H-NMR (400 MHz, DMSO-d₆): 8.74 (d, J=2.4 Hz, 1H), 7.79 (br s, 1H), 7.75 (d, J=7.6 Hz, 1H), 7.70 (s, 1H), 7.37 (t, J=7.6, 1H), 7.21 (d, J=7.2, 1H), 7.04 (d, J=2.4 Hz, 1H), 4.01 (t, J=4.4, 4H), 3.81 (t, J=5.2 Hz, 4H), 3.60 (s, 2H), 2.54 (m, 4H), 2.39 (s, 3H), 2.26 (s, 6H).

### Example 208

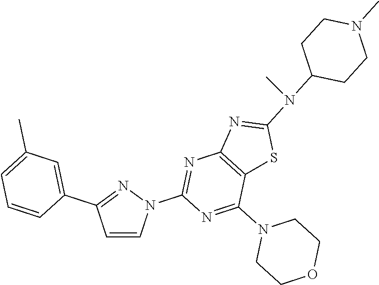 [SMILES: Cc1cccc(-c2ccn(-c3nc(N4CCOCC4)c4sc(N(C)C5CCN(C)CC5)nc4n3)n2)c1]

### Step 9: 5-Chloro-N-methyl-N-(1-methylpiperidin-4-yl)-7-morpholino thiazolo[4,5-d]pyrimidin-2-amine

Following the procedure according to Example 182, 4-(5-chloro-2-(methylsulfonyl)thiazolo[4,5-d]pyrimidin-7-yl)morpholine (80 mg) is reacted with N, 1-dimethylpiperidin-4-amine (43 mg) to afford the title compound (40 mg; 44%) as an off-white solid. Mass [m/z]=383.1 [M+H]⁺; ¹H-NMR (400 MHz, DMSO-d₆: 3.74 (br s, 10H), 3.05 (s, 3H), 2.92-2.90 (m, 2H), 2.23 (s, 3H), 2.085 (br s, 2H), 1.92-1.84 (m, 2H), 1.75-1.67 (m, 2H).

### Step 10: N-Methyl-N-(1-methylpiperidin-4-yl)-7-morpholino-5-(3-(m-tolyl)-1H-pyrazol-1-yl)thiazolo[4,5-d]pyrimidin-2-amine

Following the procedure according to Example 182, 5-chloro-N-methyl-N-(1-methylpiperidin-4-yl)-7-morpholino thiazolo[4,5-d]pyrimidin-2-amine (40 mg) and 3-(m-tolyl)-1H-pyrazole (17 mg) are reacted to afford the title compound (21 mg; 36%) as an off white solid. Mass [m/z]=505.3 [M+H]⁺; ¹H-NMR (400 MHz, DMSO-d₆): 8.70 (d, J=2.8 Hz, 1H), 7.82 (br s, 2H), 7.77 (d, J=8 Hz, 1H), 7.39 (t, J=7.6, 1H), 7.22 (d, J=7.2, 1H), 7.03 (d, J=2.4 Hz, 1H), 3.87 (t, J=4.4 Hz, 4H), 3.79 (t, J=4.4 Hz, 4H), 3.18 (s, 3H), 2.96-2.94 (m, 2H), 2.41 (s, 3H), 2.26 (s, 3H), 2.15-2.10 (m, 2H), 1.97-1.89 (m, 2H), 1.76-1.73 (2H).

### Example 209

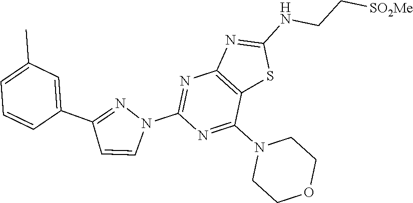 [SMILES: Cc1cccc(-c2ccn(-c3nc(N4CCOCC4)c4sc(NCCS(C)(=O)=O)nc4n3)n2)c1]

### Step 9: 5-Chloro-N-(2-(methylsulfonyl)ethyl)-7-morpholino thiazolo[4,5-d]pyrimidin-2-amine

Following the procedure according to Example 182, 4-(5-chloro-2-(methylsulfonyl)thiazolo[4,5-d]pyrimidin-7-yl)morpholine (80 mg) is reacted with 2-(methylsulfonyl)ethan-1-amine (52 mg) to afford the title compound (50 mg; 55%) as an off-white solid. Mass [m/z]=378.1 [M+H]⁺; ¹H-NMR (400 MHz, DMSO-d₆): 9.14 (br s, 1H), 3.83 (t, J=6.4 Hz, 2H), 3.68 (br s, 8H), 3.50 (t, J=6.4 Hz, 2H), 3.04 (s, 3H).

### Step 10: N-(2-(Methylsulfonyl)ethyl)-7-morpholino-5-(3-(m-tolyl)-1H-pyrazol-1-yl)thiazolo[4,5-d]pyrimidin-2-amine

Following the procedure according to Example 182, 5-chloro-N-(2-(methylsulfonyl)ethyl)-7-morpholino thiazolo[4,5-d]pyrimidin-2-amine (50 mg) and 3-(m-tolyl)-1H-pyrazole (21 mg) are reacted to afford the title compound (3 mg; 5%) as an off white solid. Mass [m/z]=500.42 [M+H]⁺; ¹H-NMR (400 MHz, DMSO-d₆): 9.09 (br s, 1H), 8.67 (d, J=2.4 Hz, 1H), 7.79 (s, 1H), 7.75 (d, J=8 Hz, 1H), 7.36 (t, J=7.6, 1H), 7.19 (d, J=7.2, 1H), 7.06 (d, J=2.4 Hz, 1H), 3.81 (t, J=4.8 Hz, 2H), 3.8 (t, J=4.8 Hz, 4H), 3.76 (t, J=4.8 Hz, 4H), 3.53 (t, J=5.6 Hz, 2H), 3.07 (s, 3H), 2.39 (s, 3H).

### Example 210

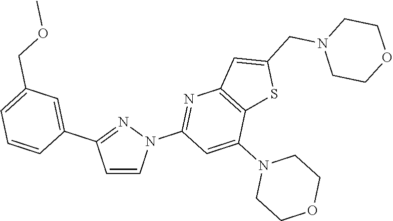 [SMILES: COCc1cccc(-c2ccn(-c3cc(N4CCOCC4)c4sc(CN5CCOCC5)cc4n3)n2)c1]

### 4-(5-(3-(3-(Methoxymethyl)phenyl)-1H-pyrazol-1-yl)-2-(morpholinomethyl)-thieno[3,2-b]pyridin-7-yl)morpholine

To a stirred solution of 4-(5-(3-bromo-1H-pyrazol-1-yl)-2-(morpholinomethyl)thieno[3,2-b]pyridin-7-yl)morpholine (80 mg, 1.0 equiv.) in a mixture of toluene and ethanol (1:1) (10 vol) at rt is added (3-(methoxymethyl)phenyl)boronic acid (2.0 equiv.) followed by K₂CO₃ (2.0 equiv.) and the mixture is degassed with nitrogen for 15 min. PdCl₂(dppf)DCM complex (0.2 eq) is then added and the reaction mixture is degassed with nitrogen for a further 15 min. The reaction mixture is heated to 120° C. and stirred for 16 h. The progress of the reaction is monitored by TLC. After completion, the reaction is diluted with water (10 vol) and extracted with EtOAc (2×10 vol), and the combined organic layers are dried over anhydrous Na₂SO₄, filtered and evaporated under reduced pressure to obtain a crude solid. The crude solid is purified by preparative HPLC eluting with acetonitrile/water to afford the title compound (20 mg, 23%) as an off white solid. Mass [m/z]=506 [M+H]⁺; #¹H-NMR (400 MHz, DMSO-d₆): 8.67 (d, J=2.8 Hz, 1H), 7.90-7.92 (m, 2H), 7.44-7.48 m, 1H), 7.42 (s, 1H), 7.38 (s, 1H), 7.34 (d, J=7.6 Hz, 1H), 7.07 (d, J=2.4 Hz, 1H), 4.50 (s, 2H), 3.83-3.86 (m, 6H), 3.60-3.62 (m, 4H), 3.49-3.52 (m, 4H), 3.34 (s, 3H), 2.46-2.47 (m, 2H).

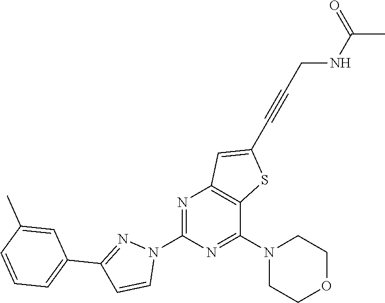 [SMILES: CC(=O)NCC#Cc1cc2nc(-n3ccc(-c4cccc(C)c4)n3)nc(N3CCOCC3)c2s1]

### Example 211

### Step 1: N-(Prop-2-yn-1-yl) acetamide

To a stirred solution of prop-2-yn-1-amine (1 g, 18.18 mmol) in DCM (10 mL), cooled to 0° C., is added triethylamine (3.7 mL, 27.27 mmol, 1.5 eq) and acetyl chloride (2.2 mL, 36.36 mmol, 2 eq). The reaction mixture is stirred at room temperature for 3 h and then it is diluted with EtOAc (30 mL). The organic layer is washed with water (20 mL), dried over anhydrous Na₂SO₄, filtered and evaporated under reduced pressure to provide a crude liquid which is purified by silica gel chromatography to afford the title compound (450 mg; 25%) as a colorless liquid. ¹HNMR 400 MHz, DMSO-d₆: 8.26 (s, 1H), 3.83-3.81 (m, 2H), 3.08 (t, J=2.4 Hz, 1H), 1.81 s, 3H).

### Step 2: N-(3-(2-Chloro-4-morpholinothieno[3,2-d]pyrimidin-6-yl)prop-2-yn-1-yl)acetamide

To a stirred solution of 4-(2-chloro-6-iodothieno[3,2-d]pyrimidin-4-yl)morpholine (200 mg, 0.52 mmol) in THE (10 mL) is added N-(prop-2-yn-1-yl) acetamide (76 mg, 0.78 mmol, 1.5 eq), CuI (5 mg, 0.026 mmol, 0.05 eq), and triethylamine (3.4 mL, 23.67 mmol, 45 eq), followed by Pd(dppf)C₁₂ (18 mg, 0.026 mmol, 0.05 eq) at room temperature. The reaction mixture is stirred at room temperature for 8 h and then diluted with EtOAc (30 mL). The organic layer is washed with water (15 mL), dried over anhydrous Na₂SO₄, filtered and evaporated under reduced pressure to provide a brown gummy residue which is purified by reverse phase flash chromatography to afford the title compound (100 mg; 54%) as a light brown solid. Mass [m/z]=351.3 [M+H]⁺; ¹H-NMR (400 MHz, DMSO-d₆: 8.49 (t, J=5.2 Hz, 1H), 7.59 (s, 1H), 4.22 (d, J=5.6 Hz, 2H), 3.95-3.88 (m, 4H), 3.79-3.75 (m, 4H), 1.88 (s, 3H).

### Step 3: 4-(3-(4-Morpholino-2-(3-(m-tolyl)-1H-pyrazol-1-yl)thieno[3,2-d]pyrimidin-6-yl)prop-2-yn-1-yl)morpholine

To a solution of compound N-(3-(2-chloro-4-morpholinothieno[3,2-d]pyrimidin-6-yl)prop-2-yn-1-yl)acetamide (100 mg, 0.28 mmol, 1 eq) in a mixture of 1,4-dioxane and toluene (1:1, 5 mL), is added 3-(m-tolyl)-1H-pyrazole (67 mg, 0.428 mmol, 1.5 eq) and K₃PO₄ (121 mg, 0.57 mmol, 2 eq). The reaction mixture is degassed with nitrogen for 15 min followed by the addition of Pd₂(dba)₃ (26 mg, 0.028 mmol, 0.1 eq) and ^{t}Bu-XPhos (24 mg, 0.057 mmol, 0.2 eq) at room temperature. The reaction mixture is heated to 150° C. and stirred for 16 h. The reaction is cooled to room temperature, filtered through a celite pad, and the pad is washed with ethyl acetate. The filtrate is evaporated under reduced pressure and the resulting brown gum is purified by preparative HPLC purification eluting with acetonitrile/water to afford the title compound (26 mg; 19%) as an off white solid. Mass [m/z]=473.2 [M+H]⁺; ¹H-NMR (400 MHz, DMSO-d₆): 8.72 (d, J=2.8 Hz, 1H), 7.79 (br s, 2H), 7.55 (d, J=8 Hz, 1H), 7.39 (s, 1H), 7.35 (t, J=7.6 Hz, 1H), 7.20 (d, J=7.2 Hz, 1H), 7.02 (d, J=2.4 Hz, 1H), 6.95 (s, 1H), 4.44 (s, 2H), 3.98 (t, J=4.4 Hz, 4H), 3.79 (t, J=4.4 Hz, 4H), 2.39 (s, 3H), 2.37 (s, 3H).

### Examples 212 and 213

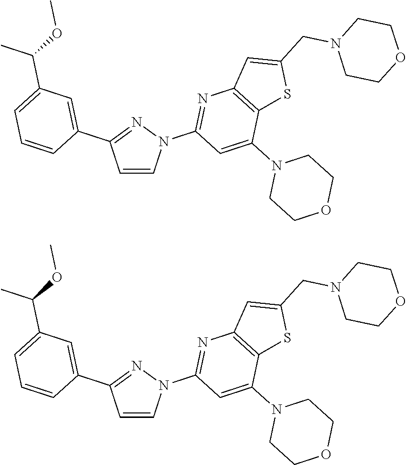 [SMILES: CO[C@@H](C)c1cccc(-c2ccn(-c3cc(N4CCOCC4)c4sc(CN5CCOCC5)cc4n3)n2)c1 ; CO[C@H](C)c1cccc(-c2ccn(-c3cc(N4CCOCC4)c4sc(CN5CCOCC5)cc4n3)n2)c1]

### 4-(5-(3-(3-(1-methoxyethyl)phenyl)-1H-pyrazol-1-yl)-2-(morpholinomethyl)thieno[3,2-b]pyridin-7-yl)morpholine (Two Enantiomers)

Following the procedure according to Example 210, 4-(5-(3-bromo-1H-pyrazol-1-yl)-2-(morpholinomethyl)thieno[3,2-b]pyridin-7-yl)morpholine is reacted with (3-(1-methoxyethyl)phenyl)boronic acid to provide a crude solid which purified by silica gel (using 60-120 mesh) column eluting with 2% MeOH/DCM to afford a racemic product. The racemic product is further purified by chiral separation (ChiralPak-IG (4.6×250 mm), 5 micron; eluting with 0.1% TFA-hexane/ethanol 70:30 v/v at 1.5 mL/min) to afford the title compound as Isomer 1 (Ex. 212) (43 mg) and Isomer 2 (Ex. 213) (43 mg) as solids.

Isomer 1 (Ex. 212): Mass [m/z]=520.5 [M+H]⁺; ¹H-NMR (400 MHz, DMSO-d₆): 8.71 (d, J=2.4 Hz, 1H), 7.88-7.92 (m, 2H), 7.70 (s, 1H), 7.52 (s, 1H), 7.47 (d, J=7.6 Hz, 1H), 7.34 (d, J=7.6 Hz, 1H), 7.11 (d, J=2.4 Hz, 1H), 4.71 (s, 2H), 4.38-4.43 (m, 1H), 3.74 (br s, 4H), 3.54-3.57 (m, 6H), 3.29 (br s, 2H), 3.17 (s, 3H), 1.40 (d, J=6.4 Hz, 3H). Rotation[α]²⁵₅₈₉=−19.88 (C=0.5% w/v EtOH). #

Isomer 2 (Ex. 213): Mass [m/z]=520.5 [M+H]⁺; #¹H-NMR (400 MHz, DMSO-d₆): 8.71 (d, J=2.8 Hz, 1H), 7.88-7.92 (m, 2H), 7.68 (s, 1H), 7.52 (s, 1H), 7.45-7.49 (m, 2H), 7.34 (d, J=7.6 Hz, 1H), 7.11 (d, J=2.4 Hz, 1H), 4.68 (s, 2H), 4.38-4.43 (m, 1H), 3.85-3.87 (br s, 4H), 3.73 (br s, 2H), 3.54-3.56 (m, 4H), 3.34 (br s, 2H), 3.17 (s, 3H), 1.40 (d, J=6.4 Hz, 3H). Rotation[α]²⁵₅₈₉=+16.43 (C=0.5% w/v EtOH). #

### Examples 214 and 215

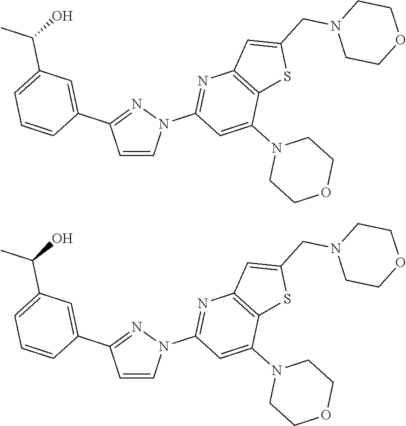

### 1-(3-(1-(7-morpholino-2-(morpholinomethyl)thieno[3,2-b]pyridin-5-yl)-1H-pyrazol-3-yl)phenyl)ethan-1-ol (Two Enantiomers)

Following the procedure according to Example 212/213, 4-(5-(3-bromo-1H-pyrazol-1-yl)-2-(morpholinomethyl)thieno[3,2-b]pyridin-7-yl)morpholine is reacted with (3-(1-hydroxyethyl)phenyl)boronic acid to afford a racemic product, which is purified by chiral separation (ChiralPak-IG (4.6×250 mm), 5 micron; eluting with hexane/ethanol 50:50 v/v at 1.5 mL/min) to afford the title compound as Isomer 1 (Ex. 214) (21 mg) and Isomer 2 (Ex. 215) (25 mg) as solids.

Isomer 1 (Ex. 214): Mass [m/z]=506.2 [M+H]⁺; #¹H-NMR (400 MHz, DMSO-d₆): 8.67 (d, J=2.8 Hz, 1H), 7.92 (s, 1H), 7.83 (d, J=7.6 Hz, 1H), 7.35-7.44 (m, 4H), 7.04 (d, J=2.4 Hz, 1H), 5.23 (d, J=4.0 Hz, 1H), 4.79-4.82 (m, 1H), 3.84-3.86 (m, 6H), 3.61 (br s, 4H), 3.49-3.52 (m, 4H), 2.52 (br s, 4H), 1.38 (d, J=6.8 Hz, 3H). Rotation[α]²⁵₅₈₉=+13.96 (C=0.5% w/v EtOH). #

Isomer 2 (Ex. 215): Mass [m/z]=506.3 [M+H]⁺; ¹H-NMR (400 MHz, DMSO-d₆): 8.71 (d, J=2.4 Hz, 1H), 7.95 (s, 1H), 7.86 (d, J=7.6 Hz, 1H), 7.38-7.49 (m, 4H), 7.09 (d, J=2.4 Hz, 1H), 5.26 (br s, 1H), 4.80-4.85 (m, 1H), 3.87-3.89 (m, 6H), 3.66 (br s, 4H), 3.55 (br s, 4H), 2.53 (br s, 4H), 1.41 (d, J=6.4 Hz, 3H). Rotation[α]²⁵₅₈₉=+11.88 (C=0.5% w/v EtOH). #

### Example 216

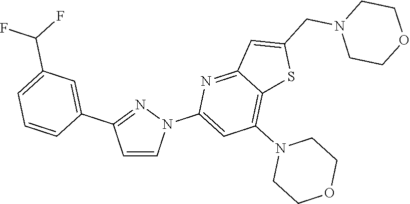 [SMILES: FC(F)c1cccc(-c2ccn(-c3cc(N4CCOCC4)c4sc(CN5CCOCC5)cc4n3)n2)c1]

### 4-(5-(3-(3-(Difluoromethyl)phenyl)-1H-pyrazol-1-yl)-2-(morpholino-methyl)thieno[3,2-b]pyridin-7-yl)morpholine

Following the procedure according to Example 210, 4-(5-(3-bromo-1H-pyrazol-1-yl)-2-(morpholinomethyl)thieno[3,2-b]pyridin-7-yl)morpholine (80 mg) is reacted with (3-(difluoromethyl)phenyl)boronic acid to provide a crude solid which is purified by preparative HPLC eluting with acetonitrile/water to afford the title compound (31 mg, 35%) as an off white solid. Mass [m/z]=512.3 [M+H]⁺; #¹H-NMR (400 MHz, DMSO-d₆): 8.71 (d, J=2.8 Hz, 1H), 8.16 (br s, 2H), 7.58-7.66 (m, 2H), 7.43 (s, 1H), 7.38 (s, 1H), 6.99-7.27 (m, 2H), 3.83-3.86 (m, 6H), 3.60-3.62 (m, 4H), 3.50-3.52 (m, 4H), 2.50 (br s, 4H). #

### Example 217

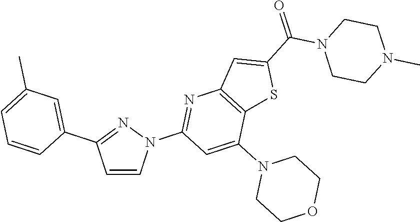 [SMILES: Cc1cccc(-c2ccn(-c3cc(N4CCOCC4)c4sc(C(=O)N5CCN(C)CC5)cc4n3)n2)c1]

### Step 2: (5-chloro-7-morpholinothieno[3,2-b]pyridin-2-yl)(4-methylpiperazin-1-yl)methanone

Following the procedure according to Example 177, 5-chloro-7-morpholinothieno[3,2-b]pyridine-2-carboxylic acid is treated with 1-methylpiperazine to afford the title compound.

### Step 2: (4-Methylpiperazin-1-yl)(7-morpholino-5-(3-(m-tolyl)-1H-pyrazol-1-yl)thieno[3,2-b]pyridin-2-yl)methanone

Following the procedure according to Example 177, (5-chloro-7-morpholinothieno[3,2-b]pyridin-2-yl)(4-methylpiperazin-1-yl)methanone and of 3-(m-tolyl)-1H-pyrazole are reacted to afford the title compound.

### Example 217

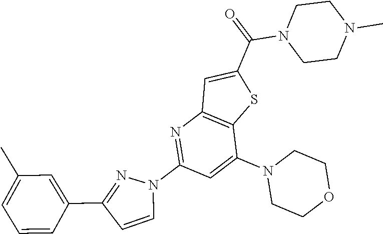

### Step 1: 4-(5-Chlorothieno[3,2-b]pyridin-7-yl)morpholine

To a stirred solution of 5,7-dichlorothieno[3,2-b]pyridine (4.0 g, 19.80 mmol) in ethylene glycol (20 mL) is added morpholine (3.4 mL, 39.6 mmol, 2.0 eq.) followed by triethylamine (5.7 mL, 39.6 mmol, 2.0 eq.). The reaction temperature is raised to 120° C. and stirred for 8 h. The progress of the reaction is monitored by TLC. After completion of the reaction, the reaction is diluted with H₂O (30 mL), filtered, washed with water, and dried to obtain a brown solid which is triturated with diethyl ether to afford the title compound (2.7 g, 66%) as brown solid. Mass [m/z]=254.88 [M+H]⁺; ¹H-NMR (400 MHz, CDCl₃): 7.65 (d, J=5.2 Hz, 1H), 7.45 (d, J=5.2 Hz, 1H), 6.65 (s, 1H), 3.92 (t, J=4.8 Hz, 4H), 3.47 (t, J=4.8 Hz, 4H).

### Step 2: 5-Chloro-7-morpholinothieno[3,2-b]pyridine-2-carboxylic acid

To a stirred solution of 4-(5-chlorothieno[3,2-b]pyridin-7-yl)morpholine (2.0 g, 7.87 mmol) in THE (30 mL) is added n-BuLi (2.5M) (6.3 mL, 15.74 mmol, 2 eq) at −78° C. The reaction mixture is stirred for 1 h at −78° C. then the reaction mass is quenched with dry ice and stirred for 30 min at 0° C. Progress of the reaction is monitored by TLC. After completion of the reaction, it is quenched with saturated NH₄Cl solution (20 mL) and extracted with EtOAc (2×50 mL). The organic layer is washed with water (20 mL), dried over anhydrous Na₂SO₄, filtered, and evaporated under reduced pressure to provide a crude solid which is triturated with diethyl ether to afford the title compound (1.1 g, 48%) as brown solid. Mass [m/z]=299.0 [M+H]⁺; ¹H-NMR (400 MHz, DMSO-d₆): 13.89 (br s, 1H), 7.98 (s, 1H), 6.94 (s, 1H), 3.81 (t, J=4.4 Hz, 4H), 3.52 (t, J=4.8 Hz, 4H).

### Step 3: (5-Chloro-7-morpholinothieno[3,2-b]pyridin-2-yl)(4-methylpiperazin-1-yl) methanone

To a stirred solution of 5-chloro-7-morpholinothieno[3,2-b]pyridine-2-carboxylic acid (200 mg, 0.67 mmol) in DCM (5 mL) is added N-methylpiperazine (80 mg, 0.80 mmol, 1.2 eq) followed by HATU (380 mg, 1.05 mmol, 1.5 eq) and DIPEA (0.25 mL, 1.34 mmol, 2.0 eq.) at room temperature. The reaction mixture is stirred at room temperature for 16 h and the progress of the reaction is monitored by TLC. After completion, the reaction is diluted with DCM, washed with water (5 mL), and brine solution (5 mL), and the organics are dried over Na₂SO₄, filtered, and evaporated under vacuum to provide a crude compound, which is purified on silica gel (60-120 mesh) eluting with 40-60% EtOAc/hexane to afford the title compound (120 mg, 47%) as an off-white solid. Mass [m/z]=381.28 [M+H]⁺; ¹H-NMR (400 MHz, CDCl₃): 7.55 (s, 1H), 6.71 (s, 1H), 3.94 (t, J=4.8 Hz, 4H), 3.83 (br s, 4H), 3.51 (t, J=4.8 Hz, 4H), 2.52 (br s, 4H), 2.39 (s, 3H).

### Step 4: (4-Methylpiperazin-1-yl)(7-morpholino-5-(3-(m-tolyl)-1H-pyrazol-1-yl) thieno[3,2-b]pyridin-2-yl)methanone

To a stirred solution of (5-chloro-7-morpholinothieno[3,2-b]pyridin-2-yl)(4-methylpiperazin-1-yl)methanone (80 mg, 0.21 mmol) in 1,4-dioxane:toluene (2 mL, 1:1) is added 3-(m-tolyl)-1H-pyrazole (40 mg, 0.25 mmol, 1.2 eq), and K₃PO₄ (90 mg, 0.42 mmol, 2 eq) at room temperature. The reaction mixture is degassed for 15 min with nitrogen followed by the addition of Pd₂(dba)₃ (20 mg, 0.021 mmol, 0.1 eq) and t-Butyl-XPhos (18 mg, 0.042 mmol, 0.2 eq), and the reaction is degassed again for 15 min with nitrogen. The reaction temperature is raised to 120° C. and it is stirred for 16 h. The progress of the reaction is monitored by TLC. After completion, the reaction mixture is cooled to room temperature and filtered through a celite pad, the pad is washed with ethyl acetate (5 mL) and the filtrate is concentrated under reduced pressure to provide a crude solid which is purified by Preparative HPLC (Method-: X-BRIDGE C18 (250×21.22 mm) 5 μm; Mobile phase: [ACN: 0.15% of TFA in Water]; time/B %: 0/20, 20/60, 21/60), Flow rate: 14 mL/min) to afford the title compound (18 mg, 17%) as an off-white solid. Mass [m/z]=503.3 [M+H]⁺; ¹H-NMR (400 MHz, DMSO-d₆): 10.03 (br s, 1H), 8.69 (d, J=2.8 Hz, 1H), 7.89 (s, 1H), 7.83 (s, 1H), 7.81 (s, 1H), 7.57 (s, 1H), 7.39 (t, J=7.6 Hz, 1H), 7.24 (d, J=8.0 Hz, 1H), 7.11 (d, J=2.8 Hz, 1H), 4.49 (br s, 2H), 3.88 (t, J=4.0 Hz, 4H), 3.58 (br s, 8H), 3.19-3.20 (m, 2H), 2.88 (s, 3H), 2.42 (s, 3H).

### Example 218

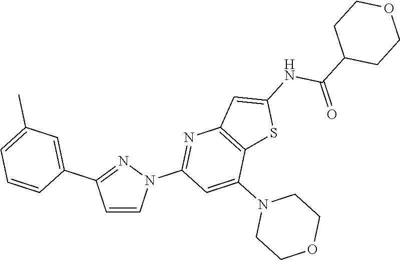 [SMILES: Cc1cccc(-c2ccn(-c3cc(N4CCOCC4)c4sc(NC(=O)C5CCOCC5)cc4n3)n2)c1]

### Step 1: 5-Chloro-7-morpholinothieno[3,2-b]pyridine-2-carboxylic acid

To a stirred solution of 4-(5-chlorothieno[3,2-b]pyridin-7-yl)morpholine (30 mg, 0.118 mmol) in dry THE (5 mL) at −78° C. is added n-BuLi (2.5M in hexane) (0.07 mL, 0.177 mmol, 2 eq) and the reaction is stirred for 1 h. Dry ice is then added and then the reaction is stirred at 0° C. for 1 h after which the reaction is quenched with saturated NH₄C₁ solution (5 mL) and extracted with EtOAc (2×20 mL). The organic layer is dried over anhydrous Na₂SO₄, filtered, and evaporated under reduced pressure to afford the title compound (20 mg: 57%) as an off-white solid. Mass [m/z]=299.0 [M+H]⁺; ¹H-NMR (400 MHz, DMSO-d₆): 7.32 (s, 1H), 6.73 (s, 1H), 3.81 (t, J=4.4 Hz, 4H), 3.48 (t, J=4.8 Hz, 4H).

### Step 2: tert-Butyl (5-chloro-7-morpholinothieno[3,2-b]pyridin-2-yl) carbamate

To a stirred solution of 5-chloro-7-morpholinothieno[3,2-b]pyridine-2-carboxylic acid (900 mg, 3.02 mmol) in tert-butyl alcohol (10 mL) is added diphenylphosphoryl azide (1.66 g, 6.04 mmol, 1 eq.) and triethylamine (0.9 mL, 6.04 mmol, 1 eq.). The reaction mixture is stirred at 80° C. for 12 h. The reaction solvent is evaporated and the obtained residue is diluted with ethyl acetate (50 mL). The organic layer is washed with water (20 mL), and then with brine solution (20 mL), and is dried over Na₂SO₄, filtered, and evaporated under reduced pressure to obtain a sticky gel which is purified by silica gel purification (60-120 mesh) eluting with 50:50 EtOAc/hexane to afford the title compound (480 mg; 43%) as an off-white solid. Mass [m/z]=370.2 [M+H]⁺; ¹H-NMR (400 MHz, DMSO-d₆): 7.40 (br s, 1H), 6.71 (s, 1H), 6.55 (s, 1H), 3.90 (t, J=4.8 Hz, 4H), 3.42 (t, J=4.8 Hz, 4H), 1.55 (s, 9H).

### Step 3: tert-Butyl (7-morpholino-5-(3-(m-tolyl)-1H-pyrazol-1-yl)thieno[3,2-b]pyridin-2-yl)carbamate

To a stirred solution of tert-butyl (5-chloro-7-morpholinothieno[3,2-b]pyridin-2-yl)carbamate (400 mg, 1.08 mmol), and 3-(m-tolyl)-1H-pyrazole (170 mg, 1.08 mmoL, 1.0 eq.) in a mixture of toluene and 1,4-dioxane (1:1, 6 mL) is added Pd₂(dba)₃ (99 mg, 0.108 mmol, 0.1 eq.), and ^{t}Bu-XPhos (92 mg, 0.216 mmol, 0.2 eq.), followed by K₃PO₄ (450 mg, 2.16 mmol, 2 eq.), and the reaction is degassed with nitrogen for 30 min. The reaction mixture is then heated to 140° C. for 24 h. The progress of the reaction is monitored by TLC. After completion of the reaction, the solids are filtered, and the filtrate is diluted with EtOAc (30 mL). The organic layer is washed with water (10 mL), dried over anhydrous Na₂SO₄, filtered and evaporated under reduced pressure to provide a light brown liquid which is purified by reverse phase flash chromatography (C18) eluting with 35-45% acetonitrile/water to afford the title compound (80 mg; 15%) as an off white solid. Mass [m/z]=492.3 [M+H]⁺; ¹H-NMR (400 MHz, DMSO-d₆): 11.03 (br s, 1H), 8.66 (d, J=2.8 Hz, 1H), 7.78-7.75 (m, 2H), 7.37-7.33 (m, 2H), 7.20 (d, J=7.6, 1H), 7.02 (d, J=2.4 Hz, 1H), 6.76 (s, 1H), 3.86 (t, J=4.4 Hz, 4H), 3.46 (t, J=4.8 Hz, 4H), 2.33 (s, 3H), 1.51 (s, 9H).

### Step 4: 7-Morpholino-5-(3-(m-tolyl)-1H-pyrazol-1-yl)thieno[3,2-b]pyridin-2-amine

To a stirred solution of tert-butyl (7-morpholino-5-(3-(m-tolyl)-1H-pyrazol-1-yl)thieno[3,2-b]pyridin-2-yl)carbamate (150 mg, 0.305 mmol) in DCM (5 mL) is added TFA (0.12 mL, 1.52 mmol, 5 eq) at 0° C. The reaction mixture is warmed to room temperature and stirred for 4 h, and then it is quenched with saturated aqueous NaHCO₃ solution (10 mL). The mixture is extracted with EtOAc (2×20 mL), and the organic layer is dried over anhydrous Na₂SO₄, filtered and evaporated under reduced pressure to provide a crude off-white sticky solid. Then crude product is purified by reverse phase flash chromatography (C18 column, 6 g) eluting with 25-35% acetonitrile/water to afford the title compound (80 mg; 67%) as an off-white solid. Mass [m/z]=392.3 [M+H]⁺; ¹H-NMR (400 MHz, DMSO-d₆): 8.60 (d, J=2.8 Hz, 1H), 7.77-7.73 (m, 2H), 7.36 (t, J=7.2 Hz, 1H), 7.23 (d, J=7.6, 1H), 7.15 (s, 1H), 6.98 (d, J=2.4 Hz, 1H), 6.62 (s, 2H), 6.03 (s, 1H), 3.81 (t, J=4.4 Hz, 4H), 3.36 (t, J=4.8 Hz, 4H), 2.39 (s, 3H).

### Step 5: N-(7-morpholino-5-(3-(m-tolyl)-1H-pyrazol-1-yl)thieno[3,2-b]pyridin-2-yl)tetrahydro-2H-pyran-4-carboxamide

To a stirred solution of 7-morpholino-5-(3-(m-tolyl)-1H-pyrazol-1-yl)thieno[3,2-b]pyridin-2-amine (50 mg, 0.127 mmol, 1 eq.) in DCM (2 mL) is added triethylamine (0.04 mL, 0.255 mmol, 2 eq.) at 0° C. followed by a solution of tetrahydro-2H-pyran-4-carbonyl chloride (37 mg, 0.255 mmol, 2 eq.) dissolved in DCM (4 mL). The reaction temperature is warmed to room temperature and stirred for 5 h. The reaction is quenched with water (20 mL) and extracted with EtOAc (30 mL). The organic layer is dried over anhydrous Na₂SO₄, filtered and evaporated under reduced pressure to provide an off-white sticky gel. The crude product is purified by preparative HPLC eluting with acetonitrile/water to afford the title compound (19 mg; 30%) as an off white solid. Mass [m/z]=504.5 [M+H]⁺; ¹H-NMR (400 MHz, DMSO-d₆): 11.68 (s, 1H), 8.70 (d, J=2.8 Hz, 1H), 7.8 (t, J=5.2 Hz, 2H), 7.38-7.34 (m, 2H), 7.21 (d, J=7.2, 1H), 7.05 (d, J=2.4 Hz, 1H), 6.95 (s, 1H), 3.94-3.91 (m, 2H), 3.86 (t, J=4.4 Hz, 4H), 3.53 (t, J=4.8 Hz, 4H), 2.71-2.67 (m, 1H), 2.39 (s, 3H), 1.79-1.66 (m, 4H).

### Example 268

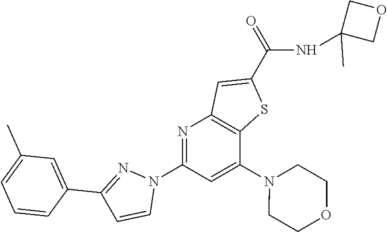 [SMILES: Cc1cccc(-c2ccn(-c3cc(N4CCOCC4)c4sc(C(=O)NC5(C)COC5)cc4n3)n2)c1]

### Step 1: 5-Chloro-N-(3-methyloxetan-3-yl)-7-morpholinothieno[3,2-b]pyridine-2-carboxamide

To a stirred solution of 5-chloro-7-morpholinothieno[3,2-b]pyridine-2-carboxylic acid (80 mg, 1 eq.), is added oxetane-3-amine (35 mg, 1.5 eq.) in DCM (10 vol), followed by T3P (1.5 eq.) and triethylamine (2.0 eq.). The reaction is stirred at room temperature for 18 h and the progress of the reaction is monitored by TLC. After completion, the reaction is diluted with ethyl acetate (20 vol), washed with ice cold water (2×15 vol) and brine solution (20 vol). Finally, the organic layers are dried over Na₂SO₄, filtered, and evaporated under reduced vacuum to provide a crude residue which is purified by silica gel chromatography (60-120 mesh) eluting with 40-80% EtOAc/hexane to afford the title compound (40 mg; 41%) as an off-white solid. Mass [m/z]=368.3 [M+H]⁺; ¹H-NMR (400 MHz, DMSO-d₆): 9.31 (s, 1H), 8.06 (s, 1H), 6.88 (s, 1H), 4.74 (d, J=6.4 Hz, 2H), 4.41 (d, J=6.4 Hz, 2H), 3.80 (t, J=4.4 Hz, 4H), 3.50 (t, J=4.8 Hz, 4H), 1.63 (s, 3H).

### Step 2: N-(3-methyloxetan-3-yl)-7-morpholino-5-(3-(m-tolyl)-1H-pyrazol-1-yl)thieno[3,2-b]pyridine-2-carboxamide

To a solution of 5-chloro-N-(3-methyloxetan-3-yl)-7-morpholinothieno[3,2-b]pyridine-2-carboxamide (40 mg, 1 eq) in a mixture of 1,4-dioxane:toluene (20 vol, 1:1) is added 3-(m-tolyl)-1H-pyrazole (17 mg, 1.0 eq), K₃PO₄ (2 eq). The reaction mixture is degassed for 15 min under nitrogen followed by the addition of Pd₂(dba)₃ (0.1 eq) and t-Bu-XPhos (0.2 eq) at room temperature. The reaction temperature is raised to 150° C. and it is stirred for 24 h. The progress of the reaction is monitored by TLC. After completion of the reaction, it is cooled down to room temperature and filtered through a celite pad, and the pad is washed with ethyl acetate (100 vol). The filtrate is concentrated under reduced pressure to provide a brown gummy solid residue which is purified by reverse phase flash chromatography eluting with 55-60% acetonitrile/water to afford the title compound (17 mg; 32%) as an off white solid. Mass [m/z]=490.2 [M+H]⁺; ¹H-NMR (400 MHz, DMSO-d₆): 9.29 (s, 1H), 8.70 (d, J=2.8 Hz, 1H), 8.18 (s, 1H), 7.80-7.78 (m, 2H), 7.52 (s, 1H), 7.39 (t, J=2.4 Hz, 1H), 7.22 (d, J=7.2 Hz, 1H), 7.09 (d, J=2.4 Hz, 1H), 4.76 (d, J=6.4 Hz, 2H), 4.43 (d, J=6.4 Hz, 2H), 3.87 (t, J=4.4 Hz, 4H), 3.56 (t, J=4.8 Hz, 4H), 2.39 (s, 3H), 1.64 (s, 3H). #

### Example 269

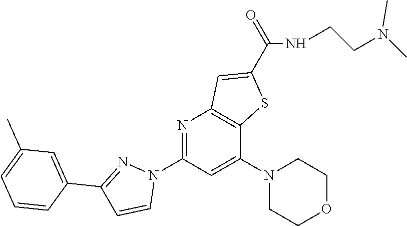 [SMILES: Cc1cccc(-c2ccn(-c3cc(N4CCOCC4)c4sc(C(=O)NCCN(C)C)cc4n3)n2)c1]

### Step 1: 5-Chloro-N-(2-(dimethylamino)ethyl)-7-morpholinothieno[3,2-b]pyridine-2-carboxamide

Following the procedure according to Example 268, 5-chloro-7-morpholinothieno[3,2-b]pyridine-2-carboxylic acid (100 mg) is reacted with N,N-dimethylethane-1,2-diamine (44 mg) to provide a crude residue which is purified by silica gel chromatography (60-120 mesh) eluting with 50% EtOAc/hexane to afford the title compound (55 mg; 45%) as an off-white solid. Mass [m/z]=369.4 [M+H]⁺; ¹H-NMR (400 MHz, DMSO-d₆): 8.88 (t, J=5.6 Hz, 1H), 8.05 (s, 1H), 6.87 (s, 1H), 3.80 (t, J=4.4 Hz, 4H), 3.50 (t, J=4.8 Hz, 4H), 3.38 (q, J=6 Hz, 2H), 2.43 (t, J=6.8 Hz, 2H), 2.18 (s, 6H).

### Step 2: N-(2-(dimethylamino)ethyl)-7-morpholino-5-(3-(m-tolyl)-1H-pyrazol-1-yl)thieno[3,2-b]pyridine-2-carboxamide

Following the procedure according to Example 268, 5-chloro-N-(2-(dimethylamino)ethyl)-7-morpholinothieno[3,2-b]pyridine-2-carboxamide (55 mg) is reacted with 3-(m-tolyl)-1H-pyrazole (23 mg) to provide a brown gummy solid, which is purified by preparative HPLC to afford the title compound (30 mg; 41%) as an off white solid. Mass [m/z]=491.3 [M+H]⁺; ¹H-NMR (400 MHz, DMSO-d₆): 8.86 (t, J=5.6 Hz, 1H), 8.70 (d, J=2.4 Hz, 1H), 8.15 (s, 1H), 7.80 (d, J=9.2 Hz, 2H), 7.52 (s, 1H), 7.38 (t, J=7.6 Hz, 1H), 7.22 (d, J=7.2 Hz, 1H), 7.08 (d, J=2.4 Hz, 1H), 3.87 (t, J=4.4 Hz, 4H), 3.56 (t, J=4.8 Hz, 4H), 3.41 (q, J=6 Hz, 2H), 2.45 (t, J=6.8 Hz, 2H), 2.40 (s, 3H), 2.22 (s, 6H).

The compounds of Examples 87-131, 133-142, 144-164, 219-262, and 266 may be made according to similar procedures as the foregoing.

### Example 270: In Vitro Inhibitory Activity

Compounds are tested for PIKFYVE inhibitory activity using the KINOMEscan™ Kinase Assay (Eurofins).

Kinase-tagged T7 phage strains are prepared in an E. coli host derived from the BL21 strain. E. coli are grown to log-phase and infected with T7 phage, and incubated with shaking at 32° C. until lysis. The lysates are centrifuged and filtered to remove cell debris.

Alternatively, some kinases are produced in HEK-293 cells and subsequently tagged with DNA for qPCR detection.

Streptavidin-coated magnetic beads are treated with biotinylated small molecule ligands for 30 minutes at room temperature to generate affinity resins for kinase assays. The liganded beads are blocked with excess biotin and washed with blocking buffer (Sea Block, 1% BSA, 0.05% Tween-20, 1 mM DTT) to remove unbound ligand and to reduce non-specific binding. Binding reactions are assembled by combining kinases, liganded affinity beads, and test compounds in 1×binding buffer (20% Sea Block, 0.17×PBS, 0.05% Tween-20, 6 mM DTT). Test compounds are prepared as 111× stocks in DMSO. Kd values are determined using an 11-point, 3-fold compound dilution series with three DMSO control points. Compounds are distributed by non-contact acoustic transfer and are then directly diluted into the assay for a final concentration of DMSO of 0.9%, with a final volume in each well of 0.02 mL. Assay plates are incubated at room temperature with shaking for one hour, and the affinity beads are then washed with wash buffer (1% PBS, 0.05% Tween-20). The beads are then resuspended in elution buffer (1×PBS, 0.05% Tween-20, 0.05 μM non-biotinylated affinity ligand) and incubated with shaking for 30 minutes. The concentration of kinase in the eluates is measured by qPCR. Binding constants (Kd) are calculated with a standard dose-response curve using the Hill equation, fitted using non-linear least squares fit with the Levenberg-Marquedt algorithm.

The KINOMEscan™ experiments provide the following results:

| PIKfyve | Symbol |
|---|---|
| IC₅₀ | |
| <1 nM | * |
| 1-10 nM | ** |
| 10-100 nM | *** |
| >100 nM | **** |

| PIKfyve % inhibition at | | | |
|---|---|---|---|
| 1 nM | 5 nM | 10 nM | Symbol |
| 52-100% | | | + |
| 25-32% | 33-75% | 50-75% | ++ |
| | 10-32% | 10-50% | +++ |

| Compound | PIKfyve Activity |
|---|---|
| Ex. 1 | ** |
| Ex. 2 | * |
| Ex. 3 | ** |
| Ex. 4 | * |
| Ex. 5 | 100% @ 50 nM |
| Ex. 6 | 100% @ 50 nM |
| Ex. 7 | 100% @ 50 nM |
| Ex. 9 | ** |
| Ex. 10 | * |
| Ex. 11 | ** |
| Ex. 12 | ** |
| Ex. 13 | ** |
| Ex. 14 | ** |
| Ex. 15 | * |
| Ex. 16 | * |
| Ex. 17 | * |
| Ex. 18 | * |
| Ex. 19 | * |
| Ex. 20 | * |
| Ex. 21 | * |
| Ex. 22 | * |
| Ex. 23 | * |
| Ex. 24 | * |
| Ex. 25 | * |
| Ex. 26 | ** |
| Ex. 27 | * |
| Ex. 28 | * |
| Ex. 29 | * |
| Ex. 30 | ** |
| Ex. 31 | ** |
| Ex. 32 | * |
| Ex. 33 | **** |
| Ex. 34 | ** |
| Ex. 35 | 13% @ 1 nM |
| Ex. 36 | * |
| Ex. 37 | * |
| Ex. 38 | * |
| Ex. 39 | * |
| Ex. 40 | ** |
| Ex. 41 | * |
| Ex. 42 | 98% @ 1000 nM |
| Ex. 43 | * |
| Ex. 44 | * |
| Ex. 45 | * |
| Ex. 46 | * |
| Ex. 47 | * |
| Ex. 48 | ** |
| Ex. 49 | * |
| Ex. 50 | ** |
| Ex. 51 | * |
| Ex. 52 | * |
| Ex. 53 | 17% @ 1 nM |
| Ex. 54 | * |
| Ex. 55 | * |
| Ex. 56 | 94% @ 3 nM |
| Ex. 57 | ** |
| Ex. 58 | * |
| Ex. 59 | ** |
| Ex. 60 | ** |
| Ex. 61 | * |
| Ex. 62 | ** |
| Ex. 63 | ** |
| Ex. 64 | ** |
| Ex. 65 | ** |
| Ex. 66 | * |
| Ex. 67 | * |
| Ex. 68 | * |
| Ex. 69 | * |
| Ex. 70 | * |
| Ex. 71 | *** |
| Ex. 72 | * |
| Ex. 73 | *** |
| Ex. 74 | * |
| Ex. 75 | ** |
| Ex. 76 | * |
| Ex. 77 | +++ |
| Ex. 78 | + |
| Ex. 79 | + |
| Ex. 82 | + |
| Ex. 83 | + |
| Ex. 84 | + |
| Ex. 85 | + |
| Ex. 86 | + |
| Ex. 129 | 92% @ 30 nM |
| Ex. 132 | + |
| Ex. 165 | 22% @ 3 nM |
| Ex. 166 | ++ |
| Ex. 167 | 0% @ 3 nM |
| Ex. 168 | 23% @ 3 nM |
| Ex. 169 | ++ |
| Ex. 170 | ++ |
| Ex. 172 | ++ |
| Ex. 173 | 8% @ 5 nM |
| Ex. 174 | +++ |
| Ex. 175 | ++ |
| Ex. 177 | + |
| Ex. 178 | ++ |
| Ex. 179 | + |
| Ex. 181 | ++ |
| Ex. 182 | ++ |
| Ex. 183 | + |
| Ex. 184 | 5% @ 5 nM |
| Ex. 185 | ++ |
| Ex. 186 | + |
| Ex. 187 | + |
| Ex. 188 | ++ |
| Ex. 189 | ++ |
| Ex. 190 | 5% @ 5 nM |
| Ex. 191 | ++ |
| Ex. 192 | ++ |
| Ex. 193 | 22% @ 5 nM |
| Ex. 194 | ++ |
| Ex. 195 | ++ |
| Ex. 196 | ++ |
| Ex. 197 | ++ |
| Ex. 198 | ++ |
| Ex. 199 | ++ |
| Ex. 200 | + |
| Ex. 201 | + |
| Ex. 202 | + |
| Ex. 203 | ++ |
| Ex. 204 | ++ |
| Ex. 205 | ++ |
| Ex. 206 | ++ |
| Ex. 208 | ++ |
| Ex. 209 | + |
| Ex. 210 | ++ |
| Ex. 211 | ++ |
| Ex. 212 | 0% @ 5 nM |
| Ex. 213 | +++ |
| Ex. 214 | ++ |
| Ex. 215 | +++ |
| Ex. 216 | +++ |
| Ex. 217 | TBD |
| Ex. 218 | TBD |
| Ex. 263 | TBD |
| Ex. 264 | TBD |
| Ex. 265 | TBD |
| Ex. 266 | TBD |
| Ex. 268 | TBD |
| Ex. 269 | TBD |

These results demonstrate that the Compounds of the Invention are highly potent inhibitors of PIKFYVE.

### Example 271: Anti-Viral Activity

Selected compounds are evaluated for their inhibitory activity against SARS-CoV-2, influenza A, Ebola, and Marburg using the immunofluorescence assay (IFA).

SARS-CoV-2 IFA: Test compounds are solubilized in DMSO to prepare 20 mg/mL stock solutions. Compounds are then serially diluted using eight half-log dilutions in test media so that the starting (high) test concentration is 100 μg/mL. Each dilution is added to 5 wells of a 96-well plate with 80-100% confluent MDCK cells. Three wells of each dilution are infected with virus (strain USA-WA1/2020 or Delta strain B.1.617), and two wells remain uninfected as toxicity controls. As virus controls, six wells are infected but untreated, and as cell controls, six wells are uninfected and untreated. Each virus is prepared to achieve an MOI (multiplicity of infection) of 0.001. A positive control compound is tested in parallel. Plates are incubated at 37° C. under 5% CO₂ atmosphere. On day 3 post-infection (once untreated virus control cells reached maximum cytopathic effect, CPE), plates are stained with neutral red dye for 2 hours. Supernatant dye is removed and the wells are rinsed with PBS. The incorporated dye is extracted using 50:50 Sorensen citrate buffer/ethanol for at least 30 minutes, and the optical density is read at 450 nM on a spectrophotometer. Optical densities are converted to percent of cell controls and normalized to virus control, then the concentration of test compound required to inhibit CPE by 50% (EC₅₀) is calculated by regression analysis. The concentration of compound that causes 50% cell death in the absence of virus is also calculated (CC₅₀). The selectivity index (SI) is calculated as CC₅₀ divided by EC₅₀.

Influenza A: The procedure above is followed using influenza A strain California/07/09 (H1N1)pdm09. Alternatively, the procedure above is followed, with the following minor modifications: Test compound is solubilized to form a 2 mg/mL DMSO stock solution; serial dilutions are prepared using a starting (high) test concentration of 10 μg/mL; maximum CPE is reached on day 5 post-infection.

Ebola and Marburg: Neutralization activity (IC₅₀) is evaluated against the Ebola virus (Mayinga strain) and Marburg virus (Angola strain) glycoproteins using an rVSV-pseudotype-based neutralization assay in Vero cells, with cytotoxicity run in parallel. The assays are performed in serum-free medium (GIBCO VP-SFM #11681-020). The neutralization assays are luciferase-based microneutralization assays. Vero cells are seeded in black 96-well plates on day −1 at 50,000 cells per well. Eight semi-log serial dilutions are prepared and incubated for 1-hour with approximately 30,000 RLU of rVSV-EBOV-GP or rVSV-MARV-GP. Virus-only and cells-only are added as controls for calculation, as well as an internal assay control for assay validation. The test article formulation is 330 μM in DMSO. The test article/virus mixture is then added in triplicate to the Vero cells and the plates are incubated for 24-hours at 37° C. Firefly Luciferase activity is detected using the Bright-Glo™ Assay System kit (Promega). Fifty percent inhibition concentration (IC50) is calculated using XLfit dose response model. For the cytotoxicity assay, Vero cells are seeded in black 96-well plates on day −1 at 50,000 cells per well. The same dilutions of the TAs are prepared in triplicate and added to Vero cells for one-day incubation at 37° C.; cells-only and medium-only wells are also tested in parallel. On Day 1, cells are lysed for evaluation of the ATP content using Promega's CellTiter-Glo® kit. Luciferase luminescence in relative light unit (RLU) is read and 50% cytotoxicity concentration (CC50) is calculated using the XLFit dose response model.

The results are summarized in the table below, with comparison to the reference compound remdesivir for the SARS-CoV2 assay (remdesivir EC₅₀ 5.6 μM and CC₅₀>100 μM), and ribavirin for the influenza A assay (ribavirin EC₅₀ 3.8 μM and CC₅₀>1000 μM):

| Test Material | Virus | EC₅₀ | CC₅₀ | Selectivity Index |
|---|---|---|---|---|
| Ex. 2 | SARS-COV2 | <1 μM | >150 μM | >150 |
| Ex. 2 | SARS-COV2 | <3 μM | >3000 μM | >1000 |
| | (Delta) | | | |
| Ex. 2 | Influenza A | <1 μM | >150 μM | >150 |
| Ex. 2 | Ebola | <0.5 μM | >3000 μM | >6000 |
| Ex. 2 | Marburg | <0.2 μM | >3000 μM | >15000 |

### Example 272: Anti-Proliferative Activity

Selected compounds are evaluated for their anti-proliferative activity against human cutaneous T-cell lymphoma and multiple myeloma cell lines.

HuT-78 (T cell lymphoma) cells or JJN3 (multiple myeloma) cells are cultured in RPMI-1640 with 10% FBS and 1% penicillin/streptomycin. 45 μL suspensions of cells are transferred to the wells of 384-well plate for a density of 1,000 cells per well. The plates are incubated overnight at 37° C. under 5% CO₂ atmosphere. Test compounds are dissolved in 100% DMSO at a 2 mM concentration, then diluted 20× in assay medium for to provide a 5% DMSO concentration and 100 μM compound concentration. 5 μL of this compound solution is added to the 45 μL cell suspension in the assay plate for a top plate concentration of 10 μM and 0.5% DMSO concentration. For positive controls, 10 μM APY0201 is added instead of the compound solution. After spinning at 1000 rpm for 1 minutes, the cell plate is placed in the incubator overnight at 37° C. under 5% CO₂ atmosphere. After incubation for six days, the cell plate is equilibrated to room temperature for 20 minutes. 25 μL of CellTiter-Glo (CTG) reagent (Promega Cat No. G7572) is added to each well, and the plate is shaken for 15 minutes at 300 rpm. Luminescence is read on an Envision plate reader.

The results are shown in the table below:

| Test Material | Cell Line | EC50 |
|---|---|---|
| Ex. 2 | HuT-78 | <250 nM |
| Ex. 2 | JJN3 | <50 nM |

1. A compound of Formula I: 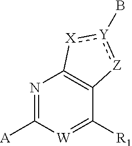
in free or pharmaceutically acceptable salt form, wherein
(i) X is selected from —CH—, —CR₃—, —S—, —O—, —N—, —NH—, and —NR₃—;
(ii) Y is selected from —C— and —N—;
(iii) Z is selected from —CH—, —CR₃—, —S—, —O—, —N—, —NH—, and —NR₃—;
(iv) W is —CH—, —CR₄—, or —N—;
(v) A is an optionally substituted heteroaryl (e.g., 5-membered heteroaryl) or heterocycloalkyl (e.g., 3- to 6-membered heterocycloalkyl);
(vi) B is halo, an optionally substituted aryl, optionally substituted heteroaryl, optionally substituted C₃₋₆cycloalkyl, optionally substituted 3- to 6-membered heterocycloalkyl, optionally substituted 3- to 6-membered heterocycloalkenyl, optionally substituted C₁₋₆alkyl, optionally substituted C₂₋₆alkenyl (e.g., vinyl), —N(Rₐ)—R₂, —O—R₂, —(CO)—R₂, —(CO)—O—R₂, —(CO)—N(Rₐ)—R₂, —O—(CO)—R₂, —N(Rₐ)—(CO)—R₂, —(CO)—N(Rₐ)—(CO)—R₂, N(Rₐ)—(CO)—N(Rₐ)—R₂, optionally substituted —(C₁₋₆alkyl)-(3- to 6-membered heterocycloalkyl), optionally substituted —(C₁₋₆alkyl)-(C₃₋₆cycloalkyl), optionally substituted —(C₂₋₆alkenyl)-(3- to 6-membered heterocycloalkyl), optionally substituted —(C₂₋₆alkenyl)-(C₃₋₆cycloalkyl), optionally substituted —(C₂₋₆alkynyl)-(3- to 6-membered heterocycloalkyl), optionally substituted —(C₂₋₆alkynyl)-(C₃₋₆cycloalkyl), optionally substituted —(C₁₋₆alkyl)-N(Rₐ)—R₂, optionally substituted —(C₁₋₆alkyl)-O—R₂, optionally substituted —(C₂₋₆alkenyl)-N(Rₐ)—R₂, optionally substituted —(C₂₋₆alkenyl)-O—R₂, optionally substituted —(C₂₋₆ alkenyl)-N(Rₐ)(CO)—R₂, optionally substituted —(C₂₋₆alkenyl)-O(CO)—R₂, optionally substituted —(C₂₋₆alkynyl)-N(Rₐ)—R₂, optionally substituted —(C₂₋₆alkynyl)-O—R₂, optionally substituted —(C₂₋₆alkynyl)-N(Rₐ)(CO)—R₂, optionally substituted —(C₂₋₆alkynyl)-O(CO)—R₂, optionally substituted —(C₁₋₆alkyl)-(CO)—N(Rₐ)—R₂, optionally substituted —O—(C₁₋₆alkyl)-R₂, optionally substituted —N(Rₐ)—(C₁₋₆alkyl)-R₂, optionally substituted —CH₂—(3- to 6-membered heterocycloalkyl), optionally substituted —CH₂—(C₃₋₆cycloalkyl), optionally substituted —CH₂—N(Rₐ)—R₂, optionally substituted —CH₂—O—R₂, or optionally substituted —CH₂—(CO)—N(Rₐ)—R₂, optionally substituted —(CO)-(3- to 6-membered heterocycloalkyl), optionally substituted —(CO)—(C₃₋₆cycloalkyl), optionally substituted (6- to 12-membered bicyclic heterocycloalkyl), optionally substituted —CH₂—(6- to 12-membered bicyclic heterocycloalkyl), or optionally substituted —(CO)-(6- to 12-membered bicyclic heterocycloalkyl);
(vii) R₁ is an optionally substituted C₁₋₆alkyl, optionally substituted C₃₋₆cycloalkyl, optionally substituted C₁₋₆alkoxy, optionally substituted 3- to 7-membered heterocycloalkyl, optionally substituted 6- to 12-membered bicyclic heterocycloalkyl), —C(O)—R₂, —C(O)O—R₂, —OC(O)—R₂, —C(O)N(Rₐ)—R₂, —N(Rₐ)C(O)—R₂, —N(Rₐ)—R₂, or —O—R₂;
(viii) Rₐ is H, optionally substituted C₁₋₆alkyl, or optionally substituted C₃₋₆cycloalkyl; and
(ix) R₂ is optionally substituted C₁₋₆alkyl, optionally substituted C₃₋₆cycloalkyl, optionally substituted C₁₋₆alkoxy, optionally substituted aryl, optionally substituted heteroaryl, or optionally substituted 3- to 7-membered heterocycloalkyl;
(x) R₃ is H, optionally substituted C₁₋₆alkyl (e.g., methyl), optionally substituted C₃₋₆cycloalkyl, optionally substituted haloC₁₋₆alkyl (e.g., CF₃), or optionally substituted 3- to 6-membered heterocycloalkyl; and
(xi) R₄ is halogen (e.g., fluoro), —OH, —NH₂, C₁₋₆alkyl (e.g., methyl), C₃₋₆cycloalkyl (e.g., isopropyl), haloC₁₋₆alkyl (e.g., CF₃), C₁₋₆alkoxy (e.g., methoxy), —NH(C₁₋₆alkyl) (e.g., methylamino), or —N(C₁₋₆alkyl)(C₁₋₆alkyl) (e.g., dimethylamino);
provided that
(a) when A is an optionally substituted pyrazole, said pyrazole is substituted by at least one optionally substituted aryl (e.g., phenyl) ring;
(b) when A is optionally substituted pyrazol-1-yl, X is not O when Y is —CH— and Z is —CH—;
(c) when A is optionally substituted pyrazol-1-yl, and X is —CH—, Y is —C—, and Z is —O—, B is not pyridyl, pyrimidinyl, any 5-membered heteroaromatic ring (e.g., thiazolyl, oxazolyl, pyrazolyl, isoxazolyl, isothiazolyl or imidazolyl), or —(CO)—N(Rₐ)—R₂;
(d) when A is 3-(m-tolyl)-pyrazol-1-yl, and X is —CH—, Y is —C—, and Z is —O—, and B is —(CO)-(6-12 membered heterocycloalkyl), said heterocycloalkyl in group B is not unsubstituted morpholine, unsubstituted piperidine, unsubstituted pyrrolidine, unsubstituted piperazine, or 4-methylpiperazine;
(e) when B is W is —N—, X is —N—, —NH—, or —NCH₃—, or Z is —N—, —NH—, or —NCH₃—, and Y is —C—, then B is not pyridyl, unsubstituted pyrrolidinyl, unsubstituted piperidinyl, 1-methyl-4-piperidinyl, 2-methyl-4-piperidinyl, tetrahydropyranyl, dihydropyranyl, 1-tert-butoxycarbonyl-4-azetidinyl, 1-methyl-4-azetidinyl, 4-azetidinyl, 1,2-dihydroxy-1-ethyl;
(f) when W is —CH—, X is —CH— and Y is —N—, B is not 2-(1-methyl-pyrazol-3-yl)ethyl;
(g) when B is pyrid-4-yl, R₁ is morpholin-4-yl, X is —CH—, Y is —C—, and Z is —S—, A is not 3-(3-methylphenyl)-5-hydroxy-pyrazol-1-yl, 3-(3-methoxyphenyl)-5-hydroxy-pyrazol-1-yl, or 3-(3-isopropoxyphenyl)-5-hydroxy-pyrazol-1-yl; and
(h) when B is phenyl, R₁ is N-cyclohexylamino, X is —O—, Y is —C—, and Z is —CH—, A is not 3,5-diphenyl-pyrazol-1-yl, 3-methyl-5-phenyl-pyrazol-1-yl, 3-trifluoromethyl-5-phenyl-pyrazol-1-yl, or 3,5-dimethyl-4-phenyl-pyrazol-1-yl;
(i) when W is —N—, X is —CH— or —C(Me)-, Y is —C—, Z is —S— or —O—, and R₁ is 4-morpholinyl, then A is not 1H-indazol-4-yl, 1H-indol-4-yl, 1H-pyrrolo[2,3-b]pyridin-5-yl, or 2-methyl-3H-imidazo[4,5]pyridin-6-yl;
(j) when W is —N—, X is —S— or —O—, Y is —C—, Z is —CH— or —C(Me)-, and R₁ is 4-morpholinyl, then A is not 1H-indazol-4-yl, 1H-indol-4-yl, 1H-pyrrolo[2,3-b]pyridin-5-yl, or 2-methyl-3H-imidazo[4,5]pyridin-6-yl;
(k) when W is —N—, X is —CH— or —C(Me)-, Y is —C—, Z is —S— or —O—, B is H or optionally substituted C₁₋₆alkyl, and R₁ is 4-morpholinyl, then A is not 2-oxoindolin-4-yl, 1-acetylindolin-4-yl, 1H-indazol-6-yl, 1H-indol-5-yl, 1H-indol-6-yl, or quinolin-3-yl;
(l) when W is —N—, X is —S— or —O—, Y is —C—, Z is —CH— or —C(Me)-, B is H or optionally substituted C₁₋₆alkyl, and R₁ is 4-morpholinyl, then A is not 2-oxoindolin-4-yl, 1-acetylindolin-4-yl, 1H-indazol-6-yl, 1H-indol-5-yl, 1H-indol-6-yl, or quinolin-3-yl;
(m) when W is —N—, X is —S—, Z is —N—, Y is —C—, and R₁ is 4-morpholinyl, then A is not optionally substituted pyrid-3-yl, optionally substituted pyrimidin-5-yl, unsubstituted quinolin-3-yl, unsubstituted 1H-indazol-4-yl, unsubstituted 1H-indol-4-yl, or unsubstituted 1H-pyrrolo[2,3-b]pyridin-5-yl;
(n) when W is —N—, X is —N—, Z is —S—, Y is —C—, and R₁ is 4-morpholinyl, then A is not optionally substituted pyrid-3-yl, optionally substituted pyrimidin-5-yl, unsubstituted quinolin-3-yl, unsubstituted 1H-indazol-4-yl, unsubstituted 1H-indol-4-yl, or unsubstituted 1H-pyrrolo[2,3-b]pyridin-5-yl,
(o) when W is —N—, X is —S—, Z is —N—, Y is —C—, and A is unsubstituted indolyl, pyrazolyl, imidazolyl, or triazolyl, then R₁ is not 3-methylmorpholin-4-yl
(p) when W is —N—, X is —NR₃—, R₃ is selected from methyl, ethyl, isopropyl, and cyclopentyl, Z is —N—, Y is —C—, and R₁ is 4-morpholinyl, then A is not benzo[d][1,3]dioxol-5-yl, pyrimidin-5-yl, 2-aminopyrimidin-5-yl, pyridin-3-yl, 6-aminopyridin-3-yl, 6-methoxypyridin-3-yl, quinolin-3-yl, or pyridin-4-yl;
(q) when W is —N—, X is —NR₃—, R₃ is 1-benzylpiperidin-4-yl, Z is —CH—, Y is —N—, and R₁ is 4-morpholinyl, then A is not pyridin-3-yl, pyridin-4-yl, quinolin-3-yl, quinolin-6-yl, 1H-indol-2-yl, 1H-indol-5-yl, 1H-indol-6-yl, 1H-indol-7-yl, 1-methyl-indol-5-yl, or 2-oxo-2,3-dihydro-1H-benzo[d]imidazol-5-yl;
(r) when W is —N—, X is —CH— or —C(Me)-, Y is —C—, Z is —S— or —O—, and R₁ is 4-morpholinyl, then A is not 2-aminopyrimidin-5-yl;
(s) when W is —N—, Y is —C—, and R₁ is 4-morpholinyl, A is not 2-methyl-1H-benzo[d]imidazole-1-yl or 2-ethyl-1H-benzo[d]imidazol-1-yl;
(t) when W is —N—, X is —NR₃—, R₃ is methyl, Z is —N—, Y is —C—, and B is optionally substituted —CH₂-piperidine, optionally substituted —CH₂-piperazine, —CH₂-azetidine, or 2,5-diazabicyclo[2.2.1]heptan-2-yl, then A is not 2-methyl-1H-benzo[d]imidazol-1-yl or 2-ethyl-1H-benzo[d]imidazol-1-yl;
(u) when W is —N—, X is —CH—, Y is —C—, Z is —S—, and R₁ is 4-morpholinyl, then A is not 5-fluoro-1H-indol-4-yl, 6-fluoro-1H-indol-4-yl, 6-cyano-1H-indol-4-yl, 5,7-difluoro-1H-indol-4-yl, 2-methyl-1H-indol-4-yl, 1-methyl-1H-indol-4-yl, 1H-pyrrolo[2,3-c]pyridin-4-yl, 1H-pyrrolo[2,3-b]pyridin-4-yl, 2-oxo-1H-indolin-4-yl, imidazo[1,2-a]pyridin-5-yl, 2-methyl-quinolin-5-yl, 2-methylbenzo[b]thiophen-3-yl, 1H-indazol-3-yl, 1H-indol-3-yl, 1H-benzo[d]imidazole-1-yl, 1H-indazol-1-yl, 1H-indol-1-yl, 2-methyl-benzo[d]imidazole-1-yl, or 3-methylbenzo[d]imidazole-1-yl;
(v) when W is —N—, X is —CH— or —C(Me)-, Y is —C—, Z is —S— or —O—, and R₁ is 4-morpholinyl, then A is not optionally substituted 1H-benzo[d]imidazol-1-yl, optionally substituted quinolin-5-yl, optionally substituted 1H-pyrrolo[2,3-c]pyridin-4-yl, optionally substituted 1H-pyrrolo[2,3-c]pyridin-3-yl, optionally substituted 1H-pyrrolo[3,2-c]pyridin-4-yl, optionally substituted isoquinolin-8-yl, optionally substituted isoquinolin-4-yl, optionally substituted isoquinolin-5-yl, optionally substituted 1H-indazol-1-yl, optionally substituted 1H-indazol-3-yl, optionally substituted benzofuran-3-yl, optionally substituted 1H-pyrrolo[2,3-b]pyridin-3-yl, optionally substituted 1H-pyrrolo[2,3-b]pyridin-5-yl, optionally substituted 1H-pyrrolo[2,3-b]pyridin-4-yl, optionally substituted benzo[b]thiophen-3-yl, optionally substituted 1H-pyrolo[3,2-c]-pyridin-1-yl, 2-oxo-1H-benzo[d]imidazol-1-yl, optionally substituted 1H-benzo[d]imidazol-1-yl, optionally substituted 2H-indazol-3-yl, 2-oxo-1-indolin-1-yl, optionally substituted 2H-indazol-3-yl, optionally substituted imidazo[3,2-d]pyrimidin-6-yl, optionally substituted imidazo[1,5-a]pyridin-8-yl, optionally substituted imidazo[1,2-a]pyridin-5-yl, optionally substituted imidazo[4,5-c]pyridin-1-yl, optionally substituted imidazo[4,5-c]pyridin-3-yl, 1-oxo-1,2-dihydroisoquinolin-4-yl, 2-methyl-3-oxo-2,3-dihydro-1H-indazol-1-yl, [1,2,4]-triazolo[4,3-a]pyridin-5-yl, cinnolin-4-yl, or benzo[d]isothiazol-3-yl;
(w) when W is —N—, X is —S—, —NH— or —N(Me)-, Y is —C—, Z is —N—, and R₁ is 4-morpholinyl, then A is not optionally substituted 1H-benzo[d]imidazol-1-yl, optionally substituted quinolin-5-yl, optionally substituted 1H-pyrrolo[2,3-c]pyridin-4-yl, optionally substituted 1H-pyrrolo[2,3-c]pyridin-3-yl, optionally substituted 1H-pyrrolo[3,2-c]pyridin-4-yl, optionally substituted isoquinolin-8-yl, optionally substituted isoquinolin-4-yl, optionally substituted isoquinolin-5-yl, optionally substituted 1H-indazol-1-yl, optionally substituted 1H-indazol-3-yl, optionally substituted benzofuran-3-yl, optionally substituted 1H-pyrrolo[2,3-b]pyridin-3-yl, optionally substituted 1H-pyrrolo[2,3-b]pyridin-5-yl, optionally substituted 1H-pyrrolo[2,3-b]pyridin-4-yl, optionally substituted benzo[b]thiophen-3-yl, optionally substituted 1H-pyrolo[3,2-c]-pyridin-1-yl, 2-oxo-1H-benzo[d]imidazol-1-yl, optionally substituted 1H-benzo[d]imidazol-1-yl, optionally substituted 2H-indazol-3-yl, 2-oxo-1-indolin-1-yl, optionally substituted 2H-indazol-3-yl, optionally substituted imidazo[3,2-d]pyrimidin-6-yl, optionally substituted imidazo[1,5-a]pyridin-8-yl, optionally substituted imidazo[1,2-a]pyridin-5-yl, optionally substituted imidazo[4,5-c]pyridin-1-yl, optionally substituted imidazo[4,5-c]pyridin-3-yl, 1-oxo-1,2-dihydroisoquinolin-4-yl, 2-methyl-3-oxo-2,3-dihydro-1H-indazol-1-yl, [1,2,4]-triazolo[4,3-a]pyridin-5-yl, cinnolin-4-yl, or benzo[d]isothiazol-3-yl;
(x) when W is —N—, X is —NH—, Y is —C—, Z is —S—, and R₁ is 4-morpholinyl, then A is not optionally substituted 1H-benzo[d]imidazol-1-yl, optionally substituted quinolin-5-yl, optionally substituted 1H-pyrrolo[2,3-c]pyridin-4-yl, optionally substituted 1H-pyrrolo[2,3-c]pyridin-3-yl, optionally substituted 1H-pyrrolo[3,2-c]pyridin-4-yl, optionally substituted isoquinolin-8-yl, optionally substituted isoquinolin-4-yl, optionally substituted isoquinolin-5-yl, optionally substituted 1H-indazol-1-yl, optionally substituted 1H-indazol-3-yl, optionally substituted benzofuran-3-yl, optionally substituted 1H-pyrrolo[2,3-b]pyridin-3-yl, optionally substituted 1H-pyrrolo[2,3-b]pyridin-5-yl, optionally substituted 1H-pyrrolo[2,3-b]pyridin-4-yl, optionally substituted benzo[b]thiophen-3-yl, optionally substituted 1H-pyrolo[3,2-c]-pyridin-1-yl, 2-oxo-1H-benzo[d]imidazol-1-yl, optionally substituted 1H-benzo[d]imidazol-1-yl, optionally substituted 2H-indazol-3-yl, 2-oxo-1-indolin-1-yl, optionally substituted 2H-indazol-3-yl, optionally substituted imidazo[3,2-d]pyrimidin-6-yl, optionally substituted imidazo[1,5-a]pyridin-8-yl, optionally substituted imidazo[1,2-a]pyridin-5-yl, optionally substituted imidazo[4,5-c]pyridin-1-yl, optionally substituted imidazo[4,5-c]pyridin-3-yl, 1-oxo-1,2-dihydroisoquinolin-4-yl, 2-methyl-3-oxo-2,3-dihydro-1H-indazol-1-yl, [1,2,4]-triazolo[4,3-a]pyridin-5-yl, cinnolin-4-yl, or benzo[d]isothiazol-3-yl;
(y) when W is —N—, X is —N—, —NH— or —N(CH₂CH₂OH)—, Z is —N—, Y is —C—, and R₁ is 4-morpholinyl, then A is not 1H-indol-4-yl or 5-fluoro-1H-indol-4-yl;
(z) when W is —N— or —CH—, X is —NH—, —N(Me)-, —N(Et)-, or —N(CH₂-cyclopropyl)-, Y is —C—, Z is —N—, and R₁ is 4-morpholinyl, then A is not an unsubstituted pyrazol-4-yl or a pyrazol-4-yl substituted by one or more groups selected from methyl, ethyl, n-propyl, isopropyl, and trifluoromethyl;
(aa) when W is —N—, X is —NH—, Y is —C—, Z is —CH— or —N—, and R₁ is 4-morpholinyl, 3-methylmorpholin-4-yl, 3,3-dimethylmorpholin-4-yl, 2-oxa-5-azabicyclo[2.2.1]hept-5-yl, or 3-oxa-8-azabicyclo[3.2.1]oct-8-yl, then A is not 4-morpholinyl, 3-methylmorpholin-4-yl, piperidin-1-yl, 4-hydroxypiperidin-1-yl, 4-methoxypiperidin-1-yl, 4,4-difluoropiperidin-1-yl, or 8-oxa-3-azabicyclo[3.2.1]oct-3-yl;
(bb) when W is —CH—, X is —S—, Y is —C—, and X is —CH—, B is not pyrazol-5-yl when A is pyrrolidine-1-yl;
and wherein the compound of Formula I is not
4-(2-methyl-5-(3-(m-tolyl)-1H-pyrazol-1-yl)-1H-pyrrolo[3,2-b]pyridin-7-yl)morpholine,
2-(2-(2-aminopyrimidin-5-yl)-9-(2-hydroxyethyl)-6-morpholino-9H-purin-8-yl)propan-2-ol,
1-ethyl-3-(5-(6-(3-ethylmorpholino)-7-methyl-7H-purin-2-yl)pyrimidin-2-yl)urea, or
5-pyrrolidino-2-(4-methoxyphenyl)-2H-[1,2,3]triazolo[4,5-d]pyrimidin-7-one or a tautomer thereof.

2. The compound according to claim 1, wherein
(a) X is —S—, Z is —CH— or —CR₃—, and Y is —C—;
(b) X is —CH— or —CR₃—, Z is —S—, and Y is —C—;
(c) X is —O—, Z is —CH— or —CR₃—, and Y is —C—;
(d) X is —CH— or —CR₃—, Z is —O—, and Y is —C—;
(e) X is —NH—, Z is —CH— or —CR₃—, and Y is —C—;
(f) X is —CH— or —CR₃—, Z is —NH—, and Y is —C—;
(g) X is —NR₃—, Z is —CH— or —CR₃—, and Y is —C—;
(h) X is —CH— or —CR₃—, Z is —NR₃—, and Y is —C—;
(i) X and Z are —CH— or —CR₃—, and Y is —N—;
(j) X is —S—, Z is —N—, and Y is —C—;
(k) X is —N—, Z is —S—, and Y is —C—;
(l) X is —O—, Z is —N—, and Y is —C—;
(m) X is —N—, Z is —O—, and Y is —C—;
(n) X is —NH—, Z is —N—, and Y is —C—;
(o) X is —N—, Z is —NH—, and Y is —C—;
(p) X is —NR₃—, Z is —N—, and Y is —C—;
(q) X is —N—, Z is —NR₃—, and Y is —C—;
(r) X is —N—, Z is —CH— or —CR₃—, and Y is —N—;
(s) X is —CH— or —CR₃—, Z is —N—, and Y is —N—; or
(t) X and Z are —N—, and Y is —N—.

3. The compound according to claim 1, wherein the compound of Formula I has a core structure selected from any of the following:

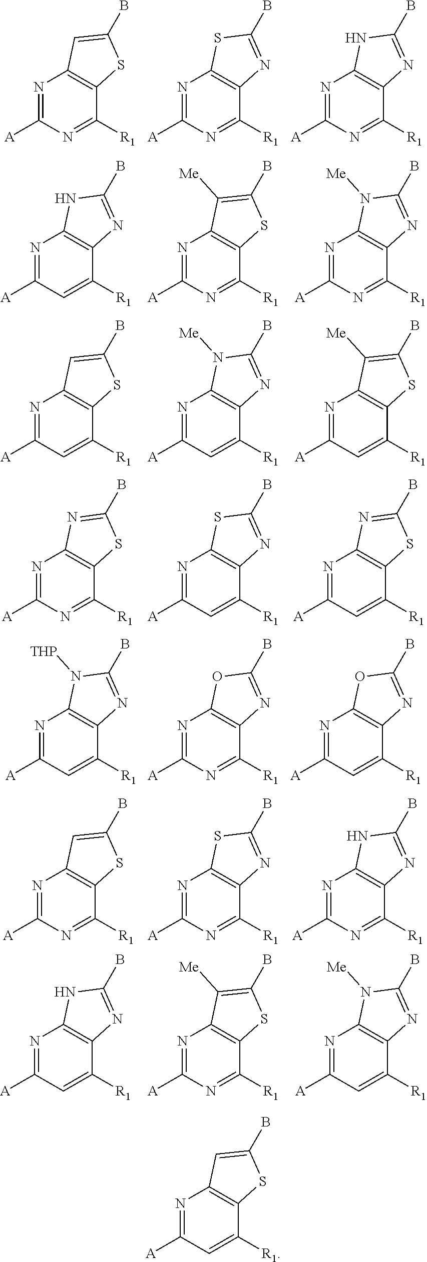

4. The compound according to claim 1, wherein A is selected from pyridine, pyrimidine, pyridazine, pyrazine, triazine, indole, benzimidazole, benzoxazole, benzothiazole, indazole, benzisoxazole, and benzisothiazole, thiophene, furan, pyrrole, oxazole, imidazole, thiazole, pyrazole, isoxazole, isothiazole, triazole (e.g., 1,2,3-triazole, or 1,2,4-triazole), oxadiazole (e.g., 1,2,3-oxadiazole, or 1,2,4-oxadiazole), thiadiazole (e.g., 1,2,3-thiadiazole, or 1,2,4-thiadiazole), and tetrazole (e.g., 1,2,3,4-tetrazole).

5. The compound according to claim 4, wherein said heteroaryl is selected from oxazole, imidazole, thiazole, pyrazole, isoxazole isothiazole, and triazole (e.g., 1,2,3-triazole).

6. The compound according to claim 5, wherein said heteroaryl is pyrazole (e.g., 3-substituted-pyrazol-1-yl, 1-substituted-pyrazol-3-yl, 2-substituted-pyrazol-4-yl, 4-substituted-pyrazol-1-yl, 1-substituted-pyrazol-5-yl, or 5-substituted-pyrazol-3-yl).

7. The compound according to claim 5, wherein said heteroaryl is thiazole (e.g., 2-substituted-thiazol-4-yl, 2-substituted-thiazol-5-yl, 4-substituted-thiazol-2-yl, or 5-substituted-thiazol-2-yl).

8. The compound according to claim 5, wherein said heteroaryl is oxazole (e.g., 2-substituted-oxazol-4-yl, 2-substituted-oxazol-5-yl, 4-substituted-oxazol-2-yl, or 5-substituted-oxazol-2-yl).

9. The compound according to claim 5, wherein said heteroaryl is imidazole (e.g., 2-substituted-imidazol-4-yl, 4-substituted-imidazol-2-yl, 2-substituted-imidazol-5-yl, 5-substituted-imidazol-2-yl, 4-substituted-imidazol-1-yl, or 1-substituted-imidazol-4-yl).

10. The compound according to claim 5, wherein said heteroaryl is triazole (e.g., 4-substituted-1,2,3-triazol-2-yl or 2-substituted-1,2,3-triazol-4-yl).

11. The compound according to claim 1, wherein substituent A is selected from:

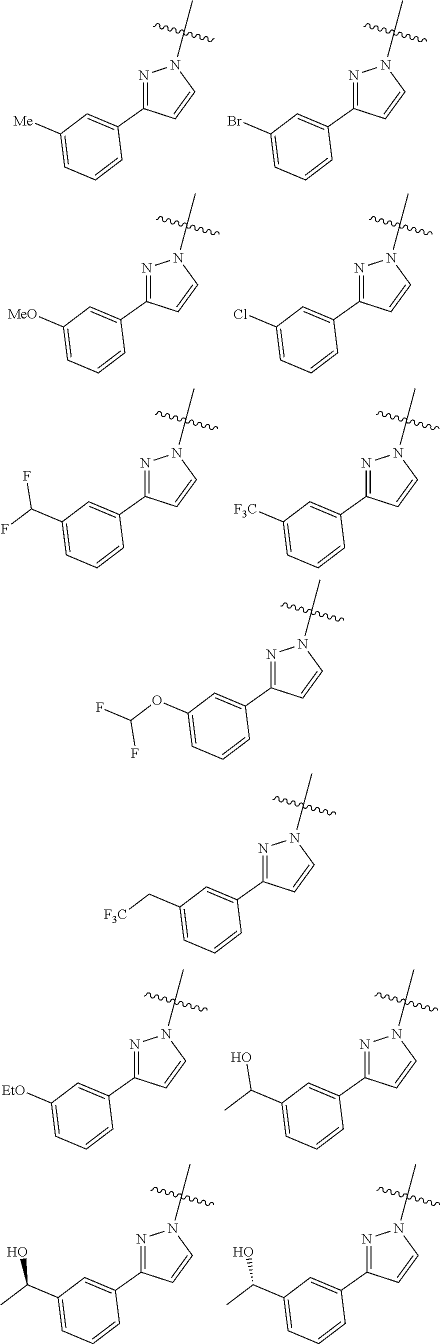

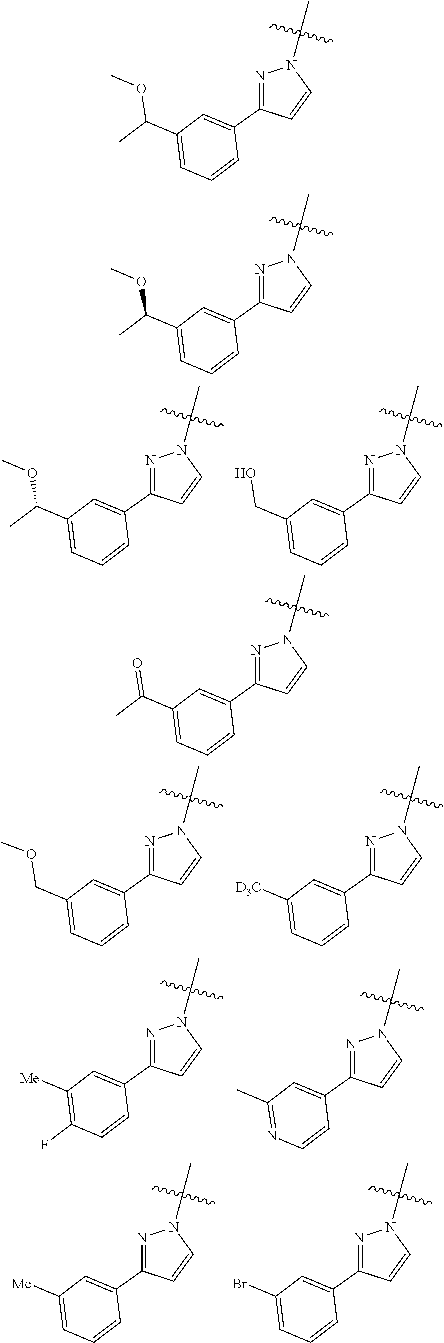

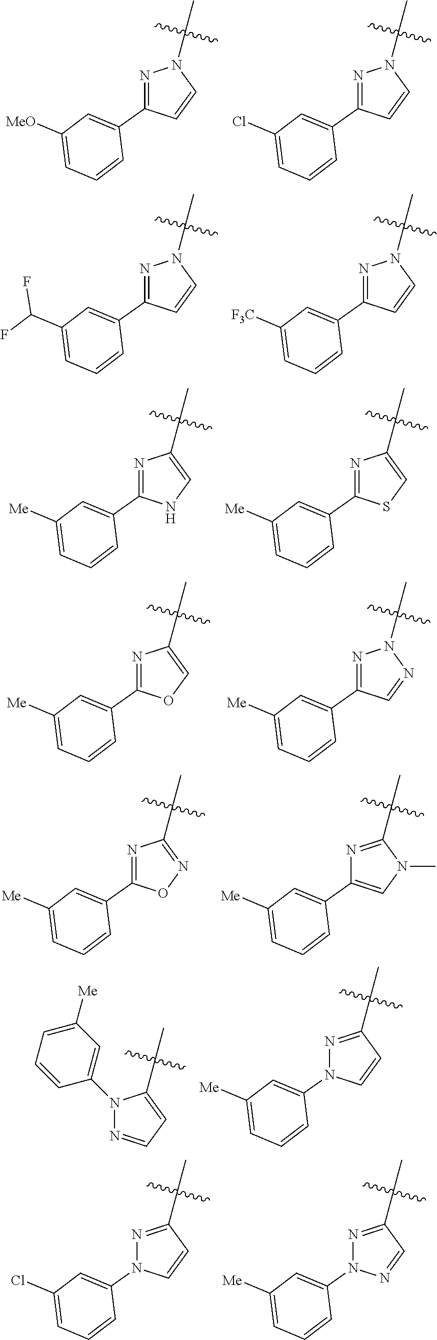

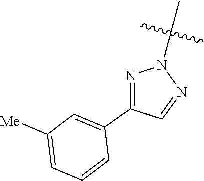 [SMILES: *n1ncc(-c2cccc(C)c2)n1]

12. The compound according to claim 1, wherein B is selected from halo (e.g., bromo), phenyl, pyridine, pyrimidine, pyridazine, pyrazine, triazine, tetrazine, thiophene, furan, pyrrole, oxazole, imidazole, thiazole, pyrazole, isoxazole, isothiazole, indole, indazole, benzimidazole, benzisoxazole, benzisothiazole, benzoxazole, and benzothiazole, each optionally an N-oxide thereof (e.g., pyridyl-N-oxide), and each optionally substituted.

13. The compound according to claim 1, wherein B is —CH₂—(3- to 6-membered heterocycloalkyl) or —(CO)-(3- to 6-membered heterocycloalkyl), and said heterocycloalkyl is selected from morpholine, piperidine, piperazine, tetrahydropyran, pyrrolidine, tetrahydrofuran, oxetane, azetidine, oxirane, and aziridine, each optionally substituted.

14. The compound according to claim 1, wherein group B is selected from the group consisting of:

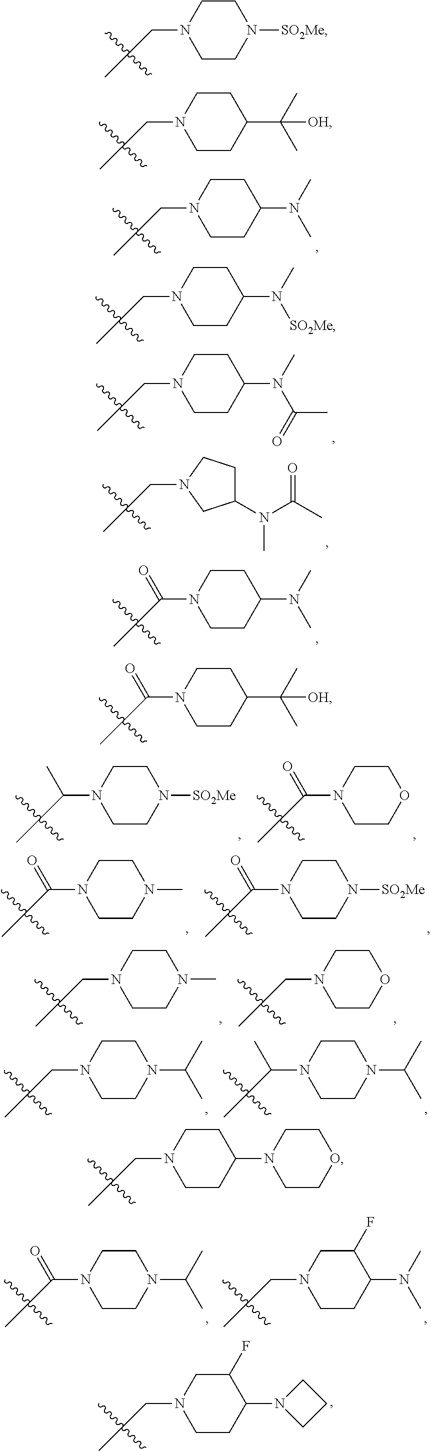

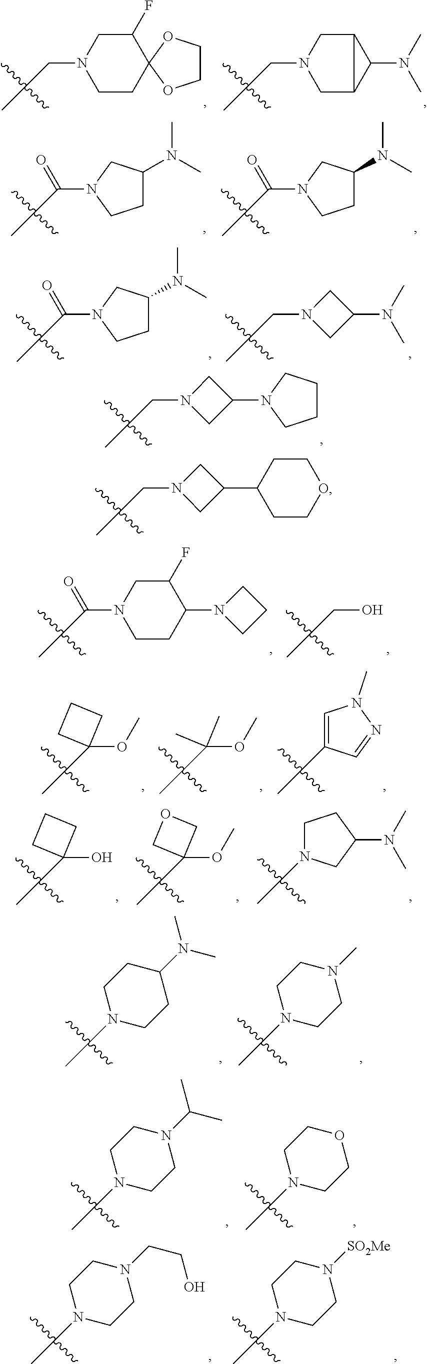

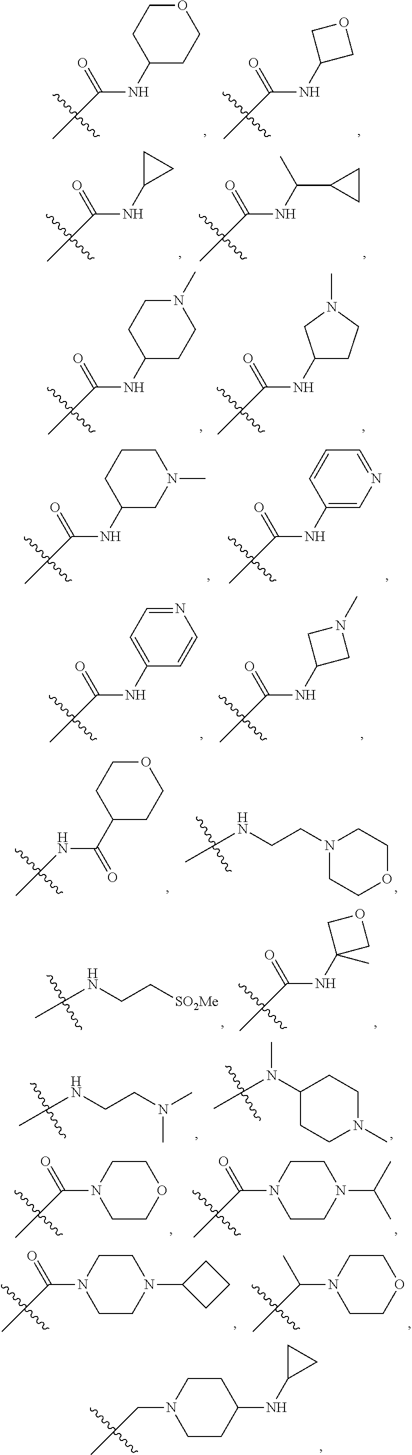

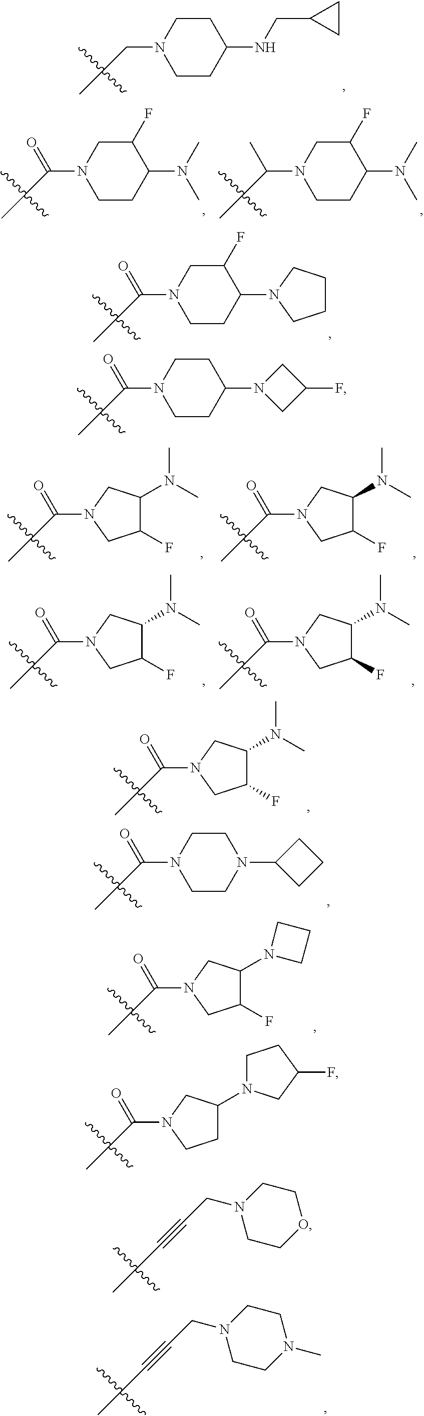

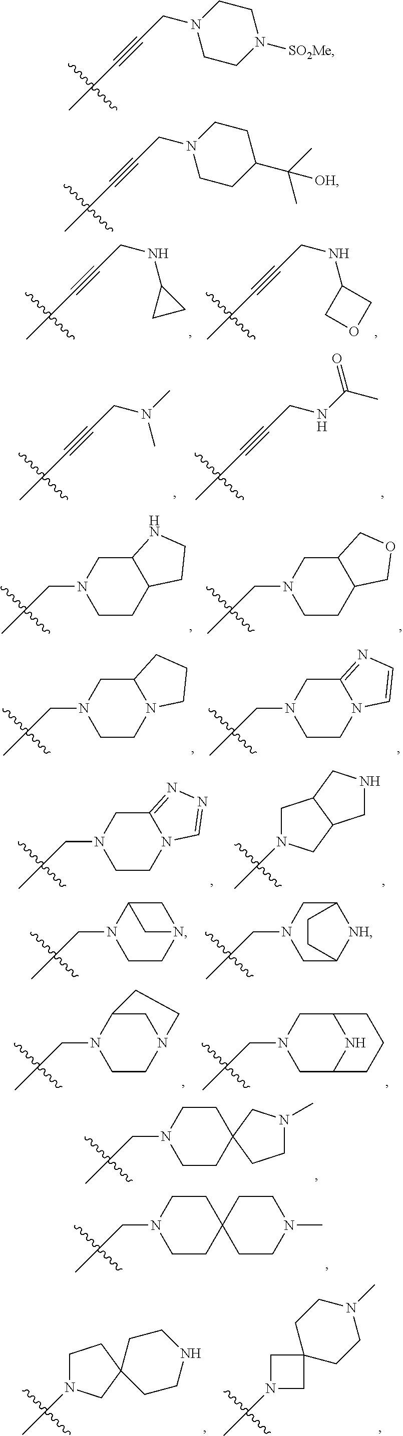

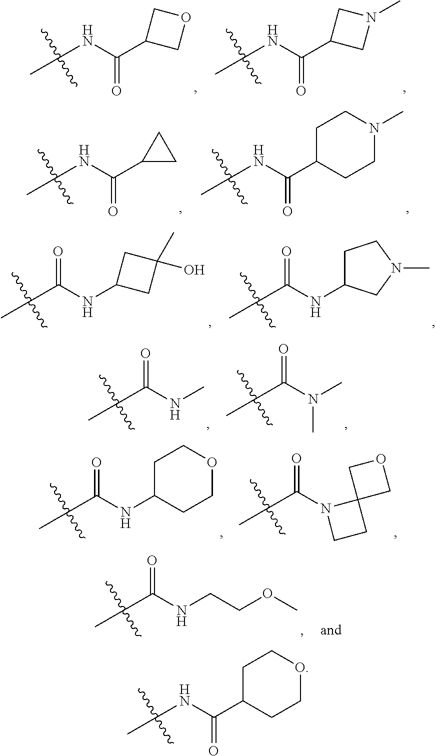

15. The compound according to claim 1, wherein R₁ is an optionally substituted 3- to 7-membered heterocycloalkyl.

16. The compound according to claim 15, wherein said heterocycloalkyl is selected from aziridine, azetidine, oxirane, oxetane, pyrrolidine (e.g., 3,3-difluoropyrrolidin-1-yl), pyrrolidinone (e.g., 1-pyrrolidin-3-one), tetrahydrofuran, tetrahydropyran (e.g., tetrahydropyran-4-yl), dihydropyran (e.g., 3,6-dihydropyran-4-yl), morpholine, piperidine, piperazine, and oxa-azaspiro[3.3]heptane (e.g., 2-oxa-6-azaspiro[3.3]heptan-6-yl), 1,4-oxazepan-4-yl, 3-oxa-6-azabicyclo[3.1.1]heptan-6-yl, and 2-oxa-5-azabicyclo[2.2.1]heptan-5-yl, wherein each said heterocycloalkyl is optionally substituted.

17. The compound according to claim 16, wherein said morpholine is unsubstituted (e.g., morpholin-4-yl).

18. The compound according to claim 1, wherein the compound is selected from the compounds listed in Table 1 and Table 2.

19. A pharmaceutical composition comprising the compound according to claim 1, in free or pharmaceutically acceptable salt form, in admixture with a pharmaceutically acceptable diluents or carrier.

20. A method for the treatment or prophylaxis of a disease or disorder characterized by dysregulation of phosphoinositide-mediated signal transduction pathways or which may be ameliorated by modulating (e.g., inhibiting) PIKFYVE-dependent signaling pathways or by modulating (e.g., inhibiting) endosome formation or trafficking, comprising administering to a patient in need thereof an effective amount of the compound according to claim 1, in free or pharmaceutically acceptable salt form.

21. A method for treating or preventing a viral infection by an enveloped virus, such as Ebola, influenza A, vesicular stomatitis virus, Lassa fever virus, lymphocytic choriomeningitis virus, and coronaviruses (including MERS-CoV, SARS-CoV and SARS-CoV-2), comprising administering to a patient in need thereof an effective amount of the compound according to claim 1, in free or pharmaceutically acceptable salt form.
